(19) United States
(12) Patent Application Publication
(10) Pub. No.: US 2004/0171062 A1
(43) Pub. Date: Sep. 2, 2004

(54) Methods for the design of molecular scaffolds and ligands

(75) Inventors: Klaus-Peter Hirth, San Francisco, CA (US); Michael Vance Milburn, Emeryville, CA (US)

Correspondence Address: FOLEY & LARDNER, P.O. BOX 80278, SAN DIEGO, CA, 92138-0278, US

(73) Assignee: Plexxikon, Inc.

(21) Appl. No.: 10/377,268

(22) Filed: Feb. 28, 2003

Related U.S. Application Data
(60) Provisional application No. 60/437,929, filed on Jan. 2, 2003.
(60) Provisional application No. 60/360,651, filed on Feb. 28, 2002.
(60) Provisional application No. 60/411,398, filed on Sep. 16, 2002.
(60) Provisional application No. 60/412,341, filed on Sep. 20, 2002.

(51) Int. Cl. G01N 33/53; G06F 19/00; G01N 33/48; G01N 33/50

(52) U.S. Cl. 435/7.1; 702/19

(57) ABSTRACT

The present invention provides methods of designing ligands that specifically bind to target molecules by testing a set of distinct compounds to identify compound that bind to member of a molecular family of interest. Those compounds that show binding activity can be used as a starting point for ligand design. Unlike previous methods, even compounds that bind with only low affinity are of interest and can be used. Common chemical structures of binding molecules including low affinity binding molecules are identified, thereby providing molecular scaffolds. Co-crystals of the protein and the molecular scaffolds can be formed and analyzed by X-ray crystallography to determine the orientation of the scaffold compounds at the binding site of the molecule. Using the orientation information on a scaffold at the binding site of the molecule, chemically tractable structures of the scaffold are identified that are modified to provide ligands that bind to the target molecule with altered binding affinity or specificity to a target molecule or to members of a molecular family. New and useful ligands can therefore be designed in a rational manner and with a minimum investment of resources using the methods provided.

### RELATED APPLICATIONS

This application claims the benefit of U.S. Provisional Application No. 60/360,651, filed Feb. 28, 2002; U.S. Provisional Application No. 60/437,929, filed Jan. 2, 2003; U.S. Provisional Application No. 60/411,398, filed Apr. 16, 2002; U.S. Provisional Application No. 60/412,341, filed Sep. 20, 2002; and Ibrahim, et al., Provisional Application Number (not yet assigned), filed Feb. 28, 2003, entitled PYK2 CRYSTAL STRUCTURE AND USES, all of which are incorporated herein by reference in their entireties, including drawings.

### FIELD OF THE INVENTION

The present application relates to methods for designing ligands that bind to target molecules. The methods are useful in the design of drug products with superior properties.

### BACKGROUND OF THE INVENTION

The information provided below is intended to assist in understanding the invention and does not constitute an admission that any of that information provided or references cited is prior art.

The design of new drugs has traditionally focused on the random design of potential drug candidates and the mass screening of those candidates for a desired property. After generating compounds using combinatorial chemistry and performing high throughput screening on thousands of compounds, a particular compound can be identified that has a desired property, which then goes on to become a more serious candidate for drug development. Most existing drugs have been discovered by this random screening approach, but these methods are highly inefficient.

There have also been attempts to use various methods of rational drug design. In most cases, such methods utilize structural information about the target molecule and attempt to design candidate drug molecules that will fit the target structure, either using de novo design or starting with a screening hit.

Yayon, PCT/IL01/00871, WO 02/24722 describes a method for designing candidate agonists and antagonists of the FGFR3, by using computer modeling with a crystal structure of the target to construct candidate agonist or antagonist compounds. The compounds are synthesized and their biological activity is assayed. (See, e.g., p.7, lines 14-20.)

Smit, PCT/EP01/01457, WO 01/58951 provides a set of atomic coordinates for acetylcholine binding protein, and states that the crystal structure “can be used to generate 3D models of the extracellular ligand-binding domain of ligand-gated ion channels, and thus for screening of drugs that act on these ion channels.” (Abstract; Table 1, p,72 ff.)

Scanlan et al., PCT/Us98/25296, W) 99/26966 describes atomic coordinates for crystals of rat and human thyroid hormone receptor (TR) ligand binding domain, and indicates that the structural information can be used to design synthetic ligands. (p.9)

Nienaber, U.S. Pat. No. 6,297,021, issued Oct. 2, 2001, states that “crystallography can be used to screen and identify compounds that are not know ligands of a target biolomecule for their ability to bind the target.” The method involves “obtaining a crystal of a target biomolecle, exposing the target to one or more test samples that are potential ligands of the target; and determining whether a ligand/biomolecule complex is formed. Structural information from the ligand/receptor complexes found are used to design new ligands that bind tighter, bind more specifically, have better biological activity or have better safety profile than known ligands.” (Col. 1, line 57 to col. 8, lines 5.))

### SUMMARY OF THE INVENTION

The present invention provides methods of designing ligands that bind to target molecules by identifying compounds that bind, even weakly, to a target molecule or multiple members of a molecular compound family. Identification of such binding compounds is used to identify common core physical and structural features of binding compounds, thereby providing parent compounds for further ligand development. Binding compounds that bind to target with low or very low affinity and/or bind to a plurality of targets in a molecular family can be designated as molecular scaffolds. Such molecular scaffolds thus represent a minimal or neat minimal binding compound. Typically a large number of derivative binding compounds can be identified that share a common structural core (i.e., scaffold core) with a molecular scaffold, thus constituting a scaffold group or set.

Using analysis of co-crystals of one or more binding compounds (e.g., exemplary molecular scaffolds), the orientations (positioning) of the binding compounds at the binding site is determined, generally in three dimensions. Such analysis typically involves X-ray crystallography and/or nuclear magnetic resonance (NMR). The orientation information is used to guide the synthesis and/or selection of ligands that are modified from the initial binding compounds or molecular scaffolds, preferably by modification at chemically tractable locations on the molecule. Such modification provides compounds with altered specificity and/or binding affinity as compared to the original binding compounds or scaffolds.

This method of using molecular scaffolds as the basis for chemical modification to provide further compounds with altered binding and other activity properties allows focus on low molecular weight binding compounds, preferably about 150 to 350 Daltons. Focusing on small binding compounds provides molecular scaffolds that can be used for the develop of ligands for a variety of different molecules.

It is often difficult to identify compounds of this size with required specificity and activity properties using conventional compound screening. Thus, contrasted with conventional screening practice, the present invention removes binding compound potency or binding affinity as a primary criterion. Instead, all compounds showing activity, even quite low activity, can be utilized to lead to the synthesis of new ligands.

In addition, selecting compounds that are active across multiple members of a protein family or other target family, allows the identification of a scaffold, or preferably multiple different scaffolds, that can be used for development of ligands specific for a particular target, or targeting a subset of particular targets in the family.

Therefore, in a first aspect, the present invention provides methods of designing a ligand that binds to at least one target molecule that is a member of a molecular family. The methods involve identifying compounds as molecular scaffolds that bind to the binding site of the target molecule (or a plurality of target molecules in the molecular family) and produce a detectable signal in a binding assay and/or activity assay that indicates statistically significant binding to target at a confidence level of at least 90%, preferably at least 95, 97, 98, 99% or greater confidence level; determining the orientation of the molecular scaffolds at the binding site of the target molecule to identify chemically tractable structures of the scaffolds that, when modified, alter the binding affinity or binding specificity between the scaffold and the target molecule; and synthesizing or otherwise obtaining a ligand wherein one or more chemically tractable structures of the molecular scaffold is modified to provide a ligand that binds to the target molecule with altered binding affinity or binding specificity. The background signal of the binding assay can be measured under standard conditions, and a library of distinct compounds can be assayed for binding to a binding site of the target molecule.

In various embodiments, the methods include isolating co-crystals of one or more molecular scaffolds bound to the target molecule, and determining the orientation of the molecular scaffold by performing X-ray crystallography on the co-crystals. Common chemical structures of the molecular scaffolds can be identified and the molecular scaffolds placed into groups based on having at least one common chemical structure. The orientation of the molecular scaffolds at the binding site of the target molecule can thus be determined for a representative compound from a selected group or from a plurality of groups, e.g., at least 2, 4, 6, 8, 10, 20, 50, 100, 200, 300, 400, 500, 600, 800, 1000, or even more groups. In preferred embodiments, when modified, the resulting ligand can bind to the target molecule with greater binding affinity or greater binding specificity or both than the molecular scaffold. However, in some cases, it may be desirable to select a ligand that binds with a lower affinity, e.g., to enhance turnover or to prevent full inhibition of a type of target molecule. It can also be advantageous to co-crystallize ligands and determine their orientation at the binding site for performing designing further modified compounds as further ligands.

In other embodiments, the orientation of the molecular scaffold can be determined by nuclear magnetic resonance or NMR combined with crystallography results to determine orientation.

In preferred embodiments, each of a plurality of distinct compounds is assayed for binding to a plurality of members of the molecular family. In preferred embodiments, the distinct compounds have a molecular weight of from about 100 to about 350 daltons, or more preferably from about 150 to about 350 daltons or from 150 to 300 daltons, or from 200 to 300 daltons.

In preferred embodiments, the target molecule is a protein and the molecular family a protein family. In preferred embodiments, the protein is an enzyme or receptor. The protein family can be, for example, a protein kinase family or sub-family (e.g., tyrosine protein kinases, serine/threonine protein kinases, MAP protein kinases, cyclin-dependent protein kinases), proteases, and phosphatases (e.g., protein tyrosine phosphatases and serine/threonine phosphoprotein phosphatases).

The distinct compounds of the invention can be of a variety of structures. In some embodiments, the distinct compounds can have a ring structure, either a carbocyclic or heterocyclic ring, such as for example, a benzyl ring, an pyrrole, imidazole, pyridine, purine, or any ring structure.

In various embodiments, a compound or compounds binds with extremely low affinity, very low affinity, low affinity, moderate affinity, or high affinity; at least about 5% of the binding compounds bind with low affinity (and/or has low activity), or at least about 10%, 15%, or 20% of the compounds bind with low affinity (or very low or extremely low). After the identification of common chemical structures of the distinct compounds that bind, the compounds can be grouped into classes based on common chemical structures and at least one representative compound from at least one, or preferably a plurality, of the classes selected for performing orientation determination, e.g., by X-ray crystallography and/or NMR analysis.

In selecting the distinct compounds for assay in the present invention, the selection can be based on various criteria appropriate for the particular application, such as molecular weight, clogP (or other method of assessing lipophilicity), Polar Surface Area (PSA) (or other indicator of charge and polarity or related properties), and the number of hydrogen bond donors and acceptors. Compounds can also be selected using the presence of specific chemical moieties which, based on information derived from the molecular family, might be indicated as having predisposing some affinity for members of the family. Compounds with highly similar structures and/or properties can be identified and grouped using computational techniques to facilitate the selection of a representative subset of the group. As indicated above, in preferred embodiments, the molecular weight is from about 150 to about 350 daltons, more preferably from 150 to 300 daltons. The clog P is preferably less than 2, the number of hydrogen bond donors and acceptors is preferably less than 5 and the PSA less than 100. Compounds can be selected that include chemical structures of drugs having acceptable pharmacalogical properties and/or lacking chemical strutures that are know to result in undesirable pharmacological properties, e.g., excessive toxicity and lack of solubility.

In some embodiments, the assay is an enzymatic assay, and the number of groups of molecular scaffolds formed can conveniently be about 500. The binding of the ligand to the target molecule can cause a specific biochemical effect due to the inhibition of an enzyme. In some embodiments, the assay is a competition assay, e.g., a binding competition assay. Cell-based assays can also be used. As indicated above, compounds can be used that have low, very low, or extremely low activity in a biochemical or cell-based assay.

The modification of a molecular scaffold can be the addition, subtraction, or substitution of a chemical group. The modification may desirably cause the scaffold to be actively transported to or into particular cells and/or a particular organ. In various embodiments, the modification of the compound includes the addition or subtraction of a chemical atom, substituent or group, such as, for example, a hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (e.g., forming a ketone, ether or N-oxide), and a sulphur atom (e.g., forming a thiol, thione, sulfonamide or di-alkylsulfoxide (sulfone)).

In preferred embodiments, the information provided by performing X-ray crystallography on the co-crystals is provided to a computer program, wherein the computer program provides a measure of the interaction between the molecular scaffold and the protein and a prediction of changes in the interaction between the molecular scaffold and the protein that result from specific modifications to the molecular scaffold, and the molecular scaffold is chemically modified based on the prediction of the biochemical result. The computer program can provide the prediction based on a virtual assay such as, for example, virtual docking of the compound to the protein, shape-based matching, molecular dynamics simulations, free energy perturbation studies, and similarity to a three-dimensional pharmacophore. A variety of such programs are well-known in the art.

Chemical modification of a chemically tractable structure can result, or be selected to provide one or more physical changes, e.g., to result, in a ligand that fills a void volume in the protein-ligand complex, or in an attractive polar interaction being produced in the protein-ligand complex. The modification can also result in a sub-structure of the ligand being present in a binding pocket of the protein binding site when the protein-ligand complex is formed. After common chemical structures of the compounds that bind are identified, the compounds can be grouped based on having a common chemical sub-structure and a representative compound from each group (or a plurality of groups) can be selected for co-crystallization with the protein and performance of the X-ray crystallography. The X-ray crystallography is preferably performed on the co-crystals under at least 20, 30, 40, or 50 distinct environmental conditions, or more preferably under about 96 distinct environmental conditions. The X-ray crystallography and the modification of a chemically tractable structure of the compound can each be performed a plurality of times, e.g., 2, 3, 4, or more rounds of crystallization and modification.

Also in certain embodiments, one or molecular scaffolds are selected to have binding to a plurality of members of a molecular compound family, e.g., a protein family.

The method can also include the identification of conserved residues in a binding site(s) of a target protein that interact with a molecular scaffold, ligand or other binding compound. Conserved residues can, for example, be identified by sequence alignment of different members of a family, and identifying binding site residues that are the same or at least similar between multiple member of the family. Interacting residues can be characterized as those within a selected distance from the binding compound(s), e.g., 3, 3.5, 4, 4.5, or 5 angstroms.

In a related aspect, the present invention provides a method for designing a ligand that binds to at least one target molecule by assaying a plurality of distinct compounds for binding to a binding site of the target molecule, where at least one compound binds with low affinity. Generally the target molecule is a member of a molecular family. One or more compounds that bind to the binding site of the target molecule are identified as molecular scaffolds, and the orientation of the one or more molecular scaffolds at the binding site of the target molecule determined to identify chemically tractable structures of the scaffolds that, when modified, alter the binding affinity or binding specificity between the scaffold and the target molecule. A ligand or a plurality of ligands can be synthesized wherein one or more of the chemically tractable structures of the molecular scaffold is modified to provide a ligand or ligands that bind to the target molecule with altered binding affinity or binding specificity.

Particular embodiments include those described for the first aspect above.

The invention also provides a method to identify properties that a likely binding compound will possess, thereby allowing, for example, more efficient selection of compounds for structure activity relationship determinations and/or for selection for screening. Thus, another aspect concerns a method for identifying binding characteristics of a ligand of a target protein, by identifying at least one conserved interacting residue in the target protein that interacts with at least two binding molecules; and identifying at least one common interaction property of those binding molecules with the conserved residue(s). The interaction property and location with respect to the structure of the binding compound defines the binding characteristic.

In various embodiments, the identification of conserved interacting residues involves comparing (e.g., by sequence alignment) a plurality of amino acid sequences in a protein family to which the target protein belongs and identifying binding site residues conserved in that protein family; identification of binding site residues by determining a co-crystal structure; identifying interacting residues (preferably conserved residues) within a selected distance of the binding compounds, e.g., 3, 3.5, 4, 4.5, or 5 angstroms; the interaction property involves hydrophobic interaction, charge-charge interaction, hydrogen bonding, charge-polar interaction, polar-polar interaction, or combinations thereof.

Another related aspect concerns a method for developing ligands for a target using a set of scaffolds. The method involves selected a target, selecting a molecular scaffold, or a compound from a scaffold group, from a set of at least 3 scaffolds or scaffold groups where each of the scaffolds or compounds from each scaffold group are known to bind to the target. In particular embodiments, the target is a protein, for example a kinase, a phosphatase, a hormone receptor, a phophodiesterase, or other target as described herein. The set of scaffolds or scaffold groups is at least 4, 5, 6, 7, 8, or even more scaffolds or scaffold groups.

Another aspect concerns a method for identifying structurally and energetically allowed sites on a binding compound for attachment of an additional component(s) by analyzing the orientation of the binding compound(s) in a target binding site (e.g., by analyzing co-crystal structures), thereby identifying accessible sites on the compound for attachment of the separate component.

In various embodiments, the method involves calculating the change in binding energy on attachment of the separate component at one or more of the accessible sites; the orientation is determined by co-crystallography; the separate component includes a linker, a label such as a fluorophore, a solid phase material such as a gel, bead, plate, chip, or well.

In a related aspect, the invention provides a method for attaching a binding compound to an attachment component(s), by identifying energetically allowed sites for attachment of a said attachment component on a binding compound (e.g., as described for the preceding aspect), and attaching the compound or derivative thereof to the attachment component(s) at the energetically allowed site(s).

In various embodiments, the attachment component is a linker (which can be a traceless linker) for attachment to a solid phase medium, and the method also involves attaching the compound or derivative to a solid phase medium through the linker attached at the energetically allowed site; the binding compound or derivative thereof is synthesized on a linker attached to the solid phase medium; a plurality of compounds or derivatives are synthesized in combinatorial synthesis; the attachment of the compound(s) to the solid phase medium provides an affinity medium

A related aspect concerns a method for making an affinity matrix for a target molecule, where the method involves identifying energetically allowed sites on a target binding compound for attachment to a solid phase matrix; and attaching the target binding compound to the solid phase matrix through the energetically allowed site.

Various embodiments are as described for attachment of a separate component above; identifying energetically allowed sites for attachment to a solid phase matrix is performed for at least 5, 10, 20, 30, 50, 80, or 100 different compounds; identifying energetically allowed sites is performed for molecular scaffolds or other target binding compounds having different core ring structures.

In yet another aspect, the invention provides a modulator of a target molecule, e.g., an inhibitor, identified by the scaffold-based drug discovery methods (i.e., ligand development) described herein. In particular embodiments, the inhibitor is directed to an enzyme, e.g., a kinase, phosphatase, phosphodiesterase, methyltransferase, or other enzyme target described herein.

As used herein in connection with the design or development of ligands, the term “bind” and “binding” and like terms refer to a non-convalent energetically favorable association between the specified molecules (i.e., the bound state has a lower free energy than the separated state, which can be measured calorimetrically). For binding to a target, the binding is at least selective, that is, the compound binds preferentially to a particular target or to members of a target family at a binding site, as compared to non-specific binding to unrelated proteins not having a similar binding site. For example, BSA is often used for evaluating or controlling for non-specific binding. In addition, for an association to be regarded as binding, the decrease in free energy going from a separated state to the bound state must be sufficient so that the association is detectable in an biochemical assay suitable for the molecules involved.

By “assaying” is meant the creation of experimental conditions and the gathering of data regarding a particular result of the experimental conditions. For example, enzymes can be assayed based on their ability to act upon a detectable substrate. Likewise, for example, a compound or ligand can be assayed based on its ability to bind to a particular target molecule or molecules and/or to modulate an activity of a target molecule.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

When a decision is described as “based on” particular criteria, it is meant that the criteria selected are parameters of the decision and guide its outcome. A substantial change in the parameters is likely to result in a change in the decision.

By “binding site” is meant an area of a target molecule to which a ligand can bind non-covalently. Binding sites embody particular shapes and often contain multiple binding pockets present within the binding site. The particular shapes are often conserved within a class of molecules, such as a molecular family. Binding sites within a class also can contain conserved structures such as, for example, chemical moieties, the presence of a binding pocket, and/or an electrostatic charge at the binding site or some portion of the binding site, all of which can influence the shape of the binding site.

By “binding pocket” is meant a specific volume within a binding site. A binding pocket is a particular space within a binding site at least partially bounded by target molecule atoms. Thus a binding pocket is a particular shape, indentation, or cavity in the binding site. Binding pockets can contain particular chemical groups or structures that are important in the non-covalent binding of another molecule such as, for example, groups that contribute to ionic, hydrogen bonding, van der Waals, or hydrophobic interactions between the molecules.

By “chemical structure” or “chemical substructure” is meant any definable atom or group of atoms that constitute a part of a molecule. Normally, chemical substructures of a scaffold or ligand can have a role in binding of the scaffold or ligand to a target molecule, or can influence the three-dimensional shape, electrostatic charge, and/or conformational properties of the scaffold or ligand.

By “orientation”, in reference to a binding compound bound to a target molecule is meant the spatial relationship of the binding compound and at least some of its consitituent atoms to the binding pocket and/or atoms of the target molecule at least partially defining the binding pocket.

In the context of target molecules in the present invention, the term “crystal” refers to an ordered complex of target molecule, such that the complex produces an X-ray diffraction pattern when placed in an X-ray beam. Thus, a “crystal” is distinguished from a disordered or partially ordered complex or aggregate of molecules that do not produce such a diffraction pattern. Preferably a crystal is of sufficient order and size to be useful for X-ray crystallography. A crystal may be formed only of target molecule (with solvent and ions) or may be a co-crystal of more than one molecule, for example, as a co-crystal of target molecule and binding compound, and/or of a complex of proteins (such as a holoenzyme).

In the context of this invention, unless otherwise specified, by “co-crystals” is meant an ordered complex of the compound, molecular scaffold, or ligand bound non-covalently to the target molecule that produces a diffraction pattern when placed in an X-ray beam. Preferably the co-crystal is in a form appropriate for analysis by X-ray or protein crystallography. In preferred embodiments the target molecule-ligand complex can be a protein-ligand complex.

By “clog P” is meant the calculated log P of a compound, “P” referring to the partition coefficient of the compound between a lipophilic and an aqueous phase, usually between octanol and water.

By “chemically tractable structures” is meant chemical structures, sub-structures, or sites on a molecule that can be covalently modified to produce a ligand with a more desirable property. The desirable property will depend on the needs of the particular situation. The property can be, for example, that the ligand binds with greater affinity to a target molecule, binds with more specificity, or binds to a larger or smaller number of target molecules in a molecular family, or other desirable properties as needs require.

By “designing a ligand,” “preparing a ligand,” “discovering a ligand,” and like phrases is meant the process of considering relevant data (especially, but not limited to, any individual or combination of binding data, X-ray co-crystallography data, molecular weight, clogP, and the number of hydrogen bond donors and acceptors) and making decisions about advantages that can be achieved with resort to specific structural modifications to a molecule, and implementing those decisions. This process of gathering data and making decisions about structural modifications that can be advantageous, implementing those decisions, and determining the result can be repeated as many times as necessary to obtain a ligand with desired properties.

By “docking” is meant the process of attempting to fit a three-dimensional configuration of a binding pair member into a three-dimensional configuration of the binding site or binding pocket of the partner binding pair member, which can be a protein, and determining the extent to which a fit is obtained. The extent to which a fit is obtained can depend on the amount of void volume in the resulting binding pair complex (or target molecule-ligand complex). The configuration can be physical or a representative configuration of the binding pair member, e.g., an in silico representation or other model.

By “ligand” is meant a molecular scaffold that has been chemically modified at one or more chemically tractable structures to bind to the target molecule with altered or changed binding affinity or binding specificity relative to the molecular scaffold. The ligand can bind with a greater specificity or affinity for a member of the molecular family relative to the molecular scaffold. A ligand binds non-covalently to a target molecule, which can preferably be a protein or enzyme.

By binding with “low affinity” is meant binding to the target molecule with a dissociation constant (k_{d}) of greater than 1 μM under standard conditions. In particular cases, low affinity binding is in a range of 1 μM-10 mM, 1 μM-1 mM, 1 μM -500 μM, 1 μM-200 μM, 1 μM-100 μM. By binding with “very low affinity” is meant binding with a k_{d} of above about 100 μM under standard conditions, e.g., in a range of 100 μM-1 mM, 100 μM-500 μM, 100 μM-200 μM. By binding with “extremely low affinity” is meant binding at a k_{d} of above about 1 mM under standard conditions. By “moderate affinity” is meant binding with a k_{d} of from about 200 nM to about 1 μM under standard conditions. By “moderately high affinity” is meant binding at a k_{d} of from about 1 nM to about 200 nM. By binding at “high affinity” is meant binding at a k_{d} of below about 1 nM under standard conditions. For example, low affinity binding can occur because of a poorer fit into the binding site of the target molecule or because of a smaller number of non-covalent bonds, or weaker covalent bonds present to cause binding of the scaffold or ligand to the binding site of the target molecule relative to instances where higher affinity binding occurs. The standard conditions for binding are at pH 7.2 at 37° C. for one hour. For example, 100 μl/well can be used in HEPES 50 mM buffer at pH 7.2, NaCl 15 mM, ATP 2 μM, and bovine serum albumin 1 μg/well, 37° C. for one hour.

Binding compounds can also be characterized by their effect on the activity of the target molecule. Thus, a “low activity”-compound has an inhibitory concentration (IC₅₀) or excitation concentration (EC₅₀) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured.

By “molecular scaffold” or “scaffold” is meant a small target binding molecule to which one or more additional chemical moieties can be covalently attached, modified, or eliminated to form a plurality of molecules with common structural elements. The moieties can include, but are not limited to, a halogen atom, a hydroxyl group, a methyl group, a nitro group, a carboxyl group, or any other type of molecular group including, but not limited to, those recited in this application. Molecular scaffolds bind to at least one target molecule with low or very low affinity and/or bind to a plurality of molecules in a target family (e.g., protein family), and the target molecule is preferably an enzyme, receptor, or other protein. Preferred characteristics of a scaffold include molecular weight of less than about 350 daltons; binding at a target molecule binding site such that one or more substituents on the scaffold are situated in binding pockets in the target molecule binding site; having chemically tractable structures that can be chemically modified, particularly by synthetic reactions, so that a combinatorial library can be easily constructed; having chemical positions where moieties can be attached that do not interfere with binding of the scaffold to a protein binding site, such that the scaffold or library members can be modified to form ligands, to achieve additional desirable characteristics, e.g., enabling the ligand to be actively transported into cells and/or to specific organs, or enabling the ligand to be attached to a chromatography column for additional analysis. Thus, a molecular scaffold is a small, identified target binding molecule prior to modification to improve binding affinity and/or specificity, or other pharmacalogic properties.

The term “scaffold core” refers to the core structure of a molecular scaffold onto which various substituents can be attached. Thus, for a number of scaffold molecules of a particular chemical class, the scaffold core is common to all the scaffold molecules. In many cases, the scaffold core will consist of or include one or more ring structures.

The term “scaffold group” refers to a set of compounds that share a scaffold core and thus can all be regarded as derivatives of one scaffold molecule.

In particular embodiments, the a scaffold or scaffold group for use in this invention is not a quinazoline; not a purine; not an oxindole; not a pyrimidine.

By “molecular family” is meant groups of molecules classed together based on structural and/or functional similarities. Examples of molecular families include proteins, enzymes, polypeptides, receptor molecules, oligosaccharides, nucleic acids, DNA, RNA, etc. Thus, for example, a protein family is a molecular family. Molecules can also be classed together into a family based on, for example, homology. The person of ordinary skill in the art will realize many other molecules that can be classified as members of a molecular family based on similarities in chemical structure or biological function.

By “protein-ligand complex” or “co-complex” is meant a protein and ligand bound non-covalently.

By “protein” is meant a polymer of amino acids. The amino acids can be naturally or non-naturally occurring. Proteins can also contain adaptations, such as being glycosylated, phosphorylated, or other common modifications.

By “protein family” is meant a classification of proteins based on structural and/or functional similarities. For example, kinases, phosphatases, proteases, and similar groupings of proteins are protein families. Proteins can be grouped into a protein family based on having one or more protein folds in common, a substantial similarity in shape among folds of the proteins, homology, or based on having a common function. In many cases, smaller families will be specified, e.g., tyrosine kinases, serine/threonine kinases, and the like.

“Protein folds” are 3-dimensional shapes exhibited by the protein and defined by the existence, number, and location in the protein of alpha helices, beta-sheets, and loops, i.e., the basic secondary structures of protein molecules. Folds can be, for example, domains or partial domains of a particular protein.

By “ring structure” is meant a molecule having a chemical ring or sub-structure that is a chemical ring. In most cases, ring strutures will be carbocyclic or heterocyclic rings. The chemical ring may be, but is not limited to, a benzyl ring, aryl ring, pyrrole ring, imidazole, pyridine, purine, or any ring structure.

By “specific biochemical effect” is meant a therapeutically significant biochemical change in a biological system causing a detectable result. This specific biochemical effect can be, for example, the inhibition or activation of an enzyme, the inhibition or activation of a protein that binds to a desired target, or similar types of changes in the body's biochemistry. The specific biochemical effect can cause alleviation of symptoms of a disease or condition or another desirable effect. The detectable result can also be detected through an intermediate step.

By “standard conditions” is meant conditions under which an assay is performed to obtain scientifically meaningful data. Standard conditions are dependent on the particular assay, and can be generally subjective. Normally the standard conditions of an assay will be those conditions that are optimal for obtaining useful data from the particular assay. The standard conditions will generally minimize background signal and maximize the signal sought to be detected.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667$$

By a “set” of compounds is meant a collection of compounds. The compounds may or may not be structurally related.

In the context of this inveniton, by “target molecule” is meant a molecule that a compound, molecular scaffold, or ligand is being assayed for binding to. The target molecule has an activity that binding of the molecular scaffold or ligand to the target molecule will alter or change. The binding of the compound, scaffold, or ligand to the target molecule can preferably cause a specific biochemical effect when it occurs in a biological system. A “biological system” includes, but is not limited to, a living system such as a human, animal, plant, or insect. In most but not all cases, the target molecule will be a protein or nucleic acid molecule.

By “pharmacophore” is meant a representation of molecular features that are considered to be responsible for a desired activity, such as interacting or binding with a receptor. A pharmacophore can include 3-dimensional (hydrophobic groups, charged/ionizable groups, hydrogen bond donors/acceptors), 2D (substructures), and ID (physical or biological) properties.

As used herein in connection with numerical values, the terms “approximately” and “about” mean 110% of the indicated value.

Additional embodiments will be apparent from the Detailed Description of the Invention and from the claims.

### BRIEF DESCRIPTION OF THE DRAWINGS

FIG. 1 is a schematic diagram illustrating some characteristics of a good scaffold.

FIG. 2A illustrates binding of a scaffold in a kinase homology surface. FIG. 2B illustrates the interaction of a kinase inhibitor in a homology surface.

FIG. 3 is a schematic showing initial steps in scaffold-based drug discovery approach, combining biochemical screening including identification of low affinity hits and crystallization and structure determination of target protein.

FIG. 4 shows the application of co-crystallization, as well as filters for selection of advantageous scaffolds to use for ligand development.

FIG. 5 provides a schematic view of an exemplary embodiment of the present invention. By assaying a large number of compounds for binding activity to members of a protein family, about 400 broadly acting “hits” or molecular scaffold embodiments are obtained. The orientations of at least some of these molecular scaffold compounds at the binding site of the target molecule are determined, and a plurality of the scaffold compounds are modified at chemically tractable structures, resulting in a smaller number of lead compounds with a greater specificity and/or greater affinity, directed to one or more respective targets that are members of a protein family.

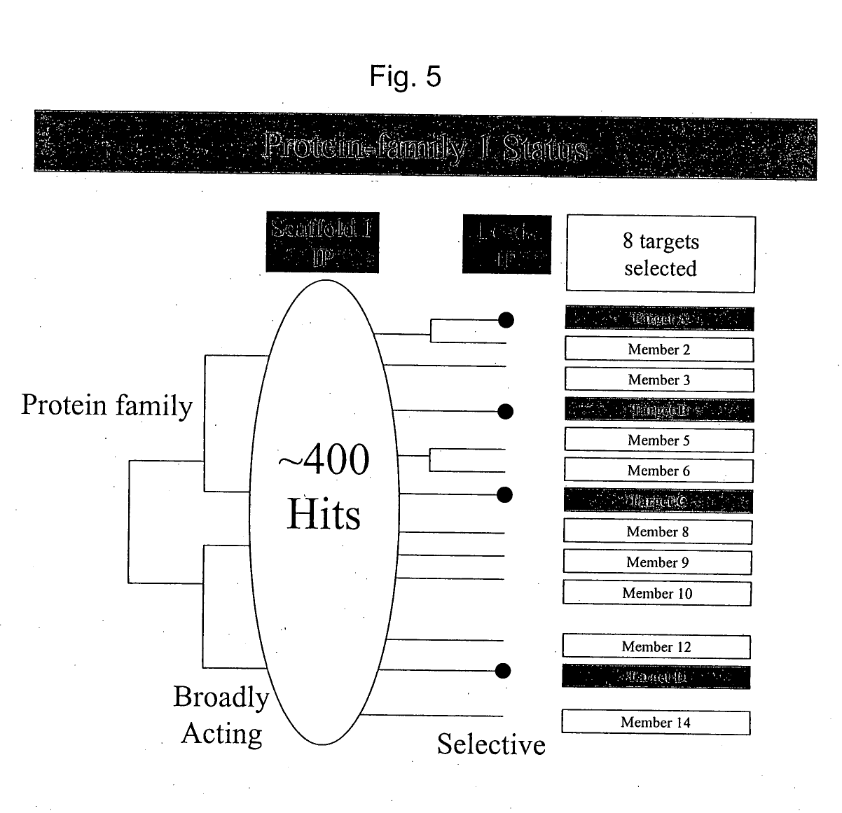

FIG. 6 illustrates a binding site surface with a molecular scaffold bound thereto, and illustrates that a modification of the scaffold can be performed to fill void volume at the target molecule-scaffold co-complex, or to eliminate a sub-molecular barrier to binding and provide greater access of the scaffold to the binding site, thereby resulting in a ligand that binds with greater affinity and/or greater specificity.

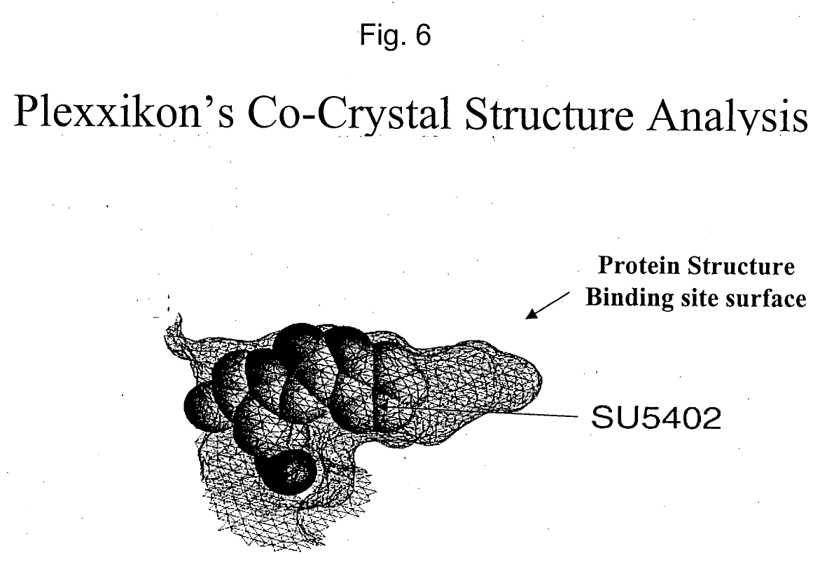

FIG. 7 illustrates a scaffold (hydrogens not shown) bound in PIM-1 binding site (panel A), and a derivative compound bound at the same site with an added substituent making a hydrophobic interaction with a nearby hydrophobic target surface (panel B).

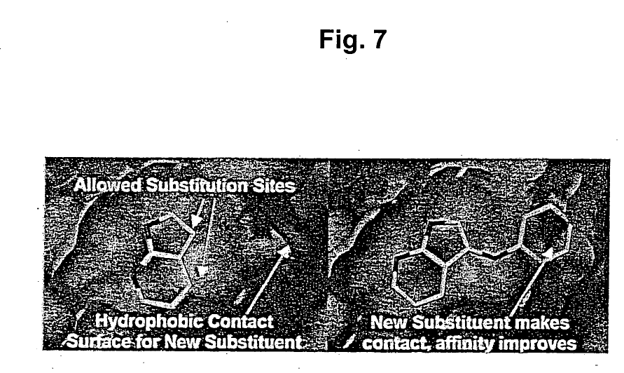

FIG. 8 provides an illustration of a scaffold at the binding site of a target compound. 5a illustrates a scaffold with broad binding activity, binding to HCK; and 5b and 5c show how the scaffold can be modified to arrive at ligands that are specific for individual targets, CSK and Lyn, which are homologous to HCK. Thus, beginning with one molecular scaffold, a plurality of ligands can be designed specific for particular target molecules within a molecular family. The initial selection of ligands can also be utilized as molecular scaffolds providing bases for further modification and design of ligands.

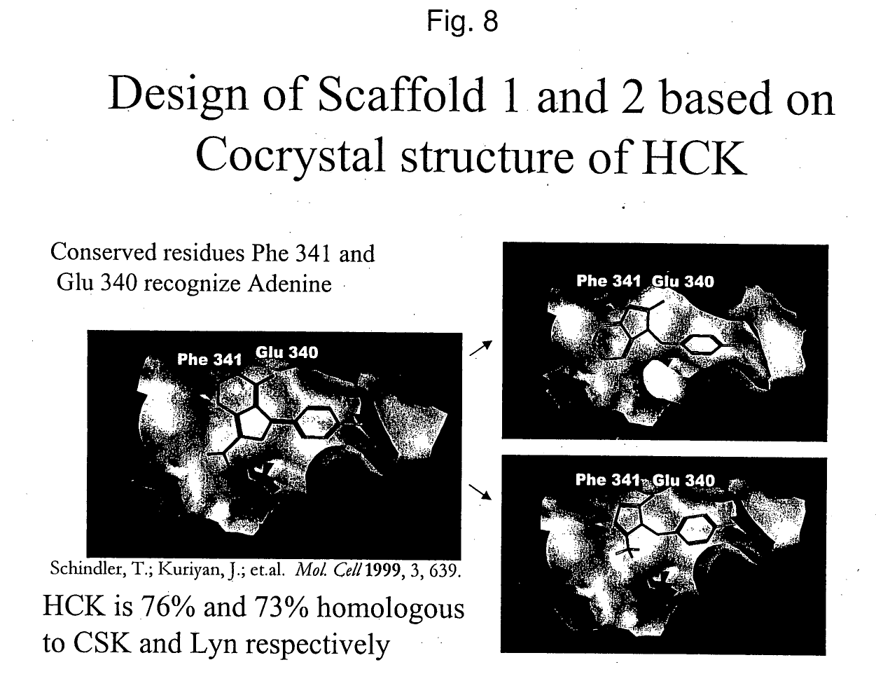

### DETAILED DESCRIPTION OF THE INVENTION

The Tables will first be briefly described.

Table 1 provides atomic coordinates for human PIM-1. In this table and in Table 4, the various columns have the following content, beginning with the left-most column:

ATOM: Refers to the relevant moiety for the table row.

Atom number: Refers to the arbitrary atom number designation within the coordinate table.

Atom Name: Identifier for the atom present at the particular coordinates.

Chain ID: Chain ID refers to one monomer of the protein in the crystal, e.g., chain “A”, or to other compound present in the crystal, e.g., HOH for water, and L for a ligand or binding compound. Multiple copies of the protein monomers will have different chain Ids.

Residue Number: The amino acid residue number in the chain.

X, Y, Z: Respectively are the X, Y, and Z coordinate values.

Occupancy: Describes the fraction of time the atom is observed in the crystal. For example, occupancy=1 means that the atom is present all the time; occupancy=0.5 indicates that the atom is present in the location 50% of the time.

B-factor: A measure of the thermal motion of the atom.

Element: Identifier for the element.

Table 2 provides atomic coordinates for PIM-1 with AMP-PNP in the binding site. Table entries are as for Table 1.

Table 3 provides an alignment of kinase domains of several PIM kinases, including human PIM-1, PIM-2, and PIM-3 as well as PIM kinases from other species.

Table 4 provides atomic coordinates for PYK2 with (5′-adenylylimidodiphosphate) AMPPNP in the binding site. Table entries are as for Table 1.

Table 5 provides an alignment of kinase domains for several kinases, including human PYK2, providing identification of residues conserved between various members of the set. The residue number is for PYK2.

Table 6 provides the nucleic acid and amino acid sequences for human PYK2 kinase domain.

Table 7 provides representative assay results for kinase activity of PYK2 kinase domain in the presence of ATP and in the presence of several ATP analogs.

The present invention provides methods for designing ligands active on particular biological targets, such as cellular enzymes and receptors. While such methods can be implemented in many ways, highly preferably the process utilizes molecular scaffolds. Such molecular scaffolds are low molecular weight molecules that bind with low or very low affinity to the target and typically have low or very low activity on that target and/or act broadly across families of target molecules.

The ability of a scaffold or other compound to act broadly across multiple members of a target family is advantageous in developing ligands. For example, a scaffold or set of scaffolds can serve as starting compounds for developing ligands with desired specificity or with desired cross-activity on a selected subset of members of a target family. Further, identification of a set of scaffolds that each bind with members of a target family provides an advantageous basis for selecting a starting point for ligand development for a particular target or subset of targets. In many cases, the ability of a scaffold to bind to and/or have activity on multiple members of a target family is related to active site or binding site homology that exists across the target family.

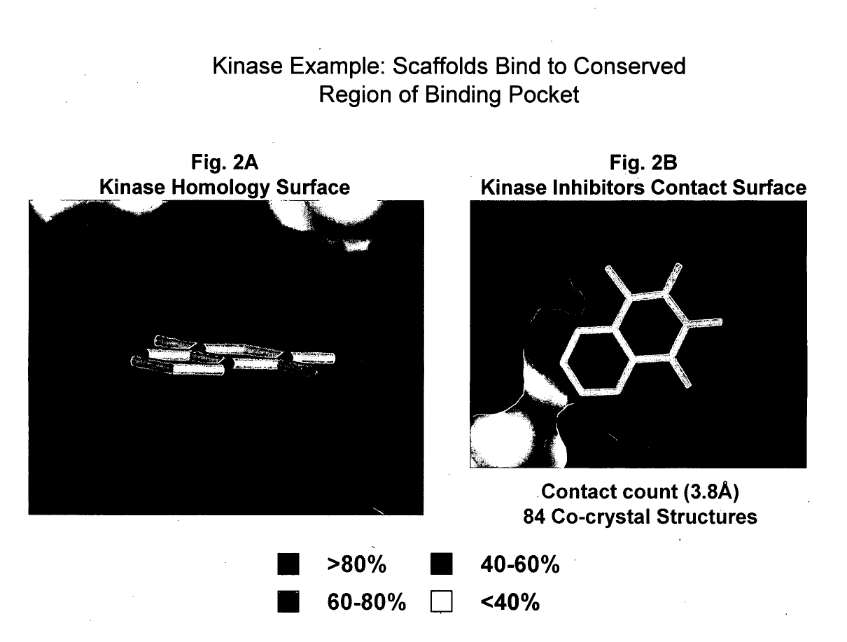

The relationship of the target homology and binding of a scaffold is illustrated in FIG. 2. As shown in the figure, a homology surface can be created, showing the presence of various levels of homology among kinase structures. A scaffold active across multiple members of the kinase family interacts with surfaces or residues of relatively high homology, i.e., binds to conserved regions of the binding pockets. Scaffolds that bind with multiple members can be modified to provide greater specificity or to have a particular cross-reactivity, e.g., by exploiting differences between target binding sites to provide specificity, and exploiting similarities to design in cross-reactivities. Adding substituents that provide attractive interactions with the particular target typically increases the binding affinity, often increasing the activity. The various parts of the ligand development process are described in more detail in following sections, but the following describes an advantageous approach for scaffold-based ligand development.

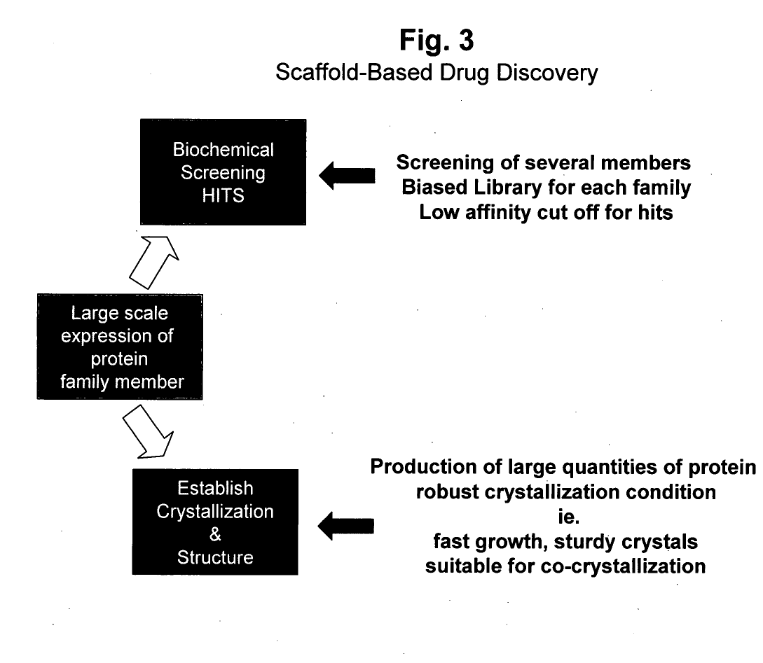

While understanding that scaffold-based ligand development (scaffold-based drug discovery) can be implemented in a variety of ways, the early steps of an illustrative approach is shown in FIG. 3. As shown, large scale expression of protein is useful to provide material for crystallization, co-crystallization, and biochemical screening (e.g., binding and activity assays). For crystallization, crystallization conditions can be established for apo protein and a structure determined from those crystals. For screening, preferably a biased library selected for the particular target family is screening for binding and/or activity on the target. Highly preferably a plurality of members from the target family are screened. Such screening, whether on a single target or on multiple members of a target family provides screening hits. Low affinity and/or low activity hits are selected. Such low affinity hits can either identify a scaffold molecule, or allow identification of a scaffold molecule by analyzing common features between binding molecules. Simpler molecules containing the common features can then be tested to determine if they retain binding and/or activity, thereby allowing identification of a scaffold molecule.

When multiple members of a particular target family are used for screening, the overlap in binding and/or activity of compounds can provide a useful selection for compounds that will be subjected to crystallization. For example, for 3 target molecules from a target family, if each target has about 500-200 hits in screening of a particular library, much smaller subsets of those hits will be common to any 2 of the 3 targets, and a still smaller subset will be common to all 3 targets, e.g., 100-300. In many cases, compounds in the subset common to all 3 targets will be selected for co-crystallography, as they provide the broadest potential for ligand development.

Once compounds for co-crystallography are selected, conditions for forming co-crystals are determined, allowing determination of co-crystal structure and the orientation of binding compound in the binding site of the target is determined by solving the structure (this can be highly assisted if an apo protein crystal structure has been determined or if the structure of a close homolog is available for use in a homology model. Preferably the co-crystals are formed by direct co-crystallization rather than by soaking the compound into crystals of apo protein.

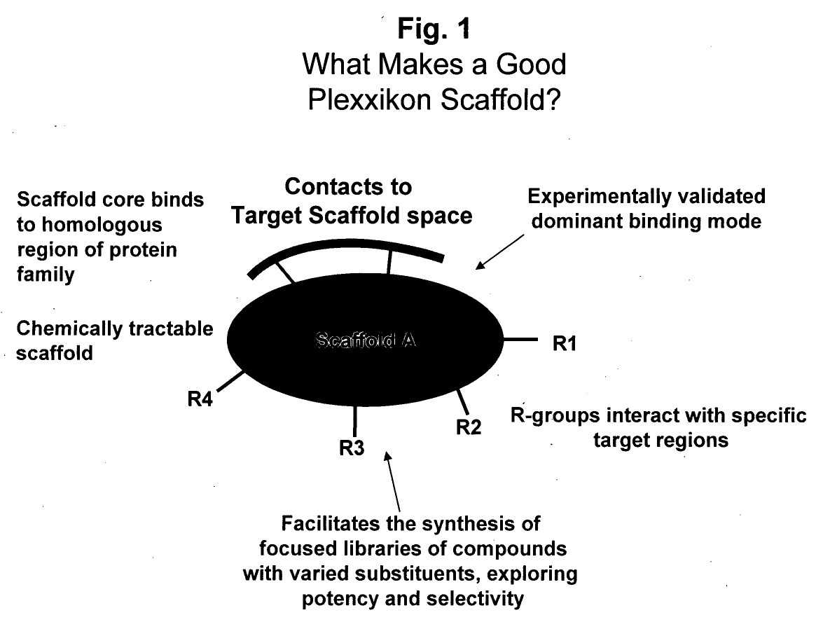

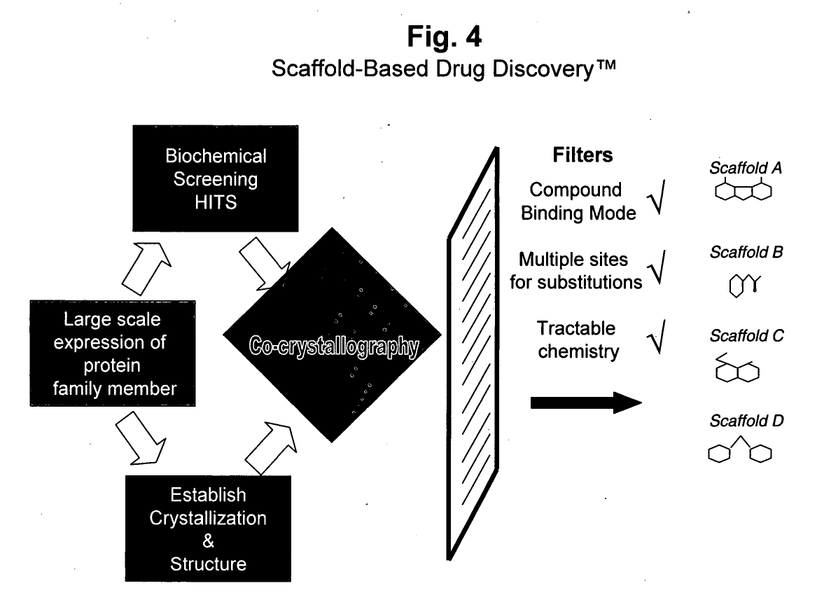

From the co-crystals and knowledge of the structure of the binding compounds, additional selection of scaffold (or other binding compounds can be made as shown in FIG. 4, by applying selection filters, e.g., for (1) binding mode, (2) multiple sites for substitution, and/or (3) tractable chemistry. A binding mode filter can, for example, be based on the demonstration of a dominant binding mode. That is, a scaffold or compounds of a scaffold group bind with a consistent orientation, preferably a consistent orientation across multiple members of a target family. Filtering scaffolds for multiple sites for substitution provides greater potential for developing ligands for specific targets due to the greater capacity for appropriately modifying the structure of the scaffold. Filtering for tractable chemistry also facilitates preparation of ligands derived from a scaffold because the synthetic paths for making derivative compounds are available. Some of the characteristics for a good scaffold are illustrated schematically in FIG. 1, which shows a schematic of a scaffold bound with a target. As indicated in the figure, such scaffolds facilitate preparation of focused libraries of derivative compounds with varied substituents.

Carrying out such a process of development provides scaffolds, preferably of divergent structure. This is illustrated by the four different strutures in FIG. 4 (the illustration of these structures does not mean that the compounds represented by those structures are actual scaffolds for any target).

In some cases, it may be impractical or undesirable to work with a particular target for some or all of the development process. For example, a particular target may be difficult to express, by easily degraded, or be difficult to crystallize. In these cases, a surrogate target from the target family. It is desirable to have the surrogate be as similar as possible to the desired target, thus a family member that has high homolgy in the binding site should be used, or the binding site can be modified to be more similar to that of the desired target, or part of the sequence of the desired target can be inserted in the family member replacing the corresponding part of the sequence of the family member.

Once one or more scaffolds are identified for a target family, the scaffolds can be used to develop multiple products directed at specific members of the family, or at specific subsets of family members. Thus, starting from a scaffold that acts on multiple member of the target family, derivative compounds (ligands) can be designed and tested that have increasing selectivity. In addition, such ligands are typically developed to have greater activity, and will also typically have greater binding affinity. In this process, starting with the broadly acting scaffold, ligands are developed that have improved selectivity and activity profiles, leading to identification of lead compounds for drug development, leading to drug candidates, and final drug products.

Scaffolds

Typically it is advantageous to select scaffolds (and/or compound sets or libraries for scaffold or binding compound identification) with particular types of characteristics, e.g., to select compounds that are more likely to bind to a particular target and/or to select compounds that have physical and/or synthetic properties to simplify preparation of derivatives, to be drug-like, and/or to provide convenient sites and chemistry for modification or synthesis.

Useful chemical properties of molecular scaffolds can include one or more of the following characteristics, but are not limited thereto: an average molecular weight below about 350 daltons, or between from about 150 to about 350 daltons, or from about 150 to about 300 daltons; having a clogP below 3; a number of rotatable bonds of less than 4; a number of hydrogen bond donors and acceptors below 5 or below 4; a Polar Surface Area of less than 100 Å²; binding at protein binding sites in an orientation so that chemical substituents from a combinatorial library that are attached to the scaffold can be projected into pockets in the protein binding site; and possessing chemically tractable structures at its substituent attachment points that can be modified, thereby enabling rapid library construction.

The term “Molecular Polar Surface Area (PSA)” refers to the sum of surface contributions of polar atoms (usually oxygens, nitrogens and attached hydrogens) in a molecule. The polar surface area has been shown to correlate well with drug transport properties, such as intestinal absorption, or blood-brain barrier penetration.

Additional useful chemical properties of distinct compounds for inclusion in a combinatorial library include the ability to attach chemical moieties to the compound that will not interfere with binding of the compound to at least one protein of interest, and that will impart desirable properties to the library members, for example, causing the library members to be actively transported to cells and/or organs of interest, or the ability to attach to a device such as a chromatography column (e.g., a streptavidin column through a molecule such as biotin) for uses such as tissue and proteomics profiling purposes.

A person of ordinary skill in the art will realize other properties that can be desirable for the scaffold or library members to have depending on the particular requirements of the use, and that compounds with these properties can also be sought and identified in like manner. Methods of selecting compounds for assay are known to those of ordinary skill in the art, for example, methods and compounds described in U.S. Pat. Nos. 6,288,234, 6,090,912, 5,840,485, each of which is hereby incorporated by reference in its entirety, including all charts and drawings.

In various embodiments, the present invention provides methods of designing ligands that bind to a plurality of members of a molecular family, where the ligands contain a common molecular scaffold. Thus, a compound set can be assayed for binding to a plurality of members of a molecular family, e.g., a protein family. One or more compounds that bind to a plurality of family members can be identified as molecular scaffolds. When the orientation of the scaffold at the binding site of the target molecules has been determined and chemically tractable structures have been identified, a set of ligands can be synthesized starting with one or a few molecular scaffolds to arrive at a plurality of ligands, wherein each ligand binds to a separate target molecule of the molecular family with altered or changed binding affinity or binding specificity relative to the scaffold. Thus, a plurality of drug lead molecules can be designed to individually target members of a molecular family based on the same molecular scaffold, and act on them in a specific manner.

Protein Families

Many classes or families of proteins are contemplated for use as target molecules in the present invention. For example protein kinases (e.g., tyrosine kinases and serine/threonine kinases), proteases (including serine proteases, cysteine proteases, metalloproteases (including matrix metalloproteases), and thrubedel proteases), phosphatases, nuclear hormone receptors, TNF receptors, G-protein coupled receptors, G-proteins, phospodiesterases, ATP or GTP cyclases, adaptor molecules (such as SH2, SH3, PTB, and WW), ATPases, GTPases, methyl transferases, acetyl transferases, sulfonyl transferases, dehydrogenases, CDK/cyclin exosite inhibitors, integrins, ligases (including ubiquitin ligases), bcl-2 homologs, NAD(P) oxoreductases, monooxygenases, integrases, cyclases, DNA and RNA polymerases, heperanases, helicases, ABC transporters, ion channels, immunoglobulins, and similar groups are contemplated. The protein classes or families are made based on the structural, chemical, or functional similarities of their members. For example, a protein class or family can be based on the common arrangement of secondary structure elements in three-dimensions that provides the core of a protein structure, such as the kinase fold, aspartyl protease fold, or hormone receptor fold. A protein class or family can also be represented by functional similarities, for example the cytokines.

Proteins that belong to protein families of interest can be obtained by any suitable methods. For example, standard methods of PCR cloning of cDNA libraries, or standard purification can be used to prepare nucleotide sequences encoding members of protein families. Plasmids for expressing protein can then be generated for expression of the protein in a suitable expressions system, such as (for example) E. coli, insect cell expression, or a commercially available “in vitro” translation and expression system (e.g., Roche Bioscience, Palo Alto, Calif.). These expression systems can then be used for obtaining protein for screening and co-crystallization studies. Proteins are typically expressed with a poly-histidine tag at either the N or C terminus of the protein sequence, which can greatly aid purification of the protein. The activity of the protein can initially be tested; for example kinases can be tested for phosphorylation activity, or nuclear receptors for hormone binding activity. Proteins showing activity can be screened against the compound collection and crystallized. In some cases, vectors with sequences encoding a designed protein will be publically available, such as from a commercial source or from a depository.

Protein expression can be carried out in any suitable system, for example, by growing cells transformed with the expression plasmid in either E. coli or insect cells using baculovirus. In the case of an “in vitro” expression system, a standard commercially available protein expression systems can be used (e.g., RTS500®, Roche Biosciences, Palo Alto, Calif.). Cells can then be lysed in buffers. A preferred buffer contains 20 mM Tris HCl pH 8.0, 200 mM NaCl, protease inhibitors, and 1-3% glycerol or propane diol. Batch purification of the protein can be done with the lysate from the cells using cobalt chelated beads, with the protein being isolated from the beads followed by a combination of ion exchange chromatography (such as Q-sepharose) or gel filtration (such as Sephadex 200). The buffers described above can be used or combinations of different buffer systems can be used that optionally contain other chemical additives.

G Protein Receptors

An exemplary protein family, also having sub-families is the G protein-coupled receptors (GPCRs). G protein-coupled receptors constitute an important family of validated drug targets within biomedical research; over half of approved drugs elicit their therapeutic effects by selectively addressing members of that target family. Many pharmacological drug companies are interested in the study of G-coupled proteins. It is possible to co-express a G-coupled protein receptor and its associated G-protein to study their pharmacological characteristics (Strosberg and Marullo, Functional expression of receptors in microorganisms. TIPS, 1992. 13: 95-98).

G protein coupled receptors (GPCRs) are reviewed by Sautel and Milligan, Molecular manipulation of G-protein-coupled receptors: a new avenue into drug discovery. (Review), Curr Med Chem 2000 889-96; Hibert et al., This is not a G protein-coupled receptor. (Review), Trends Pharmacol Sci 1993, 14:7-12; Wilson et al., Orphan G-protein-coupled receptors: the next generation of drug targets? (Review), Br J Pharmacol 1998, 125:1387-92; Roth et al., G protein-coupled receptor (GPCR) trafficking in the central nervous system: relevance for drugs of abuse. (Review), Drug Alcohol Depend 1998, 51:73-85; Ferguson and Caron, G protein-coupled receptor adaptation mechanisms. (Review), Semin Cell Dev Biol 1998, 9:119-27; Wank, G protein-coupled receptors in gastrointestinal physiology. I. CCK receptors: an exemplary family, Am J Physiol 1998, 274:G607-13; Rohrer and Kobilka, G protein-coupled receptors: functional and mechanistic insights through altered gene expression. (Review), Physiol Rev 1998, 78:35-52; and Larhammar et al., The receptor revolution—multiplicity of G-protein-coupled receptors. (Review), Drug Des Discov 1993, 9:179-88.

GPCR localization and regulation has been studied using Green Fluorescent Protein comprising fusion proteins Kallal and Benovic, Using green fluorescent proteins to study G-protein-coupled receptor localization and trafficking. (Review), Trends Pharmacol Sci 2000 21:175-80; and Ferguson, Using green fluorescent protein to understand the mechanisms of G-protein-coupled receptor regulation. (Review), Braz J Med Biol Res 1998, 31:1471-7; chimeric GPCRs, Milligan and Rees, Chimaeric G alpha proteins: their potential use in drug discovery. (Review), Erratum in: Trends Pharmacol Sci 1999 June; 20(6):252.

Members of the membrane protein gene superfamily of G-protein coupled receptors have been characterized as having seven putative transmembrane domains. The transmembrane domains are believed to represent transmembrane alpha-helices connected by extracellular or cytoplasmic loops. A functional G-protein is a trimer which consists of variable alpha subunit coupled to much more tightly-associated and constant beta and gamma subunits. A variety of ligands have been identified which function through GPCRs.

In general, binding of an appropriate ligand to a GPCR leads to the activation of the receptor. G-protein coupled receptors include a wide range of biologically active receptors, such as hormone, viral, growth factor and neuroreceptors. Typically, activation of a GPCR initiates the regulatory cycle of a corresponding G-protein. This cycle consists of GTP exchange for GDP, dissociation of the alpha and beta/gamma subunits, activation of the second messenger pathway by a complex of GTP and the alpha subunit of the G-protein, and return to the resting state by GTP hydrolysis via the innate GTP-ase activity of the G-protein alpha subunit A.

G-protein coupled receptors have been characterized as including these seven conserved hydrophobic stretches of about 20 to 30 amino acids, connecting at least eight divergent hydrophilic loops. The G-protein family of coupled receptors includes dopamine receptors which bind to neuroleptic drugs used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins and rhodopsins, odorant, cytomegalovirus receptors, and the like.

Most GPCRs havesingle conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 is also implicated in signal transduction.

Binding Assays

The methods of the present invention involve assays that are able to detect the binding of compounds to a target molecule at a signal of at least about three times the standard deviation of the background signal, or at least about four times the standard deviation of the background signal. The assays of the present invention can also include assaying compounds for low affinity binding to the target molecule. A large variety of assays indicative of binding are known for different target types and can be used for this invention. Compounds that act broadly across protein families are not likely to have a high affinity against individual targets, due to the broad nature of their binding. Thus, assays (e.g., as described herein) highly preferably allow for the identification of compounds that bind with low affinity, very low affinity, and extremely low affinity. Therefore, potency (or binding affinity) is not the primary, nor even the most important, indicia of identification of a potentially useful binding compound. Rather, even those compounds that bind with low affinity, very low affinity, or extremely low affinity can be considered as molecular scaffolds that can continue to the next phase of the ligand design process.

As indicated above, to design or discover scaffolds that act broadly across protein families, proteins of interest can be assayed against a compound collection or set. The assays can preferably be enzymatic or binding assays. In some embodiments it may be desirable to enhance the solubility of the compounds being screened and then analyze all compounds that show activity in the assay, including those that bind with low affinity or produce a signal with greater than about three times the standard deviation of the background signal. The assays can be any suitable assay such as, for example, binding assays that measure the binding affinity between two binding partners. Various types of screening assays that can be useful in the practice of the present invention are known in the art, such as those described in U.S. Pat. Nos. 5,763,198, 5,747,276, 5,877,007, 6,243,980, 6,294,330, and 6,294,330, each of which is hereby incorporated by reference in its entirety, including all charts and drawings.

In various embodiments of the assays at least one compound, at least about 5%, at least about 10%, at least about 15%, at least about 20%, or at least about 25% of the compounds can bind with low affinity. In many cases, up to about 20% of the compounds can show activity in the screening assay and these compounds can then be analyzed directly with high-throughput co-crystallography, computational analysis to group the compounds into classes with common structural properties (e.g., structural core and/or shape and polarity characteristics), and the identification of common chemical structures between compounds that show activity.

The person of ordinary skill in the art will realize that decisions can be based on criteria that are appropriate for the needs of the particular situation, and that the decisions can be made by computer software programs. Classes can be created containing almost any number of scaffolds, and the criteria selected can be based on increasingly exacting criteria until an arbitrary number of scaffolds is arrived at for each class that is deemed to be advantageous.

Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference. Vely F. et al., BIAcore® analysis to test phosphopeptide-SH2 domain interactions, Methods in Molecular Biology. 121:313-21, 2000; Liparoto et al., Biosensor analysis of the interleukin-2 receptor complex, Journal of Molecular Recognition. 12:316-21, 1999; Lipschultz et al., Experimental design for analysis of complex kinetics using surface plasmon resonance, Methods. 20(3):310-8, 2000; Malmqvist., BIACORE: an affinity biosensor system for characterization of biomolecular interactions, Biochemical Society Transactions 27:335-40, 1999; Alfthan, Surface plasmon resonance biosensors as a tool in antibody engineering, Biosensors & Bioelectronics. 13:653-63, 1998; Fivash et al., BIAcore for macromolecular interaction, Current Opinion in Biotechnology. 9:97-101, 1998; Price et al.; Summary report on the ISOBM TD-4 Workshop: analysis of 56 monoclonal antibodies against the MUC1 mucin. Tumour Biology 19 Suppl 1:1-20, 1998; Malmqvist et al, Biomolecular interaction analysis: affinity biosensor technologies for functional analysis of proteins, Current Opinion in Chemical Biology. 1:378-83, 1997; O'Shannessy et al., Interpretation of deviations from pseudo-first-order kinetic behavior in the characterization of ligand binding by biosensor technology, Analytical Biochemistry. 236:275-83, 1996; Malmborg et al., BIAcore as a tool in antibody engineering, Journal of Immunological Methods. 183:7-13, 1995; Van Regenmortel, Use of biosensors to characterize recombinant proteins, Developments in Biological Standardization. 83:143-51, 1994; and O'Shannessy, Determination of kinetic rate and equilibrium binding constants for macromolecular interactions: a critique of the surface plasmon resonance literature, Current Opinions in Biotechnology. 5:65-71, 1994.

BIAcore® uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator. Similarly, when ligands to a target are sought, known ligands of the target can be present in control/calibration assay wells.

Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of calorimetric assays for the detection of peroxides, as disclosed in Example 1(b) and Gordon, A. J. and Ford, R. A., The Chemist's Companion: A Handbook Of Practical Data, Techniques, And References, John Wiley and Sons, N.Y., 1972, Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., Spectrophotometry and Spectrofluorometry: A Practical Approach, pp. 91-114, IRL Press Ltd. (1987); and Bell, Spectroscopy In Biochemistry, Vol. I, pp. 155-194, CRC Press (1981).

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, Oreg.). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., Application of Fluorescence Polarization Assays in High-Throughput Screening, Genetic Engineering News, 17:27, 1997.

FP is particularly desirable since its readout is independent of the emission intensity (Checovich, W. J., et al., Nature 375:254-256, 1995; Dandliker, W. B., et al., Methods in Enzymology 74:3-28, 1981) and is thus insensitive to the presence of colored compounds that quench fluorescence emission. FP and FRET (see below) are well-suited for identifying compounds that block interactions between sphingolipid receptors and their ligands. See, for example, Parker et al., Development of high throughput screening assays using fluorescence polarization: nuclear receptor-ligand-binding and kinase/phosphatase assays, J Biomol Screen 5:77-88, 2000.

Fluorophores, derived from sphingolipids that may be used in FP assays are commercially available. For example, Molecular Probes (Eugene, Oreg.) currently sells sphingomyelin and one ceramide flurophores. These are, respectively, N-(4,4-difluoro-5,7-dimethyl-4-bora-3a,4a-diaza-s-indacene-3-pentanoyl)sphingosyl phosphocholine (BODIPY® FL C5-sphingomyelin); N-(4,4-difluoro-5,7-dimethyl-4-bora-3a,4a-diaza-s-indacene-3-dodecanoyl)sphingosyl phosphocholine (BODIPY®) FL C12-sphingomyelin); and N-(4,4-difluoro-5,7-dimethyl-4-bora-3a,4a-diaza-s-indacene-3-pentanoyl)sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION® fluorescence polarization system (Tecan ΔG, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., Curr. Biol. 6:178-182, 1996; Mitra et al., Gene 173:13-17 1996; and Selvin et al., Meth. Enzymol. 246:300-345, 1995. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., J. Lipid Res. 38:2365-2373 (1997); Kahl et al., Anal. Biochem. 243:282-283 (1996); Undenfriend et al., Anal. Biochem. 161:494-500 (1987)). See also U.S. Pat. Nos. 4,626,513 and 4,568,649, and European Patent No. 0,154,734. One commercially available system uses FLASHPLATE® scintillant-coated plates (NEN Life Science Products, Boston, Mass.).

The target molecule can be bound to the scintillator plates by a variety of well known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT® microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., Anal. Biochem. 257:112-119, 1998).

Assay Compounds and Molecular Scaffolds

As described above, preferred characteristics of a scaffold include being of low molecular weight (e.g., less than 350 Da, or from about 100 to about 350 daltons, or from about 150 to about 300 daltons). Preferably clog P of a scaffold is from −1 to 8, more preferably less than 6, 5, or 4, most preferably less than 3. In particular embodiments the clogP is in a range −1 to an upper limit of 2, 3, 4, 5, 6, or 8; or is in a range of 0 to an upper limit of 2, 3, 4, 5, 6, or 8. Preferably the number of rotatable bonds is less than 5, more preferably less than 4. Preferably the number of hydrogen bond donors and acceptors is below 6, more preferably below 5. An additional criterion that can be useful is a Polar Surface Area of less than 100. Guidance that can be useful in identifying criteria for a particular application can be found in Lipinski et al., Advanced Drug Delivery Reviews 23 (1997) 3-25, which is hereby incorporated by reference in its entirety.

A scaffold will preferably bind to a given protein binding site in a configuration that causes substituent moieties of the scaffold to be situated in pockets of the protein binding site. Also, possessing chemically tractable groups that can be chemically modified, particularly through synthetic reactions, to easily create a combinatorial library can be a preferred characteristic of the scaffold. Also preferred can be having positions on the scaffold to which other moieties can be attached, which do not interfere with binding of the scaffold to the protein(s) of interest but do cause the scaffold to achieve a desirable property, for example, active transport of the scaffold to cells and/or organs, enabling the scaffold to be attached to a chromatographic column to facilitate analysis, or another desirable property. A molecular scaffold can bind to a target molecule with any affinity, such as binding with an affinity measurable as about three times the standard deviation of the background signal, or at high affinity, moderate affinity, low affinity, very low affinity, or extremely low affinity.

Thus, the above criteria can be utilized to select many compounds for testing that have the desired attributes. Many compounds having the criteria described are available in the commercial market, and may be selected for assaying depending on the specific needs to which the methods are to be applied. In some cases sufficiently large numbers of compounds may meet specific criteria that additional methods to group similar compounds may be helpful. A variety of methods to assess molecular similarity, such as the Tanimoto coefficient have been used, see Willett et al, Journal of Chemical Information and Computer Science 38 (1998), 983-996. These can be used to select a smaller subset of a group of highly structurally redundant compounds. In addition, cluster analysis based on relationships between the compounds, or structural components of the compound, can also be carried out to the same end; see Lance and Williams Computer Journal 9 (1967) 373-380, Jarvis and Patrick IEEE Transactions in Computers C-22 (1973) 1025-1034 for clustering algorithms, and Downs et al. Journal of Chemical Information and Computer Sciences-34 (1994) 1094-1102 for a review of these methods applied to chemical problems. One method of deriving the chemical components of a large group of potential scaffolds is to virtually break up the compound at rotatable bonds so as to yield components of no less than 10 atoms. The resulting components may be clustered based on some measure of similarity, e.g. the Tanimoto coefficient, to yield the common component groups in the original collection of compounds. For each component group, all compounds containing that component may be clustered, and the resulting clusters used to select a diverse set of compounds containing a common chemical core structure. In this fashion, a useful library of scaffolds may be derived even from millions of commercial compounds.

A “compound library” or “library” is a collection of different compounds having different chemical structures. A compound library is screenable, that is, the compound library members therein may be subject to screening assays. In preferred embodiments, the library members can have a molecular weight of from about 100 to about 350 daltons, or from about 150 to about 350 daltons.

Libraries of the present invention can contain at least one compound that binds to the target molecule at low affinity. Libraries of candidate compounds can be assayed by many different assays, such as those described above, e.g., a fluorescence polarization assay. Libraries may consist of chemically synthesized peptides, peptidomimetics, or arrays of combinatorial chemicals that are large or small, focused or nonfocused. By “focused” it is meant that the collection of compounds is prepared using the structure of previously characterized compounds and/or pharmacophores.

Compound libraries may contain molecules isolated from natural sources, artificially synthesized molecules, or molecules synthesized, isolated, or otherwise prepared in such a manner so as to have one or more moieties variable, e.g., moieties that are independently isolated or randomly synthesized. Types of molecules in compound libraries include but are not limited to organic compounds, polypeptides and nucleic acids as those terms are used herein, and derivatives, conjugates and mixtures thereof.

Compound libraries useful for the invention may be purchased on the commercial market or prepared or obtained by any means including, but not limited to, combinatorial chemistry techniques, fermentation methods, plant and cellular extraction procedures and the like (see, e.g., Cwirla et al., Biochemistry 1990, 87, 6378-6382; Houghten et al., Nature 1991, 354, 84-86; Lam et al., Nature 1991, 354, 82-84; Brenner et al., Proc. Natl. Acad. Sci. USA 1992, 89, 5381-5383; R. A. Houghten, Trends Genet. 1993, 9, 235-239; E. R. Felder, Chimia 1994, 48, 512-541; Gallop et al., J. Med. Chem. 1994, 37, 1233-¹²⁵I; Gordon et al., J. Med. Chem. 1994, 37, 1385-1401; Carell et al., Chem. Biol. 1995, 3, 171-183; Madden et al., Perspectives in Drug Discovery and Design 2, 269-282; Lebl et al., Biopolymers 1995, 37 177-198); small molecules assembled around a shared molecular structure; collections of chemicals that have been assembled by various commercial and noncommercial groups, natural products; extracts of marine organisms, fungi, bacteria, and plants.

Preferred libraries can be prepared in a homogenous reaction mixture, and separation of unreacted reagents from members of the library is not required prior to screening. Although many combinatorial chemistry approaches are based on solid state chemistry, liquid phase combinatorial chemistry is capable of generating libraries (Sun CM., Recent advances in liquid-phase combinatorial chemistry, Combinatorial Chemistry & High Throughput Screening. 2:299-318, 1999).

Libraries of a variety of types of molecules are prepared in order to obtain members therefrom having one or more preselected attributes that can be prepared by a variety of techniques, including but not limited to parallel array synthesis (Houghton, Annu Rev Pharmacol Toxicol 2000 40:273-82, Parallel array and mixture-based synthetic combinatorial chemistry; solution-phase combinatorial chemistry (Merritt, Comb Chem High Throughput Screen 1998 1(2):57-72, Solution phase combinatorial chemistry, Coe et al., Mol Divers 1998-99;4(1):31-8, Solution-phase combinatorial chemistry, Sun, Comb Chem High Throughput Screen 1999 2(6):299-318, Recent advances in liquid-phase combinatorial chemistry); synthesis on soluble polymer (Gravert et al., Curr Opin Chem Biol 1997 1(1):107-13, Synthesis on soluble polymers: new reactions and the construction of small molecules); and the like. See, e.g., Dolle et al., J Comb Chem 1999 1(4):235-82, Comprehensive survey of cominatorial library synthesis: 1998. Freidinger R M., Nonpeptidic ligands for peptide and protein receptors, Current Opinion in Chemical Biology; and Kundu et al., Prog Drug Res 1999;53:89-156, Combinatorial chemistry: polymer supported synthesis of peptide and non-peptide libraries). Compounds may be clinically tagged for ease of identification (Chabala, Curr Opin Biotechnol 1995 6(6):633-9, Solid-phase combinatorial chemistry and novel tagging methods for identifying leads).

The combinatorial synthesis of carbohydrates and libraries containing oligosaccharides have been described (Schweizer et al., Curr Opin Chem Biol 1999 3(3):291-8, Combinatorial synthesis of carbohydrates). The synthesis of natural-product based compound libraries has been described (Wessjohann, Curr Opin Chem Biol 2000 4(3):303-9, Synthesis of natural-product based compound libraries).

Libraries of nucleic acids are prepared by various techniques, including byway of non-limiting example the ones described herein, for the isolation of aptamers. Libraries that include oligonucleotides and polyaminooligonucleotides (Markiewicz et al., Synthetic oligonucleotide combinatorial libraries and their applications, Farmaco. 55:174-7, 2000) displayed on streptavidin magnetic beads are known. Nucleic acid libraries are known that can be coupled to parallel sampling and be deconvoluted without complex procedures such as automated mass spectrometry (Enjalbal C. Martinez J. Aubagnac J L, Mass spectrometry in combinatorial chemistry, Mass Spectrometry Reviews. 19:139-61, 2000) and parallel tagging. (Perrin D M., Nucleic acids for recognition and catalysis: landmarks, limitations, and looking to the future, Combinatorial Chemistry & High Throughput Screening 3:243-69).

Peptidomimetics are identified using combinatorial chemistry and solid phase synthesis (Kim H O. Kahn M., A merger of rational drug design and combinatorial chemistry: development and application of peptide secondary structure mimetics, Combinatorial Chemistry & High Throughput Screening 3:167-83, 2000; al-Obeidi, Mol Biotechnol 1998 9(3):205-23, Peptide and peptidomimetric libraries. Molecular diversity and drug design). The synthesis may be entirely random or based in part on a known polypeptide.

Polypeptide libraries can be prepared according to various techniques. In brief, phage display techniques can be used to produce polypeptide ligands (Gram H., Phage display in proteolysis and signal transduction, Combinatorial Chemistry & High Throughput Screening. 2:19-28, 1999) that may be used as the basis for synthesis of peptidomimetics. Polypeptides, constrained peptides, proteins, protein domains, antibodies, single chain antibody fragments, antibody fragments, and antibody combining regions are displayed on filamentous phage for selection.

Large libraries of individual variants of human single chain Fv antibodies have been produced. See, e.g., Siegel R W. Allen B. Pavlik P. Marks J D. Bradbury A., Mass spectral analysis of a protein complex using single-chain antibodies selected on a peptide target: applications to functional genomics, Journal of Molecular Biology 302:285-93, 2000; Poul M A. Becerril B. Nielsen U B. Morisson P. Marks J D., Selection of tumor-specific internalizing human antibodies from phage libraries. Source Journal of Molecular Biology. 301:1149-61, 2000; Amersdorfer P. Marks J D., Phage libraries for generation of anti-botulinum scFv antibodies, Methods in Molecular Biology. 145:219-40, 2001; Hughes-Jones N C. Bye J M. Gorick B D. Marks J D. Ouwehand W H., Synthesis of Rh Fv phage-antibodies using VH and VL germline genes, British Journal of Haematology. 105:811-6, 1999; McCall A M. Amoroso A R. Sautes C. Marks J D. Weiner L M., Characterization of anti-mouse Fe gamma RII single-chain Fv fragments derived from human phage display libraries, Immunotechnology. 4:71-87, 1998; Sheets M D. Amersdorfer P. Finnern R. Sargent P. Lindquist E. Schier R. Hemingsen G. Wong C. Gerhart J C. Marks J D. Lindquist E., Efficient construction of a large nonimmune phage antibody library: the production of high-affinity human single-chain antibodies to protein antigens (published erratum appears in Proc Natl Acad Sci USA 1999 96:795), Proc Natl AcadSci USA 95:6157-62, 1998).

Focused or smart chemical and pharmacophore libraries can be designed with the help of sophisticated strategies involving computational chemistry (e.g., Kundu B. Khare S K. Rastogi S K., Combinatorial chemistry: polymer supported synthesis of peptide and non-peptide libraries, Progress in Drug Research 53:89-156, 1999) and the use of structure-based ligands using database searching and docking, de novo drug design and estimation of ligand binding affinities (Joseph-McCarthy D., Computational approaches to structure-based ligand design, Pharmacology & Therapeutics 84:179-91, 1999; Kirkpatrick D L. Watson S. Ulhaq S., Structure-based drug design: combinatorial chemistry and molecular modeling, Combinatorial Chemistry & High Throughput Screening. 2:211-21, 1999; Eliseev A V. Lehn J M., Dynamic combinatorial chemistry: evolutionary formation and screening of molecular libraries, Current Topics in Microbiology & Immunology 243:159-72, 1999; Bolger et al., Methods Enz. 203:21-45, 1991; Martin, Methods Enz. 203:587-613, 1991; Neidle et al., Methods Enz. 203:433-458, 1991; U.S. Pat. No. 6,178,384).

Selecting a library of potential scaffolds and a set of assays measuring binding to representative target molecules which are in a particular protein family thus allows the creation of a data set profiling binding of the library to the target protein family. Groups of scaffolds with different sets of binding properties can be identified using the information within this dataset. Thus, groups of scaffolds binding to one, two or three members of the family may be selected for particular applications.

In many cases, a group of scaffolds exhibiting binding to two or more members of a target protein family will contain scaffolds with a greater likelihood that such binding results from specific interactions with the individual target proteins. This would be expected to substantially reduce the effect of so-called “promiscuous inhibitors” which severely complicate the interpretation of screening assays (see McGovern et al Journal of Medicinal Chemistry 45:1712-22, 2002). Thus, in many preferred applications the property of displaying binding to multiple target molecules in a protein family may be used as a selection criteria to identify molecules with desirable properties. In addition, groups of scaffolds binding to specific subsets of a set of potential target molecules may be selected. Such a case would include the subset of scaffolds that bind to any two of three or three of five members of a target protein family.

Such subsets may also be used in combination or opposition to further define a group of scaffolds that have additional desirable properties. This would be of significant utility in cases where inhibiting some members of a protein family had known desirable effects, such as inhibiting tumor growth, whereas inhibiting other members of the protein family which were found to be essential for normal cell function would have undesirable effects. A criteria that would be useful in such a case includes selecting the subset of scaffolds binding to any two of three desirable target molecules and eliminating from this group any that bound to more than one of any three undesirable target molecules.

Crystallography

After binding compounds have been determined, the orientation of compound bound to target is determined. Preferably this determination involves crystallography on co-crystals of molecular scaffold compounds with target. Most protein crystallographic platforms can preferably be designed to analyze up to about 500 co-complexes of compounds, ligands, or molecular scaffolds bound to protein targets due to the physical parameters of the instruments and convenience of operation.

If the number of scaffolds that have binding activity exceeds a number convenient for the application of crystallography methods, the scaffolds can be placed into groups based on having at least one common chemical structure or other desirable characteristics, and representative compounds can be selected from one or more of the classes. Classes can be made with increasingly exacting criteria until a desired number of classes (e.g., 10, 20, 50, 100, 200, 300, 400, 500) is obtained. The classes can be based on chemical structure similarities between molecular scaffolds in the class, e.g., all possess a pyrrole ring, benzene ring, or other chemical feature. Likewise, classes can be based on shape characteristics, e.g., space-filling characteristics.

The co-crystallography analysis can be performed by co-complexing each scaffold with its target, e.g., at concentrations of the scaffold that showed activity in the screening assay. This co-complexing can, for example, be accomplished with the use of low percentage organic solvents with the target molecule and then concentrating the target with each of the scaffolds. In preferred embodiments these solvents are less than 5% organic solvent such as dimethyl sulfoxide (DMSO), ethanol, methanol, or ethylene glycol in water or another aqueous solvent.

Each scaffold complexed to the target molecule can then be screened with a suitable number of crystallization screening conditions at appropriate temperature, e.g., both 4 and 20 degrees. In preferred embodiments, about 96 crystallization screening conditions can be performed in order to obtain sufficient information about the co-complexation and crystallization conditions, and the orientation of the scaffold at the binding site of the target molecule. Crystal structures can then be analyzed to determine how the bound scaffold is oriented physically within the binding site or within one or more binding pockets of the molecular family member.

It is desirable to determine the atomic coordinates of the compounds bound to the target proteins in order to determine which is a most suitable scaffold for the protein family. X-ray crystallographic analysis is therefore most preferable for determining the atomic coordinates. Those compounds selected can be further tested with the application of medicinal chemistry. Compounds can be selected for medicinal chemistry testing based on their binding position in the target molecule. For example, when the compound binds at a binding site, the compound's binding position in the binding site of the target molecule can be considered with respect to the chemistry that can be performed on chemically tractable structures or sub-structures of the compound, and how such modifications on the compound are expected to interact with structures or sub-structures on the binding site of the target. Thus, one can explore the binding site of the target and the chemistry of the scaffold in order to make decisions on how to modify the scaffold to arrive at a ligand with higher potency and/or selectivity.

The structure of the target molecule bound to the compound may also be superimposed or aligned with other structures of members of the same protein family. In this way modifications of the scaffold can be made to enhance the binding to members of the target family in general, thus enhancing the utility of the scaffold library. Different useful alignments may be generated, using a variety of criteria such as minimal RMSD superposition of α-carbons or backbone atoms of homologous or structurally related regions of the proteins.

These processes allow for more direct design of ligands, by utilizing structural and chemical information obtained directly from the co-complex, thereby enabling one to more efficiently and quickly design lead compounds that are likely to lead to beneficial drug products. In various embodiments it may be desirable to perform co-crystallography on all scaffolds that bind, or only those that bind with a particular affinity, for example, only those that bind with high affinity, moderate affinity, low affinity, very low affinity, or extremely low affinity. It may also be advantageous to perform co-crystallography on a selection of scaffolds that bind with any combination of affinities.

Standard X-ray protein diffraction studies such as by using a Rigaku RU-200@ (Rigaku, Tokyo, Japan) with an X-ray imaging plate detector or a synchrotron beam-line can be performed on co-crystals and the diffraction data measured on a standard X-ray detector, such as a CCD detector or an X-ray imaging plate detector.

Performing X-ray crystallography on about 200 co-crystals should generally lead to about 50 co-crystal structures, which should provide about 10 scaffolds for validation in chemistry, which should finally result in about 5 selective leads for target molecules.

Additives that promote co-crystallization can of course be included in the target molecule formulation in order to enhance the formation of co-crystals. In the case of proteins or enzymes, the scaffold to be tested can be added to the protein formulation, which is preferably present at a concentration of approximately 1 mg/ml. The formulation can also contain between 0%-10% (v/v) organic solvent, e.g. DMSO, methanol, ethanol, propane diol, or 1,3 dimethyl propane diol (MPD) or some combination of those organic solvents. Compounds are preferably solubilized in the organic solvent at a concentration of about 10 mM and added to the protein sample at a concentration of about 100 mM. The protein-compound complex is then concentrated to a final concentration of protein of from about 5 to about 20 mg/ml. The complexation and concentration steps can conveniently be performed using a 96 well formatted concentration apparatus (e.g., Amicon Inc., Piscataway, N.J.). Buffers and other reagents present in the formulation being crystallized can contain other components that promote crystallization or are compatible with crystallization conditions, such as DTT, propane diol, glycerol.

The crystallization experiment can be set-up by placing small aliquots of the concentrated protein-compound complex (e.g., 1 μl) in a 96 well format and sampling under 96 crystallization conditions. (Other formats can also be used, for example, plates with fewer or more wells.) Crystals can typically be obtained using standard crystallization protocols that can involve the 96 well crystallization plate being placed at different temperatures. Co-crystallization varying factors other than temperature can also be considered for each protein-compound complex if desirable. For example, atmospheric pressure, the presence or absence of light or oxygen, a change in gravity, and many other variables can all be tested. The person of ordinary skill in the art will realize other variables that can advantageously be varied and considered. Conveniently, commercially available crystal screening plates with specified conditions in individual wells can be utilized.

Virtual Assays

Commercially available software that generates three-dimensional graphical representations of the complexed target and compound from a set of coordinates provided can be used to illustrate and study how a compound is oriented when bound to a target. (e.g., InsightII®, Accelerys, San Diego, Calif.; or Sybyl®, Tripos Associates, St. Louis, Mo.). Thus, the existence of binding pockets at the binding site of the targets can be particularly useful in the present invention. These binding pockets are revealed by the crystallographic structure determination and show the precise chemical interactions involved in binding the compound to the binding site of the target. The person of ordinary skill will realize that the illustrations can also be used to decide where chemical groups might be added, substituted, modified, or deleted from the scaffold to enhance binding or another desirable effect, by considering where unoccupied space is located in the complex and which chemical substructures might have suitable size and/or charge characteristics to fill it. The person of ordinary skill will also realize that regions within the binding site can be flexible and its properties can change as a result of scaffold binding, and that chemical groups can be specifically targeted to those regions to achieve a desired effect. Specific locations on the molecular scaffold can be considered with reference to where a suitable chemical substructure can be attached and in which conformation, and which site has the most advantageous chemistry available.

An understanding of the forces that bind the compounds to the target proteins reveals which compounds can most advantageously be used as scaffolds, and which properties can most effectively be manipulated in the design of ligands. The person of ordinary skill will realize that steric, ionic, polar, hydrogen bond, and other forces can be considered for their contribution to the maintenance or enhancement of the target-compound complex. Additional data can be obtained with automated computational methods, such as docking and/or molecular dynamics simulations, which can afford a measure of the energy of binding. In addition, to account for other effects such as entropies of binding and desolvation penalties, methods which provide a measure of these effects can be integrated into the automated computational approach. The compounds selected can be used to generate information about the chemical interactions with the target or for elucidating chemical modifications that can enhance selectivity of binding of the compound.

An exemplary calculation of binding energies between protein-ligand complexes can be obtained using the FlexX score (an implementation of the Bohm scoring function) within the Tripos software suite (Tripos Associates, St. Louis, Mo.). The form for that equation is shown below:

ΔGbind=ΔGtr+ΔGhb+ΔGion+ΔGlipo+ΔGarom+ΔGrot

where: ΔGtr is a constant term that accounts for the overall loss of rotational and translational entropy of the lignand, ΔGhb accounts for hydrogen bonds formed between the ligand and protein, ΔGion accounts for the ionic interactions between the ligand and protein, ΔGlipo accounts for the lipophilic interaction that corresponds to the protein-ligand contact surface, ΔGarom accounts for interactions between aromatic rings in the protein and ligand, and ΔGrot accounts for the entropic penalty of restricting rotatable bonds in the ligand upon binding. The calculated binding energy for compounds that bind strongly to a given target will likely be lower than −25 kcal/mol, while the calculated binding affinity for a good scaffold or an unoptimized compound will generally be in the range of −15 to −20. The penalty for restricting a linker such as the ethylene glycol or hexatriene is estimated as typically being in the range of +5 to +15.

This method estimates the free energy of binding that a lead compound should have to a target protein for which there is a crystal structure, and it accounts for the entropic penalty of flexible linkers. It can therefore be used to estimate the penalty incurred by attaching linkers to molecules being screened and the binding energy that a lead compound must attain in order to overcome the penalty of the linker. The method does not account for solvation, and the entropic penalty is likely overestimated when the linkers are bound to the solid phase through an additional binding complex, e.g., a biotin:streptavidin complex.

Another exemplary method for calculating binding energies is the MM-PBSA technique (Massova and Kollman, Journal of the American Chemical Society 121:8133-43,1999; Chong et al, Proceedings of the National Academy of Sciences 96:14330-5,1999; Donini and Kollman, Journal of Medicinal Chemistry 43:4180-8,2000). This method uses a Molecular Dynamics approach to generate many sample configurations of the compound and complexed target molecule, then calculates an interaction energy using the well-known AMBER force field (Cornell, et al Journal of the American Chemical Society 117:5179-97 1995) with corrections for desolvation and entropy of binding from the ensemble.

Use of this method yields binding energies highly correlated with those found experimentally. The absolute binding energies calculated with this method are reasonably accurate, and the variation of binding energies is approximately linear with a slope of 1+/−0.5. Thus, the binding energies of compounds interacting strongly with a given target will be lower than about −8 kcal/mol, while a binding energy of a good scaffold or unoptimized compound will be in the range of −3 to −7 kcal/mol.

Computer models, such as homology models (i.e., based on a known, experimentally derived structure) can be constructed using data from the co-crystal structures. A computer program such as Modeller (Accelrys, San Diego Calif.) may be used to assign the three dimensional coordinates to a protein sequence using an alignment of sequences and a set or sets of template coordinates. When the target molecule is a protein or enzyme, preferred co-crystal structures for making homology models contain high sequence identity in the binding site of the protein sequence being modeled, and the proteins will preferentially also be within the same class and/or fold family. Knowledge of conserved residues in active sites of a protein class can be used to select homology models that accurately represent the binding site. Homology models can also be used to map structural information from a surrogate protein where an apo or co-crystal structure exists to the target protein.

Virtual screening methods, such as docking, can also be used to predict the binding configuration and affinity of scaffolds, compounds, and/or combinatorial library members to homology models. Using this data, and carrying out “virtual experiments” using computer software can save substantial resources and allow the person of ordinary skill to make decisions about which compounds can be suitable scaffolds or ligands, without having to actually synthesize the ligand and perform co-crystallization. Decisions thus can be made about which compounds merit actual synthesis and co-crystallization. An understanding of such chemical interactions aids in the discovery and design of drugs that interact more advantageously with target proteins and/or are more selective for one protein family member over others. Thus, applying these principles, compounds with superior properties can be discovered.

Ligand Design and Preparation

The design and preparation of ligands can be performed with or without structural and/or co-crystallization data by considering the chemical structures in common between the active scaffolds of a set. In this process structure-activity hypotheses can be formed and those chemical structures found to be present in a substantial number of the scaffolds, including those that bind with low affinity, can be presumed to have some effect on the binding of the scaffold. This binding can be presumed to induce a desired biochemical effect when it occurs in a biological system (e.g., a treated mammal). New or modified scaffolds or combinatorial libraries derived from scaffolds can be tested to disprove the maximum number of binding and/or structure-activity hypotheses. The remaining hypotheses can then be used to design ligands that achieve a desired binding and biochemical effect.

But in many cases it will be preferred to have co-crystallography data for consideration of how to modify the scaffold to achieve the desired binding effect (e.g., binding at higher affinity or with higher selectivity). Using the case of proteins and enzymes, co-crystallography data shows the binding pocket of the protein with the molecular scaffold bound to the binding site, and it will be apparent that a modification can be made to a chemically tractable group on the scaffold. For example, a small volume of space at a protein binding site or pocket might be filled by modifying the scaffold to include a small chemical group that fills the volume. Filling the void volume can be expected to result in a greater binding affinity, or the loss of undesirable binding to another member of the protein family. Similarly, the co-crystallography data may show that deletion of a chemical group on the scaffold may decrease a hindrance to binding and result in greater binding affinity or specificity.

Various software packages have implemented techniques which facilitate the identification and characterization of interactions of potential binding sites from complex structure, or from an apo structure of a target molecule, i.e. one without a compound bound (e.g. SiteID, Tripos Associates, St. Louis Mo. and SiteFinder, Chemical Computing Group, Montreal Canada, GRID, Molecular Discovery Ltd., London UK). Such techniques can be used with the coordinates of a complex between the scaffold of interest and a target molecule, or these data in conjunction with data for a suitably aligned or superimposed related target molecule, in order to evaluate changes to the scaffold that would enhance binding to the desired target molecule structure or structures. Molecular Interaction Field-computing techniques, such as those implemented in the program GRID, result in energy data for particular positive and negative binding interactions of different computational chemical probes being mapped to the vertices of a matrix in the coordinate space of the target molecule. These data can then be analyzed for areas of substitution around the scaffold binding site which are predicted to have a favorable interaction for a particular target molecule. Compatible chemical substitution on the scaffold e.g. a methyl, ethyl or phenyl group in a favorable interaction region computed from a hydrophobic probe, would be expected to result in an improvement in affinity of the scaffold. Conversely, a scaffold could be made more selective for a particular target molecule by making such a substitution in a region predicted to have an unfavorable hydrophobic interaction in a second, related undesirable target molecule.

It can be desirable to take advantage of the presence of a charged chemical group located at the binding site or pocket of the protein. For example, a positively charged group can be complemented with a negatively charged group introduced on the molecular scaffold. This can be expected to increase binding affinity or binding specificity, thereby resulting in a more desirable ligand. In many cases, regions of protein binding sites or pockets are known to vary from one family member to another based on the amino acid differences in those regions. Chemical additions in such regions can result in the creation or elimination of certain interactions (e.g., hydrophobic, electrostatic, or entropic) that allow a compound to be more specific for one protein target over another or to bind with greater affinity, thereby enabling one to synthesize a compound with greater selectivity or affinity for a particular family member. Additionally, certain regions can contain amino acids that are known to be more flexible than others. This often occurs in amino acids contained in loops connecting elements of the secondary structure of the protein, such as alpha helices or beta strands. Additions of chemical moieties can also be directed to these flexible regions in order to increase the likelihood of a specific interaction occurring between the protein target of interest and the compound. Virtual screening methods can also be conducted in silico to assess the effect of chemical additions, subtractions, modifications, and/or substitutions on compounds with respect to members of a protein family or class.

The addition, subtraction, or modification of a chemical structure or sub-structure to a scaffold can be performed with any suitable chemical moiety. For example the following moieties, which are provided by way of example and are not intended to be limiting, can be utilized: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (e.g., forming a ketone or N-oxide) or a sulphur atom (e.g., forming a thiol, thione, di-alkylsulfoxide or sulfone) are all examples of moieties that can be utilized.

Additional examples of structures or sub-structures that may be utilized are an aryl optionally substituted with one, two, or three substituents independently selected from the group consisting of alkyl, alkoxy, halogen, trihalomethyl, carboxylate, nitro, and ester moieties; an amine of formula —NX₂X₃, where X₂ and X₃ are independently selected from the group consisting of hydrogen, saturated or unsaturated alkyl, and homocyclic or heterocyclic ring moieties; halogen or trihalomethyl; a ketone of formula —COX₄, where X₄ is selected from the group consisting of alkyl and homocyclic or heterocyclic ring moieties; a carboxylic acid of formula —(X₅)ₙCOOH or ester of formula (X₆)ₙCOOX₇, where X₅, X₆, and X₇ and are independently selected from the group consisting of alkyl and homocyclic or heterocyclic ring moieties and where n is 0 or 1; an alcohol of formula (X₈)ₙOH or an alkoxy moiety of formula —(X₈)ₙOX₉, where X₈ and X₉ are independently selected from the group consisting of saturated or unsaturated alkyl and homocyclic or heterocyclic ring moieties, wherein said ring is optionally substituted with one or more substituents independently selected from the group consisting of alkyl, alkoxy, halogen, trihalomethyl, carboxylate, nitro, and ester and where n is 0 or 1; an amide of formula NHCOX₁₀, where X₁₀ is selected from the group consisting of alkyl, hydroxyl, and homocyclic or heterocyclic ring moieties, wherein said ring is optionally substituted with one or more substituents independently selected from the group consisting of alkyl, alkoxy, halogen, trihalomethyl, carboxylate, nitro, and ester; SO₂, NX₁₁ X₁₂, where X₁₁ and X₁₂ are selected from the group consisting of hydrogen, alkyl, and homocyclic or heterocyclic ring moieties; a homocyclic or heterocyclic ring moiety optionally substituted with one, two, or three substituents independently selected from the group consisting of alkyl, alkoxy, halogen, trihalomethyl, carboxylate, nitro, and ester moieties; an aldehyde of formula —COH; a sulfone of formula —SO₂X₁₃, where X₁₃ is selected from the group consisting of saturated or unsaturated alkyl and homocyclic or heterocyclic ring moieties; and a nitro of formula —NO₂.

With respect to the compounds and functional groups, the following definitions apply.

“Halo” or “Halogen”—alone or in combination means all halogens, that is, chloro (Cl), fluoro (F), bromo (Br), iodo (I).

“Hydroxyl” refers to the group —OH.

“Thiol” or “mercapto” refers to the group —SH.

“Alkyl”—alone or in combination means an alkane-derived radical containing from 1 to 20, preferably 1 to 15, carbon atoms (unless specifically defined). It is a straight chain alkyl, branched alkyl or cycloalkyl. Preferably, straight or branched alkyl groups containing from 1-15, more preferably 1 to 8, even more preferably 1-6, yet more preferably 1-4 and most preferably 1-2, carbon atoms, such as methyl, ethyl, propyl, isopropyl, butyl, t-butyl and the like. The term “lower alkyl” is used herein to describe the straight chain alkyl groups described immediately above. Preferably, cycloalkyl groups are monocyclic, bicyclic or tricyclic ring systems of 3-8, more preferably 3-6, ring members per ring, such as cyclopropyl, cyclopentyl, cyclohexyl, adamantyl and the like. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl. A substituted alkyl is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

The term “saturated alkyl” refers to an alkyl moiety that does not contain any alkene or alkyne moieties. The alkyl moiety can be branched or non-branched.

The term “unsaturated alkyl” refers to an alkyl moiety that contains at least one alkene or alkyne moiety. The alkyl moiety can be branched or non-branched.

“Alkenyl”—alone or in combination means a straight, branched, or cyclic hydrocarbon containing 2-20, preferably 2-17, more preferably 2-10, even more preferably 2-8, most preferably 2-4, carbon atoms and at least one, preferably 1-3, more preferably 1-2, most preferably one, carbon to carbon double bond. In the case of a cycloalkyl group, conjugation of more than one carbon to carbon double bond is not such as to confer aromaticity to the ring. Carbon to carbon double bonds may be either contained within a cycloalkyl portion, with the exception of cyclopropyl, or within a straight chain or branched portion. Examples of alkenyl groups include ethenyl, propenyl, isopropenyl, butenyl, cyclohexenyl, cyclohexenylalkyl and the like. A substituted alkenyl is the straight chain alkenyl, branched alkenyl or cycloalkenyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, carboxy, alkoxycarbonyl, aryloxycarbonyl, heteroaryloxycarbonyl, or the like attached at any available point to produce a stable compound.

“Alkynyl”—alone or in combination means a straight or branched hydrocarbon containing 2-20, preferably 2-17, more preferably 2-10, even more preferably 2-8, most preferably 2-4, carbon atoms containing at least one, preferably one, carbon to carbon triple bond. Examples of alkynyl groups include ethynyl, propynyl, butynyl and the like. A substituted alkynyl refers to the straight chain alkynyl or branched alkenyl defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like attached at any available point to produce a stable compound.

“Alkyl alkenyl” refers to a group —R—CR′═CR′″R″″, where R is lower alkyl, or substituted lower alkyl, R′, R′″, R″″ may independently be hydrogen, halogen, lower alkyl, substituted lower alkyl, acyl, aryl, substituted aryl, hetaryl, or substituted hetaryl as defined below.

“Alkyl alkynyl” refers to a groups —RCCR′ where R is lower alkyl or substituted lower alkyl, R′ is hydrogen, lower alkyl, substituted lower alkyl, acyl, aryl, substituted aryl, hetaryl, or substituted hetaryl as defined below.

“Alkoxy” denotes the group —OR, where R is lower alkyl, substituted lower alkyl, acyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

“Alkylthio” or “thioalkoxy” denotes the group —SR, —S(O)ₙ₌₁₋₂—R, where R is lower alkyl, substituted lower alkyl, aryl, substituted aryl, aralkyl or substituted aralkyl as defined herein.

“Acyl” denotes groups —C(O)R, where R is hydrogen, lower alkyl substituted lower alkyl, aryl, substituted aryl and the like as defined herein.

“Aryloxy” denotes groups —OAr, where Ar is an aryl, substituted aryl, heteroaryl, or substituted heteroaryl group as defined herein.

“Amino” or substituted amine denotes the group NRR′, where R and R′ may independently by hydrogen, lower alkyl, substituted lower alkyl, aryl, substituted aryl, hetaryl, or substituted heteroaryl as defined herein, acyl or sulfonyl.

“Amido” denotes the group —C(O)NRR′, where R and R′ may independently by hydrogen, lower alkyl, substituted lower alkyl, aryl, substituted aryl, hetaryl, substituted hetaryl as defined herein.

“Carboxyl” denotes the group —C(O)OR, where R is hydrogen, lower alkyl, substituted lower alkyl, aryl, substituted aryl, hetaryl, and substituted hetaryl as defined herein.

“Aryl”—alone or in combination means an aromatic group which has at least one ring having a conjugated pi electron system and includes both carbocyclic aryl (e.g. phenyl) and heterocyclic aryl groups (e.g. pyridine), for example, phenyl or naphthyl optionally carbocyclic fused with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

The term “carbocyclic” refers to a compound which contains one or more covalently closed ring structures, and where the atoms forming the backbone of the ring are all carbon atoms. The term thus distinguishes carbocyclic from “heterocyclic” rings in which the ring backbone contains at least one atom which is different from carbon.

“Substituted aryl” refers to aryl optionally substituted with one or more functional groups, e.g., halogen, lower alkyl, lower alkoxy, alkylthio, acetylene, amino, amido, carboxyl, hydroxyl, aryl, aryloxy, heterocycle, heteroaryl, substituted heteroaryl, nitro, cyano, thiol, sulfamido and the like.

“Heterocycle” refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl or benzo[b]thienyl) and having at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heteroaryl”—alone or in combination means an aryl group which contains at least one heterocyclic ring, preferably a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group O, S, and N, and optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. Examples of heteroaryl groups are pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl and the like. A substituted heteroaryl contains a substituent attached at an available carbon or nitrogen to produce a stable compound.

“Heterocyclyl”—alone or in combination means a non-aromatic cycloalkyl group having from 5 to 10 atoms in which from 1 to 3 carbon atoms in the ring are replaced by heteroatoms of O, S or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. Examples of heterocyclyl groups are tetrahydrofuranyl, dihydropyridinyl, piperidinyl, pyrrolidinyl, piperazinyl, dihydrobenzofuryl, dihydroindolyl, and the like. A substituted hetercyclyl contains a substituent nitrogen attached at an available carbon or nitrogen to produce a stable compound.

“Substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Aralkyl” refers to the group —R—Ar where Ar is an aryl group and R is lower alkyl or substituted lower alkyl group. Aryl groups can optionally be unsubstituted or substituted with, e.g., halogen, lower alkyl, alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Heteroalkyl” refers to the group —R-Het where Het is a heterocycle group and R is a lower alkyl group. Heteroalkyl groups can optionally be unsubstituted or substituted with e.g., halogen, lower alkyl, lower alkoxy, alkylthio, acetylene, amino, amido, carboxyl, aryl, aryloxy, heterocycle, substituted heterocycle, hetaryl, substituted hetaryl, nitro, cyano, thiol, sulfamido and the like.

“Heteroarylalkyl” refers to the group -R-HetAr where HetAr is an heteroaryl group and R lower alkyl or substituted lower alkyl. Heteroarylalkyl groups can optionally be unsubstituted or substituted with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloalkyl” refers to a divalent cyclic or polycyclic alkyl group containing 3 to 15 carbon atoms.

“Substituted cycloalkyl” refers to a cycloalkyl group comprising one or more substituents with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloheteroalkyl” refers to a cycloalkyl group wherein one or more of the ring carbon atoms is replaced with a heteroatom (e.g., N, O, S or P).

“Substituted cycloheteroalkyl” refers to a cycloheteroalkyl group as herein defined which contains one or more substituents, such as halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloalkyl” denotes the group —R-cycloalkyl where cycloalkyl is a cycloalkyl group and R is a lower alkyl or substituted lower alkyl. Cycloalkyl groups can optionally be unsubstituted or substituted with e.g. halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloheteroalkyl” denotes the group —R-cycloheteroalkyl where R is a lower alkyl or substituted lower alkyl. Cycloheteroalkyl groups can optionally be unsubstituted or substituted with e.g. halogen, lower alkyl, lower alkoxy, alkylthio, amino, amido, carboxyl, acetylene, hydroxyl, aryl, aryloxy, heterocycle, substituted heterocycle, hetaryl, substituted hetaryl, nitro, cyano, thiol, sulfamido and the like.

The term “amine” refers to a chemical moiety of formula NR₁R₂ where R₁ and R₂ are independently selected from the group consisting of hydrogen, saturated or unsaturated alkyl, and homocyclic or heterocyclic ring moieties, where the ring is optionally substituted with one or more substituents independently selected from the group consisting of alkyl, halogen, trihalomethyl, carboxylate, nitro, and ester moieties.

The term “ketone” refers to a chemical moiety with formula —(R)ₙCOR′, where R and R′ are selected from the group consisting of saturated or unsaturated alkyl and homocyclic or heterocyclic ring moieties and where n is 0 or 1.

The term “carboxylic acid” refers to a chemical moiety with formula —(R)ₙCOOH, where R is selected from the group consisting of saturated or unsaturated alkyl and homocyclic or heterocyclic ring moieties, and where n is 0 or 1.

The term “alcohol” refers to a chemical substituent of formula —ROH, where R is selected from the group consisting of saturated or unsaturated alkyl, and homocyclic or heterocyclic ring moieties, where the ring moiety is optionally substituted with one or more substituents independently selected from the group consisting of alkyl, halogen, trihalomethyl, carboxylate, nitro, and ester moieties.

The term “ester” refers to a chemical moiety with formula —(R)ₙCOOR′, where R and R′ are independently selected from the group consisting of saturated or unsaturated alkyl and homocyclic or heterocyclic ring moieties and where n is 0 or 1.

The term “alkoxy” refers to a chemical substituent of formula —OR, where R is hydrogen or a saturated or unsaturated alkyl moiety.

The term “amide” refers to a chemical substituent of formula —NHCOR, where R is selected from the group consisting of hydrogen, alkyl, hydroxyl, and homocyclic or heterocyclic ring moieties, where the ring is optionally substituted with one or more substituents independently selected from the group consisting of alkyl, halogen, trihalomethyl, carboxylate, nitro, or ester.

The term “aldehyde” refers to a chemical moiety with formula —(R)ₙCHO, where R is selected from the group consisting of saturated or unsaturated alkyl and homocyclic or heterocyclic ring moieties and where n is 0 or 1.

The term “sulfone” refers to a chemical moiety with formula —SO₂R, where R is selected from the group consisting of saturated or unsaturated alkyl and homocyclic or heterocyclic ring moieties.

The term “phenylalkyl” means the aforementioned alkyl groups substituted by a phenyl group such as benzyl, phenethyl, phenopropyl, 1-benzylethyl, phenobutyl and 2-benzylpropyl.

The term “hydroxy-alkyl” means the aforementioned alkyl groups substituted by a single hydroxyl group such as 2-hydroxyethyl, 2-hydroxypropyl, 3-hydroxypropyl, 4-hydroxybutyl, 1-hydroxybutyl and 6-hydroxyhexyl.

The terms “alkylthio, alkenylthio, alkynylthio, alkylthio, hydroxy-alkylthio and phenyl-alkylthio” as used herein mean the aforementioned alkyl, alkenyl, alkynyl, hydroxy-alkyl and phenyl-alkyl groups linked through a sulfur atom to group R.

The term “substituted” as used herein means that the group in question, e.g., alkyl group, aryl group, etc., can bear one or more substituents including but not limited to halogen, hydroxy, cyano, amino, nitro, mercapto, carboxy and other substituents known to those skilled in the art.

The terms “saturated” as used herein means an organic compound with neither double or triple bonds. The term “unsaturated” as used herein means an organic compound containing either double or triple bonds.

In various embodiments it may be desirable to modify all scaffolds that bind, or only those that bind with a particular affinity, for example, only those that bind with high affinity, moderate affinity, low affinity, very low affinity, or extremely low affinity. It may also be advantageous to perform modification on a selection of scaffolds that bind with any combination of affinities.

The person of ordinary skill in the art will realize that the principles described herein can be applied to any interactions between molecules that participate in any binding reaction. Preferably, a small molecule will be one of the binding partners. But protein-ligand, protein-protein, protein-nucleic acid interactions, protein-carbohydrate interaction, or binding between any two molecules can be enhanced or improvements designed into the reaction using the principles described herein.

Identification of Binding Characteristics of Binding Compounds

It can also be beneficial in selecting compounds for testing to first identify binding characteristics that a ligand should advantageously possess. This can be accomplished by analyzing the interactions that a plurality of different binding compounds have with a particular target, e.g., interactions with one or more conserved residues in the binding site. These interactions are identified by considering the nature of the interacting moieties. In this way, atoms or groups that can participate in hydrogen bonding, polar interactions, charge-charge interactions, and the like are identified based on known structural and electronic factors.

Identification of Energetically Allowed Sites for Attachment

In addition to the identification and development of ligands, determination of the orientation of a molecular scaffold or other binding compound in a binding site allows identification of energetically allowed sites for attachment of the binding molecule to another component. For such sites, any free energy change associated with the presence of the attached component should not destablize the binding of the compound to the target to an extent that will disrupt the binding. Preferably, the binding energy with the attachment should be at least 4 kcal/mol., more preferably at least 6, 8, 10, 12, 15, or 20 kcal/mol. Preferably, the presence of the attachment at the particular site reduces binding energy by no more than 3, 4, 5, 8, 10, 12, or 15 kcal/mol.

In many cases, suitable attachment sites will be those that are exposed to solvent when the binding compound is bound in the binding site. In some cases, attachment sites can be used that will result in small displacements of a portion of the enzyme without an excessive energetic cost. Exposed sites can be identified in various ways. For example, exposed sites can be identified using a graphic display or 3-dimensional model. In a graphic display, such as a computer display, an image of a compound bound in a binding site can be visually inspected to reveal atoms or groups on the compound that are exposed to solvent and oriented such that attachment at such atom or group would not preclude binding of the enzyme and binding compound. Energetic costs of attachment can be calculated based on changes or distortions that would be caused by the attachment as well as entropic changes.

Many different types of components can be attached. Persons with skill are familiar with the chemistries used for various attachments. Examples of components that can be attached include, without limitation: solid phase components such as beads, plates, chips, and wells; a direct or indirect label; a linker, which may be a traceless linker; among others. Such linkers can themselves be attached to other components, e.g., to solid phase media, labels, and/or binding moieties.

The binding energy of a compound and the effects on binding energy for attaching the molecule to another component can be calculated approximately by manual calculation, or by using any of a variety of available computational virtual assay techniques, such as docking or molecular dynamics simulations. A virtual library of compounds derived from the attachment of components to a particular scaffold can be assembled using a variety of software programs (such as Afferent, MDL Information Systems, San Leandro, Calif. or CombiLibMaker, Tripos Associates, St. Louis, Mo.). This virtual library can be assigned appropriate three dimensional coordinates using software programs (such as Concord, Tripos Associates, St. Louis, Mo. or Omega, Openeye Scientific Software, Santa Fe, N. Mex.). These structures may then be submitted to the appropriate computational technique for evaluation of binding energy to a particular target molecule. This information can be used for purposes of prioritizing compounds for synthesis, for selecting a subset of chemically tractable compounds for synthesis, and for providing data to correlate with the experimentally determined binding energies for the synthesized compounds.

The crystallographic determination of the orientation of the scaffold in the binding site specifically enables more productive methods of assessing the likelihood of the attachment of a particular component resulting in an improvement in binding energy. Such an example is shown for a docking-based strategy in Haque et al Journal of Medicinal Chemistry 42:1428-40, 1999, wherein an “Anchor and Grow” technique which relied on a crystallographically determined fragment of a larger molecule, potent and selective inhibitors were rapidly created. The use of a crystallographically characterized small molecule fragment in guiding the selection of productive compounds for synthesis has also been demonstrated in Boehm et al, Journal of Medicinal Chemistry 43:2664-74, 2000. An illustration of the use of crystallographic data and molecular dynamics simulations in the prospective assessment of inhibitor binding energies can be found in Pearlman and Charifson, Journal of Medicinal Chemistry 44, 3417-23, 2001. Another important class of techniques which rely on a well defined structural starting point for computational design is the combinatorial growth algorithm based systems, such as the Grow Mol program (Bohacek and McMartin, Journal of the American Chemical Society 116:5560-71, 1994. These techniques have been used to enable the rapid computational evolution of virtual inhibitor computed binding energies, and directly led to more potent synthesized compounds whose binding mode was validated crystallographically (see Organic Letters (2001) 3(15):2309-2312).

Linkers

Linkers suitable for use in the invention can be of many different types. Linkers can be selected for particular applications based on factors such as linker chemistry compatible for attachment to a binding compound and to another component utilized in the particular application. Additional factors can include, without limitation, linker length, linker stability, and ability to remove the linker at an appropriate time. Exemplary linkers include, but are not limited to, hexyl, hexatrienyl, ethylene glycol, and peptide linkers. Traceless linkers can also be used, e.g., as described in Plunkett, M. J., and Ellman, J. A., 1995, J. Org. Chem., 60:6006.

Typical functional groups, that are utilized to link binding compound(s), include, but not limited to, carboxylic acid, amine, hydroxyl, and thiol. (Examples can be found in Solid-supported combinatorial and parallel synthesis of small molecular weight compound libraries; Tetrahedron organic chemistry series Vol.17; Pergamon, 1998; p85).

Labels

As indicated above, labels can also be attached to a binding compound or to a linker attached to a binding compound. Such attachment may be direct (attached directly to the binding compound) or indirect (attached to a component that is directly or indirectly attached to the binding compound). Such labels allow detection of the compound either directly or indirectly. Attachment of labels can be performed using conventional chemistries. Labels can include, for example, fluorescent labels, radiolabels, light scattering particles, light absorbent particles, magnetic particles, enzymes, and specific binding agents (e.g., biotin or an antibody target moiety).

Solid Phase Media

Additional examples of components that can be attached directly or indirectly to a binding compound include various solid phase media. Similar to attachment of linkers and labels, attachment to solid phase media can be performed using conventional chemistries. Such solid phase media can include, for example, small components such as beads, nanoparticles, and fibers (e.g., in suspension or in a gel or chromatographic matrix). Likewise, solid phase media can include larger objects such as plates, chips, slides, and tubes. In many cases, the binding compound will be attached in only a portion of such an objects, e.g., in a spot or other local element on a generally flat surface or in a well or portion of a well.

### EXAMPLES

The examples below are illustrative of the present invention, including the identification and description of scaffold groups for particular targets. However, it should be recognized that the various aspects of the invention can be carried out in a number of different ways. An example involving a particular target, the kinase PIM-1, is described in U.S. Application 60/411,398, and is incorporated herein in its entirety, including drawings. Selected information from that application is included in the examples below. Likewise, exemplary information is provided concerning another kinase target, PYK2.

### Example 1

### Scaffold Discovery

In an initial selection of a screening library, 6,347 compounds were obtained on the commercial market (Maybridge, Cornwall, England; Chembridge, San Diego, Calif.) and were selected for bias toward scaffold-like properties (having the properties of MW<350, clogP=3, and number of rotatable bonds less than 4) and were screened at 100 μM concentrations of each compound with a radioisotope Ser/Thr kinase enzymatic assay, using the lyn kinase assay. The assay was based on the extent of phosphorylation of the enzyme substrate with detection performed using an antibody to the phosphorylated substrate.

Of the 6,347 scaffold-like compounds screened, 10% or more resulted in a signal about twice that of sigma of the background signal. The number of compounds obtained was less than 500, so the compounds did not need to be clustered based on chemical similarity. In cases where large numbers of active compounds are found, e.g., more than 500 compounds are active, active compounds may be clustered into chemical families based on the similarity of substructures contained within those compounds. 500 compounds (or other convenient number) can be conveniently chosen as representative members for high-throughput co-crystallization and co-crystallography structure determinations. One manner of selecting the representative compounds can be using a suitable software program such as, e.g., ISIS® software (Molecular Design Labs, San Leandro, Calif.).

The 500 compounds were added at 100 μM concentrations to 5 mg of the purified protein in a 96 well format and were concentrated to a protein concentration of 5 mg/ml-15 mg/ml. Each sample was subjected to a crystallization trial under 96 different protein crystallization conditions. The compounds that crystallized with the protein were harvested as co-crystals and exposed to X-rays in an X-ray diffraction experiment in order to collect a unique set of data. Using these data one can determine the crystal structures of the compounds bound to the proteins, and create a model of the interaction between the compound and the protein target. These structures provide the basis for 1) determining which of these compounds have the most advantageous structures for exploring the unoccupied regions of the molecular space in the protein binding pocket in order to enhance binding, selectivity, or other desirable physical properties of the compounds themselves; and 2) these structures can also be utilized for making decisions and guiding the chemistry in a rational or focused method. Thus, one efficiently uses the chemical resource to synthesize compounds that are drug “lead” compounds that interact specifically with individual members of the protein family.

Software is available for performing the compound clustering based on chemical structure similarity, using a measure such as the Tanimoto coefficient. Thus, this step may be conveniently performed using, for example, ISIS® software (Molecular Design Labs, (San Leandro, Calif.). Alternatively, other commonly used types of molecular clustering can be easily used in other software packages (Unity, Tripos Associates St. Louis Mo. and MOE, Chemical Computing Group, Montreal, Quebec, Canada).

### Example 2

### Scaffold Discovery

975 compounds biased toward scaffold-like properties (as described in Example 1) were screened at 100 μM concentrations of each compound with an enzymatic assay. The assay was based on the amount of ATP left in the sample as determined by a luciferase fluorescence signal. Of the 975 compounds screened, 101 compounds gave a signal of twice the background signal shown by controls and the non-biased compounds.

The 101 compounds were then added at 100 μM concentrations to 5 mg of the purified protein in a 96 well format and concentrated to a protein concentration of between mg/ml-15 mg/ml. Each sample was then subjected to a co-crystallization trial under 96 different co-crystallization conditions. The compounds that co-crystallize with the protein was harvested as co-crystals. The co-crystals were then exposed to X-rays through X-ray diffraction to collect data that enables the definitive determination of the crystal structures of the compounds bound to the proteins. These structures therefore provide the basis for 1) determining which compounds have the most advantageous chemical structures for exploring the unoccupied regions of molecular space in the binding pocket for enhancing binding, selectivity, and desirable physical properties of the compounds themselves; and 2) these structures can also be used to make decisions guiding the scaffold design in a rational or focused manner that efficiently uses the physical and chemical data to synthesize drug “lead” compounds for specifically interacting with individual members of the protein family.

### Example 3

### Assaying Compounds for Binding Activity With Protein(s) of Interest

The person of ordinary skill will realize assays suitable for identifying compounds that bind to the protein(s) of interest. One assay that can be used is a lyn kinase assay. His-Lyn (M16038, kinase domain) can be produced in HEK293T cells by transient expression. His-Lyn can be captured on a metal-chelate plate (96 well). The kinase reaction can be carried out in 100 μl wells with HEPES 50 mM, pH 7.2, 15 mM NaCl, 2 μM ATP, 1 ug/well bovine serum albumin, at 37 C for one hour. The compounds can be diluted with DMSO by a ratio of 1:2, with a final concentration of DMSO of 1% in all wells. A positive control (PC) can be 4 wells with ATP and DMSO, and a negative control (NC) can be 4 wells without ATP. Phospho-tyrosine is detected by PT20-HRP (Santa Cruz Biotech, Santa Cruz, Calif.) at 1:2000. Bounded PY20—HRP is measured by TMB (3,3′, 5,5′-tetramethyl benzidine) conversion. The data can be processed as % control=[(treatments−NC)/(PC−NC)]×100. IC₅₀ of an inhibitor is estimated at the concentration where the percent of inhibition reaches 50%.

### Example 4

### Csk and Pyk2 Low Affinity Binding Assays

In other embodiments, compounds were assayed for activity with Csk and Pyk2. Human Csk is a non-receptor protein tyrosine kinase. One of its major functions in vivo is to specifically phosphorylate a conserved C-terminal tyrosine on the proto-oncogene Src and Src family members (including Lck, Fyn, Yes etc.) and down-regulate them by shutting off their kinase activity. In this way, Csk plays a role in cell growth and differentiation in a variety of tissues including the immune system, bone, and the nervous system.

Proline-rich tyrosine kinase 2 (Pyk2) is a non-receptor tyrosine kinase which is a homologue of focal adhesion kinase (FAK). Pyk 2 is expressed in the central nervous system and blood cells. Elevation of intracellular calcium and protein kinase C activation leads to the autophosphorylation of Pyk2 and the formation of a complex with protein tyrosine kinase Src.

Csk (and Pyk2 in a separate assay) were mixed with poly(Glu:Tyr) and coated on an ELISA plate in kinase buffer. Test compounds were added to each well at a concentration of about 200 uM. ATP was added to a final concentration of 2 uM. After 1 hour of incubation at 37 C, phospho-Tyr was detected with ELISA solution. Color development of TMB was measured at 650 nm. The Csk was the (His)₆-kinase domain fusion, and the Pyk2 was the kinase domain-(His)₆ fusion, both produced in BL21 E. coli. The kinase buffer was 50 mM HEPES, pH 7.2 with 100 uM of manganese. The ELISA solution was 1:1000 at 5% milk.

The person of ordinary skill in the art will realize that a variety of assays are available and suitable for use in the present invention, or that the assays described herein can be modified when desirable and in ways known to the person of ordinary skill.

### Example 5

### HisP Low Affinity Binding Assay

One class of ABC transporter inhibitor acts by competing for the binding of the substrate, ATP, to the nucleotide binding domain, his P. The following methods enable screens of such inhibitors at low affinity.

Cloning of the hisP Gene from Salmonella typhimurium and Expression of the Encoded Protein in Bacteria.

Common genetic engineering methods were applied to make a plasmid vector designed for over-expression of hisP in bacteria. Two oligonucleotides were synthesized suitable for priming PCR reactions to amplify the his P gene from Salmonella typhimurium, using the genomic DNA of this organism available from the American Type Culture Collection. The oligonucleotides designed for synthesizing the coding strand of the gene was designed to add a flanking NdeI site that encodes the codon for the initiating Met residue. The oligonucleotide designed for synthesizing the non-coding strand was designed to replace the termination codon with a SalI hexamer restriction site encoding the residues Glu-Val. The resulting PCR product was cleaved with NdeI and SalI restriction enzymes, and the cleaved product ligated to complementary sites of C-terminal His-tagged glutathione S-transferase (GST) expression vector under a lac-regulated promoter. The final vector encodes the full-length his P protein fused with an N-terminal GST and with a C-terminal tail of Glu-Val-His-His-His-His-His-His.

Preparation of hisP from E. coli and Metal Affinity Purification in the Presence of Phosphate Buffer for Stabilization.

In order to effectively assay compounds that inhibit the ATPase activity of his P by competing for the binding of the substrate ATP, it is required to reduce the ATP concentration to sub-saturating levels. However the stability and solubility of the his P protein requires saturating doses of ATP. We discovered that his P can also be stably prepared in the presence of phosphate-containing buffer at zero ATP concentrations, as it is the phosphate moieties that appear to be the stabilizing feature of the ATP molecule.

One liter of bacterial strain BL21 (Novagen Inc.) harboring the GST-hisP-hexa His expression plasmid is grown at 22° C. in 2YT broth with 100 μg/ml Ampicillin antibiotic. At an OD₆₀₀ of 0.6, the inducer isopropyl-thiogalactopyranoside is added to a concentration of 0.1 mM, and the culture is continued for another 3-6 hours. The cells are harvested by centrifugation and the pellets flash-frozen in liquid N₂ and stored at −80 C.

The frozen pellets from 1 liter culture are thawed and suspended in 20 ml extraction buffer 1 (100 mM NaPO₄, pH8, 150 mM NaCl, 0.05% Tween 20, 10% glycerol, 0.5% monothioglycerol (MTG), and 0.1 mM phenylmethysulfonyl fluoride (PMSF)). To this is added 1 ml 10 mg/ml lysozyme, with incubation on ice for 15 min. The suspension is sonicated until the chromosomal DNA is sufficiently fragmented to create a non-viscous solution, approximately 1 minute. The solution is transferred to a polycarbonate centrifuge tube and centrifuged for 60 min at 20,000 rpm in a Sorvall SA20 rotor. The supematent is recovered into a separate tube.

2 ml buffer 1-washed slurry of Talon metal affinity resin (Clontech, Palo Alto, Calif.) is mixed with the E. coli supernatent with 2 mM imidazole added, and incubated at 4 C for 1 hour with constant mixing on a nutator. The resin is batch-washed by serially centrifuging the resin and resuspending, first in 10 ml extraction buffer 1+2 mM imidazole, secondly with 10 ml buffer 2 (100 mM NaPO₄, 100 mM NaCl, 20% glycerol, 0.5% MTG, 0.1 mM PMSF, and 2 mM imidazole), and thirdly with 5 ml buffer 3 (100 mM KPO₄, 20% glycerol, 0.5% MTG, 0.1 mM PMSF, and 2 mM imidazole). The centrifuged beads are then batch-eluted with 4 ml of buffer 4 (100 mM KPO₄, 20% glycerol, 0.5% MTG, 0.1 mM PMSF, and 100 mM imidazole). To the 4 ml of eluate is added EDTA to a final concentration of 0.1 mM, and DTT to a final concentration of 0.1 mM. The solution is concentrated in a centriprep filter concentrator (Centricon) to a concentration of 15 mg/ml, and this is flash-frozen in liquid N2 and stored at −80 C. The Talon™ metal affinity resin is a durable immobilized metal affinity chromatography (IMAC) resin that uses cobalt ions for purifying recombinant polyhistidine-tagged proteins. The matrix is made of Sepharoseg 6B-CL with 6% cross-linked agarose, with a bead size of 45-165 μm and a maximum linear flow rate of 75-150 cm/h.

Assay of the ATPase Activity of hisP In Vitro Using Firefly Luciferase to Monitor the Amount of ATP Degraded, and Showing the Effects of Compounds That Inhibit the ATPase

The most commonly used system to assay ATPase activity monitors the production of inorganic phosphate. However, this is not possible when phosphate buffers are used to stabilize the enzyme. Instead one may employ the highly-sensitive enzyme, firefly luciferase, to assay the amount of ATP remaining after the incubation of the his P ATPase. This method is quite suitable for the screening of potential inhibitor compounds because of three properties: 1) the luciferase has an extremely wide dynamic range with low background, yielding a signal over background in this step of up to 1,000,000-fold), 2) the luciferase assay is very simply set up and read in a luminometer, thus making it suitable for high-throughput screening, and 3) inhibitory compounds give rise to an increased signal, thus decreasing the possibilities of false-positives from non-specific interference effects.

The compounds of interest are dissolved in DMSO at 100× strength, and 1 ul each transferred to a single well of a 96-well microtiter plate. As a control, DMSO lacking any compound is dispensed into separate wells of the plate. To these are added 50 μl of a mixture containing 60 mM MOPS buffer pH 7.0, 4% glycerol, 2 mM MgCl₂, 200 μM ATP. Then 50 μl of a mixture containing 60 mM MOPS pH 7.0, 4% glycerol, 600 μg/ml GST-his P-hexaHis is added, and the mixture shaken for 1 min. and incubated 1.5 hours at 37 C. Following the incubation, 4 μl of the reaction mix is transferred to 100 μl in another 96-well plate of Luciferase ATP assay mix (Roche), diluted 1:10 from the manufacturers protocol in 40 mM Tris buffer pH 7.8. The plates are then read using a luminometer Wallac 1420 multilabel counter.

Quantification of the Relative Capacities of Compounds to Occupy the ATP Binding Site of hisP by Measurement of Their Abilities to Block the Binding of a Fluorescent Analog of ATP in a Fluorescence Polarization Assay

The fluorescent derivative of ATP (Fluorescein-N₆-ATP, catalog NEL-502, NEN Life Sciences, Boston, Mass.) binds to his P to give a polarized fluorescence signal. This signal is diminished in a concentration-dependent manner by compounds that bind to hisP at the ATP-binding site. This observation can be utilized to quantify the relative binding affinities of compounds of potential interest as competitive inhibitors of his P.

The compounds of interest are dissolved in DMSO and serially diluted in DMSO to obtain solutions that differ in each compounds concentration. 1 μl of each dilution is transferred to separate wells of a 96-well microtiter plate. To each is added 100 μl of a reaction mixture containing 60 mM MOPS buffer pH 7.0, 4% glycerol, 0.1 mM EDTA, 1 mM MgCl₂, 2.5 nM Fluorescein-N₆-ATP, and 15 ug/ml GST-his P-hexaHis, and the signal read in a multilabel counter.

### Example 7

### Cloning of PIM-1

An exemplary application of the present invention involved the kinase PIM-1 as target. The PIM-1 DNA encoding amino acids 1-313 and 29-313 was amplified from human brain cDNA (Clonetech) by PCR protocols and cloned into a modified pET 29 vector (Novagen) between NdeI and SalI restriction enzyme sites. The amino acid sequences of the cloned DNA were confirmed by DNA sequencing and the expressed proteins contain a hexa-histidine sequence at the C terminus. The protein was expressed in E. coli BL21(DE3)pLysS (Novagen). The bacteria were grown at 22° C. in Terrific broth to 1-1.2 OD600 and protein was induced by 1 mM IPTG for 16-18 h. The bacterial pellet was collected by centrifugation and stored at −70° C. until used for protein purification. PIM-2 and PIM-3 are cloned similarly.

### Example 8

### Purification of PIM-1

The bacterial pellet of approximately 250-300 g (usually from 16 L) expressing PIM-1 kinase domain (29-313) was suspended in 0.6 L of Lysis buffer (0.1 M potassium phosphate buffer, pH 8.0, 10% glycerol, 1 mM PMSF) and the cells were lysed in a French Pressure cell at 20,000 psi. The cell extract was clarified at 17,000 rpm in a Sorval SA 600 rotor for 1 h. The supernatant was re-centrifuged at 17000 rpm for another extra hour. The clear supernatant was added with imidazole (pH 8.0) to 5 mM and 2 ml of cobalt beads (50% slurry) to each 40 ml cell extract. The beads were mixed at 4° C. for 3-4 h on a nutator. The cobalt beads were recovered by centrifugation at 4000 rpm for 5 min. The pelleted beads were washed several times with lysis buffer and the beads were packed on a Biorad disposable column. The bound protein was eluted with 3-4 column volumes of 0.1 M imidazole followed by 0.25 M imidazole prepared in lysis buffer. The eluted protein was analyzed by SDS gel electrophoresis for purity and yield.

The eluted protein from cobalt beads was concentrated by Centriprep-10 (Amicon) and separated on Pharmacia Superdex 200 column (16/60) in low salt buffer (25 mM Tris-HCl, pH 8.0, 150 mM NaCl, 14 mM beta mercaptoethanol). The peak fractions containing PIM-1 kinase was further purified on a Pharmacia Source Q column (10/10) in 20 mM Tris-HCl pH 7.5 and 14 mM beta mercaptoethanol using a NaCl gradient in an AKTA-FPLC (Pharmacia). The PIM-1 kinase eluted approximately at 0.2 M NaCl gradient. The peak fractions were analyzed by SDS gel electrophoresis and were pooled and concentrated by Centriprep 10. The concentrated PIM-1 protein (usually 50-60 A280/ml) was aliquoted into many tubes (60 ul), flash frozen in liquid nitrogen and stored at −70° C. until used for crystallization. The frozen PIM-1 kinase still retained kinase activity as concluded from activity assays. PIM-2 and PIM-3 can be purified in the same way with small adjustments to conditions, e.g., elution conditions.

### Example 9

### Crystallization of PIM-1

PIM-1 Protein Crystal Growth:

All materials were purchased through Hampton Research, Inc. (Laguna Niguel, Calif.) unless otherwise noted. PIM-1 protein @ 7 and 14 mg/ml was screened against Hampton Crystal Screen 1 and 2 kits (HS 1 and HS2) and yielded successful crystals growing in at least 10 conditions from HS 1 alone. Crystals were grown initially using sitting drops against the Hampton screening conditions set in Greiner 96 well CrystalQuick crystallization plates with 100 ul reservoir and 1 ul protein+1 ul reservoir added per platform (1 of 3 available). Conditions from Hampton Screen 1 yielded obvious protein crystals in conditions: #2,7,14,17,23,25,29,36,44, and 49. These crystals were grown at 4° C., and grew in size to varying dimensions, all hexagonal rod shaped and hardy.

Crystals of larger dimensions, 100 uM wide×400 uM long, were then grown in larger drop volumes and in larger dimension plates. Refined grids were performed with both hanging and sitting drop methods in VDX plates (cat. # HR3-140) or CrysChem plates (cat. # HR3-160). There appeared to be no obvious difference of crystal size or quality between the two methods, but there was a preference to use hanging drops to facilitate mounting procedures.

We proceeded with refining conditions by gridding 4 independent reservoir conditions initially obtained from the screening kits.

1) HS1 ft 17 was optimized to 0.2 M LiCl, 0.1 M Tris pH 8.5 and 5%-15% Polyethylene glycol 4000;

2) HS1 # 25 was optimized to 0.4 M-0.9 M Sodium Acetate trihydrate pH 6.5 and 0.1 M Imidazole;

3) HS1 # 29 was optimized to 0.2M-0.7 M Sodium Potassium tartrate and 0.1 M MES buffer pH 6.5;

4) HS1 # 44 was optimized to 0.25 M Magnesium formate.

These optimized conditions produced crystals with the most consistent size and quality of appearance. Conditions were further evaluated by x-ray diffraction analysis of the resulting protein crystals, and keeping in mind the utility for forming compound co-crystals in these conditions as well (ie. salt composition and concentration effects are important to develop suitable compound solubility in the crystallization experiments). Native crystals grew as rods in many drops to large dimensions of approximately 100 um wide and 500 um long.

Seleno Methionine labeled PIM-] protein crystal growth.

Se-Met labeled PIM-1 protein was expressed and purified as described by Hendrickson, W. A., and Ogata, C. M. (1997) “Phase determination from multiwavelength anomalous diffraction measurements, Methods Enzymol., 276, 494-523, and Hendrickson, W. A., Horton, J. R., and LeMaster, D. M. (1990) “Selenomethionyl proteins produced for analysis by multiwavelength anomalous diffraction (MAD): a vehicle for direct determination of three-dimentional structure, EMBO J., 9, 1665-1672. This preparation appeared to be less soluble as evidenced by more pronounced nucleation within the screen drops and due to the hydrophobic nature of Se labeled proteins. Crystals grew small and in showers compared to the previously evaluated similar drop conditions that the native protein grew well in. Upon finer gridding, 20 μm wide×100 μm long crystals were obtained in condition HS1 # 17 optimized at 0.2 M LiCl, 0.1 M Tris pH 8.5 and 5%-15% PEG 4000. These crystals and all others were carefully mounted in 50-100 uM nylon loops on copper stem magnetic bases that were flash frozen in liquid nitrogen in appropriate cryogenic buffer and taken to the Lawerence Berkeley Lab synchrotron, the Advanced Light Source (ALS) beamline 8.3.1.

PIM-1 Protein/Molecular Scaffolds Co-Crystal Growth:

In order to add compounds to PIM-1 protein, compounds were added directly from their DMSO stocks (20-200 mM) into the protein solution at high concentration. The procedure involved adding the DMSO stocks containing compound as a thin layer to the wall of the 1.5 ml eppendorf tube that contains the protein. The solution was then gently rolled over the wall of the tube until the compound was in the protein solution. The final concentration of compounds in the PIM-1 solution usually achieved was between 0.5 and 1 mM with DMSO concentrations less than 2% being added. The solutions were then set-up in trays immediately as previously described.

PIM-1/Compound Co-Crystal Screening in HS1:

Two conditions for crystal growth have resulted in the best results with PIM-1 protein and added compounds. The optimized Na—K tartrate and Na-acetate tetrahydrate solutions listed above. Crystals varied greatly in size but data has been collected on various crystals that are between 20 uM and 100 uM in width. These crystals were typically several hundred microns long and some required manipulation as well as being broken to facilitate mounting procedures into loops.

### Example 10

### Diffraction Analysis of PIM-1

Crystals were first determined to diffract on a Rigaku RU-200 rotating copper anode x-ray source equipped with Yale focusing optics and an R-AXIS 2C imaging plate system. A crystal grown in the optimized condition HS1 # 17 (DY plate Dec. 14, 2001) was used to conduct initial diffraction experiments.

After x-ray diffraction was initially determined as described above, large native protein crystals grown in Mg-Formate (DY plate) and were frozen in cryoprotectant by submersion in liquid nitrogen and then tested for diffraction at ALS beamline 8.3.1. Data was originally collected, indexed and reduced using Mosflm. The spacegroup was determined to be P65.

We have collected 3 native data sets, the highest resolution obtained with good statistics after merging is to 2.0 angstroms.

We have collected a MAD data set on the Se-Met labeled PIM-1 crystal using the experimentally determined 12668 eV peak and 11000 eV remote for selenium to 3.2 angstroms. Subsequently a 2.6 angstrom Se peak data set was collected at the experimentally determined peak of 12668 eV radiation.

We have collected more than 50 PIM-1/binding compound co-crystal data sets. All data was indexed and reduced as indicated in the computational crystallographic work that follows.

PIM-1 Structure Determination and Refinement

Data Set: Native, Resolution: 2.13

The primary structure determination was carried out using Molecular Replacement method with programs

EPMR (Public domain)

AmoRe (from CCP4))

And a homology model of PIM-1 based on the protein Phosphorylase Kinase (PDB ID: 1PHK—Owen et al., 1995, Structure 3:467)

The molecular replacement was carried out in all of the P6 space groups (P61, P62, . . . P65). The best solution was obtained in P65.

The molecular replacement solution was improved by several rounds of the cycles of

Model Building in O (from DatOno AB)

Annealing in CNX (from Accelerys)

SigmaA weighting and Solvent Flattening the resultant map with DM (from CCP4)

The statistics at the end of these cycles were R ˜36%.

Data set: SeMet (2 wavelengths), Resolution: 3.3

The MAD phased data (with SOLVE (from Los Alamos National Laboratory)) helped improve the model in the refinement with REFMAC (from CCP4).

Data set: SeMet (1 wavelength), Resolution: 2.6

Further improvement of the model was obtained using SAD Phasing with SOLVE and subsequent improvement with RESOLVE produced an excellent map into which the PIM1 model could be rebuilt completely.

The newly built model refined with CNX/Anneal and then with CCP4/Refmac=to give R=27.7% and Rfree=31.9%

Data set: Native, Resolution: 2.1

The above model has been further refined against the native data with CCP4/Refmac, giving R=22.1%, Rfree=24.2%.

Atomic coordinates for the native PIM-1 apo protein crystal are provided in Table 1.

### Example 11

### PIM-1 Co-Crystal Structures

Exemplary co-crystal structures have been determined for numerous compounds with PIM-1, using methods as generally described above. Those co-crystals include the following (the number indicates the compound id and the compound source is provided in parentheses):

PIM1_{—}5104579 (Chembridge)

PIM1_{—}5317991 (Chembridge)

PIM1_{—}5348396 (Chembridge)

PIM1_{—}5377348 (Chembridge)

PIM1_NRB02258 (Maybridge)

PIM1_NRB05093 (Maybridge)

PIM1_RJF00907 (Maybridge)

Also, PIM-2 was co-crystallized with AMPPNP. Atomic coordinates for the co-crystal are provided in Table 2.

### Example 12

### PIM Binding Assays

Such binding assays can be performed in a variety of ways, including a variety of ways known in the art. For example, competitive binding to PIM-1 can be measured on Nickel-FlashPlates, using His-tagged PIM-1 (˜100 ng) and ATPy[³⁵S] (˜10 nCi). As compound is added, the signal decreases, since less ATPy[³⁵S] is bound to PIM1 which is proximal to the scintillant in the FlashPlate. The binding assay can be performed by the addition of compound (10 μl; 20 mM) to PIM-1 protein (90 10 μl) followed by the addition of ATPγ[³⁵S] and incubating for 1 hr at 37° C. The radioactivity is measured through scintillation counting in Trilus (Perkin-Elmer).

Alternatively, any method which can measure binding of a ligand to the ATP-binding site can be used. For example, a fluorescent ligand can be used. When bound to PIM1, the emitted fluorescence is polarized. Once displaced by inhibitor binding, the polarization decreases.

Determination of IC50 for compounds by competitive binding assays. (Note that K₁ is the dissociation constant for inhibitor binding; K_{D} is the dissociation constant for substrate binding.) For this system, the IC50, inhibitor binding constant and substrate binding constant can be interrelated according to the following formula:

When using radiolabeled substrate

$${K}_{I} = \frac{IC50}{1 + [ {L}^{*} ] / {K}_{D}} ,$$

the IC50˜K₁ when there is a small amount of labeled substrate.

### Example 13

### PIM Activity Assays and Ligand Development

Inhibitory or exhitory activity of compounds binding to PIM-1 was determined using a kinase assay. A number of different assays for kinase activity can be utilized for assaying for active modulators and/or determining specificity of a modulator for a particular kinase or group or kinases. In addition to the assays mentioned below, one of ordinary skill in the art will know of other assays that can be utilized and can modify an assay for a particular application.

An assay for kinase activity that can be used for PIM kinases, e.g., PIM-1, can be performed according to the following procedure using purified kinase using myelin basic protein (MBP) as substrate. An exemplary assay can use the following materials: MBP (M-1891, Sigma); Kinase buffer (KB=HEPES 50 mM, pH7.2, MgCl₂:MnCl₂ (200 μM); ATP (γ-³³P):NEG602H (10 mCi/mL)(Perkin-Elmer); ATP as 100 mM stock in kinase buffer; EDTA as 100 mM stock solution.

Coat scintillation plate suitable for radioactivity counting (e.g., FlashPlate from Perkin-Elmer, such as the SMP200(basic)) with kinase+MBP mix (final 100 ng+300 ng/well) at 90-μL/well in kinase buffer. Add compounds at 1 μL/well from 10 mM stock in DMSO. Positive control wells are added with 1 μL of DMSO. Negative control wells are added with 2 μL of EDTA stock solution. ATP solution (10 μL) is added to each well to provide a final concentration of cold ATP is 2 μM, and 50 nCi ATPγ[³³P]. The plate is shaken briefly, and a count is taken to initiate count (IC) using an apparatus adapted for counting with the plate selected, e.g., Perkin-Elmer Trilux. Store the plate at 37° C. for 4 hrs, then count again to provide final count (FC).

Net 33P incorporation (NI) is calculated as: NI=FC−IC.

The effect of the present of a test compound can then be calculated as the percent of the positive control as: % PC=[(NI−NC)/(PC−NC)]×100, where NC is the net incorporation for the negative control, and PC is the net incorporation for the positive control.

As indicated above, other assays can also be readily used. For example, kinase activity can be measured on standard polystyrene plates, using biotinylated MBP and ATPγ[³³P] and with Streptavidin-coated SPA (scintillation proximity) beads providing the signal.

Additional alternative assays can employ phospho-specific antibodies as detection reagents with biotinylated peptides as substrates for the kinase. This sort of assay can be formatted either in a fluorescence resonance energy transfer (FRET) format, or using an AlphaScreen (amplified luminescentproximity homogeneous assay) format by varying the donor and acceptor reagents that are attached to streptavidin or the phosphor-specific antibody.

Exemplary compounds within Formula I, Formula II, and Formula III as described below were assayed for inhibitory activity with PIM-1. The ability to develop ligands is illustrated by 2 compounds from the quinolinone molecular scaffold group (Formula III). A compound with R1, R2, R3, R4, R5, and R6=H, had 100% inhibition of PIM-1 at 200 μM concentration, while a compound with R1 =phenyl group, R2, R3, R5, and R7=H, and R4=OCF₃, had only 3% inhibition of PIM-1 at 200 μM.

Another example of the ability to discover ligands is illustrated by two compounds from the 7-azaindole group (Formula II) and the co-crystal structures of these compounds with PIM-1. One compound (Formula III, R1=R2=R3=R4=R5=H) was found to be weakly active when assayed for inhibitory activity against PIM-1 (IC50>200 μM, 8% inhibition at 200 μM). Co-crystallography of this compound with PIM-1, results shown below left, revealed a binding mode for the scaffold placing it next to a hydrophobic contact surface, a feature which is present in this region in many kinases. The identification of an energetically allowed site of substitution for this scaffold could be determined using the DRY probe from the GRID program (Molecular Discovery Ltd. London UK) which indicated that two positions in Formula II (R2 and R3) allowed for substitution in energetically favorable regions consistent with interaction at this site. Addition of an analino subsitutent (R2=NH-Phenyl) resulted in a compound which was determined by co-crystallography to bind to PIM-1 in the expected fashion. This also resulted in an improvement in inhibitory activity against PIM-1 to 169 μM. This activity trend was also observed for the compounds in Pyk-2, where the inhibitory activity of the two compounds improved from an IC50>200 μM in the former to an IC50=21 μM in the latter.

### Example 14

### PIM-1 Scaffolds and Binding Compounds

A number of different molecular scaffolds that bind to PIM-1 have been identified based on demonstrated activity of simple compounds, validated with co-crystallography. Derivative PIM-1 binding compounds have been prepared, providing corresponding scaffold groups. Three such scaffolds and derivative compounds are described below.

One scaffold group is represented by compounds of Formula I. Compounds of this group have been validated by co-crystallography with PIM-1. The simplest scaffold has H at each of the R positions.

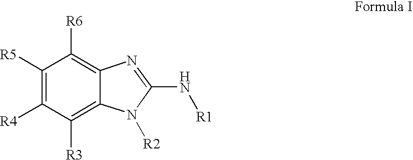

where:

R¹ is hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, —C(X)NR¹⁶R¹⁷, or —S(O₂)R

R² is hydrogen, trifluormethyl, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, C(X)NR¹⁶R¹⁷, or —S(O₂)R²¹;

R³ and R⁴ are independently hydrogen, hydroxy, fluorine, chlorine, trifluoromethyl, optionally substituted alkoxyl, optionally substituted thioalkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, or —S(O₂)R²¹;

R⁵ is hydrogen, hydroxyl, fluorine, chlorine, trifluoromethyl, optionally substituted lower alkoxy, optionally substituted lower thioalkoxy, optionally substituted amine, optionally substituted lower alkyl, —NR¹⁶C(X)NR¹⁶R¹⁷, —C(X)R²⁰, or —S(O₂)R²¹;

R⁶ is hydrogen, hydroxyl, fluorine, chlorine, optionally substituted lower alkoxy, optionally substituted lower thioalkoxy, or optionally substituted amine;

R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl;

R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R²¹ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

X=O, or S.

A second scaffold group with members validated in co-crystals with PIM-1 is provided by compounds of Formula II:

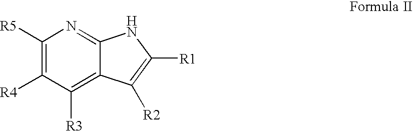

where:

R¹ is hydrogen, hydroxy, fluorine, chlorine, trifluoromethyl, optionally substituted alkoxyl, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —NR¹⁶C(X)NR R¹⁷, —C(X)R²⁰, or —S(O₂)R²¹;

R² is hydrogen, fluorine, chlorine, trifluoromethyl, optionally substituted alkoxyl, optionally substituted thioalkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —NR¹⁶C(X)NR¹⁶R¹⁷, —C(X)R²⁰, or —S(O₂)R²¹;

R³ and R⁴ are independently hydrogen, hydroxy, fluorine, chlorine, trifluoromethyl, optionally substituted alkoxyl, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —NR¹⁶C(X)NR¹⁶R¹⁷, —C(X)R²⁰, or —S(O₂)R⁷²¹;

R⁵ is hydrogen, fluorine, chlorine, trifluoromethyl, optionally substituted lower alkoxy, optionally substituted amine, optionally substituted lower alkyl, or —NR¹⁶C(X)NR¹⁶R¹⁷;

R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl;

R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R²¹ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

X=O or S.

A third scaffold group with members validated with PIM-1 is provided by compounds of formula III.

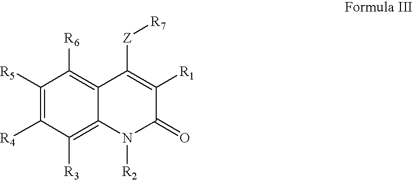

where:

Z=O, S, NR¹⁸, or CR¹⁸R⁹;

R¹ is hydrogen, hydroxyl, halogen, optionally substituted alkoxy, optionally substituted thioalkoxy, optionally substituted amine, optionally substituted aryl, optionally substituted aralkyl, optionally substituted heteroaryl, optionally substituted heteroaralkyl, —NR¹⁶R¹⁷S(O₂)R²¹ or —C(X)R²⁰;

R² is hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, or —S(O₂)R²¹;

R³ is hydrogen, hydroxyl, fluorine, chlorine, optionally substituted alkoxyl, optionally substituted amine, NR¹⁶C(X)NR¹⁶R¹⁷, —C(X)R²⁰, or —S(O₂)R²¹;

R⁴ is hydrogen, fluorine, chlorine, trifluoromethyl, optionally substituted lower alkoxy, optionally substituted amine, or optionally substituted lower alkyl;

R⁵ and R⁶ are independently hydrogen, hydroxyl, fluorine, chlorine, trifluoromethyl, optionally substituted alkoxyl, optionally substituted thioalkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, —C(X)R²⁰, or —S(O₂)R²¹;

R⁷ is hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl, or —C(X)R⁸;

R⁸ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R⁹ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R¹⁶ and R¹⁷ are independently hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, optionally substituted heteroaralkyl;

R¹⁸ is hydrogen, optionally substituted alkyl, optionally substituted lower alkenyl, optionally substituted lower alkylnyl, optionally substituted cycloalkyl, optionally substituted heterocycloalkyl, optionally substituted aryl, optionally substituted aralkyl, optionally substituted heteroaryl, or optionally substituted heteroaralkyl, C(X)R²⁰, C(X)NR¹⁶R¹⁷, or —S(O₂)R²¹;

R¹⁹ is hydrogen, optionally substituted alkyl, optionally substituted lower alkenyl, optionally substituted lower alkylnyl, optionally substituted cycloalkyl, optionally substituted heterocycloalkyl, optionally substituted aryl, optionally substituted aralkyl, optionally substituted heteroaryl, or optionally substituted heteroaralkyl, C(X)R²⁰, C(X)NR¹⁶R¹⁷, or —S(O₂)R²¹;

R²⁰ is hydroxyl, optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

R²¹ is optionally substituted lower alkoxy, optionally substituted amine, optionally substituted lower alkyl, optionally substituted lower alkenyl, optionally substituted lower alkynyl, optionally substituted cycloalkyl, optionally substituted heterocycloalkyl, optionally substituted aryl, optionally substituted aralkyl, optionally substituted heteroaryl, or optionally substituted heteroaralkyl;

X=O or S.

“Halo” or “Halogen”—alone or in combination means all halogens, that is, chloro (Cl), fluoro (F), bromo (Br), iodo (I).

“Hydroxyl” refers to the group —OH.

“Thiol” or “mercapto” refers to the group —SH.

“Alkyl”—alone or in combination means an alkane-derived radical containing from 1 to 20, preferably 1 to 15, carbon atoms (unless specifically defined). It is a straight chain alkyl, branched alkyl or cycloalkyl. Preferably, straight or branched alkyl groups containing from 1-15, more preferably 1 to 8, even more preferably 1-6, yet more preferably 1-4 and most preferably 1-2, carbon atoms, such as methyl, ethyl, propyl, isopropyl, butyl, t-butyl and the like. The term “lower alkyl” is used herein to describe the straight chain alkyl groups described immediately above. Preferably, cycloalkyl groups are monocyclic, bicyclic or tricyclic ring systems of 3-8, more preferably 3-6, ring members per ring, such as cyclopropyl, cyclopentyl, cyclohexyl, adamantyl and the like. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl. A substituted alkyl is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Alkenyl”—alone or in combination means a straight, branched, or cyclic hydrocarbon containing 2-20, preferably 2-17, more preferably 2-10, even more preferably 2-8, most preferably 2-4, carbon atoms and at least one, preferably 1-3, more preferably 1-2, most preferably one, carbon to carbon double bond. In the case of a cycloalkyl group, conjugation of more than one carbon to carbon double bond is not such as to confer aromaticity to the ring. Carbon to carbon double bonds may be either contained within a cycloalkyl portion, with the exception of cyclopropyl, or within a straight chain or branched portion. Examples of alkenyl groups include ethenyl, propenyl, isopropenyl, butenyl, cyclohexenyl, cyclohexenylalkyl and the like. A substituted alkenyl is the straight chain alkenyl, branched alkenyl or cycloalkenyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, carboxy, alkoxycarbonyl, aryloxycarbonyl, heteroaryloxycarbonyl, or the like attached at any available point to produce a stable compound.

“Alkynyl”—alone or in combination means a straight or branched hydrocarbon containing 2-20, preferably 2-17, more preferably 2-10, even more preferably 2-8, most preferably 2-4, carbon atoms containing at least one, preferably one, carbon to carbon triple bond. Examples of alkynyl groups include ethynyl, propynyl, butynyl and the like. A substituted alkynyl refers to the straight chain alkynyl or branched alkenyl defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like attached at any available point to produce a stable compound.

“Alkyl alkenyl” refers to a group —R—CR′═CR′″R″″, where R is lower alkyl, or substituted lower alkyl, R′, R′″, R”” may independently be hydrogen, halogen, lower alkyl, substituted lower alkyl, acyl, aryl, substituted aryl, hetaryl, or substituted hetaryl as defined below.

“Alkyl alkynyl” refers to a groups —RCCR′ where R is lower alkyl or substituted lower alkyl, R′ is hydrogen, lower alkyl, substituted lower alkyl, acyl, aryl, substituted aryl, hetaryl, or substituted hetaryl as defined below.

“Alkoxy” denotes the group —OR, where R is lower alkyl, substituted lower alkyl, acyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

“Alkylthio” or “thioalkoxy” denotes the group —SR, —S(O)ₙ₌₁₋₂—R, where R is lower alkyl, substituted lower alkyl, aryl, substituted aryl, aralkyl or substituted aralkyl as defined herein.

“Acyl” denotes groups —C(O)R, where R is hydrogen, lower alkyl substituted lower alkyl, aryl, substituted aryl and the like as defined herein.

“Aryloxy” denotes groups —OAr, where Ar is an aryl, substituted aryl, heteroaryl, or substituted heteroaryl group as defined herein.

“Amino” or substituted amine denotes the group NRR′, where R and R′ may independently by hydrogen, lower alkyl, substituted lower alkyl, aryl, substituted aryl, hetaryl, or substituted heteroaryl as defined herein, acyl or sulfonyl.

“Amido” denotes the group —C(O)NRR′, where R and R′ may independently by hydrogen, lower alkyl, substituted lower alkyl, aryl, substituted aryl, hetaryl, substituted hetaryl as defined herein.

“Carboxyl” denotes the group —C(O)OR, where R is hydrogen, lower alkyl, substituted lower alkyl, aryl, substituted aryl, hetaryl, and substituted hetaryl as defined herein.

“Aryl”—alone or in combination means phenyl or naphthyl optionally carbocyclic fused with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Substituted aryl” refers to aryl optionally substituted with one or more functional groups, e.g., halogen, lower alkyl, lower alkoxy, alkylthio, acetylene, amino, amido, carboxyl, hydroxyl, aryl, aryloxy, heterocycle, heteroaryl, substituted heteroaryl, nitro, cyano, thiol, sulfamido and the like.

“Heterocycle” refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl or benzo[b]thienyl) and having at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heteroaryl”—alone or in combination means a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group O, S, and N, and optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. Examples of heteroaryl groups are pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl and the like. A substituted heteroaryl contains a substituent attached at an available carbon or nitrogen to produce a stable compound.

“Heterocyclyl”—alone or in combination means a non-aromatic cycloalkyl group having from 5 to 10 atoms in which from 1 to 3 carbon atoms in the ring are replaced by heteroatoms of O, S or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. Examples of heterocyclyl groups are tetrahydrofuranyl, dihydropyridinyl, piperidinyl, pyrrolidinyl, piperazinyl, dihydrobenzofuryl, dihydroindolyl, and the like. A substituted hetercyclyl contains a substituent nitrogen attached at an available carbon or nitrogen to produce a stable compound.

“Substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Aralkyl” refers to the group —R—Ar where Ar is an aryl group and R is lower alkyl or substituted lower alkyl group. Aryl groups can optionally be unsubstituted or substituted with, e.g., halogen, lower alkyl, alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Heteroalkyl” refers to the group —R-Het where Het is a heterocycle group and R is a lower alkyl group. Heteroalkyl groups can optionally be unsubstituted or substituted with e.g., halogen, lower alkyl, lower alkoxy, alkylthio, acetylene, amino, amido, carboxyl, aryl, aryloxy, heterocycle, substituted heterocycle, hetaryl, substituted hetaryl, nitro, cyano, thiol, sulfamido and the like.

“Heteroarylalkyl” refers to the group —R-HetAr where HetAr is an heteroaryl group and R lower alkyl or substituted lower alkyl. Heteroarylalkyl groups can optionally be unsubstituted or substituted with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloalkyl” refers to a divalent cyclic or polycyclic alkyl group containing 3 to 15 carbon atoms.

“Substituted cycloalkyl” refers to a cycloalkyl group comprising one or more substituents with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloheteroalkyl” refers to a cycloalkyl group wherein one or more of the ring carbon atoms is replaced with a heteroatom (e.g., N, O, S or P).

“Substituted cycloheteroalkyl” refers to a cycloheteroalkyl group as herein defined which contains one or more substituents, such as halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloalkyl” denotes the group —R-cycloalkyl where cycloalkyl is a cycloalkyl group and R is a lower alkyl or substituted lower alkyl. Cycloalkyl groups can optionally be unsubstituted or substituted with e.g. halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloheteroalkyl” denotes the group —R-cycloheteroalkyl where R is a lower alkyl or substituted lower alkyl. Cycloheteroalkyl groups can optionally be unsubstituted or substituted with e.g. halogen, lower alkyl, lower alkoxy, alkylthio, amino, amido, carboxyl, acetylene, hydroxyl, aryl, aryloxy, heterocycle, substituted heterocycle, hetaryl, substituted hetaryl, nitro, cyano, thiol, sulfamido and the like.

### Example 15

### Synthesis of the Compounds of Formula I

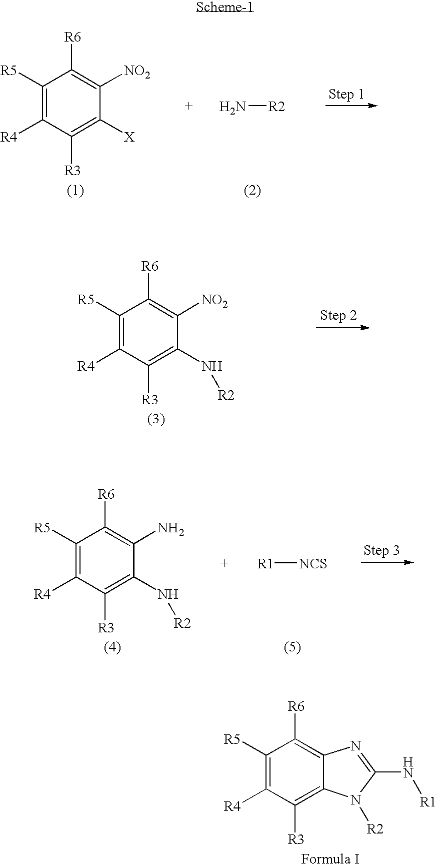

The 2-aminobenzimidazole derivatives, represented by formula I, can be prepared as shown in Scheme-1.

Step-1 Preparation of Formula (3)

The compound of formula (3) is prepared conventionally by reaction of a compound of formula (1), where X═F or Cl (e.g. 2-fluoronitrobenzene), with an amine of formula (2), in an inert solvent (e.g. DMF), in the presence of a base (e.g. K₂CO₃), typically heated near 80° C. for 12-36 hours.

Step-2 Preparation of Formula (4)

The compound of formula (4) is prepared conventionally by reaction of a compound of formula (3) with a reducing agent (e.g. ammonium formate, HCO2NH4), in the presence of a catalyst (e.g. Pd/C), in a suitable solvent (e.g. methanol) at room temperature for several hours. When the reaction is substantially complete, the product of formula (4) is isolated by conventional means; for example, filtration through Celite.

Step-3 Preparation of Formula I

The compound of formula (4) and an isothiocyanate of formula (5) are reacted in the presence of a carbodiimide (e.g. carbonyldiimidazole), in an inert solvent (e.g. DMF). When the reaction is substantially complete, the product of formula I is isolated by conventional means (e.g. reverse phase HPLC). Smith, et. al., (1999) J. Comb. Chem., 1, 368-370; and references therein.

### Example 16

### Synthesis of Compounds of Formula II

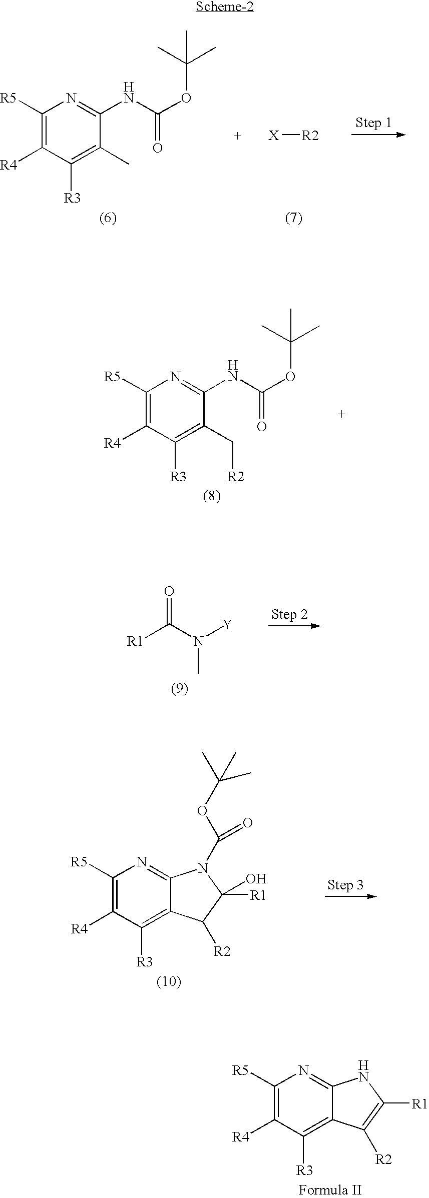

The 7-azaindole derivatives, represented by formula II, can be prepared as shown in Scheme-2.

Step-1 Preparation of Formula (8)

A compound of formula (6) (e.g. 2-tert-butoxycarbonylamino-3-methylpyridine) is reacted with a strong organic base (e.g. n-butyllithium) in an inert solvent (e.g. THF) while cooling. A compound of formula (7) (where X═F, Cl, Br, I, e.g. benzyl bromide), is then added and allowed to react for 30 minutes, at which time the reaction is warmed and quenched with water. The product of formula (8) is isolated by conventional means; for example, aqueous workup, extraction of the product into organic solvent, removal of the solvent under reduced pressure, followed by chromatography of the residue on silica gel.

Step-2 Preparation of Formula (10)

A compound of formula (8) is reacted with a strong organic base (e.g. n-butyllithium) in an inert solvent (e.g. THF) while cooling. Addition of a compound of formula (9), where Y=CH₃ (e.g. DMF) or Y=OCH₃, (i.e. a Weinreb amide, e.g. N-methoxy-N-methylbenzamide), and reaction for approximately an hour at 0° C. results in intermediate of formula (10), which is isolated by conventional means (e.g. aqueous workup) or the reaction mixture is treated as described for Step-3 to directly provide a compound of formula II.

Step-3 Preparation of Formula II

A compound of formula (10) is treated with acid (e.g. 5.5 M HCl) and heated near 45° C. for approximately 1 hour, or the reaction mixture of Step 2 is directly quenched with acid (e.g. 5.5 M HCl) and heated near 40° C. for approximately 2 hours. The product of formula II is isolated by conventional means (e.g. reverse phase HPLC, Kugelrohr distillation, or formation of the tartaric acid salt, followed by filtration and neutralization.) Hands, et. al., (1996) Synthesis, 7, 877; Merour and Joseph, (2001) Curr. Org. Chem. 5, 471-506.

### Example 17

### Synthesis of the Compound of Formula III where Z=O

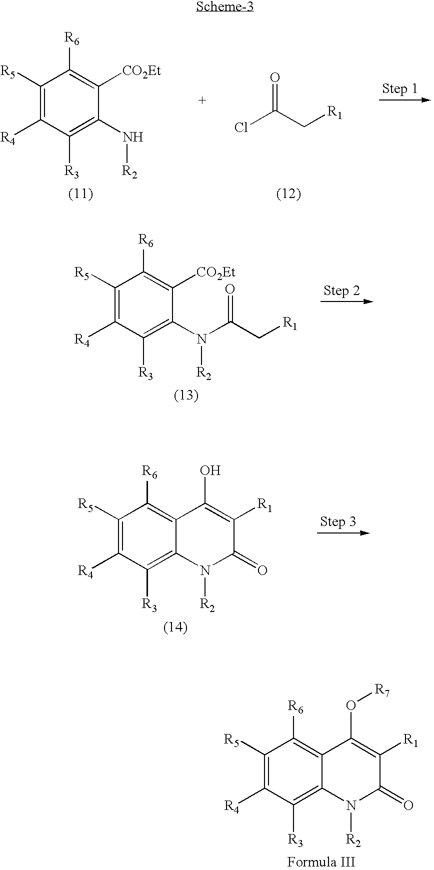

The quinolinone derivatives, represented by Formula III, where Z=0, can be prepared as shown in Scheme-3.

Step-1 Preparation of Formula (13):

The compound of formula (13) can be prepared conventionally by the reaction of a compound (11), for example ethyl 2-aminobenzoate, with an acid chloride of formula (12) in an inert solvent, for example dichloromethane, in presence of a tertiary organic base, for example triethylamine, at room temperature for about 2-24 hours, preferably overnight. When the reaction is substantially complete, the product of formula (13) can be isolated by conventional means, for example aqueous workup, extraction of the product in an organic solvent, removal of the solvent under reduced pressure followed by chromatography of the residue on silica gel.

Step-2 Preparation of Formula (14):

The compound of formula (14) can be prepared from compound of formula (13), by Diekmann cyclization, by stirring with a tertiary organic base or an alkali metal alkoxide, for example potassium t-butoxide, in an inert solvent, for example tetrahydrofuran, at 0° C. to room temperature, preferably room temperature, for about 2-24 hours, preferably 2 hours. When the reaction is substantially complete, product of formula (14) can be isolated by conventional means, for example quenching of the reaction mixture, extraction of the product with organic solvent, for example ethyl acetate, and removal of the solvent under reduced pressure followed by crystallization.

An alternative synthesis of compound of formula (14) starting from 2-nitro-benzoic acid derivative is shown in Scheme-4.

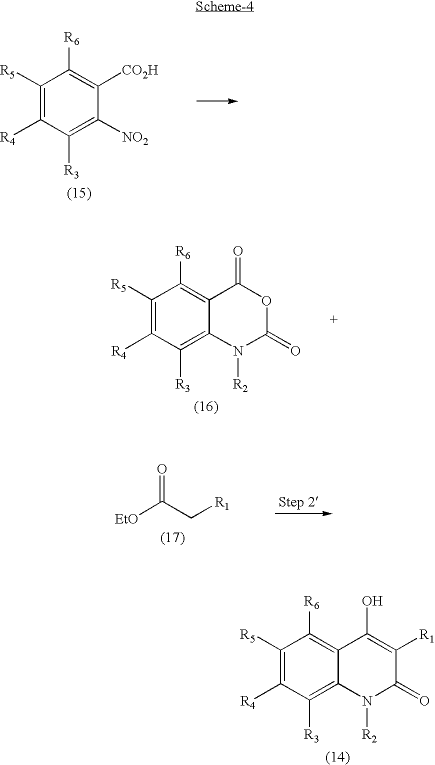

The compound of formula (16) can be reacted with a solution or a suspension of compound of formula (17) and an alkali metal amide, for example lithium diisopropionamide, in an inert solvent, for example THF, −40° C. to room temperature, preferably −40° C., for 2-24 hours, preferably 2 hours. When the reaction is substantially complete, product of formula (14) can be isolated by conventional means, for example quenching of the reaction mixture, extraction of the product with organic solvent, for example ethyl acetate, and removal of the solvent under reduced pressure followed by crystallization.

The compound of formula (16) can be prepared from compound of formula (15) by reduction, for example with hydrazine and ferric chloride in aqueous sodium hydroxide under reflux, cyclization, for example stirring with oxalyl chloride at room temperature, followed by alkylation, for example stirring with R₂-halide and sodium hydride in DMF at room temperature as described in Bioorganic and Medicinal Chemistry Letters 12 (2002) 85-88.

Step-3 Preparation of formula III, where Z=0:

The compound of formula I can be prepared by the reaction of compound of formula (14) with an alkylating agent, for example dimethyl sulfate, in a mixture of solvents, for example methanol and water, under reflux conditions for 2-24 hours, preferably 6 hours. When the reaction is substantially complete, the product of formula III, where Z=0, can be isolated by conventional means.

### Example 18

### Atomic Coordinates for PIM-1 apo protein crystal and co-crystal of PIM-1 with AMPPNP

Solving the structures for PIM-1 crystals and co-crystals provides sets of atomic coordinates for PIM-1 apo protein, and for co-crystals of PIM-1 with binding compounds. Coordinate tables for the apo protein and for PIM-1 co-crystallized with AMP-PNP are provided as Tables 1 and 2 respectively.

In Tables 1 and 2, the various columns have the following content, beginning with the left-most column:

ATOM: Refers to the relevant moeity for the table row.

Atom number: Refers to the arbitrary atom number designation within the coordinate table.

Atom Name: Identifier for the atom present at the particular coordinates.

Chain ID: Chain ID refers to one monomer of the protein in the crystal, e.g., chain “A”, or to other compound present in the crystal, e.g., HOH for water, and L for a ligand or binding compound. Multiple copies of the protein monomers will have different chain Ids.

Residue Number: The amino acid residue number in the chain.

X, Y, Z: Respectively are the X, Y, and Z coordinate values.

Occupancy: Describes the fraction of time the atom is observed in the crystal. For example, occupancy=1 means that the atom is present all the time; occupancy=0.5 indicates that the atom is present in the location 50% of the time.

B-factor: A measure of the thermal motion of the atom.

Element: Identifier for the element.

Atomic coordinates such as those provided allow construction of models and manipulation and analysis of those models.

A number of examples involved in the present invention are described below. In most cases, alternative techniques could also be used. For example, techniques, methods, and other information described in U.S. Pat. No. 5,837,815; U.S. Pat. No. 5,837,524; U.S. Patent Publication 2002/0048782; PCT/US98/02797, WO 98/35056; and McShan et al., Internat. J Oncology 21:197-205 (2002) can be used in the present invention. Such techniques and information include, without limitation, cloning, culturing, purification, assaying, screening, use of modulators, sequence information, and information concerning biological role of PYK2. Each of these references is incorporated by reference herein in its entirety, including drawings.

### Example 19

### Cloning of PYK2 Kinase Domain

Another exemplary identification of scaffolds and use of co-crystals utilized PYK2 kinase. Kinase domain of PYK2 (amino acids 420-691) was amplified by polymerase chain reaction (PCR) using the specific primers 5′-TCCACAGCATATGATTGCCCGTGAAGA TGTGGT-3′ (SEQ ID NO: 5) and 5′-CTCTCGTCGACCTACATGGCAATGTCCTTCTCCA-3′ (SEQ ID NO: 6). The resulting PCR fragment was digested with NdeI and SalI and was ligated into a modified pET15b vector (Novagen) with a cleavable N-terminal hexa-histidine tag (designated pET1S). PYK2 coding sequence has been deposited with GenBank under accession number U33284. A desired PYK2 sequence can be obtained using PCR with a brain (e.g., human brain) cDNA library, such as obtaining kinase domain using the above primers in PCR. The multi-cloning site of the pET15S vector is shown in the following sequence (SEQ ID NO: 7), including the sequence encoding the N-terminal hexa-histadine tag:

[image]

[image]

pET15S vector is derived from pET15b vector (Novagen) for bacterial expression to produce the proteins with N-terminal His6. This vector was modified by replacement of NdeI-BamHI fragment to others to create SalI site and stop codon (TAG). Vector size is 5814 bp. Insert can be put using NdeI-SalI site.

The amino acid and nucleic acid sequences for the PYK2 kinase domain utilized are provided in Table 6 (SEQ ID NO:1 and 3 respectively).

### Example 2

### Expression and Purification of PYK2 Kinase Domain

For protein expression Pyk2 kinase domain was transformed into E. coli strain BL21 (DE3) pLysS and transformants were selected on LB plates containing Kanamycin. Single colonies were grown overnight at 37° C. in 200 ml TB (terrific broth) media. 16×1L of fresh TB media in 2.8L flasks were inoculated with 10 ml of overnight culture and grown with constant shaking at 37° C. Once cultures reached an absorbance of 1.0 at 600 nm, 1 mM isopropyl-β-D-thiogalactopyranoside (IPTG) was added and cultures were allowed to grow for a further 12 hrs at 22° C. with constant shaking. Cells were harvested by centrifugation at 7000×g and pellets were frozen in liquid nitrogen and stored at −80° C. until ready for lysis.

The cell pellet was suspended in lysis buffer containing 0.1M Potassium phosphate buffer pH 8.0, 200 mM NaCl, 10% Glycerol, 2 mm PMSF and EDTA free protease inhibitor cocktail tablets (Roche). Cells were lysed using a microfuidizer processor (Microfuidics Corporation) and insoluble cellular debris was removed using centrifugation at 30,000×g. The cleared supernatant was added to Talon resin (Clonetech) and incubated for 4 hrs at 4° C. with constant rocking. The suspension was loaded onto a column and washed with 20 column volumes of lysis buffer plus 10 mM Imadazole. Protein was eluted step wise with addition of lysis buffer plus 200 mM Imadazole pH7.5 and 1 ml fractions collected. Fractions containing PYK2 were pooled, concentrated and loaded onto a Pharmacia HiLoad 26/60 Superdex 200 sizing column (Pharmacia) pre-equilibrated with 20 mM Tris pH7.5, 150 mM NaCl.

Peak fractions were collected and assayed by SDS-PAGE. Fractions containing PYK2 were pooled and diluted in Tris buffer pH 7.5, until 30 mM NaCl was reached. Diluted protein was further subjected to anion exchange chromatography using a Source 15Q (Pharmacia) sepharose column equilibrated with 20 mM Tris pH7.5. Elution was performed using a linear gradient of sodium chloride (0-500 mM). Eluted protein was treated with 2U thrombin per mg protein to remove N-terminal Histidine tag. Following cleavage Pyk2 was re-applied to Source 15Q (Pharmacia) sepharose column equilibrated with 20 mM Tris pH7.5, and eluted using a linear sodium chloride gradient. Purified protein was concentrated to 100 mg/ml and stored at −80° C. until ready for crystallization screening.

### Example 3

### Crystallization of PYK2 Kinase Domain

Crystallization conditions were initially identified in the Hampton Research (Riverside, Calif.) screening kit₍₁₎. Optimized crystals were grown by vapor diffusion in sitting drop plates with equal volumes of protein solution of 10 mg/ml containing 20 mM Tris-HCl pH 8.0, 150 mM NaCl, 14 mM BME, 1 mM DTT and reservoir solution containing 8% polyethylene glycol (PEG) 8000, 0.2M Sodium Acetate, 0.1M Cacodylate pH 6.5, 20% Glycerol). Blades of crystals grew overnight at 4° C. Microseeding was used to produce larger, single crystals, the largest crystal being around 0.3 mm X 0.05 mm×0.02 mm.

### Example 4

### Diffraction Analysis of PYK2

Synchrotron X-ray data for Pyk2 was collected at beamline 8.3.1 of the Advanced Light Source (ALS, Lawrence Berkeley National Laboratory, Berkeley) on a Quantum 210 charge-coupled device detector (λ=1.10 Å). The mother liquor from the reservoir was used as cryo-protectant for the crystal. Detector distance was 10 mm and exposure time was 10 s per frame. 200 frames were collected with 0.5° oscillation over a wedge of 100°. The quality and resolution limits of the diffraction pattern were considerably improved by annealing the crystal. The crystal was briefly allowed to warm up for 10 seconds by shutting off the Nitrogen cryo stream and refrozen by resuming cooling with the cryo stream. Crystals of PYK2 diffracted to a resolution limit of 1.45 Å with cell dimensions of a=37 Å, b=47 Å, c=81 Å, α=90°, β=92°, γ=90°. The data were processed using Mosflm( ) and scaled and reduced with Scala( ) in CCP4 ( ) in space group P2. The data processing process was driven by the ELVES automation scripts (J. M. Holton, unpublished data). An inspection of the OKO zone indicated that all odd (2n+1) reflections were very weak compared with the even reflections, suggesting the space group to be P2₁.

PYK2 Structure Determination and Refinement

The initial phases for the dataset were obtained by molecular replacement. A homology model of the protein Pyk2 was generated using the LCK kinase structure (PDBID: lqpc) as a template. This model was trimmed by excising all loops before being used in molecular replacement program EPMR ( ), which resulted in a solution with CC=0.372. The molecular replacement solution phases were improved by the program Arp-Warp ( ). The resultant model was further improved by manual model building and extension in O ( ) and refinement with CNX 0 and Refmac5 ( ) in CCP4. The cycle of model building and refinement continued till the model was complete and refinement converged to the R/Rfree of 20.83/26.94%. The geometric analysis of the model was performed by PROCHECK ( ) which indicated the structure to have excellent geometry.

Data collection and refinement statistics for PYK2 kinase domain crystal, and for PYK2 kinase domain/binding compound AMPPNP cocrystal are summarized in the following table:

Data Collection and Refinement Statistics

| | |
|---|---|
| Pyk2 (APO) | Pyk2 + PLX099323 |

Crystal Parameters

| | | |
|---|---|---|
| Space Group | P2₁ | P2₁ |
| Unit Cell (Å) | a = 37.17, b = 46.97, | a = 37.32, b = 46.98, |
| | c = 80.36, =92.63 | c = 81.11, =92.83 |
| Number of molecules/ | 1 | 1 |
| AU | | |
| V_{M} (Å³/Dalton) | 2.4 | 2.4 |
| Solvent content (%) | 48 | 48 |

Data Collection and Processing

| | | |
|---|---|---|
| Resolution (Å) | 1.45 | 1.80 |
| Wavelength (Å) | 1.1 | 1.1 |
| Unique reflections | 47843 | 26149 |
| Redundancy (last shell*) | 2.0 (1.8) | 4.0 (2.9) |
| Completeness (last shell) | 97.5 (88.9) | 99.8 (97.8) |
| (%) | | |
| I/(last shell) | 10.9 (1.3) | 12.0 (2.3) |
| R_{sym} (last shell) | 0.043 (0.487) | 0.063 (0.459) |
| *Last shell (Å) | 1.49-1.45 | 1.85-1.80 |

Refinement

| | | |
|---|---|---|
| R_{work}/R_{free} (%) | 16.93/20.68 | 18.62/22.81 |
| Number of Atoms | 2583 | 2507 |
| Rmsd from ideal | 0.012 (bond distance), | 0.010 (bond distance), |
| geometry | 1.434 (bond angle) | 1.372 (bond angle) |
| SigmaA coordinate error | 0.16 Å | 0.14 Å |
| | (for 5.0-1.45 Å) | (for 5.0-1.80 Å) |
| Average B-factors (Å²) | 19.3 | 20.5 |
| Protein atoms | 16.4 | 19.0 |
| Waters | 37.6 | 34.3 |
| Ligand | — | 44.41 |

The model of Pyk2 contains 273 amino acids (spanning the PYK2 sequence 420-691 with one residue from the cloning vector) and 180 water molecules. The Pyk2 structure adopts the standard kinase fold consisting of an N-terminal β-sheet domain and a C-terminal α-helical domain linked by a 5 residue linker. The linker segment contains the canonical H-bond acceptor/donor residues E503 and Y505 that would normally interact with the adenosine ring of ATP. In the apo structure these residues make H-bonds with water molecules.

Atomic coordinates for PYK2 kinase domain co-crystallized with a binding compound (AMPPNP) are provided in Table 4.

Active Loop Conformation

In many protein kinases, the activation loop, or A-loop, plays an important role in regulating the kinase activity. In active kinases, the A-loops adopt a highly similar conformation characterized by the formation of three small β-sheet moieties: two with the main body of the protein (the beginning of the catalytic or C-loop and the αEF/αF loop, respectively), and one with the substrate peptide. In contrast, the inactive conformation of A-loop differs markedly from protein to protein, albeit having the similar effect of blocking ATP binding, substrate-binding, or both. In comparison with the active insulin receptor (INSR) and IGFR1 kinase domain strutures, the A-loop in the solved Pyk2 structure is clearly in an inactive conformation. The loop is stabilized by a unique set of intra- and inter-loop interactions that differentiate it from all known A-loop structures.

The A-loop in our Pyk2 structure starts to deviate from the standard active conformation at the DFG motif (for comparison, we modeled the active A-loop conformation of Pyk2 based on the IGFR1 structure). The first two residues of the DFG motif (D⁵⁶⁷ and F⁵⁶⁸) have similar orientations as their counterparts in the active A-loop form, with D⁵⁶⁷ interacting with K⁴⁵⁷ (β3) and F⁵⁶⁸ locked in a hydrophobic pocket sandwiched by two residues (I⁴⁷⁷ and M⁴⁷⁸) from aC. However, the third residue in the motif, G⁵⁶⁹, adopts a completely different conformation, resulting in the formation of a hydrogen bond beween G⁵⁶⁷:NH and H⁵⁴⁷:CO. This hydrogen bond forces the A-loop to a different path that precludes it from forming a β-sheet with C-loop. A similar hydrogen bond has also been observed in two other tyrosine kinases: HCK (1qcf) and SRC (1 fink).

There are multiple interactions that help to stabilize the A-loop in its observed conformation. Most of them involve a unique sequence moiety of Pyk2. Among the tyrosine kinases of known structure, Pyk2 contains a unique ED repeat (E⁵⁷⁵-D⁵⁷⁸) in the A-loop. In the Pyk2 structure, E575 is exposed to solvent, whereas D⁵⁷⁶ initiates a tight β-turn. Beside providing the canonical β-turn backbone hydrogen bond between D⁵⁷⁶:CO-Y⁵⁷⁹:NH, the side chain of D⁵⁷⁶ also interacts with D⁵⁷⁸:NH. The β-turn region of A-loop is held to the αEF/αF loop by two side-chain-backbone hydrogen bonds: one between E⁵⁷⁷:CO-R⁶⁰⁰:N^{e} and the other between K⁵⁸¹:NZ-N⁵⁹⁸:CO. The side chain of E⁵⁷⁷ interacts with the end of the activation loop via two hydrogen bonds, one with T⁵⁸⁵ (OG) and the other with R⁵⁸⁶ (NH). The most interesting feature of the Pyk2 A-loop is the salt bridge formed between D⁵⁸⁸ and R⁵⁴⁷ from the C-loop (the distances between the two OD and two NH atoms are 2.9A). Neither of the two tyrosines Y⁵⁷⁹ and y⁵⁸⁰ is phosphorylated in our structure. Y⁵⁷⁹ is exposed to solvent, whereas y⁵⁸⁰ binds to the hydrophobic portions of the E575 and E⁵⁷⁷ side chains.

Because FAK does not have the second ED, the conformation of the A-loop in an inactive FAK is expected to be different.

Implications for Substrate Binding and Autophosphorylation

An important event in the enzymatic activation of FAK/Pyk2 is the autophosphorylation of a tyrosine residue before the catalytic domain (Y402). The phosphorylated Y402 provides the binding site for Src and other related kinases and facilitates Src-dependent phosphorylation of other tyrosine residues on Pyk2 including Y579 and Y580. It is not clear how autophosphorylation could occur before Y579 and Y580 are phosphorylated.

To test whether Y402 can reach the substrate binding site, we modeled the 7 residue peptide D⁴⁰⁰IYAEIPD⁴⁰⁷ containing Y⁴⁰² into the substrate binding site based on the cocrystal structure of IGFR1 kinase domain with its substrate peptide. In our protein construct, the Pyk2 insert starts at 1420. There are four residues (GSHM) N-terminal to 1420 left by the His-tag used, of those only M419 is visible. We then modeled the 11 residues that link D419 to M407. The model shows that, in order to reach the substrate binding site, the N-terminal region has to transverse along the back of aC. The link would also fix the A-loop in the active conformation. This may provide the mechanism that the protein used to autophosphorylate Y402. Once Y402 is phosphorylated, the N-terminus is then released and subject to SH2 binding. The A-loop also becomes flexible and accessible to Src.

Because the residues surrounding the P+1 and P+3 binding pocket are mostly hydrophobic in tyrosine kinases, substrate P+1 and P+3 sites are mostly hydrophobic residues. The residue that might interact with P+2 varies. Acidic and other polar site chains might be preferred because of the nearby residue R⁵⁸⁶. The P−1 site is an acidic residue in INSR and IGFR1. The residue for interacting with P−1 is Arg; this residue is changed to Gly in Pyk2, leaving the space largely hydrophobic. The autophosphorylation site sequence in Pyk2, IYAEIPD, and the sequences of several other known Pyk2 phosphorylation sites fit well the substrate selectivity profile of Pyk2.

### Example 5

### PYK2 Binding Assays

As with PIM-1, binding assays can be performed in a variety of ways, including as described above for PIM-1.

The Pyk2 kinase domain residues 419 to 691 is an active kinase in AlphaScreen. At a concentration of 8 ng/well in 384-well plate, PYK2 shows a Kd of 7.34 uM, which is in general agreement with most protein kinases (Table 7). Inhibition by ATP analogs was tested with Pyk2 at 8 ng/well and ATP at 10 uM. The data is shown in Table 7. The affinity of ATP-g-S and ADP with Pyk2 is at 14 uM. Adenosine and AMP-PCP have little effect on PYK2 in the concentration tested.

### Example 9

### Synthesis of the Compounds of Formula IV

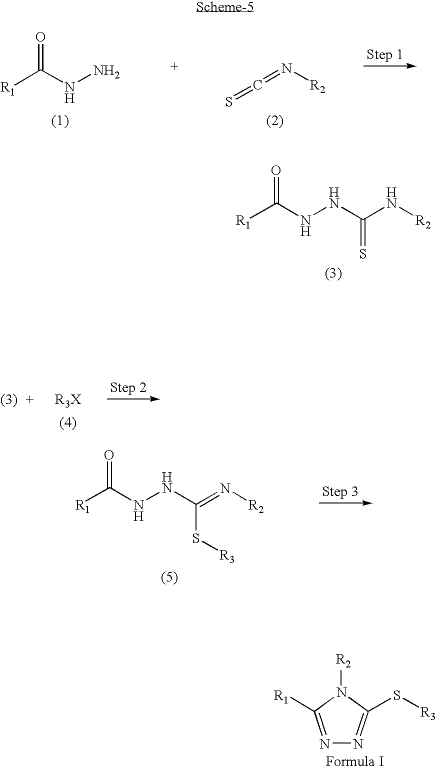

The triazole derivatives, represented by Formula IV, can be prepared as shown in Scheme-5.

Step-1 Preparation of Formula (3)

The compound of formula (3) is prepared conventionally by reaction of a compound of formula (1), where R₁=alkyl, aryl, heteroaryl (e.g. m-toluic hydrazide), with an isothiocyanate of formula (2), in a basic solvent (e.g. pyridine), typically heated near 65° C. for 2-6 hours.

Step-2 Preparation of Formula (5)

The compound of formula (5) is prepared conventionally by reaction of a compound of formula (3) with an alkylating agent of formula (4)(e.g. methyl iodide), in an inert solvent (e.g. THF) at room temperature for 24-48 hours.

Step-3 Preparation of Formula I

The compound of Formula IV is prepared by dissolving a compound of formula (5) in POCl3 and heated near 80° C. for 8-12 hours. When the reaction is substantially complete, the product of Formula I is isolated by conventional means (e.g. reverse phase HPLC). Smith, et. al., J. Comb. Chem., 1999, 1, 368-370; and references therein.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, using other binding assays and other modifications to molecular scaffold compounds are all within the scope of the present invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

TABLE 1

| | | |
|---|---|---|
| HEADER | ---- | XX-XXX-XX xxxx |

| | |
|---|---|
| COMPND | --- |

| REMARK | 3 | | |
|---|---|---|---|
| REMARK | 3 | REFINEMENT. | |
| REMARK | 3 | PROGRAM | REFMAC 5.1.19 |
| REMARK | 3 | AUTHORS | MURSHUDOV,VAGIN,DODSON |
| REMARK | 3 | | |

| | | | |
|---|---|---|---|
| REMARK | 3 | REFINEMENT TARGET | MAXIMUM LIKELIHOOD |
| REMARK | 3 | | |

| | | | |
|---|---|---|---|
| REMARK | 3 | DATA USED IN REFINEMENT. | |
| REMARK | 3 | RESOLUTION RANGE HIGH (ANGSTROMS) | 2.00 |
| REMARK | 3 | RESOLUTION RANGE LOW (ANGSTROMS) | 84.52 |
| REMARK | 3 | DATA CUTOFF (SIGMA(F)) | NONE |
| REMARK | 3 | COMPLETENESS FOR RANGE (%) | 99.27 |
| REMARK | 3 | NUMBER OF REFLECTIONS | 28693 |
| REMARK | 3 | | |
| REMARK | 3 | FIT TO DATA USED IN REFINEMENT. | |
| REMARK | 3 | CROSS-VALIDATION METHOD | THROUGHOUT |
| REMARK | 3 | FREE R VALUE TEST SET SELECTION | RANDOM |
| REMARK | 3 | R VALUE (WORKING + TEST SET) | 0.22119 |
| REMARK | 3 | R VALUE (WORKING SET) | 0.22012 |
| REMARK | 3 | FREE R VALUE | 0.24194 |
| REMARK | 3 | FREE R VALUE TEST SET SIZE (%) | 5.0 |
| REMARK | 3 | FREE R VALUE TEST SET COUNT | 1498 |
| REMARK | 3 | | |
| REMARK | 3 | FIT IN THE HIGHEST RESOLUTION BIN. | |
| REMARK | 3 | TOTAL NUMBER OF BINS USED | 20 |
| REMARK | 3 | BIN RESOLUTION RANGE HIGH | 2.000 |
| REMARK | 3 | BIN RESOLUTION RANGE LOW | 2.052 |
| REMARK | 3 | REFLECTION IN BIN (WORKING SET) | 2096 |
| REMARK | 3 | BIN R VALUE (WORKING SET) | 0.344 |
| REMARK | 3 | BIN FREE R VALUE SET COUNT | 102 |
| REMARK | 3 | BIN FREE R VALUE | 0.359 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | 2382 |
| REMARK | 3 | | |
| REMARK | 3 | B VALUES. | |

| | | | |
|---|---|---|---|
| REMARK | 3 | FROM WILSON PLOT (A**2) | NULL |
| REMARK | 3 | MEAN B VALUE (OVERALL, A**2) | 49.236 |

| | | |
|---|---|---|
| REMARK | 3 | OVERALL ANISOTROPIC B VALUE. |

| | | | |
|---|---|---|---|
| REMARK | 3 | B11 (A**2) | 1.32 |
| REMARK | 3 | B22 (A**2) | 1.32 |
| REMARK | 3 | B33 (A**2) | −1.99 |
| REMARK | 3 | B12 (A**2) | 0.66 |
| REMARK | 3 | B13 (A**2) | 0.00 |
| REMARK | 3 | B23 (A**2) | 0.00 |
| REMARK | 3 | | |

| | | | |
|---|---|---|---|
| REMARK | 3 | ESTIMATED OVERALL COORDINATE ERROR. | |
| REMARK | 3 | ESU BASED ON R VALUE (A) | 0.158 |
| REMARK | 3 | ESU BASED ON FREE R VALUE (A) | 0.142 |
| REMARK | 3 | ESU BASED ON MAXIMUM LIKELIHOOD (A) | 0.127 |
| REMARK | 3 | ESU FOR B VALUES BASED ON MAXIMUM LIKELIHOOD (A**2) | 4.758 |
| REMARK | 3 | | |

| | | | |
|---|---|---|---|
| REMARK | 3 | CORRELATION COEFFICIENTS. | |
| REMARK | 3 | CORRELATION COEFFICIENT FO-FC | 0.954 |
| REMARK | 3 | CORRELATION COEFFICIENT FO-FC FREE | 0.947 |
| REMARK | 3 | | |

| | | | | | |
|---|---|---|---|---|---|
| REMARK | 3 | RMS DEVIATIONS FROM IDEAL VALUES | COUNT | RMS | WEIGHT |
| REMARK | 3 | BOND LENGTHS REFINED ATOMS (A) | 2296 | 0.011 | 0.021 |
| REMARK | 3 | BOND ANGLES REFINED ATOMS (DEGREES) | 3114 | 1.088 | 1.945 |
| REMARK | 3 | TORSION ANGLES, PERIOD 1 (DEGREES) | 273 | 3.838 | 5.000 |
| REMARK | 3 | CHIRAL-CENTER RESTRAINTS (A**3) | 332 | 0.081 | 0.200 |
| REMARK | 3 | GENERAL PLANES REFINED ATOMS (A) | 1784 | 0.004 | 0.020 |
| REMARK | 3 | NON-BONDED CONTACTS REFINED ATOMS (A) | 1094 | 0.215 | 0.200 |
| REMARK | 3 | H-BOND (X...Y) REFINED ATOMS (A) | 138 | 0.121 | 0.200 |
| REMARK | 3 | SYMMETRY VDW REFINED ATOMS (A) | 60 | 0.282 | 0.200 |
| REMARK | 3 | SYMMETRY H-BOND REFINED ATOMS (A) | 19 | 0.247 | 0.200 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2) | 1365 | 1.058 | 1.500 |
| REMARK | 3 | MAIN-CHAIN ANGLE REFINED ATOMS (A**2) | 2212 | 2.010 | 2.000 |
| REMARK | 3 | SIDE-CHAIN BOND REFINED ATOMS (A**2) | 931 | 2.240 | 3.000 |
| REMARK | 3 | SIDE-CHAIN ANGLE REFINED ATOMS (A**2) | 902 | 3.766 | 4.500 |
| REMARK | 3 | | | | |

| | | | |
|---|---|---|---|
| REMARK | 3 | NCS RESTRAINTS STATISTICS | |
| REMARK | 3 | NUMBER OF NCS GROUPS | NULL |
| REMARK | 3 | | |
| REMARK | 3 | | |
| REMARK | 3 | TLS DETAILS | |
| REMARK | 3 | NUMBER OF TLS GROUPS | NULL |
| REMARK | 3 | | |
| REMARK | 3 | | |
| REMARK | 3 | BULK SOLVENT MODELLING. | |

| | | | |
|---|---|---|---|
| REMARK | 3 | METHOD USED | BABINET MODEL WITH MASK |

| REMARK | 3 | PARAMETERS FOR MASK CALCULATION | |
|---|---|---|---|
| REMARK | 3 | VDW PROBE RADIUS | 1.40 |
| REMARK | 3 | ION PROBE RADIUS | 0.80 |
| REMARK | 3 | SHRINKAGE RADIUS | 0.80 |
| REMARK | 3 | | |

| | | | |
|---|---|---|---|
| REMARK | 3 | OTHER REFINEMENT REMARKS | NULL |
| REMARK | 3 | | |

| | |
|---|---|
| CISPEP | 1 GLU A 124 PRO A 125 0.00 |
| CRYST1 | 99.210 99.210 80.285 90.00 90.00 120.00 P 65 |

| | |
|---|---|
| SCALE1 | 0.010080 0.005819 0.000000 0.00000 |
| SCALE2 | 0.000000 0.011639 0.000000 0.00000 |
| SCALE3 | 0.000000 0.000000 0.012456 0.00000 |

| | | | | | | | | | | | |
|---|---|---|---|---|---|---|---|---|---|---|---|
| ATOM | 1 | N | PRO | A | 33 | 9.285 | 100.137 | −4.493 | 1.00 | 93.84 | N |
| ATOM | 2 | CA | PRO | A | 33 | 8.922 | 99.154 | −3.430 | 1.00 | 93.59 | C |
| ATOM | 3 | CB | PRO | A | 33 | 9.624 | 97.864 | −3.896 | 1.00 | 93.79 | C |
| ATOM | 4 | CG | PRO | A | 33 | 10.732 | 98.328 | −4.833 | 1.00 | 93.76 | C |
| ATOM | 5 | CD | PRO | A | 33 | 10.201 | 99.562 | −5.499 | 1.00 | 93.83 | C |
| ATOM | 6 | C | PRO | A | 33 | 9.413 | 99.588 | −2.038 | 1.00 | 93.22 | C |
| ATOM | 7 | O | PRO | A | 33 | 8.647 | 100.212 | −1.288 | 1.00 | 93.33 | O |
| ATOM | 8 | N | LEU | A | 34 | 10.667 | 99.251 | −1.716 | 1.00 | 92.55 | N |
| ATOM | 9 | CA | LEU | A | 34 | 11.325 | 99.616 | −0.457 | 1.00 | 91.82 | C |
| ATOM | 10 | CB | LEU | A | 34 | 11.402 | 101.150 | −0.303 | 1.00 | 92.11 | C |
| ATOM | 11 | CG | LEU | A | 34 | 12.362 | 101.709 | 0.756 | 1.00 | 92.47 | C |
| ATOM | 12 | CD1 | LEU | A | 34 | 13.829 | 101.513 | 0.349 | 1.00 | 92.34 | C |
| ATOM | 13 | CD2 | LEU | A | 34 | 12.044 | 103.183 | 1.024 | 1.00 | 93.01 | C |
| ATOM | 14 | C | LEU | A | 34 | 10.758 | 98.941 | 0.808 | 1.00 | 90.98 | C |
| ATOM | 15 | O | LEU | A | 34 | 11.164 | 97.828 | 1.157 | 1.00 | 91.10 | O |
| ATOM | 16 | N | GLU | A | 35 | 9.837 | 99.614 | 1.498 | 1.00 | 89.80 | N |
| ATOM | 17 | CA | GLU | A | 35 | 9.346 | 99.114 | 2.780 | 1.00 | 88.50 | C |
| ATOM | 18 | CB | GLU | A | 35 | 10.297 | 99.526 | 3.901 | 1.00 | 88.76 | C |
| ATOM | 19 | CG | GLU | A | 35 | 10.444 | 101.039 | 4.047 | 1.00 | 89.07 | C |
| ATOM | 20 | CD | GLU | A | 35 | 11.208 | 101.436 | 5.292 | 1.00 | 89.82 | C |
| ATOM | 21 | OE1 | GLU | A | 35 | 10.603 | 101.403 | 6.400 | 1.00 | 90.45 | O |
| ATOM | 22 | OE2 | GLU | A | 35 | 12.411 | 101.780 | 5.162 | 1.00 | 89.60 | O |
| ATOM | 23 | C | GLU | A | 35 | 7.963 | 99.672 | 3.060 | 1.00 | 87.48 | C |
| ATOM | 24 | O | GLU | A | 35 | 7.220 | 99.114 | 3.875 | 1.00 | 87.62 | O |
| ATOM | 25 | N | SER | A | 36 | 7.640 | 100.781 | 2.382 | 1.00 | 85.74 | N |
| ATOM | 26 | CA | SER | A | 36 | 6.316 | 101.427 | 2.424 | 1.00 | 83.76 | C |
| ATOM | 27 | CB | SER | A | 36 | 6.258 | 102.576 | 1.402 | 1.00 | 84.10 | C |
| ATOM | 28 | OG | SER | A | 36 | 7.465 | 103.332 | 1.399 | 1.00 | 84.47 | O |
| ATOM | 29 | C | SER | A | 36 | 5.170 | 100.444 | 2.150 | 1.00 | 81.91 | C |
| ATOM | 30 | O | SER | A | 36 | 3.997 | 100.755 | 2.389 | 1.00 | 81.51 | O |
| ATOM | 31 | N | GLN | A | 37 | 5.535 | 99.262 | 1.651 | 1.00 | 79.60 | N |
| ATOM | 32 | CA | GLN | A | 37 | 4.600 | 98.179 | 1.363 | 1.00 | 77.25 | C |
| ATOM | 33 | CB | GLN | A | 37 | 5.316 | 97.058 | 0.614 | 1.00 | 77.48 | C |
| ATOM | 34 | CG | GLN | A | 37 | 6.195 | 97.509 | −0.554 | 1.00 | 77.20 | C |
| ATOM | 35 | CD | GLN | A | 37 | 6.645 | 96.330 | −1.414 | 1.00 | 77.20 | C |
| ATOM | 36 | OE1 | GLN | A | 37 | 5.827 | 95.483 | −1.799 | 1.00 | 77.03 | O |
| ATOM | 37 | NE2 | GLN | A | 37 | 7.942 | 96.268 | −1.709 | 1.00 | 76.81 | N |
| ATOM | 38 | C | GLN | A | 37 | 3.970 | 97.604 | 2.623 | 1.00 | 75.49 | C |
| ATOM | 39 | O | GLN | A | 37 | 2.879 | 97.043 | 2.567 | 1.00 | 75.51 | O |
| ATOM | 40 | N | TYR | A | 38 | 4.655 | 97.747 | 3.756 | 1.00 | 73.43 | N |
| ATOM | 41 | CA | TYR | A | 38 | 4.208 | 97.129 | 5.004 | 1.00 | 71.44 | C |
| ATOM | 42 | CB | TYR | A | 38 | 5.100 | 95.931 | 5.373 | 1.00 | 70.49 | C |
| ATOM | 43 | CG | TYR | A | 38 | 5.227 | 94.919 | 4.255 | 1.00 | 67.67 | C |
| ATOM | 44 | CD1 | TYR | A | 38 | 4.258 | 93.929 | 4.067 | 1.00 | 65.14 | C |
| ATOM | 45 | CE1 | TYR | A | 38 | 4.361 | 93.019 | 3.032 | 1.00 | 63.31 | C |
| ATOM | 46 | CZ | TYR | A | 38 | 5.446 | 93.087 | 2.177 | 1.00 | 62.94 | C |
| ATOM | 47 | OH | TYR | A | 38 | 5.568 | 92.191 | 1.151 | 1.00 | 64.24 | O |
| ATOM | 48 | CE2 | TYR | A | 38 | 6.417 | 94.054 | 2.339 | 1.00 | 63.82 | C |
| ATOM | 49 | CD2 | TYR | A | 38 | 6.304 | 94.967 | 3.371 | 1.00 | 65.13 | C |
| ATOM | 50 | C | TYR | A | 38 | 4.125 | 98.099 | 6.169 | 1.00 | 71.00 | C |
| ATOM | 51 | O | TYR | A | 38 | 5.021 | 98.914 | 6.385 | 1.00 | 70.68 | O |
| ATOM | 52 | N | GLN | A | 39 | 3.026 | 97.986 | 6.913 | 1.00 | 70.43 | N |
| ATOM | 53 | CA | GLN | A | 39 | 2.797 | 98.756 | 8.124 | 1.00 | 69.86 | C |
| ATOM | 54 | CB | GLN | A | 39 | 1.298 | 99.021 | 8.279 | 1.00 | 70.46 | C |
| ATOM | 55 | CG | GLN | A | 39 | 0.934 | 100.007 | 9.385 | 1.00 | 73.80 | C |
| ATOM | 56 | CD | GLN | A | 39 | 0.378 | 99.319 | 10.635 | 1.00 | 77.97 | C |
| ATOM | 57 | OE1 | GLN | A | 39 | −0.750 | 98.794 | 10.625 | 1.00 | 79.52 | O |
| ATOM | 58 | NE2 | GLN | A | 39 | 1.161 | 99.330 | 11.717 | 1.00 | 78.94 | N |
| ATOM | 59 | C | GLN | A | 39 | 3.333 | 97.967 | 9.322 | 1.00 | 68.49 | C |
| ATOM | 60 | O | GLN | A | 39 | 2.704 | 97.003 | 9.777 | 1.00 | 68.58 | O |
| ATOM | 61 | N | VAL | A | 40 | 4.491 | 98.390 | 9.834 | 1.00 | 66.87 | N |
| ATOM | 62 | CA | VAL | A | 40 | 5.141 | 97.688 | 10.940 | 1.00 | 65.53 | C |
| ATOM | 63 | CB | VAL | A | 40 | 6.600 | 98.137 | 11.138 | 1.00 | 65.20 | C |
| ATOM | 64 | CG1 | VAL | A | 40 | 7.310 | 97.201 | 12.100 | 1.00 | 64.63 | C |
| ATOM | 65 | CG2 | VAL | A | 40 | 7.336 | 98.174 | 9.804 | 1.00 | 65.16 | C |
| ATOM | 66 | C | VAL | A | 40 | 4.376 | 97.837 | 12.255 | 1.00 | 64.96 | C |
| ATOM | 67 | O | VAL | A | 40 | 3.833 | 98.893 | 12.547 | 1.00 | 65.27 | O |
| ATOM | 68 | N | GLY | A | 41 | 4.339 | 96.766 | 13.042 | 1.00 | 64.02 | N |
| ATOM | 69 | CA | GLY | A | 41 | 3.640 | 96.764 | 14.310 | 1.00 | 62.22 | C |
| ATOM | 70 | C | GLY | A | 41 | 4.545 | 96.341 | 15.451 | 1.00 | 61.31 | C |
| ATOM | 71 | O | GLY | A | 41 | 5.747 | 96.572 | 15.406 | 1.00 | 60.92 | O |
| ATOM | 72 | N | PRO | A | 42 | 3.966 | 95.725 | 16.478 | 1.00 | 60.62 | N |
| ATOM | 73 | CA | PRO | A | 42 | 4.723 | 95.313 | 17.666 | 1.00 | 60.91 | C |
| ATOM | 74 | CB | PRO | A | 42 | 3.636 | 94.755 | 18.602 | 1.00 | 60.81 | C |
| ATOM | 75 | CG | PRO | A | 42 | 2.347 | 95.332 | 18.089 | 1.00 | 60.97 | C |
| ATOM | 76 | CD | PRO | A | 42 | 2.529 | 95.401 | 16.599 | 1.00 | 60.64 | C |
| ATOM | 77 | C | PRO | A | 42 | 5.759 | 94.235 | 17.385 | 1.00 | 60.96 | C |
| ATOM | 78 | O | PRO | A | 42 | 5.626 | 93.478 | 16.424 | 1.00 | 60.93 | O |
| ATOM | 79 | N | LEU | A | 43 | 6.783 | 94.180 | 18.226 | 1.00 | 61.11 | N |
| ATOM | 80 | CA | LEU | A | 43 | 7.737 | 93.084 | 18.200 | 1.00 | 61.79 | C |
| ATOM | 81 | CB | LEU | A | 43 | 8.924 | 93.411 | 19.110 | 1.00 | 61.59 | C |
| ATOM | 82 | CG | LEU | A | 43 | 10.162 | 92.511 | 19.107 | 1.00 | 62.19 | C |
| ATOM | 83 | CD1 | LEU | A | 43 | 11.000 | 92.704 | 17.848 | 1.00 | 61.21 | C |
| ATOM | 84 | CD2 | LEU | A | 43 | 11.003 | 92.782 | 20.344 | 1.00 | 62.67 | C |
| ATOM | 85 | C | LEU | A | 43 | 7.027 | 91.795 | 18.643 | 1.00 | 62.48 | C |
| ATOM | 86 | O | LEU | A | 43 | 6.143 | 91.824 | 19.511 | 1.00 | 62.19 | O |
| ATOM | 87 | N | LEU | A | 44 | 7.396 | 90.671 | 18.030 | 1.00 | 63.26 | N |
| ATOM | 88 | CA | LEU | A | 44 | 6.811 | 89.378 | 18.387 | 1.00 | 63.89 | C |
| ATOM | 89 | CB | LEU | A | 44 | 6.257 | 88.663 | 17.154 | 1.00 | 63.70 | C |
| ATOM | 90 | CG | LEU | A | 44 | 5.135 | 89.362 | 16.379 | 1.00 | 63.05 | C |
| ATOM | 91 | CD1 | LEU | A | 44 | 4.801 | 88.562 | 15.131 | 1.00 | 62.30 | C |
| ATOM | 92 | CD2 | LEU | A | 44 | 3.894 | 89.539 | 17.241 | 1.00 | 62.27 | C |
| ATOM | 93 | C | LEU | A | 44 | 7.791 | 88.474 | 19.110 | 1.00 | 64.82 | C |
| ATOM | 94 | O | LEU | A | 44 | 7.386 | 87.669 | 19.951 | 1.00 | 65.08 | O |
| ATOM | 95 | N | GLY | A | 45 | 9.071 | 88.602 | 18.784 | 1.00 | 66.08 | N |
| ATOM | 96 | CA | GLY | A | 45 | 10.088 | 87.734 | 19.357 | 1.00 | 68.09 | C |
| ATOM | 97 | C | GLY | A | 45 | 11.517 | 88.122 | 19.027 | 1.00 | 69.52 | C |
| ATOM | 98 | O | GLY | A | 45 | 11.763 | 88.937 | 18.124 | 1.00 | 69.05 | O |
| ATOM | 99 | N | SER | A | 46 | 12.448 | 87.517 | 19.774 | 1.00 | 71.08 | N |
| ATOM | 100 | CA | SER | A | 46 | 13.891 | 87.764 | 19.662 | 1.00 | 72.58 | C |
| ATOM | 101 | CB | SER | A | 46 | 14.311 | 88.922 | 20.588 | 1.00 | 72.92 | C |
| ATOM | 102 | OG | SER | A | 46 | 15.655 | 89.327 | 20.364 | 1.00 | 74.04 | O |
| ATOM | 103 | C | SER | A | 46 | 14.688 | 86.513 | 20.027 | 1.00 | 73.06 | C |
| ATOM | 104 | O | SER | A | 46 | 14.265 | 85.720 | 20.875 | 1.00 | 73.26 | O |
| ATOM | 105 | N | GLY | A | 47 | 15.849 | 86.349 | 19.394 | 1.00 | 73.67 | N |
| ATOM | 106 | CA | GLY | A | 47 | 16.733 | 85.234 | 19.707 | 1.00 | 74.04 | C |
| ATOM | 107 | C | GLY | A | 47 | 17.739 | 84.965 | 18.608 | 1.00 | 74.12 | C |
| ATOM | 108 | O | GLY | A | 47 | 18.133 | 85.889 | 17.883 | 1.00 | 74.48 | O |
| ATOM | 109 | N | GLY | A | 48 | 18.150 | 83.698 | 18.490 | 1.00 | 73.84 | N |
| ATOM | 110 | CA | GLY | A | 48 | 19.109 | 83.257 | 17.478 | 1.00 | 73.16 | C |
| ATOM | 111 | C | GLY | A | 48 | 18.602 | 83.392 | 16.048 | 1.00 | 72.45 | C |
| ATOM | 112 | O | GLY | A | 48 | 19.391 | 83.374 | 15.093 | 1.00 | 72.37 | O |
| ATOM | 113 | N | PHE | A | 49 | 17.282 | 83.531 | 15.911 | 1.00 | 71.52 | N |
| ATOM | 114 | CA | PHE | A | 49 | 16.647 | 83.755 | 14.612 | 1.00 | 70.43 | C |
| ATOM | 115 | CB | PHE | A | 49 | 15.215 | 83.187 | 14.590 | 1.00 | 70.83 | C |
| ATOM | 116 | CG | PHE | A | 49 | 14.301 | 83.752 | 15.661 | 1.00 | 73.19 | C |
| ATOM | 117 | CD1 | PHE | A | 49 | 13.584 | 84.933 | 15.439 | 1.00 | 74.32 | C |
| ATOM | 118 | CE1 | PHE | A | 49 | 12.738 | 85.453 | 16.419 | 1.00 | 75.71 | C |
| ATOM | 119 | CZ | PHE | A | 49 | 12.587 | 84.787 | 17.638 | 1.00 | 75.96 | C |
| ATOM | 120 | CE2 | PHE | A | 49 | 13.290 | 83.605 | 17.874 | 1.00 | 75.42 | C |
| ATOM | 121 | CD2 | PHE | A | 49 | 14.139 | 83.090 | 16.883 | 1.00 | 74.82 | C |
| ATOM | 122 | C | PHE | A | 49 | 16.696 | 85.231 | 14.157 | 1.00 | 68.55 | C |
| ATOM | 123 | O | PHE | A | 49 | 16.785 | 85.509 | 12.963 | 1.00 | 69.15 | O |
| ATOM | 124 | N | GLY | A | 50 | 16.663 | 86.164 | 15.106 | 1.00 | 66.20 | N |
| ATOM | 125 | CA | GLY | A | 50 | 16.625 | 87.588 | 14.795 | 1.00 | 62.55 | C |
| ATOM | 126 | C | GLY | A | 50 | 15.562 | 88.351 | 15.578 | 1.00 | 59.75 | C |
| ATOM | 127 | O | GLY | A | 50 | 15.316 | 88.056 | 16.754 | 1.00 | 59.86 | O |
| ATOM | 128 | N | SER | A | 51 | 14.945 | 89.332 | 14.916 | 1.00 | 56.20 | N |
| ATOM | 129 | CA | SER | A | 51 | 13.866 | 90.148 | 15.480 | 1.00 | 51.75 | C |
| ATOM | 130 | CB | SER | A | 51 | 14.300 | 91.614 | 15.587 | 1.00 | 51.18 | C |
| ATOM | 131 | OG | SER | A | 51 | 15.454 | 91.750 | 16.401 | 1.00 | 48.30 | O |
| ATOM | 132 | C | SER | A | 51 | 12.699 | 90.076 | 14.537 | 1.00 | 49.79 | C |
| ATOM | 133 | O | SER | A | 51 | 12.848 | 90.341 | 13.344 | 1.00 | 48.22 | O |
| ATOM | 134 | N | VAL | A | 52 | 11.538 | 89.724 | 15.064 | 1.00 | 47.77 | N |
| ATOM | 135 | CA | VAL | A | 52 | 10.345 | 89.551 | 14.243 | 1.00 | 46.96 | C |
| ATOM | 136 | CB | VAL | A | 52 | 9.795 | 88.091 | 14.312 | 1.00 | 46.48 | C |
| ATOM | 137 | CG1 | VAL | A | 52 | 8.570 | 87.924 | 13.397 | 1.00 | 45.18 | C |
| ATOM | 138 | CG2 | VAL | A | 52 | 10.873 | 87.082 | 13.955 | 1.00 | 45.83 | C |
| ATOM | 139 | C | VAL | A | 52 | 9.265 | 90.515 | 14.701 | 1.00 | 47.44 | C |
| ATOM | 140 | O | VAL | A | 52 | 8.874 | 90.493 | 15.869 | 1.00 | 48.09 | O |
| ATOM | 141 | N | TYR | A | 53 | 8.784 | 91.346 | 13.779 | 1.00 | 47.68 | N |
| ATOM | 142 | CA | TYR | A | 53 | 7.723 | 92.315 | 14.055 | 1.00 | 48.21 | C |
| ATOM | 143 | CB | TYR | A | 53 | 8.102 | 93.711 | 13.532 | 1.00 | 47.13 | C |
| ATOM | 144 | CG | TYR | A | 53 | 9.290 | 94.335 | 14.223 | 1.00 | 46.28 | C |
| ATOM | 145 | CD1 | TYR | A | 53 | 10.593 | 93.949 | 13.897 | 1.00 | 43.98 | C |
| ATOM | 146 | CE1 | TYR | A | 53 | 11.689 | 94.495 | 14.532 | 1.00 | 42.59 | C |
| ATOM | 147 | CZ | TYR | A | 53 | 11.498 | 95.475 | 15.521 | 1.00 | 43.72 | C |
| ATOM | 148 | OH | TYR | A | 53 | 12.598 | 96.012 | 16.159 | 1.00 | 43.55 | O |
| ATOM | 149 | CE2 | TYR | A | 53 | 10.226 | 95.884 | 15.864 | 1.00 | 44.06 | C |
| ATOM | 150 | CD2 | TYR | A | 53 | 9.117 | 95.305 | 15.221 | 1.00 | 45.68 | C |
| ATOM | 151 | C | TYR | A | 53 | 6.436 | 91.886 | 13.363 | 1.00 | 49.25 | C |
| ATOM | 152 | O | TYR | A | 53 | 6.466 | 91.335 | 12.258 | 1.00 | 48.07 | O |
| ATOM | 153 | N | SER | A | 54 | 5.306 | 92.162 | 14.008 | 1.00 | 50.84 | N |
| ATOM | 154 | CA | SER | A | 54 | 3.996 | 91.959 | 13.398 | 1.00 | 53.09 | C |
| ATOM | 155 | CB | SER | A | 54 | 2.889 | 92.092 | 14.445 | 1.00 | 53.24 | C |
| ATOM | 156 | OG | SER | A | 54 | 1.609 | 91.939 | 13.854 | 1.00 | 55.73 | O |
| ATOM | 157 | C | SER | A | 54 | 3.826 | 93.019 | 12.342 | 1.00 | 54.41 | C |
| ATOM | 158 | O | SER | A | 54 | 4.303 | 94.129 | 12.510 | 1.00 | 55.46 | O |
| ATOM | 159 | N | GLY | A | 55 | 3.151 | 92.691 | 11.248 | 1.00 | 56.17 | N |
| ATOM | 160 | CA | GLY | A | 55 | 3.043 | 93.625 | 10.143 | 1.00 | 58.09 | C |
| ATOM | 161 | C | GLY | A | 55 | 1.800 | 93.391 | 9.327 | 1.00 | 60.22 | C |
| ATOM | 162 | O | GLY | A | 55 | 1.164 | 92.343 | 9.433 | 1.00 | 60.35 | O |
| ATOM | 163 | N | ILE | A | 56 | 1.457 | 94.381 | 8.513 | 1.00 | 62.12 | N |
| ATOM | 164 | CA | ILE | A | 56 | 0.307 | 94.305 | 7.635 | 1.00 | 64.34 | C |
| ATOM | 165 | CB | ILE | A | 56 | −0.847 | 95.188 | 8.169 | 1.00 | 64.42 | C |
| ATOM | 166 | CG1 | ILE | A | 56 | −1.391 | 94.639 | 9.500 | 1.00 | 65.47 | C |
| ATOM | 167 | CD1 | ILE | A | 56 | −2.240 | 95.670 | 10.281 | 1.00 | 66.61 | C |
| ATOM | 168 | CG2 | ILE | A | 56 | −1.969 | 95.273 | 7.149 | 1.00 | 65.46 | C |
| ATOM | 169 | C | ILE | A | 56 | 0.759 | 94.780 | 6.267 | 1.00 | 65.56 | C |
| ATOM | 170 | O | ILE | A | 56 | 1.422 | 95.805 | 6.155 | 1.00 | 65.96 | O |
| ATOM | 171 | N | ARG | A | 57 | 0.419 | 94.017 | 5.233 | 1.00 | 67.26 | N |
| ATOM | 172 | CA | ARG | A | 57 | 0.731 | 94.386 | 3.858 | 1.00 | 68.96 | C |
| ATOM | 173 | CB | ARG | A | 57 | 0.628 | 93.161 | 2.946 | 1.00 | 68.74 | C |
| ATOM | 174 | CG | ARG | A | 57 | 1.139 | 93.361 | 1.520 | 1.00 | 68.49 | C |
| ATOM | 175 | CD | ARG | A | 57 | 0.433 | 92.424 | 0.532 | 1.00 | 68.56 | C |
| ATOM | 176 | NE | ARG | A | 57 | 1.266 | 91.272 | 0.179 | 1.00 | 68.20 | N |
| ATOM | 177 | CZ | ARG | A | 57 | 0.777 | 90.086 | −0.208 | 1.00 | 68.80 | C |
| ATOM | 178 | NH1 | ARG | A | 57 | −0.551 | 89.870 | −0.291 | 1.00 | 69.12 | N |
| ATOM | 179 | NH2 | ARG | A | 57 | 1.616 | 89.106 | −0.517 | 1.00 | 69.04 | N |
| ATOM | 180 | C | ARG | A | 57 | −0.259 | 95.448 | 3.422 | 1.00 | 70.41 | C |
| ATOM | 181 | O | ARG | A | 57 | −1.430 | 95.146 | 3.171 | 1.00 | 70.64 | O |
| ATOM | 182 | N | VAL | A | 58 | 0.218 | 96.691 | 3.345 | 1.00 | 72.30 | N |
| ATOM | 183 | CA | VAL | A | 58 | −0.626 | 97.844 | 2.998 | 1.00 | 73.91 | C |
| ATOM | 184 | CB | VAL | A | 58 | 0.193 | 99.177 | 2.969 | 1.00 | 73.84 | C |
| ATOM | 185 | CG1 | VAL | A | 58 | −0.704 | 100.373 | 2.670 | 1.00 | 73.85 | C |
| ATOM | 186 | CG2 | VAL | A | 58 | 0.924 | 99.394 | 4.297 | 1.00 | 73.45 | C |
| ATOM | 187 | C | VAL | A | 58 | −1.348 | 97.602 | 1.666 | 1.00 | 74.98 | C |
| ATOM | 188 | O | VAL | A | 58 | −2.468 | 98.081 | 1.465 | 1.00 | 75.68 | O |
| ATOM | 189 | N | SER | A | 59 | −0.710 | 96.822 | 0.788 | 1.00 | 75.93 | N |
| ATOM | 190 | CA | SER | A | 59 | −1.268 | 96.456 | −0.521 | 1.00 | 76.51 | C |
| ATOM | 191 | CB | SER | A | 59 | −0.255 | 95.617 | −1.320 | 1.00 | 76.86 | C |
| ATOM | 192 | OG | SER | A | 59 | 1.103 | 96.061 | −1.049 | 1.00 | 78.49 | O |
| ATOM | 193 | C | SER | A | 59 | −2.617 | 95.721 | −0.460 | 1.00 | 76.40 | C |
| ATOM | 194 | O | SER | A | 59 | −3.382 | 95.775 | −1.422 | 1.00 | 76.81 | O |
| ATOM | 195 | N | ASP | A | 60 | −2.902 | 95.026 | 0.645 | 1.00 | 75.89 | N |
| ATOM | 196 | CA | ASP | A | 60 | −4.174 | 94.299 | 0.790 | 1.00 | 75.35 | C |
| ATOM | 197 | CB | ASP | A | 60 | −4.230 | 93.077 | −0.148 | 1.00 | 75.67 | C |
| ATOM | 198 | CG | ASP | A | 60 | −3.124 | 92.064 | 0.126 | 1.00 | 76.95 | C |
| ATOM | 199 | OD1 | ASP | A | 60 | −2.835 | 91.788 | 1.307 | 1.00 | 78.19 | O |
| ATOM | 200 | OD2 | ASP | A | 60 | −2.488 | 91.483 | −0.788 | 1.00 | 77.92 | O |
| ATOM | 201 | C | ASP | A | 60 | −4.543 | 93.872 | 2.217 | 1.00 | 74.33 | C |
| ATOM | 202 | O | ASP | A | 60 | −5.339 | 92.947 | 2.398 | 1.00 | 74.34 | O |
| ATOM | 203 | N | ASN | A | 61 | −3.965 | 94.541 | 3.215 | 1.00 | 72.99 | N |
| ATOM | 204 | CA | ASN | A | 61 | −4.194 | 94.219 | 4.633 | 1.00 | 71.45 | C |
| ATOM | 205 | CB | ASN | A | 61 | −5.599 | 94.651 | 5.074 | 1.00 | 72.10 | C |
| ATOM | 206 | CG | ASN | A | 61 | −5.790 | 96.158 | 5.026 | 1.00 | 73.42 | C |
| ATOM | 207 | OD1 | ASN | A | 61 | −5.333 | 96.885 | 5.926 | 1.00 | 74.58 | O |
| ATOM | 208 | ND2 | ASN | A | 61 | −6.471 | 96.636 | 3.975 | 1.00 | 74.28 | N |
| ATOM | 209 | C | ASN | A | 61 | −3.928 | 92.759 | 5.035 | 1.00 | 69.65 | C |
| ATOM | 210 | O | ASN | A | 61 | −4.535 | 92.242 | 5.975 | 1.00 | 69.76 | O |
| ATOM | 211 | N | LEU | A | 62 | −3.020 | 92.098 | 4.323 | 1.00 | 67.18 | N |
| ATOM | 212 | CA | LEU | A | 62 | −2.623 | 90.738 | 4.680 | 1.00 | 64.33 | C |
| ATOM | 213 | CB | LEU | A | 62 | −1.901 | 90.057 | 3.518 | 1.00 | 64.74 | C |
| ATOM | 214 | CG | LEU | A | 62 | −1.291 | 88.685 | 3.821 | 1.00 | 65.22 | C |
| ATOM | 215 | CD1 | LEU | A | 62 | −2.383 | 87.635 | 4.009 | 1.00 | 65.88 | C |
| ATOM | 216 | CD2 | LEU | A | 62 | −0.325 | 88.264 | 2.725 | 1.00 | 65.33 | C |
| ATOM | 217 | C | LEU | A | 62 | −1.698 | 90.766 | 5.883 | 1.00 | 61.89 | C |
| ATOM | 218 | O | LEU | A | 62 | −0.682 | 91.453 | 5.863 | 1.00 | 61.51 | O |
| ATOM | 219 | N | PRO | A | 63 | −2.044 | 90.010 | 6.920 | 1.00 | 59.57 | N |
| ATOM | 220 | CA | PRO | A | 63 | −1.164 | 89.840 | 8.083 | 1.00 | 57.75 | C |
| ATOM | 221 | CB | PRO | A | 63 | −1.963 | 88.888 | 8.983 | 1.00 | 57.73 | C |
| ATOM | 222 | CG | PRO | A | 63 | −3.376 | 89.106 | 8.573 | 1.00 | 58.58 | C |
| ATOM | 223 | CD | PRO | A | 63 | −3.303 | 89.261 | 7.080 | 1.00 | 59.38 | C |
| ATOM | 224 | C | PRO | A | 63 | 0.180 | 89.221 | 7.694 | 1.00 | 55.60 | C |
| ATOM | 225 | O | PRO | A | 63 | 0.211 | 88.163 | 7.075 | 1.00 | 55.56 | O |
| ATOM | 226 | N | VAL | A | 64 | 1.274 | 89.902 | 8.025 | 1.00 | 53.21 | N |
| ATOM | 227 | CA | VAL | A | 64 | 2.609 | 89.375 | 7.773 | 1.00 | 50.67 | C |
| ATOM | 228 | CB | VAL | A | 64 | 3.306 | 90.097 | 6.590 | 1.00 | 50.93 | C |
| ATOM | 229 | CG1 | VAL | A | 64 | 2.441 | 90.040 | 5.326 | 1.00 | 50.56 | C |
| ATOM | 230 | CG2 | VAL | A | 64 | 3.641 | 91.537 | 6.943 | 1.00 | 49.88 | C |
| ATOM | 231 | C | VAL | A | 64 | 3.492 | 89.445 | 9.025 | 1.00 | 49.15 | C |
| ATOM | 232 | O | VAL | A | 64 | 3.175 | 90.150 | 9.981 | 1.00 | 49.44 | O |
| ATOM | 233 | N | ALA | A | 65 | 4.587 | 88.692 | 9.015 | 1.00 | 46.68 | N |
| ATOM | 234 | CA | ALA | A | 65 | 5.604 | 88.774 | 10.046 | 1.00 | 44.84 | C |
| ATOM | 235 | CB | ALA | A | 65 | 5.881 | 87.384 | 10.654 | 1.00 | 45.04 | C |
| ATOM | 236 | C | ALA | A | 65 | 6.834 | 89.315 | 9.356 | 1.00 | 43.92 | C |
| ATOM | 237 | O | ALA | A | 65 | 7.123 | 88.912 | 8.218 | 1.00 | 43.40 | O |
| ATOM | 238 | N | ILE | A | 66 | 7.547 | 90.233 | 10.012 | 1.00 | 42.88 | N |
| ATOM | 239 | CA | ILE | A | 66 | 8.716 | 90.883 | 9.405 | 1.00 | 42.53 | C |
| ATOM | 240 | CB | ILE | A | 66 | 8.494 | 92.410 | 9.252 | 1.00 | 43.51 | C |
| ATOM | 241 | CG1 | ILE | A | 66 | 7.260 | 92.679 | 8.383 | 1.00 | 44.11 | C |
| ATOM | 242 | CD1 | ILE | A | 66 | 6.685 | 94.112 | 8.501 | 1.00 | 47.03 | C |
| ATOM | 243 | CG2 | ILE | A | 66 | 9.704 | 93.067 | 8.636 | 1.00 | 42.39 | C |
| ATOM | 244 | C | ILE | A | 66 | 9.958 | 90.572 | 10.214 | 1.00 | 42.61 | C |
| ATOM | 245 | O | ILE | A | 66 | 10.119 | 91.057 | 11.342 | 1.00 | 41.89 | O |
| ATOM | 246 | N | LYS | A | 67 | 10.820 | 89.731 | 9.641 | 1.00 | 41.21 | N |
| ATOM | 247 | CA | LYS | A | 67 | 11.971 | 89.193 | 10.353 | 1.00 | 41.66 | C |
| ATOM | 248 | CB | LYS | A | 67 | 12.052 | 87.664 | 10.164 | 1.00 | 41.05 | C |
| ATOM | 249 | CG | LYS | A | 67 | 13.288 | 87.013 | 10.761 | 1.00 | 42.61 | C |
| ATOM | 250 | CD | LYS | A | 67 | 13.165 | 85.495 | 10.666 | 1.00 | 44.83 | C |
| ATOM | 251 | CE | LYS | A | 67 | 14.213 | 84.780 | 11.488 | 1.00 | 46.29 | C |
| ATOM | 252 | NZ | LYS | A | 67 | 14.165 | 83.309 | 11.228 | 1.00 | 46.83 | N |
| ATOM | 253 | C | LYS | A | 67 | 13.243 | 89.833 | 9.867 | 1.00 | 41.57 | C |
| ATOM | 254 | O | LYS | A | 67 | 13.548 | 89.773 | 8.671 | 1.00 | 40.97 | O |
| ATOM | 255 | N | HIS | A | 68 | 13.988 | 90.415 | 10.807 | 1.00 | 41.97 | N |
| ATOM | 256 | CA | HTS | A | 68 | 15.254 | 91.087 | 10.553 | 1.00 | 43.17 | C |
| ATOM | 257 | CB | HIS | A | 68 | 15.343 | 92.419 | 11.318 | 1.00 | 42.46 | C |
| ATOM | 258 | CG | HIS | A | 68 | 14.352 | 93.440 | 10.858 | 1.00 | 40.77 | C |
| ATOM | 259 | ND1 | HIS | A | 68 | 13.018 | 93.384 | 11.203 | 1.00 | 43.58 | N |
| ATOM | 260 | CE1 | HIS | A | 68 | 12.376 | 94.393 | 10.640 | 1.00 | 41.67 | C |
| ATOM | 261 | NE2 | HIS | A | 68 | 13.247 | 95.100 | 9.942 | 1.00 | 41.02 | N |
| ATOM | 262 | CD2 | HIS | A | 68 | 14.489 | 94.522 | 10.062 | 1.00 | 37.93 | C |
| ATOM | 263 | C | HIS | A | 68 | 16.408 | 90.217 | 10.960 | 1.00 | 45.01 | C |
| ATOM | 264 | O | HIS | A | 68 | 16.466 | 89.744 | 12.089 | 1.00 | 44.97 | O |
| ATOM | 265 | N | VAL | A | 69 | 17.340 | 90.027 | 10.030 | 1.00 | 46.61 | N |
| ATOM | 266 | CA | VAL | A | 69 | 18.516 | 89.225 | 10.272 | 1.00 | 49.40 | C |
| ATOM | 267 | CB | VAL | A | 69 | 18.538 | 87.969 | 9.359 | 1.00 | 49.48 | C |
| ATOM | 268 | CG1 | VAL | A | 69 | 19.738 | 87.093 | 9.675 | 1.00 | 50.89 | C |
| ATOM | 269 | CG2 | VAL | A | 69 | 17.266 | 87.146 | 9.529 | 1.00 | 49.70 | C |
| ATOM | 270 | C | VAL | A | 69 | 19.746 | 90.103 | 10.038 | 1.00 | 51.36 | C |
| ATOM | 271 | O | VAL | A | 69 | 19.879 | 90.721 | 8.983 | 1.00 | 50.89 | O |
| ATOM | 272 | N | GLU | A | 70 | 20.634 | 90.162 | 11.026 | 1.00 | 53.97 | N |
| ATOM | 273 | CA | GLU | A | 70 | 21.870 | 90.924 | 10.896 | 1.00 | 57.27 | C |
| ATOM | 274 | CB | GLU | A | 70 | 22.480 | 91.219 | 12.272 | 1.00 | 57.98 | C |
| ATOM | 275 | CG | GLU | A | 70 | 21.674 | 92.205 | 13.105 | 1.00 | 61.81 | C |
| ATOM | 276 | CD | GLU | A | 70 | 22.524 | 93.240 | 13.839 | 1.00 | 66.09 | C |
| ATOM | 277 | OE1 | GLU | A | 70 | 21.982 | 93.928 | 14.744 | 1.00 | 67.00 | O |
| ATOM | 278 | OE2 | GLU | A | 70 | 23.729 | 93.377 | 13.518 | 1.00 | 68.05 | O |
| ATOM | 279 | C | GLU | A | 70 | 22.861 | 90.148 | 10.057 | 1.00 | 58.15 | C |
| ATOM | 280 | O | GLU | A | 70 | 23.115 | 88.977 | 10.332 | 1.00 | 57.86 | O |
| ATOM | 281 | N | LYS | A | 71 | 23.420 | 90.807 | 9.041 | 1.00 | 60.40 | N |
| ATOM | 282 | CA | LYS | A | 71 | 24.433 | 90.193 | 8.174 | 1.00 | 62.67 | C |
| ATOM | 283 | CB | LYS | A | 71 | 24.982 | 91.207 | 7.166 | 1.00 | 62.59 | C |
| ATOM | 284 | CG | LYS | A | 71 | 23.999 | 91.544 | 6.056 | 1.00 | 63.22 | C |
| ATOM | 285 | CD | LYS | A | 71 | 24.634 | 92.387 | 4.973 | 1.00 | 64.60 | C |
| ATOM | 286 | CE | LYS | A | 71 | 23.644 | 92.635 | 3.848 | 1.00 | 65.13 | C |
| ATOM | 287 | NZ | LYS | A | 71 | 24.159 | 93.586 | 2.831 | 1.00 | 66.01 | N |
| ATOM | 288 | C | LYS | A | 71 | 25.567 | 89.589 | 8.987 | 1.00 | 64.37 | C |
| ATOM | 289 | O | LYS | A | 71 | 25.990 | 88.462 | 8.737 | 1.00 | 64.24 | O |
| ATOM | 290 | N | ASP | A | 72 | 26.029 | 90.336 | 9.986 | 1.00 | 67.27 | N |
| ATOM | 291 | CA | ASP | A | 72 | 27.153 | 89.928 | 10.820 | 1.00 | 70.03 | C |
| ATOM | 292 | CB | ASP | A | 72 | 27.483 | 91.037 | 11.814 | 1.00 | 70.83 | C |
| ATOM | 293 | CG | ASP | A | 72 | 28.294 | 92.162 | 11.177 | 1.00 | 73.07 | C |
| ATOM | 294 | OD1 | ASP | A | 72 | 27.828 | 92.764 | 10.174 | 1.00 | 75.44 | O |
| ATOM | 295 | OD2 | ASP | A | 72 | 29.412 | 92.511 | 11.611 | 1.00 | 74.89 | O |
| ATOM | 296 | C | ASP | A | 72 | 26.923 | 88.614 | 11.551 | 1.00 | 71.40 | C |
| ATOM | 297 | O | ASP | A | 72 | 27.875 | 87.895 | 11.851 | 1.00 | 71.59 | O |
| ATOM | 298 | N | ARG | A | 73 | 25.658 | 88.287 | 11.805 | 1.00 | 73.23 | N |
| ATOM | 299 | CA | ARG | A | 73 | 25.304 | 87.068 | 12.540 | 1.00 | 74.86 | C |
| ATOM | 300 | CB | ARG | A | 73 | 24.241 | 87.380 | 13.602 | 1.00 | 75.53 | C |
| ATOM | 301 | CG | ARG | A | 73 | 24.741 | 88.293 | 14.718 | 1.00 | 79.03 | C |
| ATOM | 302 | CD | ARG | A | 73 | 23.959 | 88.151 | 16.043 | 1.00 | 84.58 | C |
| ATOM | 303 | NE | ARG | A | 73 | 23.692 | 86.752 | 16.394 | 1.00 | 88.34 | N |
| ATOM | 304 | CZ | ARG | A | 73 | 24.598 | 85.901 | 16.878 | 1.00 | 89.85 | C |
| ATOM | 305 | NH1 | ARG | A | 73 | 25.857 | 86.293 | 17.083 | 1.00 | 90.48 | N |
| ATOM | 306 | NH2 | ARG | A | 73 | 24.239 | 84.650 | 17.161 | 1.00 | 90.61 | N |
| ATOM | 307 | C | ARG | A | 73 | 24.839 | 85.929 | 11.630 | 1.00 | 75.02 | C |
| ATOM | 308 | O | ARG | A | 73 | 24.067 | 85.067 | 12.054 | 1.00 | 75.13 | O |
| ATOM | 309 | N | ILE | A | 74 | 25.321 | 85.926 | 10.386 | 1.00 | 75.26 | N |
| ATOM | 310 | CA | ILE | A | 74 | 24.969 | 84.885 | 9.420 | 1.00 | 75.36 | C |
| ATOM | 311 | CB | ILE | A | 74 | 24.359 | 85.503 | 8.127 | 1.00 | 75.17 | C |
| ATOM | 312 | CG1 | ILE | A | 74 | 23.188 | 86.425 | 8.465 | 1.00 | 74.84 | C |
| ATOM | 313 | CD1 | ILE | A | 74 | 22.660 | 87.204 | 7.280 | 1.00 | 75.37 | C |
| ATOM | 314 | CG2 | ILE | A | 74 | 23.893 | 84.408 | 7.166 | 1.00 | 74.86 | C |
| ATOM | 315 | C | ILE | A | 74 | 26.189 | 84.022 | 9.088 | 1.00 | 75.92 | C |
| ATOM | 316 | O | ILE | A | 74 | 27.201 | 84.519 | 8.578 | 1.00 | 76.08 | O |
| ATOM | 317 | N | SER | A | 75 | 26.090 | 82.730 | 9.386 | 1.00 | 76.26 | N |
| ATOM | 318 | CA | SER | A | 75 | 27.155 | 81.784 | 9.072 | 1.00 | 76.63 | C |
| ATOM | 319 | CB | SER | A | 75 | 27.184 | 80.641 | 10.094 | 1.00 | 77.05 | C |
| ATOM | 320 | OG | SER | A | 75 | 26.007 | 79.839 | 10.009 | 1.00 | 78.24 | O |
| ATOM | 321 | C | SER | A | 75 | 26.990 | 81.226 | 7.660 | 1.00 | 76.35 | C |
| ATOM | 322 | O | SER | A | 75 | 27.918 | 81.285 | 6.855 | 1.00 | 76.40 | O |
| ATOM | 323 | N | ASP | A | 76 | 25.798 | 80.703 | 7.372 | 1.00 | 75.95 | N |
| ATOM | 324 | CA | ASP | A | 76 | 25.512 | 80.025 | 6.109 | 1.00 | 75.64 | C |
| ATOM | 325 | CB | ASP | A | 76 | 24.528 | 78.875 | 6.332 | 1.00 | 76.36 | C |
| ATOM | 326 | CG | ASP | A | 76 | 25.112 | 77.756 | 7.157 | 1.00 | 78.38 | C |
| ATOM | 327 | OD1 | ASP | A | 76 | 25.828 | 76.906 | 6.579 | 1.00 | 80.43 | O |
| ATOM | 328 | OD2 | ASP | A | 76 | 24.900 | 77.642 | 8.391 | 1.00 | 81.66 | O |
| ATOM | 329 | C | ASP | A | 76 | 24.948 | 80.952 | 5.043 | 1.00 | 74.58 | C |
| ATOM | 330 | O | ASP | A | 76 | 23.946 | 81.635 | 5.262 | 1.00 | 74.37 | O |
| ATOM | 331 | N | TRP | A | 77 | 25.592 | 80.945 | 3.879 | 1.00 | 73.73 | N |
| ATOM | 332 | CA | TRP | A | 77 | 25.148 | 81.728 | 2.730 | 1.00 | 72.88 | C |
| ATOM | 333 | CB | TRP | A | 77 | 26.159 | 82.826 | 2.398 | 1.00 | 72.08 | C |
| ATOM | 334 | CG | TRP | A | 77 | 26.345 | 83.854 | 3.455 | 1.00 | 68.72 | C |
| ATOM | 335 | CD1 | TRP | A | 77 | 27.105 | 83.748 | 4.582 | 1.00 | 67.14 | C |
| ATOM | 336 | NE1 | TRP | A | 77 | 27.038 | 84.911 | 5.313 | 1.00 | 66.79 | N |
| ATOM | 337 | CE2 | TRP | A | 77 | 26.228 | 85.800 | 4.657 | 1.00 | 66.47 | C |
| ATOM | 338 | CD2 | TRP | A | 77 | 25.776 | 85.163 | 3.478 | 1.00 | 66.40 | C |
| ATOM | 339 | CE3 | TRP | A | 77 | 24.926 | 85.870 | 2.620 | 1.00 | 65.70 | C |
| ATOM | 340 | CZ3 | TRP | A | 77 | 24.557 | 87.169 | 2.958 | 1.00 | 65.66 | C |
| ATOM | 341 | CH2 | TRP | A | 77 | 25.023 | 87.770 | 4.139 | 1.00 | 65.79 | C |
| ATOM | 342 | CZ2 | TRP | A | 77 | 25.858 | 87.104 | 5.000 | 1.00 | 65.98 | C |
| ATOM | 343 | C | TRP | A | 77 | 25.020 | 80.808 | 1.529 | 1.00 | 73.58 | C |
| ATOM | 344 | O | TRP | A | 77 | 25.727 | 79.802 | 1.434 | 1.00 | 73.41 | O |
| ATOM | 345 | N | GLY | A | 78 | 24.127 | 81.159 | 0.610 | 1.00 | 74.27 | N |
| ATOM | 346 | CA | GLY | A | 78 | 23.959 | 80.407 | −0.623 | 1.00 | 75.77 | C |
| ATOM | 347 | C | GLY | A | 78 | 23.491 | 81.313 | −1.740 | 1.00 | 77.06 | C |
| ATOM | 348 | O | GLY | A | 78 | 23.426 | 82.534 | −1.567 | 1.00 | 77.12 | O |
| ATOM | 349 | N | GLU | A | 79 | 23.157 | 80.725 | −2.887 | 1.00 | 78.52 | N |
| ATOM | 350 | CA | GLU | A | 79 | 22.685 | 81.517 | −4.022 | 1.00 | 80.32 | C |
| ATOM | 351 | CB | GLU | A | 79 | 23.710 | 81.532 | −5.170 | 1.00 | 80.86 | C |
| ATOM | 352 | CG | GLU | A | 79 | 24.083 | 80.152 | −5.723 | 1.00 | 83.43 | C |
| ATOM | 353 | CD | GLU | A | 79 | 24.713 | 80.234 | −7.108 | 1.00 | 85.86 | C |
| ATOM | 354 | OE1 | GLU | A | 79 | 25.813 | 80.822 | −7.235 | 1.00 | 86.43 | O |
| ATOM | 355 | OE2 | GLU | A | 79 | 24.107 | 79.708 | −8.071 | 1.00 | 86.75 | O |
| ATOM | 356 | C | GLU | A | 79 | 21.311 | 81.091 | −4.518 | 1.00 | 80.80 | C |
| ATOM | 357 | O | GLU | A | 79 | 20.948 | 79.917 | −4.453 | 1.00 | 80.46 | O |
| ATOM | 358 | N | LEU | A | 80 | 20.558 | 82.069 | −5.012 | 1.00 | 81.81 | N |
| ATOM | 359 | CA | LEU | A | 80 | 19.233 | 81.837 | −5.582 | 1.00 | 82.81 | C |
| ATOM | 360 | CB | LEU | A | 80 | 18.441 | 83.152 | −5.596 | 1.00 | 82.78 | C |
| ATOM | 361 | CG | LEU | A | 80 | 18.289 | 83.870 | −4.254 | 1.00 | 83.30 | C |
| ATOM | 362 | CD1 | LEU | A | 80 | 17.545 | 85.189 | −4.432 | 1.00 | 83.40 | C |
| ATOM | 363 | CD2 | LEU | A | 80 | 17.588 | 82.965 | −3.238 | 1.00 | 83.57 | C |
| ATOM | 364 | C | LEU | A | 80 | 19.343 | 81.256 | −6.998 | 1.00 | 83.29 | C |
| ATOM | 365 | O | LEU | A | 80 | 20.440 | 81.256 | −7.570 | 1.00 | 83.54 | O |
| ATOM | 366 | N | PRO | A | 81 | 18.235 | 80.753 | −7.567 | 1.00 | 83.78 | N |
| ATOM | 367 | CA | PRO | A | 81 | 18.221 | 80.340 | −8.986 | 1.00 | 83.97 | C |
| ATOM | 368 | CB | PRO | A | 81 | 16.758 | 79.937 | −9.218 | 1.00 | 83.94 | C |
| ATOM | 369 | CG | PRO | A | 81 | 16.267 | 79.535 | −7.871 | 1.00 | 84.00 | C |
| ATOM | 370 | CD | PRO | A | 81 | 16.927 | 80.513 | −6.923 | 1.00 | 83.86 | C |
| ATOM | 371 | C | PRO | A | 81 | 18.623 | 81.488 | −9.926 | 1.00 | 84.09 | C |
| ATOM | 372 | O | PRO | A | 81 | 18.910 | 81.267 | −11.102 | 1.00 | 84.07 | O |
| ATOM | 373 | N | ASN | A | 82 | 18.644 | 82.700 | −9.376 | 1.00 | 84.20 | N |
| ATOM | 374 | CA | ASN | A | 82 | 19.070 | 83.916 | −10.064 | 1.00 | 83.96 | C |
| ATOM | 375 | CB | ASN | A | 82 | 18.276 | 85.106 | −9.492 | 1.00 | 84.25 | C |
| ATOM | 376 | CG | ASN | A | 82 | 18.738 | 86.449 | −10.026 | 1.00 | 85.14 | C |
| ATOM | 377 | OD1 | ASN | A | 82 | 18.869 | 86.643 | −11.241 | 1.00 | 85.89 | O |
| ATOM | 378 | ND2 | ASN | A | 82 | 18.979 | 87.393 | −9.115 | 1.00 | 84.89 | N |
| ATOM | 379 | C | ASN | A | 82 | 20.586 | 84.137 | −9.935 | 1.00 | 83.40 | C |
| ATOM | 380 | O | ASN | A | 82 | 21.190 | 84.889 | −10.709 | 1.00 | 83.24 | O |
| ATOM | 381 | N | GLY | A | 83 | 21.191 | 83.461 | −8.958 | 1.00 | 82.90 | N |
| ATOM | 382 | CA | GLY | A | 83 | 22.597 | 83.634 | −8.626 | 1.00 | 82.10 | C |
| ATOM | 383 | C | GLY | A | 83 | 22.845 | 84.924 | −7.863 | 1.00 | 81.49 | C |
| ATOM | 384 | O | GLY | A | 83 | 23.382 | 85.883 | −8.430 | 1.00 | 81.74 | O |
| ATOM | 385 | N | THR | A | 84 | 22.437 | 84.944 | −6.590 | 1.00 | 80.61 | N |
| ATOM | 386 | CA | THR | A | 84 | 22.609 | 86.097 | −5.687 | 1.00 | 79.41 | C |
| ATOM | 387 | CB | THR | A | 84 | 21.290 | 86.893 | −5.543 | 1.00 | 79.60 | C |
| ATOM | 388 | OG1 | THR | A | 84 | 20.718 | 87.127 | −6.836 | 1.00 | 80.05 | O |
| ATOM | 389 | CG2 | THR | A | 84 | 21.561 | 88.322 | −5.007 | 1.00 | 79.91 | C |
| ATOM | 390 | C | THR | A | 84 | 23.081 | 85.643 | −4.302 | 1.00 | 78.07 | C |
| ATOM | 391 | O | THR | A | 84 | 22.728 | 84.557 | −3.841 | 1.00 | 78.47 | O |
| ATOM | 392 | N | ARG | A | 85 | 23.866 | 86.489 | −3.643 | 1.00 | 75.99 | N |
| ATOM | 393 | CA | ARG | A | 85 | 24.443 | 86.177 | −2.338 | 1.00 | 73.77 | C |
| ATOM | 394 | CB | ARG | A | 85 | 25.768 | 86.943 | −2.184 | 1.00 | 74.21 | C |
| ATOM | 395 | CG | ARG | A | 85 | 26.453 | 86.829 | −0.833 | 1.00 | 75.15 | C |
| ATOM | 396 | CD | ARG | A | 85 | 27.404 | 85.638 | −0.725 | 1.00 | 75.91 | C |
| ATOM | 397 | NE | ARG | A | 85 | 28.213 | 85.731 | 0.487 | 1.00 | 76.30 | N |
| ATOM | 398 | CZ | ARG | A | 85 | 28.957 | 84.739 | 0.972 | 1.00 | 77.02 | C |
| ATOM | 399 | NH1 | ARG | A | 85 | 29.005 | 83.560 | 0.352 | 1.00 | 76.24 | N |
| ATOM | 400 | NH2 | ARG | A | 85 | 29.655 | 84.929 | 2.086 | 1.00 | 77.19 | N |
| ATOM | 401 | C | ARG | A | 85 | 23.457 | 86.502 | −1.199 | 1.00 | 71.72 | C |
| ATOM | 402 | O | ARG | A | 85 | 23.415 | 87.632 | −0.696 | 1.00 | 71.91 | O |
| ATOM | 403 | N | VAL | A | 86 | 22.653 | 85.514 | −0.809 | 1.00 | 68.61 | N |
| ATOM | 404 | CA | VAL | A | 86 | 21.660 | 85.693 | 0.262 | 1.00 | 65.46 | C |
| ATOM | 405 | CB | VAL | A | 86 | 20.191 | 85.642 | −0.265 | 1.00 | 65.32 | C |
| ATOM | 406 | CG1 | VAL | A | 86 | 19.977 | 86.633 | −1.394 | 1.00 | 64.79 | C |
| ATOM | 407 | CG2 | VAL | A | 86 | 19.822 | 84.250 | −0.709 | 1.00 | 65.00 | C |
| ATOM | 408 | C | VAL | A | 86 | 21.866 | 84.656 | 1.372 | 1.00 | 63.06 | C |
| ATOM | 409 | O | VAL | A | 86 | 22.543 | 83.649 | 1.144 | 1.00 | 63.20 | O |
| ATOM | 410 | N | PRO | A | 87 | 21.301 | 84.887 | 2.563 | 1.00 | 60.50 | N |
| ATOM | 411 | CA | PRO | A | 87 | 21.399 | 83.907 | 3.649 | 1.00 | 58.23 | C |
| ATOM | 412 | CB | PRO | A | 87 | 20.570 | 84.544 | 4.774 | 1.00 | 58.22 | C |
| ATOM | 413 | CG | PRO | A | 87 | 20.590 | 85.999 | 4.478 | 1.00 | 59.47 | C |
| ATOM | 414 | CD | PRO | A | 87 | 20.535 | 86.082 | 2.986 | 1.00 | 60.10 | C |
| ATOM | 415 | C | PRO | A | 87 | 20.797 | 82.564 | 3.237 | 1.00 | 56.09 | C |
| ATOM | 416 | O | PRO | A | 87 | 19.802 | 82.524 | 2.513 | 1.00 | 55.24 | O |
| ATOM | 417 | N | MET | A | 88 | 21.416 | 81.479 | 3.681 | 1.00 | 54.23 | N |
| ATOM | 418 | CA | MET | A | 88 | 20.866 | 80.142 | 3.458 | 1.00 | 53.19 | C |
| ATOM | 419 | CB | MET | A | 88 | 21.638 | 79.111 | 4.295 | 1.00 | 54.18 | C |
| ATOM | 420 | CG | MET | A | 88 | 21.273 | 77.645 | 4.025 | 1.00 | 57.50 | C |
| ATOM | 421 | SD | MET | A | 88 | 21.341 | 77.213 | 2.247 | 1.00 | 65.32 | S |
| ATOM | 422 | CE | MET | A | 88 | 23.113 | 77.148 | 2.002 | 1.00 | 62.88 | C |
| ATOM | 423 | C | MET | A | 88 | 19.363 | 80.103 | 3.775 | 1.00 | 50.99 | C |
| ATOM | 424 | O | MET | A | 88 | 18.565 | 79.594 | 2.979 | 1.00 | 49.59 | O |
| ATOM | 425 | N | GLU | A | 89 | 18.982 | 80.688 | 4.918 | 1.00 | 48.97 | N |
| ATOM | 426 | CA | GLU | A | 89 | 17.575 | 80.754 | 5.317 | 1.00 | 46.86 | C |
| ATOM | 427 | CB | GLU | A | 89 | 17.392 | 81.686 | 6.522 | 1.00 | 45.85 | C |
| ATOM | 428 | CG | GLU | A | 89 | 15.944 | 81.803 | 6.991 | 1.00 | 45.51 | C |
| ATOM | 429 | CD | GLU | A | 89 | 15.803 | 82.541 | 8.303 | 1.00 | 44.03 | C |
| ATOM | 430 | OE1 | GLU | A | 89 | 16.819 | 83.008 | 8.856 | 1.00 | 47.34 | O |
| ATOM | 431 | OE2 | GLU | A | 89 | 14.671 | 82.638 | 8.790 | 1.00 | 44.02 | O |
| ATOM | 432 | C | GLU | A | 89 | 16.653 | 81.168 | 4.171 | 1.00 | 46.50 | C |
| ATOM | 433 | O | GLU | A | 89 | 15.612 | 80.548 | 3.962 | 1.00 | 46.41 | O |
| ATOM | 434 | N | VAL | A | 90 | 17.031 | 82.215 | 3.429 | 1.00 | 46.05 | N |
| ATOM | 435 | CA | VAL | A | 90 | 16.243 | 82.669 | 2.275 | 1.00 | 45.86 | C |
| ATOM | 436 | CB | VAL | A | 90 | 16.759 | 84.018 | 1.725 | 1.00 | 46.25 | C |
| ATOM | 437 | CG1 | VAL | A | 90 | 15.966 | 84.431 | 0.491 | 1.00 | 45.50 | C |
| ATOM | 438 | CG2 | VAL | A | 90 | 16.663 | 85.102 | 2.800 | 1.00 | 46.57 | C |
| ATOM | 439 | C | VAL | A | 90 | 16.234 | 81.639 | 1.137 | 1.00 | 45.66 | C |
| ATOM | 440 | O | VAL | A | 90 | 15.210 | 81.399 | 0.525 | 1.00 | 45.75 | O |
| ATOM | 441 | N | VAL | A | 91 | 17.389 | 81.053 | 0.851 | 1.00 | 45.99 | N |
| ATOM | 442 | CA | VAL | A | 91 | 17.490 | 80.034 | −0.197 | 1.00 | 46.17 | C |
| ATOM | 443 | CB | VAL | A | 91 | 18.913 | 79.465 | −0.279 | 1.00 | 46.54 | C |
| ATOM | 444 | CG1 | VAL | A | 91 | 18.975 | 78.292 | −1.284 | 1.00 | 47.68 | C |
| ATOM | 445 | CG2 | VAL | A | 91 | 19.892 | 80.556 | −0.674 | 1.00 | 48.34 | C |
| ATOM | 446 | C | VAL | A | 91 | 16.496 | 78.909 | 0.094 | 1.00 | 44.94 | C |
| ATOM | 447 | O | VAL | A | 91 | 15.631 | 78.603 | −0.729 | 1.00 | 45.36 | O |
| ATOM | 448 | N | LEU | A | 92 | 16.591 | 78.352 | 1.302 | 1.00 | 43.94 | N |
| ATOM | 449 | CA | LEU | A | 92 | 15.704 | 77.260 | 1.749 | 1.00 | 42.33 | C |
| ATOM | 450 | CB | LEU | A | 92 | 16.106 | 76.772 | 3.137 | 1.00 | 40.99 | C |
| ATOM | 451 | CG | LEU | A | 92 | 17.577 | 76.417 | 3.316 | 1.00 | 40.75 | C |
| ATOM | 452 | CD1 | LEU | A | 92 | 17.798 | 75.867 | 4.711 | 1.00 | 37.63 | C |
| ATOM | 453 | CD2 | LEU | A | 92 | 18.061 | 75.410 | 2.247 | 1.00 | 40.38 | C |
| ATOM | 454 | C | LEU | A | 92 | 14.245 | 77.641 | 1.742 | 1.00 | 42.09 | C |
| ATOM | 455 | O | LEU | A | 92 | 13.401 | 76.888 | 1.243 | 1.00 | 41.96 | O |
| ATOM | 456 | N | LEU | A | 93 | 13.936 | 78.812 | 2.289 | 1.00 | 42.17 | N |
| ATOM | 457 | CA | LEU | A | 93 | 12.556 | 79.280 | 2.328 | 1.00 | 43.42 | C |
| ATOM | 458 | CB | LEU | A | 93 | 12.461 | 80.632 | 3.051 | 1.00 | 42.52 | C |
| ATOM | 459 | CG | LEU | A | 93 | 12.416 | 80.645 | 4.589 | 1.00 | 42.30 | C |
| ATOM | 460 | CD1 | LEU | A | 93 | 12.576 | 82.074 | 5.095 | 1.00 | 39.64 | C |
| ATOM | 461 | CD2 | LEU | A | 93 | 11.117 | 80.069 | 5.107 | 1.00 | 39.20 | C |
| ATOM | 462 | C | LEU | A | 93 | 11.947 | 79.382 | 0.921 | 1.00 | 44.51 | C |
| ATOM | 463 | O | LEU | A | 93 | 10.823 | 78.940 | 0.691 | 1.00 | 44.42 | O |
| ATOM | 464 | N | LYS | A | 94 | 12.690 | 79.981 | −0.012 | 1.00 | 46.08 | N |
| ATOM | 465 | CA | LYS | A | 94 | 12.222 | 80.098 | −1.391 | 1.00 | 47.69 | C |
| ATOM | 466 | CB | LYS | A | 94 | 13.266 | 80.807 | −2.254 | 1.00 | 48.80 | C |
| ATOM | 467 | CG | LYS | A | 94 | 13.146 | 82.329 | −2.195 | 1.00 | 52.89 | C |
| ATOM | 468 | CD | LYS | A | 94 | 14.133 | 83.009 | −3.126 | 1.00 | 57.20 | C |
| ATOM | 469 | CE | LYS | A | 94 | 13.761 | 82.810 | −4.598 | 1.00 | 58.87 | C |
| ATOM | 470 | NZ | LYS | A | 94 | 12.411 | 83.360 | −4.919 | 1.00 | 60.23 | N |
| ATOM | 471 | C | LYS | A | 94 | 11.903 | 78.730 | −1.981 | 1.00 | 47.52 | C |
| ATOM | 472 | O | LYS | A | 94 | 10.870 | 78.564 | −2.633 | 1.00 | 48.02 | O |
| ATOM | 473 | N | LYS | A | 95 | 12.792 | 77.766 | −1.733 | 1.00 | 47.55 | N |
| ATOM | 474 | CA | LYS | A | 95 | 12.615 | 76.380 | −2.185 | 1.00 | 48.35 | C |
| ATOM | 475 | CB | LYS | A | 95 | 13.836 | 75.536 | −1.829 | 1.00 | 48.23 | C |
| ATOM | 476 | CG | LYS | A | 95 | 15.023 | 75.801 | −2.747 | 1.00 | 48.74 | C |
| ATOM | 477 | CD | LYS | A | 95 | 16.293 | 75.188 | −2.212 | 1.00 | 50.92 | C |
| ATOM | 478 | CE | LYS | A | 95 | 16.392 | 73.705 | −2.529 | 1.00 | 53.76 | C |
| ATOM | 479 | NZ | LYS | A | 95 | 16.339 | 73.414 | −3.999 | 1.00 | 55.34 | N |
| ATOM | 480 | C | LYS | A | 95 | 11.351 | 75.706 | −1.659 | 1.00 | 48.52 | C |
| ATOM | 481 | O | LYS | A | 95 | 10.770 | 74.872 | −2.358 | 1.00 | 48.90 | O |
| ATOM | 482 | N | VAL | A | 96 | 10.921 | 76.056 | −0.444 | 1.00 | 48.20 | N |
| ATOM | 483 | CA | VAL | A | 96 | 9.759 | 75.395 | 0.149 | 1.00 | 48.68 | C |
| ATOM | 484 | CB | VAL | A | 96 | 10.001 | 74.989 | 1.620 | 1.00 | 48.64 | C |
| ATOM | 485 | CG1 | VAL | A | 96 | 11.105 | 73.977 | 1.718 | 1.00 | 45.61 | C |
| ATOM | 486 | CG2 | VAL | A | 96 | 10.301 | 76.238 | 2.498 | 1.00 | 47.01 | C |
| ATOM | 487 | C | VAL | A | 96 | 8.469 | 76.211 | 0.082 | 1.00 | 50.79 | C |
| ATOM | 488 | O | VAL | A | 96 | 7.412 | 75.751 | 0.544 | 1.00 | 50.14 | O |
| ATOM | 489 | N | SER | A | 97 | 8.547 | 77.419 | −0.476 | 1.00 | 52.75 | N |
| ATOM | 490 | CA | SER | A | 97 | 7.384 | 78.301 | −0.523 | 1.00 | 55.97 | C |
| ATOM | 491 | CB | SER | A | 97 | 7.820 | 79.751 | −0.306 | 1.00 | 56.04 | C |
| ATOM | 492 | OG | SER | A | 97 | 8.364 | 79.912 | 0.999 | 1.00 | 53.48 | O |
| ATOM | 493 | C | SER | A | 97 | 6.572 | 78.124 | −1.816 | 1.00 | 58.83 | C |
| ATOM | 494 | O | SER | A | 97 | 7.097 | 78.270 | −2.921 | 1.00 | 60.33 | O |
| ATOM | 495 | N | SER | A | 98 | 5.294 | 77.767 | −1.667 | 1.00 | 61.85 | N |
| ATOM | 496 | CA | SER | A | 98 | 4.397 | 77.478 | −2.805 | 1.00 | 63.61 | C |
| ATOM | 497 | CB | SER | A | 98 | 5.081 | 76.573 | −3.822 | 1.00 | 63.67 | C |
| ATOM | 498 | OG | SER | A | 98 | 5.317 | 75.300 | −3.246 | 1.00 | 63.53 | O |
| ATOM | 499 | C | SER | A | 98 | 3.120 | 76.797 | −2.304 | 1.00 | 65.08 | C |
| ATOM | 500 | O | SER | A | 98 | 2.764 | 76.902 | −1.125 | 1.00 | 65.04 | O |
| ATOM | 501 | N | GLY | A | 99 | 2.442 | 76.091 | −3.204 | 1.00 | 66.21 | N |
| ATOM | 502 | CA | GLY | A | 99 | 1.192 | 75.403 | −2.892 | 1.00 | 67.46 | C |
| ATOM | 503 | C | GLY | A | 99 | 0.948 | 74.924 | −1.464 | 1.00 | 67.88 | C |
| ATOM | 504 | O | GLY | A | 99 | −0.086 | 75.258 | −0.860 | 1.00 | 68.29 | O |
| ATOM | 505 | N | PHE | A | 100 | 1.877 | 74.127 | −0.924 | 1.00 | 68.05 | N |
| ATOM | 506 | CA | PHE | A | 100 | 1.723 | 73.626 | 0.436 | 1.00 | 67.53 | C |
| ATOM | 507 | CB | PHE | A | 100 | 2.873 | 72.738 | 0.871 | 1.00 | 68.29 | C |
| ATOM | 508 | CG | PHE | A | 100 | 2.530 | 71.844 | 2.047 | 1.00 | 69.62 | C |
| ATOM | 509 | CD1 | PHE | A | 100 | 1.249 | 71.278 | 2.168 | 1.00 | 70.09 | C |
| ATOM | 510 | CE1 | PHE | A | 100 | 0.933 | 70.435 | 3.245 | 1.00 | 70.04 | C |
| ATOM | 511 | CZ | PHE | A | 100 | 1.906 | 70.147 | 4.214 | 1.00 | 69.68 | C |
| ATOM | 512 | CE2 | PHE | A | 100 | 3.181 | 70.698 | 4.103 | 1.00 | 69.67 | C |
| ATOM | 513 | CD2 | PHE | A | 100 | 3.488 | 71.552 | 3.025 | 1.00 | 70.39 | C |
| ATOM | 514 | C | PHE | A | 100 | 1.617 | 74.720 | 1.453 | 1.00 | 66.54 | C |
| ATOM | 515 | O | PHE | A | 100 | 1.946 | 75.873 | 1.193 | 1.00 | 68.10 | O |
| ATOM | 516 | N | SER | A | 101 | 1.174 | 74.341 | 2.637 | 1.00 | 64.56 | N |
| ATOM | 517 | CA | SER | A | 101 | 0.954 | 75.295 | 3.693 | 1.00 | 61.98 | C |
| ATOM | 518 | CB | SER | A | 101 | −0.524 | 75.693 | 3.717 | 1.00 | 62.34 | C |
| ATOM | 519 | OG | SER | A | 101 | −1.344 | 74.533 | 3.712 | 1.00 | 64.11 | O |
| ATOM | 520 | C | SER | A | 101 | 1.379 | 74.726 | 5.036 | 1.00 | 59.07 | C |
| ATOM | 521 | O | SER | A | 101 | 0.982 | 75.260 | 6.087 | 1.00 | 60.11 | O |
| ATOM | 522 | N | GLY | A | 102 | 2.170 | 73.649 | 5.013 | 1.00 | 55.16 | N |
| ATOM | 523 | CA | GLY | A | 102 | 2.732 | 73.096 | 6.245 | 1.00 | 49.51 | C |
| ATOM | 524 | C | GLY | A | 102 | 3.986 | 73.857 | 6.676 | 1.00 | 46.58 | C |
| ATOM | 525 | O | GLY | A | 102 | 4.576 | 73.592 | 7.726 | 1.00 | 43.90 | O |
| ATOM | 526 | N | VAL | A | 103 | 4.411 | 74.794 | 5.840 | 1.00 | 44.62 | N |
| ATOM | 527 | CA | VAL | A | 103 | 5.521 | 75.673 | 6.162 | 1.00 | 44.58 | C |
| ATOM | 528 | CB | VAL | A | 103 | 6.738 | 75.384 | 5.255 | 1.00 | 44.89 | C |
| ATOM | 529 | CG1 | VAL | A | 103 | 7.832 | 76.366 | 5.505 | 1.00 | 46.40 | C |
| ATOM | 530 | CG2 | VAL | A | 103 | 7.282 | 73.964 | 5.503 | 1.00 | 45.56 | C |
| ATOM | 531 | C | VAL | A | 103 | 5.057 | 77.124 | 6.013 | 1.00 | 43.92 | C |
| ATOM | 532 | O | VAL | A | 103 | 4.370 | 77.458 | 5.049 | 1.00 | 43.86 | O |
| ATOM | 533 | N | ILE | A | 104 | 5.427 | 77.982 | 6.961 | 1.00 | 43.35 | N |
| ATOM | 534 | CA | ILE | A | 104 | 5.168 | 79.411 | 6.824 | 1.00 | 42.66 | C |
| ATOM | 535 | CB | ILE | A | 104 | 5.520 | 80.165 | 8.122 | 1.00 | 43.40 | C |
| ATOM | 536 | CG1 | ILE | A | 104 | 4.339 | 80.057 | 9.077 | 1.00 | 44.47 | C |
| ATOM | 537 | CD1 | ILE | A | 104 | 4.527 | 80.787 | 10.332 | 1.00 | 50.46 | C |
| ATOM | 538 | CG2 | ILE | A | 104 | 5.877 | 81.660 | 7.853 | 1.00 | 41.37 | C |
| ATOM | 539 | C | ILE | A | 104 | 5.961 | 79.925 | 5.622 | 1.00 | 42.85 | C |
| ATOM | 540 | O | ILE | A | 104 | 7.184 | 79.743 | 5.536 | 1.00 | 41.68 | O |
| ATOM | 541 | N | ARG | A | 105 | 5.241 | 80.535 | 4.691 | 1.00 | 42.70 | N |
| ATOM | 542 | CA | ARG | A | 105 | 5.807 | 80.875 | 3.395 | 1.00 | 44.62 | C |
| ATOM | 543 | CB | ARG | A | 105 | 4.690 | 80.815 | 2.360 | 1.00 | 46.35 | C |
| ATOM | 544 | CG | ARG | A | 105 | 5.065 | 81.242 | 0.972 | 1.00 | 53.53 | C |
| ATOM | 545 | CD | ARG | A | 105 | 4.460 | 80.351 | −0.099 | 1.00 | 61.61 | C |
| ATOM | 546 | NE | ARG | A | 105 | 3.071 | 80.019 | 0.185 | 1.00 | 65.80 | N |
| ATOM | 547 | CZ | ARG | A | 105 | 2.156 | 79.818 | −0.763 | 1.00 | 68.07 | C |
| ATOM | 548 | NH1 | ARG | A | 105 | 2.489 | 79.894 | −2.060 | 1.00 | 69.01 | N |
| ATOM | 549 | NH2 | ARG | A | 105 | 0.907 | 79.535 | −0.414 | 1.00 | 68.72 | N |
| ATOM | 550 | C | ARG | A | 105 | 6.464 | 82.255 | 3.412 | 1.00 | 43.35 | C |
| ATOM | 551 | O | ARG | A | 105 | 5.955 | 83.180 | 4.045 | 1.00 | 40.91 | O |
| ATOM | 552 | N | LEU | A | 106 | 7.597 | 82.359 | 2.722 | 1.00 | 42.75 | N |
| ATOM | 553 | CA | LEU | A | 106 | 8.288 | 83.624 | 2.481 | 1.00 | 43.28 | C |
| ATOM | 554 | CB | LEU | A | 106 | 9.746 | 83.349 | 2.126 | 1.00 | 42.30 | C |
| ATOM | 555 | CG | LEU | A | 106 | 10.653 | 84.564 | 1.905 | 1.00 | 42.45 | C |
| ATOM | 556 | CD1 | LEU | A | 106 | 10.906 | 85.341 | 3.204 | 1.00 | 40.95 | C |
| ATOM | 557 | CD2 | LEU | A | 106 | 11.993 | 84.148 | 1.293 | 1.00 | 40.34 | C |
| ATOM | 558 | C | LEU | A | 106 | 7.601 | 84.394 | 1.350 | 1.00 | 44.07 | C |
| ATOM | 559 | O | LEU | A | 106 | 7.620 | 83.960 | 0.206 | 1.00 | 44.25 | O |
| ATOM | 560 | N | LEU | A | 107 | 6.979 | 85.521 | 1.683 | 1.00 | 44.63 | N |
| ATOM | 561 | CA | LEU | A | 107 | 6.241 | 86.316 | 0.712 | 1.00 | 45.98 | C |
| ATOM | 562 | CB | LEU | A | 107 | 5.123 | 87.107 | 1.401 | 1.00 | 46.13 | C |
| ATOM | 563 | CG | LEU | A | 107 | 4.143 | 86.246 | 2.202 | 1.00 | 46.97 | C |
| ATOM | 564 | CD1 | LEU | A | 107 | 3.237 | 87.069 | 3.085 | 1.00 | 47.43 | C |
| ATOM | 565 | CD2 | LEU | A | 107 | 3.330 | 85.376 | 1.239 | 1.00 | 49.67 | C |
| ATOM | 566 | C | LEU | A | 107 | 7.145 | 87.267 | −0.066 | 1.00 | 46.90 | C |
| ATOM | 567 | O | LEU | A | 107 | 6.793 | 87.686 | −1.177 | 1.00 | 46.82 | O |
| ATOM | 568 | N | ASP | A | 108 | 8.302 | 87.601 | 0.511 | 1.00 | 47.11 | N |
| ATOM | 569 | CA | ASP | A | 108 | 9.241 | 88.537 | −0.108 | 1.00 | 47.79 | C |
| ATOM | 570 | CB | ASP | A | 108 | 8.543 | 89.875 | −0.430 | 1.00 | 47.83 | C |
| ATOM | 571 | CG | ASP | A | 108 | 9.311 | 90.725 | −1.464 | 1.00 | 50.15 | C |
| ATOM | 572 | OD1 | ASP | A | 108 | 10.299 | 90.251 | −2.085 | 1.00 | 50.90 | O |
| ATOM | 573 | OD2 | ASP | A | 108 | 8.978 | 91.903 | −1.710 | 1.00 | 52.00 | O |
| ATOM | 574 | C | ASP | A | 108 | 10.392 | 88.791 | 0.841 | 1.00 | 48.02 | C |
| ATOM | 575 | O | ASP | A | 108 | 10.319 | 88.447 | 2.025 | 1.00 | 46.97 | O |
| ATOM | 576 | N | TRP | A | 109 | 11.453 | 89.399 | 0.318 | 1.00 | 48.18 | N |
| ATOM | 577 | CA | TRP | A | 109 | 12.613 | 89.748 | 1.114 | 1.00 | 48.85 | C |
| ATOM | 578 | CB | TRP | A | 109 | 13.598 | 88.588 | 1.170 | 1.00 | 48.93 | C |
| ATOM | 579 | CG | TRP | A | 109 | 14.148 | 88.237 | −0.171 | 1.00 | 51.68 | C |
| ATOM | 580 | CD1 | TRP | A | 109 | 13.543 | 87.478 | −1.137 | 1.00 | 52.64 | C |
| ATOM | 581 | NE1 | TRP | A | 109 | 14.354 | 87.383 | −2.244 | 1.00 | 54.00 | N |
| ATOM | 582 | CE2 | TRP | A | 109 | 15.509 | 88.084 | −2.013 | 1.00 | 54.40 | C |
| ATOM | 583 | CD2 | TRP | A | 109 | 15.407 | 88.645 | −0.715 | 1.00 | 53.99 | C |
| ATOM | 584 | CE3 | TRP | A | 109 | 16.470 | 89.423 | −0.236 | 1.00 | 55.22 | C |
| ATOM | 585 | CZ3 | TRP | A | 109 | 17.577 | 89.625 | −1.056 | 1.00 | 56.91 | C |
| ATOM | 586 | CH2 | TRP | A | 109 | 17.641 | 89.061 | −2.348 | 1.00 | 57.27 | C |
| ATOM | 587 | CZ2 | TRP | A | 109 | 16.621 | 88.288 | −2.839 | 1.00 | 55.31 | C |
| ATOM | 588 | C | TRP | A | 109 | 13.302 | 90.989 | 0.555 | 1.00 | 49.39 | C |
| ATOM | 589 | O | TRP | A | 109 | 13.160 | 91.316 | −0.638 | 1.00 | 49.52 | O |
| ATOM | 590 | N | PHE | A | 110 | 14.043 | 91.672 | 1.425 | 1.00 | 49.52 | N |
| ATOM | 591 | CA | PHE | A | 110 | 14.737 | 92.912 | 1.077 | 1.00 | 50.21 | C |
| ATOM | 592 | CB | PHE | A | 110 | 14.005 | 94.130 | 1.649 | 1.00 | 49.95 | C |
| ATOM | 593 | CG | PHE | A | 110 | 12.583 | 94.259 | 1.182 | 1.00 | 51.89 | C |
| ATOM | 594 | CD1 | PHE | A | 110 | 12.268 | 95.026 | 0.061 | 1.00 | 53.85 | C |
| ATOM | 595 | CE1 | PHE | A | 110 | 10.950 | 95.139 | −0.376 | 1.00 | 54.01 | C |
| ATOM | 596 | CZ | PHE | A | 110 | 9.941 | 94.477 | 0.310 | 1.00 | 54.23 | C |
| ATOM | 597 | CE2 | PHE | A | 110 | 10.249 | 93.710 | 1.426 | 1.00 | 53.04 | C |
| ATOM | 598 | CD2 | PHE | A | 110 | 11.559 | 93.611 | 1.855 | 1.00 | 52.18 | C |
| ATOM | 599 | C | PHE | A | 110 | 16.126 | 92.848 | 1.657 | 1.00 | 50.39 | C |
| ATOM | 600 | O | PHE | A | 110 | 16.331 | 92.251 | 2.711 | 1.00 | 49.25 | O |
| ATOM | 601 | N | GLU | A | 111 | 17.087 | 93.452 | 0.966 | 1.00 | 51.10 | N |
| ATOM | 602 | CA | GLU | A | 111 | 18.440 | 93.541 | 1.494 | 1.00 | 52.22 | C |
| ATOM | 603 | CB | GLU | A | 111 | 19.450 | 93.076 | 0.457 | 1.00 | 52.56 | C |
| ATOM | 604 | CG | GLU | A | 111 | 20.896 | 93.340 | 0.835 | 1.00 | 54.50 | C |
| ATOM | 605 | CD | GLU | A | 111 | 21.857 | 92.662 | −0.109 | 1.00 | 57.61 | C |
| ATOM | 606 | OE1 | GLU | A | 111 | 21.513 | 92.561 | −1.309 | 1.00 | 60.12 | O |
| ATOM | 607 | OE2 | GLU | A | 111 | 22.937 | 92.211 | 0.348 | 1.00 | 59.23 | O |
| ATOM | 608 | C | GLU | A | 111 | 18.751 | 94.974 | 1.938 | 1.00 | 52.56 | C |
| ATOM | 609 | O | GLU | A | 111 | 18.340 | 95.943 | 1.290 | 1.00 | 53.03 | O |
| ATOM | 610 | N | ARG | A | 112 | 19.470 | 95.084 | 3.050 | 1.00 | 52.37 | N |
| ATOM | 611 | CA | ARG | A | 112 | 19.880 | 96.362 | 3.605 | 1.00 | 52.15 | C |
| ATOM | 612 | CB | ARG | A | 112 | 19.204 | 96.601 | 4.957 | 1.00 | 51.55 | C |
| ATOM | 613 | CG | ARG | A | 112 | 17.795 | 97.170 | 4.862 | 1.00 | 50.03 | C |
| ATOM | 614 | CD | ARG | A | 112 | 17.101 | 97.229 | 6.225 | 1.00 | 49.06 | C |
| ATOM | 615 | NE | ARG | A | 112 | 15.825 | 97.939 | 6.172 | 1.00 | 47.63 | N |
| ATOM | 616 | CZ | ARG | A | 112 | 15.036 | 98.135 | 7.223 | 1.00 | 48.41 | C |
| ATOM | 617 | NH1 | ARG | A | 112 | 15.379 | 97.664 | 8.420 | 1.00 | 47.11 | N |
| ATOM | 618 | NH2 | ARG | A | 112 | 13.895 | 98.797 | 7.078 | 1.00 | 48.44 | N |
| ATOM | 619 | C | ARG | A | 112 | 21.380 | 96.312 | 3.789 | 1.00 | 52.90 | C |
| ATOM | 620 | O | ARG | A | 112 | 21.972 | 95.227 | 3.727 | 1.00 | 52.95 | O |
| ATOM | 621 | N | PRO | A | 113 | 22.008 | 97.466 | 4.027 | 1.00 | 53.80 | N |
| ATOM | 622 | CA | PRO | A | 113 | 23.463 | 97.519 | 4.222 | 1.00 | 53.98 | C |
| ATOM | 623 | CB | PRO | A | 113 | 23.696 | 98.958 | 4.718 | 1.00 | 54.67 | C |
| ATOM | 624 | CG | PRO | A | 113 | 22.595 | 99.746 | 4.065 | 1.00 | 54.14 | C |
| ATOM | 625 | CD | PRO | A | 113 | 21.396 | 98.811 | 4.112 | 1.00 | 54.32 | C |
| ATOM | 626 | C | PRO | A | 113 | 23.998 | 96.489 | 5.220 | 1.00 | 54.07 | C |
| ATOM | 627 | O | PRO | A | 113 | 24.980 | 95.812 | 4.914 | 1.00 | 54.49 | O |
| ATOM | 628 | N | ASP | A | 114 | 23.373 | 96.342 | 6.382 | 1.00 | 54.16 | N |
| ATOM | 629 | CA | ASP | A | 114 | 23.903 | 95.378 | 7.346 | 1.00 | 54.11 | C |
| ATOM | 630 | CB | ASP | A | 114 | 24.430 | 96.112 | 8.575 | 1.00 | 55.71 | C |
| ATOM | 631 | CG | ASP | A | 114 | 25.631 | 96.989 | 8.245 | 1.00 | 58.46 | C |
| ATOM | 632 | OD1 | ASP | A | 114 | 25.423 | 98.057 | 7.607 | 1.00 | 61.78 | O |
| ATOM | 633 | OD2 | ASP | A | 114 | 26.805 | 96.681 | 8.573 | 1.00 | 60.03 | O |
| ATOM | 634 | C | ASP | A | 114 | 22.937 | 94.269 | 7.755 | 1.00 | 52.78 | C |
| ATOM | 635 | O | ASP | A | 114 | 23.188 | 93.548 | 8.727 | 1.00 | 53.03 | O |
| ATOM | 636 | N | SER | A | 115 | 21.852 | 94.111 | 6.999 | 1.00 | 50.98 | N |
| ATOM | 637 | CA | SER | A | 115 | 20.856 | 93.105 | 7.331 | 1.00 | 48.41 | C |
| ATOM | 638 | CB | SER | A | 115 | 19.960 | 93.649 | 8.439 | 1.00 | 48.04 | C |
| ATOM | 639 | OG | SER | A | 115 | 18.997 | 94.528 | 7.893 | 1.00 | 45.84 | O |
| ATOM | 640 | C | SER | A | 115 | 19.978 | 92.666 | 6.155 | 1.00 | 47.32 | C |
| ATOM | 641 | O | SER | A | 115 | 19.987 | 93.285 | 5.096 | 1.00 | 46.92 | O |
| ATOM | 642 | N | PHE | A | 116 | 19.198 | 91.609 | 6.381 | 1.00 | 45.66 | N |
| ATOM | 643 | CA | PHE | A | 116 | 18.171 | 91.174 | 5.446 | 1.00 | 44.29 | C |
| ATOM | 644 | CB | PHE | A | 116 | 18.457 | 89.746 | 4.994 | 1.00 | 44.76 | C |
| ATOM | 645 | CG | PHE | A | 116 | 19.567 | 89.632 | 3.980 | 1.00 | 45.24 | C |
| ATOM | 646 | CD1 | PHE | A | 116 | 20.892 | 89.484 | 4.385 | 1.00 | 45.34 | C |
| ATOM | 647 | CE1 | PHE | A | 116 | 21.915 | 89.360 | 3.447 | 1.00 | 47.24 | C |
| ATOM | 648 | CZ | PHE | A | 116 | 21.614 | 89.387 | 2.077 | 1.00 | 46.18 | C |
| ATOM | 649 | CE2 | PHE | A | 116 | 20.291 | 89.531 | 1.661 | 1.00 | 48.08 | C |
| ATOM | 650 | CD2 | PHE | A | 116 | 19.275 | 89.646 | 2.615 | 1.00 | 47.94 | C |
| ATOM | 651 | C | PHE | A | 116 | 16.824 | 91.238 | 6.141 | 1.00 | 43.43 | C |
| ATOM | 652 | O | PHE | A | 116 | 16.721 | 90.945 | 7.333 | 1.00 | 42.72 | O |
| ATOM | 653 | N | VAL | A | 117 | 15.797 | 91.641 | 5.411 | 1.00 | 42.24 | N |
| ATOM | 654 | CA | VAL | A | 117 | 14.450 | 91.681 | 5.945 | 1.00 | 41.74 | C |
| ATOM | 655 | CB | VAL | A | 117 | 13.837 | 93.087 | 5.818 | 1.00 | 41.89 | C |
| ATOM | 656 | CG1 | VAL | A | 117 | 12.473 | 93.136 | 6.447 | 1.00 | 41.40 | C |
| ATOM | 657 | CG2 | VAL | A | 117 | 14.753 | 94.122 | 6.451 | 1.00 | 42.52 | C |
| ATOM | 658 | C | VAL | A | 117 | 13.578 | 90.652 | 5.209 | 1.00 | 41.80 | C |
| ATOM | 659 | O | VAL | A | 117 | 13.507 | 90.660 | 3.974 | 1.00 | 40.77 | O |
| ATOM | 660 | N | LEU | A | 118 | 12.934 | 89.765 | 5.974 | 1.00 | 41.04 | N |
| ATOM | 661 | CA | LEU | A | 118 | 12.094 | 88.693 | 5.410 | 1.00 | 40.22 | C |
| ATOM | 662 | CB | LEU | A | 118 | 12.520 | 87.337 | 5.977 | 1.00 | 39.98 | C |
| ATOM | 663 | CG | LEU | A | 118 | 13.795 | 86.695 | 5.423 | 1.00 | 40.10 | C |
| ATOM | 664 | CD1 | LEU | A | 118 | 15.014 | 87.562 | 5.619 | 1.00 | 42.56 | C |
| ATOM | 665 | CD2 | LEU | A | 118 | 14.032 | 85.325 | 6.063 | 1.00 | 39.40 | C |
| ATOM | 666 | C | LEU | A | 118 | 10.635 | 88.939 | 5.720 | 1.00 | 40.25 | C |
| ATOM | 667 | O | LEU | A | 118 | 10.275 | 89.223 | 6.861 | 1.00 | 39.35 | O |
| ATOM | 668 | N | ILE | A | 119 | 9.795 | 88.842 | 4.700 | 1.00 | 39.45 | N |
| ATOM | 669 | CA | ILE | A | 119 | 8.370 | 89.028 | 4.876 | 1.00 | 40.37 | C |
| ATOM | 670 | CB | ILE | A | 119 | 7.774 | 89.921 | 3.756 | 1.00 | 40.19 | C |
| ATOM | 671 | CG1 | ILE | A | 119 | 8.555 | 91.248 | 3.612 | 1.00 | 41.49 | C |
| ATOM | 672 | CD1 | ILE | A | 119 | 8.540 | 92.131 | 4.855 | 1.00 | 40.16 | C |
| ATOM | 673 | CG2 | ILE | A | 119 | 6.296 | 90.136 | 3.989 | 1.00 | 39.40 | C |
| ATOM | 674 | C | ILE | A | 119 | 7.748 | 87.638 | 4.823 | 1.00 | 41.09 | C |
| ATOM | 675 | O | ILE | A | 119 | 7.793 | 86.966 | 3.788 | 1.00 | 40.59 | O |
| ATOM | 676 | N | LEU | A | 120 | 7.167 | 87.222 | 5.939 | 1.00 | 41.43 | N |
| ATOM | 677 | CA | LEU | A | 120 | 6.634 | 85.872 | 6.076 | 1.00 | 42.78 | C |
| ATOM | 678 | CB | LEU | A | 120 | 7.355 | 85.144 | 7.216 | 1.00 | 41.61 | C |
| ATOM | 679 | CG | LEU | A | 120 | 8.868 | 85.010 | 7.046 | 1.00 | 40.99 | C |
| ATOM | 680 | CD1 | LEU | A | 120 | 9.558 | 84.928 | 8.402 | 1.00 | 43.47 | C |
| ATOM | 681 | CD2 | LEU | A | 120 | 9.234 | 83.785 | 6.187 | 1.00 | 42.48 | C |
| ATOM | 682 | C | LEU | A | 120 | 5.138 | 85.922 | 6.330 | 1.00 | 44.08 | C |
| ATOM | 683 | O | LEU | A | 120 | 4.604 | 86.963 | 6.715 | 1.00 | 44.77 | O |
| ATOM | 684 | N | GLU | A | 121 | 4.449 | 84.808 | 6.109 | 1.00 | 45.28 | N |
| ATOM | 685 | CA | GLU | A | 121 | 3.026 | 84.738 | 6.436 | 1.00 | 47.09 | C |
| ATOM | 686 | CB | GLU | A | 121 | 2.430 | 83.409 | 5.985 | 1.00 | 47.99 | C |
| ATOM | 687 | CG | GLU | A | 121 | 2.534 | 83.123 | 4.497 | 1.00 | 49.97 | C |
| ATOM | 688 | CD | GLU | A | 121 | 1.959 | 81.759 | 4.170 | 1.00 | 53.32 | C |
| ATOM | 689 | OE1 | GLU | A | 121 | 0.911 | 81.714 | 3.506 | 1.00 | 55.61 | O |
| ATOM | 690 | OE2 | GLU | A | 121 | 2.548 | 80.735 | 4.586 | 1.00 | 52.97 | O |
| ATOM | 691 | C | GLU | A | 121 | 2.841 | 84.862 | 7.938 | 1.00 | 47.78 | C |
| ATOM | 692 | O | GLU | A | 121 | 3.753 | 84.550 | 8.711 | 1.00 | 47.35 | O |
| ATOM | 693 | N | ARG | A | 122 | 1.670 | 85.326 | 8.351 | 1.00 | 48.92 | N |
| ATOM | 694 | CA | ARG | A | 122 | 1.369 | 85.439 | 9.766 | 1.00 | 50.98 | C |
| ATOM | 695 | CB | ARG | A | 122 | 1.555 | 86.883 | 10.257 | 1.00 | 50.78 | C |
| ATOM | 696 | CG | ARG | A | 122 | 1.196 | 87.085 | 11.730 | 1.00 | 51.57 | C |
| ATOM | 697 | CD | ARG | A | 122 | 1.716 | 88.383 | 12.349 | 1.00 | 51.63 | C |
| ATOM | 698 | NE | ARG | A | 122 | 1.119 | 89.578 | 11.744 | 1.00 | 52.11 | N |
| ATOM | 699 | CZ | ARG | A | 122 | −0.133 | 89.977 | 11.951 | 1.00 | 51.61 | C |
| ATOM | 700 | NH1 | ARG | A | 122 | −0.937 | 89.274 | 12.741 | 1.00 | 51.42 | N |
| ATOM | 701 | NH2 | ARG | A | 122 | −0.588 | 91.070 | 11.354 | 1.00 | 50.64 | N |
| ATOM | 702 | C | ARG | A | 122 | −0.054 | 84.965 | 10.017 | 1.00 | 52.37 | C |
| ATOM | 703 | O | ARG | A | 122 | −1.005 | 85.749 | 9.922 | 1.00 | 52.94 | O |
| ATOM | 704 | N | PRO | A | 123 | −0.211 | 83.682 | 10.328 | 1.00 | 53.57 | N |
| ATOM | 705 | CA | PRO | A | 123 | −1.529 | 83.141 | 10.672 | 1.00 | 54.00 | C |
| ATOM | 706 | CB | PRO | A | 123 | −1.227 | 81.669 | 10.973 | 1.00 | 54.39 | C |
| ATOM | 707 | CG | PRO | A | 123 | 0.057 | 81.394 | 10.250 | 1.00 | 54.18 | C |
| ATOM | 708 | CD | PRO | A | 123 | 0.845 | 82.652 | 10.398 | 1.00 | 53.87 | C |
| ATOM | 709 | C | PRO | A | 123 | −2.012 | 83.835 | 11.928 | 1.00 | 54.39 | C |
| ATOM | 710 | O | PRO | A | 123 | −1.172 | 84.333 | 12.676 | 1.00 | 54.50 | O |
| ATOM | 711 | N | GLU | A | 124 | −3.322 | 83.862 | 12.164 | 1.00 | 54.85 | N |
| ATOM | 712 | CA | GLU | A | 124 | −3.859 | 84.533 | 13.348 | 1.00 | 55.63 | C |
| ATOM | 713 | CB | GLU | A | 124 | −3.870 | 86.046 | 13.089 | 1.00 | 56.97 | C |
| ATOM | 714 | CG | GLU | A | 124 | −3.856 | 86.946 | 14.335 | 1.00 | 62.93 | C |
| ATOM | 715 | CD | GLU | A | 124 | −4.020 | 88.417 | 13.933 | 1.00 | 69.90 | C |
| ATOM | 716 | OE1 | GLU | A | 124 | −4.962 | 88.742 | 13.153 | 1.00 | 72.13 | O |
| ATOM | 717 | OE2 | GLU | A | 124 | −3.195 | 89.256 | 14.385 | 1.00 | 71.98 | O |
| ATOM | 718 | C | GLU | A | 124 | −5.270 | 84.018 | 13.671 | 1.00 | 53.97 | C |
| ATOM | 719 | O | GLU | A | 124 | −6.093 | 83.910 | 12.764 | 1.00 | 54.52 | O |
| ATOM | 720 | N | PRO | A | 125 | −5.563 | 83.673 | 14.930 | 1.00 | 52.32 | N |
| ATOM | 721 | CA | PRO | A | 125 | −4.601 | 83.673 | 16.040 | 1.00 | 51.14 | C |
| ATOM | 722 | CB | PRO | A | 125 | −5.504 | 83.553 | 17.275 | 1.00 | 51.19 | C |
| ATOM | 723 | CG | PRO | A | 125 | −6.689 | 82.766 | 16.783 | 1.00 | 50.94 | C |
| ATOM | 724 | CD | PRO | A | 125 | −6.906 | 83.255 | 15.374 | 1.00 | 51.87 | C |
| ATOM | 725 | C | PRO | A | 125 | −3.694 | 82.453 | 15.970 | 1.00 | 50.27 | C |
| ATOM | 726 | O | PRO | A | 125 | −4.057 | 81.436 | 15.379 | 1.00 | 49.98 | O |
| ATOM | 727 | N | VAL | A | 126 | −2.526 | 82.562 | 16.588 | 1.00 | 49.17 | N |
| ATOM | 728 | CA | VAL | A | 126 | −1.525 | 81.526 | 16.500 | 1.00 | 48.07 | C |
| ATOM | 729 | CB | VAL | A | 126 | −0.508 | 81.866 | 15.373 | 1.00 | 48.55 | C |
| ATOM | 730 | CG1 | VAL | A | 126 | 0.307 | 83.104 | 15.726 | 1.00 | 49.90 | C |
| ATOM | 731 | CG2 | VAL | A | 126 | 0.400 | 80.711 | 15.096 | 1.00 | 50.31 | C |
| ATOM | 732 | C | VAL | A | 126 | −0.848 | 81.346 | 17.855 | 1.00 | 46.31 | C |
| ATOM | 733 | O | VAL | A | 126 | −0.787 | 82.290 | 18.644 | 1.00 | 46.40 | O |
| ATOM | 734 | N | GLN | A | 127 | −0.360 | 80.129 | 18.117 | 1.00 | 43.89 | N |
| ATOM | 735 | CA | GLN | A | 127 | 0.495 | 79.825 | 19.270 | 1.00 | 41.91 | C |
| ATOM | 736 | CB | GLN | A | 127 | −0.356 | 79.311 | 20.438 | 1.00 | 41.96 | C |
| ATOM | 737 | CG | GLN | A | 127 | 0.414 | 79.085 | 21.751 | 1.00 | 41.25 | C |
| ATOM | 738 | CD | GLN | A | 127 | −0.498 | 78.618 | 22.880 | 1.00 | 41.99 | C |
| ATOM | 739 | OE1 | GLN | A | 127 | −1.346 | 77.744 | 22.688 | 1.00 | 41.61 | O |
| ATOM | 740 | NE2 | GLN | A | 127 | −0.336 | 79.214 | 24.052 | 1.00 | 40.70 | N |
| ATOM | 741 | C | GLN | A | 127 | 1.500 | 78.750 | 18.868 | 1.00 | 41.00 | C |
| ATOM | 742 | O | GLN | A | 127 | 1.136 | 77.807 | 18.153 | 1.00 | 40.30 | O |
| ATOM | 743 | N | ASP | A | 128 | 2.755 | 78.874 | 19.307 | 1.00 | 40.28 | N |
| ATOM | 744 | CA | ASP | A | 128 | 3.719 | 77.829 | 18.995 | 1.00 | 39.77 | C |
| ATOM | 745 | CB | ASP | A | 128 | 5.174 | 78.319 | 19.018 | 1.00 | 40.70 | C |
| ATOM | 746 | CG | ASP | A | 128 | 5.670 | 78.691 | 20.390 | 1.00 | 42.88 | C |
| ATOM | 747 | OD1 | ASP | A | 128 | 5.553 | 77.903 | 21.369 | 1.00 | 46.75 | O |
| ATOM | 748 | OD2 | ASP | A | 128 | 6.231 | 79.788 | 20.562 | 1.00 | 48.33 | O |
| ATOM | 749 | C | ASP | A | 128 | 3.482 | 76.609 | 19.881 | 1.00 | 39.44 | C |
| ATOM | 750 | O | ASP | A | 128 | 2.898 | 76.723 | 20.978 | 1.00 | 38.51 | O |
| ATOM | 751 | N | LEU | A | 129 | 3.908 | 75.443 | 19.392 | 1.00 | 37.35 | N |
| ATOM | 752 | CA | LEU | A | 129 | 3.665 | 74.196 | 20.084 | 1.00 | 35.60 | C |
| ATOM | 753 | CB | LEU | A | 129 | 4.146 | 73.015 | 19.224 | 1.00 | 33.96 | C |
| ATOM | 754 | CG | LEU | A | 129 | 3.950 | 71.607 | 19.773 | 1.00 | 34.42 | C |
| ATOM | 755 | CD1 | LEU | A | 129 | 2.485 | 71.352 | 20.108 | 1.00 | 29.98 | C |
| ATOM | 756 | CD2 | LEU | A | 129 | 4.490 | 70.573 | 18.768 | 1.00 | 33.34 | C |
| ATOM | 757 | C | LEU | A | 129 | 4.281 | 74.165 | 21.489 | 1.00 | 35.69 | C |
| ATOM | 758 | O | LEU | A | 129 | 3.730 | 73.565 | 22.403 | 1.00 | 34.94 | O |
| ATOM | 759 | N | PHE | A | 130 | 5.422 | 74.804 | 21.659 | 1.00 | 36.83 | N |
| ATOM | 760 | CA | PHE | A | 130 | 6.047 | 74.850 | 22.976 | 1.00 | 38.88 | C |
| ATOM | 761 | CB | PHE | A | 130 | 7.342 | 75.665 | 22.930 | 1.00 | 39.30 | C |
| ATOM | 762 | CG | PHE | A | 130 | 8.070 | 75.714 | 24.254 | 1.00 | 42.49 | C |
| ATOM | 763 | CD1 | PHE | A | 130 | 7.678 | 76.621 | 25.251 | 1.00 | 45.52 | C |
| ATOM | 764 | CE1 | PHE | A | 130 | 8.349 | 76.680 | 26.489 | 1.00 | 46.12 | C |
| ATOM | 765 | CZ | PHE | A | 130 | 9.404 | 75.807 | 26.747 | 1.00 | 46.89 | C |
| ATOM | 766 | CE2 | PHE | A | 130 | 9.806 | 74.886 | 25.758 | 1.00 | 47.58 | C |
| ATOM | 767 | CD2 | PHE | A | 130 | 9.132 | 74.851 | 24.514 | 1.00 | 44.28 | C |
| ATOM | 768 | C | PHE | A | 130 | 5.099 | 75.492 | 23.989 | 1.00 | 39.51 | C |
| ATOM | 769 | O | PHE | A | 130 | 4.849 | 74.931 | 25.064 | 1.00 | 38.80 | O |
| ATOM | 770 | N | ASP | A | 131 | 4.596 | 76.679 | 23.657 | 1.00 | 40.69 | N |
| ATOM | 771 | CA | ASP | A | 131 | 3.719 | 77.401 | 24.583 | 1.00 | 42.23 | C |
| ATOM | 772 | CB | ASP | A | 131 | 3.418 | 78.805 | 24.088 | 1.00 | 43.13 | C |
| ATOM | 773 | CG | ASP | A | 131 | 4.620 | 79.699 | 24.113 | 1.00 | 44.46 | C |
| ATOM | 774 | OD1 | ASP | A | 131 | 5.574 | 79.431 | 24.874 | 1.00 | 47.95 | O |
| ATOM | 775 | OD2 | ASP | A | 131 | 4.702 | 80.700 | 23.375 | 1.00 | 49.81 | O |
| ATOM | 776 | C | ASP | A | 131 | 2.433 | 76.642 | 24.771 | 1.00 | 42.09 | C |
| ATOM | 777 | O | ASP | A | 131 | 1.888 | 76.600 | 25.877 | 1.00 | 42.20 | O |
| ATOM | 778 | N | PHE | A | 132 | 1.972 | 76.001 | 23.696 | 1.00 | 41.67 | N |
| ATOM | 779 | CA | PHE | A | 132 | 0.760 | 75.195 | 23.744 | 1.00 | 41.50 | C |
| ATOM | 780 | CB | PHE | A | 132 | 0.459 | 74.630 | 22.358 | 1.00 | 41.82 | C |
| ATOM | 781 | CG | PHE | A | 132 | −0.854 | 73.909 | 22.263 | 1.00 | 40.63 | C |
| ATOM | 782 | CD1 | PHE | A | 132 | −2.039 | 74.618 | 22.148 | 1.00 | 40.87 | C |
| ATOM | 783 | CE1 | PHE | A | 132 | −3.251 | 73.965 | 22.053 | 1.00 | 41.37 | C |
| ATOM | 784 | CZ | PHE | A | 132 | −3.297 | 72.581 | 22.062 | 1.00 | 41.96 | C |
| ATOM | 785 | CE2 | PHE | A | 132 | −2.123 | 71.855 | 22.173 | 1.00 | 40.31 | C |
| ATOM | 786 | CD2 | PHE | A | 132 | −0.910 | 72.524 | 22.281 | 1.00 | 42.21 | C |
| ATOM | 787 | C | PHE | A | 132 | 0.902 | 74.058 | 24.760 | 1.00 | 42.73 | C |
| ATOM | 788 | O | PHE | A | 132 | −0.006 | 73.809 | 25.570 | 1.00 | 42.57 | O |
| ATOM | 789 | N | ILE | A | 133 | 2.040 | 73.369 | 24.718 | 1.00 | 42.81 | N |
| ATOM | 790 | CA | ILE | A | 133 | 2.286 | 72.247 | 25.630 | 1.00 | 43.91 | C |
| ATOM | 791 | CB | ILE | A | 133 | 3.487 | 71.395 | 25.138 | 1.00 | 42.90 | C |
| ATOM | 792 | CG1 | ILE | A | 133 | 3.071 | 70.570 | 23.920 | 1.00 | 41.19 | C |
| ATOM | 793 | CD1 | ILE | A | 133 | 4.220 | 69.961 | 23.174 | 1.00 | 41.52 | C |
| ATOM | 794 | CG2 | ILE | A | 133 | 4.026 | 70.461 | 26.249 | 1.00 | 42.29 | C |
| ATOM | 795 | C | ILE | A | 133 | 2.514 | 72.780 | 27.054 | 1.00 | 46.10 | C |
| ATOM | 796 | O | ILE | A | 133 | 2.046 | 72.191 | 28.023 | 1.00 | 46.11 | O |
| ATOM | 797 | N | THR | A | 134 | 3.231 | 73.894 | 27.162 | 1.00 | 48.42 | N |
| ATOM | 798 | CA | THR | A | 134 | 3.487 | 74.529 | 28.453 | 1.00 | 51.23 | C |
| ATOM | 799 | CB | THR | A | 134 | 4.314 | 75.805 | 28.261 | 1.00 | 51.04 | C |
| ATOM | 800 | OG1 | THR | A | 134 | 5.695 | 75.440 | 28.115 | 1.00 | 52.55 | O |
| ATOM | 801 | CG2 | THR | A | 134 | 4.293 | 76.695 | 29.524 | 1.00 | 53.00 | C |
| ATOM | 802 | C | THR | A | 134 | 2.179 | 74.851 | 29.159 | 1.00 | 52.34 | C |
| ATOM | 803 | O | THR | A | 134 | 2.069 | 74.673 | 30.365 | 1.00 | 53.35 | O |
| ATOM | 804 | N | GLU | A | 135 | 1.188 | 75.303 | 28.400 | 1.00 | 53.42 | N |
| ATOM | 805 | CA | GLU | A | 135 | −0.110 | 75.662 | 28.959 | 1.00 | 54.43 | C |
| ATOM | 806 | CB | GLU | A | 135 | −0.840 | 76.644 | 28.038 | 1.00 | 55.03 | C |
| ATOM | 807 | CG | GLU | A | 135 | −0.137 | 78.004 | 27.941 | 1.00 | 59.01 | C |
| ATOM | 808 | CD | GLU | A | 135 | −0.981 | 79.054 | 27.234 | 1.00 | 62.58 | C |
| ATOM | 809 | OE1 | GLU | A | 135 | −1.942 | 78.685 | 26.505 | 1.00 | 64.20 | O |
| ATOM | 810 | OE2 | GLU | A | 135 | −0.675 | 80.254 | 27.412 | 1.00 | 63.63 | O |
| ATOM | 811 | C | GLU | A | 135 | −1.009 | 74.468 | 29.215 | 1.00 | 53.96 | C |
| ATOM | 812 | O | GLU | A | 135 | −1.743 | 74.440 | 30.206 | 1.00 | 54.61 | O |
| ATOM | 813 | N | ARG | A | 136 | −0.975 | 73.490 | 28.318 | 1.00 | 52.83 | N |
| ATOM | 814 | CA | ARG | A | 136 | −1.947 | 72.404 | 28.385 | 1.00 | 51.61 | C |
| ATOM | 815 | CB | ARG | A | 136 | −2.646 | 72.261 | 27.036 | 1.00 | 52.42 | C |
| ATOM | 816 | CG | ARG | A | 136 | −3.486 | 73.503 | 26.736 | 1.00 | 55.25 | C |
| ATOM | 817 | CD | ARG | A | 136 | −4.130 | 73.538 | 25.378 | 1.00 | 58.99 | C |
| ATOM | 818 | NE | ARG | A | 136 | −4.990 | 72.381 | 25.145 | 1.00 | 60.79 | N |
| ATOM | 819 | CZ | ARG | A | 136 | −6.072 | 72.415 | 24.379 | 1.00 | 61.01 | C |
| ATOM | 820 | NH1 | ARG | A | 136 | −6.425 | 73.559 | 23.777 | 1.00 | 60.57 | N |
| ATOM | 821 | NH2 | ARG | A | 136 | −6.793 | 71.310 | 24.207 | 1.00 | 60.45 | N |
| ATOM | 822 | C | ARG | A | 136 | −1.376 | 71.086 | 28.887 | 1.00 | 50.04 | C |
| ATOM | 823 | O | ARG | A | 136 | −2.116 | 70.144 | 29.119 | 1.00 | 50.37 | O |
| ATOM | 824 | N | GLY | A | 137 | −0.062 | 71.033 | 29.081 | 1.00 | 48.53 | N |
| ATOM | 825 | CA | GLY | A | 137 | 0.597 | 69.799 | 29.477 | 1.00 | 46.78 | C |
| ATOM | 826 | C | GLY | A | 137 | 0.532 | 68.744 | 28.381 | 1.00 | 44.89 | C |
| ATOM | 827 | O | GLY | A | 137 | 0.183 | 69.046 | 27.232 | 1.00 | 45.03 | O |
| ATOM | 828 | N | ALA | A | 138 | 0.849 | 67.509 | 28.748 | 1.00 | 43.07 | N |
| ATOM | 829 | CA | ALA | A | 138 | 0.841 | 66.374 | 27.833 | 1.00 | 41.43 | C |
| ATOM | 830 | CB | ALA | A | 138 | 1.023 | 65.083 | 28.602 | 1.00 | 41.46 | C |
| ATOM | 831 | C | ALA | A | 138 | −0.433 | 66.321 | 26.990 | 1.00 | 40.58 | C |
| ATOM | 832 | O | ALA | A | 138 | −1.533 | 66.476 | 27.491 | 1.00 | 40.85 | O |
| ATOM | 833 | N | LEU | A | 139 | −0.274 | 66.108 | 25.693 | 1.00 | 38.87 | N |
| ATOM | 834 | CA | LEU | A | 139 | −1.415 | 66.107 | 24.794 | 1.00 | 37.16 | C |
| ATOM | 835 | CB | LEU | A | 139 | −0.994 | 66.557 | 23.392 | 1.00 | 34.91 | C |
| ATOM | 836 | CG | LEU | A | 139 | −0.224 | 67.881 | 23.369 | 1.00 | 36.75 | C |
| ATOM | 837 | CD1 | LEU | A | 139 | 0.082 | 68.270 | 21.920 | 1.00 | 35.15 | C |
| ATOM | 838 | CD2 | LEU | A | 139 | −1.002 | 68.999 | 24.124 | 1.00 | 35.61 | C |
| ATOM | 839 | C | LEU | A | 139 | −2.039 | 64.741 | 24.731 | 1.00 | 36.86 | C |
| ATOM | 840 | O | LEU | A | 139 | −1.338 | 63.733 | 24.761 | 1.00 | 37.14 | O |
| ATOM | 841 | N | GLN | A | 140 | −3.362 | 64.714 | 24.626 | 1.00 | 37.15 | N |
| ATOM | 842 | CA | GLN | A | 140 | −4.071 | 63.473 | 24.348 | 1.00 | 38.86 | C |
| ATOM | 843 | CB | GLN | A | 140 | −5.566 | 63.726 | 24.208 | 1.00 | 39.38 | C |
| ATOM | 844 | CG | GLN | A | 140 | −6.266 | 63.885 | 25.540 | 1.00 | 45.52 | C |
| ATOM | 845 | CD | GLN | A | 140 | −7.649 | 64.493 | 25.395 | 1.00 | 52.38 | C |
| ATOM | 846 | OE1 | GLN | A | 140 | −8.442 | 64.070 | 24.534 | 1.00 | 54.06 | O |
| ATOM | 847 | NE2 | GLN | A | 140 | −7.949 | 65.488 | 26.234 | 1.00 | 55.44 | N |
| ATOM | 848 | C | GLN | A | 140 | −3.532 | 62.890 | 23.062 | 1.00 | 37.84 | C |
| ATOM | 849 | O | GLN | A | 140 | −3.192 | 63.643 | 22.141 | 1.00 | 37.63 | O |
| ATOM | 850 | N | GLU | A | 141 | −3.449 | 61.559 | 22.996 | 1.00 | 37.43 | N |
| ATOM | 851 | CA | GLU | A | 141 | −2.897 | 60.881 | 21.808 | 1.00 | 37.42 | C |
| ATOM | 852 | CB | GLU | A | 141 | −2.849 | 59.373 | 22.030 | 1.00 | 37.84 | C |
| ATOM | 853 | CG | GLU | A | 141 | −1.883 | 59.033 | 23.164 | 1.00 | 38.10 | C |
| ATOM | 854 | CD | GLU | A | 141 | −1.571 | 57.568 | 23.263 | 1.00 | 36.74 | C |
| ATOM | 855 | OE1 | GLU | A | 141 | −1.639 | 56.867 | 22.233 | 1.00 | 35.36 | O |
| ATOM | 856 | OE2 | GLU | A | 141 | −1.261 | 57.117 | 24.383 | 1.00 | 37.15 | O |
| ATOM | 857 | C | GLU | A | 141 | −3.596 | 61.227 | 20.498 | 1.00 | 36.95 | C |
| ATOM | 858 | O | GLU | A | 141 | −2.958 | 61.254 | 19.443 | 1.00 | 36.70 | O |
| ATOM | 859 | N | GLU | A | 142 | −4.900 | 61.497 | 20.566 | 1.00 | 36.62 | N |
| ATOM | 860 | CA | GLU | A | 142 | −5.654 | 61.865 | 19.373 | 1.00 | 36.84 | C |
| ATOM | 861 | CB | GLU | A | 142 | −7.151 | 62.019 | 19.677 | 1.00 | 37.69 | C |
| ATOM | 862 | CG | GLU | A | 142 | −7.957 | 62.396 | 18.443 | 1.00 | 39.42 | C |
| ATOM | 863 | CD | GLU | A | 142 | −9.440 | 62.567 | 18.730 | 1.00 | 43.91 | C |
| ATOM | 864 | OE1 | GLU | A | 142 | −9.809 | 63.542 | 19.421 | 1.00 | 44.11 | O |
| ATOM | 865 | OE2 | GLU | A | 142 | −10.233 | 61.727 | 18.254 | 1.00 | 45.16 | O |
| ATOM | 866 | C | GLU | A | 142 | −5.127 | 63.181 | 18.814 | 1.00 | 35.87 | C |
| ATOM | 867 | O | GLU | A | 142 | −4.975 | 63.336 | 17.601 | 1.00 | 35.78 | O |
| ATOM | 868 | N | LEU | A | 143 | −4.857 | 64.120 | 19.709 | 1.00 | 34.68 | N |
| ATOM | 869 | CA | LEU | A | 143 | −4.343 | 65.422 | 19.333 | 1.00 | 33.92 | C |
| ATOM | 870 | CB | LEU | A | 143 | −4.434 | 66.378 | 20.526 | 1.00 | 33.86 | C |
| ATOM | 871 | CG | LEU | A | 143 | −3.933 | 67.812 | 20.341 | 1.00 | 33.72 | C |
| ATOM | 872 | CD1 | LEU | A | 143 | −4.656 | 68.402 | 19.137 | 1.00 | 31.14 | C |
| ATOM | 873 | CD2 | LEU | A | 143 | −4.227 | 68.624 | 21.591 | 1.00 | 34.84 | C |
| ATOM | 874 | C | LEU | A | 143 | −2.898 | 65.304 | 18.842 | 1.00 | 34.15 | C |
| ATOM | 875 | O | LEU | A | 143 | −2.559 | 65.834 | 17.786 | 1.00 | 34.53 | O |
| ATOM | 876 | N | ALA | A | 144 | −2.060 | 64.586 | 19.596 | 1.00 | 33.23 | N |
| ATOM | 877 | CA | ALA | A | 144 | −0.669 | 64.366 | 19.204 | 1.00 | 32.59 | C |
| ATOM | 878 | CB | ALA | A | 144 | 0.046 | 63.540 | 20.247 | 1.00 | 32.52 | C |
| ATOM | 879 | C | ALA | A | 144 | −0.598 | 63.676 | 17.844 | 1.00 | 32.09 | C |
| ATOM | 880 | O | ALA | A | 144 | 0.240 | 64.019 | 17.038 | 1.00 | 31.77 | O |
| ATOM | 881 | N | ARG | A | 145 | −1.494 | 62.720 | 17.587 | 1.00 | 32.32 | N |
| ATOM | 882 | CA | ARG | A | 145 | −1.545 | 62.050 | 16.293 | 1.00 | 32.91 | C |
| ATOM | 883 | CB | ARG | A | 145 | −2.600 | 60.939 | 16.295 | 1.00 | 33.17 | C |
| ATOM | 884 | CG | ARG | A | 145 | −2.769 | 60.208 | 14.961 | 1.00 | 35.72 | C |
| ATOM | 885 | CD | ARG | A | 145 | −3.871 | 59.129 | 14.976 | 1.00 | 37.67 | C |
| ATOM | 886 | NE | ARG | A | 145 | −3.583 | 58.127 | 15.993 | 1.00 | 39.30 | N |
| ATOM | 887 | CZ | ARG | A | 145 | −4.264 | 57.978 | 17.127 | 1.00 | 41.07 | C |
| ATOM | 888 | NH1 | ARG | A | 145 | −5.331 | 58.736 | 17.399 | 1.00 | 41.09 | N |
| ATOM | 889 | NH2 | ARG | A | 145 | −3.884 | 57.050 | 17.987 | 1.00 | 40.07 | N |
| ATOM | 890 | C | ARG | A | 145 | −1.789 | 63.044 | 15.155 | 1.00 | 33.07 | C |
| ATOM | 891 | O | ARG | A | 145 | −1.063 | 63.050 | 14.158 | 1.00 | 32.72 | O |
| ATOM | 892 | N | SER | A | 146 | −2.797 | 63.897 | 15.310 | 1.00 | 33.62 | N |
| ATOM | 893 | CA | SER | A | 146 | −3.103 | 64.896 | 14.287 | 1.00 | 33.98 | C |
| ATOM | 894 | CB | SER | A | 146 | −4.332 | 65.711 | 14.700 | 1.00 | 34.87 | C |
| ATOM | 895 | OG | SER | A | 146 | −4.556 | 66.758 | 13.767 | 1.00 | 37.55 | O |
| ATOM | 896 | C | SER | A | 146 | −1.920 | 65.837 | 14.058 | 1.00 | 34.16 | C |
| ATOM | 897 | O | SER | A | 146 | −1.518 | 66.064 | 12.917 | 1.00 | 34.41 | O |
| ATOM | 898 | N | PHE | A | 147 | −1.349 | 66.347 | 15.154 | 1.00 | 32.41 | N |
| ATOM | 899 | CA | PHE | A | 147 | −0.235 | 67.278 | 15.088 | 1.00 | 32.56 | C |
| ATOM | 900 | CB | PHE | A | 147 | 0.117 | 67.793 | 16.481 | 1.00 | 32.83 | C |
| ATOM | 901 | CG | PHE | A | 147 | −0.765 | 68.925 | 16.972 | 1.00 | 34.64 | C |
| ATOM | 902 | CD1 | PHE | A | 147 | −1.916 | 69.303 | 16.275 | 1.00 | 34.72 | C |
| ATOM | 903 | CE1 | PHE | A | 147 | −2.725 | 70.330 | 16.744 | 1.00 | 37.91 | C |
| ATOM | 904 | CZ | PHE | A | 147 | −2.380 | 71.009 | 17.911 | 1.00 | 36.29 | C |
| ATOM | 905 | CE2 | PHE | A | 147 | −1.223 | 70.642 | 18.617 | 1.00 | 36.31 | C |
| ATOM | 906 | CD2 | PHE | A | 147 | −0.430 | 69.603 | 18.143 | 1.00 | 34.51 | C |
| ATOM | 907 | C | PHE | A | 147 | 1.005 | 66.617 | 14.491 | 1.00 | 32.09 | C |
| ATOM | 908 | O | PHE | A | 147 | 1.647 | 67.190 | 13.625 | 1.00 | 31.07 | O |
| ATOM | 909 | N | PHE | A | 148 | 1.357 | 65.436 | 14.992 | 1.00 | 32.05 | N |
| ATOM | 910 | CA | PHE | A | 148 | 2.516 | 64.706 | 14.486 | 1.00 | 32.29 | C |
| ATOM | 911 | CB | PHE | A | 148 | 2.701 | 63.391 | 15.247 | 1.00 | 32.29 | C |
| ATOM | 912 | CG | PHE | A | 148 | 4.061 | 62.783 | 15.070 | 1.00 | 32.26 | C |
| ATOM | 913 | CD1 | PHE | A | 148 | 5.212 | 63.527 | 15.349 | 1.00 | 32.46 | C |
| ATOM | 914 | CE1 | PHE | A | 148 | 6.477 | 62.979 | 15.206 | 1.00 | 29.15 | C |
| ATOM | 915 | CZ | PHE | A | 148 | 6.610 | 61.665 | 14.771 | 1.00 | 30.20 | C |
| ATOM | 916 | CE2 | PHE | A | 148 | 5.465 | 60.909 | 14.475 | 1.00 | 30.97 | C |
| ATOM | 917 | CD2 | PHE | A | 148 | 4.198 | 61.469 | 14.634 | 1.00 | 31.93 | C |
| ATOM | 918 | C | PHE | A | 148 | 2.385 | 64.405 | 12.990 | 1.00 | 32.02 | C |
| ATOM | 919 | O | PHE | A | 148 | 3.343 | 64.536 | 12.238 | 1.00 | 32.46 | O |
| ATOM | 920 | N | TRP | A | 149 | 1.196 | 64.006 | 12.571 | 1.00 | 31.64 | N |
| ATOM | 921 | CA | TRP | A | 149 | 0.960 | 63.687 | 11.169 | 1.00 | 32.22 | C |
| ATOM | 922 | CB | TRP | A | 149 | −0.469 | 63.221 | 10.973 | 1.00 | 32.32 | C |
| ATOM | 923 | CG | TRP | A | 149 | −0.814 | 62.851 | 9.562 | 1.00 | 33.58 | C |
| ATOM | 924 | CD1 | TRP | A | 149 | −1.276 | 63.695 | 8.583 | 1.00 | 35.97 | C |
| ATOM | 925 | NE1 | TRP | A | 149 | −1.497 | 62.992 | 7.422 | 1.00 | 38.04 | N |
| ATOM | 926 | CE2 | TRP | A | 149 | −1.201 | 61.670 | 7.635 | 1.00 | 35.85 | C |
| ATOM | 927 | CD2 | TRP | A | 149 | −0.773 | 61.542 | 8.978 | 1.00 | 32.84 | C |
| ATOM | 928 | CE3 | TRP | A | 149 | −0.396 | 60.273 | 9.445 | 1.00 | 32.94 | C |
| ATOM | 929 | CZ3 | TRP | A | 149 | −0.480 | 59.181 | 8.574 | 1.00 | 32.70 | C |
| ATOM | 930 | CH2 | TRP | A | 149 | −0.914 | 59.352 | 7.244 | 1.00 | 35.78 | C |
| ATOM | 931 | CZ2 | TRP | A | 149 | −1.279 | 60.584 | 6.763 | 1.00 | 35.70 | C |
| ATOM | 932 | C | TRP | A | 149 | 1.228 | 64.908 | 10.309 | 1.00 | 32.05 | C |
| ATOM | 933 | O | TRP | A | 149 | 1.926 | 64.810 | 9.309 | 1.00 | 31.43 | O |
| ATOM | 934 | N | GLN | A | 150 | 0.720 | 66.078 | 10.729 | 1.00 | 31.85 | N |
| ATOM | 935 | CA | GLN | A | 150 | 0.948 | 67.309 | 9.966 | 1.00 | 31.22 | C |
| ATOM | 936 | CB | GLN | A | 150 | 0.132 | 68.489 | 10.527 | 1.00 | 30.76 | C |
| ATOM | 937 | CG | GLN | A | 150 | −1.376 | 68.335 | 10.336 | 1.00 | 32.09 | C |
| ATOM | 938 | CD | GLN | A | 150 | −2.126 | 69.553 | 10.773 | 1.00 | 34.62 | C |
| ATOM | 939 | OE1 | GLN | A | 150 | −1.850 | 70.656 | 10.292 | 1.00 | 34.95 | O |
| ATOM | 940 | NE2 | GLN | A | 150 | −3.064 | 69.376 | 11.704 | 1.00 | 35.33 | N |
| ATOM | 941 | C | GLN | A | 150 | 2.414 | 67.686 | 9.932 | 1.00 | 31.38 | C |
| ATOM | 942 | O | GLN | A | 150 | 2.884 | 68.278 | 8.942 | 1.00 | 31.25 | O |
| ATOM | 943 | N | VAL | A | 151 | 3.143 | 67.400 | 11.014 | 1.00 | 30.59 | N |
| ATOM | 944 | CA | VAL | A | 151 | 4.576 | 67.691 | 11.006 | 1.00 | 31.25 | C |
| ATOM | 945 | CB | VAL | A | 151 | 5.222 | 67.562 | 12.407 | 1.00 | 31.59 | C |
| ATOM | 946 | CG1 | VAL | A | 151 | 6.736 | 67.703 | 12.328 | 1.00 | 33.21 | C |
| ATOM | 947 | CG2 | VAL | A | 151 | 4.661 | 68.652 | 13.325 | 1.00 | 31.32 | C |
| ATOM | 948 | C | VAL | A | 151 | 5.259 | 66.780 | 9.981 | 1.00 | 30.80 | C |
| ATOM | 949 | O | VAL | A | 151 | 6.140 | 67.215 | 9.239 | 1.00 | 30.84 | O |
| ATOM | 950 | N | LEU | A | 152 | 4.842 | 65.521 | 9.939 | 1.00 | 31.70 | N |
| ATOM | 951 | CA | LEU | A | 152 | 5.429 | 64.564 | 9.000 | 1.00 | 32.14 | C |
| ATOM | 952 | CB | LEU | A | 152 | 4.792 | 63.179 | 9.194 | 1.00 | 32.39 | C |
| ATOM | 953 | CG | LEU | A | 152 | 5.513 | 62.176 | 10.123 | 1.00 | 33.85 | C |
| ATOM | 954 | CD1 | LEU | A | 152 | 6.723 | 61.611 | 9.411 | 1.00 | 35.80 | C |
| ATOM | 955 | CD2 | LEU | A | 152 | 5.950 | 62.799 | 11.422 | 1.00 | 36.98 | C |
| ATOM | 956 | C | LEU | A | 152 | 5.215 | 65.052 | 7.567 | 1.00 | 31.63 | C |
| ATOM | 957 | O | LEU | A | 152 | 6.131 | 65.024 | 6.769 | 1.00 | 32.44 | O |
| ATOM | 958 | N | GLU | A | 153 | 3.997 | 65.471 | 7.252 | 1.00 | 31.63 | N |
| ATOM | 959 | CA | GLU | A | 153 | 3.671 | 65.980 | 5.907 | 1.00 | 32.26 | C |
| ATOM | 960 | CB | GLU | A | 153 | 2.177 | 66.330 | 5.780 | 1.00 | 32.59 | C |
| ATOM | 961 | CG | GLU | A | 153 | 1.233 | 65.126 | 5.736 | 1.00 | 33.60 | C |
| ATOM | 962 | CD | GLU | A | 153 | 1.423 | 64.215 | 4.510 | 1.00 | 35.47 | C |
| ATOM | 963 | OE1 | GLU | A | 153 | 1.617 | 64.717 | 3.392 | 1.00 | 38.01 | O |
| ATOM | 964 | OE2 | GLU | A | 153 | 1.380 | 62.991 | 4.659 | 1.00 | 34.71 | O |
| ATOM | 965 | C | GLU | A | 153 | 4.538 | 67.191 | 5.562 | 1.00 | 31.87 | C |
| ATOM | 966 | O | GLU | A | 153 | 5.076 | 67.281 | 4.449 | 1.00 | 30.83 | O |
| ATOM | 967 | N | ALA | A | 154 | 4.716 | 68.101 | 6.531 | 1.00 | 31.04 | N |
| ATOM | 968 | CA | ALA | A | 154 | 5.548 | 69.291 | 6.318 | 1.00 | 30.48 | C |
| ATOM | 969 | CB | ALA | A | 154 | 5.440 | 70.278 | 7.537 | 1.00 | 31.18 | C |
| ATOM | 970 | C | ALA | A | 154 | 7.002 | 68.933 | 6.082 | 1.00 | 31.22 | C |
| ATOM | 971 | O | ALA | A | 154 | 7.683 | 69.544 | 5.238 | 1.00 | 30.96 | O |
| ATOM | 972 | N | VAL | A | 155 | 7.504 | 67.967 | 6.842 | 1.00 | 31.29 | N |
| ATOM | 973 | CA | VAL | A | 155 | 8.898 | 67.580 | 6.704 | 1.00 | 32.75 | C |
| ATOM | 974 | CB | VAL | A | 155 | 9.335 | 66.651 | 7.856 | 1.00 | 32.61 | C |
| ATOM | 975 | CG1 | VAL | A | 155 | 10.729 | 66.132 | 7.631 | 1.00 | 33.47 | C |
| ATOM | 976 | CG2 | VAL | A | 155 | 9.292 | 67.439 | 9.189 | 1.00 | 35.41 | C |
| ATOM | 977 | C | VAL | A | 155 | 9.094 | 66.905 | 5.336 | 1.00 | 33.10 | C |
| ATOM | 978 | O | VAL | A | 155 | 10.092 | 67.155 | 4.648 | 1.00 | 33.39 | O |
| ATOM | 979 | N | ARG | A | 156 | 8.130 | 66.086 | 4.931 | 1.00 | 33.25 | N |
| ATOM | 980 | CA | ARG | A | 156 | 8.190 | 65.429 | 3.606 | 1.00 | 34.45 | C |
| ATOM | 981 | CB | ARG | A | 156 | 6.992 | 64.490 | 3.396 | 1.00 | 33.21 | C |
| ATOM | 982 | CG | ARG | A | 156 | 6.999 | 63.221 | 4.219 | 1.00 | 33.42 | C |
| ATOM | 983 | CD | ARG | A | 156 | 5.778 | 62.320 | 3.937 | 1.00 | 34.78 | C |
| ATOM | 984 | NE | ARG | A | 156 | 5.644 | 62.064 | 2.494 | 1.00 | 35.47 | N |
| ATOM | 985 | CZ | ARG | A | 156 | 4.533 | 61.636 | 1.903 | 1.00 | 33.43 | C |
| ATOM | 986 | NH1 | ARG | A | 156 | 3.435 | 61.411 | 2.609 | 1.00 | 32.42 | N |
| ATOM | 987 | NH2 | ARG | A | 156 | 4.525 | 61.437 | 0.594 | 1.00 | 34.38 | N |
| ATOM | 988 | C | ARG | A | 156 | 8.211 | 66.491 | 2.501 | 1.00 | 34.92 | C |
| ATOM | 989 | O | ARG | A | 156 | 8.986 | 66.414 | 1.542 | 1.00 | 35.22 | O |
| ATOM | 990 | N | HIS | A | 157 | 7.369 | 67.501 | 2.650 | 1.00 | 36.13 | N |
| ATOM | 991 | CA | HIS | A | 157 | 7.351 | 68.588 | 1.686 | 1.00 | 36.56 | C |
| ATOM | 992 | CB | HIS | A | 157 | 6.299 | 69.629 | 2.048 | 1.00 | 37.38 | C |
| ATOM | 993 | CG | HIS | A | 157 | 6.362 | 70.863 | 1.197 | 1.00 | 39.12 | C |
| ATOM | 994 | ND1 | HIS | A | 157 | 7.005 | 72.014 | 1.608 | 1.00 | 41.07 | N |
| ATOM | 995 | CE1 | HIS | A | 157 | 6.921 | 72.926 | 0.658 | 1.00 | 39.75 | C |
| ATOM | 996 | NE2 | HIS | A | 157 | 6.249 | 72.407 | −0.358 | 1.00 | 40.88 | N |
| ATOM | 997 | CD2 | HIS | A | 157 | 5.873 | 71.124 | −0.041 | 1.00 | 38.81 | C |
| ATOM | 998 | C | HIS | A | 157 | 8.710 | 69.238 | 1.555 | 1.00 | 36.34 | C |
| ATOM | 999 | O | HIS | A | 157 | 9.178 | 69.475 | 0.435 | 1.00 | 36.86 | O |
| ATOM | 1000 | N | CYS | A | 158 | 9.354 | 69.542 | 2.683 | 1.00 | 36.43 | N |
| ATOM | 1001 | CA | CYS | A | 158 | 10.664 | 70.184 | 2.649 | 1.00 | 36.33 | C |
| ATOM | 1002 | CB | CYS | A | 158 | 11.177 | 70.492 | 4.065 | 1.00 | 36.27 | C |
| ATOM | 1003 | SG | CYS | A | 158 | 10.201 | 71.754 | 4.924 | 1.00 | 37.34 | S |
| ATOM | 1004 | C | CYS | A | 158 | 11.663 | 69.290 | 1.937 | 1.00 | 37.14 | C |
| ATOM | 1005 | O | CYS | A | 158 | 12.431 | 69.751 | 1.069 | 1.00 | 36.89 | O |
| ATOM | 1006 | N | HIS | A | 159 | 11.678 | 68.019 | 2.334 | 1.00 | 36.70 | N |
| ATOM | 1007 | CA | HIS | A | 159 | 12.624 | 67.055 | 1.784 | 1.00 | 38.40 | C |
| ATOM | 1008 | CB | HIS | A | 159 | 12.521 | 65.732 | 2.551 | 1.00 | 38.71 | C |
| ATOM | 1009 | CG | HIS | A | 159 | 13.136 | 65.801 | 3.916 | 1.00 | 44.25 | C |
| ATOM | 1010 | ND1 | HIS | A | 159 | 13.788 | 64.734 | 4.499 | 1.00 | 47.72 | N |
| ATOM | 1011 | CE1 | HIS | A | 159 | 14.258 | 65.103 | 5.681 | 1.00 | 49.12 | C |
| ATOM | 1012 | NE2 | HIS | A | 159 | 13.948 | 66.376 | 5.880 | 1.00 | 48.24 | N |
| ATOM | 1013 | CD2 | HIS | A | 159 | 13.238 | 66.834 | 4.798 | 1.00 | 47.23 | C |
| ATOM | 1014 | C | HIS | A | 159 | 12.392 | 66.881 | 0.277 | 1.00 | 38.37 | C |
| ATOM | 1015 | O | HIS | A | 159 | 13.337 | 66.781 | −0.481 | 1.00 | 36.82 | O |
| ATOM | 1016 | N | ASN | A | 160 | 11.128 | 66.926 | −0.127 | 1.00 | 39.53 | N |
| ATOM | 1017 | CA | ASN | A | 160 | 10.742 | 66.927 | −1.529 | 1.00 | 42.14 | C |
| ATOM | 1018 | CB | ASN | A | 160 | 9.239 | 67.045 | −1.629 | 1.00 | 43.88 | C |
| ATOM | 1019 | CG | ASN | A | 160 | 8.602 | 65.778 | −2.013 | 1.00 | 48.90 | C |
| ATOM | 1020 | OD1 | ASN | A | 160 | 8.727 | 64.765 | −1.312 | 1.00 | 53.59 | O |
| ATOM | 1021 | ND2 | ASN | A | 160 | 7.913 | 65.795 | −3.160 | 1.00 | 53.90 | N |
| ATOM | 1022 | C | ASN | A | 160 | 11.322 | 68.100 | −2.286 | 1.00 | 42.07 | C |
| ATOM | 1023 | O | ASN | A | 160 | 11.668 | 67.978 | −3.461 | 1.00 | 41.85 | O |
| ATOM | 1024 | N | CYS | A | 161 | 11.397 | 69.246 | −1.616 | 1.00 | 40.47 | N |
| ATOM | 1025 | CA | CYS | A | 161 | 11.884 | 70.467 | −2.225 | 1.00 | 39.41 | C |
| ATOM | 1026 | CB | CYS | A | 161 | 11.254 | 71.669 | −1.529 | 1.00 | 39.11 | C |
| ATOM | 1027 | SG | CYS | A | 161 | 9.498 | 71.835 | −1.845 | 1.00 | 40.42 | S |
| ATOM | 1028 | C | CYS | A | 161 | 13.391 | 70.551 | −2.129 | 1.00 | 38.90 | C |
| ATOM | 1029 | O | CYS | A | 161 | 13.979 | 71.555 | −2.518 | 1.00 | 39.21 | O |
| ATOM | 1030 | N | GLY | A | 162 | 14.022 | 69.510 | −1.596 | 1.00 | 38.48 | N |
| ATOM | 1031 | CA | GLY | A | 162 | 15.474 | 69.511 | −1.438 | 1.00 | 37.84 | C |
| ATOM | 1032 | C | GLY | A | 162 | 15.957 | 70.269 | −0.221 | 1.00 | 37.83 | C |
| ATOM | 1033 | O | GLY | A | 162 | 17.122 | 70.693 | −0.160 | 1.00 | 36.57 | O |
| ATOM | 1034 | N | VAL | A | 163 | 15.084 | 70.388 | 0.789 | 1.00 | 37.66 | N |
| ATOM | 1035 | CA | VAL | A | 163 | 15.420 | 71.145 | 2.007 | 1.00 | 37.08 | C |
| ATOM | 1036 | CB | VAL | A | 163 | 14.488 | 72.353 | 2.166 | 1.00 | 37.80 | C |
| ATOM | 1037 | CG1 | VAL | A | 163 | 14.748 | 73.082 | 3.511 | 1.00 | 38.56 | C |
| ATOM | 1038 | CG2 | VAL | A | 163 | 14.666 | 73.306 | 1.008 | 1.00 | 36.79 | C |
| ATOM | 1039 | C | VAL | A | 163 | 15.337 | 70.305 | 3.294 | 1.00 | 37.35 | C |
| ATOM | 1040 | O | VAL | A | 163 | 14.363 | 69.589 | 3.538 | 1.00 | 35.09 | O |
| ATOM | 1041 | N | LEU | A | 164 | 16.373 | 70.436 | 4.110 | 1.00 | 37.93 | N |
| ATOM | 1042 | CA | LEU | A | 164 | 16.468 | 69.790 | 5.405 | 1.00 | 38.78 | C |
| ATOM | 1043 | CB | LEU | A | 164 | 17.813 | 69.089 | 5.468 | 1.00 | 39.15 | C |
| ATOM | 1044 | CG | LEU | A | 164 | 18.083 | 68.153 | 6.625 | 1.00 | 41.81 | C |
| ATOM | 1045 | CD1 | LEU | A | 164 | 17.260 | 66.866 | 6.466 | 1.00 | 42.67 | C |
| ATOM | 1046 | CD2 | LEU | A | 164 | 19.553 | 67.843 | 6.711 | 1.00 | 43.27 | C |
| ATOM | 1047 | C | LEU | A | 164 | 16.382 | 70.911 | 6.472 | 1.00 | 38.81 | C |
| ATOM | 1048 | O | LEU | A | 164 | 17.209 | 71.834 | 6.474 | 1.00 | 38.57 | O |
| ATOM | 1049 | N | HIS | A | 165 | 15.387 | 70.830 | 7.357 | 1.00 | 38.45 | N |
| ATOM | 1050 | CA | HIS | A | 165 | 15.164 | 71.860 | 8.388 | 1.00 | 37.77 | C |
| ATOM | 1051 | CB | HIS | A | 165 | 13.758 | 71.731 | 8.991 | 1.00 | 37.59 | C |
| ATOM | 1052 | CG | HIS | A | 165 | 13.398 | 72.836 | 9.937 | 1.00 | 35.86 | C |
| ATOM | 1053 | ND1 | HIS | A | 165 | 13.867 | 72.891 | 11.229 | 1.00 | 33.70 | N |
| ATOM | 1054 | CE1 | HIS | A | 165 | 13.391 | 73.969 | 11.823 | 1.00 | 34.80 | C |
| ATOM | 1055 | NE2 | HIS | A | 165 | 12.628 | 74.617 | 10.959 | 1.00 | 37.29 | N |
| ATOM | 1056 | CD2 | HIS | A | 165 | 12.612 | 73.926 | 9.774 | 1.00 | 35.10 | C |
| ATOM | 1057 | C | HIS | A | 165 | 16.236 | 71.813 | 9.464 | 1.00 | 37.76 | C |
| ATOM | 1058 | O | HIS | A | 165 | 16.784 | 72.843 | 9.824 | 1.00 | 38.35 | O |
| ATOM | 1059 | N | ARG | A | 166 | 16.563 | 70.612 | 9.937 | 1.00 | 37.85 | N |
| ATOM | 1060 | CA | ARG | A | 166 | 17.615 | 70.390 | 10.933 | 1.00 | 38.53 | C |
| ATOM | 1061 | CB | ARG | A | 166 | 18.952 | 70.946 | 10.456 | 1.00 | 39.48 | C |
| ATOM | 1062 | CG | ARG | A | 166 | 19.500 | 70.338 | 9.178 | 1.00 | 42.23 | C |
| ATOM | 1063 | CD | ARG | A | 166 | 20.503 | 71.265 | 8.553 | 1.00 | 46.58 | C |
| ATOM | 1064 | NE | ARG | A | 166 | 21.839 | 70.788 | 8.808 | 1.00 | 50.91 | N |
| ATOM | 1065 | CZ | ARG | A | 166 | 22.933 | 71.523 | 8.743 | 1.00 | 50.31 | C |
| ATOM | 1066 | NH1 | ARG | A | 166 | 22.882 | 72.821 | 8.466 | 1.00 | 50.71 | N |
| ATOM | 1067 | NH2 | ARG | A | 166 | 24.091 | 70.941 | 8.972 | 1.00 | 50.70 | N |
| ATOM | 1068 | C | ARG | A | 166 | 17.370 | 70.951 | 12.331 | 1.00 | 38.67 | C |
| ATOM | 1069 | O | ARG | A | 166 | 18.243 | 70.839 | 13.184 | 1.00 | 39.30 | O |
| ATOM | 1070 | N | ASP | A | 167 | 16.222 | 71.567 | 12.569 | 1.00 | 38.24 | N |
| ATOM | 1071 | CA | ASP | A | 167 | 15.920 | 72.076 | 13.912 | 1.00 | 39.05 | C |
| ATOM | 1072 | CB | ASP | A | 167 | 16.297 | 73.567 | 13.972 | 1.00 | 40.02 | C |
| ATOM | 1073 | CG | ASP | A | 167 | 16.351 | 74.131 | 15.396 | 1.00 | 44.41 | C |
| ATOM | 1074 | OD1 | ASP | A | 167 | 16.656 | 73.391 | 16.374 | 1.00 | 44.09 | O |
| ATOM | 1075 | OD2 | ASP | A | 167 | 16.111 | 75.349 | 15.606 | 1.00 | 47.46 | O |
| ATOM | 1076 | C | ASP | A | 167 | 14.442 | 71.870 | 14.231 | 1.00 | 37.42 | C |
| ATOM | 1077 | O | ASP | A | 167 | 13.765 | 72.783 | 14.722 | 1.00 | 38.05 | O |
| ATOM | 1078 | N | ILE | A | 168 | 13.926 | 70.671 | 13.939 | 1.00 | 36.17 | N |
| ATOM | 1079 | CA | ILE | A | 168 | 12.516 | 70.380 | 14.201 | 1.00 | 34.82 | C |
| ATOM | 1080 | CB | ILE | A | 168 | 12.066 | 69.064 | 13.505 | 1.00 | 35.54 | C |
| ATOM | 1081 | CG1 | ILE | A | 168 | 12.125 | 69.196 | 11.976 | 1.00 | 34.25 | C |
| ATOM | 1082 | CD1 | ILE | A | 168 | 12.194 | 67.836 | 11.258 | 1.00 | 36.78 | C |
| ATOM | 1083 | CG2 | ILE | A | 168 | 10.663 | 68.692 | 13.951 | 1.00 | 34.38 | C |
| ATOM | 1084 | C | ILE | A | 168 | 12.306 | 70.252 | 15.708 | 1.00 | 34.43 | C |
| ATOM | 1085 | O | ILE | A | 168 | 12.914 | 69.409 | 16.350 | 1.00 | 32.59 | O |
| ATOM | 1086 | N | LYS | A | 169 | 11.436 | 71.091 | 16.260 | 1.00 | 33.96 | N |
| ATOM | 1087 | CA | LYS | A | 169 | 11.122 | 71.056 | 17.701 | 1.00 | 33.91 | C |
| ATOM | 1088 | CB | LYS | A | 169 | 12.281 | 71.647 | 18.511 | 1.00 | 34.23 | C |
| ATOM | 1089 | CG | LYS | A | 169 | 12.644 | 73.064 | 18.140 | 1.00 | 35.79 | C |
| ATOM | 1090 | CD | LYS | A | 169 | 13.822 | 73.538 | 18.954 | 1.00 | 40.57 | C |
| ATOM | 1091 | CE | LYS | A | 169 | 14.137 | 75.024 | 18.631 | 1.00 | 44.31 | C |
| ATOM | 1092 | NZ | LYS | A | 169 | 15.134 | 75.616 | 19.597 | 1.00 | 47.28 | N |
| ATOM | 1093 | C | LYS | A | 169 | 9.862 | 71.860 | 17.947 | 1.00 | 33.20 | C |
| ATOM | 1094 | O | LYS | A | 169 | 9.444 | 72.619 | 17.065 | 1.00 | 32.12 | O |
| ATOM | 1095 | N | ASP | A | 170 | 9.272 | 71.731 | 19.138 | 1.00 | 33.18 | N |
| ATOM | 1096 | CA | ASP | A | 170 | 8.021 | 72.433 | 19.438 | 1.00 | 35.25 | C |
| ATOM | 1097 | CB | ASP | A | 170 | 7.517 | 72.132 | 20.839 | 1.00 | 36.00 | C |
| ATOM | 1098 | CG | ASP | A | 170 | 8.582 | 72.296 | 21.895 | 1.00 | 38.87 | C |
| ATOM | 1099 | OD1 | ASP | A | 170 | 9.700 | 72.820 | 21.626 | 1.00 | 41.81 | O |
| ATOM | 1100 | OD2 | ASP | A | 170 | 8.358 | 71.892 | 23.042 | 1.00 | 42.14 | O |
| ATOM | 1101 | C | ASP | A | 170 | 8.075 | 73.934 | 19.226 | 1.00 | 35.41 | C |
| ATOM | 1102 | O | ASP | A | 170 | 7.118 | 74.510 | 18.717 | 1.00 | 35.26 | O |
| ATOM | 1103 | N | GLU | A | 171 | 9.204 | 74.550 | 19.570 | 1.00 | 36.73 | N |
| ATOM | 1104 | CA | GLU | A | 171 | 9.370 | 76.005 | 19.446 | 1.00 | 38.95 | C |
| ATOM | 1105 | CB | GLU | A | 171 | 10.703 | 76.462 | 20.053 | 1.00 | 40.09 | C |
| ATOM | 1106 | CG | GLU | A | 171 | 10.892 | 76.109 | 21.523 | 1.00 | 46.32 | C |
| ATOM | 1107 | CD | GLU | A | 171 | 12.296 | 76.436 | 22.017 | 1.00 | 53.18 | C |
| ATOM | 1108 | OE1 | GLU | A | 171 | 13.229 | 75.621 | 21.798 | 1.00 | 56.05 | O |
| ATOM | 1109 | OE2 | GLU | A | 171 | 12.474 | 77.511 | 22.636 | 1.00 | 57.82 | O |
| ATOM | 1110 | C | GLU | A | 171 | 9.340 | 76.438 | 17.983 | 1.00 | 38.68 | C |
| ATOM | 1111 | O | GLU | A | 171 | 9.000 | 77.583 | 17.678 | 1.00 | 38.39 | O |
| ATOM | 1112 | N | ASN | A | 172 | 9.716 | 75.531 | 17.080 | 1.00 | 37.88 | N |
| ATOM | 1113 | CA | ASN | A | 172 | 9.752 | 75.848 | 15.653 | 1.00 | 37.39 | C |
| ATOM | 1114 | CB | ASN | A | 172 | 11.022 | 75.288 | 15.019 | 1.00 | 37.23 | C |
| ATOM | 1115 | CG | ASN | A | 172 | 12.270 | 76.063 | 15.433 | 1.00 | 38.63 | C |
| ATOM | 1116 | OD1 | ASN | A | 172 | 12.195 | 77.241 | 15.769 | 1.00 | 38.90 | O |
| ATOM | 1117 | ND2 | ASN | A | 172 | 13.421 | 75.407 | 15.390 | 1.00 | 36.90 | N |
| ATOM | 1118 | C | ASN | A | 172 | 8.519 | 75.353 | 14.917 | 1.00 | 36.59 | C |
| ATOM | 1119 | O | ASN | A | 172 | 8.567 | 75.150 | 13.710 | 1.00 | 36.84 | O |
| ATOM | 1120 | N | ILE | A | 173 | 7.430 | 75.141 | 15.653 | 1.00 | 35.61 | N |
| ATOM | 1121 | CA | ILE | A | 173 | 6.143 | 74.742 | 15.085 | 1.00 | 35.39 | C |
| ATOM | 1122 | CB | ILE | A | 173 | 5.797 | 73.292 | 15.516 | 1.00 | 35.63 | C |
| ATOM | 1123 | CG1 | ILE | A | 173 | 6.798 | 72.283 | 14.897 | 1.00 | 36.07 | C |
| ATOM | 1124 | CD1 | ILE | A | 173 | 6.648 | 70.871 | 15.388 | 1.00 | 33.90 | C |
| ATOM | 1125 | CG2 | ILE | A | 173 | 4.356 | 72.954 | 15.161 | 1.00 | 35.62 | C |
| ATOM | 1126 | C | ILE | A | 173 | 5.023 | 75.691 | 15.548 | 1.00 | 36.51 | C |
| ATOM | 1127 | O | ILE | A | 173 | 4.796 | 75.863 | 16.767 | 1.00 | 35.35 | O |
| ATOM | 1128 | N | LEU | A | 174 | 4.319 | 76.286 | 14.588 | 1.00 | 36.38 | N |
| ATOM | 1129 | CA | LEU | A | 174 | 3.223 | 77.192 | 14.900 | 1.00 | 37.97 | C |
| ATOM | 1130 | CB | LEU | A | 174 | 3.307 | 78.506 | 14.107 | 1.00 | 38.37 | C |
| ATOM | 1131 | CG | LEU | A | 174 | 4.444 | 79.472 | 14.437 | 1.00 | 41.65 | C |
| ATOM | 1132 | CD1 | LEU | A | 174 | 4.357 | 80.715 | 13.531 | 1.00 | 44.42 | C |
| ATOM | 1133 | CD2 | LEU | A | 174 | 4.399 | 79.905 | 15.882 | 1.00 | 42.22 | C |
| ATOM | 1134 | C | LEU | A | 174 | 1.891 | 76.518 | 14.642 | 1.00 | 38.02 | C |
| ATOM | 1135 | O | LEU | A | 174 | 1.711 | 75.822 | 13.642 | 1.00 | 37.64 | O |
| ATOM | 1136 | N | ILE | A | 175 | 0.963 | 76.721 | 15.567 | 1.00 | 37.87 | N |
| ATOM | 1137 | CA | ILE | A | 175 | −0.379 | 76.199 | 15.417 | 1.00 | 38.43 | C |
| ATOM | 1138 | CB | ILE | A | 175 | −0.845 | 75.563 | 16.744 | 1.00 | 38.70 | C |
| ATOM | 1139 | CG1 | ILE | A | 175 | 0.148 | 74.510 | 17.228 | 1.00 | 38.66 | C |
| ATOM | 1140 | CD1 | ILE | A | 175 | −0.025 | 74.200 | 18.722 | 1.00 | 41.58 | C |
| ATOM | 1141 | CG2 | ILE | A | 175 | −2.241 | 74.971 | 16.609 | 1.00 | 36.19 | C |
| ATOM | 1142 | C | ILE | A | 175 | −1.342 | 77.313 | 14.997 | 1.00 | 40.30 | C |
| ATOM | 1143 | O | ILE | A | 175 | −1.522 | 78.307 | 15.716 | 1.00 | 41.15 | O |
| ATOM | 1144 | N | ASP | A | 176 | −1.969 | 77.144 | 13.840 | 1.00 | 41.47 | N |
| ATOM | 1145 | CA | ASP | A | 176 | −3.092 | 77.991 | 13.438 | 1.00 | 42.38 | C |
| ATOM | 1146 | CB | ASP | A | 176 | −3.337 | 77.853 | 11.926 | 1.00 | 42.29 | C |
| ATOM | 1147 | CG | ASP | A | 176 | −4.437 | 78.782 | 11.401 | 1.00 | 44.69 | C |
| ATOM | 1148 | OD1 | ASP | A | 176 | −5.440 | 79.033 | 12.113 | 1.00 | 46.71 | O |
| ATOM | 1149 | OD2 | ASP | A | 176 | −4.382 | 79.271 | 10.250 | 1.00 | 43.65 | O |
| ATOM | 1150 | C | ASP | A | 176 | −4.279 | 77.497 | 14.235 | 1.00 | 43.24 | C |
| ATOM | 1151 | O | ASP | A | 176 | −4.904 | 76.483 | 13.882 | 1.00 | 42.40 | O |
| ATOM | 1152 | N | LEU | A | 177 | −4.582 | 78.214 | 15.319 | 1.00 | 44.51 | N |
| ATOM | 1153 | CA | LEU | A | 177 | −5.612 | 77.803 | 16.281 | 1.00 | 45.60 | C |
| ATOM | 1154 | CB | LEU | A | 177 | −5.611 | 78.719 | 17.518 | 1.00 | 45.31 | C |
| ATOM | 1155 | CG | LEU | A | 177 | −4.338 | 78.689 | 18.362 | 1.00 | 45.46 | C |
| ATOM | 1156 | CD1 | LEU | A | 177 | −4.275 | 79.850 | 19.374 | 1.00 | 44.16 | C |
| ATOM | 1157 | CD2 | LEU | A | 177 | −4.247 | 77.335 | 19.066 | 1.00 | 44.99 | C |
| ATOM | 1158 | C | LEU | A | 177 | −7.019 | 77.691 | 15.708 | 1.00 | 46.83 | C |
| ATOM | 1159 | O | LEU | A | 177 | −7.793 | 76.840 | 16.145 | 1.00 | 47.74 | O |
| ATOM | 1160 | N | ASN | A | 178 | −7.348 | 78.535 | 14.737 | 1.00 | 47.91 | N |
| ATOM | 1161 | CA | ASN | A | 178 | −8.664 | 78.512 | 14.104 | 1.00 | 48.63 | C |
| ATOM | 1162 | CB | ASN | A | 178 | −8.886 | 79.810 | 13.316 | 1.00 | 49.80 | C |
| ATOM | 1163 | CG | ASN | A | 178 | −9.487 | 80.939 | 14.169 | 1.00 | 52.87 | C |
| ATOM | 1164 | OD1 | ASN | A | 178 | −9.966 | 80.712 | 15.287 | 1.00 | 55.06 | O |
| ATOM | 1165 | ND2 | ASN | A | 178 | −9.463 | 82.166 | 13.628 | 1.00 | 54.84 | N |
| ATOM | 1166 | C | ASN | A | 178 | −8.843 | 77.332 | 13.154 | 1.00 | 48.41 | C |
| ATOM | 1167 | O | ASN | A | 178 | −9.892 | 76.686 | 13.132 | 1.00 | 49.39 | O |
| ATOM | 1168 | N | ARG | A | 179 | −7.821 | 77.061 | 12.348 | 1.00 | 47.30 | N |
| ATOM | 1169 | CA | ARG | A | 179 | −7.907 | 75.998 | 11.353 | 1.00 | 45.90 | C |
| ATOM | 1170 | CB | ARG | A | 179 | −7.183 | 76.420 | 10.088 | 1.00 | 46.22 | C |
| ATOM | 1171 | CG | ARG | A | 179 | −7.790 | 77.611 | 9.403 | 1.00 | 47.89 | C |
| ATOM | 1172 | CD | ARG | A | 179 | −7.036 | 77.967 | 8.138 | 1.00 | 50.30 | C |
| ATOM | 1173 | NE | ARG | A | 179 | −7.672 | 79.037 | 7.378 | 1.00 | 54.18 | N |
| ATOM | 1174 | CZ | ARG | A | 179 | −8.825 | 78.919 | 6.715 | 1.00 | 56.15 | C |
| ATOM | 1175 | NH1 | ARG | A | 179 | −9.498 | 77.773 | 6.717 | 1.00 | 55.19 | N |
| ATOM | 1176 | NH2 | ARG | A | 179 | −9.309 | 79.958 | 6.042 | 1.00 | 57.24 | N |
| ATOM | 1177 | C | ARG | A | 179 | −7.356 | 74.653 | 11.839 | 1.00 | 44.78 | C |
| ATOM | 1178 | O | ARG | A | 179 | −7.604 | 73.614 | 11.208 | 1.00 | 44.31 | O |
| ATOM | 1179 | N | GLY | A | 180 | −6.612 | 74.667 | 12.946 | 1.00 | 42.54 | N |
| ATOM | 1180 | CA | GLY | A | 180 | −6.001 | 73.448 | 13.448 | 1.00 | 41.77 | C |
| ATOM | 1181 | C | GLY | A | 180 | −4.862 | 72.972 | 12.551 | 1.00 | 41.19 | C |
| ATOM | 1182 | O | GLY | A | 180 | −4.609 | 71.776 | 12.440 | 1.00 | 40.99 | O |
| ATOM | 1183 | N | GLU | A | 181 | −4.172 | 73.908 | 11.909 | 1.00 | 39.77 | N |
| ATOM | 1184 | CA | GLU | A | 181 | −3.105 | 73.549 | 10.986 | 1.00 | 39.51 | C |
| ATOM | 1185 | CB | GLU | A | 181 | −3.335 | 74.241 | 9.641 | 1.00 | 38.79 | C |
| ATOM | 1186 | CG | GLU | A | 181 | −4.438 | 73.608 | 8.809 | 1.00 | 39.75 | C |
| ATOM | 1187 | CD | GLU | A | 181 | −4.919 | 74.501 | 7.676 | 1.00 | 40.13 | C |
| ATOM | 1188 | OE1 | GLU | A | 181 | −4.195 | 75.443 | 7.326 | 1.00 | 42.55 | O |
| ATOM | 1189 | OE2 | GLU | A | 181 | −6.018 | 74.264 | 7.151 | 1.00 | 38.74 | O |
| ATOM | 1190 | C | GLU | A | 181 | −1.761 | 73.957 | 11.553 | 1.00 | 39.26 | C |
| ATOM | 1191 | O | GLU | A | 181 | −1.617 | 75.074 | 12.048 | 1.00 | 39.89 | O |
| ATOM | 1192 | N | LEU | A | 182 | −0.783 | 73.051 | 11.482 | 1.00 | 38.44 | N |
| ATOM | 1193 | CA | LEU | A | 182 | 0.567 | 73.323 | 11.966 | 1.00 | 37.99 | C |
| ATOM | 1194 | CB | LEU | A | 182 | 1.201 | 72.066 | 12.588 | 1.00 | 37.43 | C |
| ATOM | 1195 | CG | LEU | A | 182 | 0.947 | 71.895 | 14.094 | 1.00 | 38.02 | C |
| ATOM | 1196 | CD1 | LEU | A | 182 | −0.528 | 71.939 | 14.378 | 1.00 | 39.25 | C |
| ATOM | 1197 | CD2 | LEU | A | 182 | 1.546 | 70.567 | 14.578 | 1.00 | 35.43 | C |
| ATOM | 1198 | C | LEU | A | 182 | 1.448 | 73.854 | 10.857 | 1.00 | 37.83 | C |
| ATOM | 1199 | O | LEU | A | 182 | 1.256 | 73.519 | 9.688 | 1.00 | 37.14 | O |
| ATOM | 1200 | N | LYS | A | 183 | 2.417 | 74.681 | 11.235 | 1.00 | 37.37 | N |
| ATOM | 1201 | CA | LYS | A | 183 | 3.280 | 75.320 | 10.269 | 1.00 | 38.66 | C |
| ATOM | 1202 | CB | LYS | A | 183 | 2.756 | 76.721 | 9.919 | 1.00 | 39.51 | C |
| ATOM | 1203 | CG | LYS | A | 183 | 1.723 | 76.691 | 8.799 | 1.00 | 44.32 | C |
| ATOM | 1204 | CD | LYS | A | 183 | 1.157 | 78.081 | 8.560 | 1.00 | 50.71 | C |
| ATOM | 1205 | CE | LYS | A | 183 | 0.426 | 78.195 | 7.226 | 1.00 | 53.19 | C |
| ATOM | 1206 | NZ | LYS | A | 183 | −0.586 | 77.117 | 6.989 | 1.00 | 52.77 | N |
| ATOM | 1207 | C | LYS | A | 183 | 4.697 | 75.373 | 10.797 | 1.00 | 37.98 | C |
| ATOM | 1208 | O | LYS | A | 183 | 4.954 | 75.805 | 11.923 | 1.00 | 37.88 | O |
| ATOM | 1209 | N | LEU | A | 184 | 5.617 | 74.918 | 9.969 | 1.00 | 37.28 | N |
| ATOM | 1210 | CA | LEU | A | 184 | 7.015 | 74.830 | 10.333 | 1.00 | 37.45 | C |
| ATOM | 1211 | CB | LEU | A | 184 | 7.658 | 73.726 | 9.494 | 1.00 | 38.41 | C |
| ATOM | 1212 | CG | LEU | A | 184 | 9.013 | 73.126 | 9.811 | 1.00 | 42.44 | C |
| ATOM | 1213 | CD1 | LEU | A | 184 | 9.162 | 72.736 | 11.300 | 1.00 | 44.98 | C |
| ATOM | 1214 | CD2 | LEU | A | 184 | 9.156 | 71.900 | 8.895 | 1.00 | 44.78 | C |
| ATOM | 1215 | C | LEU | A | 184 | 7.689 | 76.195 | 10.135 | 1.00 | 37.17 | C |
| ATOM | 1216 | O | LEU | A | 184 | 7.411 | 76.886 | 9.159 | 1.00 | 34.75 | O |
| ATOM | 1217 | N | ILE | A | 185 | 8.546 | 76.590 | 11.085 | 1.00 | 37.08 | N |
| ATOM | 1218 | CA | ILE | A | 185 | 9.233 | 77.882 | 11.007 | 1.00 | 38.22 | C |
| ATOM | 1219 | CB | ILE | A | 185 | 8.589 | 78.967 | 11.964 | 1.00 | 38.24 | C |
| ATOM | 1220 | CG1 | ILE | A | 185 | 8.676 | 78.523 | 13.428 | 1.00 | 38.15 | C |
| ATOM | 1221 | CD1 | ILE | A | 185 | 8.508 | 79.649 | 14.460 | 1.00 | 40.09 | C |
| ATOM | 1222 | CG2 | ILE | A | 185 | 7.180 | 79.280 | 11.555 | 1.00 | 37.83 | C |
| ATOM | 1223 | C | ILE | A | 185 | 10.678 | 77.766 | 11.365 | 1.00 | 38.83 | C |
| ATOM | 1224 | O | ILE | A | 185 | 11.105 | 76.792 | 12.000 | 1.00 | 38.96 | O |
| ATOM | 1225 | N | ASP | A | 186 | 11.419 | 78.807 | 10.980 | 1.00 | 39.40 | N |
| ATOM | 1226 | CA | ASP | A | 186 | 12.822 | 78.985 | 11.315 | 1.00 | 40.27 | C |
| ATOM | 1227 | CB | ASP | A | 186 | 13.046 | 79.073 | 12.830 | 1.00 | 41.48 | C |
| ATOM | 1228 | CG | ASP | A | 186 | 14.441 | 79.582 | 13.178 | 1.00 | 45.31 | C |
| ATOM | 1229 | OD1 | ASP | A | 186 | 15.190 | 79.992 | 12.255 | 1.00 | 47.25 | O |
| ATOM | 1230 | OD2 | ASP | A | 186 | 14.885 | 79.588 | 14.351 | 1.00 | 50.71 | O |
| ATOM | 1231 | C | ASP | A | 186 | 13.803 | 78.013 | 10.648 | 1.00 | 40.90 | C |
| ATOM | 1232 | O | ASP | A | 186 | 14.343 | 77.096 | 11.285 | 1.00 | 40.21 | O |
| ATOM | 1233 | N | PHE | A | 187 | 14.087 | 78.292 | 9.378 | 1.00 | 40.98 | N |
| ATOM | 1234 | CA | PHE | A | 187 | 15.042 | 77.522 | 8.591 | 1.00 | 42.42 | C |
| ATOM | 1235 | CB | PHE | A | 187 | 14.602 | 77.517 | 7.140 | 1.00 | 41.27 | C |
| ATOM | 1236 | CG | PHE | A | 187 | 13.394 | 76.662 | 6.891 | 1.00 | 41.11 | C |
| ATOM | 1237 | CD1 | PHE | A | 187 | 12.129 | 77.128 | 7.202 | 1.00 | 40.65 | C |
| ATOM | 1238 | CE1 | PHE | A | 187 | 11.000 | 76.342 | 6.977 | 1.00 | 39.93 | C |
| ATOM | 1239 | CZ | PHE | A | 187 | 11.131 | 75.078 | 6.444 | 1.00 | 40.45 | C |
| ATOM | 1240 | CE2 | PHE | A | 187 | 12.398 | 74.586 | 6.128 | 1.00 | 38.34 | C |
| ATOM | 1241 | CD2 | PHE | A | 187 | 13.522 | 75.373 | 6.349 | 1.00 | 40.39 | C |
| ATOM | 1242 | C | PHE | A | 187 | 16.476 | 78.031 | 8.711 | 1.00 | 43.76 | C |
| ATOM | 1243 | O | PHE | A | 187 | 17.346 | 77.647 | 7.927 | 1.00 | 44.80 | O |
| ATOM | 1244 | N | GLY | A | 188 | 16.723 | 78.868 | 9.716 | 1.00 | 44.55 | N |
| ATOM | 1245 | CA | GLY | A | 188 | 18.034 | 79.449 | 9.940 | 1.00 | 45.36 | C |
| ATOM | 1246 | C | GLY | A | 188 | 19.156 | 78.493 | 10.252 | 1.00 | 45.97 | C |
| ATOM | 1247 | O | GLY | A | 188 | 20.320 | 78.870 | 10.168 | 1.00 | 46.90 | O |
| ATOM | 1248 | N | SER | A | 189 | 18.830 | 77.260 | 10.631 | 1.00 | 46.13 | N |
| ATOM | 1249 | CA | SER | A | 189 | 19.853 | 76.240 | 10.866 | 1.00 | 45.91 | C |
| ATOM | 1250 | CB | SER | A | 189 | 19.725 | 75.652 | 12.280 | 1.00 | 46.57 | C |
| ATOM | 1251 | OG | SER | A | 189 | 19.539 | 76.674 | 13.258 | 1.00 | 51.43 | O |
| ATOM | 1252 | C | SER | A | 189 | 19.742 | 75.111 | 9.825 | 1.00 | 44.92 | C |
| ATOM | 1253 | O | SER | A | 189 | 20.356 | 74.051 | 9.977 | 1.00 | 43.79 | O |
| ATOM | 1254 | N | GLY | A | 190 | 18.948 | 75.337 | 8.784 | 1.00 | 44.05 | N |
| ATOM | 1255 | CA | GLY | A | 190 | 18.720 | 74.310 | 7.784 | 1.00 | 43.67 | C |
| ATOM | 1256 | C | GLY | A | 190 | 19.851 | 74.120 | 6.783 | 1.00 | 43.35 | C |
| ATOM | 1257 | O | GLY | A | 190 | 20.908 | 74.764 | 6.862 | 1.00 | 41.72 | O |
| ATOM | 1258 | N | ALA | A | 191 | 19.614 | 73.222 | 5.825 | 1.00 | 42.84 | N |
| ATOM | 1259 | CA | ALA | A | 191 | 20.584 | 72.942 | 4.769 | 1.00 | 41.99 | C |
| ATOM | 1260 | CB | ALA | A | 191 | 21.722 | 72.067 | 5.294 | 1.00 | 41.84 | C |
| ATOM | 1261 | C | ALA | A | 191 | 19.927 | 72.305 | 3.551 | 1.00 | 42.18 | C |
| ATOM | 1262 | O | ALA | A | 191 | 18.779 | 71.813 | 3.608 | 1.00 | 41.24 | O |
| ATOM | 1263 | N | LEU | A | 192 | 20.637 | 72.347 | 2.428 | 1.00 | 42.32 | N |
| ATOM | 1264 | CA | LEU | A | 192 | 20.170 | 71.649 | 1.236 | 1.00 | 42.24 | C |
| ATOM | 1265 | CB | LEU | A | 192 | 21.059 | 71.977 | 0.031 | 1.00 | 43.21 | C |
| ATOM | 1266 | CG | LEU | A | 192 | 21.088 | 73.455 | −0.389 | 1.00 | 46.28 | C |
| ATOM | 1267 | CD1 | LEU | A | 192 | 22.271 | 73.763 | −1.328 | 1.00 | 49.62 | C |
| ATOM | 1268 | CD2 | LEU | A | 192 | 19.778 | 73.889 | −1.025 | 1.00 | 46.71 | C |
| ATOM | 1269 | C | LEU | A | 192 | 20.244 | 70.179 | 1.589 | 1.00 | 41.12 | C |
| ATOM | 1270 | O | LEU | A | 192 | 21.187 | 69.742 | 2.270 | 1.00 | 39.72 | O |
| ATOM | 1271 | N | LEU | A | 193 | 19.227 | 69.428 | 1.190 | 1.00 | 41.80 | N |
| ATOM | 1272 | CA | LEU | A | 193 | 19.237 | 67.983 | 1.401 | 1.00 | 43.29 | C |
| ATOM | 1273 | CB | LEU | A | 193 | 17.870 | 67.405 | 1.066 | 1.00 | 43.47 | C |
| ATOM | 1274 | CG | LEU | A | 193 | 17.658 | 65.896 | 1.213 | 1.00 | 45.93 | C |
| ATOM | 1275 | CD1 | LEU | A | 193 | 17.805 | 65.456 | 2.671 | 1.00 | 45.54 | C |
| ATOM | 1276 | CD2 | LEU | A | 193 | 16.279 | 65.518 | 0.652 | 1.00 | 46.41 | C |
| ATOM | 1277 | C | LEU | A | 193 | 20.306 | 67.331 | 0.512 | 1.00 | 43.97 | C |
| ATOM | 1278 | O | LEU | A | 193 | 20.386 | 67.649 | −0.667 | 1.00 | 44.14 | O |
| ATOM | 1279 | N | LYS | A | 194 | 21.110 | 66.434 | 1.084 | 1.00 | 44.53 | N |
| ATOM | 1280 | CA | LYS | A | 194 | 22.106 | 65.663 | 0.340 | 1.00 | 45.14 | C |
| ATOM | 1281 | CB | LYS | A | 194 | 23.500 | 66.291 | 0.450 | 1.00 | 45.29 | C |
| ATOM | 1282 | CG | LYS | A | 194 | 24.054 | 66.305 | 1.860 | 1.00 | 44.80 | C |
| ATOM | 1283 | CD | LYS | A | 194 | 25.300 | 67.144 | 1.961 | 1.00 | 45.08 | C |
| ATOM | 1284 | CE | LYS | A | 194 | 25.991 | 66.854 | 3.284 | 1.00 | 46.87 | C |
| ATOM | 1285 | NZ | LYS | A | 194 | 27.243 | 67.643 | 3.464 | 1.00 | 48.08 | N |
| ATOM | 1286 | C | LYS | A | 194 | 22.134 | 64.272 | 0.920 | 1.00 | 45.51 | C |
| ATOM | 1287 | O | LYS | A | 194 | 21.615 | 64.054 | 2.026 | 1.00 | 45.00 | O |
| ATOM | 1288 | N | ASP | A | 195 | 22.745 | 63.335 | 0.188 | 1.00 | 45.31 | N |
| ATOM | 1289 | CA | ASP | A | 195 | 22.779 | 61.933 | 0.609 | 1.00 | 45.82 | C |
| ATOM | 1290 | CB | ASP | A | 195 | 22.653 | 60.999 | −0.601 | 1.00 | 46.48 | C |
| ATOM | 1291 | CG | ASP | A | 195 | 21.330 | 61.124 | −1.303 | 1.00 | 47.68 | C |
| ATOM | 1292 | OD1 | ASP | A | 195 | 20.279 | 60.858 | −0.678 | 1.00 | 50.02 | O |
| ATOM | 1293 | OD2 | ASP | A | 195 | 21.243 | 61.469 | −2.499 | 1.00 | 49.94 | O |
| ATOM | 1294 | C | ASP | A | 195 | 24.038 | 61.607 | 1.384 | 1.00 | 45.41 | C |
| ATOM | 1295 | O | ASP | A | 195 | 24.161 | 60.519 | 1.950 | 1.00 | 46.43 | O |
| ATOM | 1296 | N | THR | A | 196 | 24.984 | 62.538 | 1.385 | 1.00 | 45.63 | N |
| ATOM | 1297 | CA | THR | A | 196 | 26.259 | 62.371 | 2.083 | 1.00 | 46.00 | C |
| ATOM | 1298 | CB | THR | A | 196 | 27.394 | 63.091 | 1.322 | 1.00 | 45.69 | C |
| ATOM | 1299 | OG1 | THR | A | 196 | 26.951 | 64.387 | 0.899 | 1.00 | 44.12 | O |
| ATOM | 1300 | CG2 | THR | A | 196 | 27.728 | 62.348 | 0.026 | 1.00 | 46.47 | C |
| ATOM | 1301 | C | THR | A | 196 | 26.211 | 62.902 | 3.518 | 1.00 | 46.75 | C |
| ATOM | 1302 | O | THR | A | 196 | 25.283 | 63.616 | 3.886 | 1.00 | 46.70 | O |
| ATOM | 1303 | N | VAL | A | 197 | 27.237 | 62.569 | 4.302 | 1.00 | 47.32 | N |
| ATOM | 1304 | CA | VAL | A | 197 | 27.294 | 62.912 | 5.713 | 1.00 | 48.22 | C |
| ATOM | 1305 | CB | VAL | A | 197 | 28.440 | 62.174 | 6.437 | 1.00 | 48.78 | C |
| ATOM | 1306 | CG1 | VAL | A | 197 | 29.801 | 62.699 | 6.003 | 1.00 | 50.58 | C |
| ATOM | 1307 | CG2 | VAL | A | 197 | 28.282 | 62.289 | 7.956 | 1.00 | 49.66 | C |
| ATOM | 1308 | C | VAL | A | 197 | 27.366 | 64.409 | 5.965 | 1.00 | 48.10 | C |
| ATOM | 1309 | O | VAL | A | 197 | 27.949 | 65.152 | 5.182 | 1.00 | 47.85 | O |
| ATOM | 1310 | N | TYR | A | 198 | 26.717 | 64.842 | 7.046 | 1.00 | 47.69 | N |
| ATOM | 1311 | CA | TYR | A | 198 | 26.810 | 66.212 | 7.531 | 1.00 | 47.39 | C |
| ATOM | 1312 | CB | TYR | A | 198 | 25.437 | 66.722 | 7.984 | 1.00 | 46.27 | C |
| ATOM | 1313 | CG | TYR | A | 198 | 24.412 | 66.951 | 6.891 | 1.00 | 43.05 | C |
| ATOM | 1314 | CD1 | TYR | A | 198 | 23.574 | 65.924 | 6.464 | 1.00 | 40.17 | C |
| ATOM | 1315 | CE1 | TYR | A | 198 | 22.631 | 66.123 | 5.466 | 1.00 | 39.03 | C |
| ATOM | 1316 | CZ | TYR | A | 198 | 22.490 | 67.368 | 4.904 | 1.00 | 38.13 | C |
| ATOM | 1317 | OH | TYR | A | 198 | 21.539 | 67.577 | 3.933 | 1.00 | 36.97 | O |
| ATOM | 1318 | CE2 | TYR | A | 198 | 23.293 | 68.421 | 5.317 | 1.00 | 39.95 | C |
| ATOM | 1319 | CD2 | TYR | A | 198 | 24.256 | 68.204 | 6.312 | 1.00 | 41.73 | C |
| ATOM | 1320 | C | TYR | A | 198 | 27.753 | 66.211 | 8.729 | 1.00 | 48.60 | C |
| ATOM | 1321 | O | TYR | A | 198 | 27.657 | 65.349 | 9.597 | 1.00 | 48.27 | O |
| ATOM | 1322 | N | THR | A | 199 | 28.658 | 67.183 | 8.775 | 1.00 | 50.37 | N |
| ATOM | 1323 | CA | THR | A | 199 | 29.619 | 67.304 | 9.875 | 1.00 | 52.48 | C |
| ATOM | 1324 | CB | THR | A | 199 | 31.079 | 67.267 | 9.364 | 1.00 | 52.38 | C |
| ATOM | 1325 | OG1 | THR | A | 199 | 31.242 | 68.240 | 8.318 | 1.00 | 53.03 | O |
| ATOM | 1326 | CG2 | THR | A | 199 | 31.393 | 65.936 | 8.714 | 1.00 | 53.00 | C |
| ATOM | 1327 | C | THR | A | 199 | 29.409 | 68.606 | 10.636 | 1.00 | 53.92 | C |
| ATOM | 1328 | O | THR | A | 199 | 30.172 | 68.924 | 11.545 | 1.00 | 53.86 | O |
| ATOM | 1329 | N | ASP | A | 200 | 28.381 | 69.359 | 10.253 | 1.00 | 56.06 | N |
| ATOM | 1330 | CA | ASP | A | 200 | 28.005 | 70.568 | 10.977 | 1.00 | 58.11 | C |
| ATOM | 1331 | CB | ASP | A | 200 | 28.067 | 71.798 | 10.062 | 1.00 | 58.64 | C |
| ATOM | 1332 | CG | ASP | A | 200 | 26.971 | 71.802 | 9.017 | 1.00 | 59.95 | C |
| ATOM | 1333 | OD1 | ASP | A | 200 | 26.266 | 72.826 | 8.884 | 1.00 | 61.08 | O |
| ATOM | 1334 | OD2 | ASP | A | 200 | 26.739 | 70.813 | 8.279 | 1.00 | 63.15 | O |
| ATOM | 1335 | C | ASP | A | 200 | 26.602 | 70.424 | 11.539 | 1.00 | 59.05 | C |
| ATOM | 1336 | O | ASP | A | 200 | 25.751 | 69.737 | 10.957 | 1.00 | 58.97 | O |
| ATOM | 1337 | N | PHE | A | 201 | 26.365 | 71.091 | 12.664 | 1.00 | 60.22 | N |
| ATOM | 1338 | CA | PHE | A | 201 | 25.061 | 71.089 | 13.315 | 1.00 | 61.47 | C |
| ATOM | 1339 | CB | PHE | A | 201 | 24.847 | 69.790 | 14.094 | 1.00 | 61.39 | C |
| ATOM | 1340 | CG | PHE | A | 201 | 23.526 | 69.717 | 14.805 | 1.00 | 61.76 | C |
| ATOM | 1341 | CD1 | PHE | A | 201 | 22.342 | 69.550 | 14.085 | 1.00 | 62.43 | C |
| ATOM | 1342 | CE1 | PHE | A | 201 | 21.110 | 69.475 | 14.741 | 1.00 | 62.41 | C |
| ATOM | 1343 | CZ | PHE | A | 201 | 21.064 | 69.560 | 16.131 | 1.00 | 62.12 | C |
| ATOM | 1344 | CE2 | PHE | A | 201 | 22.242 | 69.727 | 16.856 | 1.00 | 61.55 | C |
| ATOM | 1345 | CD2 | PHE | A | 201 | 23.464 | 69.804 | 16.190 | 1.00 | 61.34 | C |
| ATOM | 1346 | C | PHE | A | 201 | 24.957 | 72.286 | 14.245 | 1.00 | 62.42 | C |
| ATOM | 1347 | O | PHE | A | 201 | 25.712 | 72.411 | 15.214 | 1.00 | 62.75 | O |
| ATOM | 1348 | N | ASP | A | 202 | 24.012 | 73.158 | 13.934 | 1.00 | 63.58 | N |
| ATOM | 1349 | CA | ASP | A | 202 | 23.820 | 74.406 | 14.651 | 1.00 | 64.74 | C |
| ATOM | 1350 | CB | ASP | A | 202 | 24.100 | 75.583 | 13.704 | 1.00 | 65.58 | C |
| ATOM | 1351 | CG | ASP | A | 202 | 23.966 | 76.930 | 14.388 | 1.00 | 69.34 | C |
| ATOM | 1352 | OD1 | ASP | A | 202 | 24.626 | 77.141 | 15.440 | 1.00 | 71.91 | O |
| ATOM | 1353 | OD2 | ASP | A | 202 | 23.207 | 77.831 | 13.950 | 1.00 | 72.83 | O |
| ATOM | 1354 | C | ASP | A | 202 | 22.397 | 74.467 | 15.198 | 1.00 | 64.11 | C |
| ATOM | 1355 | O | ASP | A | 202 | 21.920 | 75.524 | 15.600 | 1.00 | 64.30 | O |
| ATOM | 1356 | N | GLY | A | 203 | 21.716 | 73.324 | 15.202 | 1.00 | 63.47 | N |
| ATOM | 1357 | CA | GLY | A | 203 | 20.358 | 73.250 | 15.712 | 1.00 | 62.03 | C |
| ATOM | 1358 | C | GLY | A | 203 | 20.346 | 72.947 | 17.200 | 1.00 | 60.94 | C |
| ATOM | 1359 | O | GLY | A | 203 | 21.392 | 72.972 | 17.854 | 1.00 | 61.08 | O |
| ATOM | 1360 | N | THR | A | 204 | 19.158 | 72.643 | 17.727 | 1.00 | 59.86 | N |
| ATOM | 1361 | CA | THR | A | 204 | 18.975 | 72.364 | 19.158 | 1.00 | 58.03 | C |
| ATOM | 1362 | CB | THR | A | 204 | 17.481 | 72.402 | 19.547 | 1.00 | 57.90 | C |
| ATOM | 1363 | OG1 | THR | A | 204 | 16.900 | 73.630 | 19.090 | 1.00 | 56.77 | O |
| ATOM | 1364 | CG2 | THR | A | 204 | 17.332 | 72.488 | 21.079 | 1.00 | 57.65 | C |
| ATOM | 1365 | C | THR | A | 204 | 19.574 | 71.032 | 19.575 | 1.00 | 57.57 | C |
| ATOM | 1366 | O | THR | A | 204 | 19.196 | 69.966 | 19.047 | 1.00 | 56.94 | O |
| ATOM | 1367 | N | ARG | A | 205 | 20.487 | 71.106 | 20.545 | 1.00 | 56.60 | N |
| ATOM | 1368 | CA | ARG | A | 205 | 21.238 | 69.959 | 21.022 | 1.00 | 56.09 | C |
| ATOM | 1369 | CB | ARG | A | 205 | 22.204 | 70.417 | 22.124 | 1.00 | 56.67 | C |
| ATOM | 1370 | CG | ARG | A | 205 | 22.870 | 69.291 | 22.879 | 1.00 | 59.97 | C |
| ATOM | 1371 | CD | ARG | A | 205 | 24.127 | 69.719 | 23.631 | 1.00 | 63.64 | C |
| ATOM | 1372 | NE | ARG | A | 205 | 25.317 | 69.608 | 22.785 | 1.00 | 64.42 | N |
| ATOM | 1373 | CZ | ARG | A | 205 | 26.049 | 68.501 | 22.667 | 1.00 | 65.48 | C |
| ATOM | 1374 | NH1 | ARG | A | 205 | 25.712 | 67.410 | 23.340 | 1.00 | 65.75 | N |
| ATOM | 1375 | NH2 | ARG | A | 205 | 27.114 | 68.476 | 21.872 | 1.00 | 64.31 | N |
| ATOM | 1376 | C | ARG | A | 205 | 20.360 | 68.784 | 21.503 | 1.00 | 55.41 | C |
| ATOM | 1377 | O | ARG | A | 205 | 20.536 | 67.630 | 21.069 | 1.00 | 55.27 | O |
| ATOM | 1378 | N | VAL | A | 206 | 19.420 | 69.077 | 22.400 | 1.00 | 54.06 | N |
| ATOM | 1379 | CA | VAL | A | 206 | 18.634 | 68.037 | 23.067 | 1.00 | 52.40 | C |
| ATOM | 1380 | CB | VAL | A | 206 | 17.704 | 68.640 | 24.178 | 1.00 | 52.71 | C |
| ATOM | 1381 | CG1 | VAL | A | 206 | 18.516 | 69.018 | 25.416 | 1.00 | 50.99 | C |
| ATOM | 1382 | CG2 | VAL | A | 206 | 16.919 | 69.844 | 23.636 | 1.00 | 51.73 | C |
| ATOM | 1383 | C | VAL | A | 206 | 17.799 | 67.291 | 22.048 | 1.00 | 51.99 | C |
| ATOM | 1384 | O | VAL | A | 206 | 17.219 | 66.257 | 22.363 | 1.00 | 52.27 | O |
| ATOM | 1385 | N | TYR | A | 207 | 17.731 | 67.834 | 20.830 | 1.00 | 50.50 | N |
| ATOM | 1386 | CA | TYR | A | 207 | 17.001 | 67.202 | 19.738 | 1.00 | 49.94 | C |
| ATOM | 1387 | CB | TYR | A | 207 | 16.126 | 68.236 | 19.021 | 1.00 | 49.34 | C |
| ATOM | 1388 | CG | TYR | A | 207 | 14.759 | 68.542 | 19.600 | 1.00 | 48.61 | C |
| ATOM | 1389 | CD1 | TYR | A | 207 | 14.604 | 69.438 | 20.679 | 1.00 | 49.28 | C |
| ATOM | 1390 | CE1 | TYR | A | 207 | 13.314 | 69.753 | 21.194 | 1.00 | 48.65 | C |
| ATOM | 1391 | CZ | TYR | A | 207 | 12.182 | 69.164 | 20.590 | 1.00 | 50.59 | C |
| ATOM | 1392 | OH | TYR | A | 207 | 10.901 | 69.447 | 21.042 | 1.00 | 47.38 | O |
| ATOM | 1393 | CE2 | TYR | A | 207 | 12.332 | 68.284 | 19.488 | 1.00 | 48.14 | C |
| ATOM | 1394 | CD2 | TYR | A | 207 | 13.605 | 67.999 | 19.007 | 1.00 | 48.95 | C |
| ATOM | 1395 | C | TYR | A | 207 | 17.982 | 66.571 | 18.718 | 1.00 | 49.22 | C |
| ATOM | 1396 | O | TYR | A | 207 | 17.560 | 66.165 | 17.621 | 1.00 | 48.88 | O |
| ATOM | 1397 | N | SER | A | 208 | 19.269 | 66.529 | 19.085 | 1.00 | 48.13 | N |
| ATOM | 1398 | CA | SER | A | 208 | 20.361 | 66.030 | 18.231 | 1.00 | 47.87 | C |
| ATOM | 1399 | CB | SER | A | 208 | 21.667 | 66.791 | 18.496 | 1.00 | 48.10 | C |
| ATOM | 1400 | OG | SER | A | 208 | 22.280 | 66.316 | 19.688 | 1.00 | 49.78 | O |
| ATOM | 1401 | C | SER | A | 208 | 20.620 | 64.566 | 18.503 | 1.00 | 46.42 | C |
| ATOM | 1402 | O | SER | A | 208 | 20.531 | 64.110 | 19.656 | 1.00 | 46.94 | O |
| ATOM | 1403 | N | PRO | A | 209 | 20.941 | 63.826 | 17.449 | 1.00 | 44.93 | N |
| ATOM | 1404 | CA | PRO | A | 209 | 21.043 | 62.374 | 17.545 | 1.00 | 43.05 | C |
| ATOM | 1405 | CB | PRO | A | 209 | 20.979 | 61.948 | 16.083 | 1.00 | 43.11 | C |
| ATOM | 1406 | CG | PRO | A | 209 | 21.596 | 63.062 | 15.366 | 1.00 | 43.72 | C |
| ATOM | 1407 | CD | PRO | A | 209 | 21.165 | 64.293 | 16.070 | 1.00 | 45.04 | C |
| ATOM | 1408 | C | PRO | A | 209 | 22.334 | 61.918 | 18.200 | 1.00 | 42.08 | C |
| ATOM | 1409 | O | PRO | A | 209 | 23.303 | 62.675 | 18.235 | 1.00 | 40.92 | O |
| ATOM | 1410 | N | PRO | A | 210 | 22.355 | 60.685 | 18.705 | 1.00 | 41.24 | N |
| ATOM | 1411 | CA | PRO | A | 210 | 23.546 | 60.167 | 19.374 | 1.00 | 42.13 | C |
| ATOM | 1412 | CB | PRO | A | 210 | 23.117 | 58.762 | 19.830 | 1.00 | 41.13 | C |
| ATOM | 1413 | CG | PRO | A | 210 | 21.980 | 58.403 | 18.942 | 1.00 | 41.77 | C |
| ATOM | 1414 | CD | PRO | A | 210 | 21.270 | 59.693 | 18.669 | 1.00 | 40.59 | C |
| ATOM | 1415 | C | PRO | A | 210 | 24.768 | 60.119 | 18.442 | 1.00 | 43.19 | C |
| ATOM | 1416 | O | PRO | A | 210 | 25.884 | 60.302 | 18.942 | 1.00 | 42.91 | O |
| ATOM | 1417 | N | GLU | A | 211 | 24.567 | 59.901 | 17.138 | 1.00 | 43.80 | N |
| ATOM | 1418 | CA | GLU | A | 211 | 25.683 | 59.896 | 16.184 | 1.00 | 45.23 | C |
| ATOM | 1419 | CB | GLU | A | 211 | 25.253 | 59.400 | 14.780 | 1.00 | 44.59 | C |
| ATOM | 1420 | CG | GLU | A | 211 | 24.227 | 60.279 | 14.079 | 1.00 | 42.32 | C |
| ATOM | 1421 | CD | GLU | A | 211 | 22.796 | 59.821 | 14.334 | 1.00 | 41.06 | C |
| ATOM | 1422 | OE1 | GLU | A | 211 | 22.529 | 59.217 | 15.394 | 1.00 | 38.90 | O |
| ATOM | 1423 | OE2 | GLU | A | 211 | 21.940 | 60.065 | 13.460 | 1.00 | 38.76 | O |
| ATOM | 1424 | C | GLU | A | 211 | 26.354 | 61.263 | 16.095 | 1.00 | 46.56 | C |
| ATOM | 1425 | O | GLU | A | 211 | 27.563 | 61.353 | 15.883 | 1.00 | 46.98 | O |
| ATOM | 1426 | N | TRP | A | 212 | 25.585 | 62.331 | 16.284 | 1.00 | 48.34 | N |
| ATOM | 1427 | CA | TRP | A | 212 | 26.184 | 63.658 | 16.339 | 1.00 | 50.36 | C |
| ATOM | 1428 | CB | TRP | A | 212 | 25.147 | 64.769 | 16.186 | 1.00 | 50.50 | C |
| ATOM | 1429 | CG | TRP | A | 212 | 25.742 | 66.114 | 16.495 | 1.00 | 52.39 | C |
| ATOM | 1430 | CD1 | TRP | A | 212 | 25.599 | 66.830 | 17.652 | 1.00 | 53.01 | C |
| ATOM | 1431 | NE1 | TRP | A | 212 | 26.318 | 67.999 | 17.579 | 1.00 | 53.68 | N |
| ATOM | 1432 | CE2 | TRP | A | 212 | 26.962 | 68.052 | 16.368 | 1.00 | 53.34 | C |
| ATOM | 1433 | CD2 | TRP | A | 212 | 26.626 | 66.877 | 15.661 | 1.00 | 52.72 | C |
| ATOM | 1434 | CE3 | TRP | A | 212 | 27.159 | 66.692 | 14.373 | 1.00 | 52.54 | C |
| ATOM | 1435 | CZ3 | TRP | A | 212 | 27.992 | 67.675 | 13.842 | 1.00 | 52.69 | C |
| ATOM | 1436 | CH2 | TRP | A | 212 | 28.306 | 68.832 | 14.575 | 1.00 | 52.71 | C |
| ATOM | 1437 | CZ2 | TRP | A | 212 | 27.802 | 69.040 | 15.833 | 1.00 | 53.56 | C |
| ATOM | 1438 | C | TRP | A | 212 | 26.996 | 63.835 | 17.622 | 1.00 | 51.79 | C |
| ATOM | 1439 | O | TRP | A | 212 | 28.118 | 64.342 | 17.588 | 1.00 | 52.13 | O |
| ATOM | 1440 | N | ILE | A | 213 | 26.435 | 63.388 | 18.743 | 1.00 | 53.62 | N |
| ATOM | 1441 | CA | ILE | A | 213 | 27.095 | 63.496 | 20.048 | 1.00 | 55.72 | C |
| ATOM | 1442 | CB | ILE | A | 213 | 26.195 | 62.917 | 21.183 | 1.00 | 55.41 | C |
| ATOM | 1443 | CG1 | ILE | A | 213 | 24.804 | 63.568 | 21.202 | 1.00 | 56.07 | C |
| ATOM | 1444 | CD1 | ILE | A | 213 | 24.816 | 65.083 | 21.258 | 1.00 | 57.43 | C |
| ATOM | 1445 | CG2 | ILE | A | 213 | 26.874 | 63.055 | 22.525 | 1.00 | 56.33 | C |
| ATOM | 1446 | C | ILE | A | 213 | 28.440 | 62.771 | 20.050 | 1.00 | 57.10 | C |
| ATOM | 1447 | O | ILE | A | 213 | 29.461 | 63.335 | 20.447 | 1.00 | 57.22 | O |
| ATOM | 1448 | N | ARG | A | 214 | 28.416 | 61.524 | 19.591 | 1.00 | 58.27 | N |
| ATOM | 1449 | CA | ARG | A | 214 | 29.559 | 60.635 | 19.650 | 1.00 | 59.99 | C |
| ATOM | 1450 | CB | ARG | A | 214 | 29.083 | 59.190 | 19.585 | 1.00 | 60.48 | C |
| ATOM | 1451 | CG | ARG | A | 214 | 28.391 | 58.721 | 20.837 | 1.00 | 64.19 | C |
| ATOM | 1452 | CD | ARG | A | 214 | 28.138 | 57.237 | 20.844 | 1.00 | 68.93 | C |
| ATOM | 1453 | NE | ARG | A | 214 | 29.398 | 56.501 | 20.865 | 1.00 | 73.42 | N |
| ATOM | 1454 | CZ | ARG | A | 214 | 29.499 | 55.185 | 21.015 | 1.00 | 76.10 | C |
| ATOM | 1455 | NH1 | ARG | A | 214 | 28.405 | 54.439 | 21.161 | 1.00 | 76.91 | N |
| ATOM | 1456 | NH2 | ARG | A | 214 | 30.697 | 54.609 | 21.013 | 1.00 | 76.26 | N |
| ATOM | 1457 | C | ARG | A | 214 | 30.579 | 60.864 | 18.546 | 1.00 | 59.96 | C |
| ATOM | 1458 | O | ARG | A | 214 | 31.774 | 60.803 | 18.812 | 1.00 | 60.37 | O |
| ATOM | 1459 | N | TYR | A | 215 | 30.116 | 61.106 | 17.318 | 1.00 | 59.65 | N |
| ATOM | 1460 | CA | TYR | A | 215 | 31.018 | 61.161 | 16.159 | 1.00 | 59.46 | C |
| ATOM | 1461 | CB | TYR | A | 215 | 30.751 | 59.997 | 15.196 | 1.00 | 59.78 | C |
| ATOM | 1462 | CG | TYR | A | 215 | 30.624 | 58.648 | 15.858 | 1.00 | 61.91 | C |
| ATOM | 1463 | CD1 | TYR | A | 215 | 31.657 | 58.120 | 16.639 | 1.00 | 63.80 | C |
| ATOM | 1464 | CE1 | TYR | A | 215 | 31.532 | 56.877 | 17.247 | 1.00 | 64.01 | C |
| ATOM | 1465 | CZ | TYR | A | 215 | 30.370 | 56.151 | 17.070 | 1.00 | 65.13 | C |
| ATOM | 1466 | OH | TYR | A | 215 | 30.228 | 54.910 | 17.657 | 1.00 | 66.40 | O |
| ATOM | 1467 | CE2 | TYR | A | 215 | 29.346 | 56.647 | 16.288 | 1.00 | 64.30 | C |
| ATOM | 1468 | CD2 | TYR | A | 215 | 29.475 | 57.885 | 15.692 | 1.00 | 63.31 | C |
| ATOM | 1469 | C | TYR | A | 215 | 30.984 | 62.453 | 15.364 | 1.00 | 58.84 | C |
| ATOM | 1470 | O | TYR | A | 215 | 31.672 | 62.556 | 14.356 | 1.00 | 58.96 | O |
| ATOM | 1471 | N | HIS | A | 216 | 30.189 | 63.431 | 15.791 | 1.00 | 57.99 | N |
| ATOM | 1472 | CA | HIS | A | 216 | 30.018 | 64.666 | 15.020 | 1.00 | 57.44 | C |
| ATOM | 1473 | CB | HIS | A | 216 | 31.238 | 65.583 | 15.198 | 1.00 | 58.54 | C |
| ATOM | 1474 | CG | HIS | A | 216 | 31.302 | 66.231 | 16.547 | 1.00 | 62.71 | C |
| ATOM | 1475 | ND1 | HIS | A | 216 | 30.780 | 67.486 | 16.793 | 1.00 | 65.61 | N |
| ATOM | 1476 | CE1 | HIS | A | 216 | 30.965 | 67.796 | 18.065 | 1.00 | 67.49 | C |
| ATOM | 1477 | NE2 | HIS | A | 216 | 31.591 | 66.788 | 18.655 | 1.00 | 67.79 | N |
| ATOM | 1478 | CD2 | HIS | A | 216 | 31.808 | 65.793 | 17.730 | 1.00 | 65.96 | C |
| ATOM | 1479 | C | HIS | A | 216 | 29.721 | 64.415 | 13.524 | 1.00 | 55.81 | C |
| ATOM | 1480 | O | HIS | A | 216 | 30.212 | 65.135 | 12.653 | 1.00 | 55.97 | O |
| ATOM | 1481 | N | ARG | A | 217 | 28.910 | 63.395 | 13.243 | 1.00 | 53.48 | N |
| ATOM | 1482 | CA | ARG | A | 217 | 28.502 | 63.041 | 11.881 | 1.00 | 51.48 | C |
| ATOM | 1483 | CB | ARG | A | 217 | 29.335 | 61.864 | 11.347 | 1.00 | 51.91 | C |
| ATOM | 1484 | CG | ARG | A | 217 | 30.818 | 62.132 | 11.121 | 1.00 | 54.97 | C |
| ATOM | 1485 | CD | ARG | A | 217 | 31.688 | 60.860 | 11.180 | 1.00 | 59.48 | C |
| ATOM | 1486 | NE | ARG | A | 217 | 31.581 | 60.059 | 9.957 | 1.00 | 63.43 | N |
| ATOM | 1487 | CZ | ARG | A | 217 | 32.061 | 60.411 | 8.751 | 1.00 | 64.73 | C |
| ATOM | 1488 | NH1 | ARG | A | 217 | 32.700 | 61.569 | 8.577 | 1.00 | 66.07 | N |
| ATOM | 1489 | NH2 | ARG | A | 217 | 31.892 | 59.602 | 7.709 | 1.00 | 63.46 | N |
| ATOM | 1490 | C | ARG | A | 217 | 27.054 | 62.581 | 11.923 | 1.00 | 48.94 | C |
| ATOM | 1491 | O | ARG | A | 217 | 26.641 | 61.939 | 12.884 | 1.00 | 48.53 | O |
| ATOM | 1492 | N | TYR | A | 218 | 26.300 | 62.893 | 10.875 | 1.00 | 45.99 | N |
| ATOM | 1493 | CA | TYR | A | 218 | 24.938 | 62.394 | 10.722 | 1.00 | 43.71 | C |
| ATOM | 1494 | CB | TYR | A | 218 | 23.976 | 63.126 | 11.694 | 1.00 | 42.32 | C |
| ATOM | 1495 | CG | TYR | A | 218 | 23.830 | 64.587 | 11.395 | 1.00 | 40.14 | C |
| ATOM | 1496 | CD1 | TYR | A | 218 | 24.708 | 65.529 | 11.937 | 1.00 | 39.79 | C |
| ATOM | 1497 | CE1 | TYR | A | 218 | 24.574 | 66.882 | 11.628 | 1.00 | 42.32 | C |
| ATOM | 1498 | CZ | TYR | A | 218 | 23.562 | 67.298 | 10.770 | 1.00 | 41.14 | C |
| ATOM | 1499 | OH | TYR | A | 218 | 23.412 | 68.630 | 10.464 | 1.00 | 43.32 | O |
| ATOM | 1500 | CE2 | TYR | A | 218 | 22.680 | 66.375 | 10.224 | 1.00 | 39.68 | C |
| ATOM | 1501 | CD2 | TYR | A | 218 | 22.828 | 65.031 | 10.539 | 1.00 | 39.80 | C |
| ATOM | 1502 | C | TYR | A | 218 | 24.448 | 62.520 | 9.279 | 1.00 | 42.58 | C |
| ATOM | 1503 | O | TYR | A | 218 | 24.959 | 63.323 | 8.492 | 1.00 | 42.66 | O |
| ATOM | 1504 | N | HIS | A | 219 | 23.434 | 61.732 | 8.947 | 1.00 | 41.95 | N |
| ATOM | 1505 | CA | HIS | A | 219 | 22.769 | 61.849 | 7.658 | 1.00 | 40.52 | C |
| ATOM | 1506 | CB | HIS | A | 219 | 22.655 | 60.465 | 7.030 | 1.00 | 41.07 | C |
| ATOM | 1507 | CG | HIS | A | 219 | 23.984 | 59.906 | 6.614 | 1.00 | 42.12 | C |
| ATOM | 1508 | ND1 | HIS | A | 219 | 24.497 | 60.080 | 5.344 | 1.00 | 43.99 | N |
| ATOM | 1509 | CE1 | HIS | A | 219 | 25.692 | 59.523 | 5.273 | 1.00 | 42.45 | C |
| ATOM | 1510 | NE2 | HIS | A | 219 | 25.982 | 59.010 | 6.455 | 1.00 | 43.76 | N |
| ATOM | 1511 | CD2 | HIS | A | 219 | 24.935 | 59.246 | 7.317 | 1.00 | 41.90 | C |
| ATOM | 1512 | C | HIS | A | 219 | 21.404 | 62.521 | 7.843 | 1.00 | 39.33 | C |
| ATOM | 1513 | O | HIS | A | 219 | 20.779 | 62.370 | 8.889 | 1.00 | 38.64 | O |
| ATOM | 1514 | N | GLY | A | 220 | 20.965 | 63.270 | 6.836 | 1.00 | 38.11 | N |
| ATOM | 1515 | CA | GLY | A | 220 | 19.784 | 64.103 | 6.950 | 1.00 | 37.81 | C |
| ATOM | 1516 | C | GLY | A | 220 | 18.527 | 63.394 | 7.405 | 1.00 | 38.27 | C |
| ATOM | 1517 | O | GLY | A | 220 | 17.931 | 63.745 | 8.429 | 1.00 | 37.53 | O |
| ATOM | 1518 | N | ARG | A | 221 | 18.122 | 62.386 | 6.647 | 1.00 | 38.11 | N |
| ATOM | 1519 | CA | ARG | A | 221 | 16.855 | 61.717 | 6.895 | 1.00 | 38.50 | C |
| ATOM | 1520 | CB | ARG | A | 221 | 16.542 | 60.729 | 5.767 | 1.00 | 40.47 | C |
| ATOM | 1521 | CG | ARG | A | 221 | 16.585 | 61.401 | 4.388 | 1.00 | 45.37 | C |
| ATOM | 1522 | CD | ARG | A | 221 | 16.575 | 60.448 | 3.185 | 1.00 | 51.09 | C |
| ATOM | 1523 | NE | ARG | A | 221 | 16.584 | 61.200 | 1.919 | 1.00 | 53.82 | N |
| ATOM | 1524 | CZ | ARG | A | 221 | 17.690 | 61.495 | 1.222 | 1.00 | 55.73 | C |
| ATOM | 1525 | NH1 | ARG | A | 221 | 18.894 | 61.099 | 1.646 | 1.00 | 56.00 | N |
| ATOM | 1526 | NH2 | ARG | A | 221 | 17.594 | 62.164 | 0.075 | 1.00 | 55.14 | N |
| ATOM | 1527 | C | ARG | A | 221 | 16.824 | 61.050 | 8.256 | 1.00 | 37.41 | C |
| ATOM | 1528 | O | ARG | A | 221 | 15.873 | 61.254 | 9.013 | 1.00 | 37.16 | O |
| ATOM | 1529 | N | SER | A | 222 | 17.858 | 60.290 | 8.597 | 1.00 | 36.13 | N |
| ATOM | 1530 | CA | SER | A | 222 | 17.836 | 59.563 | 9.863 | 1.00 | 35.73 | C |
| ATOM | 1531 | CB | SER | A | 222 | 18.900 | 58.448 | 9.890 | 1.00 | 34.73 | C |
| ATOM | 1532 | OG | SER | A | 222 | 20.215 | 58.968 | 9.772 | 1.00 | 36.77 | O |
| ATOM | 1533 | C | SER | A | 222 | 17.941 | 60.519 | 11.069 | 1.00 | 35.26 | C |
| ATOM | 1534 | O | SER | A | 222 | 17.365 | 60.250 | 12.137 | 1.00 | 35.32 | O |
| ATOM | 1535 | N | ALA | A | 223 | 18.647 | 61.633 | 10.899 | 1.00 | 34.89 | N |
| ATOM | 1536 | CA | ALA | A | 223 | 18.743 | 62.643 | 11.958 | 1.00 | 34.34 | C |
| ATOM | 1537 | CB | ALA | A | 223 | 19.847 | 63.660 | 11.666 | 1.00 | 32.42 | C |
| ATOM | 1538 | C | ALA | A | 223 | 17.399 | 63.350 | 12.101 | 1.00 | 34.25 | C |
| ATOM | 1539 | O | ALA | A | 223 | 16.992 | 63.726 | 13.214 | 1.00 | 33.94 | O |
| ATOM | 1540 | N | ALA | A | 224 | 16.699 | 63.523 | 10.983 | 1.00 | 33.64 | N |
| ATOM | 1541 | CA | ALA | A | 224 | 15.384 | 64.152 | 11.033 | 1.00 | 32.93 | C |
| ATOM | 1542 | CB | ALA | A | 224 | 14.862 | 64.456 | 9.651 | 1.00 | 33.29 | C |
| ATOM | 1543 | C | ALA | A | 224 | 14.410 | 63.269 | 11.812 | 1.00 | 33.44 | C |
| ATOM | 1544 | O | ALA | A | 224 | 13.645 | 63.770 | 12.651 | 1.00 | 33.58 | O |
| ATOM | 1545 | N | VAL | A | 225 | 14.455 | 61.962 | 11.562 | 1.00 | 32.40 | N |
| ATOM | 1546 | CA | VAL | A | 225 | 13.569 | 61.003 | 12.228 | 1.00 | 31.69 | C |
| ATOM | 1547 | CB | VAL | A | 225 | 13.724 | 59.574 | 11.622 | 1.00 | 32.53 | C |
| ATOM | 1548 | CG1 | VAL | A | 225 | 13.083 | 58.504 | 12.507 | 1.00 | 30.93 | C |
| ATOM | 1549 | CG2 | VAL | A | 225 | 13.123 | 59.544 | 10.219 | 1.00 | 32.75 | C |
| ATOM | 1550 | C | VAL | A | 225 | 13.856 | 60.988 | 13.740 | 1.00 | 32.12 | C |
| ATOM | 1551 | O | VAL | A | 225 | 12.943 | 60.876 | 14.552 | 1.00 | 31.44 | O |
| ATOM | 1552 | N | TRP | A | 226 | 15.125 | 61.117 | 14.110 | 1.00 | 31.95 | N |
| ATOM | 1553 | CA | TRP | A | 226 | 15.476 | 61.173 | 15.530 | 1.00 | 32.47 | C |
| ATOM | 1554 | CB | TRP | A | 226 | 16.990 | 61.279 | 15.721 | 1.00 | 33.06 | C |
| ATOM | 1555 | CG | TRP | A | 226 | 17.322 | 61.494 | 17.183 | 1.00 | 32.56 | C |
| ATOM | 1556 | CD1 | TRP | A | 226 | 17.334 | 62.682 | 17.851 | 1.00 | 32.49 | C |
| ATOM | 1557 | NE1 | TRP | A | 226 | 17.660 | 62.479 | 19.173 | 1.00 | 32.64 | N |
| ATOM | 1558 | CE2 | TRP | A | 226 | 17.834 | 61.134 | 19.383 | 1.00 | 31.73 | C |
| ATOM | 1559 | CD2 | TRP | A | 226 | 17.631 | 60.485 | 18.148 | 1.00 | 31.24 | C |
| ATOM | 1560 | CE3 | TRP | A | 226 | 17.757 | 59.089 | 18.094 | 1.00 | 32.89 | C |
| ATOM | 1561 | CZ3 | TRP | A | 226 | 18.096 | 58.391 | 19.261 | 1.00 | 32.32 | C |
| ATOM | 1562 | CH2 | TRP | A | 226 | 18.286 | 59.080 | 20.478 | 1.00 | 33.34 | C |
| ATOM | 1563 | CZ2 | TRP | A | 226 | 18.157 | 60.444 | 20.552 | 1.00 | 32.43 | C |
| ATOM | 1564 | C | TRP | A | 226 | 14.754 | 62.372 | 16.178 | 1.00 | 32.00 | C |
| ATOM | 1565 | O | TRP | A | 226 | 14.071 | 62.224 | 17.192 | 1.00 | 31.94 | O |
| ATOM | 1566 | N | SER | A | 227 | 14.872 | 63.546 | 15.558 | 1.00 | 31.65 | N |
| ATOM | 1567 | CA | SER | A | 227 | 14.217 | 64.752 | 16.073 | 1.00 | 31.57 | C |
| ATOM | 1568 | CB | SER | A | 227 | 14.611 | 65.982 | 15.259 | 1.00 | 31.61 | C |
| ATOM | 1569 | OG | SER | A | 227 | 13.916 | 66.048 | 14.016 | 1.00 | 33.45 | O |
| ATOM | 1570 | C | SER | A | 227 | 12.695 | 64.599 | 16.161 | 1.00 | 31.31 | C |
| ATOM | 1571 | O | SER | A | 227 | 12.052 | 65.151 | 17.072 | 1.00 | 30.56 | O |
| ATOM | 1572 | N | LEU | A | 228 | 12.124 | 63.841 | 15.229 | 1.00 | 30.36 | N |
| ATOM | 1573 | CA | LEU | A | 228 | 10.701 | 63.545 | 15.217 | 1.00 | 30.48 | C |
| ATOM | 1574 | CB | LEU | A | 228 | 10.300 | 62.865 | 13.901 | 1.00 | 30.69 | C |
| ATOM | 1575 | CG | LEU | A | 228 | 10.325 | 63.767 | 12.661 | 1.00 | 31.45 | C |
| ATOM | 1576 | CD1 | LEU | A | 228 | 10.069 | 62.947 | 11.389 | 1.00 | 30.81 | C |
| ATOM | 1577 | CD2 | LEU | A | 228 | 9.321 | 64.917 | 12.784 | 1.00 | 30.15 | C |
| ATOM | 1578 | C | LEU | A | 228 | 10.315 | 62.661 | 16.394 | 1.00 | 29.84 | C |
| ATOM | 1579 | O | LEU | A | 228 | 9.227 | 62.815 | 16.958 | 1.00 | 30.50 | O |
| ATOM | 1580 | N | GLY | A | 229 | 11.206 | 61.751 | 16.765 | 1.00 | 29.67 | N |
| ATOM | 1581 | CA | GLY | A | 229 | 11.008 | 60.895 | 17.920 | 1.00 | 29.67 | C |
| ATOM | 1582 | C | GLY | A | 229 | 10.994 | 61.723 | 19.208 | 1.00 | 30.52 | C |
| ATOM | 1583 | O | GLY | A | 229 | 10.169 | 61.486 | 20.105 | 1.00 | 28.98 | O |
| ATOM | 1584 | N | ILE | A | 230 | 11.920 | 62.670 | 19.307 | 1.00 | 30.54 | N |
| ATOM | 1585 | CA | ILE | A | 230 | 11.986 | 63.587 | 20.459 | 1.00 | 31.11 | C |
| ATOM | 1586 | CB | ILE | A | 230 | 13.199 | 64.564 | 20.334 | 1.00 | 31.68 | C |
| ATOM | 1587 | CG1 | ILE | A | 230 | 14.526 | 63.792 | 20.281 | 1.00 | 30.92 | C |
| ATOM | 1588 | CD1 | ILE | A | 230 | 14.824 | 62.992 | 21.546 | 1.00 | 30.66 | C |
| ATOM | 1589 | CG2 | ILE | A | 230 | 13.229 | 65.553 | 21.533 | 1.00 | 30.61 | C |
| ATOM | 1590 | C | ILE | A | 230 | 10.693 | 64.397 | 20.532 | 1.00 | 31.56 | C |
| ATOM | 1591 | O | ILE | A | 230 | 10.050 | 64.488 | 21.596 | 1.00 | 31.09 | O |
| ATOM | 1592 | N | LEU | A | 231 | 10.289 | 64.928 | 19.373 | 1.00 | 30.97 | N |
| ATOM | 1593 | CA | LEU | A | 231 | 9.050 | 65.711 | 19.257 | 1.00 | 30.14 | C |
| ATOM | 1594 | CB | LEU | A | 231 | 8.894 | 66.239 | 17.828 | 1.00 | 30.10 | C |
| ATOM | 1595 | CG | LEU | A | 231 | 7.627 | 67.043 | 17.556 | 1.00 | 32.25 | C |
| ATOM | 1596 | CD1 | LEU | A | 231 | 7.733 | 68.372 | 18.310 | 1.00 | 30.47 | C |
| ATOM | 1597 | CD2 | LEU | A | 231 | 7.419 | 67.246 | 16.065 | 1.00 | 30.92 | C |
| ATOM | 1598 | C | LEU | A | 231 | 7.798 | 64.950 | 19.689 | 1.00 | 30.59 | C |
| ATOM | 1599 | O | LEU | A | 231 | 6.949 | 65.484 | 20.439 | 1.00 | 30.81 | O |
| ATOM | 1600 | N | LEU | A | 232 | 7.655 | 63.721 | 19.210 | 1.00 | 29.58 | N |
| ATOM | 1601 | CA | LEU | A | 232 | 6.499 | 62.916 | 19.552 | 1.00 | 30.41 | C |
| ATOM | 1602 | CB | LEU | A | 232 | 6.470 | 61.609 | 18.745 | 1.00 | 30.17 | C |
| ATOM | 1603 | CG | LEU | A | 232 | 5.301 | 60.642 | 19.033 | 1.00 | 30.99 | C |
| ATOM | 1604 | CD1 | LEU | A | 232 | 3.947 | 61.346 | 18.919 | 1.00 | 33.55 | C |
| ATOM | 1605 | CD2 | LEU | A | 232 | 5.359 | 59.465 | 18.073 | 1.00 | 31.95 | C |
| ATOM | 1606 | C | LEU | A | 232 | 6.439 | 62.630 | 21.062 | 1.00 | 30.78 | C |
| ATOM | 1607 | O | LEU | A | 232 | 5.371 | 62.721 | 21.667 | 1.00 | 31.42 | O |
| ATOM | 1608 | N | TYR | A | 233 | 7.571 | 62.272 | 21.650 | 1.00 | 30.02 | N |
| ATOM | 1609 | CA | TYR | A | 233 | 7.646 | 62.042 | 23.103 | 1.00 | 30.55 | C |
| ATOM | 1610 | CB | TYR | A | 233 | 9.068 | 61.662 | 23.526 | 1.00 | 30.26 | C |
| ATOM | 1611 | CG | TYR | A | 233 | 9.209 | 61.373 | 25.008 | 1.00 | 28.93 | C |
| ATOM | 1612 | CD1 | TYR | A | 233 | 9.255 | 62.416 | 25.930 | 1.00 | 29.15 | C |
| ATOM | 1613 | CE1 | TYR | A | 233 | 9.353 | 62.171 | 27.311 | 1.00 | 28.65 | C |
| ATOM | 1614 | CZ | TYR | A | 233 | 9.407 | 60.882 | 27.769 | 1.00 | 31.81 | C |
| ATOM | 1615 | OH | TYR | A | 233 | 9.505 | 60.681 | 29.132 | 1.00 | 36.02 | O |
| ATOM | 1616 | CE2 | TYR | A | 233 | 9.365 | 59.801 | 26.878 | 1.00 | 30.59 | C |
| ATOM | 1617 | CD2 | TYR | A | 233 | 9.267 | 60.058 | 25.486 | 1.00 | 28.47 | C |
| ATOM | 1618 | C | TYR | A | 233 | 7.216 | 63.306 | 23.834 | 1.00 | 31.37 | C |
| ATOM | 1619 | O | TYR | A | 233 | 6.416 | 63.250 | 24.769 | 1.00 | 32.79 | O |
| ATOM | 1620 | N | ASP | A | 234 | 7.762 | 64.434 | 23.407 | 1.00 | 31.52 | N |
| ATOM | 1621 | CA | ASP | A | 234 | 7.411 | 65.750 | 23.934 | 1.00 | 33.52 | C |
| ATOM | 1622 | CB | ASP | A | 234 | 8.156 | 66.833 | 23.162 | 1.00 | 34.26 | C |
| ATOM | 1623 | CG | ASP | A | 234 | 7.951 | 68.224 | 23.745 | 1.00 | 37.82 | C |
| ATOM | 1624 | OD1 | ASP | A | 234 | 8.206 | 68.450 | 24.956 | 1.00 | 39.31 | O |
| ATOM | 1625 | OD2 | ASP | A | 234 | 7.531 | 69.156 | 23.030 | 1.00 | 39.97 | O |
| ATOM | 1626 | C | ASP | A | 234 | 5.923 | 66.023 | 23.923 | 1.00 | 34.29 | C |
| ATOM | 1627 | O | ASP | A | 234 | 5.368 | 66.524 | 24.931 | 1.00 | 35.28 | O |
| ATOM | 1628 | N | MET | A | 235 | 5.258 | 65.695 | 22.810 | 1.00 | 33.03 | N |
| ATOM | 1629 | CA | MET | A | 235 | 3.819 | 65.904 | 22.713 | 1.00 | 33.96 | C |
| ATOM | 1630 | CB | MET | A | 235 | 3.293 | 65.625 | 21.305 | 1.00 | 33.12 | C |
| ATOM | 1631 | CG | MET | A | 235 | 3.641 | 66.708 | 20.286 | 1.00 | 36.42 | C |
| ATOM | 1632 | SD | MET | A | 235 | 2.965 | 66.246 | 18.692 | 1.00 | 39.55 | S |
| ATOM | 1633 | CE | MET | A | 235 | 4.174 | 65.260 | 18.147 | 1.00 | 43.23 | C |
| ATOM | 1634 | C | MET | A | 235 | 3.020 | 65.078 | 23.703 | 1.00 | 33.96 | C |
| ATOM | 1635 | O | MET | A | 235 | 2.133 | 65.607 | 24.337 | 1.00 | 35.00 | O |
| ATOM | 1636 | N | VAL | A | 236 | 3.322 | 63.787 | 23.816 | 1.00 | 34.07 | N |
| ATOM | 1637 | CA | VAL | A | 236 | 2.518 | 62.893 | 24.660 | 1.00 | 35.02 | C |
| ATOM | 1638 | CB | VAL | A | 236 | 2.405 | 61.476 | 24.055 | 1.00 | 35.33 | C |
| ATOM | 1639 | CG1 | VAL | A | 236 | 1.757 | 61.562 | 22.673 | 1.00 | 34.30 | C |
| ATOM | 1640 | CG2 | VAL | A | 236 | 3.763 | 60.805 | 23.937 | 1.00 | 33.11 | C |
| ATOM | 1641 | C | VAL | A | 236 | 2.955 | 62.843 | 26.129 | 1.00 | 35.63 | C |
| ATOM | 1642 | O | VAL | A | 236 | 2.225 | 62.326 | 26.970 | 1.00 | 35.58 | O |
| ATOM | 1643 | N | CYS | A | 237 | 4.131 | 63.389 | 26.432 | 1.00 | 36.11 | N |
| ATOM | 1644 | CA | CYS | A | 237 | 4.642 | 63.383 | 27.814 | 1.00 | 36.98 | C |
| ATOM | 1645 | CB | CYS | A | 237 | 5.972 | 62.630 | 27.909 | 1.00 | 36.35 | C |
| ATOM | 1646 | SG | CYS | A | 237 | 5.796 | 60.844 | 27.757 | 1.00 | 38.34 | S |
| ATOM | 1647 | C | CYS | A | 237 | 4.790 | 64.778 | 28.416 | 1.00 | 37.40 | C |
| ATOM | 1648 | O | CYS | A | 237 | 4.983 | 64.907 | 29.628 | 1.00 | 38.17 | O |
| ATOM | 1649 | N | GLY | A | 238 | 4.722 | 65.812 | 27.576 | 1.00 | 36.49 | N |
| ATOM | 1650 | CA | GLY | A | 238 | 4.791 | 67.183 | 28.045 | 1.00 | 36.34 | C |
| ATOM | 1651 | C | GLY | A | 238 | 6.186 | 67.717 | 28.259 | 1.00 | 37.64 | C |
| ATOM | 1652 | O | GLY | A | 238 | 6.353 | 68.842 | 28.719 | 1.00 | 37.75 | O |
| ATOM | 1653 | N | ASP | A | 239 | 7.198 | 66.916 | 27.939 | 1.00 | 38.11 | N |
| ATOM | 1654 | CA | ASP | A | 239 | 8.580 | 67.369 | 28.009 | 1.00 | 39.21 | C |
| ATOM | 1655 | CB | ASP | A | 239 | 9.056 | 67.339 | 29.458 | 1.00 | 40.74 | C |
| ATOM | 1656 | CG | ASP | A | 239 | 10.214 | 68.302 | 29.735 | 1.00 | 45.40 | C |
| ATOM | 1657 | OD1 | ASP | A | 239 | 10.586 | 69.135 | 28.867 | 1.00 | 49.01 | O |
| ATOM | 1658 | OD2 | ASP | A | 239 | 10.822 | 68.274 | 30.828 | 1.00 | 50.33 | O |
| ATOM | 1659 | C | ASP | A | 239 | 9.418 | 66.434 | 27.142 | 1.00 | 39.28 | C |
| ATOM | 1660 | O | ASP | A | 239 | 8.957 | 65.354 | 26.769 | 1.00 | 39.18 | O |
| ATOM | 1661 | N | ILE | A | 240 | 10.630 | 66.856 | 26.809 | 1.00 | 39.54 | N |
| ATOM | 1662 | CA | ILE | A | 240 | 11.529 | 66.066 | 25.983 | 1.00 | 39.95 | C |
| ATOM | 1663 | CB | ILE | A | 240 | 12.641 | 66.964 | 25.440 | 1.00 | 40.83 | C |
| ATOM | 1664 | CG1 | ILE | A | 240 | 13.306 | 67.740 | 26.578 | 1.00 | 41.83 | C |
| ATOM | 1665 | CD1 | ILE | A | 240 | 14.455 | 68.627 | 26.125 | 1.00 | 44.95 | C |
| ATOM | 1666 | CG2 | ILE | A | 240 | 12.092 | 67.911 | 24.344 | 1.00 | 39.77 | C |
| ATOM | 1667 | C | ILE | A | 240 | 12.106 | 64.916 | 26.827 | 1.00 | 40.26 | C |
| ATOM | 1668 | O | ILE | A | 240 | 12.187 | 65.046 | 28.049 | 1.00 | 40.89 | O |
| ATOM | 1669 | N | PRO | A | 241 | 12.470 | 63.791 | 26.210 | 1.00 | 40.14 | N |
| ATOM | 1670 | CA | PRO | A | 241 | 12.941 | 62.620 | 26.971 | 1.00 | 41.03 | C |
| ATOM | 1671 | CB | PRO | A | 241 | 12.879 | 61.494 | 25.932 | 1.00 | 40.79 | C |
| ATOM | 1672 | CG | PRO | A | 241 | 13.150 | 62.187 | 24.622 | 1.00 | 39.44 | C |
| ATOM | 1673 | CD | PRO | A | 241 | 12.444 | 63.518 | 24.757 | 1.00 | 39.54 | C |
| ATOM | 1674 | C | PRO | A | 241 | 14.361 | 62.737 | 27.548 | 1.00 | 42.89 | C |
| ATOM | 1675 | O | PRO | A | 241 | 14.639 | 62.109 | 28.571 | 1.00 | 42.98 | O |
| ATOM | 1676 | N | PHE | A | 242 | 15.243 | 63.508 | 26.912 | 1.00 | 44.80 | N |
| ATOM | 1677 | CA | PHE | A | 242 | 16.644 | 63.555 | 27.340 | 1.00 | 46.51 | C |
| ATOM | 1678 | CB | PHE | A | 242 | 17.589 | 62.944 | 26.285 | 1.00 | 45.41 | C |
| ATOM | 1679 | CG | PHE | A | 242 | 17.145 | 61.617 | 25.735 | 1.00 | 43.12 | C |
| ATOM | 1680 | CD1 | PHE | A | 242 | 16.885 | 60.545 | 26.578 | 1.00 | 42.42 | C |
| ATOM | 1681 | CE1 | PHE | A | 242 | 16.496 | 59.313 | 26.068 | 1.00 | 41.08 | C |
| ATOM | 1682 | CZ | PHE | A | 242 | 16.367 | 59.148 | 24.676 | 1.00 | 43.10 | C |
| ATOM | 1683 | CE2 | PHE | A | 242 | 16.618 | 60.222 | 23.824 | 1.00 | 40.87 | C |
| ATOM | 1684 | CD2 | PHE | A | 242 | 17.012 | 61.438 | 24.350 | 1.00 | 42.39 | C |
| ATOM | 1685 | C | PHE | A | 242 | 17.104 | 64.973 | 27.639 | 1.00 | 48.94 | C |
| ATOM | 1686 | O | PHE | A | 242 | 16.783 | 65.913 | 26.903 | 1.00 | 48.57 | O |
| ATOM | 1687 | N | GLU | A | 243 | 17.884 | 65.112 | 28.714 | 1.00 | 52.57 | N |
| ATOM | 1688 | CA | GLU | A | 243 | 18.514 | 66.391 | 29.046 | 1.00 | 55.94 | C |
| ATOM | 1689 | CB | GLU | A | 243 | 18.204 | 66.793 | 30.496 | 1.00 | 57.25 | C |
| ATOM | 1690 | CG | GLU | A | 243 | 16.930 | 67.634 | 30.664 | 1.00 | 62.51 | C |
| ATOM | 1691 | CD | GLU | A | 243 | 16.911 | 68.912 | 29.814 | 1.00 | 68.14 | C |
| ATOM | 1692 | OE1 | GLU | A | 243 | 17.901 | 69.697 | 29.854 | 1.00 | 69.83 | O |
| ATOM | 1693 | OE2 | GLU | A | 243 | 15.894 | 69.140 | 29.104 | 1.00 | 69.55 | O |
| ATOM | 1694 | C | GLU | A | 243 | 20.022 | 66.364 | 28.813 | 1.00 | 56.70 | C |
| ATOM | 1695 | O | GLU | A | 243 | 20.596 | 67.329 | 28.291 | 1.00 | 57.61 | O |
| ATOM | 1696 | N | HIS | A | 244 | 20.654 | 65.250 | 29.169 | 1.00 | 57.00 | N |
| ATOM | 1697 | CA | HIS | A | 244 | 22.111 | 65.145 | 29.128 | 1.00 | 57.57 | C |
| ATOM | 1698 | CB | HIS | A | 244 | 22.634 | 64.653 | 30.484 | 1.00 | 57.93 | C |
| ATOM | 1699 | CG | HIS | A | 244 | 22.177 | 65.491 | 31.641 | 1.00 | 60.15 | C |
| ATOM | 1700 | ND1 | HIS | A | 244 | 21.243 | 65.040 | 32.563 | 1.00 | 61.46 | N |
| ATOM | 1701 | CE1 | HIS | A | 244 | 21.021 | 65.986 | 33.459 | 1.00 | 61.53 | C |
| ATOM | 1702 | NE2 | HIS | A | 244 | 21.772 | 67.040 | 33.145 | 1.00 | 61.93 | N |
| ATOM | 1703 | CD2 | HIS | A | 244 | 22.501 | 66.761 | 32.008 | 1.00 | 60.89 | C |
| ATOM | 1704 | C | HIS | A | 244 | 22.632 | 64.251 | 27.999 | 1.00 | 57.12 | C |
| ATOM | 1705 | O | HIS | A | 244 | 21.946 | 63.321 | 27.564 | 1.00 | 56.42 | O |
| ATOM | 1706 | N | ASP | A | 245 | 23.850 | 64.550 | 27.542 | 1.00 | 56.65 | N |
| ATOM | 1707 | CA | ASP | A | 245 | 24.536 | 63.778 | 26.508 | 1.00 | 56.51 | C |
| ATOM | 1708 | CB | ASP | A | 245 | 25.982 | 64.254 | 26.364 | 1.00 | 56.84 | C |
| ATOM | 1709 | CG | ASP | A | 245 | 26.093 | 65.551 | 25.602 | 1.00 | 58.28 | C |
| ATOM | 1710 | OD1 | ASP | A | 245 | 25.109 | 66.322 | 25.555 | 1.00 | 60.57 | O |
| ATOM | 1711 | OD2 | ASP | A | 245 | 27.132 | 65.889 | 25.003 | 1.00 | 61.68 | O |
| ATOM | 1712 | C | ASP | A | 245 | 24.520 | 62.289 | 26.792 | 1.00 | 55.85 | C |
| ATOM | 1713 | O | ASP | A | 245 | 24.240 | 61.487 | 25.902 | 1.00 | 55.84 | O |
| ATOM | 1714 | N | GLU | A | 246 | 24.807 | 61.926 | 28.038 | 1.00 | 55.12 | N |
| ATOM | 1715 | CA | GLU | A | 246 | 24.814 | 60.528 | 28.473 | 1.00 | 54.57 | C |
| ATOM | 1716 | CB | GLU | A | 246 | 25.227 | 60.414 | 29.948 | 1.00 | 55.49 | C |
| ATOM | 1717 | CG | GLU | A | 246 | 26.247 | 61.439 | 30.419 | 1.00 | 59.92 | C |
| ATOM | 1718 | CD | GLU | A | 246 | 25.601 | 62.745 | 30.853 | 1.00 | 64.57 | C |
| ATOM | 1719 | OE1 | GLU | A | 246 | 24.864 | 62.732 | 31.873 | 1.00 | 66.43 | O |
| ATOM | 1720 | OE2 | GLU | A | 246 | 25.824 | 63.779 | 30.165 | 1.00 | 66.00 | O |
| ATOM | 1721 | C | GLU | A | 246 | 23.464 | 59.838 | 28.284 | 1.00 | 52.76 | C |
| ATOM | 1722 | O | GLU | A | 246 | 23.405 | 58.637 | 27.998 | 1.00 | 51.94 | O |
| ATOM | 1723 | N | GLU | A | 247 | 22.381 | 60.583 | 28.491 | 1.00 | 51.08 | N |
| ATOM | 1724 | CA | GLU | A | 247 | 21.037 | 60.031 | 28.289 | 1.00 | 50.00 | C |
| ATOM | 1725 | CB | GLU | A | 247 | 19.982 | 60.949 | 28.888 | 1.00 | 50.91 | C |
| ATOM | 1726 | CG | GLU | A | 247 | 20.048 | 61.069 | 30.398 | 1.00 | 54.76 | C |
| ATOM | 1727 | CD | GLU | A | 247 | 19.070 | 62.089 | 30.919 | 1.00 | 59.14 | C |
| ATOM | 1728 | OE1 | GLU | A | 247 | 19.189 | 63.281 | 30.568 | 1.00 | 61.88 | O |
| ATOM | 1729 | OE2 | GLU | A | 247 | 18.172 | 61.693 | 31.672 | 1.00 | 63.68 | O |
| ATOM | 1730 | C | GLU | A | 247 | 20.734 | 59.785 | 26.810 | 1.00 | 47.56 | C |
| ATOM | 1731 | O | GLU | A | 247 | 20.177 | 58.757 | 26.463 | 1.00 | 46.72 | O |
| ATOM | 1732 | N | ILE | A | 248 | 21.102 | 60.738 | 25.957 | 1.00 | 46.28 | N |
| ATOM | 1733 | CA | ILE | A | 248 | 20.964 | 60.598 | 24.498 | 1.00 | 46.12 | C |
| ATOM | 1734 | CB | ILE | A | 248 | 21.446 | 61.876 | 23.754 | 1.00 | 45.90 | C |
| ATOM | 1735 | CG1 | ILE | A | 248 | 20.599 | 63.092 | 24.141 | 1.00 | 44.91 | C |
| ATOM | 1736 | CD1 | ILE | A | 248 | 21.110 | 64.419 | 23.588 | 1.00 | 44.29 | C |
| ATOM | 1737 | CG2 | ILE | A | 248 | 21.444 | 61.658 | 22.233 | 1.00 | 45.48 | C |
| ATOM | 1738 | C | ILE | A | 248 | 21.741 | 59.390 | 23.988 | 1.00 | 46.47 | C |
| ATOM | 1739 | O | ILE | A | 248 | 21.221 | 58.613 | 23.199 | 1.00 | 46.50 | O |
| ATOM | 1740 | N | ILE | A | 249 | 22.977 | 59.223 | 24.462 | 1.00 | 46.70 | N |
| ATOM | 1741 | CA | ILE | A | 249 | 23.845 | 58.119 | 24.021 | 1.00 | 47.73 | C |
| ATOM | 1742 | CB | ILE | A | 249 | 25.315 | 58.342 | 24.516 | 1.00 | 48.27 | C |
| ATOM | 1743 | CG1 | ILE | A | 249 | 25.882 | 59.634 | 23.929 | 1.00 | 50.01 | C |
| ATOM | 1744 | CD1 | ILE | A | 249 | 27.162 | 60.114 | 24.638 | 1.00 | 54.07 | C |
| ATOM | 1745 | CG2 | ILE | A | 249 | 26.208 | 57.167 | 24.127 | 1.00 | 49.80 | C |
| ATOM | 1746 | C | ILE | A | 249 | 23.344 | 56.752 | 24.463 | 1.00 | 47.21 | C |
| ATOM | 1747 | O | ILE | A | 249 | 23.473 | 55.754 | 23.735 | 1.00 | 47.14 | O |
| ATOM | 1748 | N | ARG | A | 250 | 22.798 | 56.697 | 25.671 | 1.00 | 46.59 | N |
| ATOM | 1749 | CA | ARG | A | 250 | 22.259 | 55.454 | 26.197 | 1.00 | 46.70 | C |
| ATOM | 1750 | CB | ARG | A | 250 | 22.052 | 55.573 | 27.712 | 1.00 | 46.73 | C |
| ATOM | 1751 | CG | ARG | A | 250 | 21.612 | 54.297 | 28.415 | 1.00 | 47.47 | C |
| ATOM | 1752 | CD | ARG | A | 250 | 21.702 | 54.415 | 29.942 | 1.00 | 49.11 | C |
| ATOM | 1753 | NE | ARG | A | 250 | 21.290 | 53.191 | 30.631 | 1.00 | 50.87 | N |
| ATOM | 1754 | CZ | ARG | A | 250 | 20.217 | 53.076 | 31.429 | 1.00 | 50.66 | C |
| ATOM | 1755 | NH1 | ARG | A | 250 | 19.412 | 54.117 | 31.656 | 1.00 | 46.65 | N |
| ATOM | 1756 | NH2 | ARG | A | 250 | 19.955 | 51.909 | 32.006 | 1.00 | 50.26 | N |
| ATOM | 1757 | C | ARG | A | 250 | 20.949 | 55.097 | 25.483 | 1.00 | 46.42 | C |
| ATOM | 1758 | O | ARG | A | 250 | 20.617 | 53.922 | 25.352 | 1.00 | 47.00 | O |
| ATOM | 1759 | N | GLY | A | 251 | 20.224 | 56.113 | 25.018 | 1.00 | 46.84 | N |
| ATOM | 1760 | CA | GLY | A | 251 | 18.982 | 55.936 | 24.269 | 1.00 | 47.26 | C |
| ATOM | 1761 | C | GLY | A | 251 | 17.855 | 55.180 | 24.968 | 1.00 | 47.36 | C |
| ATOM | 1762 | O | GLY | A | 251 | 16.936 | 54.702 | 24.318 | 1.00 | 47.71 | O |
| ATOM | 1763 | N | GLN | A | 252 | 17.921 | 55.067 | 26.290 | 1.00 | 46.77 | N |
| ATOM | 1764 | CA | GLN | A | 252 | 16.872 | 54.400 | 27.058 | 1.00 | 46.61 | C |
| ATOM | 1765 | CB | GLN | A | 252 | 17.438 | 53.965 | 28.410 | 1.00 | 47.77 | C |
| ATOM | 1766 | CG | GLN | A | 252 | 16.745 | 52.797 | 29.034 | 1.00 | 53.27 | C |
| ATOM | 1767 | CD | GLN | A | 252 | 17.362 | 51.495 | 28.593 | 1.00 | 58.82 | C |
| ATOM | 1768 | OE1 | GLN | A | 252 | 16.922 | 50.902 | 27.587 | 1.00 | 62.58 | O |
| ATOM | 1769 | NE2 | GLN | A | 252 | 18.381 | 51.040 | 29.328 | 1.00 | 59.71 | N |
| ATOM | 1770 | C | GLN | A | 252 | 15.720 | 55.388 | 27.264 | 1.00 | 44.36 | C |
| ATOM | 1771 | O | GLN | A | 252 | 15.914 | 56.458 | 27.842 | 1.00 | 43.90 | O |
| ATOM | 1772 | N | VAL | A | 253 | 14.534 | 55.036 | 26.789 | 1.00 | 42.26 | N |
| ATOM | 1773 | CA | VAL | A | 253 | 13.366 | 55.917 | 26.887 | 1.00 | 41.30 | C |
| ATOM | 1774 | CB | VAL | A | 253 | 12.515 | 55.900 | 25.574 | 1.00 | 40.85 | C |
| ATOM | 1775 | CG1 | VAL | A | 253 | 11.398 | 56.917 | 25.657 | 1.00 | 41.13 | C |
| ATOM | 1776 | CG2 | VAL | A | 253 | 13.386 | 56.184 | 24.330 | 1.00 | 41.05 | C |
| ATOM | 1777 | C | VAL | A | 253 | 12.452 | 55.558 | 28.080 | 1.00 | 40.21 | C |
| ATOM | 1778 | O | VAL | A | 253 | 11.870 | 54.475 | 28.128 | 1.00 | 39.18 | O |
| ATOM | 1779 | N | PHE | A | 254 | 12.312 | 56.494 | 29.004 | 1.00 | 40.53 | N |
| ATOM | 1780 | CA | PHE | A | 254 | 11.415 | 56.340 | 30.147 | 1.00 | 41.30 | C |
| ATOM | 1781 | CB | PHE | A | 254 | 12.125 | 56.749 | 31.446 | 1.00 | 42.46 | C |
| ATOM | 1782 | CG | PHE | A | 254 | 11.181 | 56.979 | 32.597 | 1.00 | 45.89 | C |
| ATOM | 1783 | CD1 | PHE | A | 254 | 10.698 | 55.890 | 33.354 | 1.00 | 48.15 | C |
| ATOM | 1784 | CE1 | PHE | A | 254 | 9.794 | 56.087 | 34.453 | 1.00 | 46.65 | C |
| ATOM | 1785 | CZ | PHE | A | 254 | 9.368 | 57.391 | 34.762 | 1.00 | 47.61 | C |
| ATOM | 1786 | CE2 | PHE | A | 254 | 9.840 | 58.499 | 33.990 | 1.00 | 48.28 | C |
| ATOM | 1787 | CD2 | PHE | A | 254 | 10.742 | 58.287 | 32.922 | 1.00 | 47.87 | C |
| ATOM | 1788 | C | PHE | A | 254 | 10.192 | 57.216 | 29.960 | 1.00 | 40.77 | C |
| ATOM | 1789 | O | PHE | A | 254 | 10.324 | 58.388 | 29.630 | 1.00 | 40.62 | O |
| ATOM | 1790 | N | PHE | A | 255 | 9.011 | 56.656 | 30.202 | 1.00 | 39.76 | N |
| ATOM | 1791 | CA | PHE | A | 255 | 7.772 | 57.377 | 30.041 | 1.00 | 40.05 | C |
| ATOM | 1792 | CB | PHE | A | 255 | 6.744 | 56.512 | 29.293 | 1.00 | 38.87 | C |
| ATOM | 1793 | CG | PHE | A | 255 | 7.047 | 56.408 | 27.844 | 1.00 | 38.12 | C |
| ATOM | 1794 | CD1 | PHE | A | 255 | 6.520 | 57.332 | 26.945 | 1.00 | 37.51 | C |
| ATOM | 1795 | CE1 | PHE | A | 255 | 6.834 | 57.267 | 25.588 | 1.00 | 37.08 | C |
| ATOM | 1796 | CZ | PHE | A | 255 | 7.715 | 56.277 | 25.126 | 1.00 | 38.31 | C |
| ATOM | 1797 | CE2 | PHE | A | 255 | 8.251 | 55.353 | 26.034 | 1.00 | 37.42 | C |
| ATOM | 1798 | CD2 | PHE | A | 255 | 7.917 | 55.429 | 27.379 | 1.00 | 36.49 | C |
| ATOM | 1799 | C | PHE | A | 255 | 7.233 | 57.901 | 31.355 | 1.00 | 40.75 | C |
| ATOM | 1800 | O | PHE | A | 255 | 6.974 | 57.139 | 32.280 | 1.00 | 41.63 | O |
| ATOM | 1801 | N | ARG | A | 256 | 7.078 | 59.214 | 31.414 | 1.00 | 42.10 | N |
| ATOM | 1802 | CA | ARG | A | 256 | 6.613 | 59.911 | 32.614 | 1.00 | 43.68 | C |
| ATOM | 1803 | CB | ARG | A | 256 | 7.284 | 61.291 | 32.722 | 1.00 | 44.33 | C |
| ATOM | 1804 | CG | ARG | A | 256 | 7.050 | 62.233 | 31.549 | 1.00 | 46.48 | C |
| ATOM | 1805 | CD | ARG | A | 256 | 7.915 | 63.508 | 31.606 | 1.00 | 49.15 | C |
| ATOM | 1806 | NE | ARG | A | 256 | 9.248 | 63.277 | 31.034 | 1.00 | 53.26 | N |
| ATOM | 1807 | CZ | ARG | A | 256 | 10.334 | 64.018 | 31.293 | 1.00 | 54.62 | C |
| ATOM | 1808 | NH1 | ARG | A | 256 | 10.260 | 65.060 | 32.133 | 1.00 | 55.57 | N |
| ATOM | 1809 | NH2 | ARG | A | 256 | 11.502 | 63.720 | 30.716 | 1.00 | 52.61 | N |
| ATOM | 1810 | C | ARG | A | 256 | 5.096 | 60.051 | 32.658 | 1.00 | 43.59 | C |
| ATOM | 1811 | O | ARG | A | 256 | 4.525 | 60.251 | 33.724 | 1.00 | 44.72 | O |
| ATOM | 1812 | N | GLN | A | 257 | 4.454 | 59.930 | 31.498 | 1.00 | 42.63 | N |
| ATOM | 1813 | CA | GLN | A | 257 | 3.001 | 59.949 | 31.403 | 1.00 | 41.06 | C |
| ATOM | 1814 | CB | GLN | A | 257 | 2.538 | 61.061 | 30.445 | 1.00 | 42.46 | C |
| ATOM | 1815 | CG | GLN | A | 257 | 2.890 | 62.451 | 30.901 | 1.00 | 46.35 | C |
| ATOM | 1816 | CD | GLN | A | 257 | 1.908 | 62.984 | 31.916 | 1.00 | 51.18 | C |
| ATOM | 1817 | OE1 | GLN | A | 257 | 0.693 | 62.917 | 31.711 | 1.00 | 52.75 | O |
| ATOM | 1818 | NE2 | GLN | A | 257 | 2.428 | 63.504 | 33.020 | 1.00 | 55.05 | N |
| ATOM | 1819 | C | GLN | A | 257 | 2.510 | 58.618 | 30.872 | 1.00 | 38.68 | C |
| ATOM | 1820 | O | GLN | A | 257 | 3.267 | 57.849 | 30.300 | 1.00 | 38.45 | O |
| ATOM | 1821 | N | ARG | A | 258 | 1.226 | 58.353 | 31.047 | 1.00 | 36.14 | N |
| ATOM | 1822 | CA | ARG | A | 258 | 0.614 | 57.177 | 30.479 | 1.00 | 36.06 | C |
| ATOM | 1823 | CB | ARG | A | 258 | −0.820 | 57.048 | 30.997 | 1.00 | 34.77 | C |
| ATOM | 1824 | CG | ARG | A | 258 | −1.402 | 55.659 | 30.847 | 1.00 | 39.04 | C |
| ATOM | 1825 | CD | ARG | A | 258 | −1.624 | 55.230 | 29.442 | 1.00 | 40.99 | C |
| ATOM | 1826 | NE | ARG | A | 258 | −1.799 | 53.789 | 29.300 | 1.00 | 40.39 | N |
| ATOM | 1827 | CZ | ARG | A | 258 | −2.327 | 53.219 | 28.215 | 1.00 | 43.89 | C |
| ATOM | 1828 | NH1 | ARG | A | 258 | −2.730 | 53.966 | 27.158 | 1.00 | 45.06 | N |
| ATOM | 1829 | NH2 | ARG | A | 258 | −2.444 | 51.899 | 28.162 | 1.00 | 40.81 | N |
| ATOM | 1830 | C | ARG | A | 258 | 0.599 | 57.345 | 28.950 | 1.00 | 35.62 | C |
| ATOM | 1831 | O | ARG | A | 258 | 0.071 | 58.325 | 28.463 | 1.00 | 35.32 | O |
| ATOM | 1832 | N | VAL | A | 259 | 1.159 | 56.385 | 28.221 | 1.00 | 34.94 | N |
| ATOM | 1833 | CA | VAL | A | 259 | 1.223 | 56.440 | 26.755 | 1.00 | 34.87 | C |
| ATOM | 1834 | CB | VAL | A | 259 | 2.629 | 56.926 | 26.277 | 1.00 | 35.54 | C |
| ATOM | 1835 | CG1 | VAL | A | 259 | 2.782 | 56.824 | 24.747 | 1.00 | 34.21 | C |
| ATOM | 1836 | CG2 | VAL | A | 259 | 2.902 | 58.365 | 26.752 | 1.00 | 33.65 | C |
| ATOM | 1837 | C | VAL | A | 259 | 0.967 | 55.033 | 26.235 | 1.00 | 35.77 | C |
| ATOM | 1838 | O | VAL | A | 259 | 1.579 | 54.062 | 26.728 | 1.00 | 35.74 | O |
| ATOM | 1839 | N | SER | A | 260 | 0.055 | 54.901 | 25.267 | 1.00 | 35.24 | N |
| ATOM | 1840 | CA | SER | A | 260 | −0.269 | 53.601 | 24.698 | 1.00 | 36.49 | C |
| ATOM | 1841 | CB | SER | A | 260 | −1.247 | 53.749 | 23.525 | 1.00 | 36.47 | C |
| ATOM | 1842 | OG | SER | A | 260 | −0.608 | 54.285 | 22.377 | 1.00 | 37.04 | O |
| ATOM | 1843 | C | SER | A | 260 | 0.973 | 52.855 | 24.226 | 1.00 | 37.15 | C |
| ATOM | 1844 | O | SER | A | 260 | 1.981 | 53.465 | 23.874 | 1.00 | 37.30 | O |
| ATOM | 1845 | N | SER | A | 261 | 0.876 | 51.533 | 24.178 | 1.00 | 37.42 | N |
| ATOM | 1846 | CA | SER | A | 261 | 2.000 | 50.701 | 23.767 | 1.00 | 37.73 | C |
| ATOM | 1847 | CB | SER | A | 261 | 1.659 | 49.225 | 23.941 | 1.00 | 38.27 | C |
| ATOM | 1848 | OG | SER | A | 261 | 1.475 | 48.939 | 25.316 | 1.00 | 42.42 | O |
| ATOM | 1849 | C | SER | A | 261 | 2.399 | 50.965 | 22.325 | 1.00 | 37.68 | C |
| ATOM | 1850 | O | SER | A | 261 | 3.578 | 50.914 | 21.997 | 1.00 | 36.60 | O |
| ATOM | 1851 | N | GLU | A | 262 | 1.413 | 51.260 | 21.478 | 1.00 | 37.69 | N |
| ATOM | 1852 | CA | GLU | A | 262 | 1.662 | 51.578 | 20.080 | 1.00 | 38.33 | C |
| ATOM | 1853 | CB | GLU | A | 262 | 0.343 | 51.655 | 19.307 | 1.00 | 40.07 | C |
| ATOM | 1854 | CG | GLU | A | 262 | −0.522 | 50.401 | 19.444 | 1.00 | 47.46 | C |
| ATOM | 1855 | CD | GLU | A | 262 | −1.138 | 49.954 | 18.125 | 1.00 | 55.14 | C |
| ATOM | 1856 | OE1 | GLU | A | 262 | −1.716 | 50.811 | 17.407 | 1.00 | 58.98 | O |
| ATOM | 1857 | OE2 | GLU | A | 262 | −1.058 | 48.740 | 17.799 | 1.00 | 59.70 | O |
| ATOM | 1858 | C | GLU | A | 262 | 2.469 | 52.878 | 19.945 | 1.00 | 36.65 | C |
| ATOM | 1859 | O | GLU | A | 262 | 3.442 | 52.911 | 19.227 | 1.00 | 36.27 | O |
| ATOM | 1860 | N | CYS | A | 263 | 2.073 | 53.931 | 20.651 | 1.00 | 35.34 | N |
| ATOM | 1861 | CA | CYS | A | 263 | 2.822 | 55.188 | 20.621 | 1.00 | 34.46 | C |
| ATOM | 1862 | CB | CYS | A | 263 | 2.051 | 56.272 | 21.363 | 1.00 | 34.30 | C |
| ATOM | 1863 | SG | CYS | A | 263 | 2.728 | 57.931 | 21.207 | 1.00 | 34.38 | S |
| ATOM | 1864 | C | CYS | A | 263 | 4.250 | 55.021 | 21.181 | 1.00 | 34.46 | C |
| ATOM | 1865 | O | CYS | A | 263 | 5.221 | 55.477 | 20.556 | 1.00 | 32.45 | O |
| ATOM | 1866 | N | GLN | A | 264 | 4.385 | 54.325 | 22.321 | 1.00 | 33.42 | N |
| ATOM | 1867 | CA | GLN | A | 264 | 5.715 | 54.008 | 22.859 | 1.00 | 33.11 | C |
| ATOM | 1868 | CB | GLN | A | 264 | 5.629 | 53.110 | 24.097 | 1.00 | 33.38 | C |
| ATOM | 1869 | CG | GLN | A | 264 | 5.022 | 53.781 | 25.364 | 1.00 | 35.44 | C |
| ATOM | 1870 | CD | GLN | A | 264 | 5.296 | 52.968 | 26.647 | 1.00 | 37.59 | C |
| ATOM | 1871 | OE1 | GLN | A | 264 | 6.162 | 52.098 | 26.655 | 1.00 | 39.41 | O |
| ATOM | 1872 | NE2 | GLN | A | 264 | 4.566 | 53.262 | 27.717 | 1.00 | 33.63 | N |
| ATOM | 1873 | C | GLN | A | 264 | 6.578 | 53.314 | 21.795 | 1.00 | 33.15 | C |
| ATOM | 1874 | O | GLN | A | 264 | 7.753 | 53.689 | 21.606 | 1.00 | 32.43 | O |
| ATOM | 1875 | N | HIS | A | 265 | 6.001 | 52.322 | 21.110 | 1.00 | 32.53 | N |
| ATOM | 1876 | CA | HIS | A | 265 | 6.710 | 51.575 | 20.068 | 1.00 | 34.99 | C |
| ATOM | 1877 | CB | HIS | A | 265 | 5.836 | 50.455 | 19.469 | 1.00 | 36.23 | C |
| ATOM | 1878 | CG | HIS | A | 265 | 6.515 | 49.687 | 18.369 | 1.00 | 39.71 | C |
| ATOM | 1879 | ND1 | HIS | A | 265 | 6.481 | 50.086 | 17.050 | 1.00 | 40.80 | N |
| ATOM | 1880 | CE1 | HIS | A | 265 | 7.189 | 49.244 | 16.314 | 1.00 | 42.05 | C |
| ATOM | 1881 | NE2 | HIS | A | 265 | 7.687 | 48.312 | 17.110 | 1.00 | 42.46 | N |
| ATOM | 1882 | CD2 | HIS | A | 265 | 7.286 | 48.570 | 18.402 | 1.00 | 42.81 | C |
| ATOM | 1883 | C | HIS | A | 265 | 7.226 | 52.508 | 18.968 | 1.00 | 34.14 | C |
| ATOM | 1884 | O | HIS | A | 265 | 8.410 | 52.463 | 18.613 | 1.00 | 34.55 | O |
| ATOM | 1885 | N | LEU | A | 266 | 6.355 | 53.372 | 18.445 | 1.00 | 32.99 | N |
| ATOM | 1886 | CA | LEU | A | 266 | 6.778 | 54.306 | 17.394 | 1.00 | 32.41 | C |
| ATOM | 1887 | CB | LEU | A | 266 | 5.587 | 55.147 | 16.896 | 1.00 | 32.45 | C |
| ATOM | 1888 | CG | LEU | A | 266 | 5.863 | 56.209 | 15.818 | 1.00 | 33.18 | C |
| ATOM | 1889 | CD1 | LEU | A | 266 | 6.584 | 55.619 | 14.605 | 1.00 | 30.32 | C |
| ATOM | 1890 | CD2 | LEU | A | 266 | 4.511 | 56.820 | 15.367 | 1.00 | 30.03 | C |
| ATOM | 1891 | C | LEU | A | 266 | 7.885 | 55.211 | 17.904 | 1.00 | 31.79 | C |
| ATOM | 1892 | O | LEU | A | 266 | 8.907 | 55.417 | 17.231 | 1.00 | 31.14 | O |
| ATOM | 1893 | N | ILE | A | 267 | 7.706 | 55.750 | 19.112 | 1.00 | 31.36 | N |
| ATOM | 1894 | CA | ILE | A | 267 | 8.702 | 56.661 | 19.666 | 1.00 | 30.41 | C |
| ATOM | 1895 | CB | ILE | A | 267 | 8.273 | 57.184 | 21.052 | 1.00 | 29.95 | C |
| ATOM | 1896 | CG1 | ILE | A | 267 | 7.134 | 58.210 | 20.924 | 1.00 | 30.35 | C |
| ATOM | 1897 | CD1 | ILE | A | 267 | 6.410 | 58.513 | 22.271 | 1.00 | 30.22 | C |
| ATOM | 1898 | CG2 | ILE | A | 267 | 9.472 | 57.849 | 21.751 | 1.00 | 28.77 | C |
| ATOM | 1899 | C | ILE | A | 267 | 10.052 | 55.956 | 19.782 | 1.00 | 31.85 | C |
| ATOM | 1900 | O | ILE | A | 267 | 11.093 | 56.485 | 19.340 | 1.00 | 32.22 | O |
| ATOM | 1901 | N | ARG | A | 268 | 10.034 | 54.774 | 20.388 | 1.00 | 32.17 | N |
| ATOM | 1902 | CA | ARG | A | 268 | 11.248 | 53.988 | 20.594 | 1.00 | 34.65 | C |
| ATOM | 1903 | CB | ARG | A | 268 | 10.927 | 52.713 | 21.369 | 1.00 | 35.11 | C |
| ATOM | 1904 | CG | ARG | A | 268 | 10.707 | 52.931 | 22.864 | 1.00 | 39.57 | C |
| ATOM | 1905 | CD | ARG | A | 268 | 10.398 | 51.637 | 23.600 | 1.00 | 45.10 | C |
| ATOM | 1906 | NE | ARG | A | 268 | 9.725 | 51.866 | 24.890 | 1.00 | 48.08 | N |
| ATOM | 1907 | CZ | ARG | A | 268 | 10.370 | 52.338 | 25.935 | 1.00 | 49.97 | C |
| ATOM | 1908 | NH1 | ARG | A | 268 | 11.663 | 52.609 | 25.806 | 1.00 | 53.48 | N |
| ATOM | 1909 | NH2 | ARG | A | 268 | 9.753 | 52.551 | 27.093 | 1.00 | 48.54 | N |
| ATOM | 1910 | C | ARG | A | 268 | 11.921 | 53.622 | 19.278 | 1.00 | 34.22 | C |
| ATOM | 1911 | O | ARG | A | 268 | 13.140 | 53.491 | 19.223 | 1.00 | 34.73 | O |
| ATOM | 1912 | N | TRP | A | 269 | 11.124 | 53.464 | 18.225 | 1.00 | 34.43 | N |
| ATOM | 1913 | CA | TRP | A | 269 | 11.649 | 53.135 | 16.889 | 1.00 | 34.10 | C |
| ATOM | 1914 | CB | TRP | A | 269 | 10.503 | 52.657 | 15.992 | 1.00 | 34.69 | C |
| ATOM | 1915 | CG | TRP | A | 269 | 10.921 | 52.008 | 14.716 | 1.00 | 36.80 | C |
| ATOM | 1916 | CD1 | TRP | A | 269 | 12.191 | 51.632 | 14.352 | 1.00 | 39.30 | C |
| ATOM | 1917 | NE1 | TRP | A | 269 | 12.182 | 51.081 | 13.090 | 1.00 | 38.76 | N |
| ATOM | 1918 | CE2 | TRP | A | 269 | 10.895 | 51.071 | 12.618 | 1.00 | 37.50 | C |
| ATOM | 1919 | CD2 | TRP | A | 269 | 10.074 | 51.669 | 13.609 | 1.00 | 36.90 | C |
| ATOM | 1920 | CE3 | TRP | A | 269 | 8.703 | 51.787 | 13.359 | 1.00 | 35.95 | C |
| ATOM | 1921 | CZ3 | TRP | A | 269 | 8.197 | 51.332 | 12.136 | 1.00 | 37.55 | C |
| ATOM | 1922 | CH2 | TRP | A | 269 | 9.047 | 50.765 | 11.172 | 1.00 | 36.64 | C |
| ATOM | 1923 | CZ2 | TRP | A | 269 | 10.392 | 50.618 | 11.401 | 1.00 | 36.32 | C |
| ATOM | 1924 | C | TRP | A | 269 | 12.346 | 54.351 | 16.279 | 1.00 | 34.12 | C |
| ATOM | 1925 | O | TRP | A | 269 | 13.461 | 54.248 | 15.766 | 1.00 | 34.65 | O |
| ATOM | 1926 | N | CYS | A | 270 | 11.704 | 55.514 | 16.347 | 1.00 | 33.89 | N |
| ATOM | 1927 | CA | CYS | A | 270 | 12.315 | 56.762 | 15.881 | 1.00 | 33.18 | C |
| ATOM | 1928 | CB | CYS | A | 270 | 11.364 | 57.958 | 16.030 | 1.00 | 32.73 | C |
| ATOM | 1929 | SG | CYS | A | 270 | 9.894 | 57.933 | 14.980 | 1.00 | 34.78 | S |
| ATOM | 1930 | C | CYS | A | 270 | 13.593 | 57.085 | 16.627 | 1.00 | 33.31 | C |
| ATOM | 1931 | O | CYS | A | 270 | 14.471 | 57.759 | 16.085 | 1.00 | 33.37 | O |
| ATOM | 1932 | N | LEU | A | 271 | 13.686 | 56.635 | 17.879 | 1.00 | 32.81 | N |
| ATOM | 1933 | CA | LEU | A | 271 | 14.835 | 56.934 | 18.711 | 1.00 | 33.61 | C |
| ATOM | 1934 | CB | LEU | A | 271 | 14.405 | 57.342 | 20.143 | 1.00 | 33.79 | C |
| ATOM | 1935 | CG | LEU | A | 271 | 13.573 | 58.649 | 20.223 | 1.00 | 33.39 | C |
| ATOM | 1936 | CD1 | LEU | A | 271 | 13.178 | 58.971 | 21.668 | 1.00 | 32.58 | C |
| ATOM | 1937 | CD2 | LEU | A | 271 | 14.330 | 59.820 | 19.602 | 1.00 | 29.30 | C |
| ATOM | 1938 | C | LEU | A | 271 | 15.805 | 55.766 | 18.761 | 1.00 | 34.83 | C |
| ATOM | 1939 | O | LEU | A | 271 | 16.536 | 55.613 | 19.727 | 1.00 | 34.16 | O |
| ATOM | 1940 | N | ALA | A | 272 | 15.836 | 54.958 | 17.705 | 1.00 | 35.68 | N |
| ATOM | 1941 | CA | ALA | A | 272 | 16.796 | 53.853 | 17.658 | 1.00 | 37.00 | C |
| ATOM | 1942 | CB | ALA | A | 272 | 16.563 | 52.994 | 16.429 | 1.00 | 37.89 | C |
| ATOM | 1943 | C | ALA | A | 272 | 18.191 | 54.460 | 17.658 | 1.00 | 37.13 | C |
| ATOM | 1944 | O | ALA | A | 272 | 18.436 | 55.466 | 16.996 | 1.00 | 36.83 | O |
| ATOM | 1945 | N | LEU | A | 273 | 19.087 | 53.886 | 18.447 | 1.00 | 38.00 | N |
| ATOM | 1946 | CA | LEU | A | 273 | 20.464 | 54.378 | 18.537 | 1.00 | 39.46 | C |
| ATOM | 1947 | CB | LEU | A | 273 | 21.266 | 53.531 | 19.532 | 1.00 | 39.79 | C |
| ATOM | 1948 | CG | LEU | A | 273 | 20.990 | 53.795 | 21.011 | 1.00 | 40.61 | C |
| ATOM | 1949 | CD1 | LEU | A | 273 | 21.923 | 52.966 | 21.914 | 1.00 | 40.75 | C |
| ATOM | 1950 | CD2 | LEU | A | 273 | 21.174 | 55.277 | 21.304 | 1.00 | 39.54 | C |
| ATOM | 1951 | C | LEU | A | 273 | 21.146 | 54.350 | 17.168 | 1.00 | 40.47 | C |
| ATOM | 1952 | O | LEU | A | 273 | 21.742 | 55.331 | 16.747 | 1.00 | 40.46 | O |
| ATOM | 1953 | N | ARG | A | 274 | 21.051 | 53.225 | 16.470 | 1.00 | 41.49 | N |
| ATOM | 1954 | CA | ARG | A | 274 | 21.670 | 53.138 | 15.151 | 1.00 | 43.63 | C |
| ATOM | 1955 | CB | ARG | A | 274 | 21.929 | 51.683 | 14.753 | 1.00 | 44.96 | C |
| ATOM | 1956 | CG | ARG | A | 274 | 22.917 | 50.943 | 15.665 | 1.00 | 51.46 | C |
| ATOM | 1957 | CD | ARG | A | 274 | 23.145 | 49.470 | 15.275 | 1.00 | 60.47 | C |
| ATOM | 1958 | NE | ARG | A | 274 | 23.426 | 49.354 | 13.842 | 1.00 | 66.38 | N |
| ATOM | 1959 | CZ | ARG | A | 274 | 23.511 | 48.212 | 13.172 | 1.00 | 69.93 | C |
| ATOM | 1960 | NH1 | ARG | A | 274 | 23.344 | 47.048 | 13.792 | 1.00 | 71.16 | N |
| ATOM | 1961 | NH2 | ARG | A | 274 | 23.775 | 48.239 | 11.868 | 1.00 | 72.00 | N |
| ATOM | 1962 | C | ARG | A | 274 | 20.784 | 53.826 | 14.117 | 1.00 | 42.18 | C |
| ATOM | 1963 | O | ARG | A | 274 | 19.632 | 53.469 | 13.959 | 1.00 | 42.54 | O |
| ATOM | 1964 | N | PRO | A | 275 | 21.325 | 54.807 | 13.409 | 1.00 | 41.48 | N |
| ATOM | 1965 | CA | PRO | A | 275 | 20.566 | 55.529 | 12.383 | 1.00 | 41.53 | C |
| ATOM | 1966 | CB | PRO | A | 275 | 21.655 | 56.302 | 11.648 | 1.00 | 41.70 | C |
| ATOM | 1967 | CG | PRO | A | 275 | 22.618 | 56.629 | 12.745 | 1.00 | 41.06 | C |
| ATOM | 1968 | CD | PRO | A | 275 | 22.693 | 55.340 | 13.546 | 1.00 | 41.06 | C |
| ATOM | 1969 | C | PRO | A | 275 | 19.784 | 54.624 | 11.429 | 1.00 | 41.71 | C |
| ATOM | 1970 | O | PRO | A | 275 | 18.633 | 54.932 | 11.132 | 1.00 | 39.61 | O |
| ATOM | 1971 | N | SER | A | 276 | 20.393 | 53.516 | 10.993 | 1.00 | 41.90 | N |
| ATOM | 1972 | CA | SER | A | 276 | 19.774 | 52.587 | 10.040 | 1.00 | 42.57 | C |
| ATOM | 1973 | CB | SER | A | 276 | 20.831 | 51.624 | 9.446 | 1.00 | 43.08 | C |
| ATOM | 1974 | OG | SER | A | 276 | 21.290 | 50.683 | 10.419 | 1.00 | 45.84 | O |
| ATOM | 1975 | C | SER | A | 276 | 18.597 | 51.799 | 10.613 | 1.00 | 41.74 | C |
| ATOM | 1976 | O | SER | A | 276 | 17.786 | 51.287 | 9.845 | 1.00 | 42.39 | O |
| ATOM | 1977 | N | ASP | A | 277 | 18.497 | 51.696 | 11.942 | 1.00 | 40.48 | N |
| ATOM | 1978 | CA | ASP | A | 277 | 17.344 | 51.038 | 12.575 | 1.00 | 39.45 | C |
| ATOM | 1979 | CB | ASP | A | 277 | 17.676 | 50.520 | 13.981 | 1.00 | 40.14 | C |
| ATOM | 1980 | CG | ASP | A | 277 | 18.671 | 49.374 | 13.974 | 1.00 | 41.45 | C |
| ATOM | 1981 | OD1 | ASP | A | 277 | 18.697 | 48.577 | 13.010 | 1.00 | 43.47 | O |
| ATOM | 1982 | OD2 | ASP | A | 277 | 19.471 | 49.221 | 14.915 | 1.00 | 43.49 | O |
| ATOM | 1983 | C | ASP | A | 277 | 16.102 | 51.946 | 12.676 | 1.00 | 38.59 | C |
| ATOM | 1984 | O | ASP | A | 277 | 15.010 | 51.486 | 13.014 | 1.00 | 37.61 | O |
| ATOM | 1985 | N | ARG | A | 278 | 16.269 | 53.227 | 12.364 | 1.00 | 37.09 | N |
| ATOM | 1986 | CA | ARG | A | 278 | 15.145 | 54.159 | 12.448 | 1.00 | 35.81 | C |
| ATOM | 1987 | CB | ARG | A | 278 | 15.657 | 55.598 | 12.545 | 1.00 | 34.54 | C |
| ATOM | 1988 | CG | ARG | A | 278 | 16.407 | 55.836 | 13.836 | 1.00 | 34.40 | C |
| ATOM | 1989 | CD | ARG | A | 278 | 17.017 | 57.225 | 13.957 | 1.00 | 35.33 | C |
| ATOM | 1990 | NE | ARG | A | 278 | 18.119 | 57.186 | 14.913 | 1.00 | 35.31 | N |
| ATOM | 1991 | CZ | ARG | A | 278 | 19.163 | 57.996 | 14.913 | 1.00 | 36.13 | C |
| ATOM | 1992 | NH1 | ARG | A | 278 | 19.286 | 58.971 | 14.010 | 1.00 | 34.75 | N |
| ATOM | 1993 | NH2 | ARG | A | 278 | 20.103 | 57.815 | 15.829 | 1.00 | 36.28 | N |
| ATOM | 1994 | C | ARG | A | 278 | 14.223 | 53.983 | 11.243 | 1.00 | 36.08 | C |
| ATOM | 1995 | O | ARG | A | 278 | 14.687 | 53.610 | 10.156 | 1.00 | 36.69 | O |
| ATOM | 1996 | N | PRO | A | 279 | 12.936 | 54.275 | 11.421 | 1.00 | 35.45 | N |
| ATOM | 1997 | CA | PRO | A | 279 | 11.984 | 54.193 | 10.314 | 1.00 | 35.56 | C |
| ATOM | 1998 | CB | PRO | A | 279 | 10.627 | 54.303 | 11.004 | 1.00 | 35.57 | C |
| ATOM | 1999 | CG | PRO | A | 279 | 10.915 | 55.147 | 12.224 | 1.00 | 35.53 | C |
| ATOM | 2000 | CD | PRO | A | 279 | 12.284 | 54.710 | 12.677 | 1.00 | 35.37 | C |
| ATOM | 2001 | C | PRO | A | 279 | 12.174 | 55.322 | 9.311 | 1.00 | 35.53 | C |
| ATOM | 2002 | O | PRO | A | 279 | 12.784 | 56.354 | 9.626 | 1.00 | 35.63 | O |
| ATOM | 2003 | N | THR | A | 280 | 11.661 | 55.107 | 8.101 | 1.00 | 34.84 | N |
| ATOM | 2004 | CA | THR | A | 280 | 11.618 | 56.145 | 7.079 | 1.00 | 34.43 | C |
| ATOM | 2005 | CB | THR | A | 280 | 11.509 | 55.513 | 5.683 | 1.00 | 34.63 | C |
| ATOM | 2006 | OG1 | THR | A | 280 | 10.344 | 54.690 | 5.655 | 1.00 | 34.43 | O |
| ATOM | 2007 | CG2 | THR | A | 280 | 12.712 | 54.555 | 5.379 | 1.00 | 35.88 | C |
| ATOM | 2008 | C | THR | A | 280 | 10.334 | 56.900 | 7.337 | 1.00 | 34.28 | C |
| ATOM | 2009 | O | THR | A | 280 | 9.501 | 56.458 | 8.120 | 1.00 | 33.22 | O |
| ATOM | 2010 | N | PHE | A | 281 | 10.129 | 58.012 | 6.637 | 1.00 | 35.30 | N |
| ATOM | 2011 | CA | PHE | A | 281 | 8.893 | 58.771 | 6.797 | 1.00 | 35.82 | C |
| ATOM | 2012 | CB | PHE | A | 281 | 8.892 | 60.020 | 5.907 | 1.00 | 36.90 | C |
| ATOM | 2013 | CG | PHE | A | 281 | 9.984 | 61.009 | 6.223 | 1.00 | 38.29 | C |
| ATOM | 2014 | CD1 | PHE | A | 281 | 10.332 | 61.300 | 7.536 | 1.00 | 39.19 | C |
| ATOM | 2015 | CE1 | PHE | A | 281 | 11.320 | 62.234 | 7.823 | 1.00 | 39.73 | C |
| ATOM | 2016 | CZ | PHE | A | 281 | 11.968 | 62.874 | 6.810 | 1.00 | 41.57 | C |
| ATOM | 2017 | CE2 | PHE | A | 281 | 11.621 | 62.608 | 5.483 | 1.00 | 43.04 | C |
| ATOM | 2018 | CD2 | PHE | A | 281 | 10.633 | 61.681 | 5.200 | 1.00 | 41.16 | C |
| ATOM | 2019 | C | PHE | A | 281 | 7.690 | 57.894 | 6.477 | 1.00 | 36.17 | C |
| ATOM | 2020 | O | PHE | A | 281 | 6.671 | 57.924 | 7.179 | 1.00 | 35.36 | O |
| ATOM | 2021 | N | GLU | A | 282 | 7.815 | 57.101 | 5.414 | 1.00 | 35.33 | N |
| ATOM | 2022 | CA | GLU | A | 282 | 6.741 | 56.194 | 4.992 | 1.00 | 35.04 | C |
| ATOM | 2023 | CB | GLU | A | 282 | 7.154 | 55.461 | 3.700 | 1.00 | 35.95 | C |
| ATOM | 2024 | CG | GLU | A | 282 | 6.092 | 54.530 | 3.141 | 1.00 | 38.88 | C |
| ATOM | 2025 | CD | GLU | A | 282 | 6.504 | 53.872 | 1.819 | 1.00 | 42.76 | C |
| ATOM | 2026 | OE1 | GLU | A | 282 | 7.654 | 54.056 | 1.362 | 1.00 | 43.67 | O |
| ATOM | 2027 | OE2 | GLU | A | 282 | 5.654 | 53.182 | 1.233 | 1.00 | 43.19 | O |
| ATOM | 2028 | C | GLU | A | 282 | 6.385 | 55.199 | 6.084 | 1.00 | 34.27 | C |
| ATOM | 2029 | O | GLU | A | 282 | 5.209 | 54.986 | 6.378 | 1.00 | 34.51 | O |
| ATOM | 2030 | N | GLU | A | 283 | 7.397 | 54.594 | 6.693 | 1.00 | 34.18 | N |
| ATOM | 2031 | CA | GLU | A | 283 | 7.194 | 53.640 | 7.795 | 1.00 | 34.58 | C |
| ATOM | 2032 | CB | GLU | A | 283 | 8.512 | 53.012 | 8.208 | 1.00 | 35.26 | C |
| ATOM | 2033 | CG | GLU | A | 283 | 9.077 | 52.096 | 7.131 | 1.00 | 38.60 | C |
| ATOM | 2034 | CD | GLU | A | 283 | 10.406 | 51.501 | 7.502 | 1.00 | 40.02 | C |
| ATOM | 2035 | OE1 | GLU | A | 283 | 11.340 | 52.257 | 7.832 | 1.00 | 41.52 | O |
| ATOM | 2036 | OE2 | GLU | A | 283 | 10.517 | 50.266 | 7.435 | 1.00 | 44.14 | O |
| ATOM | 2037 | C | GLU | A | 283 | 6.524 | 54.259 | 9.014 | 1.00 | 33.96 | C |
| ATOM | 2038 | O | GLU | A | 283 | 5.700 | 53.614 | 9.674 | 1.00 | 33.93 | O |
| ATOM | 2039 | N | ILE | A | 284 | 6.859 | 55.517 | 9.298 | 1.00 | 33.26 | N |
| ATOM | 2040 | CA | ILE | A | 284 | 6.204 | 56.233 | 10.401 | 1.00 | 31.63 | C |
| ATOM | 2041 | CB | ILE | A | 284 | 6.889 | 57.590 | 10.650 | 1.00 | 31.52 | C |
| ATOM | 2042 | CG1 | ILE | A | 284 | 8.282 | 57.373 | 11.252 | 1.00 | 29.17 | C |
| ATOM | 2043 | CD1 | ILE | A | 284 | 9.195 | 58.585 | 11.190 | 1.00 | 32.09 | C |
| ATOM | 2044 | CG2 | ILE | A | 284 | 6.002 | 58.489 | 11.602 | 1.00 | 29.23 | C |
| ATOM | 2045 | C | ILE | A | 284 | 4.732 | 56.430 | 10.089 | 1.00 | 31.80 | C |
| ATOM | 2046 | O | ILE | A | 284 | 3.856 | 56.165 | 10.917 | 1.00 | 31.97 | O |
| ATOM | 2047 | N | GLN | A | 285 | 4.451 | 56.917 | 8.886 | 1.00 | 32.64 | N |
| ATOM | 2048 | CA | GLN | A | 285 | 3.070 | 57.204 | 8.515 | 1.00 | 32.77 | C |
| ATOM | 2049 | CB | GLN | A | 285 | 3.022 | 58.099 | 7.280 | 1.00 | 32.86 | C |
| ATOM | 2050 | CG | GLN | A | 285 | 3.373 | 59.566 | 7.613 | 1.00 | 32.93 | C |
| ATOM | 2051 | CD | GLN | A | 285 | 3.056 | 60.507 | 6.485 | 1.00 | 35.30 | C |
| ATOM | 2052 | OE1 | GLN | A | 285 | 3.637 | 60.401 | 5.395 | 1.00 | 34.32 | O |
| ATOM | 2053 | NE2 | GLN | A | 285 | 2.122 | 61.423 | 6.725 | 1.00 | 33.69 | N |
| ATOM | 2054 | C | GLN | A | 285 | 2.239 | 55.948 | 8.334 | 1.00 | 33.74 | C |
| ATOM | 2055 | O | GLN | A | 285 | 1.021 | 55.996 | 8.454 | 1.00 | 34.52 | O |
| ATOM | 2056 | N | ASN | A | 286 | 2.889 | 54.816 | 8.084 | 1.00 | 34.59 | N |
| ATOM | 2057 | CA | ASN | A | 286 | 2.165 | 53.533 | 8.016 | 1.00 | 36.22 | C |
| ATOM | 2058 | CB | ASN | A | 286 | 2.770 | 52.607 | 6.966 | 1.00 | 35.90 | C |
| ATOM | 2059 | CG | ASN | A | 286 | 2.450 | 53.042 | 5.553 | 1.00 | 37.57 | C |
| ATOM | 2060 | OD1 | ASN | A | 286 | 1.397 | 53.611 | 5.283 | 1.00 | 37.74 | O |
| ATOM | 2061 | ND2 | ASN | A | 286 | 3.373 | 52.785 | 4.642 | 1.00 | 39.91 | N |
| ATOM | 2062 | C | ASN | A | 286 | 2.079 | 52.805 | 9.360 | 1.00 | 36.58 | C |
| ATOM | 2063 | O | ASN | A | 286 | 1.432 | 51.767 | 9.466 | 1.00 | 37.11 | O |
| ATOM | 2064 | N | HIS | A | 287 | 2.723 | 53.356 | 10.384 | 1.00 | 36.48 | N |
| ATOM | 2065 | CA | HIS | A | 287 | 2.677 | 52.771 | 11.717 | 1.00 | 36.00 | C |
| ATOM | 2066 | CB | HIS | A | 287 | 3.525 | 53.596 | 12.697 | 1.00 | 35.69 | C |
| ATOM | 2067 | CG | HIS | A | 287 | 3.703 | 52.938 | 14.029 | 1.00 | 33.97 | C |
| ATOM | 2068 | ND1 | HIS | A | 287 | 4.826 | 52.211 | 14.359 | 1.00 | 35.79 | N |
| ATOM | 2069 | CE1 | HIS | A | 287 | 4.706 | 51.749 | 15.592 | 1.00 | 34.10 | C |
| ATOM | 2070 | NE2 | HIS | A | 287 | 3.537 | 52.138 | 16.066 | 1.00 | 36.32 | N |
| ATOM | 2071 | CD2 | HIS | A | 287 | 2.888 | 52.875 | 15.103 | 1.00 | 33.02 | C |
| ATOM | 2072 | C | HIS | A | 287 | 1.238 | 52.724 | 12.223 | 1.00 | 36.98 | C |
| ATOM | 2073 | O | HIS | A | 287 | 0.475 | 53.663 | 11.985 | 1.00 | 36.67 | O |
| ATOM | 2074 | N | PRO | A | 288 | 0.870 | 51.638 | 12.909 | 1.00 | 37.63 | N |
| ATOM | 2075 | CA | PRO | A | 288 | −0.465 | 51.480 | 13.468 | 1.00 | 38.40 | C |
| ATOM | 2076 | CB | PRO | A | 288 | −0.318 | 50.203 | 14.315 | 1.00 | 39.46 | C |
| ATOM | 2077 | CG | PRO | A | 288 | 0.684 | 49.420 | 13.576 | 1.00 | 40.31 | C |
| ATOM | 2078 | CD | PRO | A | 288 | 1.699 | 50.447 | 13.174 | 1.00 | 38.01 | C |
| ATOM | 2079 | C | PRO | A | 288 | −0.936 | 52.652 | 14.325 | 1.00 | 37.78 | C |
| ATOM | 2080 | O | PRO | A | 288 | −2.096 | 53.024 | 14.227 | 1.00 | 38.92 | O |
| ATOM | 2081 | N | TRP | A | 289 | −0.062 | 53.231 | 15.143 | 1.00 | 37.99 | N |
| ATOM | 2082 | CA | TRP | A | 289 | −0.459 | 54.387 | 15.951 | 1.00 | 36.84 | C |
| ATOM | 2083 | CB | TRP | A | 289 | 0.642 | 54.779 | 16.932 | 1.00 | 37.43 | C |
| ATOM | 2084 | CG | TRP | A | 289 | 0.197 | 55.892 | 17.862 | 1.00 | 36.44 | C |
| ATOM | 2085 | CD1 | TRP | A | 289 | −0.601 | 55.776 | 18.969 | 1.00 | 36.79 | C |
| ATOM | 2086 | NE1 | TRP | A | 289 | −0.800 | 57.014 | 19.542 | 1.00 | 35.91 | N |
| ATOM | 2087 | CE2 | TRP | A | 289 | −0.136 | 57.956 | 18.795 | 1.00 | 35.04 | C |
| ATOM | 2088 | CD2 | TRP | A | 289 | 0.500 | 57.281 | 17.730 | 1.00 | 35.44 | C |
| ATOM | 2089 | CE3 | TRP | A | 289 | 1.275 | 58.034 | 16.822 | 1.00 | 34.75 | C |
| ATOM | 2090 | CZ3 | TRP | A | 289 | 1.365 | 59.412 | 17.000 | 1.00 | 33.10 | C |
| ATOM | 2091 | CH2 | TRP | A | 289 | 0.719 | 60.046 | 18.072 | 1.00 | 32.65 | C |
| ATOM | 2092 | CZ2 | TRP | A | 289 | −0.024 | 59.337 | 18.980 | 1.00 | 35.41 | C |
| ATOM | 2093 | C | TRP | A | 289 | −0.886 | 55.598 | 15.102 | 1.00 | 37.02 | C |
| ATOM | 2094 | O | TRP | A | 289 | −1.703 | 56.402 | 15.551 | 1.00 | 36.85 | O |
| ATOM | 2095 | N | MET | A | 290 | −0.375 | 55.704 | 13.875 | 1.00 | 37.67 | N |
| ATOM | 2096 | CA | MET | A | 290 | −0.681 | 56.857 | 13.002 | 1.00 | 39.50 | C |
| ATOM | 2097 | CB | MET | A | 290 | 0.475 | 57.119 | 12.038 | 1.00 | 38.69 | C |
| ATOM | 2098 | CG | MET | A | 290 | 1.770 | 57.552 | 12.737 | 1.00 | 39.97 | C |
| ATOM | 2099 | SD | MET | A | 290 | 2.026 | 59.340 | 12.660 | 1.00 | 43.39 | S |
| ATOM | 2100 | CE | MET | A | 290 | 0.826 | 59.826 | 13.609 | 1.00 | 36.57 | C |
| ATOM | 2101 | C | MET | A | 290 | −1.973 | 56.787 | 12.186 | 1.00 | 41.20 | C |
| ATOM | 2102 | O | MET | A | 290 | −2.269 | 57.703 | 11.397 | 1.00 | 40.58 | O |
| ATOM | 2103 | N | GLN | A | 291 | −2.735 | 55.709 | 12.338 | 1.00 | 43.28 | N |
| ATOM | 2104 | CA | GLN | A | 291 | −3.928 | 55.516 | 11.504 | 1.00 | 45.49 | C |
| ATOM | 2105 | CB | GLN | A | 291 | −4.294 | 54.031 | 11.420 | 1.00 | 46.70 | C |
| ATOM | 2106 | CG | GLN | A | 291 | −3.169 | 53.163 | 10.863 | 1.00 | 52.12 | C |
| ATOM | 2107 | CD | GLN | A | 291 | −2.989 | 53.257 | 9.330 | 1.00 | 58.53 | C |
| ATOM | 2108 | OE1 | GLN | A | 291 | −3.107 | 54.339 | 8.723 | 1.00 | 59.20 | O |
| ATOM | 2109 | NE2 | GLN | A | 291 | −2.674 | 52.113 | 8.708 | 1.00 | 62.36 | N |
| ATOM | 2110 | C | GLN | A | 291 | −5.112 | 56.329 | 12.001 | 1.00 | 45.68 | C |
| ATOM | 2111 | O | GLN | A | 291 | −5.165 | 56.708 | 13.177 | 1.00 | 45.87 | O |
| ATOM | 2112 | N | ASP | A | 292 | −6.064 | 56.590 | 11.100 | 1.00 | 46.07 | N |
| ATOM | 2113 | CA | ASP | A | 292 | −7.305 | 57.331 | 11.410 | 1.00 | 46.57 | C |
| ATOM | 2114 | CB | ASP | A | 292 | −8.195 | 56.556 | 12.391 | 1.00 | 47.72 | C |
| ATOM | 2115 | CG | ASP | A | 292 | −8.324 | 55.099 | 12.018 | 1.00 | 52.03 | C |
| ATOM | 2116 | OD1 | ASP | A | 292 | −8.714 | 54.836 | 10.858 | 1.00 | 54.96 | O |
| ATOM | 2117 | OD2 | ASP | A | 292 | −8.031 | 54.164 | 12.805 | 1.00 | 56.19 | O |
| ATOM | 2118 | C | ASP | A | 292 | −7.071 | 58.739 | 11.961 | 1.00 | 45.48 | C |
| ATOM | 2119 | O | ASP | A | 292 | −7.779 | 59.181 | 12.880 | 1.00 | 44.34 | O |
| ATOM | 2120 | N | VAL | A | 293 | −6.067 | 59.433 | 11.424 | 1.00 | 44.87 | N |
| ATOM | 2121 | CA | VAL | A | 293 | −5.750 | 60.787 | 11.882 | 1.00 | 44.61 | C |
| ATOM | 2122 | CB | VAL | A | 293 | −4.495 | 61.338 | 11.181 | 1.00 | 44.85 | C |
| ATOM | 2123 | CG1 | VAL | A | 293 | −4.791 | 61.662 | 9.735 | 1.00 | 44.77 | C |
| ATOM | 2124 | CG2 | VAL | A | 293 | −3.989 | 62.568 | 11.896 | 1.00 | 43.11 | C |
| ATOM | 2125 | C | VAL | A | 293 | −6.935 | 61.709 | 11.640 | 1.00 | 44.94 | C |
| ATOM | 2126 | O | VAL | A | 293 | −7.658 | 61.532 | 10.653 | 1.00 | 45.74 | O |
| ATOM | 2127 | N | LEU | A | 294 | −7.149 | 62.668 | 12.538 | 1.00 | 44.60 | N |
| ATOM | 2128 | CA | LEU | A | 294 | −8.209 | 63.650 | 12.354 | 1.00 | 44.83 | C |
| ATOM | 2129 | CB | LEU | A | 294 | −8.489 | 64.416 | 13.641 | 1.00 | 43.88 | C |
| ATOM | 2130 | CG | LEU | A | 294 | −9.009 | 63.721 | 14.886 | 1.00 | 44.35 | C |
| ATOM | 2131 | CD1 | LEU | A | 294 | −9.118 | 64.760 | 15.972 | 1.00 | 42.61 | C |
| ATOM | 2132 | CD2 | LEU | A | 294 | −10.337 | 63.036 | 14.632 | 1.00 | 45.06 | C |
| ATOM | 2133 | C | LEU | A | 294 | −7.763 | 64.655 | 11.312 | 1.00 | 45.35 | C |
| ATOM | 2134 | O | LEU | A | 294 | −6.570 | 64.888 | 11.142 | 1.00 | 45.48 | O |
| ATOM | 2135 | N | LEU | A | 295 | −8.728 | 65.266 | 10.629 | 1.00 | 46.10 | N |
| ATOM | 2136 | CA | LEU | A | 295 | −8.444 | 66.358 | 9.712 | 1.00 | 46.34 | C |
| ATOM | 2137 | CB | LEU | A | 295 | −9.645 | 66.586 | 8.790 | 1.00 | 47.42 | C |
| ATOM | 2138 | CG | LEU | A | 295 | −9.552 | 65.968 | 7.380 | 1.00 | 50.24 | C |
| ATOM | 2139 | CD1 | LEU | A | 295 | −9.352 | 64.460 | 7.415 | 1.00 | 51.66 | C |
| ATOM | 2140 | CD2 | LEU | A | 295 | −10.812 | 66.288 | 6.595 | 1.00 | 54.37 | C |
| ATOM | 2141 | C | LEU | A | 295 | −8.123 | 67.612 | 10.527 | 1.00 | 46.02 | C |
| ATOM | 2142 | O | LEU | A | 295 | −8.531 | 67.723 | 11.693 | 1.00 | 44.80 | O |
| ATOM | 2143 | N | PRO | A | 296 | −7.366 | 68.544 | 9.955 | 1.00 | 46.46 | N |
| ATOM | 2144 | CA | PRO | A | 296 | −7.048 | 69.790 | 10.658 | 1.00 | 47.09 | C |
| ATOM | 2145 | CB | PRO | A | 296 | −6.405 | 70.633 | 9.561 | 1.00 | 46.93 | C |
| ATOM | 2146 | CG | PRO | A | 296 | −5.698 | 69.609 | 8.741 | 1.00 | 45.84 | C |
| ATOM | 2147 | CD | PRO | A | 296 | −6.708 | 68.496 | 8.638 | 1.00 | 46.93 | C |
| ATOM | 2148 | C | PRO | A | 296 | −8.282 | 70.465 | 11.266 | 1.00 | 48.28 | C |
| ATOM | 2149 | O | PRO | A | 296 | −8.280 | 70.739 | 12.474 | 1.00 | 47.68 | O |
| ATOM | 2150 | N | GLN | A | 297 | −9.335 | 70.684 | 10.480 | 1.00 | 50.01 | N |
| ATOM | 2151 | CA | GLN | A | 297 | −10.537 | 71.328 | 11.022 | 1.00 | 51.78 | C |
| ATOM | 2152 | CG | GLN | A | 297 | −11.572 | 71.636 | 9.933 | 1.00 | 52.87 | C |
| ATOM | 2153 | CG | GLN | A | 297 | −12.552 | 72.781 | 10.298 | 1.00 | 55.96 | C |
| ATOM | 2154 | CD | GLN | A | 297 | −11.858 | 74.122 | 10.632 | 1.00 | 60.05 | C |
| ATOM | 2155 | OE1 | GLN | A | 297 | −11.221 | 74.739 | 9.765 | 1.00 | 62.29 | O |
| ATOM | 2156 | NE2 | GLN | A | 297 | −11.992 | 74.570 | 11.884 | 1.00 | 60.16 | N |
| ATOM | 2157 | C | GLN | A | 297 | −11.175 | 70.550 | 12.181 | 1.00 | 51.71 | C |
| ATOM | 2158 | O | GLN | A | 297 | −11.536 | 71.140 | 13.201 | 1.00 | 52.21 | O |
| ATOM | 2159 | N | GLU | A | 298 | −11.292 | 69.234 | 12.034 | 1.00 | 51.63 | N |
| ATOM | 2160 | CA | GLU | A | 298 | −11.819 | 68.391 | 13.108 | 1.00 | 51.67 | C |
| ATOM | 2161 | CB | GLU | A | 298 | −11.714 | 66.922 | 12.736 | 1.00 | 52.61 | C |
| ATOM | 2162 | CG | GLU | A | 298 | −12.716 | 66.406 | 11.732 | 1.00 | 56.45 | C |
| ATOM | 2163 | CD | GLU | A | 298 | −12.568 | 64.908 | 11.552 | 1.00 | 60.37 | C |
| ATOM | 2164 | OE1 | GLU | A | 298 | −11.606 | 64.480 | 10.874 | 1.00 | 61.21 | O |
| ATOM | 2165 | OE2 | GLU | A | 298 | −13.403 | 64.160 | 12.112 | 1.00 | 63.66 | O |
| ATOM | 2166 | C | GLU | A | 298 | −10.991 | 68.586 | 14.372 | 1.00 | 50.78 | C |
| ATOM | 2167 | O | GLU | A | 298 | −11.523 | 68.666 | 15.490 | 1.00 | 50.03 | O |
| ATOM | 2168 | N | THR | A | 299 | −9.676 | 68.624 | 14.186 | 1.00 | 49.28 | N |
| ATOM | 2169 | CA | THR | A | 299 | −8.756 | 68.812 | 15.291 | 1.00 | 48.32 | C |
| ATOM | 2170 | CB | THR | A | 299 | −7.310 | 68.855 | 14.781 | 1.00 | 48.02 | C |
| ATOM | 2171 | OG1 | THR | A | 299 | −7.007 | 67.636 | 14.096 | 1.00 | 45.02 | O |
| ATOM | 2172 | CG2 | THR | A | 299 | −6.324 | 68.910 | 15.951 | 1.00 | 47.18 | C |
| ATOM | 2173 | C | THR | A | 299 | −9.072 | 70.095 | 16.040 | 1.00 | 48.76 | C |
| ATOM | 2174 | O | THR | A | 299 | −9.135 | 70.101 | 17.268 | 1.00 | 47.62 | O |
| ATOM | 2175 | N | ALA | A | 300 | −9.252 | 71.181 | 15.293 | 1.00 | 49.46 | N |
| ATOM | 2176 | CA | ALA | A | 300 | −9.540 | 72.468 | 15.887 | 1.00 | 50.94 | C |
| ATOM | 2177 | CB | ALA | A | 300 | −9.541 | 73.556 | 14.820 | 1.00 | 50.83 | C |
| ATOM | 2178 | C | ALA | A | 300 | −10.875 | 72.438 | 16.664 | 1.00 | 51.99 | C |
| ATOM | 2179 | O | ALA | A | 300 | −10.961 | 72.940 | 17.793 | 1.00 | 51.96 | O |
| ATOM | 2180 | N | GLU | A | 301 | −11.896 | 71.832 | 16.064 | 1.00 | 53.05 | N |
| ATOM | 2181 | CA | GLU | A | 301 | −13.218 | 71.757 | 16.689 | 1.00 | 54.49 | C |
| ATOM | 2182 | CB | GLU | A | 301 | −14.220 | 71.094 | 15.754 | 1.00 | 55.03 | C |
| ATOM | 2183 | CG | GLU | A | 301 | −14.926 | 72.073 | 14.831 | 1.00 | 58.47 | C |
| ATOM | 2184 | CD | GLU | A | 301 | −15.129 | 71.518 | 13.429 | 1.00 | 61.94 | C |
| ATOM | 2185 | OE1 | GLU | A | 301 | −15.418 | 70.303 | 13.287 | 1.00 | 62.49 | O |
| ATOM | 2186 | OE2 | GLU | A | 301 | −15.006 | 72.306 | 12.459 | 1.00 | 64.87 | O |
| ATOM | 2187 | C | GLU | A | 301 | −13.177 | 71.018 | 18.026 | 1.00 | 54.40 | C |
| ATOM | 2188 | O | GLU | A | 301 | −13.652 | 71.536 | 19.048 | 1.00 | 54.62 | O |
| ATOM | 2189 | N | ILE | A | 302 | −12.581 | 69.826 | 18.011 | 1.00 | 54.14 | N |
| ATOM | 2190 | CA | ILE | A | 302 | −12.487 | 68.970 | 19.196 | 1.00 | 53.52 | C |
| ATOM | 2191 | CB | ILE | A | 302 | −12.124 | 67.529 | 18.791 | 1.00 | 53.66 | C |
| ATOM | 2192 | CG1 | ILE | A | 302 | −13.150 | 66.981 | 17.795 | 1.00 | 53.26 | C |
| ATOM | 2193 | CD1 | ILE | A | 302 | −12.813 | 65.609 | 17.254 | 1.00 | 52.59 | C |
| ATOM | 2194 | CG2 | ILE | A | 302 | −12.047 | 66.624 | 20.026 | 1.00 | 53.65 | C |
| ATOM | 2195 | C | ILE | A | 302 | −11.496 | 69.462 | 20.246 | 1.00 | 53.50 | C |
| ATOM | 2196 | O | ILE | A | 302 | −11.800 | 69.422 | 21.440 | 1.00 | 53.33 | O |
| ATOM | 2197 | N | HIS | A | 303 | −10.322 | 69.937 | 19.822 | 1.00 | 53.16 | N |
| ATOM | 2198 | CA | HIS | A | 303 | −9.258 | 70.220 | 20.793 | 1.00 | 53.02 | C |
| ATOM | 2199 | CB | HIS | A | 303 | −8.018 | 69.390 | 20.459 | 1.00 | 51.63 | C |
| ATOM | 2200 | CG | HIS | A | 303 | −8.212 | 67.926 | 20.680 | 1.00 | 47.34 | C |
| ATOM | 2201 | ND1 | HIS | A | 303 | −8.396 | 67.043 | 19.640 | 1.00 | 45.24 | N |
| ATOM | 2202 | CE1 | HIS | A | 303 | −8.540 | 65.822 | 20.119 | 1.00 | 42.60 | C |
| ATOM | 2203 | NE2 | HIS | A | 303 | −8.456 | 65.883 | 21.437 | 1.00 | 42.67 | N |
| ATOM | 2204 | CD2 | HIS | A | 303 | −8.251 | 67.188 | 21.815 | 1.00 | 43.90 | C |
| ATOM | 2205 | C | HIS | A | 303 | −8.861 | 71.671 | 20.960 | 1.00 | 54.55 | C |
| ATOM | 2206 | O | HIS | A | 303 | −8.265 | 72.036 | 21.979 | 1.00 | 54.31 | O |
| ATOM | 2207 | N | LEU | A | 304 | −9.168 | 72.500 | 19.966 | 1.00 | 56.55 | N |
| ATOM | 2208 | CA | LEU | A | 304 | −8.696 | 73.876 | 19.999 | 1.00 | 59.36 | C |
| ATOM | 2209 | CB | LEU | A | 304 | −7.967 | 74.211 | 18.701 | 1.00 | 58.46 | C |
| ATOM | 2210 | CG | LEU | A | 304 | −6.479 | 73.870 | 18.549 | 1.00 | 58.16 | C |
| ATOM | 2211 | CD1 | LEU | A | 304 | −6.026 | 72.669 | 19.390 | 1.00 | 55.35 | C |
| ATOM | 2212 | CD2 | LEU | A | 304 | −6.160 | 73.653 | 17.061 | 1.00 | 56.12 | C |
| ATOM | 2213 | C | LEU | A | 304 | −9.832 | 74.873 | 20.273 | 1.00 | 62.12 | C |
| ATOM | 2214 | O | LEU | A | 304 | −9.586 | 76.067 | 20.431 | 1.00 | 62.18 | O |
| ATOM | 2215 | N | HIS | A | 305 | −11.061 | 74.361 | 20.340 | 1.00 | 65.89 | N |
| ATOM | 2216 | CA | HIS | A | 305 | −12.278 | 75.150 | 20.571 | 1.00 | 69.84 | C |
| ATOM | 2217 | CB | HIS | A | 305 | −12.201 | 75.963 | 21.884 | 1.00 | 70.73 | C |
| ATOM | 2218 | CG | HIS | A | 305 | −11.780 | 75.138 | 23.069 | 1.00 | 74.80 | C |
| ATOM | 2219 | ND1 | HIS | A | 305 | −12.611 | 74.206 | 23.664 | 1.00 | 77.77 | N |
| ATOM | 2220 | CE1 | HIS | A | 305 | −11.976 | 73.629 | 24.674 | 1.00 | 78.93 | C |
| ATOM | 2221 | NE2 | HIS | A | 305 | −10.760 | 74.149 | 24.753 | 1.00 | 79.02 | N |
| ATOM | 2222 | CD2 | HIS | A | 305 | −10.611 | 75.093 | 23.760 | 1.00 | 77.72 | C |
| ATOM | 2223 | C | HIS | A | 305 | −12.591 | 76.047 | 19.382 | 1.00 | 71.35 | C |
| ATOM | 2224 | O | HIS | A | 305 | −12.458 | 77.272 | 19.463 | 1.00 | 72.07 | O |
| ATOM | 2225 | N | SER | A | 306 | −12.998 | 75.426 | 18.275 | 1.00 | 73.04 | N |
| ATOM | 2226 | CA | SER | A | 306 | −13.372 | 76.161 | 17.066 | 1.00 | 74.54 | C |
| ATOM | 2227 | CB | SER | A | 306 | −12.563 | 75.685 | 15.850 | 1.00 | 74.28 | C |
| ATOM | 2228 | OG | SER | A | 306 | −11.270 | 76.309 | 15.843 | 1.00 | 74.68 | O |
| ATOM | 2229 | C | SER | A | 306 | −14.878 | 76.061 | 16.804 | 1.00 | 75.41 | C |
| ATOM | 2230 | O | SER | A | 306 | −15.588 | 77.080 | 16.858 | 1.00 | 76.03 | O |
| ATOM | 2231 | OXT | SER | A | 306 | −15.397 | 74.966 | 16.542 | 1.00 | 75.98 | O |
| ATOM | 2232 | N3 | IMD | I | 1 | 8.128 | 71.298 | 26.439 | 1.00 | 62.13 | N |
| ATOM | 2233 | C4 | IMD | I | 1 | 8.441 | 71.428 | 27.755 | 1.00 | 62.64 | C |
| ATOM | 2234 | C5 | IMD | I | 1 | 7.731 | 72.513 | 28.267 | 1.00 | 61.10 | C |
| ATOM | 2235 | C2 | IMD | I | 1 | 7.245 | 72.276 | 26.125 | 1.00 | 61.77 | C |
| ATOM | 2236 | N1 | IMD | I | 1 | 7.001 | 73.016 | 27.242 | 1.00 | 61.00 | N |
| ATOM | 2237 | O | HOH | W | 1 | −0.732 | 54.528 | 9.728 | 1.00 | 45.36 | O |
| ATOM | 2238 | O | HOH | W | 2 | 19.630 | 58.716 | 6.576 | 1.00 | 43.01 | O |
| ATOM | 2239 | O | HOH | W | 3 | 0.310 | 61.264 | 2.849 | 1.00 | 32.73 | O |
| ATOM | 2240 | O | HOH | W | 4 | 18.440 | 64.206 | 21.527 | 1.00 | 32.96 | O |
| ATOM | 2241 | O | HOH | W | 5 | 12.988 | 80.668 | 8.424 | 1.00 | 39.01 | O |
| ATOM | 2242 | O | HOH | W | 6 | −1.368 | 51.617 | 30.489 | 1.00 | 40.35 | O |
| ATOM | 2243 | O | HOH | W | 7 | 16.488 | 75.633 | 10.896 | 1.00 | 39.22 | O |
| ATOM | 2244 | O | HOH | W | 8 | 22.715 | 62.695 | 4.286 | 1.00 | 41.65 | O |
| ATOM | 2245 | O | HOH | W | 9 | 15.546 | 67.975 | 9.969 | 1.00 | 34.80 | O |
| ATOM | 2246 | O | HOH | W | 10 | 9.873 | 57.733 | 3.200 | 1.00 | 34.66 | O |
| ATOM | 2247 | O | HOH | W | 11 | 22.041 | 77.197 | 8.223 | 1.00 | 59.58 | O |
| ATOM | 2248 | O | HOH | W | 12 | 13.921 | 68.295 | 7.801 | 1.00 | 43.48 | O |
| ATOM | 2249 | O | HOH | W | 13 | −2.001 | 49.454 | 29.335 | 1.00 | 40.96 | O |
| ATOM | 2250 | O | HOH | W | 14 | 22.261 | 59.914 | 10.882 | 1.00 | 37.32 | O |
| ATOM | 2251 | O | HOH | W | 15 | 19.419 | 50.734 | 16.966 | 1.00 | 40.82 | O |
| ATOM | 2252 | O | HOH | W | 16 | 15.338 | 57.159 | 9.022 | 1.00 | 39.03 | O |
| ATOM | 2253 | O | HOH | W | 17 | 17.961 | 66.549 | 9.882 | 1.00 | 39.50 | O |
| ATOM | 2254 | O | HOH | W | 18 | 4.818 | 76.341 | 0.545 | 1.00 | 44.94 | O |
| ATOM | 2255 | O | HOH | W | 19 | 8.855 | 79.196 | 7.518 | 1.00 | 39.17 | O |
| ATOM | 2256 | O | HOH | W | 20 | 17.072 | 54.130 | 21.844 | 1.00 | 43.20 | O |
| ATOM | 2257 | O | HOH | W | 21 | 1.325 | 69.587 | 7.110 | 1.00 | 36.36 | O |
| ATOM | 2258 | O | HOH | W | 22 | 8.150 | 61.656 | 1.220 | 1.00 | 40.26 | O |
| ATOM | 2259 | O | HOH | W | 23 | −4.435 | 66.666 | 10.979 | 1.00 | 43.16 | O |
| ATOM | 2260 | O | HOH | W | 24 | 10.513 | 80.713 | 9.117 | 1.00 | 42.28 | O |
| ATOM | 2261 | O | HOH | W | 25 | 15.497 | 65.164 | 24.557 | 1.00 | 34.79 | O |
| ATOM | 2262 | O | HOH | W | 26 | 9.900 | 52.831 | 3.589 | 1.00 | 42.40 | O |
| ATOM | 2263 | O | HOH | W | 27 | −0.200 | 71.719 | 8.387 | 1.00 | 41.34 | O |
| ATOM | 2264 | O | HOH | W | 28 | −7.398 | 59.982 | 15.551 | 1.00 | 40.20 | O |
| ATOM | 2265 | O | HOH | W | 29 | 3.492 | 81.322 | 21.013 | 1.00 | 47.98 | O |
| ATOM | 2266 | O | HOH | W | 30 | −4.714 | 67.425 | 25.026 | 1.00 | 43.45 | O |
| ATOM | 2267 | O | HOH | W | 31 | 15.251 | 68.122 | 12.673 | 1.00 | 36.68 | O |
| ATOM | 2268 | O | HOH | W | 32 | −5.709 | 62.260 | 15.119 | 1.00 | 39.90 | O |
| ATOM | 2269 | O | HOH | W | 33 | 4.553 | 83.955 | 11.446 | 1.00 | 45.99 | O |
| ATOM | 2270 | O | HOH | W | 34 | 18.791 | 57.169 | 28.057 | 1.00 | 43.68 | O |
| ATOM | 2271 | O | HOH | W | 35 | 18.231 | 65.464 | 14.872 | 1.00 | 37.87 | O |
| ATOM | 2272 | O | HOH | W | 36 | 8.971 | 53.789 | 30.860 | 1.00 | 43.70 | O |
| ATOM | 2273 | O | HOH | W | 37 | 5.180 | 50.983 | 9.900 | 1.00 | 39.96 | O |
| ATOM | 2274 | O | HOH | W | 38 | −4.081 | 60.211 | 25.479 | 1.00 | 43.04 | O |
| ATOM | 2275 | O | HOH | W | 39 | −1.650 | 50.298 | 24.953 | 1.00 | 50.05 | O |
| ATOM | 2276 | O | HOH | W | 40 | −0.323 | 79.686 | 2.181 | 1.00 | 64.08 | O |
| ATOM | 2277 | O | HOH | W | 41 | −4.014 | 58.332 | 9.232 | 1.00 | 45.77 | O |
| ATOM | 2278 | O | HOH | W | 42 | 10.273 | 50.306 | 18.899 | 1.00 | 43.91 | O |
| ATOM | 2279 | O | HOH | W | 43 | 16.890 | 54.883 | 8.955 | 1.00 | 43.73 | O |
| ATOM | 2280 | O | HOH | W | 44 | 3.730 | 65.993 | 2.097 | 1.00 | 44.04 | O |
| ATOM | 2281 | O | HOH | W | 45 | 23.972 | 70.563 | 2.275 | 1.00 | 40.95 | O |
| ATOM | 2282 | O | HOH | W | 46 | 24.633 | 58.602 | 10.052 | 1.00 | 42.68 | O |
| ATOM | 2283 | O | HOH | W | 47 | 19.828 | 61.618 | 4.358 | 1.00 | 51.38 | O |
| ATOM | 2284 | O | HOH | W | 48 | 22.517 | 90.823 | 15.952 | 1.00 | 70.96 | O |
| ATOM | 2285 | O | HOH | W | 49 | 29.354 | 60.921 | 3.167 | 1.00 | 57.58 | O |
| ATOM | 2286 | O | HOH | W | 50 | 11.468 | 82.369 | 12.289 | 1.00 | 50.02 | O |
| ATOM | 2287 | O | HOH | W | 51 | 24.772 | 62.519 | −4.121 | 1.00 | 45.22 | O |
| ATOM | 2288 | O | HOH | W | 52 | 3.211 | 68.554 | 31.582 | 1.00 | 69.57 | O |
| ATOM | 2289 | O | HOH | W | 53 | 7.936 | 50.002 | 23.124 | 1.00 | 47.40 | O |
| ATOM | 2290 | O | HOH | W | 54 | 15.587 | 71.212 | 17.046 | 1.00 | 52.35 | O |
| ATOM | 2291 | O | HOH | W | 55 | 15.884 | 79.008 | −3.580 | 1.00 | 56.72 | O |
| ATOM | 2292 | O | HOH | W | 56 | 25.279 | 56.110 | 10.230 | 1.00 | 44.21 | O |
| ATOM | 2293 | O | HOH | W | 57 | 12.514 | 58.767 | 4.837 | 1.00 | 52.23 | O |
| ATOM | 2294 | O | HOH | W | 58 | 1.688 | 78.234 | 4.543 | 1.00 | 43.74 | O |
| ATOM | 2295 | O | HOH | W | 59 | 9.018 | 82.803 | 11.168 | 1.00 | 50.76 | O |
| ATOM | 2296 | O | HOH | W | 60 | −0.217 | 85.742 | 6.096 | 1.00 | 53.93 | O |
| ATOM | 2297 | O | HOH | W | 61 | −2.930 | 82.309 | 21.772 | 1.00 | 58.06 | O |
| ATOM | 2298 | O | HOH | W | 62 | 5.504 | 51.225 | 5.130 | 1.00 | 48.90 | O |
| ATOM | 2299 | O | HOH | W | 63 | 20.076 | 54.469 | 7.350 | 1.00 | 61.18 | O |
| ATOM | 2300 | O | HOH | W | 64 | 5.722 | 68.809 | −1.934 | 1.00 | 59.42 | O |
| ATOM | 2301 | O | HOH | W | 65 | 27.882 | 66.292 | −1.512 | 1.00 | 65.79 | O |
| ATOM | 2302 | O | HOH | W | 66 | 19.676 | 72.153 | 23.229 | 1.00 | 61.17 | O |
| ATOM | 2303 | O | HOH | W | 67 | −5.501 | 71.414 | 5.301 | 1.00 | 61.16 | O |
| ATOM | 2304 | O | HOH | W | 68 | 15.016 | 58.056 | 6.473 | 1.00 | 49.90 | O |
| ATOM | 2305 | O | HOH | W | 69 | −2.012 | 55.730 | 6.130 | 1.00 | 56.99 | O |
| ATOM | 2306 | O | HOH | W | 70 | −9.447 | 70.188 | 7.682 | 1.00 | 57.22 | O |
| ATOM | 2307 | O | HOH | W | 71 | 2.484 | 55.120 | −0.038 | 1.00 | 49.25 | O |
| ATOM | 2308 | O | HOH | W | 72 | −7.908 | 59.237 | 8.479 | 1.00 | 65.73 | O |
| ATOM | 2309 | O | HOH | W | 73 | 22.353 | 73.255 | 11.764 | 1.00 | 60.74 | O |
| ATOM | 2310 | O | HOH | W | 74 | 19.477 | 67.324 | 11.857 | 1.00 | 50.67 | O |
| ATOM | 2311 | O | HOH | W | 75 | 14.506 | 47.970 | 13.280 | 1.00 | 62.36 | O |
| ATOM | 2312 | O | HOH | W | 76 | 16.862 | 47.807 | 8.768 | 1.00 | 52.55 | O |
| ATOM | 2313 | O | HOH | W | 77 | 13.313 | 53.083 | 32.098 | 1.00 | 54.43 | O |
| ATOM | 2314 | O | HOH | W | 78 | 17.503 | 50.798 | 19.041 | 1.00 | 55.72 | O |
| ATOM | 2315 | O | HOH | W | 79 | −12.736 | 62.094 | 18.189 | 1.00 | 53.56 | O |
| ATOM | 2316 | O | HOH | W | 80 | 33.908 | 62.979 | 10.712 | 1.00 | 63.79 | O |
| ATOM | 2317 | O | HOH | W | 81 | −6.870 | 60.605 | 22.628 | 1.00 | 49.67 | O |
| ATOM | 2318 | O | HOH | W | 82 | 9.987 | 47.582 | 14.046 | 1.00 | 66.52 | O |
| ATOM | 2319 | O | HOH | W | 83 | 23.183 | 73.287 | 2.510 | 1.00 | 52.15 | O |
| ATOM | 2320 | O | HOH | W | 84 | 27.578 | 55.770 | 9.060 | 1.00 | 63.00 | O |
| ATOM | 2321 | O | HOH | W | 85 | 5.576 | 82.970 | −1.748 | 1.00 | 62.58 | O |
| ATOM | 2322 | O | HOH | W | 86 | −0.509 | 84.330 | 3.857 | 1.00 | 59.32 | O |
| ATOM | 2323 | O | HOH | W | 87 | 13.665 | 91.473 | −3.552 | 1.00 | 61.08 | O |
| ATOM | 2324 | O | HOH | W | 88 | −2.861 | 75.397 | −2.038 | 1.00 | 63.22 | O |
| ATOM | 2325 | O | HOH | W | 89 | 10.204 | 73.979 | 29.790 | 1.00 | 66.32 | O |
| ATOM | 2326 | O | HOH | W | 90 | 20.070 | 78.983 | 6.971 | 1.00 | 67.67 | O |
| ATOM | 2327 | O | HOH | W | 91 | 17.169 | 77.347 | 21.910 | 1.00 | 65.17 | O |
| ATOM | 2328 | O | HOH | W | 92 | −2.870 | 53.045 | 18.163 | 1.00 | 59.47 | O |
| ATOM | 2329 | O | HOH | W | 93 | 11.627 | 71.628 | 23.440 | 1.00 | 63.19 | O |
| ATOM | 2330 | O | HOH | W | 94 | 8.310 | 74.960 | −2.678 | 1.00 | 55.47 | O |
| ATOM | 2331 | O | HOH | W | 95 | −12.002 | 78.851 | 4.304 | 1.00 | 58.94 | O |
| ATOM | 2332 | O | HOH | W | 96 | 5.566 | 49.157 | 22.796 | 1.00 | 51.78 | O |
| ATOM | 2333 | O | HOH | W | 97 | 31.358 | 61.478 | 0.597 | 1.00 | 66.61 | O |
| ATOM | 2334 | O | HOH | W | 98 | 24.035 | 64.093 | −2.091 | 1.00 | 47.25 | O |
| ATOM | 2335 | O | HOH | W | 99 | 11.294 | 69.111 | 34.295 | 1.00 | 70.13 | O |
| ATOM | 2336 | O | HOH | W | 100 | 18.999 | 64.123 | −2.168 | 1.00 | 62.14 | O |
| ATOM | 2337 | O | HOH | W | 101 | −9.739 | 61.493 | 7.698 | 1.00 | 82.68 | O |
| ATOM | 2338 | O | HOH | W | 102 | 22.435 | 52.025 | 25.439 | 1.00 | 54.62 | O |
| ATOM | 2339 | O | HOH | W | 103 | 5.045 | 49.276 | 12.114 | 1.00 | 55.83 | O |
| ATOM | 2340 | O | HOH | W | 104 | −3.965 | 50.524 | 12.224 | 1.00 | 62.13 | O |
| ATOM | 2341 | O | HOH | W | 105 | 13.472 | 75.945 | 26.250 | 1.00 | 61.94 | O |
| ATOM | 2342 | O | HOH | W | 106 | 15.560 | 72.156 | 26.297 | 1.00 | 58.39 | O |
| ATOM | 2343 | O | HOH | W | 107 | −0.195 | 96.034 | 19.635 | 1.00 | 69.60 | O |
| ATOM | 2344 | O | HOH | W | 108 | 1.243 | 88.090 | −4.031 | 1.00 | 62.22 | O |
| ATOM | 2345 | O | HOH | W | 109 | 19.973 | 83.759 | 20.585 | 1.00 | 71.41 | O |
| ATOM | 2346 | O | HOH | W | 110 | −8.152 | 73.486 | 8.288 | 1.00 | 53.47 | O |
| ATOM | 2347 | O | HOH | W | 111 | 23.420 | 81.722 | 9.233 | 1.00 | 71.36 | O |
| ATOM | 2348 | O | HOH | W | 112 | 1.596 | 82.691 | −0.096 | 1.00 | 71.76 | O |
| ATOM | 2349 | O | HOH | W | 113 | 5.657 | 56.059 | −1.336 | 1.00 | 64.94 | O |
| ATOM | 2350 | O | HOH | W | 114 | 13.967 | 51.575 | 8.374 | 1.00 | 51.56 | O |
| ATOM | 2351 | O | HOH | W | 115 | 12.416 | 78.389 | 25.200 | 1.00 | 66.19 | O |
| ATOM | 2352 | O | HOH | W | 116 | 17.235 | 83.392 | 11.447 | 1.00 | 52.25 | O |
| ATOM | 2353 | O | HOH | W | 117 | 14.767 | 52.852 | 21.314 | 1.00 | 47.79 | O |
| ATOM | 2354 | O | HOH | W | 118 | 19.075 | 60.231 | −4.028 | 1.00 | 64.68 | O |
| ATOM | 2355 | O | HOH | W | 119 | 25.476 | 66.800 | 28.823 | 1.00 | 55.96 | O |
| ATOM | 2356 | O | HOH | W | 120 | 4.473 | 70.021 | 30.020 | 1.00 | 56.80 | O |
| ATOM | 2357 | O | HOH | W | 121 | 8.400 | 80.051 | 18.580 | 1.00 | 47.86 | O |
| ATOM | 2358 | O | HOH | W | 122 | −0.274 | 81.374 | 6.467 | 1.00 | 70.59 | O |
| ATOM | 2359 | O | HOH | W | 123 | 8.016 | 51.083 | 3.826 | 1.00 | 50.11 | O |
| ATOM | 2360 | O | HOH | W | 124 | −5.762 | 55.323 | 8.603 | 1.00 | 59.77 | O |
| ATOM | 2361 | O | HOH | W | 125 | 24.801 | 94.210 | −1.115 | 1.00 | 67.33 | O |
| ATOM | 2362 | O | HOH | W | 126 | 9.710 | 48.669 | 26.328 | 1.00 | 63.06 | O |
| ATOM | 2363 | O | HOH | W | 127 | 8.684 | 99.063 | 14.167 | 1.00 | 63.47 | O |
| ATOM | 2364 | O | HOH | W | 128 | 19.451 | 83.648 | 8.511 | 1.00 | 51.41 | O |
| ATOM | 2365 | O | HOH | W | 129 | −10.889 | 61.955 | 10.215 | 1.00 | 55.28 | O |
| ATOM | 2366 | O | HOH | W | 130 | −4.253 | 61.866 | 27.652 | 1.00 | 61.67 | O |
| ATOM | 2367 | O | HOH | W | 131 | 27.030 | 90.340 | 3.848 | 1.00 | 80.85 | O |
| ATOM | 2368 | O | HOH | W | 132 | 10.977 | 87.131 | 22.623 | 1.00 | 65.06 | O |
| ATOM | 2369 | O | HOH | W | 133 | 14.634 | 65.394 | −2.521 | 1.00 | 56.18 | O |
| ATOM | 2370 | O | HOH | W | 134 | −3.405 | 52.808 | 20.692 | 1.00 | 57.93 | O |
| ATOM | 2371 | O | HOH | W | 135 | −5.420 | 55.451 | 15.525 | 1.00 | 51.90 | O |
| ATOM | 2372 | O | HOH | W | 136 | 8.056 | 79.671 | 22.675 | 1.00 | 59.54 | O |
| ATOM | 2373 | O | HOH | W | 137 | 28.392 | 57.786 | 4.755 | 1.00 | 78.57 | O |
| ATOM | 2374 | O | HOH | W | 138 | 18.312 | 99.689 | 9.767 | 1.00 | 61.28 | O |
| ATOM | 2375 | O | HOH | W | 139 | 33.446 | 63.253 | 17.723 | 1.00 | 59.53 | O |
| ATOM | 2376 | O | HOH | W | 140 | 24.283 | 56.206 | 17.474 | 1.00 | 54.00 | O |
| ATOM | 2377 | O | HOH | W | 141 | 16.808 | 50.392 | 32.500 | 1.00 | 57.59 | O |
| ATOM | 2378 | O | HOH | W | 142 | 15.746 | 83.812 | −7.461 | 1.00 | 64.85 | O |
| ATOM | 2379 | O | HOH | W | 143 | −7.082 | 94.424 | −2.169 | 1.00 | 67.76 | O |
| ATOM | 2380 | O | HOH | W | 144 | 13.631 | 49.312 | 10.749 | 1.00 | 54.19 | O |
| ATOM | 2381 | O | HOH | W | 145 | 30.247 | 61.193 | 23.440 | 1.00 | 71.27 | O |
| ATOM | 2382 | O | HOH | W | 146 | 13.010 | 80.075 | −6.528 | 1.00 | 68.99 | O |

TABLE 2

| HEADER | ---- | XX-XXX-XX xxxx |
|---|---|---|
| COMPND | --- | |
| REMARK | 3 | |

| REMARK | 3 | REFINEMENT. | |
|---|---|---|---|
| REMARK | 3 | PROGRAM | REFMAC 5.1.21 |
| REMARK | 3 | AUTHORS | MURSHUDOV, VAGIN, DODSON |
| REMARK | 3 | | |

| REMARK | 3 | REFINEMENT TARGET MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | 2.03 |
| REMARK | 3 | RESOLUTION RANGE LOW | (ANGSTROMS) | 81.65 |
| REMARK | 3 | DATA CUTOFF | (SIGMA(F)) | NONE |
| REMARK | 3 | COMPLETENESS FOR RANGE | (%) | 99.81 |
| REMARK | 3 | NUMBER OF REFLECTIONS | | 25766 |
| REMARK | 3 | | | |

| REMARK | 3 | FIT TO DATA USED IN REFINEMENT. | |
|---|---|---|---|
| REMARK | 3 | CROSS-VALIDATION METHOD | THROUGHOUT |
| REMARK | 3 | FREE R VALUE TEST SET SELECTION | RANDOM |
| REMARK | 3 | R VALUE (WORKING + TEST SET) | 0.19077 |
| REMARK | 3 | R VALUE (WORKING SET) | 0.18920 |
| REMARK | 3 | FREE R VALUE | 0.22121 |
| REMARK | 3 | FREE R VALUE TEST SET SIZE (%) | 5.0 |
| REMARK | 3 | FREE R VALUE TEST SET COUNT | 1368 |
| REMARK | 3 | | |
| REMARK | 3 | FIT IN THE HIGHEST RESOLUTION BIN. | |
| REMARK | 3 | TOTAL NUMBER OF BINS USED | 20 |
| REMARK | 3 | BIN RESOLUTION RANGE HIGH | 2.030 |
| REMARK | 3 | BIN RESOLUTION RANGE LOW | 2.083 |
| REMARK | 3 | REFLECTION IN BIN (WORKING SET) | 1894 |
| REMARK | 3 | BIN R VALUE (WORKING SET) | 0.289 |
| REMARK | 3 | BIN FREE R VALUE SET COUNT | 113 |
| REMARK | 3 | BIN FREE R VALUE | 0.297 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | 2400 |
| REMARK | 3 | | |

| REMARK | 3 | B VALUES. | | |
|---|---|---|---|---|
| REMARK | 3 | FROM WILSON PLOT | (A**2) | NULL |
| REMARK | 3 | MEAN B VALUE | (OVERALL, A**2) | 27.297 |

| REMARK | 3 | OVERALL ANISOTROPIC B VALUE. | |
|---|---|---|---|
| REMARK | 3 | B11 (A**2) | 0.50 |
| REMARK | 3 | B22 (A**2) | 0.50 |
| REMARK | 3 | B33 (A**2) | −0.74 |
| REMARK | 3 | B12 (A**2) | 0.25 |
| REMARK | 3 | B13 (A**2) | 0.00 |
| REMARK | 3 | B23 (A**2) | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A) | 0.151 |
| REMARK | 3 | ESU BASED ON FREE R VALUE | (A) | 0.140 |
| REMARK | 3 | ESU BASED ON MAXIMUM LIKELIHOOD | (A) | 0.105 |
| REMARK | 3 | ESU FOR B VALUES BASED ON MAXIMUM LIKELIHOOD | (A**2) | 3.960 |
| REMARK | 3 | | | |

| | | | |
|---|---|---|---|
| REMARK | 3 | CORRELATION COEFFICIENTS. | |
| REMARK | 3 | CORRELATION COEFFICIENT FO-FC | 0.959 |
| REMARK | 3 | CORRELATION COEFFICIENT FO-FC FREE | 0.946 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A) | 2334 | 0.011 | 0.021 |
| REMARK | 3 | BOND ANGLES REFINED ATOMS | (DEGREES) | 3174 | 1.105 | 1.959 |
| REMARK | 3 | TORSION ANGLES, PERIOD 1 | (DEGREES) | 273 | 5.228 | 5.000 |
| REMARK | 3 | CHIRAL-CENTER RESTRAINTS | (A**3) | 336 | 0.080 | 0.200 |

| | | | | | | |
|---|---|---|---|---|---|---|
| REMARK | 3 | GENERAL PLANES REFINED ATOMS | (A) | 1800 | 0.004 | 0.020 |
| REMARK | 3 | NON-BONDED CONTACTS REFINED ATOMS | (A) | 1070 | 0.202 | 0.200 |
| REMARK | 3 | H-BOND (X...Y) REFINED ATOMS | (A) | 150 | 0.145 | 0.200 |
| REMARK | 3 | SYMMETRY VDW REFINED ATOMS | (A) | 46 | 0.199 | 0.200 |
| REMARK | 3 | SYMMETRY H-BOND REFINED ATOMS | (A) | 10 | 0.267 | 0.200 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2) | 1365 | 0.799 | 1.500 |
| REMARK | 3 | MAIN-CHAIN ANGLE REFINED ATOMS | (A**2) | 2214 | 1.519 | 2.000 |
| REMARK | 3 | SIDE-CHAIN BOND REFINED ATOMS | (A**2) | 969 | 2.024 | 3.000 |
| REMARK | 3 | SIDE-CHAIN ANGLE REFINED ATOMS | (A**2) | 960 | 3.247 | 4.500 |
| REMARK | 3 | | | | | |

| REMARK | 3 | NCS RESTRAINTS STATISTICS | | | | | |
|---|---|---|---|---|---|---|---|
| REMARK | 3 | NUMBER OF NCS GROUPS NULL | | | | | |
| REMARK | 3 | | | | | | |
| REMARK | 3 | | | | | | |
| REMARK | 3 | TLS DETAILS | | | | | |
| REMARK | 3 | NUMBER OF TLS GROUPS 2 | | | | | |
| REMARK | 3 | | | | | | |
| REMARK | 3 | TLS GROUP 1 | | | | | |
| REMARK | 3 | NUMBER OF COMPONENTS GROUP 1 | | | | | |
| REMARK | 3 | COMPONENTS | C | SSSEQI | TO | C | SSSEQI |
| REMARK | 3 | RESIDUE RANGE | A | 33 | | A | 306 |

| REMARK | 3 | ORIGIN FOR THE GROUP (A) 65.5800 27.1270 −0.6960 | | | |
|---|---|---|---|---|---|
| REMARK | 3 | T TENSOR | | | |
| REMARK | 3 | T11 | 0.1410 T22 | 0.1266 | |
| REMARK | 3 | T33 | 0.0824 T12 | −0.0364 | |
| REMARK | 3 | T13 | −0.0112 T23 | −0.0301 | |
| REMARK | 3 | L TENSOR | | | |
| REMARK | 3 | L11 | 1.3945 L22 | 0.7253 | |
| REMARK | 3 | L33 | 0.8680 L12 | 0.1248 | |
| REMARK | 3 | L13 | −0.3386 L23 | 0.0070 | |
| REMARK | 3 | S TENSOR | | | |
| REMARK | 3 | S11 | −0.0668 S12 | 0.0858 S13 | 0.0787 |
| REMARK | 3 | S21 | −0.0201 S22 | 0.1089 S23 | 0.0287 |
| REMARK | 3 | S31 | 0.0298 S32 | 0.0689 S33 | −0.0421 |
| REMARK | 3 | | | | |

| REMARK | 3 | TLS GROUP 2 | | | | | |
|---|---|---|---|---|---|---|---|
| REMARK | 3 | NUMBER OF COMPONENTS GROUP 1 | | | | | |
| REMARK | 3 | COMPONENTS | C | SSSEQI | TO | C | SSSEQI |
| REMARK | 3 | RESIDUE RANGE | L | 1 | | L | 1 |

| REMARK | 3 | ORIGIN FOR THE GROUP (A) 73.8810 32.6080 1.3720 | | | |
|---|---|---|---|---|---|
| REMARK | 3 | T TENSOR | | | |
| REMARK | 3 | T11 | 0.1174 T22 | 0.1943 | |
| REMARK | 3 | T33 | 0.1391 T12 | −0.1003 | |
| REMARK | 3 | T13 | −0.0486 T23 | −0.0722 | |
| REMARK | 3 | L TENSOR | | | |
| REMARK | 3 | L11 | 14.6629 L22 | 15.7148 | |
| REMARK | 3 | L33 | 8.9109 L12 | −11.1563 | |
| REMARK | 3 | L13 | −1.8290 L23 | −15.1157 | |
| REMARK | 3 | S TENSOR | | | |
| REMARK | 3 | S11 | 0.2483 S12 | 0.1966 S13 | 0.0403 |
| REMARK | 3 | S21 | 0.0302 S22 | 0.1725 S23 | 0.8712 |
| REMARK | 3 | S31 | −0.4688 S32 | 0.8689 S33 | −0.4208 |
| REMARK | 3 | | | | |
| REMARK | 3 | | | | |

| REMARK | 3 | BULK SOLVENT MODELLING. | |
|---|---|---|---|
| REMARK | 3 | METHOD USED BABINET MODEL WITH MASK | |
| REMARK | 3 | PARAMETERS FOR MASK CALCULATION | |
| REMARK | 3 | VDW PROBE RADIUS | 1.40 |
| REMARK | 3 | ION PROBE RADIUS | 0.80 |
| REMARK | 3 | SHRINKAGE RADIUS | 0.80 |
| REMARK | 3 | | |

| | | |
|---|---|---|
| REMARK | 3 | OTHER REFINEMENT REMARKS NULL |
| REMARK | 3 | |

| | |
|---|---|
| CISPEP | 1 GLU A 124 PRO A 125 0.00 |
| CRYST1 | 95.566 95.566 80.862 90.00 90.00 120.00 P 65 |
| SCALE1 | 0.010464 0.006041 0.000000 0.00000 |
| SCALE2 | 0.000000 0.012083 0.000000 0.00000 |
| SCALE3 | 0.000000 0.000000 0.012367 0.00000 |

| | | | | | | | | | | | |
|---|---|---|---|---|---|---|---|---|---|---|---|
| ATOM | 1 | N | PRO | A | 33 | 89.149 | 40.408 | −18.445 | 1.00 | 67.30 | N |
| ATOM | 2 | CA | PRO | A | 33 | 88.476 | 41.476 | −17.647 | 1.00 | 67.14 | C |
| ATOM | 3 | CB | PRO | A | 33 | 86.997 | 41.088 | −17.742 | 1.00 | 67.23 | C |
| ATOM | 4 | CG | PRO | A | 33 | 86.877 | 40.393 | −19.088 | 1.00 | 67.40 | C |
| ATOM | 5 | CD | PRO | A | 33 | 88.243 | 39.825 | −19.451 | 1.00 | 67.35 | C |
| ATOM | 6 | C | PRO | A | 33 | 88.938 | 41.544 | −16.180 | 1.00 | 66.89 | C |
| ATOM | 7 | O | PRO | A | 33 | 89.154 | 42.657 | −15.690 | 1.00 | 67.00 | O |
| ATOM | 8 | N | LEU | A | 34 | 89.091 | 40.388 | −15.519 | 1.00 | 66.32 | N |
| ATOM | 9 | CA | LEU | A | 34 | 89.499 | 40.274 | −14.100 | 1.00 | 65.68 | C |
| ATOM | 10 | CB | LEU | A | 34 | 90.888 | 40.895 | −13.835 | 1.00 | 65.85 | C |
| ATOM | 11 | CG | LEU | A | 34 | 91.302 | 41.230 | −12.390 | 1.00 | 66.30 | C |
| ATOM | 12 | CD1 | LEU | A | 34 | 91.714 | 39.982 | −11.600 | 1.00 | 66.80 | C |
| ATOM | 13 | CD2 | LEU | A | 34 | 92.418 | 42.268 | −12.376 | 1.00 | 67.23 | C |
| ATOM | 14 | C | LEU | A | 34 | 88.454 | 40.795 | −13.100 | 1.00 | 64.94 | C |
| ATOM | 15 | O | LEU | A | 34 | 87.873 | 41.869 | −13.284 | 1.00 | 64.93 | O |
| ATOM | 16 | N | GLU | A | 35 | 88.242 | 40.036 | −12.027 | 1.00 | 63.77 | N |
| ATOM | 17 | CA | GLU | A | 35 | 87.186 | 40.343 | −11.061 | 1.00 | 62.65 | C |
| ATOM | 18 | CB | GLU | A | 35 | 86.798 | 39.087 | −10.270 | 1.00 | 63.13 | C |
| ATOM | 19 | CG | GLU | A | 35 | 87.856 | 38.599 | −9.297 | 1.00 | 65.02 | C |
| ATOM | 20 | CD | GLU | A | 35 | 87.245 | 37.871 | −8.122 | 1.00 | 67.48 | C |
| ATOM | 21 | OE1 | GLU | A | 35 | 87.017 | 38.518 | −7.069 | 1.00 | 68.53 | O |
| ATOM | 22 | OE2 | GLU | A | 35 | 86.987 | 36.654 | −8.260 | 1.00 | 68.44 | O |
| ATOM | 23 | C | GLU | A | 35 | 87.444 | 41.549 | −10.130 | 1.00 | 61.12 | C |
| ATOM | 24 | O | GLU | A | 35 | 86.859 | 41.645 | −9.049 | 1.00 | 61.05 | O |
| ATOM | 25 | N | SER | A | 36 | 88.299 | 42.477 | −10.561 | 1.00 | 59.16 | N |
| ATOM | 26 | CA | SER | A | 36 | 88.360 | 43.797 | −9.923 | 1.00 | 56.76 | C |
| ATOM | 27 | CB | SER | A | 36 | 89.767 | 44.375 | −9.977 | 1.00 | 57.09 | C |
| ATOM | 28 | OG | SER | A | 36 | 90.185 | 44.711 | −8.665 | 1.00 | 57.93 | O |
| ATOM | 29 | C | SER | A | 36 | 87.321 | 44.757 | −10.537 | 1.00 | 54.68 | C |
| ATOM | 30 | O | SER | A | 36 | 87.465 | 45.987 | −10.482 | 1.00 | 54.42 | O |
| ATOM | 31 | N | GLN | A | 37 | 86.278 | 44.159 | −11.121 | 1.00 | 51.69 | N |
| ATOM | 32 | CA | GLN | A | 37 | 85.055 | 44.844 | −11.531 | 1.00 | 48.71 | C |
| ATOM | 33 | CB | GLN | A | 37 | 84.288 | 44.000 | −12.556 | 1.00 | 48.70 | C |
| ATOM | 34 | CG | GLN | A | 37 | 85.032 | 43.705 | −13.853 | 1.00 | 48.89 | C |
| ATOM | 35 | CD | GLN | A | 37 | 84.357 | 42.614 | −14.681 | 1.00 | 48.93 | C |
| ATOM | 36 | OE1 | GLN | A | 37 | 83.235 | 42.790 | −15.159 | 1.00 | 48.57 | O |
| ATOM | 37 | NE2 | GLN | A | 37 | 85.041 | 41.490 | −14.849 | 1.00 | 49.23 | N |
| ATOM | 38 | C | GLN | A | 37 | 84.159 | 45.068 | −10.309 | 1.00 | 46.45 | C |
| ATOM | 39 | O | GLN | A | 37 | 83.061 | 45.621 | −10.425 | 1.00 | 45.80 | O |
| ATOM | 40 | N | TYR | A | 38 | 84.634 | 44.634 | −9.142 | 1.00 | 43.79 | N |
| ATOM | 41 | CA | TYR | A | 38 | 83.815 | 44.599 | −7.935 | 1.00 | 41.47 | C |
| ATOM | 42 | CB | TYR | A | 38 | 83.277 | 43.178 | −7.683 | 1.00 | 40.80 | C |
| ATOM | 43 | CG | TYR | A | 38 | 82.415 | 42.680 | −8.813 | 1.00 | 37.71 | C |
| ATOM | 44 | CD1 | TYR | A | 38 | 81.078 | 43.060 | −8.913 | 1.00 | 36.03 | C |
| ATOM | 45 | CE1 | TYR | A | 38 | 80.283 | 42.622 | −9.970 | 1.00 | 34.69 | C |
| ATOM | 46 | CZ | TYR | A | 38 | 80.834 | 41.799 | −10.940 | 1.00 | 33.35 | C |
| ATOM | 47 | OH | TYR | A | 38 | 80.058 | 41.362 | −11.982 | 1.00 | 33.33 | O |
| ATOM | 48 | CE2 | TYR | A | 38 | 82.156 | 41.409 | −10.861 | 1.00 | 33.59 | C |
| ATOM | 49 | CD2 | TYR | A | 38 | 82.941 | 41.853 | −9.801 | 1.00 | 35.25 | C |
| ATOM | 50 | C | TYR | A | 38 | 84.546 | 45.110 | −6.711 | 1.00 | 40.70 | C |
| ATOM | 51 | O | TYR | A | 38 | 85.729 | 44.835 | −6.522 | 1.00 | 40.55 | O |
| ATOM | 52 | N | GLN | A | 39 | 83.820 | 45.882 | −5.907 | 1.00 | 39.47 | N |
| ATOM | 53 | CA | GLN | A | 39 | 84.267 | 46.331 | −4.602 | 1.00 | 38.56 | C |
| ATOM | 54 | CB | GLN | A | 39 | 83.781 | 47.755 | −4.350 | 1.00 | 39.00 | C |
| ATOM | 55 | CG | GLN | A | 39 | 84.391 | 48.448 | −3.148 | 1.00 | 41.12 | C |
| ATOM | 56 | CD | GLN | A | 39 | 83.988 | 49.920 | −3.066 | 1.00 | 44.96 | C |
| ATOM | 57 | OE1 | GLN | A | 39 | 84.489 | 50.753 | −3.833 | 1.00 | 45.76 | O |
| ATOM | 58 | NE2 | GLN | A | 39 | 83.081 | 50.241 | −2.139 | 1.00 | 46.18 | N |
| ATOM | 59 | C | GLN | A | 39 | 83.673 | 45.376 | −3.567 | 1.00 | 37.29 | C |
| ATOM | 60 | O | GLN | A | 39 | 82.451 | 45.224 | −3.473 | 1.00 | 36.66 | O |
| ATOM | 61 | N | VAL | A | 40 | 84.546 | 44.738 | −2.797 | 1.00 | 35.79 | N |
| ATOM | 62 | CA | VAL | A | 40 | 84.124 | 43.757 | −1.808 | 1.00 | 34.34 | C |
| ATOM | 63 | CB | VAL | A | 40 | 85.190 | 42.667 | −1.580 | 1.00 | 34.42 | C |
| ATOM | 64 | CG1 | VAL | A | 40 | 84.573 | 41.470 | −0.871 | 1.00 | 34.56 | C |
| ATOM | 65 | CG2 | VAL | A | 40 | 85.822 | 42.238 | −2.908 | 1.00 | 34.84 | C |
| ATOM | 66 | C | VAL | A | 40 | 83.794 | 44.435 | −0.487 | 1.00 | 33.17 | C |
| ATOM | 67 | O | VAL | A | 40 | 84.544 | 45.296 | −0.021 | 1.00 | 32.88 | O |
| ATOM | 68 | N | GLY | A | 41 | 82.659 | 44.047 | 0.094 | 1.00 | 31.35 | N |
| ATOM | 69 | CA | GLY | A | 41 | 82.253 | 44.508 | 1.407 | 1.00 | 29.70 | C |
| ATOM | 70 | C | GLY | A | 41 | 82.304 | 43.395 | 2.438 | 1.00 | 28.50 | C |
| ATOM | 71 | O | GLY | A | 41 | 83.121 | 42.480 | 2.315 | 1.00 | 28.62 | O |
| ATOM | 72 | N | PRO | A | 42 | 81.435 | 43.470 | 3.446 | 1.00 | 27.60 | N |
| ATOM | 73 | CA | PRO | A | 42 | 81.406 | 42.494 | 4.543 | 1.00 | 27.00 | C |
| ATOM | 74 | CB | PRO | A | 42 | 80.355 | 43.073 | 5.501 | 1.00 | 27.10 | C |
| ATOM | 75 | CG | PRO | A | 42 | 80.182 | 44.506 | 5.089 | 1.00 | 27.33 | C |
| ATOM | 76 | CD | PRO | A | 42 | 80.416 | 44.521 | 3.618 | 1.00 | 27.63 | C |
| ATOM | 77 | C | PRO | A | 42 | 80.958 | 41.091 | 4.127 | 1.00 | 26.95 | C |
| ATOM | 78 | O | PRO | A | 42 | 80.212 | 40.921 | 3.149 | 1.00 | 26.22 | O |
| ATOM | 79 | N | LEU | A | 43 | 81.421 | 40.114 | 4.905 | 1.00 | 26.40 | N |
| ATOM | 80 | CA | LEU | A | 43 | 81.016 | 38.726 | 4.827 | 1.00 | 26.26 | C |
| ATOM | 81 | CB | LEU | A | 43 | 81.928 | 37.888 | 5.737 | 1.00 | 26.16 | C |
| ATOM | 82 | CG | LEU | A | 43 | 81.741 | 36.367 | 5.836 | 1.00 | 26.71 | C |
| ATOM | 83 | CD1 | LEU | A | 43 | 81.971 | 35.666 | 4.486 | 1.00 | 25.17 | C |
| ATOM | 84 | CD2 | LEU | A | 43 | 82.656 | 35.790 | 6.911 | 1.00 | 25.63 | C |
| ATOM | 85 | C | LEU | A | 43 | 79.573 | 38.592 | 5.292 | 1.00 | 26.23 | C |
| ATOM | 86 | O | LEU | A | 43 | 79.234 | 39.010 | 6.409 | 1.00 | 25.53 | O |
| ATOM | 87 | N | LEU | A | 44 | 78.737 | 37.998 | 4.439 | 1.00 | 25.89 | N |
| ATOM | 88 | CA | LEU | A | 44 | 77.321 | 37.786 | 4.746 | 1.00 | 26.23 | C |
| ATOM | 89 | CB | LEU | A | 44 | 76.460 | 37.994 | 3.500 | 1.00 | 25.73 | C |
| ATOM | 90 | CG | LEU | A | 44 | 76.500 | 39.383 | 2.881 | 1.00 | 25.94 | C |
| ATOM | 91 | CD1 | LEU | A | 44 | 75.804 | 39.381 | 1.516 | 1.00 | 24.87 | C |
| ATOM | 92 | CD2 | LEU | A | 44 | 75.881 | 40.399 | 3.846 | 1.00 | 26.24 | C |
| ATOM | 93 | C | LEU | A | 44 | 77.027 | 36.416 | 5.345 | 1.00 | 26.74 | C |
| ATOM | 94 | O | LEU | A | 44 | 76.107 | 36.274 | 6.148 | 1.00 | 26.63 | O |
| ATOM | 95 | N | GLY | A | 45 | 77.798 | 35.409 | 4.946 | 1.00 | 27.42 | N |
| ATOM | 96 | CA | GLY | A | 45 | 77.595 | 34.056 | 5.434 | 1.00 | 28.81 | C |
| ATOM | 97 | C | GLY | A | 45 | 78.642 | 33.077 | 4.932 | 1.00 | 29.90 | C |
| ATOM | 98 | O | GLY | A | 45 | 79.209 | 33.254 | 3.854 | 1.00 | 29.36 | O |
| ATOM | 99 | N | SER | A | 46 | 78.908 | 32.061 | 5.745 | 1.00 | 31.14 | N |
| ATOM | 100 | CA | SER | A | 46 | 79.794 | 30.964 | 5.385 | 1.00 | 33.14 | C |
| ATOM | 101 | CB | SER | A | 46 | 81.242 | 31.282 | 5.786 | 1.00 | 33.24 | C |
| ATOM | 102 | OG | SER | A | 46 | 81.336 | 31.607 | 7.161 | 1.00 | 31.40 | O |
| ATOM | 103 | C | SER | A | 46 | 79.263 | 29.723 | 6.104 | 1.00 | 34.64 | C |
| ATOM | 104 | O | SER | A | 46 | 78.131 | 29.727 | 6.594 | 1.00 | 35.16 | O |
| ATOM | 105 | N | GLY | A | 47 | 80.033 | 28.645 | 6.168 | 1.00 | 36.14 | N |
| ATOM | 106 | CA | GLY | A | 47 | 79.552 | 27.513 | 6.960 | 1.00 | 37.77 | C |
| ATOM | 107 | C | GLY | A | 47 | 78.827 | 26.410 | 6.202 | 1.00 | 38.06 | C |
| ATOM | 108 | O | GLY | A | 47 | 78.803 | 25.261 | 6.665 | 1.00 | 38.85 | O |
| ATOM | 109 | N | GLY | A | 48 | 78.228 | 26.756 | 5.058 | 1.00 | 38.13 | N |
| ATOM | 110 | CA | GLY | A | 48 | 77.816 | 25.765 | 4.072 | 1.00 | 37.47 | C |
| ATOM | 111 | C | GLY | A | 48 | 79.007 | 25.489 | 3.163 | 1.00 | 37.20 | C |
| ATOM | 112 | O | GLY | A | 48 | 80.154 | 25.459 | 3.631 | 1.00 | 37.27 | O |
| ATOM | 113 | N | PHE | A | 49 | 78.757 | 25.324 | 1.865 | 1.00 | 36.60 | N |
| ATOM | 114 | CA | PHE | A | 49 | 79.845 | 25.099 | 0.905 | 1.00 | 36.12 | C |
| ATOM | 115 | CB | PHE | A | 49 | 79.322 | 24.532 | −0.421 | 1.00 | 36.73 | C |
| ATOM | 116 | CG | PHE | A | 49 | 78.733 | 23.153 | −0.310 | 1.00 | 39.10 | C |
| ATOM | 117 | CD1 | PHE | A | 49 | 77.363 | 22.960 | −0.454 | 1.00 | 40.06 | C |
| ATOM | 118 | CE1 | PHE | A | 49 | 76.806 | 21.681 | −0.357 | 1.00 | 42.14 | C |
| ATOM | 119 | CZ | PHE | A | 49 | 77.624 | 20.575 | −0.105 | 1.00 | 43.04 | C |
| ATOM | 120 | CE2 | PHE | A | 49 | 79.003 | 20.755 | 0.045 | 1.00 | 43.48 | C |
| ATOM | 121 | CD2 | PHE | A | 49 | 79.550 | 22.043 | −0.061 | 1.00 | 42.11 | C |
| ATOM | 122 | C | PHE | A | 49 | 80.702 | 26.339 | 0.614 | 1.00 | 34.83 | C |
| ATOM | 123 | O | PHE | A | 49 | 81.884 | 26.195 | 0.286 | 1.00 | 35.14 | O |
| ATOM | 124 | N | GLY | A | 50 | 80.109 | 27.536 | 0.717 | 1.00 | 32.72 | N |
| ATOM | 125 | CA | GLY | A | 50 | 80.770 | 28.772 | 0.303 | 1.00 | 30.18 | C |
| ATOM | 126 | C | GLY | A | 50 | 80.831 | 29.895 | 1.335 | 1.00 | 28.53 | C |
| ATOM | 127 | O | GLY | A | 50 | 80.161 | 29.832 | 2.367 | 1.00 | 28.21 | O |
| ATOM | 128 | N | SER | A | 51 | 81.676 | 30.895 | 1.061 | 1.00 | 26.40 | N |
| ATOM | 129 | CA | SER | A | 51 | 81.722 | 32.156 | 1.803 | 1.00 | 23.83 | C |
| ATOM | 130 | CB | SER | A | 51 | 83.157 | 32.509 | 2.190 | 1.00 | 24.19 | C |
| ATOM | 131 | OG | SER | A | 51 | 83.773 | 31.474 | 2.937 | 1.00 | 23.82 | O |
| ATOM | 132 | C | SER | A | 51 | 81.167 | 33.245 | 0.888 | 1.00 | 22.65 | C |
| ATOM | 133 | O | SER | A | 51 | 81.640 | 33.423 | −0.242 | 1.00 | 21.83 | O |
| ATOM | 134 | N | VAL | A | 52 | 80.150 | 33.948 | 1.369 | 1.00 | 20.93 | N |
| ATOM | 135 | CA | VAL | A | 52 | 79.427 | 34.917 | 0.568 | 1.00 | 20.09 | C |
| ATOM | 136 | CB | VAL | A | 52 | 77.917 | 34.574 | 0.518 | 1.00 | 19.63 | C |
| ATOM | 137 | CG1 | VAL | A | 52 | 77.182 | 35.536 | −0.391 | 1.00 | 19.68 | C |
| ATOM | 138 | CG2 | VAL | A | 52 | 77.705 | 33.133 | 0.035 | 1.00 | 19.27 | C |
| ATOM | 139 | C | VAL | A | 52 | 79.629 | 36.330 | 1.119 | 1.00 | 20.59 | C |
| ATOM | 140 | O | VAL | A | 52 | 79.406 | 36.576 | 2.309 | 1.00 | 19.74 | O |
| ATOM | 141 | N | TYR | A | 53 | 80.053 | 37.241 | 0.250 | 1.00 | 20.61 | N |
| ATOM | 142 | CA | TYR | A | 53 | 80.316 | 38.624 | 0.640 | 1.00 | 21.62 | C |
| ATOM | 143 | CB | TYR | A | 53 | 81.737 | 39.034 | 0.247 | 1.00 | 21.19 | C |
| ATOM | 144 | CG | TYR | A | 53 | 82.842 | 38.256 | 0.922 | 1.00 | 22.16 | C |
| ATOM | 145 | CD1 | TYR | A | 53 | 83.201 | 36.980 | 0.470 | 1.00 | 21.94 | C |
| ATOM | 146 | CE1 | TYR | A | 53 | 84.225 | 36.265 | 1.078 | 1.00 | 22.80 | C |
| ATOM | 147 | CZ | TYR | A | 53 | 84.921 | 36.830 | 2.146 | 1.00 | 23.82 | C |
| ATOM | 148 | OH | TYR | A | 53 | 85.937 | 36.114 | 2.736 | 1.00 | 23.97 | O |
| ATOM | 149 | CE2 | TYR | A | 53 | 84.597 | 38.099 | 2.614 | 1.00 | 23.07 | C |
| ATOM | 150 | CD2 | TYR | A | 53 | 83.559 | 38.810 | 1.994 | 1.00 | 23.01 | C |
| ATOM | 151 | C | TYR | A | 53 | 79.354 | 39.597 | −0.024 | 1.00 | 22.34 | C |
| ATOM | 152 | O | TYR | A | 53 | 78.888 | 39.373 | −1.153 | 1.00 | 22.35 | O |
| ATOM | 153 | N | SER | A | 54 | 79.066 | 40.681 | 0.680 | 1.00 | 23.48 | N |
| ATOM | 154 | CA | SER | A | 54 | 78.404 | 41.822 | 0.074 | 1.00 | 24.82 | C |
| ATOM | 155 | CB | SER | A | 54 | 77.980 | 42.840 | 1.140 | 1.00 | 25.09 | C |
| ATOM | 156 | OG | SER | A | 54 | 77.307 | 43.939 | 0.545 | 1.00 | 25.39 | O |
| ATOM | 157 | C | SER | A | 54 | 79.384 | 42.461 | −0.889 | 1.00 | 25.68 | C |
| ATOM | 158 | O | SER | A | 54 | 80.586 | 42.513 | −0.616 | 1.00 | 25.83 | O |
| ATOM | 159 | N | GLY | A | 55 | 78.878 | 42.932 | −2.023 | 1.00 | 26.75 | N |
| ATOM | 160 | CA | GLY | A | 55 | 79.720 | 43.609 | −2.991 | 1.00 | 27.76 | C |
| ATOM | 161 | C | GLY | A | 55 | 78.986 | 44.633 | −3.827 | 1.00 | 28.87 | C |
| ATOM | 162 | O | GLY | A | 55 | 77.762 | 44.772 | −3.737 | 1.00 | 28.62 | O |
| ATOM | 163 | N | ILE | A | 56 | 79.750 | 45.358 | −4.641 | 1.00 | 30.03 | N |
| ATOM | 164 | CA | ILE | A | 56 | 79.200 | 46.346 | −5.557 | 1.00 | 31.52 | C |
| ATOM | 165 | CB | ILE | A | 56 | 79.291 | 47.773 | −4.953 | 1.00 | 31.73 | C |
| ATOM | 166 | CG1 | ILE | A | 56 | 78.306 | 47.955 | −3.790 | 1.00 | 32.00 | C |
| ATOM | 167 | CD1 | ILE | A | 56 | 78.762 | 48.992 | −2.750 | 1.00 | 34.29 | C |
| ATOM | 168 | CG2 | ILE | A | 56 | 79.038 | 48.830 | −6.014 | 1.00 | 32.25 | C |
| ATOM | 169 | C | ILE | A | 56 | 79.927 | 46.274 | −6.901 | 1.00 | 32.27 | C |
| ATOM | 170 | O | ILE | A | 56 | 81.153 | 46.245 | −6.956 | 1.00 | 32.20 | O |
| ATOM | 171 | N | ARG | A | 57 | 79.147 | 46.225 | −7.976 | 1.00 | 33.45 | N |
| ATOM | 172 | CA | ARG | A | 57 | 79.664 | 46.308 | −9.332 | 1.00 | 34.77 | C |
| ATOM | 173 | CB | ARG | A | 57 | 78.574 | 45.902 | −10.319 | 1.00 | 34.53 | C |
| ATOM | 174 | CG | ARG | A | 57 | 79.075 | 45.541 | −11.692 | 1.00 | 35.85 | C |
| ATOM | 175 | CD | ARG | A | 57 | 78.037 | 45.746 | −12.766 | 1.00 | 37.31 | C |
| ATOM | 176 | NE | ARG | A | 57 | 77.459 | 44.488 | −13.210 | 1.00 | 38.72 | N |
| ATOM | 177 | CZ | ARG | A | 57 | 76.191 | 44.334 | −13.580 | 1.00 | 39.18 | C |
| ATOM | 178 | NH1 | ARG | A | 57 | 75.347 | 45.360 | −13.561 | 1.00 | 38.58 | N |
| ATOM | 179 | NH2 | ARG | A | 57 | 75.764 | 43.143 | −13.967 | 1.00 | 40.00 | N |
| ATOM | 180 | C | ARG | A | 57 | 80.134 | 47.740 | −9.604 | 1.00 | 35.42 | C |
| ATOM | 181 | O | ARG | A | 57 | 79.329 | 48.667 | −9.609 | 1.00 | 35.07 | O |
| ATOM | 182 | N | VAL | A | 58 | 81.438 | 47.901 | −9.822 | 1.00 | 36.96 | N |
| ATOM | 183 | CA | VAL | A | 58 | 82.069 | 49.221 | −9.962 | 1.00 | 38.44 | C |
| ATOM | 184 | CB | VAL | A | 58 | 83.626 | 49.118 | −10.083 | 1.00 | 38.53 | C |
| ATOM | 185 | CG1 | VAL | A | 58 | 84.270 | 50.494 | −10.251 | 1.00 | 38.65 | C |
| ATOM | 186 | CG2 | VAL | A | 58 | 84.226 | 48.422 | −8.863 | 1.00 | 38.97 | C |
| ATOM | 187 | C | VAL | A | 58 | 81.472 | 50.033 | −11.125 | 1.00 | 39.26 | C |
| ATOM | 188 | O | VAL | A | 58 | 81.243 | 51.238 | −10.989 | 1.00 | 39.41 | O |
| ATOM | 189 | N | SER | A | 59 | 81.194 | 49.357 | −12.239 | 1.00 | 40.32 | N |
| ATOM | 190 | CA | SER | A | 59 | 80.704 | 50.007 | −13.459 | 1.00 | 41.50 | C |
| ATOM | 191 | CB | SER | A | 59 | 80.627 | 49.012 | −14.625 | 1.00 | 41.60 | C |
| ATOM | 192 | OG | SER | A | 59 | 80.059 | 47.777 | −14.225 | 1.00 | 42.83 | O |
| ATOM | 193 | C | SER | A | 59 | 79.380 | 50.778 | −13.310 | 1.00 | 41.87 | C |
| ATOM | 194 | O | SER | A | 59 | 79.205 | 51.830 | −13.933 | 1.00 | 42.28 | O |
| ATOM | 195 | N | ASP | A | 60 | 78.463 | 50.269 | −12.488 | 1.00 | 42.04 | N |
| ATOM | 196 | CA | ASP | A | 60 | 77.147 | 50.897 | −12.330 | 1.00 | 41.97 | C |
| ATOM | 197 | CB | ASP | A | 60 | 76.118 | 50.187 | −13.223 | 1.00 | 42.37 | C |
| ATOM | 198 | CG | ASP | A | 60 | 75.881 | 48.739 | −12.810 | 1.00 | 43.55 | C |
| ATOM | 199 | OD1 | ASP | A | 60 | 76.449 | 48.309 | −11.781 | 1.00 | 44.32 | O |
| ATOM | 200 | OD2 | ASP | A | 60 | 75.142 | 47.959 | −13.451 | 1.00 | 43.86 | O |
| ATOM | 201 | C | ASP | A | 60 | 76.631 | 50.996 | −10.878 | 1.00 | 41.41 | C |
| ATOM | 202 | O | ASP | A | 60 | 75.479 | 51.372 | −10.657 | 1.00 | 41.59 | O |
| ATOM | 203 | N | ASN | A | 61 | 77.484 | 50.667 | −9.905 | 1.00 | 40.49 | N |
| ATOM | 204 | CA | ASN | A | 61 | 77.122 | 50.639 | −8.475 | 1.00 | 39.60 | C |
| ATOM | 205 | CB | ASN | A | 61 | 76.722 | 52.026 | −7.961 | 1.00 | 39.96 | C |
| ATOM | 206 | CG | ASN | A | 61 | 77.888 | 52.981 | −7.902 | 1.00 | 41.52 | C |
| ATOM | 207 | OD1 | ASN | A | 61 | 78.785 | 52.837 | −7.065 | 1.00 | 42.83 | O |
| ATOM | 208 | ND2 | ASN | A | 61 | 77.883 | 53.971 | −8.792 | 1.00 | 42.64 | N |
| ATOM | 209 | C | ASN | A | 61 | 76.058 | 49.615 | −8.056 | 1.00 | 38.30 | C |
| ATOM | 210 | O | ASN | A | 61 | 75.557 | 49.667 | −6.930 | 1.00 | 38.56 | O |
| ATOM | 211 | N | LEU | A | 62 | 75.724 | 48.685 | −8.947 | 1.00 | 36.52 | N |
| ATOM | 212 | CA | LEU | A | 62 | 74.768 | 47.623 | −8.623 | 1.00 | 34.93 | C |
| ATOM | 213 | CB | LEU | A | 62 | 74.519 | 46.720 | −9.832 | 1.00 | 35.10 | C |
| ATOM | 214 | CG | LEU | A | 62 | 73.421 | 45.662 | −9.712 | 1.00 | 35.38 | C |
| ATOM | 215 | CD1 | LEU | A | 62 | 72.047 | 46.269 | −9.961 | 1.00 | 36.89 | C |
| ATOM | 216 | CD2 | LEU | A | 62 | 73.679 | 44.527 | −10.677 | 1.00 | 35.30 | C |
| ATOM | 217 | C | LEU | A | 62 | 75.222 | 46.778 | −7.425 | 1.00 | 33.33 | C |
| ATOM | 218 | O | LEU | A | 62 | 76.351 | 46.288 | −7.404 | 1.00 | 32.86 | O |
| ATOM | 219 | N | PRO | A | 63 | 74.340 | 46.624 | −6.436 | 1.00 | 32.03 | N |
| ATOM | 220 | CA | PRO | A | 63 | 74.576 | 45.708 | −5.312 | 1.00 | 30.66 | C |
| ATOM | 221 | CB | PRO | A | 63 | 73.328 | 45.893 | −4.440 | 1.00 | 30.79 | C |
| ATOM | 222 | CG | PRO | A | 63 | 72.770 | 47.219 | −4.848 | 1.00 | 32.09 | C |
| ATOM | 223 | CD | PRO | A | 63 | 73.039 | 47.311 | −6.319 | 1.00 | 32.13 | C |
| ATOM | 224 | C | PRO | A | 63 | 74.657 | 44.263 | −5.804 | 1.00 | 28.94 | C |
| ATOM | 225 | O | PRO | A | 63 | 73.788 | 43.821 | −6.570 | 1.00 | 28.84 | O |
| ATOM | 226 | N | VAL | A | 64 | 75.705 | 43.554 | −5.393 | 1.00 | 26.63 | N |
| ATOM | 227 | CA | VAL | A | 64 | 75.862 | 42.135 | −5.723 | 1.00 | 24.40 | C |
| ATOM | 228 | CB | VAL | A | 64 | 76.903 | 41.905 | −6.870 | 1.00 | 24.56 | C |
| ATOM | 229 | CG1 | VAL | A | 64 | 76.430 | 42.528 | −8.195 | 1.00 | 23.44 | C |
| ATOM | 230 | CG2 | VAL | A | 64 | 78.292 | 42.436 | −6.471 | 1.00 | 23.53 | C |
| ATOM | 231 | C | VAL | A | 64 | 76.295 | 41.339 | −4.488 | 1.00 | 23.30 | C |
| ATOM | 232 | O | VAL | A | 64 | 76.650 | 41.922 | −3.451 | 1.00 | 22.79 | O |
| ATOM | 233 | N | ALA | A | 65 | 76.259 | 40.014 | −4.609 | 1.00 | 21.63 | N |
| ATOM | 234 | CA | ALA | A | 65 | 76.828 | 39.124 | −3.608 | 1.00 | 20.83 | C |
| ATOM | 235 | CB | ALA | A | 65 | 75.761 | 38.231 | −2.984 | 1.00 | 20.55 | C |
| ATOM | 236 | C | ALA | A | 65 | 77.892 | 38.290 | −4.281 | 1.00 | 20.04 | C |
| ATOM | 237 | O | ALA | A | 65 | 77.704 | 37.828 | −5.408 | 1.00 | 21.14 | O |
| ATOM | 238 | N | ILE | A | 66 | 79.015 | 38.111 | −3.600 | 1.00 | 18.86 | N |
| ATOM | 239 | CA | ILE | A | 66 | 80.165 | 37.439 | −4.186 | 1.00 | 17.57 | C |
| ATOM | 240 | CB | ILE | A | 66 | 81.422 | 38.346 | −4.117 | 1.00 | 17.68 | C |
| ATOM | 241 | CG1 | ILE | A | 66 | 81.148 | 39.723 | −4.747 | 1.00 | 18.56 | C |
| ATOM | 242 | CD1 | ILE | A | 66 | 82.220 | 40.772 | −4.424 | 1.00 | 19.55 | C |
| ATOM | 243 | CG2 | ILE | A | 66 | 82.602 | 37.668 | −4.775 | 1.00 | 16.32 | C |
| ATOM | 244 | C | ILE | A | 66 | 80.402 | 36.161 | −3.408 | 1.00 | 16.96 | C |
| ATOM | 245 | O | ILE | A | 66 | 80.775 | 36.206 | −2.227 | 1.00 | 16.17 | O |
| ATOM | 246 | N | LYS | A | 67 | 80.179 | 35.031 | −4.077 | 1.00 | 16.24 | N |
| ATOM | 247 | CA | LYS | A | 67 | 80.255 | 33.718 | −3.444 | 1.00 | 15.79 | C |
| ATOM | 248 | CB | LYS | A | 67 | 78.975 | 32.905 | −3.707 | 1.00 | 15.38 | C |
| ATOM | 249 | CG | LYS | A | 67 | 79.010 | 31.493 | −3.119 | 1.00 | 14.88 | C |
| ATOM | 250 | CD | LYS | A | 67 | 77.664 | 30.772 | −3.303 | 1.00 | 17.48 | C |
| ATOM | 251 | CE | LYS | A | 67 | 77.585 | 29.486 | −2.479 | 1.00 | 18.05 | C |
| ATOM | 252 | NZ | LYS | A | 67 | 76.184 | 28.951 | −2.470 | 1.00 | 18.14 | N |
| ATOM | 253 | C | LYS | A | 67 | 81.478 | 32.943 | −3.915 | 1.00 | 16.15 | C |
| ATOM | 254 | O | LYS | A | 67 | 81.667 | 32.705 | −5.122 | 1.00 | 15.33 | O |
| ATOM | 255 | N | HIS | A | 68 | 82.293 | 32.534 | −2.951 | 1.00 | 16.82 | N |
| ATOM | 256 | CA | HIS | A | 68 | 83.519 | 31.792 | −3.235 | 1.00 | 17.55 | C |
| ATOM | 257 | CB | HIS | A | 68 | 84.683 | 32.368 | −2.435 | 1.00 | 17.11 | C |
| ATOM | 258 | CG | HIS | A | 68 | 85.043 | 33.764 | −2.818 | 1.00 | 17.46 | C |
| ATOM | 259 | ND1 | HIS | A | 68 | 84.358 | 34.860 | −2.348 | 1.00 | 18.28 | N |
| ATOM | 260 | CE1 | HIS | A | 68 | 84.897 | 35.958 | −2.844 | 1.00 | 17.55 | C |
| ATOM | 261 | NE2 | HIS | A | 68 | 85.909 | 35.614 | −3.617 | 1.00 | 17.90 | N |
| ATOM | 262 | CD2 | HIS | A | 68 | 86.019 | 34.245 | −3.622 | 1.00 | 17.05 | C |
| ATOM | 263 | C | HIS | A | 68 | 83.319 | 30.353 | −2.829 | 1.00 | 18.47 | C |
| ATOM | 264 | O | HIS | A | 68 | 82.899 | 30.085 | −1.707 | 1.00 | 17.74 | O |
| ATOM | 265 | N | VAL | A | 69 | 83.628 | 29.434 | −3.735 | 1.00 | 19.87 | N |
| ATOM | 266 | CA | VAL | A | 69 | 83.538 | 28.016 | −3.441 | 1.00 | 21.97 | C |
| ATOM | 267 | CB | VAL | A | 69 | 82.386 | 27.316 | −4.229 | 1.00 | 22.09 | C |
| ATOM | 268 | CG1 | VAL | A | 69 | 82.270 | 25.863 | −3.809 | 1.00 | 22.01 | C |
| ATOM | 269 | CG2 | VAL | A | 69 | 81.049 | 28.011 | −3.992 | 1.00 | 22.34 | C |
| ATOM | 270 | C | VAL | A | 69 | 84.870 | 27.345 | −3.768 | 1.00 | 23.59 | C |
| ATOM | 271 | O | VAL | A | 69 | 85.331 | 27.388 | −4.903 | 1.00 | 23.28 | O |
| ATOM | 272 | N | GLU | A | 70 | 85.474 | 26.719 | −2.766 | 1.00 | 26.14 | N |
| ATOM | 273 | CA | GLU | A | 70 | 86.719 | 25.981 | −2.948 | 1.00 | 29.32 | C |
| ATOM | 274 | CB | GLU | A | 70 | 87.280 | 25.543 | −1.599 | 1.00 | 29.52 | C |
| ATOM | 275 | CG | GLU | A | 70 | 88.286 | 26.512 | −1.001 | 1.00 | 32.13 | C |
| ATOM | 276 | CD | GLU | A | 70 | 88.827 | 26.043 | 0.342 | 1.00 | 34.86 | C |
| ATOM | 277 | OE1 | GLU | A | 70 | 89.185 | 24.847 | 0.448 | 1.00 | 34.79 | O |
| ATOM | 278 | OE2 | GLU | A | 70 | 88.899 | 26.871 | 1.288 | 1.00 | 35.33 | O |
| ATOM | 279 | C | GLU | A | 70 | 86.486 | 24.760 | −3.834 | 1.00 | 31.18 | C |
| ATOM | 280 | O | GLU | A | 70 | 85.485 | 24.044 | −3.674 | 1.00 | 30.70 | O |
| ATOM | 281 | N | LYS | A | 71 | 87.402 | 24.540 | −4.774 | 1.00 | 33.73 | N |
| ATOM | 282 | CA | LYS | A | 71 | 87.292 | 23.422 | −5.718 | 1.00 | 36.85 | C |
| ATOM | 283 | CB | LYS | A | 71 | 88.426 | 23.459 | −6.734 | 1.00 | 36.40 | C |
| ATOM | 284 | CG | LYS | A | 71 | 88.228 | 24.487 | −7.822 | 1.00 | 35.73 | C |
| ATOM | 285 | CD | LYS | A | 71 | 89.373 | 24.457 | −8.814 | 1.00 | 35.72 | C |
| ATOM | 286 | CE | LYS | A | 71 | 89.168 | 25.490 | −9.898 | 1.00 | 35.77 | C |
| ATOM | 287 | NZ | LYS | A | 71 | 90.289 | 25.535 | −10.874 | 1.00 | 35.56 | N |
| ATOM | 288 | C | LYS | A | 71 | 87.206 | 22.046 | −5.047 | 1.00 | 39.35 | C |
| ATOM | 289 | O | LYS | A | 71 | 86.492 | 21.169 | −5.536 | 1.00 | 39.62 | O |
| ATOM | 290 | N | ASP | A | 72 | 87.904 | 21.872 | −3.922 | 1.00 | 42.63 | N |
| ATOM | 291 | CA | ASP | A | 72 | 87.873 | 20.615 | −3.161 | 1.00 | 46.03 | C |
| ATOM | 292 | CB | ASP | A | 72 | 89.021 | 20.560 | −2.145 | 1.00 | 46.33 | C |
| ATOM | 293 | CG | ASP | A | 72 | 90.396 | 20.511 | −2.811 | 1.00 | 48.21 | C |
| ATOM | 294 | OD1 | ASP | A | 72 | 90.519 | 19.935 | −3.918 | 1.00 | 49.79 | O |
| ATOM | 295 | OD2 | ASP | A | 72 | 91.418 | 21.025 | −2.300 | 1.00 | 50.39 | O |
| ATOM | 296 | C | ASP | A | 72 | 86.539 | 20.371 | −2.452 | 1.00 | 47.89 | C |
| ATOM | 297 | O | ASP | A | 72 | 86.138 | 19.221 | −2.253 | 1.00 | 48.61 | O |
| ATOM | 298 | N | ARG | A | 73 | 85.861 | 21.457 | −2.085 | 1.00 | 50.08 | N |
| ATOM | 299 | CA | ARG | A | 73 | 84.592 | 21.405 | −1.352 | 1.00 | 52.11 | C |
| ATOM | 300 | CB | ARG | A | 73 | 84.430 | 22.662 | −0.486 | 1.00 | 52.39 | C |
| ATOM | 301 | CG | ARG | A | 73 | 85.333 | 22.711 | 0.739 | 1.00 | 54.59 | C |
| ATOM | 302 | CD | ARG | A | 73 | 84.894 | 23.708 | 1.827 | 1.00 | 58.87 | C |
| ATOM | 303 | NE | ARG | A | 73 | 83.451 | 23.686 | 2.113 | 1.00 | 62.15 | N |
| ATOM | 304 | CZ | ARG | A | 73 | 82.792 | 22.682 | 2.708 | 1.00 | 63.76 | C |
| ATOM | 305 | NH1 | ARG | A | 73 | 83.427 | 21.579 | 3.094 | 1.00 | 64.18 | N |
| ATOM | 306 | NH2 | ARG | A | 73 | 81.484 | 22.779 | 2.917 | 1.00 | 63.67 | N |
| ATOM | 307 | C | ARG | A | 73 | 83.376 | 21.251 | −2.272 | 1.00 | 52.88 | C |
| ATOM | 308 | O | ARG | A | 73 | 82.246 | 21.100 | −1.793 | 1.00 | 52.85 | O |
| ATOM | 309 | N | ILE | A | 74 | 83.613 | 21.307 | −3.585 | 1.00 | 54.14 | N |
| ATOM | 310 | CA | ILE | A | 74 | 82.553 | 21.153 | −4.583 | 1.00 | 55.27 | C |
| ATOM | 311 | CB | ILE | A | 74 | 83.013 | 21.668 | −5.983 | 1.00 | 55.13 | C |
| ATOM | 312 | CG1 | ILE | A | 74 | 83.104 | 23.193 | −5.985 | 1.00 | 54.96 | C |
| ATOM | 313 | CD1 | ILE | A | 74 | 83.829 | 23.776 | −7.180 | 1.00 | 55.18 | C |
| ATOM | 314 | CG2 | ILE | A | 74 | 82.053 | 21.205 | −7.084 | 1.00 | 55.44 | C |
| ATOM | 315 | C | ILE | A | 74 | 82.107 | 19.691 | −4.638 | 1.00 | 56.17 | C |
| ATOM | 316 | O | ILE | A | 74 | 82.902 | 18.798 | −4.973 | 1.00 | 56.10 | O |
| ATOM | 317 | N | SER | A | 75 | 80.836 | 19.459 | −4.298 | 1.00 | 57.17 | N |
| ATOM | 318 | CA | SER | A | 75 | 80.287 | 18.101 | −4.234 | 1.00 | 58.19 | C |
| ATOM | 319 | CB | SER | A | 75 | 78.943 | 18.064 | −3.485 | 1.00 | 58.21 | C |
| ATOM | 320 | OG | SER | A | 75 | 78.112 | 19.161 | −3.831 | 1.00 | 58.80 | O |
| ATOM | 321 | C | SER | A | 75 | 80.178 | 17.482 | −5.629 | 1.00 | 58.59 | C |
| ATOM | 322 | O | SER | A | 75 | 80.890 | 16.520 | −5.939 | 1.00 | 58.89 | O |
| ATOM | 323 | N | ASP | A | 76 | 79.313 | 18.055 | −6.469 | 1.00 | 58.93 | N |
| ATOM | 324 | CA | ASP | A | 76 | 79.125 | 17.581 | −7.840 | 1.00 | 59.19 | C |
| ATOM | 325 | CB | ASP | A | 76 | 77.646 | 17.265 | −8.101 | 1.00 | 59.25 | C |
| ATOM | 326 | CG | ASP | A | 76 | 77.174 | 16.004 | −7.377 | 1.00 | 60.21 | C |
| ATOM | 327 | OD1 | ASP | A | 76 | 75.950 | 15.733 | −7.378 | 1.00 | 60.76 | O |
| ATOM | 328 | OD2 | ASP | A | 76 | 77.946 | 15.218 | −6.783 | 1.00 | 61.47 | O |
| ATOM | 329 | C | ASP | A | 76 | 79.655 | 18.576 | −8.875 | 1.00 | 59.17 | C |
| ATOM | 330 | O | ASP | A | 76 | 79.536 | 19.794 | −8.702 | 1.00 | 59.08 | O |
| ATOM | 331 | N | TRP | A | 77 | 80.245 | 18.043 | −9.943 | 1.00 | 59.21 | N |
| ATOM | 332 | CA | TRP | A | 77 | 80.737 | 18.850 | −11.059 | 1.00 | 59.31 | C |
| ATOM | 333 | CB | TRP | A | 77 | 82.186 | 18.497 | −11.399 | 1.00 | 58.89 | C |
| ATOM | 334 | CG | TRP | A | 77 | 83.207 | 18.816 | −10.338 | 1.00 | 57.61 | C |
| ATOM | 335 | CD1 | TRP | A | 77 | 83.449 | 18.112 | −9.191 | 1.00 | 56.71 | C |
| ATOM | 336 | NE1 | TRP | A | 77 | 84.469 | 18.695 | −8.480 | 1.00 | 56.23 | N |
| ATOM | 337 | CE2 | TRP | A | 77 | 84.921 | 19.793 | −9.166 | 1.00 | 55.87 | C |
| ATOM | 338 | CD2 | TRP | A | 77 | 84.149 | 19.899 | −10.345 | 1.00 | 55.83 | C |
| ATOM | 339 | CE3 | TRP | A | 77 | 84.416 | 20.957 | −11.226 | 1.00 | 54.75 | C |
| ATOM | 340 | CZ3 | TRP | A | 77 | 85.429 | 21.857 | −10.909 | 1.00 | 54.49 | C |
| ATOM | 341 | CH2 | TRP | A | 77 | 86.179 | 21.722 | −9.728 | 1.00 | 54.62 | C |
| ATOM | 342 | CZ2 | TRP | A | 77 | 85.942 | 20.700 | −8.846 | 1.00 | 54.88 | C |
| ATOM | 343 | C | TRP | A | 77 | 79.880 | 18.620 | −12.297 | 1.00 | 59.97 | C |
| ATOM | 344 | O | TRP | A | 77 | 79.309 | 17.539 | −12.479 | 1.00 | 59.95 | O |
| ATOM | 345 | N | GLY | A | 78 | 79.814 | 19.636 | −13.152 | 1.00 | 60.60 | N |
| ATOM | 346 | CA | GLY | A | 78 | 79.019 | 19.573 | −14.362 | 1.00 | 61.69 | C |
| ATOM | 347 | C | GLY | A | 78 | 79.663 | 20.261 | −15.546 | 1.00 | 62.62 | C |
| ATOM | 348 | O | GLY | A | 78 | 80.722 | 20.887 | −15.425 | 1.00 | 62.53 | O |
| ATOM | 349 | N | GLU | A | 79 | 79.016 | 20.127 | −16.700 | 1.00 | 63.55 | N |
| ATOM | 350 | CA | GLU | A | 79 | 79.470 | 20.764 | −17.932 | 1.00 | 64.55 | C |
| ATOM | 351 | CB | GLU | A | 79 | 79.841 | 19.724 | −19.007 | 1.00 | 64.74 | C |
| ATOM | 352 | CG | GLU | A | 79 | 78.740 | 18.730 | −19.386 | 1.00 | 65.56 | C |
| ATOM | 353 | CD | GLU | A | 79 | 78.780 | 18.319 | −20.857 | 1.00 | 67.08 | C |
| ATOM | 354 | OE1 | GLU | A | 79 | 79.892 | 18.197 | −21.428 | 1.00 | 67.41 | O |
| ATOM | 355 | OE2 | GLU | A | 79 | 77.693 | 18.111 | −21.446 | 1.00 | 66.84 | O |
| ATOM | 356 | C | GLU | A | 79 | 78.425 | 21.745 | −18.456 | 1.00 | 64.93 | C |
| ATOM | 357 | O | GLU | A | 79 | 77.218 | 21.485 | −18.388 | 1.00 | 64.82 | O |
| ATOM | 358 | N | LEU | A | 80 | 78.902 | 22.878 | −18.963 | 1.00 | 65.56 | N |
| ATOM | 359 | CA | LEU | A | 80 | 78.039 | 23.875 | −19.589 | 1.00 | 66.20 | C |
| ATOM | 360 | CB | LEU | A | 80 | 78.763 | 25.227 | −19.663 | 1.00 | 66.19 | C |
| ATOM | 361 | CG | LEU | A | 80 | 79.128 | 25.944 | −18.359 | 1.00 | 66.08 | C |
| ATOM | 362 | CD1 | LEU | A | 80 | 79.914 | 27.225 | −18.646 | 1.00 | 65.61 | C |
| ATOM | 363 | CD2 | LEU | A | 80 | 77.881 | 26.238 | −17.525 | 1.00 | 65.99 | C |
| ATOM | 364 | C | LEU | A | 80 | 77.662 | 23.402 | −20.996 | 1.00 | 66.59 | C |
| ATOM | 365 | O | LEU | A | 80 | 78.387 | 22.585 | −21.575 | 1.00 | 66.81 | O |
| ATOM | 366 | N | PRO | A | 81 | 76.539 | 23.885 | −21.547 | 1.00 | 66.88 | N |
| ATOM | 367 | CA | PRO | A | 81 | 76.220 | 23.634 | −22.963 | 1.00 | 66.94 | C |
| ATOM | 368 | CB | PRO | A | 81 | 75.035 | 24.573 | −23.226 | 1.00 | 67.02 | C |
| ATOM | 369 | CG | PRO | A | 81 | 74.363 | 24.703 | −21.892 | 1.00 | 67.04 | C |
| ATOM | 370 | CD | PRO | A | 81 | 75.477 | 24.665 | −20.877 | 1.00 | 66.98 | C |
| ATOM | 371 | C | PRO | A | 81 | 77.408 | 23.972 | −23.884 | 1.00 | 66.80 | C |
| ATOM | 372 | O | PRO | A | 81 | 77.505 | 23.438 | −24.990 | 1.00 | 66.90 | O |
| ATOM | 373 | N | ASN | A | 82 | 78.296 | 24.842 | −23.405 | 1.00 | 66.49 | N |
| ATOM | 374 | CA | ASN | A | 82 | 79.543 | 25.182 | −24.087 | 1.00 | 66.14 | C |
| ATOM | 375 | CB | ASN | A | 82 | 80.114 | 26.482 | −23.492 | 1.00 | 66.36 | C |
| ATOM | 376 | CG | ASN | A | 82 | 81.498 | 26.816 | −24.015 | 1.00 | 67.00 | C |
| ATOM | 377 | OD1 | ASN | A | 82 | 81.734 | 26.837 | −25.225 | 1.00 | 67.52 | O |
| ATOM | 378 | ND2 | ASN | A | 82 | 82.424 | 27.089 | −23.100 | 1.00 | 67.69 | N |
| ATOM | 379 | C | ASN | A | 82 | 80.576 | 24.044 | −24.035 | 1.00 | 65.50 | C |
| ATOM | 380 | O | ASN | A | 82 | 81.273 | 23.787 | −25.019 | 1.00 | 65.53 | O |
| ATOM | 381 | N | GLY | A | 83 | 80.664 | 23.369 | −22.888 | 1.00 | 64.74 | N |
| ATOM | 382 | CA | GLY | A | 83 | 81.614 | 22.284 | −22.683 | 1.00 | 63.59 | C |
| ATOM | 383 | C | GLY | A | 83 | 82.826 | 22.690 | −21.857 | 1.00 | 62.70 | C |
| ATOM | 384 | O | GLY | A | 83 | 83.967 | 22.600 | −22.326 | 1.00 | 62.98 | O |
| ATOM | 385 | N | THR | A | 84 | 82.571 | 23.149 | −20.632 | 1.00 | 61.37 | N |
| ATOM | 386 | CA | THR | A | 84 | 83.621 | 23.529 | −19.682 | 1.00 | 59.87 | C |
| ATOM | 387 | CB | THR | A | 84 | 83.742 | 25.067 | −19.576 | 1.00 | 60.05 | C |
| ATOM | 388 | OG1 | THR | A | 84 | 83.799 | 25.643 | −20.888 | 1.00 | 60.81 | O |
| ATOM | 389 | CG2 | THR | A | 84 | 85.080 | 25.468 | −18.954 | 1.00 | 60.25 | C |
| ATOM | 390 | C | THR | A | 84 | 83.309 | 22.938 | −18.310 | 1.00 | 58.33 | C |
| ATOM | 391 | O | THR | A | 84 | 82.139 | 22.816 | −17.930 | 1.00 | 58.41 | O |
| ATOM | 392 | N | ARG | A | 85 | 84.356 | 22.576 | −17.572 | 1.00 | 56.14 | N |
| ATOM | 393 | CA | ARG | A | 85 | 84.198 | 22.026 | −16.231 | 1.00 | 53.98 | C |
| ATOM | 394 | CB | ARG | A | 85 | 85.445 | 21.223 | −15.844 | 1.00 | 54.58 | C |
| ATOM | 395 | CG | ARG | A | 85 | 85.227 | 20.243 | −14.703 | 1.00 | 56.25 | C |
| ATOM | 396 | CD | ARG | A | 85 | 86.028 | 18.945 | −14.819 | 1.00 | 58.89 | C |
| ATOM | 397 | NE | ARG | A | 85 | 85.870 | 18.099 | −13.630 | 1.00 | 60.27 | N |
| ATOM | 398 | CZ | ARG | A | 85 | 84.879 | 17.226 | −13.445 | 1.00 | 60.85 | C |
| ATOM | 399 | NH1 | ARG | A | 85 | 83.933 | 17.065 | −14.370 | 1.00 | 61.04 | N |
| ATOM | 400 | NH2 | ARG | A | 85 | 84.834 | 16.506 | −12.329 | 1.00 | 60.48 | N |
| ATOM | 401 | C | ARG | A | 85 | 83.906 | 23.130 | −15.200 | 1.00 | 51.86 | C |
| ATOM | 402 | O | ARG | A | 85 | 84.789 | 23.931 | −14.865 | 1.00 | 51.74 | O |
| ATOM | 403 | N | VAL | A | 86 | 82.659 | 23.172 | −14.721 | 1.00 | 48.87 | N |
| ATOM | 404 | CA | VAL | A | 86 | 82.224 | 24.121 | −13.680 | 1.00 | 45.97 | C |
| ATOM | 405 | CB | VAL | A | 86 | 81.335 | 25.276 | −14.251 | 1.00 | 46.05 | C |
| ATOM | 406 | CG1 | VAL | A | 86 | 82.074 | 26.064 | −15.322 | 1.00 | 46.41 | C |
| ATOM | 407 | CG2 | VAL | A | 86 | 80.008 | 24.755 | −14.781 | 1.00 | 45.90 | C |
| ATOM | 408 | C | VAL | A | 86 | 81.462 | 23.409 | −12.553 | 1.00 | 43.56 | C |
| ATOM | 409 | O | VAL | A | 86 | 80.984 | 22.292 | −12.750 | 1.00 | 43.24 | O |
| ATOM | 410 | N | PRO | A | 87 | 81.345 | 24.040 | −11.380 | 1.00 | 41.11 | N |
| ATOM | 411 | CA | PRO | A | 87 | 80.494 | 23.495 | −10.314 | 1.00 | 39.00 | C |
| ATOM | 412 | CB | PRO | A | 87 | 80.654 | 24.499 | −9.158 | 1.00 | 39.06 | C |
| ATOM | 413 | CG | PRO | A | 87 | 81.251 | 25.719 | −9.759 | 1.00 | 40.31 | C |
| ATOM | 414 | CD | PRO | A | 87 | 82.015 | 25.287 | −10.966 | 1.00 | 40.95 | C |
| ATOM | 415 | C | PRO | A | 87 | 79.037 | 23.413 | −10.755 | 1.00 | 36.73 | C |
| ATOM | 416 | O | PRO | A | 87 | 78.567 | 24.258 | −11.535 | 1.00 | 35.64 | O |
| ATOM | 417 | N | MET | A | 88 | 78.343 | 22.391 | −10.255 | 1.00 | 34.56 | N |
| ATOM | 418 | CA | MET | A | 88 | 76.936 | 22.171 | −10.564 | 1.00 | 32.47 | C |
| ATOM | 419 | CB | MET | A | 88 | 76.408 | 20.950 | −9.799 | 1.00 | 33.23 | C |
| ATOM | 420 | CG | MET | A | 88 | 75.047 | 20.407 | −10.263 | 1.00 | 36.09 | C |
| ATOM | 421 | SD | MET | A | 88 | 74.917 | 19.957 | −12.033 | 1.00 | 43.02 | S |
| ATOM | 422 | CE | MET | A | 88 | 76.260 | 18.799 | −12.218 | 1.00 | 41.30 | C |
| ATOM | 423 | C | MET | A | 88 | 76.110 | 23.423 | −10.274 | 1.00 | 30.24 | C |
| ATOM | 424 | O | MET | A | 88 | 75.169 | 23.717 | −11.006 | 1.00 | 29.10 | O |
| ATOM | 425 | N | GLU | A | 89 | 76.487 | 24.169 | −9.231 | 1.00 | 28.21 | N |
| ATOM | 426 | CA | GLU | A | 89 | 75.773 | 25.392 | −8.843 | 1.00 | 26.55 | C |
| ATOM | 427 | CB | GLU | A | 89 | 76.393 | 26.043 | −7.576 | 1.00 | 26.83 | C |
| ATOM | 428 | CG | GLU | A | 89 | 75.711 | 27.347 | −7.134 | 1.00 | 27.21 | C |
| ATOM | 429 | CD | GLU | A | 89 | 75.939 | 27.733 | −5.670 | 1.00 | 29.78 | C |
| ATOM | 430 | OE1 | GLU | A | 89 | 76.956 | 27.292 | −5.073 | 1.00 | 29.01 | O |
| ATOM | 431 | OE2 | GLU | A | 89 | 75.080 | 28.483 | −5.118 | 1.00 | 29.29 | O |
| ATOM | 432 | C | GLU | A | 89 | 75.669 | 26.392 | −10.000 | 1.00 | 25.41 | C |
| ATOM | 433 | O | GLU | A | 89 | 74.609 | 26.988 | −10.223 | 1.00 | 25.07 | O |
| ATOM | 434 | N | VAL | A | 90 | 76.761 | 26.566 | −10.744 | 1.00 | 24.25 | N |
| ATOM | 435 | CA | VAL | A | 90 | 76.747 | 27.435 | −11.927 | 1.00 | 23.34 | C |
| ATOM | 436 | CB | VAL | A | 90 | 78.193 | 27.717 | −12.452 | 1.00 | 23.97 | C |
| ATOM | 437 | CG1 | VAL | A | 90 | 78.178 | 28.460 | −13.796 | 1.00 | 23.00 | C |
| ATOM | 438 | CG2 | VAL | A | 90 | 78.989 | 28.530 | −11.411 | 1.00 | 23.11 | C |
| ATOM | 439 | C | VAL | A | 90 | 75.822 | 26.881 | −13.020 | 1.00 | 22.89 | C |
| ATOM | 440 | O | VAL | A | 90 | 75.002 | 27.622 | −13.570 | 1.00 | 22.78 | O |
| ATOM | 441 | N | VAL | A | 91 | 75.926 | 25.579 | −13.305 | 1.00 | 22.51 | N |
| ATOM | 442 | CA | VAL | A | 91 | 75.048 | 24.917 | −14.293 | 1.00 | 22.19 | C |
| ATOM | 443 | CB | VAL | A | 91 | 75.316 | 23.382 | −14.399 | 1.00 | 22.50 | C |
| ATOM | 444 | CG1 | VAL | A | 91 | 74.265 | 22.688 | −15.314 | 1.00 | 22.61 | C |
| ATOM | 445 | CG2 | VAL | A | 91 | 76.688 | 23.116 | −14.934 | 1.00 | 22.68 | C |
| ATOM | 446 | C | VAL | A | 91 | 73.569 | 25.143 | −13.965 | 1.00 | 21.66 | C |
| ATOM | 447 | O | VAL | A | 91 | 72.783 | 25.594 | −14.807 | 1.00 | 20.36 | O |
| ATOM | 448 | N | LEU | A | 92 | 73.215 | 24.856 | −12.715 | 1.00 | 21.61 | N |
| ATOM | 449 | CA | LEU | A | 92 | 71.833 | 24.963 | −12.256 | 1.00 | 21.57 | C |
| ATOM | 450 | CB | LEU | A | 92 | 71.682 | 24.326 | −10.873 | 1.00 | 21.14 | C |
| ATOM | 451 | CG | LEU | A | 92 | 72.112 | 22.854 | −10.778 | 1.00 | 21.50 | C |
| ATOM | 452 | CD1 | LEU | A | 92 | 71.945 | 22.365 | −9.349 | 1.00 | 20.58 | C |
| ATOM | 453 | CD2 | LEU | A | 92 | 71.378 | 21.928 | −11.767 | 1.00 | 18.41 | C |
| ATOM | 454 | C | LEU | A | 92 | 71.331 | 26.408 | −12.255 | 1.00 | 21.79 | C |
| ATOM | 455 | O | LEU | A | 92 | 70.212 | 26.671 | −12.706 | 1.00 | 21.50 | O |
| ATOM | 456 | N | LEU | A | 93 | 72.159 | 27.332 | −11.764 | 1.00 | 22.36 | N |
| ATOM | 457 | CA | LEU | A | 93 | 71.808 | 28.755 | −11.756 | 1.00 | 23.34 | C |
| ATOM | 458 | CB | LEU | A | 93 | 72.861 | 29.584 | −11.018 | 1.00 | 23.50 | C |
| ATOM | 459 | CG | LEU | A | 93 | 72.714 | 29.608 | −9.482 | 1.00 | 24.22 | C |
| ATOM | 460 | CD1 | LEU | A | 93 | 73.985 | 30.131 | −8.852 | 1.00 | 23.74 | C |
| ATOM | 461 | CD2 | LEU | A | 93 | 71.493 | 30.431 | −9.048 | 1.00 | 22.10 | C |
| ATOM | 462 | C | LEU | A | 93 | 71.594 | 29.301 | −13.162 | 1.00 | 23.97 | C |
| ATOM | 463 | O | LEU | A | 93 | 70.642 | 30.037 | −13.409 | 1.00 | 23.67 | O |
| ATOM | 464 | N | LYS | A | 94 | 72.468 | 28.922 | −14.088 | 1.00 | 24.87 | N |
| ATOM | 465 | CA | LYS | A | 94 | 72.265 | 29.290 | −15.491 | 1.00 | 26.26 | C |
| ATOM | 466 | CB | LYS | A | 94 | 73.448 | 28.847 | −16.356 | 1.00 | 26.36 | C |
| ATOM | 467 | CG | LYS | A | 94 | 74.664 | 29.745 | −16.166 | 1.00 | 29.54 | C |
| ATOM | 468 | CD | LYS | A | 94 | 75.932 | 29.132 | −16.750 | 1.00 | 34.13 | C |
| ATOM | 469 | CE | LYS | A | 94 | 76.210 | 29.633 | −18.167 | 1.00 | 36.90 | C |
| ATOM | 470 | NZ | LYS | A | 94 | 76.658 | 31.059 | −18.181 | 1.00 | 39.00 | N |
| ATOM | 471 | C | LYS | A | 94 | 70.946 | 28.761 | −16.045 | 1.00 | 26.31 | C |
| ATOM | 472 | O | LYS | A | 94 | 70.240 | 29.481 | −16.756 | 1.00 | 26.21 | O |
| ATOM | 473 | N | LYS | A | 95 | 70.610 | 27.515 | −15.712 | 1.00 | 26.64 | N |
| ATOM | 474 | CA | LYS | A | 95 | 69.356 | 26.916 | −16.172 | 1.00 | 28.04 | C |
| ATOM | 475 | CB | LYS | A | 95 | 69.295 | 25.427 | −15.824 | 1.00 | 27.38 | C |
| ATOM | 476 | CG | LYS | A | 95 | 70.096 | 24.557 | −16.777 | 1.00 | 28.63 | C |
| ATOM | 477 | CD | LYS | A | 95 | 70.218 | 23.114 | −16.294 | 1.00 | 29.42 | C |
| ATOM | 478 | CE | LYS | A | 95 | 68.891 | 22.361 | −16.392 | 1.00 | 30.74 | C |
| ATOM | 479 | NZ | LYS | A | 95 | 68.513 | 22.039 | −17.803 | 1.00 | 31.02 | N |
| ATOM | 480 | C | LYS | A | 95 | 68.096 | 27.657 | −15.674 | 1.00 | 28.66 | C |
| ATOM | 481 | O | LYS | A | 95 | 67.088 | 27.707 | −16.379 | 1.00 | 28.72 | O |
| ATOM | 482 | N | VAL | A | 96 | 68.167 | 28.239 | −14.480 | 1.00 | 30.05 | N |
| ATOM | 483 | CA | VAL | A | 96 | 67.017 | 28.940 | −13.899 | 1.00 | 31.67 | C |
| ATOM | 484 | CB | VAL | A | 96 | 66.818 | 28.631 | −12.383 | 1.00 | 31.32 | C |
| ATOM | 485 | CG1 | VAL | A | 96 | 66.594 | 27.139 | −12.159 | 1.00 | 30.44 | C |
| ATOM | 486 | CG2 | VAL | A | 96 | 67.978 | 29.149 | −11.546 | 1.00 | 29.93 | C |
| ATOM | 487 | C | VAL | A | 96 | 66.997 | 30.458 | −14.119 | 1.00 | 33.63 | C |
| ATOM | 488 | O | VAL | A | 96 | 65.999 | 31.112 | −13.783 | 1.00 | 33.80 | O |
| ATOM | 489 | N | SER | A | 97 | 68.074 | 31.018 | −14.676 | 1.00 | 35.26 | N |
| ATOM | 490 | CA | SER | A | 97 | 68.109 | 32.455 | −14.979 | 1.00 | 37.17 | C |
| ATOM | 491 | CB | SER | A | 97 | 69.490 | 32.907 | −15.483 | 1.00 | 37.37 | C |
| ATOM | 492 | OG | SER | A | 97 | 69.844 | 32.265 | −16.699 | 1.00 | 38.96 | O |
| ATOM | 493 | C | SER | A | 97 | 67.009 | 32.865 | −15.962 | 1.00 | 38.05 | C |
| ATOM | 494 | O | SER | A | 97 | 66.797 | 32.223 | −16.996 | 1.00 | 38.07 | O |
| ATOM | 495 | N | SER | A | 98 | 66.302 | 33.934 | −15.603 | 1.00 | 39.50 | N |
| ATOM | 496 | CA | SER | A | 98 | 65.224 | 34.521 | −16.409 | 1.00 | 40.49 | C |
| ATOM | 497 | CB | SER | A | 98 | 64.109 | 33.499 | −16.685 | 1.00 | 40.48 | C |
| ATOM | 498 | OG | SER | A | 98 | 63.105 | 33.547 | −15.681 | 1.00 | 41.42 | O |
| ATOM | 499 | C | SER | A | 98 | 64.671 | 35.738 | −15.656 | 1.00 | 40.82 | C |
| ATOM | 500 | O | SER | A | 98 | 65.177 | 36.091 | −14.582 | 1.00 | 41.27 | O |
| ATOM | 501 | N | GLY | A | 99 | 63.632 | 36.364 | −16.210 | 1.00 | 40.95 | N |
| ATOM | 502 | CA | GLY | A | 99 | 63.015 | 37.535 | −15.602 | 1.00 | 40.62 | C |
| ATOM | 503 | C | GLY | A | 99 | 62.281 | 37.294 | −14.283 | 1.00 | 40.18 | C |
| ATOM | 504 | O | GLY | A | 99 | 61.912 | 38.263 | −13.600 | 1.00 | 40.34 | O |
| ATOM | 505 | N | PHE | A | 100 | 62.056 | 36.019 | −13.942 | 1.00 | 39.30 | N |
| ATOM | 506 | CA | PHE | A | 100 | 61.391 | 35.628 | −12.694 | 1.00 | 38.13 | C |
| ATOM | 507 | CB | PHE | A | 100 | 61.038 | 34.135 | −12.709 | 1.00 | 38.35 | C |
| ATOM | 508 | CG | PHE | A | 100 | 60.296 | 33.656 | −11.471 | 1.00 | 37.90 | C |
| ATOM | 509 | CD1 | PHE | A | 100 | 59.100 | 34.258 | −11.069 | 1.00 | 36.99 | C |
| ATOM | 510 | CE1 | PHE | A | 100 | 58.411 | 33.801 | −9.924 | 1.00 | 37.18 | C |
| ATOM | 511 | CZ | PHE | A | 100 | 58.922 | 32.727 | −9.177 | 1.00 | 35.96 | C |
| ATOM | 512 | CE2 | PHE | A | 100 | 60.108 | 32.116 | −9.573 | 1.00 | 35.95 | C |
| ATOM | 513 | CD2 | PHE | A | 100 | 60.792 | 32.585 | −10.718 | 1.00 | 37.98 | C |
| ATOM | 514 | C | PHE | A | 100 | 62.266 | 35.944 | −11.491 | 1.00 | 37.35 | C |
| ATOM | 515 | O | PHE | A | 100 | 63.365 | 35.409 | −11.354 | 1.00 | 37.45 | O |
| ATOM | 516 | N | SER | A | 101 | 61.768 | 36.814 | −10.620 | 1.00 | 36.17 | N |
| ATOM | 517 | CA | SER | A | 101 | 62.534 | 37.263 | −9.468 | 1.00 | 35.23 | C |
| ATOM | 518 | CB | SER | A | 101 | 62.048 | 38.647 | −9.003 | 1.00 | 35.81 | C |
| ATOM | 519 | OG | SER | A | 101 | 60.697 | 38.612 | −8.570 | 1.00 | 37.04 | O |
| ATOM | 520 | C | SER | A | 101 | 62.571 | 36.280 | −8.291 | 1.00 | 33.45 | C |
| ATOM | 521 | O | SER | A | 101 | 63.260 | 36.544 | −7.295 | 1.00 | 33.93 | O |
| ATOM | 522 | N | GLY | A | 102 | 61.856 | 35.157 | −8.402 | 1.00 | 31.13 | N |
| ATOM | 523 | CA | GLY | A | 102 | 61.760 | 34.190 | −7.310 | 1.00 | 28.02 | C |
| ATOM | 524 | C | GLY | A | 102 | 63.026 | 33.377 | −7.066 | 1.00 | 26.06 | C |
| ATOM | 525 | O | GLY | A | 102 | 63.183 | 32.736 | −6.040 | 1.00 | 24.79 | O |
| ATOM | 526 | N | VAL | A | 103 | 63.936 | 33.396 | −8.030 | 1.00 | 25.16 | N |
| ATOM | 527 | CA | VAL | A | 103 | 65.213 | 32.726 | −7.877 | 1.00 | 24.40 | C |
| ATOM | 528 | CB | VAL | A | 103 | 65.377 | 31.540 | −8.863 | 1.00 | 24.34 | C |
| ATOM | 529 | CG1 | VAL | A | 103 | 66.675 | 30.797 | −8.585 | 1.00 | 25.25 | C |
| ATOM | 530 | CG2 | VAL | A | 103 | 64.214 | 30.567 | −8.737 | 1.00 | 23.66 | C |
| ATOM | 531 | C | VAL | A | 103 | 66.300 | 33.759 | −8.104 | 1.00 | 24.50 | C |
| ATOM | 532 | O | VAL | A | 103 | 66.217 | 34.566 | −9.040 | 1.00 | 24.27 | O |
| ATOM | 533 | N | ILE | A | 104 | 67.303 | 33.744 | −7.232 | 1.00 | 23.78 | N |
| ATOM | 534 | CA | ILE | A | 104 | 68.477 | 34.576 | −7.383 | 1.00 | 24.18 | C |
| ATOM | 535 | CB | ILE | A | 104 | 69.526 | 34.185 | −6.324 | 1.00 | 24.30 | C |
| ATOM | 536 | CG1 | ILE | A | 104 | 70.384 | 35.394 | −5.954 | 1.00 | 23.11 | C |
| ATOM | 537 | CD1 | ILE | A | 104 | 69.581 | 36.449 | −5.162 | 1.00 | 22.00 | C |
| ATOM | 538 | CG2 | ILE | A | 104 | 70.327 | 32.934 | −6.766 | 1.00 | 23.91 | C |
| ATOM | 539 | C | ILE | A | 104 | 69.083 | 34.483 | −8.789 | 1.00 | 24.77 | C |
| ATOM | 540 | O | ILE | A | 104 | 69.188 | 33.403 | −9.366 | 1.00 | 25.02 | O |
| ATOM | 541 | N | ARG | A | 105 | 69.479 | 35.619 | −9.337 | 1.00 | 25.08 | N |
| ATOM | 542 | CA | ARG | A | 105 | 70.070 | 35.622 | −10.667 | 1.00 | 26.17 | C |
| ATOM | 543 | CB | ARG | A | 105 | 69.566 | 36.833 | −11.454 | 1.00 | 27.31 | C |
| ATOM | 544 | CG | ARG | A | 105 | 70.349 | 37.173 | −12.714 | 1.00 | 32.33 | C |
| ATOM | 545 | CD | ARG | A | 105 | 69.728 | 38.311 | −13.536 | 1.00 | 39.80 | C |
| ATOM | 546 | NE | ARG | A | 105 | 68.331 | 38.040 | −13.891 | 1.00 | 45.22 | N |
| ATOM | 547 | CZ | ARG | A | 105 | 67.573 | 38.838 | −14.646 | 1.00 | 47.93 | C |
| ATOM | 548 | NH1 | ARG | A | 105 | 68.062 | 39.976 | −15.139 | 1.00 | 48.73 | N |
| ATOM | 549 | NH2 | ARG | A | 105 | 66.319 | 38.498 | −14.908 | 1.00 | 48.97 | N |
| ATOM | 550 | C | ARG | A | 105 | 71.593 | 35.590 | −10.594 | 1.00 | 25.19 | C |
| ATOM | 551 | O | ARG | A | 105 | 72.211 | 36.402 | −9.885 | 1.00 | 24.65 | O |
| ATOM | 552 | N | LEU | A | 106 | 72.188 | 34.634 | −11.304 | 1.00 | 24.85 | N |
| ATOM | 553 | CA | LEU | A | 106 | 73.642 | 34.609 | −11.499 | 1.00 | 24.90 | C |
| ATOM | 554 | CB | LEU | A | 106 | 74.136 | 33.223 | −11.918 | 1.00 | 24.41 | C |
| ATOM | 555 | CG | LEU | A | 106 | 75.651 | 33.073 | −12.127 | 1.00 | 24.89 | C |
| ATOM | 556 | CD1 | LEU | A | 106 | 76.449 | 33.148 | −10.796 | 1.00 | 24.67 | C |
| ATOM | 557 | CD2 | LEU | A | 106 | 75.961 | 31.790 | −12.871 | 1.00 | 23.97 | C |
| ATOM | 558 | C | LEU | A | 106 | 74.004 | 35.639 | −12.554 | 1.00 | 25.16 | C |
| ATOM | 559 | O | LEU | A | 106 | 73.536 | 35.565 | −13.695 | 1.00 | 25.41 | O |
| ATOM | 560 | N | LEU | A | 107 | 74.825 | 36.604 | −12.163 | 1.00 | 25.22 | N |
| ATOM | 561 | CA | LEU | A | 107 | 75.217 | 37.703 | −13.046 | 1.00 | 25.72 | C |
| ATOM | 562 | CB | LEU | A | 107 | 75.427 | 38.991 | −12.240 | 1.00 | 25.54 | C |
| ATOM | 563 | CG | LEU | A | 107 | 74.167 | 39.501 | −11.524 | 1.00 | 25.95 | C |
| ATOM | 564 | CD1 | LEU | A | 107 | 74.478 | 40.685 | −10.620 | 1.00 | 25.00 | C |
| ATOM | 565 | CD2 | LEU | A | 107 | 73.067 | 39.868 | −12.525 | 1.00 | 27.51 | C |
| ATOM | 566 | C | LEU | A | 107 | 76.465 | 37.363 | −13.847 | 1.00 | 25.66 | C |
| ATOM | 567 | O | LEU | A | 107 | 76.553 | 37.678 | −15.040 | 1.00 | 25.68 | O |
| ATOM | 568 | N | ASP | A | 108 | 77.420 | 36.717 | −13.177 | 1.00 | 25.47 | N |
| ATOM | 569 | CA | ASP | A | 108 | 78.699 | 36.333 | −13.762 | 1.00 | 25.47 | C |
| ATOM | 570 | CB | ASP | A | 108 | 79.624 | 37.557 | −13.872 | 1.00 | 25.78 | C |
| ATOM | 571 | CG | ASP | A | 108 | 80.569 | 37.485 | −15.071 | 1.00 | 27.00 | C |
| ATOM | 572 | OD1 | ASP | A | 108 | 80.828 | 36.385 | −15.610 | 1.00 | 27.45 | O |
| ATOM | 573 | OD2 | ASP | A | 108 | 81.111 | 38.501 | −15.537 | 1.00 | 29.70 | O |
| ATOM | 574 | C | ASP | A | 108 | 79.358 | 35.308 | −12.856 | 1.00 | 25.21 | C |
| ATOM | 575 | O | ASP | A | 108 | 78.938 | 35.119 | −11.711 | 1.00 | 23.96 | O |
| ATOM | 576 | N | TRP | A | 109 | 80.405 | 34.669 | −13.369 | 1.00 | 25.09 | N |
| ATOM | 577 | CA | TRP | A | 109 | 81.235 | 33.785 | −12.565 | 1.00 | 25.83 | C |
| ATOM | 578 | CB | TRP | A | 109 | 80.668 | 32.360 | −12.565 | 1.00 | 26.03 | C |
| ATOM | 579 | CG | TRP | A | 109 | 80.690 | 31.729 | −13.918 | 1.00 | 27.82 | C |
| ATOM | 580 | CD1 | TRP | A | 109 | 79.727 | 31.818 | −14.883 | 1.00 | 28.39 | C |
| ATOM | 581 | NE1 | TRP | A | 109 | 80.102 | 31.102 | −15.995 | 1.00 | 30.13 | N |
| ATOM | 582 | CE2 | TRP | A | 109 | 81.332 | 30.539 | −15.770 | 1.00 | 30.67 | C |
| ATOM | 583 | CD2 | TRP | A | 109 | 81.732 | 30.914 | −14.465 | 1.00 | 29.83 | C |
| ATOM | 584 | CE3 | TRP | A | 109 | 82.970 | 30.460 | −13.987 | 1.00 | 30.76 | C |
| ATOM | 585 | CZ3 | TRP | A | 109 | 83.758 | 29.664 | −14.809 | 1.00 | 32.48 | C |
| ATOM | 586 | CH2 | TRP | A | 109 | 83.334 | 29.313 | −16.107 | 1.00 | 33.06 | C |
| ATOM | 587 | CZ2 | TRP | A | 109 | 82.126 | 29.739 | −16.602 | 1.00 | 32.39 | C |
| ATOM | 588 | C | TRP | A | 109 | 82.689 | 33.808 | −13.045 | 1.00 | 26.10 | C |
| ATOM | 589 | O | TRP | A | 109 | 82.973 | 34.170 | −14.191 | 1.00 | 25.61 | O |
| ATOM | 590 | N | PHE | A | 110 | 83.599 | 33.425 | −12.155 | 1.00 | 26.36 | N |
| ATOM | 591 | CA | PHE | A | 110 | 85.028 | 33.407 | −12.449 | 1.00 | 26.90 | C |
| ATOM | 592 | CB | PHE | A | 110 | 85.734 | 34.607 | −11.796 | 1.00 | 27.07 | C |
| ATOM | 593 | CG | PHE | A | 110 | 85.249 | 35.945 | −12.285 | 1.00 | 28.66 | C |
| ATOM | 594 | CD1 | PHE | A | 110 | 85.909 | 36.602 | −13.330 | 1.00 | 30.61 | C |
| ATOM | 595 | CE1 | PHE | A | 110 | 85.464 | 37.851 | −13.785 | 1.00 | 31.43 | C |
| ATOM | 596 | CZ | PHE | A | 110 | 84.344 | 38.446 | −13.195 | 1.00 | 31.46 | C |
| ATOM | 597 | CE2 | PHE | A | 110 | 83.679 | 37.795 | −12.153 | 1.00 | 30.52 | C |
| ATOM | 598 | CD2 | PHE | A | 110 | 84.140 | 36.556 | −11.701 | 1.00 | 28.63 | C |
| ATOM | 599 | C | PHE | A | 110 | 85.646 | 32.117 | −11.924 | 1.00 | 27.02 | C |
| ATOM | 600 | O | PHE | A | 110 | 85.227 | 31.591 | −10.879 | 1.00 | 26.50 | O |
| ATOM | 601 | N | GLU | A | 111 | 86.638 | 31.614 | −12.655 | 1.00 | 26.99 | N |
| ATOM | 602 | CA | GLU | A | 111 | 87.454 | 30.500 | −12.201 | 1.00 | 27.50 | C |
| ATOM | 603 | CB | GLU | A | 111 | 87.683 | 29.476 | −13.323 | 1.00 | 27.99 | C |
| ATOM | 604 | CG | GLU | A | 111 | 88.309 | 28.179 | −12.828 | 1.00 | 28.36 | C |
| ATOM | 605 | CD | GLU | A | 111 | 88.468 | 27.116 | −13.894 | 1.00 | 29.75 | C |
| ATOM | 606 | OE1 | GLU | A | 111 | 87.864 | 27.215 | −14.989 | 1.00 | 31.47 | O |
| ATOM | 607 | OE2 | GLU | A | 111 | 89.206 | 26.154 | −13.622 | 1.00 | 30.33 | O |
| ATOM | 608 | C | GLU | A | 111 | 88.796 | 31.023 | −11.696 | 1.00 | 27.98 | C |
| ATOM | 609 | O | GLU | A | 111 | 89.415 | 31.891 | −12.310 | 1.00 | 28.26 | O |
| ATOM | 610 | N | ARG | A | 112 | 89.225 | 30.490 | −10.560 | 1.00 | 28.31 | N |
| ATOM | 611 | CA | ARG | A | 112 | 90.541 | 30.760 | −10.004 | 1.00 | 28.18 | C |
| ATOM | 612 | CB | ARG | A | 112 | 90.403 | 31.378 | −8.614 | 1.00 | 28.37 | C |
| ATOM | 613 | CG | ARG | A | 112 | 90.263 | 32.883 | −8.622 | 1.00 | 27.57 | C |
| ATOM | 614 | CD | ARG | A | 112 | 89.828 | 33.452 | −7.293 | 1.00 | 27.27 | C |
| ATOM | 615 | NE | ARG | A | 112 | 89.976 | 34.899 | −7.282 | 1.00 | 26.93 | N |
| ATOM | 616 | CZ | ARG | A | 112 | 89.758 | 35.671 | −6.233 | 1.00 | 27.51 | C |
| ATOM | 617 | NH1 | ARG | A | 112 | 89.360 | 35.146 | −5.079 | 1.00 | 28.76 | N |
| ATOM | 618 | NH2 | ARG | A | 112 | 89.932 | 36.979 | −6.339 | 1.00 | 26.92 | N |
| ATOM | 619 | C | ARG | A | 112 | 91.255 | 29.413 | −9.933 | 1.00 | 28.60 | C |
| ATOM | 620 | O | ARG | A | 112 | 90.627 | 28.379 | −10.197 | 1.00 | 28.00 | O |
| ATOM | 621 | N | PRO | A | 113 | 92.556 | 29.402 | −9.616 | 1.00 | 28.77 | N |
| ATOM | 622 | CA | PRO | A | 113 | 93.282 | 28.129 | −9.517 | 1.00 | 28.93 | C |
| ATOM | 623 | CB | PRO | A | 113 | 94.697 | 28.557 | −9.102 | 1.00 | 29.22 | C |
| ATOM | 624 | CG | PRO | A | 113 | 94.817 | 29.977 | −9.608 | 1.00 | 29.25 | C |
| ATOM | 625 | CD | PRO | A | 113 | 93.444 | 30.560 | −9.383 | 1.00 | 28.81 | C |
| ATOM | 626 | C | PRO | A | 113 | 92.642 | 27.163 | −8.505 | 1.00 | 28.63 | C |
| ATOM | 627 | O | PRO | A | 113 | 92.473 | 25.982 | −8.829 | 1.00 | 28.81 | O |
| ATOM | 628 | N | ASP | A | 114 | 92.239 | 27.664 | −7.340 | 1.00 | 28.09 | N |
| ATOM | 629 | CA | ASP | A | 114 | 91.740 | 26.800 | −6.269 | 1.00 | 27.57 | C |
| ATOM | 630 | CB | ASP | A | 114 | 92.605 | 26.991 | −5.020 | 1.00 | 28.11 | C |
| ATOM | 631 | CG | ASP | A | 114 | 94.078 | 26.644 | −5.272 | 1.00 | 30.45 | C |
| ATOM | 632 | OD1 | ASP | A | 114 | 94.959 | 27.360 | −4.740 | 1.00 | 31.83 | O |
| ATOM | 633 | OD2 | ASP | A | 114 | 94.438 | 25.680 | −5.998 | 1.00 | 30.94 | O |
| ATOM | 634 | C | ASP | A | 114 | 90.252 | 26.962 | −5.921 | 1.00 | 26.62 | C |
| ATOM | 635 | O | ASP | A | 114 | 89.754 | 26.323 | −4.980 | 1.00 | 26.49 | O |
| ATOM | 636 | N | SER | A | 115 | 89.549 | 27.806 | −6.677 | 1.00 | 25.25 | N |
| ATOM | 637 | CA | SER | A | 115 | 88.150 | 28.130 | −6.382 | 1.00 | 23.81 | C |
| ATOM | 638 | CB | SER | A | 115 | 88.100 | 29.182 | −5.276 | 1.00 | 23.83 | C |
| ATOM | 639 | OG | SER | A | 115 | 88.650 | 30.403 | −5.733 | 1.00 | 22.52 | O |
| ATOM | 640 | C | SER | A | 115 | 87.352 | 28.653 | −7.586 | 1.00 | 23.20 | C |
| ATOM | 641 | O | SER | A | 115 | 87.917 | 28.964 | −8.639 | 1.00 | 22.61 | O |
| ATOM | 642 | N | PHE | A | 116 | 86.039 | 28.761 | −7.400 | 1.00 | 22.13 | N |
| ATOM | 643 | CA | PHE | A | 116 | 85.175 | 29.515 | −8.306 | 1.00 | 21.81 | C |
| ATOM | 644 | CB | PHE | A | 116 | 84.074 | 28.627 | −8.901 | 1.00 | 21.79 | C |
| ATOM | 645 | CG | PHE | A | 116 | 84.578 | 27.655 | −9.921 | 1.00 | 22.56 | C |
| ATOM | 646 | CD1 | PHE | A | 116 | 85.096 | 26.425 | −9.532 | 1.00 | 23.50 | C |
| ATOM | 647 | CE1 | PHE | A | 116 | 85.581 | 25.515 | −10.487 | 1.00 | 25.19 | C |
| ATOM | 648 | CZ | PHE | A | 116 | 85.550 | 25.846 | −11.835 | 1.00 | 24.86 | C |
| ATOM | 649 | CE2 | PHE | A | 116 | 85.032 | 27.080 | −12.230 | 1.00 | 25.05 | C |
| ATOM | 650 | CD2 | PHE | A | 116 | 84.551 | 27.974 | −11.271 | 1.00 | 24.09 | C |
| ATOM | 651 | C | PHE | A | 116 | 84.556 | 30.678 | −7.553 | 1.00 | 21.29 | C |
| ATOM | 652 | O | PHE | A | 116 | 84.349 | 30.605 | −6.341 | 1.00 | 21.62 | O |
| ATOM | 653 | N | VAL | A | 117 | 84.280 | 31.757 | −8.275 | 1.00 | 20.75 | N |
| ATOM | 654 | CA | VAL | A | 117 | 83.674 | 32.944 | −7.707 | 1.00 | 19.94 | C |
| ATOM | 655 | CB | VAL | A | 117 | 84.628 | 34.134 | −7.791 | 1.00 | 20.10 | C |
| ATOM | 656 | CG1 | VAL | A | 117 | 84.036 | 35.341 | −7.089 | 1.00 | 19.06 | C |
| ATOM | 657 | CG2 | VAL | A | 117 | 86.019 | 33.768 | −7.189 | 1.00 | 20.50 | C |
| ATOM | 658 | C | VAL | A | 117 | 82.399 | 33.242 | −8.489 | 1.00 | 19.85 | C |
| ATOM | 659 | O | VAL | A | 117 | 82.441 | 33.393 | −9.713 | 1.00 | 20.09 | O |
| ATOM | 660 | N | LEU | A | 118 | 81.276 | 33.327 | −7.785 | 1.00 | 19.32 | N |
| ATOM | 661 | CA | LEU | A | 118 | 79.981 | 33.584 | −8.400 | 1.00 | 19.32 | C |
| ATOM | 662 | CB | LEU | A | 118 | 78.936 | 32.565 | −7.912 | 1.00 | 19.30 | C |
| ATOM | 663 | CG | LEU | A | 118 | 78.914 | 31.157 | −8.510 | 1.00 | 20.91 | C |
| ATOM | 664 | CD1 | LEU | A | 118 | 80.203 | 30.381 | −8.224 | 1.00 | 23.74 | C |
| ATOM | 665 | CD2 | LEU | A | 118 | 77.741 | 30.382 | −7.949 | 1.00 | 22.22 | C |
| ATOM | 666 | C | LEU | A | 118 | 79.514 | 34.981 | −8.051 | 1.00 | 19.31 | C |
| ATOM | 667 | O | LEU | A | 118 | 79.574 | 35.387 | −6.887 | 1.00 | 19.13 | O |
| ATOM | 668 | N | ILE | A | 119 | 79.048 | 35.715 | −9.062 | 1.00 | 19.23 | N |
| ATOM | 669 | CA | ILE | A | 119 | 78.521 | 37.053 | −8.860 | 1.00 | 19.06 | C |
| ATOM | 670 | CB | ILE | A | 119 | 79.093 | 38.062 | −9.898 | 1.00 | 19.52 | C |
| ATOM | 671 | CG1 | ILE | A | 119 | 80.627 | 37.931 | −10.022 | 1.00 | 19.59 | C |
| ATOM | 672 | CD1 | ILE | A | 119 | 81.434 | 38.222 | −8.736 | 1.00 | 19.03 | C |
| ATOM | 673 | CG2 | ILE | A | 119 | 78.652 | 39.509 | −9.557 | 1.00 | 18.88 | C |
| ATOM | 674 | C | ILE | A | 119 | 77.008 | 36.958 | −8.938 | 1.00 | 19.43 | C |
| ATOM | 675 | O | ILE | A | 119 | 76.446 | 36.592 | −9.977 | 1.00 | 19.01 | O |
| ATOM | 676 | N | LEU | A | 120 | 76.358 | 37.266 | −7.823 | 1.00 | 19.78 | N |
| ATOM | 677 | CA | LEU | A | 120 | 74.913 | 37.097 | −7.683 | 1.00 | 20.66 | C |
| ATOM | 678 | CB | LEU | A | 120 | 74.609 | 36.193 | −6.483 | 1.00 | 20.51 | C |
| ATOM | 679 | CG | LEU | A | 120 | 75.197 | 34.788 | −6.594 | 1.00 | 21.71 | C |
| ATOM | 680 | CD1 | LEU | A | 120 | 75.218 | 34.103 | −5.236 | 1.00 | 23.12 | C |
| ATOM | 681 | CD2 | LEU | A | 120 | 74.403 | 33.967 | −7.591 | 1.00 | 22.78 | C |
| ATOM | 682 | C | LEU | A | 120 | 74.253 | 38.436 | −7.455 | 1.00 | 20.91 | C |
| ATOM | 683 | O | LEU | A | 120 | 74.853 | 39.319 | −6.848 | 1.00 | 20.71 | O |
| ATOM | 684 | N | GLU | A | 121 | 73.015 | 38.588 | −7.919 | 1.00 | 21.58 | N |
| ATOM | 685 | CA | GLU | A | 121 | 72.238 | 39.775 | −7.581 | 1.00 | 22.82 | C |
| ATOM | 686 | CB | GLU | A | 121 | 70.883 | 39.780 | −8.308 | 1.00 | 23.93 | C |
| ATOM | 687 | CG | GLU | A | 121 | 69.759 | 39.096 | −7.559 | 1.00 | 26.70 | C |
| ATOM | 688 | CD | GLU | A | 121 | 68.493 | 38.950 | −8.387 | 1.00 | 30.59 | C |
| ATOM | 689 | OE1 | GLU | A | 121 | 67.830 | 39.973 | −8.654 | 1.00 | 33.17 | O |
| ATOM | 690 | OE2 | GLU | A | 121 | 68.159 | 37.811 | −8.759 | 1.00 | 31.26 | O |
| ATOM | 691 | C | GLU | A | 121 | 72.062 | 39.836 | −6.065 | 1.00 | 22.52 | C |
| ATOM | 692 | O | GLU | A | 121 | 72.087 | 38.800 | −5.391 | 1.00 | 22.12 | O |
| ATOM | 693 | N | ARG | A | 122 | 71.908 | 41.043 | −5.533 | 1.00 | 22.16 | N |
| ATOM | 694 | CA | ARG | A | 122 | 71.677 | 41.212 | −4.105 | 1.00 | 22.66 | C |
| ATOM | 695 | CB | ARG | A | 122 | 72.977 | 41.620 | −3.390 | 1.00 | 21.96 | C |
| ATOM | 696 | CG | ARG | A | 122 | 72.814 | 41.952 | −1.920 | 1.00 | 21.41 | C |
| ATOM | 697 | CD | ARG | A | 122 | 74.128 | 42.195 | −1.161 | 1.00 | 21.79 | C |
| ATOM | 698 | NE | ARG | A | 122 | 74.932 | 43.293 | −1.726 | 1.00 | 20.54 | N |
| ATOM | 699 | CZ | ARG | A | 122 | 74.781 | 44.581 | −1.418 | 1.00 | 21.80 | C |
| ATOM | 700 | NH1 | ARG | A | 122 | 73.860 | 44.973 | −0.543 | 1.00 | 21.45 | N |
| ATOM | 701 | NH2 | ARG | A | 122 | 75.568 | 45.489 | −1.977 | 1.00 | 23.39 | N |
| ATOM | 702 | C | ARG | A | 122 | 70.575 | 42.249 | −3.864 | 1.00 | 23.50 | C |
| ATOM | 703 | O | ARG | A | 122 | 70.764 | 43.419 | −4.156 | 1.00 | 23.59 | O |
| ATOM | 704 | N | PRO | A | 123 | 69.429 | 41.818 | −3.330 | 1.00 | 24.57 | N |
| ATOM | 705 | CA | PRO | A | 123 | 68.384 | 42.756 | −2.888 | 1.00 | 25.12 | C |
| ATOM | 706 | CB | PRO | A | 123 | 67.233 | 41.832 | −2.448 | 1.00 | 25.10 | C |
| ATOM | 707 | CG | PRO | A | 123 | 67.552 | 40.494 | −3.044 | 1.00 | 25.14 | C |
| ATOM | 708 | CD | PRO | A | 123 | 69.047 | 40.411 | −3.109 | 1.00 | 24.18 | C |
| ATOM | 709 | C | PRO | A | 123 | 68.860 | 43.599 | −1.703 | 1.00 | 25.82 | C |
| ATOM | 710 | O | PRO | A | 123 | 69.678 | 43.129 | −0.900 | 1.00 | 25.56 | O |
| ATOM | 711 | N | GLU | A | 124 | 68.358 | 44.830 | −1.607 | 1.00 | 26.56 | N |
| ATOM | 712 | CA | GLU | A | 124 | 68.738 | 45.750 | −0.533 | 1.00 | 27.26 | C |
| ATOM | 713 | CB | GLU | A | 124 | 69.952 | 46.591 | −0.958 | 1.00 | 27.95 | C |
| ATOM | 714 | CG | GLU | A | 124 | 70.730 | 47.274 | 0.171 | 1.00 | 30.91 | C |
| ATOM | 715 | CD | GLU | A | 124 | 71.867 | 48.140 | −0.367 | 1.00 | 35.36 | C |
| ATOM | 716 | OE1 | GLU | A | 124 | 71.616 | 48.930 | −1.311 | 1.00 | 37.56 | O |
| ATOM | 717 | OE2 | GLU | A | 124 | 73.017 | 48.036 | 0.134 | 1.00 | 36.68 | O |
| ATOM | 718 | C | GLU | A | 124 | 67.544 | 46.649 | −0.201 | 1.00 | 26.82 | C |
| ATOM | 719 | O | GLU | A | 124 | 67.053 | 47.358 | −1.078 | 1.00 | 27.29 | O |
| ATOM | 720 | N | PRO | A | 125 | 67.061 | 46.617 | 1.045 | 1.00 | 25.92 | N |
| ATOM | 721 | CA | PRO | A | 125 | 67.599 | 45.755 | 2.101 | 1.00 | 24.79 | C |
| ATOM | 722 | CB | PRO | A | 125 | 67.062 | 46.403 | 3.373 | 1.00 | 24.95 | C |
| ATOM | 723 | CG | PRO | A | 125 | 65.759 | 46.993 | 2.960 | 1.00 | 24.96 | C |
| ATOM | 724 | CD | PRO | A | 125 | 65.936 | 47.437 | 1.530 | 1.00 | 25.81 | C |
| ATOM | 725 | C | PRO | A | 125 | 67.095 | 44.316 | 1.989 | 1.00 | 23.97 | C |
| ATOM | 726 | O | PRO | A | 125 | 66.109 | 44.040 | 1.287 | 1.00 | 23.35 | O |
| ATOM | 727 | N | VAL | A | 126 | 67.789 | 43.412 | 2.670 | 1.00 | 23.02 | N |
| ATOM | 728 | CA | VAL | A | 126 | 67.507 | 41.987 | 2.583 | 1.00 | 22.32 | C |
| ATOM | 729 | CB | VAL | A | 126 | 68.333 | 41.305 | 1.439 | 1.00 | 22.37 | C |
| ATOM | 730 | CG1 | VAL | A | 126 | 69.815 | 41.129 | 1.837 | 1.00 | 21.96 | C |
| ATOM | 731 | CG2 | VAL | A | 126 | 67.732 | 39.971 | 1.028 | 1.00 | 22.11 | C |
| ATOM | 732 | C | VAL | A | 126 | 67.809 | 41.342 | 3.925 | 1.00 | 22.20 | C |
| ATOM | 733 | O | VAL | A | 126 | 68.600 | 41.866 | 4.723 | 1.00 | 22.19 | O |
| ATOM | 734 | N | GLN | A | 127 | 67.159 | 40.209 | 4.166 | 1.00 | 21.35 | N |
| ATOM | 735 | CA | GLN | A | 127 | 67.429 | 39.378 | 5.323 | 1.00 | 20.84 | C |
| ATOM | 736 | CB | GLN | A | 127 | 66.653 | 39.883 | 6.540 | 1.00 | 20.45 | C |
| ATOM | 737 | CG | GLN | A | 127 | 66.866 | 39.053 | 7.796 | 1.00 | 20.49 | C |
| ATOM | 738 | CD | GLN | A | 127 | 66.156 | 39.632 | 8.999 | 1.00 | 22.00 | C |
| ATOM | 739 | OE1 | GLN | A | 127 | 64.953 | 39.891 | 8.944 | 1.00 | 22.10 | O |
| ATOM | 740 | NE2 | GLN | A | 127 | 66.892 | 39.842 | 10.082 | 1.00 | 20.41 | N |
| ATOM | 741 | C | GLN | A | 127 | 66.996 | 37.952 | 4.963 | 1.00 | 20.56 | C |
| ATOM | 742 | O | GLN | A | 127 | 65.917 | 37.760 | 4.392 | 1.00 | 20.00 | O |
| ATOM | 743 | N | ASP | A | 128 | 67.845 | 36.967 | 5.250 | 1.00 | 19.63 | N |
| ATOM | 744 | CA | ASP | A | 128 | 67.454 | 35.593 | 5.008 | 1.00 | 19.54 | C |
| ATOM | 745 | CB | ASP | A | 128 | 68.672 | 34.650 | 4.844 | 1.00 | 19.72 | C |
| ATOM | 746 | CG | ASP | A | 128 | 69.276 | 34.181 | 6.158 | 1.00 | 21.27 | C |
| ATOM | 747 | OD1 | ASP | A | 128 | 68.578 | 34.093 | 7.189 | 1.00 | 22.90 | O |
| ATOM | 748 | OD2 | ASP | A | 128 | 70.480 | 33.843 | 6.237 | 1.00 | 24.10 | O |
| ATOM | 749 | C | ASP | A | 128 | 66.381 | 35.126 | 6.016 | 1.00 | 19.14 | C |
| ATOM | 750 | O | ASP | A | 128 | 66.220 | 35.724 | 7.079 | 1.00 | 18.98 | O |
| ATOM | 751 | N | LEU | A | 129 | 65.642 | 34.077 | 5.658 | 1.00 | 18.65 | N |
| ATOM | 752 | CA | LEU | A | 129 | 64.485 | 33.651 | 6.429 | 1.00 | 18.28 | C |
| ATOM | 753 | CB | LEU | A | 129 | 63.611 | 32.662 | 5.619 | 1.00 | 17.80 | C |
| ATOM | 754 | CG | LEU | A | 129 | 62.291 | 32.181 | 6.245 | 1.00 | 17.80 | C |
| ATOM | 755 | CD1 | LEU | A | 129 | 61.344 | 33.350 | 6.565 | 1.00 | 16.86 | C |
| ATOM | 756 | CD2 | LEU | A | 129 | 61.591 | 31.180 | 5.327 | 1.00 | 15.45 | C |
| ATOM | 757 | C | LEU | A | 129 | 64.861 | 33.096 | 7.804 | 1.00 | 18.57 | C |
| ATOM | 758 | O | LEU | A | 129 | 64.095 | 33.220 | 8.760 | 1.00 | 18.46 | O |
| ATOM | 759 | N | PHE | A | 130 | 66.047 | 32.503 | 7.908 | 1.00 | 18.90 | N |
| ATOM | 760 | CA | PHE | A | 130 | 66.545 | 32.032 | 9.200 | 1.00 | 19.18 | C |
| ATOM | 761 | CB | PHE | A | 130 | 67.887 | 31.311 | 9.033 | 1.00 | 19.86 | C |
| ATOM | 762 | CG | PHE | A | 130 | 68.531 | 30.931 | 10.339 | 1.00 | 22.10 | C |
| ATOM | 763 | CD1 | PHE | A | 130 | 69.471 | 31.764 | 10.933 | 1.00 | 23.65 | C |
| ATOM | 764 | CE1 | PHE | A | 130 | 70.069 | 31.423 | 12.155 | 1.00 | 26.57 | C |
| ATOM | 765 | CZ | PHE | A | 130 | 69.712 | 30.232 | 12.792 | 1.00 | 25.84 | C |
| ATOM | 766 | CE2 | PHE | A | 130 | 68.765 | 29.398 | 12.206 | 1.00 | 26.84 | C |
| ATOM | 767 | CD2 | PHE | A | 130 | 68.179 | 29.748 | 10.982 | 1.00 | 24.38 | C |
| ATOM | 768 | C | PHE | A | 130 | 66.704 | 33.176 | 10.203 | 1.00 | 19.08 | C |
| ATOM | 769 | O | PHE | A | 130 | 66.287 | 33.060 | 11.374 | 1.00 | 17.75 | O |
| ATOM | 770 | N | ASP | A | 131 | 67.316 | 34.274 | 9.753 | 1.00 | 19.37 | N |
| ATOM | 771 | CA | ASP | A | 131 | 67.489 | 35.442 | 10.622 | 1.00 | 20.03 | C |
| ATOM | 772 | CB | ASP | A | 131 | 68.375 | 36.505 | 9.966 | 1.00 | 20.64 | C |
| ATOM | 773 | CG | ASP | A | 131 | 69.836 | 36.090 | 9.894 | 1.00 | 23.72 | C |
| ATOM | 774 | OD1 | ASP | A | 131 | 70.258 | 35.197 | 10.671 | 1.00 | 28.11 | O |
| ATOM | 775 | OD2 | ASP | A | 131 | 70.642 | 36.603 | 9.084 | 1.00 | 27.01 | O |
| ATOM | 776 | C | ASP | A | 131 | 66.136 | 36.030 | 10.947 | 1.00 | 19.62 | C |
| ATOM | 777 | O | ASP | A | 131 | 65.868 | 36.368 | 12.086 | 1.00 | 19.32 | O |
| ATOM | 778 | N | PHE | A | 132 | 65.275 | 36.133 | 9.936 | 1.00 | 19.50 | N |
| ATOM | 779 | CA | PHE | A | 132 | 63.969 | 36.758 | 10.094 | 1.00 | 19.48 | C |
| ATOM | 780 | CB | PHE | A | 132 | 63.233 | 36.740 | 8.754 | 1.00 | 19.50 | C |
| ATOM | 781 | CG | PHE | A | 132 | 61.939 | 37.481 | 8.749 | 1.00 | 20.08 | C |
| ATOM | 782 | CD1 | PHE | A | 132 | 61.906 | 38.839 | 8.455 | 1.00 | 21.55 | C |
| ATOM | 783 | CE1 | PHE | A | 132 | 60.704 | 39.535 | 8.433 | 1.00 | 22.42 | C |
| ATOM | 784 | CZ | PHE | A | 132 | 59.506 | 38.861 | 8.680 | 1.00 | 22.64 | C |
| ATOM | 785 | CE2 | PHE | A | 132 | 59.522 | 37.505 | 8.962 | 1.00 | 21.42 | C |
| ATOM | 786 | CD2 | PHE | A | 132 | 60.734 | 36.814 | 8.991 | 1.00 | 21.38 | C |
| ATOM | 787 | C | PHE | A | 132 | 63.186 | 36.015 | 11.167 | 1.00 | 20.30 | C |
| ATOM | 788 | O | PHE | A | 132 | 62.643 | 36.642 | 12.088 | 1.00 | 20.08 | O |
| ATOM | 789 | N | ILE | A | 133 | 63.131 | 34.682 | 11.064 | 1.00 | 20.79 | N |
| ATOM | 790 | CA | ILE | A | 133 | 62.411 | 33.875 | 12.064 | 1.00 | 21.52 | C |
| ATOM | 791 | CB | ILE | A | 133 | 62.195 | 32.429 | 11.583 | 1.00 | 21.38 | C |
| ATOM | 792 | CG1 | ILE | A | 133 | 61.215 | 32.402 | 10.402 | 1.00 | 19.84 | C |
| ATOM | 793 | CD1 | ILE | A | 133 | 61.200 | 31.104 | 9.665 | 1.00 | 16.74 | C |
| ATOM | 794 | CG2 | ILE | A | 133 | 61.665 | 31.539 | 12.747 | 1.00 | 21.31 | C |
| ATOM | 795 | C | ILE | A | 133 | 63.097 | 33.868 | 13.438 | 1.00 | 22.84 | C |
| ATOM | 796 | O | ILE | A | 133 | 62.430 | 33.825 | 14.472 | 1.00 | 22.75 | O |
| ATOM | 797 | N | THR | A | 134 | 64.423 | 33.888 | 13.446 | 1.00 | 23.77 | N |
| ATOM | 798 | CA | THR | A | 134 | 65.167 | 34.000 | 14.696 | 1.00 | 25.24 | C |
| ATOM | 799 | CB | THR | A | 134 | 66.683 | 33.972 | 14.427 | 1.00 | 24.97 | C |
| ATOM | 800 | OG1 | THR | A | 134 | 67.056 | 32.682 | 13.921 | 1.00 | 24.99 | O |
| ATOM | 801 | CG2 | THR | A | 134 | 67.486 | 34.101 | 15.735 | 1.00 | 25.41 | C |
| ATOM | 802 | C | THR | A | 134 | 64.779 | 35.286 | 15.433 | 1.00 | 26.05 | C |
| ATOM | 803 | O | THR | A | 134 | 64.514 | 35.271 | 16.636 | 1.00 | 26.27 | O |
| ATOM | 804 | N | GLU | A | 135 | 64.728 | 36.386 | 14.693 | 1.00 | 27.17 | N |
| ATOM | 805 | CA | GLU | A | 135 | 64.424 | 37.693 | 15.268 | 1.00 | 28.48 | C |
| ATOM | 806 | CB | GLU | A | 135 | 64.830 | 38.807 | 14.302 | 1.00 | 28.91 | C |
| ATOM | 807 | CG | GLU | A | 135 | 66.282 | 39.221 | 14.449 | 1.00 | 32.87 | C |
| ATOM | 808 | CD | GLU | A | 135 | 66.702 | 40.288 | 13.450 | 1.00 | 37.37 | C |
| ATOM | 809 | OE1 | GLU | A | 135 | 65.813 | 41.022 | 12.939 | 1.00 | 38.58 | O |
| ATOM | 810 | OE2 | GLU | A | 135 | 67.927 | 40.383 | 13.177 | 1.00 | 38.32 | O |
| ATOM | 811 | C | GLU | A | 135 | 62.958 | 37.853 | 15.657 | 1.00 | 28.36 | C |
| ATOM | 812 | O | GLU | A | 135 | 62.656 | 38.416 | 16.710 | 1.00 | 27.93 | O |
| ATOM | 813 | N | ARG | A | 136 | 62.056 | 37.345 | 14.817 | 1.00 | 27.83 | N |
| ATOM | 814 | CA | ARG | A | 136 | 60.635 | 37.631 | 14.983 | 1.00 | 27.91 | C |
| ATOM | 815 | CB | ARG | A | 136 | 60.022 | 38.109 | 13.657 | 1.00 | 28.19 | C |
| ATOM | 816 | CG | ARG | A | 136 | 60.551 | 39.487 | 13.244 | 1.00 | 30.84 | C |
| ATOM | 817 | CD | ARG | A | 136 | 60.046 | 40.034 | 11.909 | 1.00 | 33.40 | C |
| ATOM | 818 | NE | ARG | A | 136 | 58.583 | 40.081 | 11.805 | 1.00 | 35.56 | N |
| ATOM | 819 | CZ | ARG | A | 136 | 57.907 | 40.978 | 11.081 | 1.00 | 35.76 | C |
| ATOM | 820 | NH1 | ARG | A | 136 | 58.556 | 41.923 | 10.403 | 1.00 | 35.79 | N |
| ATOM | 821 | NH2 | ARG | A | 136 | 56.580 | 40.938 | 11.041 | 1.00 | 34.21 | N |
| ATOM | 822 | C | ARG | A | 136 | 59.836 | 36.488 | 15.594 | 1.00 | 26.86 | C |
| ATOM | 823 | O | ARG | A | 136 | 58.683 | 36.677 | 15.980 | 1.00 | 27.36 | O |
| ATOM | 824 | N | GLY | A | 137 | 60.452 | 35.316 | 15.713 | 1.00 | 25.61 | N |
| ATOM | 825 | CA | GLY | A | 137 | 59.754 | 34.134 | 16.187 | 1.00 | 24.72 | C |
| ATOM | 826 | C | GLY | A | 137 | 58.763 | 33.584 | 15.156 | 1.00 | 24.39 | C |
| ATOM | 827 | O | GLY | A | 137 | 58.796 | 33.952 | 13.969 | 1.00 | 23.90 | O |
| ATOM | 828 | N | ALA | A | 138 | 57.880 | 32.699 | 15.615 | 1.00 | 23.04 | N |
| ATOM | 829 | CA | ALA | A | 138 | 56.864 | 32.089 | 14.760 | 1.00 | 22.25 | C |
| ATOM | 830 | CB | ALA | A | 138 | 55.895 | 31.269 | 15.612 | 1.00 | 22.29 | C |
| ATOM | 831 | C | ALA | A | 138 | 56.101 | 33.152 | 13.968 | 1.00 | 22.02 | C |
| ATOM | 832 | O | ALA | A | 138 | 55.694 | 34.174 | 14.523 | 1.00 | 21.11 | O |
| ATOM | 833 | N | LEU | A | 139 | 55.914 | 32.906 | 12.671 | 1.00 | 20.97 | N |
| ATOM | 834 | CA | LEU | A | 139 | 55.223 | 33.860 | 11.814 | 1.00 | 20.45 | C |
| ATOM | 835 | CB | LEU | A | 139 | 55.673 | 33.676 | 10.358 | 1.00 | 19.75 | C |
| ATOM | 836 | CG | LEU | A | 139 | 57.194 | 33.668 | 10.121 | 1.00 | 19.78 | C |
| ATOM | 837 | CD1 | LEU | A | 139 | 57.509 | 33.578 | 8.624 | 1.00 | 17.80 | C |
| ATOM | 838 | CD2 | LEU | A | 139 | 57.871 | 34.908 | 10.772 | 1.00 | 19.37 | C |
| ATOM | 839 | C | LEU | A | 139 | 53.706 | 33.707 | 11.938 | 1.00 | 20.32 | C |
| ATOM | 840 | O | LEU | A | 139 | 53.209 | 32.589 | 12.007 | 1.00 | 20.36 | O |
| ATOM | 841 | N | GLN | A | 140 | 52.979 | 34.828 | 11.950 | 1.00 | 19.81 | N |
| ATOM | 842 | CA | GLN | A | 140 | 51.530 | 34.796 | 11.751 | 1.00 | 19.77 | C |
| ATOM | 843 | CB | GLN | A | 140 | 50.958 | 36.210 | 11.624 | 1.00 | 20.35 | C |
| ATOM | 844 | CG | GLN | A | 140 | 50.938 | 37.006 | 12.913 | 1.00 | 24.46 | C |
| ATOM | 845 | CD | GLN | A | 140 | 50.666 | 38.484 | 12.674 | 1.00 | 30.48 | C |
| ATOM | 846 | OE1 | GLN | A | 140 | 49.836 | 38.846 | 11.827 | 1.00 | 32.68 | O |
| ATOM | 847 | NE2 | GLN | A | 140 | 51.357 | 39.343 | 13.421 | 1.00 | 32.67 | N |
| ATOM | 848 | C | GLN | A | 140 | 51.211 | 34.035 | 10.469 | 1.00 | 18.78 | C |
| ATOM | 849 | O | GLU | A | 140 | 51.957 | 34.118 | 9.494 | 1.00 | 17.03 | O |
| ATOM | 850 | N | GLU | A | 141 | 50.088 | 33.325 | 10.453 | 1.00 | 18.57 | N |
| ATOM | 851 | CA | GLU | A | 141 | 49.769 | 32.482 | 9.296 | 1.00 | 18.81 | C |
| ATOM | 852 | CB | GLU | A | 141 | 48.563 | 31.588 | 9.579 | 1.00 | 18.86 | C |
| ATOM | 853 | CG | GLU | A | 141 | 48.922 | 30.461 | 10.531 | 1.00 | 20.50 | C |
| ATOM | 854 | CD | GLU | A | 141 | 47.785 | 29.504 | 10.762 | 1.00 | 20.11 | C |
| ATOM | 855 | OE1 | GLU | A | 141 | 47.107 | 29.151 | 9.779 | 1.00 | 20.79 | O |
| ATOM | 856 | OE2 | GLU | A | 141 | 47.572 | 29.120 | 11.933 | 1.00 | 21.96 | O |
| ATOM | 857 | C | GLU | A | 141 | 49.605 | 33.235 | 7.970 | 1.00 | 18.75 | C |
| ATOM | 858 | O | GLU | A | 141 | 49.969 | 32.702 | 6.931 | 1.00 | 18.07 | O |
| ATOM | 859 | N | GLU | A | 142 | 49.048 | 34.454 | 8.002 | 1.00 | 18.37 | N |
| ATOM | 860 | CA | GLU | A | 142 | 48.939 | 35.263 | 6.787 | 1.00 | 18.46 | C |
| ATOM | 861 | CB | GLU | A | 142 | 48.216 | 36.589 | 7.078 | 1.00 | 18.72 | C |
| ATOM | 862 | CG | GLU | A | 142 | 48.076 | 37.515 | 5.889 | 1.00 | 20.48 | C |
| ATOM | 863 | CD | GLU | A | 142 | 47.411 | 38.836 | 6.241 | 1.00 | 24.11 | C |
| ATOM | 864 | OE1 | GLU | A | 142 | 48.061 | 39.692 | 6.891 | 1.00 | 23.55 | O |
| ATOM | 865 | OE2 | GLU | A | 142 | 46.232 | 39.017 | 5.851 | 1.00 | 25.93 | O |
| ATOM | 866 | C | GLU | A | 142 | 50.329 | 35.529 | 6.179 | 1.00 | 17.83 | C |
| ATOM | 867 | O | GLU | A | 142 | 50.506 | 35.530 | 4.959 | 1.00 | 17.80 | O |
| ATOM | 868 | N | LEU | A | 143 | 51.309 | 35.761 | 7.037 | 1.00 | 16.88 | N |
| ATOM | 869 | CA | LEU | A | 143 | 52.653 | 36.029 | 6.568 | 1.00 | 16.41 | C |
| ATOM | 870 | CB | LEU | A | 143 | 53.497 | 36.667 | 7.666 | 1.00 | 16.81 | C |
| ATOM | 871 | CG | LEU | A | 143 | 54.952 | 36.998 | 7.288 | 1.00 | 16.83 | C |
| ATOM | 872 | CD1 | LEU | A | 143 | 54.999 | 37.909 | 6.049 | 1.00 | 15.94 | C |
| ATOM | 873 | CD2 | LEU | A | 143 | 55.625 | 37.670 | 8.464 | 1.00 | 16.70 | C |
| ATOM | 874 | C | LEU | A | 143 | 53.307 | 34.749 | 6.057 | 1.00 | 15.72 | C |
| ATOM | 875 | O | LEU | A | 143 | 53.921 | 34.745 | 4.983 | 1.00 | 15.44 | O |
| ATOM | 876 | N | ALA | A | 144 | 53.173 | 33.670 | 6.824 | 1.00 | 14.81 | N |
| ATOM | 877 | CA | ALA | A | 144 | 53.692 | 32.364 | 6.404 | 1.00 | 14.61 | C |
| ATOM | 878 | CB | ALA | A | 144 | 53.444 | 31.327 | 7.470 | 1.00 | 14.33 | C |
| ATOM | 879 | C | ALA | A | 144 | 53.078 | 31.914 | 5.077 | 1.00 | 14.82 | C |
| ATOM | 880 | O | ALA | A | 144 | 53.754 | 31.270 | 4.253 | 1.00 | 14.19 | O |
| ATOM | 881 | N | ARG | A | 145 | 51.796 | 32.235 | 4.884 | 1.00 | 14.19 | N |
| ATOM | 882 | CA | ARG | A | 145 | 51.090 | 31.869 | 3.666 | 1.00 | 15.16 | C |
| ATOM | 883 | CB | ARG | A | 145 | 49.587 | 32.205 | 3.764 | 1.00 | 15.23 | C |
| ATOM | 884 | CG | ARG | A | 145 | 48.803 | 32.031 | 2.453 | 1.00 | 16.28 | C |
| ATOM | 885 | CD | ARG | A | 145 | 47.303 | 32.380 | 2.564 | 1.00 | 18.20 | C |
| ATOM | 886 | NE | ARG | A | 145 | 46.693 | 31.573 | 3.615 | 1.00 | 17.28 | N |
| ATOM | 887 | CZ | ARG | A | 145 | 46.238 | 32.049 | 4.761 | 1.00 | 17.72 | C |
| ATOM | 888 | NH1 | ARG | A | 145 | 46.270 | 33.352 | 5.018 | 1.00 | 17.11 | N |
| ATOM | 889 | NH2 | ARG | A | 145 | 45.747 | 31.212 | 5.657 | 1.00 | 18.63 | N |
| ATOM | 890 | C | ARG | A | 145 | 51.727 | 32.562 | 2.471 | 1.00 | 15.43 | C |
| ATOM | 891 | O | ARG | A | 145 | 52.034 | 31.917 | 1.470 | 1.00 | 16.61 | O |
| ATOM | 892 | N | SER | A | 146 | 51.941 | 33.868 | 2.578 | 1.00 | 15.08 | N |
| ATOM | 893 | CA | SER | A | 146 | 52.557 | 34.618 | 1.491 | 1.00 | 15.89 | C |
| ATOM | 894 | CB | SER | A | 146 | 52.558 | 36.114 | 1.823 | 1.00 | 15.77 | C |
| ATOM | 895 | OG | SER | A | 146 | 53.374 | 36.817 | 0.907 | 1.00 | 18.17 | O |
| ATOM | 896 | C | SER | A | 146 | 53.976 | 34.104 | 1.170 | 1.00 | 15.75 | C |
| ATOM | 897 | O | SER | A | 146 | 54.311 | 33.849 | 0.000 | 1.00 | 15.69 | O |
| ATOM | 898 | N | PHE | A | 147 | 54.777 | 33.926 | 2.220 | 1.00 | 15.20 | N |
| ATOM | 899 | CA | PHE | A | 147 | 56.145 | 33.423 | 2.104 | 1.00 | 15.40 | C |
| ATOM | 900 | CB | PHE | A | 147 | 56.801 | 33.392 | 3.487 | 1.00 | 15.25 | C |
| ATOM | 901 | CG | PHE | A | 147 | 57.345 | 34.724 | 3.939 | 1.00 | 16.31 | C |
| ATOM | 902 | CD1 | PHE | A | 147 | 57.041 | 35.903 | 3.246 | 1.00 | 17.31 | C |
| ATOM | 903 | CE1 | PHE | A | 147 | 57.552 | 37.121 | 3.663 | 1.00 | 18.81 | C |
| ATOM | 904 | CZ | PHE | A | 147 | 58.389 | 37.178 | 4.790 | 1.00 | 18.13 | C |
| ATOM | 905 | CE2 | PHE | A | 147 | 58.696 | 36.017 | 5.480 | 1.00 | 17.81 | C |
| ATOM | 906 | CD2 | PHE | A | 147 | 58.176 | 34.793 | 5.052 | 1.00 | 16.34 | C |
| ATOM | 907 | C | PHE | A | 147 | 56.195 | 32.024 | 1.483 | 1.00 | 14.94 | C |
| ATOM | 908 | O | PHE | A | 147 | 56.927 | 31.786 | 0.522 | 1.00 | 15.80 | O |
| ATOM | 909 | N | PHE | A | 148 | 55.407 | 31.114 | 2.033 | 1.00 | 14.80 | N |
| ATOM | 910 | CA | PHE | A | 148 | 55.354 | 29.733 | 1.549 | 1.00 | 15.37 | C |
| ATOM | 911 | CB | PHE | A | 148 | 54.409 | 28.887 | 2.418 | 1.00 | 14.71 | C |
| ATOM | 912 | CG | PHE | A | 148 | 54.574 | 27.399 | 2.224 | 1.00 | 14.42 | C |
| ATOM | 913 | CD1 | PHE | A | 148 | 55.810 | 26.776 | 2.456 | 1.00 | 13.47 | C |
| ATOM | 914 | CE1 | PHE | A | 148 | 55.962 | 25.379 | 2.277 | 1.00 | 10.13 | C |
| ATOM | 915 | CZ | PHE | A | 148 | 54.876 | 24.618 | 1.864 | 1.00 | 12.94 | C |
| ATOM | 916 | CE2 | PHE | A | 148 | 53.635 | 25.237 | 1.625 | 1.00 | 14.02 | C |
| ATOM | 917 | CD2 | PHE | A | 148 | 53.495 | 26.622 | 1.813 | 1.00 | 14.27 | C |
| ATOM | 918 | C | PHE | A | 148 | 54.898 | 29.648 | 0.089 | 1.00 | 15.20 | C |
| ATOM | 919 | O | PHE | A | 148 | 55.459 | 28.902 | −0.703 | 1.00 | 14.97 | O |
| ATOM | 920 | N | TRP | A | 149 | 53.866 | 30.413 | −0.253 | 1.00 | 15.54 | N |
| ATOM | 921 | CA | TRP | A | 149 | 53.393 | 30.477 | −1.634 | 1.00 | 15.37 | C |
| ATOM | 922 | CB | TRP | A | 149 | 52.230 | 31.470 | −1.739 | 1.00 | 15.34 | C |
| ATOM | 923 | CG | TRP | A | 149 | 51.671 | 31.606 | −3.110 | 1.00 | 15.24 | C |
| ATOM | 924 | CD1 | TRP | A | 149 | 52.070 | 32.494 | −4.075 | 1.00 | 14.51 | C |
| ATOM | 925 | NE1 | TRP | A | 149 | 51.301 | 32.333 | −5.205 | 1.00 | 15.75 | N |
| ATOM | 926 | CE2 | TRP | A | 149 | 50.394 | 31.326 | −4.998 | 1.00 | 15.41 | C |
| ATOM | 927 | CD2 | TRP | A | 149 | 50.595 | 30.845 | −3.682 | 1.00 | 15.51 | C |
| ATOM | 928 | CE3 | TRP | A | 149 | 49.766 | 29.804 | −3.210 | 1.00 | 15.20 | C |
| ATOM | 929 | CZ3 | TRP | A | 149 | 48.777 | 29.287 | −4.064 | 1.00 | 14.40 | C |
| ATOM | 930 | CH2 | TRP | A | 149 | 48.614 | 29.788 | −5.376 | 1.00 | 15.19 | C |
| ATOM | 931 | CZ2 | TRP | A | 149 | 49.405 | 30.804 | −5.857 | 1.00 | 15.74 | C |
| ATOM | 932 | C | TRP | A | 149 | 54.516 | 30.881 | −2.585 | 1.00 | 15.47 | C |
| ATOM | 933 | O | TRP | A | 149 | 54.709 | 30.266 | −3.637 | 1.00 | 15.67 | O |
| ATOM | 934 | N | GLN | A | 150 | 55.267 | 31.913 | −2.213 | 1.00 | 16.07 | N |
| ATOM | 935 | CA | GLN | A | 150 | 56.354 | 32.394 | −3.063 | 1.00 | 16.16 | C |
| ATOM | 936 | CB | GLN | A | 150 | 56.926 | 33.704 | −2.522 | 1.00 | 16.54 | C |
| ATOM | 937 | CG | GLN | A | 150 | 56.012 | 34.904 | −2.760 | 1.00 | 17.72 | C |
| ATOM | 938 | CD | GLN | A | 150 | 56.654 | 36.188 | −2.309 | 1.00 | 20.82 | C |
| ATOM | 939 | OE1 | GLN | A | 150 | 57.668 | 36.594 | −2.860 | 1.00 | 20.46 | O |
| ATOM | 940 | NE2 | GLN | A | 150 | 56.078 | 36.825 | −1.291 | 1.00 | 22.67 | N |
| ATOM | 941 | C | GLN | A | 150 | 57.470 | 31.366 | −3.231 | 1.00 | 16.22 | C |
| ATOM | 942 | O | GLN | A | 150 | 58.068 | 31.271 | −4.311 | 1.00 | 16.09 | O |
| ATOM | 943 | N | VAL | A | 151 | 57.747 | 30.613 | −2.165 | 1.00 | 15.69 | N |
| ATOM | 944 | CA | VAL | A | 151 | 58.719 | 29.528 | −2.219 | 1.00 | 15.67 | C |
| ATOM | 945 | CB | VAL | A | 151 | 58.973 | 28.914 | −0.819 | 1.00 | 15.75 | C |
| ATOM | 946 | CG1 | VAL | A | 151 | 59.838 | 27.661 | −0.920 | 1.00 | 15.14 | C |
| ATOM | 947 | CG2 | VAL | A | 151 | 59.648 | 29.950 | 0.087 | 1.00 | 14.26 | C |
| ATOM | 948 | C | VAL | A | 151 | 58.232 | 28.454 | −3.186 | 1.00 | 15.73 | C |
| ATOM | 949 | O | VAL | A | 151 | 58.979 | 28.003 | −4.048 | 1.00 | 15.25 | O |
| ATOM | 950 | N | LEU | A | 152 | 56.967 | 28.065 | −3.046 | 1.00 | 16.29 | N |
| ATOM | 951 | CA | LEU | A | 152 | 56.348 | 27.138 | −3.992 | 1.00 | 17.18 | C |
| ATOM | 952 | CB | LEU | A | 152 | 54.859 | 26.947 | −3.658 | 1.00 | 17.37 | C |
| ATOM | 953 | CG | LEU | A | 152 | 54.467 | 25.690 | −2.874 | 1.00 | 19.91 | C |
| ATOM | 954 | CD1 | LEU | A | 152 | 54.643 | 24.445 | −3.756 | 1.00 | 22.90 | C |
| ATOM | 955 | CD2 | LEU | A | 152 | 55.236 | 25.494 | −1.621 | 1.00 | 23.13 | C |
| ATOM | 956 | C | LEU | A | 152 | 56.512 | 27.572 | −5.453 | 1.00 | 16.62 | C |
| ATOM | 957 | O | LEU | A | 152 | 56.889 | 26.765 | −6.299 | 1.00 | 16.65 | O |
| ATOM | 958 | N | GLU | A | 153 | 56.217 | 28.841 | −5.739 | 1.00 | 16.61 | N |
| ATOM | 959 | CA | GLU | A | 153 | 56.333 | 29.375 | −7.096 | 1.00 | 16.62 | C |
| ATOM | 960 | CB | GLU | A | 153 | 55.832 | 30.827 | −7.180 | 1.00 | 16.56 | C |
| ATOM | 961 | CG | GLU | A | 153 | 54.331 | 30.997 | −6.968 | 1.00 | 17.23 | C |
| ATOM | 962 | CD | GLU | A | 153 | 53.514 | 30.514 | −8.156 | 1.00 | 17.86 | C |
| ATOM | 963 | OE1 | GLU | A | 153 | 53.901 | 30.807 | −9.303 | 1.00 | 20.00 | O |
| ATOM | 964 | OE2 | GLU | A | 153 | 52.487 | 29.843 | −7.945 | 1.00 | 17.52 | O |
| ATOM | 965 | C | GLU | A | 153 | 57.777 | 29.297 | −7.568 | 1.00 | 16.68 | C |
| ATOM | 966 | O | GLU | A | 153 | 58.038 | 28.986 | −8.732 | 1.00 | 16.20 | O |
| ATOM | 967 | N | ALA | A | 154 | 58.712 | 29.559 | −6.656 | 1.00 | 16.55 | N |
| ATOM | 968 | CA | ALA | A | 154 | 60.140 | 29.496 | −6.992 | 1.00 | 16.97 | C |
| ATOM | 969 | CB | ALA | A | 154 | 61.004 | 30.188 | −5.919 | 1.00 | 15.90 | C |
| ATOM | 970 | C | ALA | A | 154 | 60.621 | 28.063 | −7.243 | 1.00 | 16.84 | C |
| ATOM | 971 | O | ALA | A | 154 | 61.345 | 27.818 | −8.207 | 1.00 | 17.76 | O |
| ATOM | 972 | N | VAL | A | 155 | 60.218 | 27.126 | −6.386 | 1.00 | 16.64 | N |
| ATOM | 973 | CA | VAL | A | 155 | 60.584 | 25.724 | −6.564 | 1.00 | 16.78 | C |
| ATOM | 974 | CB | VAL | A | 155 | 60.201 | 24.871 | −5.326 | 1.00 | 17.27 | C |
| ATOM | 975 | CG1 | VAL | A | 155 | 60.395 | 23.386 | −5.589 | 1.00 | 16.81 | C |
| ATOM | 976 | CG2 | VAL | A | 155 | 61.032 | 25.313 | −4.084 | 1.00 | 17.68 | C |
| ATOM | 977 | C | VAL | A | 155 | 59.958 | 25.163 | −7.852 | 1.00 | 16.84 | C |
| ATOM | 978 | O | VAL | A | 155 | 60.621 | 24.445 | −8.603 | 1.00 | 16.56 | O |
| ATOM | 979 | N | ARG | A | 156 | 58.690 | 25.491 | −8.107 | 1.00 | 16.70 | N |
| ATOM | 980 | CA | ARG | A | 156 | 58.051 | 25.086 | −9.374 | 1.00 | 17.03 | C |
| ATOM | 981 | CB | ARG | A | 156 | 56.603 | 25.570 | −9.461 | 1.00 | 16.46 | C |
| ATOM | 982 | CG | ARG | A | 156 | 55.645 | 24.827 | −8.564 | 1.00 | 17.08 | C |
| ATOM | 983 | CD | ARG | A | 156 | 54.201 | 25.302 | −8.681 | 1.00 | 16.07 | C |
| ATOM | 984 | NE | ARG | A | 156 | 53.815 | 25.379 | −10.087 | 1.00 | 15.86 | N |
| ATOM | 985 | CZ | ARG | A | 156 | 52.921 | 26.218 | −10.591 | 1.00 | 16.05 | C |
| ATOM | 986 | NH1 | ARG | A | 156 | 52.280 | 27.071 | −9.805 | 1.00 | 14.03 | N |
| ATOM | 987 | NH2 | ARG | A | 156 | 52.672 | 26.199 | −11.895 | 1.00 | 15.89 | N |
| ATOM | 988 | C | ARG | A | 156 | 58.839 | 25.599 | −10.573 | 1.00 | 17.05 | C |
| ATOM | 989 | O | ARG | A | 156 | 59.071 | 24.864 | −11.529 | 1.00 | 17.34 | O |
| ATOM | 990 | N | HIS | A | 157 | 59.266 | 26.855 | −10.522 | 1.00 | 17.35 | N |
| ATOM | 991 | CA | HIS | A | 157 | 60.090 | 27.397 | −11.594 | 1.00 | 18.31 | C |
| ATOM | 992 | CB | HIS | A | 157 | 60.449 | 28.859 | −11.330 | 1.00 | 18.48 | C |
| ATOM | 993 | CG | HIS | A | 157 | 61.374 | 29.443 | −12.351 | 1.00 | 20.28 | C |
| ATOM | 994 | ND1 | HIS | A | 157 | 62.696 | 29.733 | −12.078 | 1.00 | 23.93 | N |
| ATOM | 995 | CE1 | HIS | A | 157 | 63.262 | 30.241 | −13.158 | 1.00 | 23.06 | C |
| ATOM | 996 | NE2 | HIS | A | 157 | 62.356 | 30.288 | −14.118 | 1.00 | 23.55 | N |
| ATOM | 997 | CD2 | HIS | A | 157 | 61.168 | 29.796 | −13.639 | 1.00 | 20.83 | C |
| ATOM | 998 | C | HIS | A | 157 | 61.361 | 26.559 | −11.806 | 1.00 | 18.61 | C |
| ATOM | 999 | O | HIS | A | 157 | 61.691 | 26.199 | −12.947 | 1.00 | 17.98 | O |
| ATOM | 1000 | N | CYS | A | 158 | 62.064 | 26.247 | −10.715 | 1.00 | 18.74 | N |
| ATOM | 1001 | CA | CYS | A | 158 | 63.259 | 25.405 | −10.800 | 1.00 | 19.66 | C |
| ATOM | 1002 | CB | CYS | A | 158 | 63.837 | 25.146 | −9.413 | 1.00 | 19.79 | C |
| ATOM | 1003 | SG | CYS | A | 158 | 64.537 | 26.620 | −8.683 | 1.00 | 22.60 | S |
| ATOM | 1004 | C | CYS | A | 158 | 62.979 | 24.077 | −11.501 | 1.00 | 19.92 | C |
| ATOM | 1005 | O | CYS | A | 158 | 63.677 | 23.712 | −12.447 | 1.00 | 20.10 | O |
| ATOM | 1006 | N | HIS | A | 159 | 61.955 | 23.365 | −11.032 | 1.00 | 20.09 | N |
| ATOM | 1007 | CA | HIS | A | 159 | 61.580 | 22.085 | −11.612 | 1.00 | 20.50 | C |
| ATOM | 1008 | CB | HIS | A | 159 | 60.484 | 21.410 | −10.782 | 1.00 | 20.39 | C |
| ATOM | 1009 | CG | HIS | A | 159 | 60.934 | 20.995 | −9.414 | 1.00 | 21.38 | C |
| ATOM | 1010 | ND1 | HIS | A | 159 | 60.503 | 19.834 | −8.814 | 1.00 | 22.95 | N |
| ATOM | 1011 | CE1 | HIS | A | 159 | 61.055 | 19.727 | −7.616 | 1.00 | 22.06 | C |
| ATOM | 1012 | NE2 | HIS | A | 159 | 61.845 | 20.769 | −7.426 | 1.00 | 21.41 | N |
| ATOM | 1013 | CD2 | HIS | A | 159 | 61.790 | 21.577 | −8.534 | 1.00 | 21.73 | C |
| ATOM | 1014 | C | HIS | A | 159 | 61.175 | 22.194 | −13.092 | 1.00 | 20.62 | C |
| ATOM | 1015 | O | HIS | A | 159 | 61.558 | 21.340 | −13.883 | 1.00 | 20.59 | O |
| ATOM | 1016 | N | ASN | A | 160 | 60.433 | 23.240 | −13.463 | 1.00 | 20.97 | N |
| ATOM | 1017 | CA | ASN | A | 160 | 60.110 | 23.508 | −14.882 | 1.00 | 21.47 | C |
| ATOM | 1018 | CB | ASN | A | 160 | 59.230 | 24.754 | −15.042 | 1.00 | 21.81 | C |
| ATOM | 1019 | CG | AASN | A | 160 | 57.985 | 24.688 | −14.251 | 0.50 | 22.71 | C |
| ATOM | 1020 | CG | BASN | A | 160 | 58.366 | 24.731 | −16.318 | 0.50 | 21.42 | C |
| ATOM | 1021 | OD1 | AASN | A | 160 | 57.565 | 25.688 | −13.683 | 0.50 | 25.62 | O |
| ATOM | 1022 | OD1 | BASN | A | 160 | 58.380 | 25.680 | −17.103 | 0.50 | 21.10 | O |
| ATOM | 1023 | ND2 | AASN | A | 160 | 57.364 | 23.518 | −14.203 | 0.50 | 26.04 | N |
| ATOM | 1024 | ND2 | BASN | A | 160 | 57.598 | 23.664 | −16.506 | 0.50 | 19.99 | N |
| ATOM | 1025 | C | ASN | A | 160 | 61.353 | 23.728 | −15.731 | 1.00 | 21.48 | C |
| ATOM | 1026 | O | ASN | A | 160 | 61.344 | 23.430 | −16.925 | 1.00 | 21.00 | O |
| ATOM | 1027 | N | CYS | A | 161 | 62.404 | 24.278 | −15.115 | 1.00 | 20.77 | N |
| ATOM | 1028 | CA | CYS | A | 161 | 63.691 | 24.462 | −15.773 | 1.00 | 21.07 | C |
| ATOM | 1029 | CB | CYS | A | 161 | 64.395 | 25.716 | −15.231 | 1.00 | 20.76 | C |
| ATOM | 1030 | SG | CYS | A | 161 | 63.499 | 27.235 | −15.609 | 1.00 | 26.51 | S |
| ATOM | 1031 | C | CYS | A | 161 | 64.628 | 23.242 | −15.665 | 1.00 | 20.01 | C |
| ATOM | 1032 | O | CYS | A | 161 | 65.791 | 23.329 | −16.052 | 1.00 | 19.64 | O |
| ATOM | 1033 | N | GLY | A | 162 | 64.141 | 22.124 | −15.130 | 1.00 | 18.99 | N |
| ATOM | 1034 | CA | GLY | A | 162 | 64.965 | 20.921 | −15.005 | 1.00 | 18.24 | C |
| ATOM | 1035 | C | GLY | A | 162 | 65.968 | 20.898 | −13.850 | 1.00 | 17.99 | C |
| ATOM | 1036 | O | GLY | A | 162 | 66.963 | 20.153 | −13.878 | 1.00 | 16.70 | O |
| ATOM | 1037 | N | VAL | A | 163 | 65.696 | 21.678 | −12.805 | 1.00 | 17.86 | N |
| ATOM | 1038 | CA | VAL | A | 163 | 66.634 | 21.799 | −11.688 | 1.00 | 17.74 | C |
| ATOM | 1039 | CB | VAL | A | 163 | 67.173 | 23.251 | −11.564 | 1.00 | 18.57 | C |
| ATOM | 1040 | CG1 | VAL | A | 163 | 67.897 | 23.479 | −10.215 | 1.00 | 17.79 | C |
| ATOM | 1041 | CG2 | VAL | A | 163 | 68.078 | 23.608 | −12.766 | 1.00 | 17.89 | C |
| ATOM | 1042 | C | VAL | A | 163 | 65.970 | 21.373 | −10.374 | 1.00 | 17.54 | C |
| ATOM | 1043 | O | VAL | A | 163 | 64.851 | 21.780 | −10.071 | 1.00 | 17.67 | O |
| ATOM | 1044 | N | LEU | A | 164 | 66.673 | 20.550 | −9.612 | 1.00 | 17.14 | N |
| ATOM | 1045 | CA | LEU | A | 164 | 66.235 | 20.142 | −8.288 | 1.00 | 17.15 | C |
| ATOM | 1046 | CB | LEU | A | 164 | 66.298 | 18.614 | −8.170 | 1.00 | 17.23 | C |
| ATOM | 1047 | CG | LEU | A | 164 | 65.715 | 17.973 | −6.909 | 1.00 | 18.35 | C |
| ATOM | 1048 | CD1 | LEU | A | 164 | 64.183 | 18.038 | −6.939 | 1.00 | 18.69 | C |
| ATOM | 1049 | CD2 | LEU | A | 164 | 66.201 | 16.530 | −6.783 | 1.00 | 15.82 | C |
| ATOM | 1050 | C | LEU | A | 164 | 67.151 | 20.802 | −7.269 | 1.00 | 16.92 | C |
| ATOM | 1051 | O | LEU | A | 164 | 68.367 | 20.594 | −7.305 | 1.00 | 17.02 | O |
| ATOM | 1052 | N | HIS | A | 165 | 66.574 | 21.583 | −6.359 | 1.00 | 16.61 | N |
| ATOM | 1053 | CA | HIS | A | 165 | 67.356 | 22.378 | −5.410 | 1.00 | 15.80 | C |
| ATOM | 1054 | CB | HIS | A | 165 | 66.462 | 23.440 | −4.747 | 1.00 | 16.02 | C |
| ATOM | 1055 | CG | HIS | A | 165 | 67.212 | 24.444 | −3.924 | 1.00 | 15.04 | C |
| ATOM | 1056 | ND1 | HIS | A | 165 | 67.698 | 24.155 | −2.668 | 1.00 | 13.63 | N |
| ATOM | 1057 | CE1 | HIS | A | 165 | 68.311 | 25.218 | −2.175 | 1.00 | 14.79 | C |
| ATOM | 1058 | NE2 | HIS | A | 165 | 68.248 | 26.188 | −3.071 | 1.00 | 16.20 | N |
| ATOM | 1059 | CD2 | HIS | A | 165 | 67.571 | 25.726 | −4.182 | 1.00 | 14.88 | C |
| ATOM | 1060 | C | HIS | A | 165 | 68.065 | 21.520 | −4.352 | 1.00 | 16.12 | C |
| ATOM | 1061 | O | HIS | A | 165 | 69.281 | 21.692 | −4.112 | 1.00 | 15.47 | O |
| ATOM | 1062 | N | ARG | A | 166 | 67.301 | 20.628 | −3.708 | 1.00 | 15.81 | N |
| ATOM | 1063 | CA | ARG | A | 166 | 67.802 | 19.692 | −2.685 | 1.00 | 16.34 | C |
| ATOM | 1064 | CB | ARG | A | 166 | 68.933 | 18.830 | −3.231 | 1.00 | 16.34 | C |
| ATOM | 1065 | CG | ARG | A | 166 | 68.542 | 17.800 | −4.282 | 1.00 | 17.58 | C |
| ATOM | 1066 | CD | ARG | A | 166 | 69.743 | 17.471 | −5.131 | 1.00 | 23.63 | C |
| ATOM | 1067 | NE | ARG | A | 166 | 70.090 | 16.080 | −5.010 | 1.00 | 27.91 | N |
| ATOM | 1068 | CZ | ARG | A | 166 | 71.277 | 15.551 | −5.274 | 1.00 | 28.25 | C |
| ATOM | 1069 | NH1 | ARG | A | 166 | 72.327 | 16.299 | −5.636 | 1.00 | 26.61 | N |
| ATOM | 1070 | NH2 | ARG | A | 166 | 71.404 | 14.246 | −5.142 | 1.00 | 25.73 | N |
| ATOM | 1071 | C | ARG | A | 166 | 68.284 | 20.261 | −1.348 | 1.00 | 17.04 | C |
| ATOM | 1072 | O | ARG | A | 166 | 68.778 | 19.491 | −0.517 | 1.00 | 17.97 | O |
| ATOM | 1073 | N | ASP | A | 167 | 68.165 | 21.571 | −1.127 | 1.00 | 16.44 | N |
| ATOM | 1074 | CA | ASP | A | 167 | 68.537 | 22.157 | 0.172 | 1.00 | 17.08 | C |
| ATOM | 1075 | CB | ASP | A | 167 | 70.018 | 22.615 | 0.164 | 1.00 | 16.76 | C |
| ATOM | 1076 | CG | ASP | A | 167 | 70.639 | 22.733 | 1.576 | 1.00 | 19.52 | C |
| ATOM | 1077 | OD1 | ASP | A | 167 | 70.136 | 22.109 | 2.552 | 1.00 | 19.71 | O |
| ATOM | 1078 | OD2 | ASP | A | 167 | 71.660 | 23.441 | 1.792 | 1.00 | 20.05 | O |
| ATOM | 1079 | C | ASP | A | 167 | 67.593 | 23.303 | 0.559 | 1.00 | 16.55 | C |
| ATOM | 1080 | O | ASP | A | 167 | 68.028 | 24.327 | 1.065 | 1.00 | 17.85 | O |
| ATOM | 1081 | N | ILE | A | 168 | 66.289 | 23.130 | 0.321 | 1.00 | 16.37 | N |
| ATOM | 1082 | CA | ILE | A | 168 | 65.304 | 24.165 | 0.643 | 1.00 | 15.43 | C |
| ATOM | 1083 | CB | ILE | A | 168 | 63.919 | 23.803 | 0.061 | 1.00 | 15.49 | C |
| ATOM | 1084 | CG1 | ILE | A | 168 | 63.990 | 23.685 | −1.467 | 1.00 | 15.11 | C |
| ATOM | 1085 | CD1 | ILE | A | 168 | 62.816 | 22.891 | −2.049 | 1.00 | 15.98 | C |
| ATOM | 1086 | CG2 | ILE | A | 168 | 62.841 | 24.821 | 0.481 | 1.00 | 14.46 | C |
| ATOM | 1087 | C | ILE | A | 168 | 65.226 | 24.257 | 2.159 | 1.00 | 15.89 | C |
| ATOM | 1088 | O | ILE | A | 168 | 64.988 | 23.247 | 2.828 | 1.00 | 15.71 | O |
| ATOM | 1089 | N | LYS | A | 169 | 65.445 | 25.459 | 2.682 | 1.00 | 15.34 | N |
| ATOM | 1090 | CA | LYS | A | 169 | 65.458 | 25.724 | 4.116 | 1.00 | 15.97 | C |
| ATOM | 1091 | CB | LYS | A | 169 | 66.666 | 25.055 | 4.793 | 1.00 | 16.17 | C |
| ATOM | 1092 | CG | LYS | A | 169 | 68.018 | 25.601 | 4.321 | 1.00 | 18.35 | C |
| ATOM | 1093 | CD | LYS | A | 169 | 69.165 | 24.636 | 4.595 | 1.00 | 21.69 | C |
| ATOM | 1094 | CE | LYS | A | 169 | 69.449 | 24.497 | 6.073 | 1.00 | 23.35 | C |
| ATOM | 1095 | NZ | LYS | A | 169 | 70.883 | 24.061 | 6.239 | 1.00 | 24.28 | N |
| ATOM | 1096 | C | LYS | A | 169 | 65.542 | 27.234 | 4.314 | 1.00 | 15.57 | C |
| ATOM | 1097 | O | LYS | A | 169 | 65.954 | 27.971 | 3.392 | 1.00 | 15.50 | O |
| ATOM | 1098 | N | ASP | A | 170 | 65.213 | 27.676 | 5.526 | 1.00 | 15.04 | N |
| ATOM | 1099 | CA | ASP | A | 170 | 65.179 | 29.090 | 5.868 | 1.00 | 15.81 | C |
| ATOM | 1100 | CB | ASP | A | 170 | 64.849 | 29.284 | 7.358 | 1.00 | 15.73 | C |
| ATOM | 1101 | CG | ASP | A | 170 | 65.734 | 28.457 | 8.295 | 1.00 | 18.50 | C |
| ATOM | 1102 | OD1 | ASP | A | 170 | 66.780 | 27.869 | 7.880 | 1.00 | 19.79 | O |
| ATOM | 1103 | OD2 | ASP | A | 170 | 65.450 | 28.361 | 9.509 | 1.00 | 21.07 | O |
| ATOM | 1104 | C | ASP | A | 170 | 66.433 | 29.880 | 5.468 | 1.00 | 16.21 | C |
| ATOM | 1105 | O | ASP | A | 170 | 66.321 | 30.954 | 4.874 | 1.00 | 16.22 | O |
| ATOM | 1106 | N | GLU | A | 171 | 67.607 | 29.341 | 5.792 | 1.00 | 16.57 | N |
| ATOM | 1107 | CA | GLU | A | 171 | 68.918 | 29.951 | 5.480 | 1.00 | 17.89 | C |
| ATOM | 1108 | CB | GLU | A | 171 | 70.040 | 28.959 | 5.796 | 1.00 | 18.41 | C |
| ATOM | 1109 | CG | GLU | A | 171 | 70.770 | 29.167 | 7.082 | 1.00 | 24.87 | C |
| ATOM | 1110 | CD | GLU | A | 171 | 71.735 | 28.024 | 7.352 | 1.00 | 29.19 | C |
| ATOM | 1111 | OE1 | GLU | A | 171 | 72.124 | 27.876 | 8.521 | 1.00 | 34.95 | O |
| ATOM | 1112 | OE2 | GLU | A | 171 | 72.072 | 27.259 | 6.407 | 1.00 | 30.15 | O |
| ATOM | 1113 | C | GLU | A | 171 | 69.096 | 30.224 | 3.998 | 1.00 | 16.68 | C |
| ATOM | 1114 | O | GLU | A | 171 | 69.853 | 31.125 | 3.626 | 1.00 | 15.52 | O |
| ATOM | 1115 | N | ASN | A | 172 | 68.468 | 29.391 | 3.171 | 1.00 | 15.92 | N |
| ATOM | 1116 | CA | ASN | A | 172 | 68.601 | 29.487 | 1.707 | 1.00 | 15.37 | C |
| ATOM | 1117 | CB | ASN | A | 172 | 68.806 | 28.111 | 1.093 | 1.00 | 14.94 | C |
| ATOM | 1118 | CG | ASN | A | 172 | 70.122 | 27.517 | 1.493 | 1.00 | 15.14 | C |
| ATOM | 1119 | OD1 | ASN | A | 172 | 71.047 | 28.264 | 1.760 | 1.00 | 15.76 | O |
| ATOM | 1120 | ND2 | ASN | A | 172 | 70.218 | 26.188 | 1.567 | 1.00 | 13.68 | N |
| ATOM | 1121 | C | ASN | A | 172 | 67.454 | 30.228 | 1.026 | 1.00 | 15.23 | C |
| ATOM | 1122 | O | ASN | A | 172 | 67.198 | 30.038 | −0.154 | 1.00 | 15.24 | O |
| ATOM | 1123 | N | ILE | A | 173 | 66.799 | 31.102 | 1.778 | 1.00 | 15.43 | N |
| ATOM | 1124 | CA | ILE | A | 173 | 65.714 | 31.920 | 1.252 | 1.00 | 15.78 | C |
| ATOM | 1125 | CB | ILE | A | 173 | 64.350 | 31.416 | 1.775 | 1.00 | 15.76 | C |
| ATOM | 1126 | CG1 | ILE | A | 173 | 64.066 | 29.987 | 1.287 | 1.00 | 16.39 | C |
| ATOM | 1127 | CD1 | ILE | A | 173 | 62.948 | 29.264 | 2.080 | 1.00 | 14.60 | C |
| ATOM | 1128 | CG2 | ILE | A | 173 | 63.211 | 32.396 | 1.379 | 1.00 | 15.41 | C |
| ATOM | 1129 | C | ILE | A | 173 | 65.947 | 33.363 | 1.701 | 1.00 | 15.85 | C |
| ATOM | 1130 | O | ILE | A | 173 | 66.149 | 33.622 | 2.884 | 1.00 | 15.12 | O |
| ATOM | 1131 | N | LEU | A | 174 | 65.914 | 34.285 | 0.741 | 1.00 | 16.29 | N |
| ATOM | 1132 | CA | LEU | A | 174 | 66.139 | 35.707 | 0.992 | 1.00 | 16.81 | C |
| ATOM | 1133 | CB | LEU | A | 174 | 67.035 | 36.307 | −0.105 | 1.00 | 16.84 | C |
| ATOM | 1134 | CG | LEU | A | 174 | 68.479 | 35.805 | −0.189 | 1.00 | 17.08 | C |
| ATOM | 1135 | CD1 | LEU | A | 174 | 69.217 | 36.628 | −1.214 | 1.00 | 16.20 | C |
| ATOM | 1136 | CD2 | LEU | A | 174 | 69.187 | 35.865 | 1.153 | 1.00 | 16.61 | C |
| ATOM | 1137 | C | LEU | A | 174 | 64.816 | 36.419 | 0.956 | 1.00 | 17.35 | C |
| ATOM | 1138 | O | LEU | A | 174 | 63.963 | 36.085 | 0.127 | 1.00 | 17.94 | O |
| ATOM | 1139 | N | ILE | A | 175 | 64.641 | 37.387 | 1.850 | 1.00 | 17.74 | N |
| ATOM | 1140 | CA | ILE | A | 175 | 63.470 | 38.255 | 1.833 | 1.00 | 18.68 | C |
| ATOM | 1141 | CB | ILE | A | 175 | 62.818 | 38.360 | 3.226 | 1.00 | 18.50 | C |
| ATOM | 1142 | CG1 | ILE | A | 175 | 62.456 | 36.976 | 3.794 | 1.00 | 18.87 | C |
| ATOM | 1143 | CD1 | ILE | A | 175 | 62.302 | 36.995 | 5.327 | 1.00 | 18.86 | C |
| ATOM | 1144 | CG2 | ILE | A | 175 | 61.576 | 39.278 | 3.167 | 1.00 | 18.77 | C |
| ATOM | 1145 | C | ILE | A | 175 | 63.902 | 39.649 | 1.389 | 1.00 | 19.58 | C |
| ATOM | 1146 | O | ILE | A | 175 | 64.664 | 40.322 | 2.095 | 1.00 | 19.31 | O |
| ATOM | 1147 | N | ASP | A | 176 | 63.412 | 40.074 | 0.228 | 1.00 | 20.23 | N |
| ATOM | 1148 | CA | ASP | A | 176 | 63.581 | 41.449 | −0.230 | 1.00 | 21.84 | C |
| ATOM | 1149 | CB | ASP | A | 176 | 63.315 | 41.541 | −1.739 | 1.00 | 22.01 | C |
| ATOM | 1150 | CG | ASP | A | 176 | 63.414 | 42.967 | −2.280 | 1.00 | 23.60 | C |
| ATOM | 1151 | OD1 | ASP | A | 176 | 63.243 | 43.920 | −1.502 | 1.00 | 23.50 | O |
| ATOM | 1152 | OD2 | ASP | A | 176 | 63.625 | 43.217 | −3.482 | 1.00 | 24.73 | O |
| ATOM | 1153 | C | ASP | A | 176 | 62.588 | 42.286 | 0.587 | 1.00 | 22.79 | C |
| ATOM | 1154 | O | ASP | A | 176 | 61.387 | 42.316 | 0.297 | 1.00 | 22.81 | O |
| ATOM | 1155 | N | LEU | A | 177 | 63.102 | 42.924 | 1.634 | 1.00 | 23.58 | N |
| ATOM | 1156 | CA | LEU | A | 177 | 62.271 | 43.537 | 2.660 | 1.00 | 24.93 | C |
| ATOM | 1157 | CB | LEU | A | 177 | 63.132 | 44.012 | 3.835 | 1.00 | 25.33 | C |
| ATOM | 1158 | CG | LEU | A | 177 | 63.764 | 42.908 | 4.700 | 1.00 | 25.91 | C |
| ATOM | 1159 | CD1 | LEU | A | 177 | 64.830 | 43.473 | 5.621 | 1.00 | 26.41 | C |
| ATOM | 1160 | CD2 | LEU | A | 177 | 62.715 | 42.118 | 5.504 | 1.00 | 27.00 | C |
| ATOM | 1161 | C | LEU | A | 177 | 61.345 | 44.658 | 2.164 | 1.00 | 25.53 | C |
| ATOM | 1162 | O | LEU | A | 177 | 60.231 | 44.789 | 2.661 | 1.00 | 26.29 | O |
| ATOM | 1163 | N | ASN | A | 178 | 61.789 | 45.433 | 1.177 | 1.00 | 25.87 | N |
| ATOM | 1164 | CA | ASN | A | 178 | 60.985 | 46.525 | 0.607 | 1.00 | 26.30 | C |
| ATOM | 1165 | CB | ASN | A | 178 | 61.875 | 47.492 | −0.187 | 1.00 | 26.74 | C |
| ATOM | 1166 | CG | ASN | A | 178 | 62.544 | 48.534 | 0.690 | 1.00 | 28.73 | C |
| ATOM | 1167 | OD1 | ASN | A | 178 | 62.308 | 48.600 | 1.904 | 1.00 | 31.92 | O |
| ATOM | 1168 | ND2 | ASN | A | 178 | 63.382 | 49.361 | 0.078 | 1.00 | 30.86 | N |
| ATOM | 1169 | C | ASN | A | 178 | 59.857 | 46.042 | −0.307 | 1.00 | 26.01 | C |
| ATOM | 1170 | O | ASN | A | 178 | 58.771 | 46.630 | −0.338 | 1.00 | 25.84 | O |
| ATOM | 1171 | N | ARG | A | 179 | 60.134 | 44.986 | −1.066 | 1.00 | 25.22 | N |
| ATOM | 1172 | CA | ARG | A | 179 | 59.202 | 44.497 | −2.073 | 1.00 | 25.06 | C |
| ATOM | 1173 | CB | ARG | A | 179 | 59.951 | 44.089 | −3.340 | 1.00 | 25.43 | C |
| ATOM | 1174 | CG | ARG | A | 179 | 60.482 | 45.270 | −4.157 | 1.00 | 26.18 | C |
| ATOM | 1175 | CD | ARG | A | 179 | 61.170 | 44.845 | −5.426 | 1.00 | 29.81 | C |
| ATOM | 1176 | NE | ARG | A | 179 | 61.712 | 45.951 | −6.219 | 1.00 | 33.67 | N |
| ATOM | 1177 | CZ | ARG | A | 179 | 60.987 | 46.869 | −6.859 | 1.00 | 35.31 | C |
| ATOM | 1178 | NH1 | ARG | A | 179 | 59.658 | 46.854 | −6.805 | 1.00 | 36.83 | N |
| ATOM | 1179 | NH2 | ARG | A | 179 | 61.598 | 47.816 | −7.559 | 1.00 | 36.82 | N |
| ATOM | 1180 | C | ARG | A | 179 | 58.330 | 43.355 | −1.574 | 1.00 | 24.41 | C |
| ATOM | 1181 | O | ARG | A | 179 | 57.345 | 43.004 | −2.221 | 1.00 | 24.91 | O |
| ATOM | 1182 | N | GLY | A | 180 | 58.675 | 42.786 | −0.421 | 1.00 | 23.59 | N |
| ATOM | 1183 | CA | GLY | A | 180 | 57.977 | 41.611 | 0.083 | 1.00 | 22.56 | C |
| ATOM | 1184 | C | GLY | A | 180 | 58.160 | 40.359 | −0.779 | 1.00 | 21.90 | C |
| ATOM | 1185 | O | GLY | A | 180 | 57.320 | 39.456 | −0.754 | 1.00 | 21.19 | O |
| ATOM | 1186 | N | GLU | A | 181 | 59.263 | 40.310 | −1.524 | 1.00 | 21.18 | N |
| ATOM | 1187 | CA | GLU | A | 181 | 59.542 | 39.216 | −2.462 | 1.00 | 21.26 | C |
| ATOM | 1188 | CB | GLU | A | 181 | 60.001 | 39.767 | −3.815 | 1.00 | 20.99 | C |
| ATOM | 1189 | CG | GLU | A | 181 | 58.883 | 40.426 | −4.598 | 1.00 | 22.33 | C |
| ATOM | 1190 | CD | GLU | A | 181 | 59.360 | 41.165 | −5.827 | 1.00 | 24.38 | C |
| ATOM | 1191 | OE1 | GLU | A | 181 | 60.493 | 40.911 | −6.310 | 1.00 | 25.63 | O |
| ATOM | 1192 | OE2 | GLU | A | 181 | 58.578 | 42.011 | −6.317 | 1.00 | 26.99 | O |
| ATOM | 1193 | C | GLU | A | 181 | 60.613 | 38.281 | −1.928 | 1.00 | 20.80 | C |
| ATOM | 1194 | O | GLU | A | 181 | 61.659 | 38.735 | −1.467 | 1.00 | 20.59 | O |
| ATOM | 1195 | N | LEU | A | 182 | 60.348 | 36.979 | −2.002 | 1.00 | 20.60 | N |
| ATOM | 1196 | CA | LEU | A | 182 | 61.297 | 35.963 | −1.555 | 1.00 | 20.56 | C |
| ATOM | 1197 | CB | LEU | A | 182 | 60.570 | 34.791 | −0.891 | 1.00 | 20.37 | C |
| ATOM | 1198 | CG | LEU | A | 182 | 60.517 | 34.821 | 0.631 | 1.00 | 21.15 | C |
| ATOM | 1199 | CD1 | LEU | A | 182 | 59.818 | 36.090 | 1.096 | 1.00 | 22.41 | C |
| ATOM | 1200 | CD2 | LEU | A | 182 | 59.801 | 33.559 | 1.145 | 1.00 | 19.08 | C |
| ATOM | 1201 | C | LEU | A | 182 | 62.118 | 35.439 | −2.725 | 1.00 | 20.73 | C |
| ATOM | 1202 | O | LEU | A | 182 | 61.612 | 35.341 | −3.849 | 1.00 | 20.32 | O |
| ATOM | 1203 | N | LYS | A | 183 | 63.372 | 35.096 | −2.442 | 1.00 | 20.55 | N |
| ATOM | 1204 | CA | LYS | A | 183 | 64.313 | 34.617 | −3.448 | 1.00 | 20.89 | C |
| ATOM | 1205 | CB | LYS | A | 183 | 65.374 | 35.692 | −3.759 | 1.00 | 21.35 | C |
| ATOM | 1206 | CG | LYS | A | 183 | 64.883 | 36.741 | −4.750 | 1.00 | 24.94 | C |
| ATOM | 1207 | CD | LYS | A | 183 | 65.757 | 37.973 | −4.773 | 1.00 | 26.84 | C |
| ATOM | 1208 | CE | LYS | A | 183 | 65.777 | 38.639 | −6.149 | 1.00 | 30.89 | C |
| ATOM | 1209 | NZ | LYS | A | 183 | 64.436 | 38.886 | −6.765 | 1.00 | 30.16 | N |
| ATOM | 1210 | C | LYS | A | 183 | 65.005 | 33.373 | −2.927 | 1.00 | 19.72 | C |
| ATOM | 1211 | O | LYS | A | 183 | 65.572 | 33.384 | −1.844 | 1.00 | 19.28 | O |
| ATOM | 1212 | N | LEU | A | 184 | 64.960 | 32.317 | −3.725 | 1.00 | 18.78 | N |
| ATOM | 1213 | CA | LEU | A | 184 | 65.689 | 31.088 | −3.462 | 1.00 | 18.80 | C |
| ATOM | 1214 | CB | LEU | A | 184 | 65.057 | 29.957 | −4.278 | 1.00 | 18.65 | C |
| ATOM | 1215 | CG | LEU | A | 184 | 65.182 | 28.479 | −3.925 | 1.00 | 22.15 | C |
| ATOM | 1216 | CD1 | LEU | A | 184 | 64.840 | 28.132 | −2.445 | 1.00 | 22.78 | C |
| ATOM | 1217 | CD2 | LEU | A | 184 | 64.302 | 27.643 | −4.894 | 1.00 | 19.46 | C |
| ATOM | 1218 | C | LEU | A | 184 | 67.166 | 31.270 | −3.827 | 1.00 | 17.97 | C |
| ATOM | 1219 | O | LEU | A | 184 | 67.500 | 31.813 | −4.895 | 1.00 | 17.51 | O |
| ATOM | 1220 | N | ILE | A | 185 | 68.048 | 30.840 | −2.932 | 1.00 | 16.83 | N |
| ATOM | 1221 | CA | ILE | A | 185 | 69.482 | 30.849 | −3.204 | 1.00 | 16.25 | C |
| ATOM | 1222 | CB | ILE | A | 185 | 70.215 | 31.922 | −2.348 | 1.00 | 16.56 | C |
| ATOM | 1223 | CG1 | ILE | A | 185 | 69.892 | 31.720 | −0.859 | 1.00 | 15.91 | C |
| ATOM | 1224 | CD1 | ILE | A | 185 | 70.811 | 32.451 | 0.107 | 1.00 | 16.54 | C |
| ATOM | 1225 | CG2 | ILE | A | 185 | 69.924 | 33.333 | −2.865 | 1.00 | 15.65 | C |
| ATOM | 1226 | C | ILE | A | 185 | 70.098 | 29.504 | −2.880 | 1.00 | 16.22 | C |
| ATOM | 1227 | O | ILE | A | 185 | 69.411 | 28.607 | −2.380 | 1.00 | 16.13 | O |
| ATOM | 1228 | N | ASP | A | 186 | 71.409 | 29.408 | −3.127 | 1.00 | 15.92 | N |
| ATOM | 1229 | CA | ASP | A | 186 | 72.254 | 28.263 | −2.788 | 1.00 | 15.84 | C |
| ATOM | 1230 | CB | ASP | A | 186 | 72.344 | 28.039 | −1.269 | 1.00 | 16.13 | C |
| ATOM | 1231 | CG | ASP | A | 186 | 73.351 | 26.972 | −0.898 | 1.00 | 15.73 | C |
| ATOM | 1232 | OD1 | ASP | A | 186 | 73.977 | 26.372 | −1.791 | 1.00 | 17.28 | O |
| ATOM | 1233 | OD2 | ASP | A | 186 | 73.571 | 26.621 | 0.268 | 1.00 | 16.60 | O |
| ATOM | 1234 | C | ASP | A | 186 | 71.875 | 26.980 | −3.505 | 1.00 | 17.14 | C |
| ATOM | 1235 | O | ASP | A | 186 | 71.185 | 26.128 | −2.968 | 1.00 | 17.68 | O |
| ATOM | 1236 | N | PHE | A | 187 | 72.372 | 26.828 | −4.721 | 1.00 | 17.98 | N |
| ATOM | 1237 | CA | PHE | A | 187 | 72.163 | 25.603 | −5.459 | 1.00 | 19.11 | C |
| ATOM | 1238 | CB | PHE | A | 187 | 71.813 | 25.935 | −6.905 | 1.00 | 19.00 | C |
| ATOM | 1239 | CG | PHE | A | 187 | 70.462 | 26.572 | −7.042 | 1.00 | 18.98 | C |
| ATOM | 1240 | CD1 | PHE | A | 187 | 70.277 | 27.914 | −6.731 | 1.00 | 17.58 | C |
| ATOM | 1241 | CE1 | PHE | A | 187 | 69.010 | 28.508 | −6.832 | 1.00 | 19.96 | C |
| ATOM | 1242 | CZ | PHE | A | 187 | 67.917 | 27.743 | −7.260 | 1.00 | 19.45 | C |
| ATOM | 1243 | CE2 | PHE | A | 187 | 68.094 | 26.402 | −7.575 | 1.00 | 18.98 | C |
| ATOM | 1244 | CD2 | PHE | A | 187 | 69.366 | 25.817 | −7.452 | 1.00 | 19.42 | C |
| ATOM | 1245 | C | PHE | A | 187 | 73.367 | 24.669 | −5.342 | 1.00 | 19.81 | C |
| ATOM | 1246 | O | PHE | A | 187 | 73.540 | 23.776 | −6.162 | 1.00 | 20.32 | O |
| ATOM | 1247 | N | GLY | A | 188 | 74.157 | 24.864 | −4.285 | 1.00 | 20.15 | N |
| ATOM | 1248 | CA | GLY | A | 188 | 75.355 | 24.074 | −4.027 | 1.00 | 20.64 | C |
| ATOM | 1249 | C | GLY | A | 188 | 75.130 | 22.581 | −3.824 | 1.00 | 20.89 | C |
| ATOM | 1250 | O | GLY | A | 188 | 76.061 | 21.795 | −4.008 | 1.00 | 20.70 | O |
| ATOM | 1251 | N | SER | A | 189 | 73.904 | 22.186 | −3.460 | 1.00 | 20.61 | N |
| ATOM | 1252 | CA | SER | A | 189 | 73.585 | 20.774 | −3.205 | 1.00 | 20.36 | C |
| ATOM | 1253 | CB | SER | A | 189 | 72.891 | 20.598 | −1.843 | 1.00 | 20.68 | C |
| ATOM | 1254 | OG | SER | A | 189 | 73.701 | 21.035 | −0.766 | 1.00 | 20.77 | O |
| ATOM | 1255 | C | SER | A | 189 | 72.668 | 20.218 | −4.276 | 1.00 | 20.23 | C |
| ATOM | 1256 | O | SER | A | 189 | 72.229 | 19.079 | −4.181 | 1.00 | 19.67 | O |
| ATOM | 1257 | N | GLY | A | 190 | 72.359 | 21.040 | −5.273 | 1.00 | 19.66 | N |
| ATOM | 1258 | CA | GLY | A | 190 | 71.362 | 20.701 | −6.256 | 1.00 | 20.11 | C |
| ATOM | 1259 | C | GLY | A | 190 | 71.779 | 19.655 | −7.282 | 1.00 | 20.35 | C |
| ATOM | 1260 | O | GLY | A | 190 | 72.924 | 19.203 | −7.309 | 1.00 | 20.22 | O |
| ATOM | 1261 | N | ALA | A | 191 | 70.830 | 19.271 | −8.122 | 1.00 | 19.81 | N |
| ATOM | 1262 | CA | ALA | A | 191 | 71.085 | 18.339 | −9.201 | 1.00 | 20.13 | C |
| ATOM | 1263 | CB | ALA | A | 191 | 70.902 | 16.875 | −8.722 | 1.00 | 20.04 | C |
| ATOM | 1264 | C | ALA | A | 191 | 70.130 | 18.652 | −10.323 | 1.00 | 20.23 | C |
| ATOM | 1265 | O | ALA | A | 191 | 69.126 | 19.344 | −10.122 | 1.00 | 20.11 | O |
| ATOM | 1266 | N | LEU | A | 192 | 70.446 | 18.144 | −11.512 | 1.00 | 20.33 | N |
| ATOM | 1267 | CA | LEU | A | 192 | 69.504 | 18.154 | −12.617 | 1.00 | 20.48 | C |
| ATOM | 1268 | CB | LEU | A | 192 | 70.160 | 17.538 | −13.870 | 1.00 | 20.65 | C |
| ATOM | 1269 | CG | LEU | A | 192 | 71.394 | 18.241 | −14.460 | 1.00 | 21.30 | C |
| ATOM | 1270 | CD1 | LEU | A | 192 | 72.025 | 17.441 | −15.650 | 1.00 | 24.01 | C |
| ATOM | 1271 | CD2 | LEU | A | 192 | 71.028 | 19.649 | −14.922 | 1.00 | 20.92 | C |
| ATOM | 1272 | C | LEU | A | 192 | 68.301 | 17.329 | −12.171 | 1.00 | 20.62 | C |
| ATOM | 1273 | O | LEU | A | 192 | 68.472 | 16.302 | −11.520 | 1.00 | 20.87 | O |
| ATOM | 1274 | N | LEU | A | 193 | 67.092 | 17.787 | −12.488 | 1.00 | 20.82 | N |
| ATOM | 1275 | CA | LEU | A | 193 | 65.883 | 17.033 | −12.163 | 1.00 | 20.67 | C |
| ATOM | 1276 | CB | LEU | A | 193 | 64.626 | 17.901 | −12.333 | 1.00 | 20.90 | C |
| ATOM | 1277 | CG | LEU | A | 193 | 63.271 | 17.308 | −11.915 | 1.00 | 21.37 | C |
| ATOM | 1278 | CD1 | LEU | A | 193 | 63.216 | 16.979 | −10.428 | 1.00 | 19.30 | C |
| ATOM | 1279 | CD2 | LEU | A | 193 | 62.103 | 18.226 | −12.303 | 1.00 | 23.02 | C |
| ATOM | 1280 | C | LEU | A | 193 | 65.770 | 15.784 | −13.042 | 1.00 | 20.94 | C |
| ATOM | 1281 | O | LEU | A | 193 | 66.005 | 15.837 | −14.250 | 1.00 | 20.46 | O |
| ATOM | 1282 | N | LYS | A | 194 | 65.403 | 14.666 | −12.423 | 1.00 | 20.70 | N |
| ATOM | 1283 | CA | LYS | A | 194 | 65.191 | 13.411 | −13.140 | 1.00 | 20.09 | C |
| ATOM | 1284 | CB | LYS | A | 194 | 66.464 | 12.559 | −13.094 | 1.00 | 20.03 | C |
| ATOM | 1285 | CG | LYS | A | 194 | 66.780 | 11.998 | −11.717 | 1.00 | 18.07 | C |
| ATOM | 1286 | CD | LYS | A | 194 | 68.169 | 11.391 | −11.691 | 1.00 | 19.71 | C |
| ATOM | 1287 | CE | LYS | A | 194 | 68.381 | 10.652 | −10.385 | 1.00 | 20.42 | C |
| ATOM | 1288 | NZ | LYS | A | 194 | 69.586 | 9.789 | −10.403 | 1.00 | 19.70 | N |
| ATOM | 1289 | C | LYS | A | 194 | 64.025 | 12.658 | −12.505 | 1.00 | 19.94 | C |
| ATOM | 1290 | O | LYS | A | 194 | 63.669 | 12.913 | −11.349 | 1.00 | 19.51 | O |
| ATOM | 1291 | N | ASP | A | 195 | 63.456 | 11.722 | −13.260 | 1.00 | 19.71 | N |
| ATOM | 1292 | CA | ASP | A | 195 | 62.308 | 10.943 | −12.808 | 1.00 | 19.96 | C |
| ATOM | 1293 | CB | ASP | A | 195 | 61.352 | 10.706 | −13.972 | 1.00 | 20.12 | C |
| ATOM | 1294 | CG | ASP | A | 195 | 60.843 | 12.005 | −14.573 | 1.00 | 21.01 | C |
| ATOM | 1295 | OD1 | ASP | A | 195 | 60.213 | 12.792 | −13.832 | 1.00 | 21.96 | O |
| ATOM | 1296 | OD2 | ASP | A | 195 | 61.028 | 12.311 | −15.770 | 1.00 | 22.33 | O |
| ATOM | 1297 | C | ASP | A | 195 | 62.684 | 9.613 | −12.166 | 1.00 | 20.21 | C |
| ATOM | 1298 | O | ASP | A | 195 | 61.811 | 8.866 | −11.740 | 1.00 | 20.16 | O |
| ATOM | 1299 | N | THR | A | 196 | 63.979 | 9.324 | −12.111 | 1.00 | 20.34 | N |
| ATOM | 1300 | CA | THR | A | 196 | 64.459 | 8.086 | −11.519 | 1.00 | 20.80 | C |
| ATOM | 1301 | CB | THR | A | 196 | 65.550 | 7.433 | −12.402 | 1.00 | 20.79 | C |
| ATOM | 1302 | OG1 | THR | A | 196 | 66.489 | 8.431 | −12.832 | 1.00 | 19.71 | O |
| ATOM | 1303 | CG2 | THR | A | 196 | 64.942 | 6.891 | −13.701 | 1.00 | 20.94 | C |
| ATOM | 1304 | C | THR | A | 196 | 64.997 | 8.357 | −10.132 | 1.00 | 21.32 | C |
| ATOM | 1305 | O | THR | A | 196 | 65.059 | 9.515 | −9.686 | 1.00 | 21.17 | O |
| ATOM | 1306 | N | VAL | A | 197 | 65.387 | 7.290 | −9.447 | 1.00 | 21.71 | N |
| ATOM | 1307 | CA | VAL | A | 197 | 65.741 | 7.385 | −8.039 | 1.00 | 22.75 | C |
| ATOM | 1308 | CB | VAL | A | 197 | 65.661 | 5.983 | −7.364 | 1.00 | 22.95 | C |
| ATOM | 1309 | CG1 | VAL | A | 197 | 66.823 | 5.098 | −7.798 | 1.00 | 24.30 | C |
| ATOM | 1310 | CG2 | VAL | A | 197 | 65.592 | 6.094 | −5.849 | 1.00 | 23.66 | C |
| ATOM | 1311 | C | VAL | A | 197 | 67.102 | 8.074 | −7.834 | 1.00 | 22.86 | C |
| ATOM | 1312 | O | VAL | A | 197 | 68.044 | 7.862 | −8.611 | 1.00 | 23.08 | O |
| ATOM | 1313 | N | TYR | A | 198 | 67.176 | 8.939 | −6.823 | 1.00 | 22.60 | N |
| ATOM | 1314 | CA | TYR | A | 198 | 68.441 | 9.506 | −6.375 | 1.00 | 22.42 | C |
| ATOM | 1315 | CB | TYR | A | 198 | 68.242 | 10.922 | −5.829 | 1.00 | 21.98 | C |
| ATOM | 1316 | CG | TYR | A | 198 | 67.927 | 11.966 | −6.869 | 1.00 | 19.83 | C |
| ATOM | 1317 | CD1 | TYR | A | 198 | 66.610 | 12.197 | −7.262 | 1.00 | 18.45 | C |
| ATOM | 1318 | CE1 | TYR | A | 198 | 66.301 | 13.147 | −8.205 | 1.00 | 16.56 | C |
| ATOM | 1319 | CZ | TYR | A | 198 | 67.307 | 13.909 | −8.772 | 1.00 | 17.06 | C |
| ATOM | 1320 | OH | TYR | A | 198 | 66.949 | 14.848 | −9.713 | 1.00 | 15.18 | O |
| ATOM | 1321 | CE2 | TYR | A | 198 | 68.641 | 13.708 | −8.410 | 1.00 | 16.87 | C |
| ATOM | 1322 | CD2 | TYR | A | 198 | 68.942 | 12.732 | −7.455 | 1.00 | 18.51 | C |
| ATOM | 1323 | C | TYR | A | 198 | 69.010 | 8.631 | −5.266 | 1.00 | 22.99 | C |
| ATOM | 1324 | O | TYR | A | 198 | 68.273 | 8.209 | −4.363 | 1.00 | 22.53 | O |
| ATOM | 1325 | N | THR | A | 199 | 70.314 | 8.370 | −5.337 | 1.00 | 23.94 | N |
| ATOM | 1326 | CA | THR | A | 199 | 71.034 | 7.617 | −4.302 | 1.00 | 25.22 | C |
| ATOM | 1327 | CB | THR | A | 199 | 71.694 | 6.331 | −4.880 | 1.00 | 25.19 | C |
| ATOM | 1328 | OG1 | THR | A | 199 | 72.604 | 6.688 | −5.923 | 1.00 | 25.62 | O |
| ATOM | 1329 | CG2 | THR | A | 199 | 70.681 | 5.427 | −5.571 | 1.00 | 25.21 | C |
| ATOM | 1330 | C | THR | A | 199 | 72.111 | 8.475 | −3.635 | 1.00 | 26.00 | C |
| ATOM | 1331 | O | THR | A | 199 | 72.881 | 7.982 | −2.818 | 1.00 | 25.92 | O |
| ATOM | 1332 | N | ASP | A | 200 | 72.170 | 9.752 | −4.007 | 1.00 | 27.25 | N |
| ATOM | 1333 | CA | ASP | A | 200 | 73.121 | 10.694 | −3.424 | 1.00 | 28.69 | C |
| ATOM | 1334 | CB | ASP | A | 200 | 74.132 | 11.197 | −4.477 | 1.00 | 29.29 | C |
| ATOM | 1335 | CG | ASP | A | 200 | 73.490 | 12.085 | −5.559 | 1.00 | 32.11 | C |
| ATOM | 1336 | OD1 | ASP | A | 200 | 73.879 | 13.277 | −5.649 | 1.00 | 34.36 | O |
| ATOM | 1337 | OD2 | ASP | A | 200 | 72.609 | 11.686 | −6.370 | 1.00 | 34.31 | O |
| ATOM | 1338 | C | ASP | A | 200 | 72.374 | 11.855 | −2.788 | 1.00 | 28.79 | C |
| ATOM | 1339 | O | ASP | A | 200 | 71.327 | 12.278 | −3.283 | 1.00 | 28.34 | O |
| ATOM | 1340 | N | PHE | A | 201 | 72.904 | 12.350 | −1.677 | 1.00 | 29.39 | N |
| ATOM | 1341 | CA | PHE | A | 201 | 72.328 | 13.501 | −1.000 | 1.00 | 30.23 | C |
| ATOM | 1342 | CB | PHE | A | 201 | 71.140 | 13.077 | −0.140 | 1.00 | 29.94 | C |
| ATOM | 1343 | CG | PHE | A | 201 | 70.534 | 14.196 | 0.660 | 1.00 | 28.00 | C |
| ATOM | 1344 | CD1 | PHE | A | 201 | 69.676 | 15.109 | 0.062 | 1.00 | 27.14 | C |
| ATOM | 1345 | CE1 | PHE | A | 201 | 69.104 | 16.143 | 0.791 | 1.00 | 27.27 | C |
| ATOM | 1346 | CZ | PHE | A | 201 | 69.381 | 16.266 | 2.148 | 1.00 | 27.00 | C |
| ATOM | 1347 | CE2 | PHE | A | 201 | 70.244 | 15.357 | 2.763 | 1.00 | 28.69 | C |
| ATOM | 1348 | CD2 | PHE | A | 201 | 70.811 | 14.322 | 2.012 | 1.00 | 28.58 | C |
| ATOM | 1349 | C | PHE | A | 201 | 73.381 | 14.192 | −0.146 | 1.00 | 31.40 | C |
| ATOM | 1350 | O | PHE | A | 201 | 74.097 | 13.542 | 0.614 | 1.00 | 31.87 | O |
| ATOM | 1351 | N | ASP | A | 202 | 73.449 | 15.512 | −0.274 | 1.00 | 32.09 | N |
| ATOM | 1352 | CA | ASP | A | 202 | 74.428 | 16.314 | 0.432 | 1.00 | 33.18 | C |
| ATOM | 1353 | CB | ASP | A | 202 | 75.581 | 16.677 | −0.510 | 1.00 | 34.28 | C |
| ATOM | 1354 | CG | ASP | A | 202 | 76.918 | 16.282 | 0.054 | 1.00 | 37.84 | C |
| ATOM | 1355 | OD1 | ASP | A | 202 | 77.292 | 15.090 | −0.089 | 1.00 | 42.28 | O |
| ATOM | 1356 | OD2 | ASP | A | 202 | 77.655 | 17.087 | 0.671 | 1.00 | 41.24 | O |
| ATOM | 1357 | C | ASP | A | 202 | 73.823 | 17.576 | 1.024 | 1.00 | 32.23 | C |
| ATOM | 1358 | O | ASP | A | 202 | 74.550 | 18.471 | 1.451 | 1.00 | 32.66 | O |
| ATOM | 1359 | N | GLY | A | 203 | 72.494 | 17.648 | 1.049 | 1.00 | 31.25 | N |
| ATOM | 1360 | CA | GLY | A | 203 | 71.801 | 18.764 | 1.677 | 1.00 | 29.41 | C |
| ATOM | 1361 | C | GLY | A | 203 | 71.721 | 18.616 | 3.189 | 1.00 | 28.69 | C |
| ATOM | 1362 | O | GLY | A | 203 | 72.489 | 17.863 | 3.792 | 1.00 | 28.32 | O |
| ATOM | 1363 | N | THR | A | 204 | 70.770 | 19.324 | 3.800 | 1.00 | 28.21 | N |
| ATOM | 1364 | CA | THR | A | 204 | 70.628 | 19.353 | 5.257 | 1.00 | 27.26 | C |
| ATOM | 1365 | CB | THR | A | 204 | 69.950 | 20.656 | 5.702 | 1.00 | 26.96 | C |
| ATOM | 1366 | OG1 | THR | A | 204 | 70.654 | 21.769 | 5.142 | 1.00 | 26.09 | O |
| ATOM | 1367 | CG2 | THR | A | 204 | 70.103 | 20.855 | 7.222 | 1.00 | 25.58 | C |
| ATOM | 1368 | C | THR | A | 204 | 69.847 | 18.152 | 5.776 | 1.00 | 27.62 | C |
| ATOM | 1369 | O | THR | A | 204 | 68.680 | 17.948 | 5.397 | 1.00 | 27.26 | O |
| ATOM | 1370 | N | ARG | A | 205 | 70.483 | 17.391 | 6.670 | 1.00 | 27.57 | N |
| ATOM | 1371 | CA | ARG | A | 205 | 69.928 | 16.139 | 7.173 | 1.00 | 27.70 | C |
| ATOM | 1372 | CB | ARG | A | 205 | 70.881 | 15.491 | 8.193 | 1.00 | 28.59 | C |
| ATOM | 1373 | CG | ARG | A | 205 | 70.306 | 14.238 | 8.883 | 1.00 | 31.06 | C |
| ATOM | 1374 | CD | ARG | A | 205 | 71.326 | 13.172 | 9.299 | 1.00 | 34.02 | C |
| ATOM | 1375 | NE | ARG | A | 205 | 71.717 | 12.397 | 8.132 | 1.00 | 37.36 | N |
| ATOM | 1376 | CZ | ARG | A | 205 | 71.619 | 11.073 | 7.997 | 1.00 | 37.19 | C |
| ATOM | 1377 | NH1 | ARG | A | 205 | 71.156 | 10.302 | 8.970 | 1.00 | 36.97 | N |
| ATOM | 1378 | NH2 | ARG | A | 205 | 71.995 | 10.523 | 6.856 | 1.00 | 36.80 | N |
| ATOM | 1379 | C | ARG | A | 205 | 68.508 | 16.270 | 7.748 | 1.00 | 27.42 | C |
| ATOM | 1380 | O | ARG | A | 205 | 67.600 | 15.482 | 7.393 | 1.00 | 27.39 | O |
| ATOM | 1381 | N | VAL | A | 206 | 68.323 | 17.271 | 8.611 | 1.00 | 26.15 | N |
| ATOM | 1382 | CA | VAL | A | 206 | 67.088 | 17.452 | 9.368 | 1.00 | 24.94 | C |
| ATOM | 1383 | CB | VAL | A | 206 | 67.268 | 18.408 | 10.593 | 1.00 | 25.07 | C |
| ATOM | 1384 | CG1 | VAL | A | 206 | 68.149 | 17.763 | 11.651 | 1.00 | 24.65 | C |
| ATOM | 1385 | CG2 | VAL | A | 206 | 67.842 | 19.792 | 10.167 | 1.00 | 23.92 | C |
| ATOM | 1386 | C | VAL | A | 206 | 65.986 | 17.956 | 8.455 | 1.00 | 25.16 | C |
| ATOM | 1387 | O | VAL | A | 206 | 64.835 | 18.077 | 8.883 | 1.00 | 25.56 | O |
| ATOM | 1388 | N | TYR | A | 207 | 66.343 | 18.226 | 7.195 | 1.00 | 24.00 | N |
| ATOM | 1389 | CA | TYR | A | 207 | 65.363 | 18.533 | 6.169 | 1.00 | 23.56 | C |
| ATOM | 1390 | CB | TYR | A | 207 | 65.792 | 19.777 | 5.388 | 1.00 | 23.98 | C |
| ATOM | 1391 | CG | TYR | A | 207 | 65.472 | 21.091 | 6.067 | 1.00 | 23.10 | C |
| ATOM | 1392 | CD1 | TYR | A | 207 | 66.274 | 21.587 | 7.101 | 1.00 | 22.69 | C |
| ATOM | 1393 | CE1 | TYR | A | 207 | 65.980 | 22.819 | 7.722 | 1.00 | 24.39 | C |
| ATOM | 1394 | CZ | TYR | A | 207 | 64.884 | 23.551 | 7.277 | 1.00 | 27.17 | C |
| ATOM | 1395 | OH | TYR | A | 207 | 64.556 | 24.776 | 7.844 | 1.00 | 30.13 | O |
| ATOM | 1396 | CE2 | TYR | A | 207 | 64.080 | 23.064 | 6.243 | 1.00 | 25.83 | C |
| ATOM | 1397 | CD2 | TYR | A | 207 | 64.382 | 21.851 | 5.647 | 1.00 | 24.73 | C |
| ATOM | 1398 | C | TYR | A | 207 | 65.141 | 17.371 | 5.198 | 1.00 | 22.93 | C |
| ATOM | 1399 | O | TYR | A | 207 | 64.299 | 17.469 | 4.285 | 1.00 | 22.62 | O |
| ATOM | 1400 | N | SER | A | 208 | 65.906 | 16.292 | 5.382 | 1.00 | 21.96 | N |
| ATOM | 1401 | CA | SER | A | 208 | 65.849 | 15.131 | 4.487 | 1.00 | 21.81 | C |
| ATOM | 1402 | CB | SER | A | 208 | 67.203 | 14.408 | 4.432 | 1.00 | 22.13 | C |
| ATOM | 1403 | OG | SER | A | 208 | 67.426 | 13.650 | 5.611 | 1.00 | 23.96 | O |
| ATOM | 1404 | C | SER | A | 208 | 64.738 | 14.139 | 4.876 | 1.00 | 20.92 | C |
| ATOM | 1405 | O | SER | A | 208 | 64.427 | 13.970 | 6.062 | 1.00 | 20.62 | O |
| ATOM | 1406 | N | PRO | A | 209 | 64.177 | 13.466 | 3.873 | 1.00 | 19.97 | N |
| ATOM | 1407 | CA | PRO | A | 209 | 62.999 | 12.620 | 4.067 | 1.00 | 19.59 | C |
| ATOM | 1408 | CB | PRO | A | 209 | 62.467 | 12.452 | 2.639 | 1.00 | 19.55 | C |
| ATOM | 1409 | CG | PRO | A | 209 | 63.689 | 12.524 | 1.774 | 1.00 | 19.80 | C |
| ATOM | 1410 | CD | PRO | A | 209 | 64.644 | 13.447 | 2.470 | 1.00 | 19.85 | C |
| ATOM | 1411 | C | PRO | A | 209 | 63.380 | 11.269 | 4.690 | 1.00 | 19.33 | C |
| ATOM | 1412 | O | PRO | A | 209 | 64.554 | 10.867 | 4.623 | 1.00 | 19.13 | O |
| ATOM | 1413 | N | PRO | A | 210 | 62.415 | 10.592 | 5.304 | 1.00 | 18.92 | N |
| ATOM | 1414 | CA | PRO | A | 210 | 62.668 | 9.300 | 5.961 | 1.00 | 19.08 | C |
| ATOM | 1415 | CB | PRO | A | 210 | 61.301 | 8.921 | 6.557 | 1.00 | 18.83 | C |
| ATOM | 1416 | CG | PRO | A | 210 | 60.302 | 9.737 | 5.821 | 1.00 | 19.11 | C |
| ATOM | 1417 | CD | PRO | A | 210 | 61.006 | 11.018 | 5.435 | 1.00 | 18.89 | C |
| ATOM | 1418 | C | PRO | A | 210 | 63.164 | 8.203 | 5.012 | 1.00 | 19.74 | C |
| ATOM | 1419 | O | PRO | A | 210 | 63.892 | 7.324 | 5.476 | 1.00 | 19.91 | O |
| ATOM | 1420 | N | GLU | A | 211 | 62.796 | 8.256 | 3.732 | 1.00 | 19.73 | N |
| ATOM | 1421 | CA | GLU | A | 211 | 63.273 | 7.278 | 2.766 | 1.00 | 20.85 | C |
| ATOM | 1422 | CB | GLU | A | 211 | 62.461 | 7.323 | 1.451 | 1.00 | 20.53 | C |
| ATOM | 1423 | CG | GLU | A | 211 | 62.554 | 8.649 | 0.684 | 1.00 | 20.57 | C |
| ATOM | 1424 | CD | GLU | A | 211 | 61.446 | 9.651 | 1.014 | 1.00 | 20.44 | C |
| ATOM | 1425 | OE1 | GLU | A | 211 | 60.905 | 9.640 | 2.143 | 1.00 | 21.24 | O |
| ATOM | 1426 | OE2 | GLU | A | 211 | 61.122 | 10.474 | 0.132 | 1.00 | 19.92 | O |
| ATOM | 1427 | C | GLU | A | 211 | 64.782 | 7.446 | 2.532 | 1.00 | 21.48 | C |
| ATOM | 1428 | O | GLU | A | 211 | 65.491 | 6.465 | 2.284 | 1.00 | 21.77 | O |
| ATOM | 1429 | N | TRP | A | 212 | 65.269 | 8.683 | 2.624 | 1.00 | 21.85 | N |
| ATOM | 1430 | CA | TRP | A | 212 | 66.702 | 8.917 | 2.576 | 1.00 | 22.59 | C |
| ATOM | 1431 | CB | TRP | A | 212 | 67.059 | 10.402 | 2.439 | 1.00 | 21.76 | C |
| ATOM | 1432 | CG | TRP | A | 212 | 68.537 | 10.610 | 2.665 | 1.00 | 23.30 | C |
| ATOM | 1433 | CD1 | TRP | A | 212 | 69.128 | 11.209 | 3.745 | 1.00 | 23.54 | C |
| ATOM | 1434 | NE1 | TRP | A | 212 | 70.497 | 11.187 | 3.610 | 1.00 | 24.29 | N |
| ATOM | 1435 | CE2 | TRP | A | 212 | 70.828 | 10.556 | 2.441 | 1.00 | 23.65 | C |
| ATOM | 1436 | CD2 | TRP | A | 212 | 69.618 | 10.169 | 1.817 | 1.00 | 22.66 | C |
| ATOM | 1437 | CE3 | TRP | A | 212 | 69.684 | 9.497 | 0.589 | 1.00 | 22.34 | C |
| ATOM | 1438 | CZ3 | TRP | A | 212 | 70.944 | 9.227 | 0.028 | 1.00 | 23.22 | C |
| ATOM | 1439 | CH2 | TRP | A | 212 | 72.129 | 9.621 | 0.680 | 1.00 | 23.33 | C |
| ATOM | 1440 | CZ2 | TRP | A | 212 | 72.093 | 10.288 | 1.882 | 1.00 | 24.31 | C |
| ATOM | 1441 | C | TRP | A | 212 | 67.375 | 8.324 | 3.814 | 1.00 | 22.99 | C |
| ATOM | 1442 | O | TRP | A | 212 | 68.368 | 7.609 | 3.695 | 1.00 | 23.46 | O |
| ATOM | 1443 | N | ILE | A | 213 | 66.812 | 8.611 | 4.986 | 1.00 | 23.54 | N |
| ATOM | 1444 | CA | ILE | A | 213 | 67.375 | 8.166 | 6.265 | 1.00 | 24.57 | C |
| ATOM | 1445 | CB | ILE | A | 213 | 66.566 | 8.729 | 7.468 | 1.00 | 24.25 | C |
| ATOM | 1446 | CG1 | ILE | A | 213 | 66.550 | 10.265 | 7.469 | 1.00 | 24.58 | C |
| ATOM | 1447 | CD1 | ILE | A | 213 | 67.945 | 10.927 | 7.479 | 1.00 | 24.84 | C |
| ATOM | 1448 | CG2 | ILE | A | 213 | 67.143 | 8.217 | 8.788 | 1.00 | 24.24 | C |
| ATOM | 1449 | C | ILE | A | 213 | 67.480 | 6.646 | 6.350 | 1.00 | 25.27 | C |
| ATOM | 1450 | O | ILE | A | 213 | 68.523 | 6.113 | 6.741 | 1.00 | 25.42 | O |
| ATOM | 1451 | N | ARG | A | 214 | 66.409 | 5.963 | 5.955 | 1.00 | 26.19 | N |
| ATOM | 1452 | CA | ARG | A | 214 | 66.330 | 4.508 | 6.035 | 1.00 | 27.54 | C |
| ATOM | 1453 | CB | ARG | A | 214 | 64.873 | 4.052 | 6.126 | 1.00 | 27.97 | C |
| ATOM | 1454 | CG | ARG | A | 214 | 64.138 | 4.599 | 7.344 | 1.00 | 31.59 | C |
| ATOM | 1455 | CD | ARG | A | 214 | 62.621 | 4.436 | 7.296 | 1.00 | 37.14 | C |
| ATOM | 1456 | NE | ARG | A | 214 | 62.201 | 3.037 | 7.213 | 1.00 | 40.55 | N |
| ATOM | 1457 | CZ | ARG | A | 214 | 62.233 | 2.167 | 8.219 | 1.00 | 43.40 | C |
| ATOM | 1458 | NH1 | ARG | A | 214 | 62.672 | 2.525 | 9.423 | 1.00 | 44.08 | N |
| ATOM | 1459 | NH2 | ARG | A | 214 | 61.823 | 0.919 | 8.018 | 1.00 | 44.82 | N |
| ATOM | 1460 | C | ARG | A | 214 | 67.018 | 3.787 | 4.877 | 1.00 | 27.77 | C |
| ATOM | 1461 | O | ARG | A | 214 | 67.799 | 2.869 | 5.113 | 1.00 | 27.88 | O |
| ATOM | 1462 | N | TYR | A | 215 | 66.731 | 4.195 | 3.641 | 1.00 | 27.94 | N |
| ATOM | 1463 | CA | TYR | A | 215 | 67.151 | 3.428 | 2.460 | 1.00 | 28.52 | C |
| ATOM | 1464 | CB | TYR | A | 215 | 65.931 | 2.983 | 1.642 | 1.00 | 28.63 | C |
| ATOM | 1465 | CG | TYR | A | 215 | 64.788 | 2.478 | 2.486 | 1.00 | 30.63 | C |
| ATOM | 1466 | CD1 | TYR | A | 215 | 64.884 | 1.262 | 3.177 | 1.00 | 32.35 | C |
| ATOM | 1467 | CE1 | TYR | A | 215 | 63.832 | 0.800 | 3.965 | 1.00 | 33.17 | C |
| ATOM | 1468 | CZ | TYR | A | 215 | 62.674 | 1.560 | 4.063 | 1.00 | 34.20 | C |
| ATOM | 1469 | OH | TYR | A | 215 | 61.625 | 1.121 | 4.834 | 1.00 | 36.84 | O |
| ATOM | 1470 | CE2 | TYR | A | 215 | 62.556 | 2.764 | 3.390 | 1.00 | 33.32 | C |
| ATOM | 1471 | CD2 | TYR | A | 215 | 63.610 | 3.217 | 2.607 | 1.00 | 32.43 | C |
| ATOM | 1472 | C | TYR | A | 215 | 68.143 | 4.128 | 1.539 | 1.00 | 28.30 | C |
| ATOM | 1473 | O | TYR | A | 215 | 68.551 | 3.558 | 0.531 | 1.00 | 28.64 | O |
| ATOM | 1474 | N | HIS | A | 216 | 68.520 | 5.360 | 1.870 | 1.00 | 27.98 | N |
| ATOM | 1475 | CA | HIS | A | 216 | 69.431 | 6.133 | 1.027 | 1.00 | 27.92 | C |
| ATOM | 1476 | CB | HIS | A | 216 | 70.866 | 5.574 | 1.125 | 1.00 | 28.78 | C |
| ATOM | 1477 | CG | HIS | A | 216 | 71.628 | 6.080 | 2.315 | 1.00 | 32.47 | C |
| ATOM | 1478 | ND1 | HIS | A | 216 | 72.993 | 5.922 | 2.453 | 1.00 | 36.05 | N |
| ATOM | 1479 | CE1 | HIS | A | 216 | 73.386 | 6.479 | 3.587 | 1.00 | 37.27 | C |
| ATOM | 1480 | NE2 | HIS | A | 216 | 72.328 | 6.997 | 4.187 | 1.00 | 36.76 | N |
| ATOM | 1481 | CD2 | HIS | A | 216 | 71.217 | 6.761 | 3.413 | 1.00 | 35.02 | C |
| ATOM | 1482 | C | HIS | A | 216 | 68.936 | 6.268 | −0.435 | 1.00 | 26.74 | C |
| ATOM | 1483 | O | HIS | A | 216 | 69.728 | 6.295 | −1.383 | 1.00 | 26.90 | O |
| ATOM | 1484 | N | ARG | A | 217 | 67.616 | 6.360 | −0.592 | 1.00 | 25.13 | N |
| ATOM | 1485 | CA | ARG | A | 217 | 66.964 | 6.461 | −1.892 | 1.00 | 24.20 | C |
| ATOM | 1486 | CB | ARG | A | 217 | 66.380 | 5.106 | −2.341 | 1.00 | 24.35 | C |
| ATOM | 1487 | CG | ARG | A | 217 | 67.373 | 3.964 | −2.525 | 1.00 | 27.36 | C |
| ATOM | 1488 | CD | ARG | A | 217 | 66.718 | 2.584 | −2.668 | 1.00 | 31.36 | C |
| ATOM | 1489 | NE | ARG | A | 217 | 66.048 | 2.433 | −3.958 | 1.00 | 34.40 | N |
| ATOM | 1490 | CZ | ARG | A | 217 | 66.633 | 1.967 | −5.061 | 1.00 | 36.63 | C |
| ATOM | 1491 | NH1 | ARG | A | 217 | 67.909 | 1.595 | −5.043 | 1.00 | 36.66 | N |
| ATOM | 1492 | NH2 | ARG | A | 217 | 65.943 | 1.879 | −6.190 | 1.00 | 37.21 | N |
| ATOM | 1493 | C | ARG | A | 217 | 65.808 | 7.429 | −1.749 | 1.00 | 22.74 | C |
| ATOM | 1494 | O | ARG | A | 217 | 65.124 | 7.420 | −0.729 | 1.00 | 23.06 | O |
| ATOM | 1495 | N | TYR | A | 218 | 65.580 | 8.240 | −2.777 | 1.00 | 20.60 | N |
| ATOM | 1496 | CA | TYR | A | 218 | 64.445 | 9.141 | −2.824 | 1.00 | 18.74 | C |
| ATOM | 1497 | CB | TYR | A | 218 | 64.674 | 10.371 | −1.917 | 1.00 | 18.22 | C |
| ATOM | 1498 | CG | TYR | A | 218 | 65.867 | 11.216 | −2.299 | 1.00 | 16.94 | C |
| ATOM | 1499 | CD1 | TYR | A | 218 | 67.160 | 10.880 | −1.870 | 1.00 | 15.03 | C |
| ATOM | 1500 | CE1 | TYR | A | 218 | 68.265 | 11.679 | −2.220 | 1.00 | 14.80 | C |
| ATOM | 1501 | CZ | TYR | A | 218 | 68.064 | 12.802 | −3.020 | 1.00 | 15.81 | C |
| ATOM | 1502 | OH | TYR | A | 218 | 69.125 | 13.594 | −3.393 | 1.00 | 16.21 | O |
| ATOM | 1503 | CE2 | TYR | A | 218 | 66.790 | 13.145 | −3.453 | 1.00 | 15.34 | C |
| ATOM | 1504 | CD2 | TYR | A | 218 | 65.705 | 12.355 | −3.093 | 1.00 | 15.11 | C |
| ATOM | 1505 | C | TYR | A | 218 | 64.212 | 9.584 | −4.267 | 1.00 | 18.07 | C |
| ATOM | 1506 | O | TYR | A | 218 | 65.112 | 9.486 | −5.102 | 1.00 | 17.75 | O |
| ATOM | 1507 | N | HIS | A | 219 | 63.006 | 10.075 | −4.545 | 1.00 | 17.04 | N |
| ATOM | 1508 | CA | HIS | A | 219 | 62.721 | 10.757 | −5.811 | 1.00 | 16.74 | C |
| ATOM | 1509 | CB | HIS | A | 219 | 61.430 | 10.231 | −6.440 | 1.00 | 16.49 | C |
| ATOM | 1510 | CG | HIS | A | 219 | 61.547 | 8.819 | −6.917 | 1.00 | 18.35 | C |
| ATOM | 1511 | ND1 | HIS | A | 219 | 61.677 | 8.489 | −8.253 | 1.00 | 18.97 | N |
| ATOM | 1512 | CE1 | HIS | A | 219 | 61.791 | 7.179 | −8.368 | 1.00 | 17.84 | C |
| ATOM | 1513 | NE2 | HIS | A | 219 | 61.763 | 6.650 | −7.157 | 1.00 | 19.10 | N |
| ATOM | 1514 | CD2 | HIS | A | 219 | 61.621 | 7.653 | −6.230 | 1.00 | 16.82 | C |
| ATOM | 1515 | C | HIS | A | 219 | 62.651 | 12.255 | −5.551 | 1.00 | 16.15 | C |
| ATOM | 1516 | O | HIS | A | 219 | 62.346 | 12.681 | −4.438 | 1.00 | 15.76 | O |
| ATOM | 1517 | N | GLY | A | 220 | 62.938 | 13.048 | −6.576 | 1.00 | 16.23 | N |
| ATOM | 1518 | CA | GLY | A | 220 | 63.172 | 14.462 | −6.384 | 1.00 | 16.70 | C |
| ATOM | 1519 | C | GLY | A | 220 | 61.992 | 15.217 | −5.807 | 1.00 | 16.87 | C |
| ATOM | 1520 | O | GLY | A | 220 | 62.110 | 15.886 | −4.788 | 1.00 | 16.14 | O |
| ATOM | 1521 | N | ARG | A | 221 | 60.848 | 15.097 | −6.469 | 1.00 | 17.28 | N |
| ATOM | 1522 | CA | ARG | A | 221 | 59.691 | 15.923 | −6.150 | 1.00 | 17.64 | C |
| ATOM | 1523 | CB | ARG | A | 221 | 58.590 | 15.746 | −7.185 | 1.00 | 18.45 | C |
| ATOM | 1524 | CG | ARG | A | 221 | 59.002 | 16.190 | −8.578 | 1.00 | 23.24 | C |
| ATOM | 1525 | CD | ARG | A | 221 | 58.156 | 15.591 | −9.697 | 1.00 | 28.55 | C |
| ATOM | 1526 | NE | ARG | A | 221 | 58.705 | 15.933 | −11.013 | 1.00 | 32.56 | N |
| ATOM | 1527 | CZ | ARG | A | 221 | 59.512 | 15.147 | −11.720 | 1.00 | 35.42 | C |
| ATOM | 1528 | NH1 | ARG | A | 221 | 59.879 | 13.956 | −11.241 | 1.00 | 36.89 | N |
| ATOM | 1529 | NH2 | ARG | A | 221 | 59.943 | 15.539 | −12.920 | 1.00 | 35.53 | N |
| ATOM | 1530 | C | ARG | A | 221 | 59.155 | 15.652 | −4.764 | 1.00 | 16.82 | C |
| ATOM | 1531 | O | ARG | A | 221 | 58.922 | 16.596 | −4.015 | 1.00 | 16.95 | O |
| ATOM | 1532 | N | SER | A | 222 | 58.981 | 14.374 | −4.417 | 1.00 | 15.97 | N |
| ATOM | 1533 | CA | SER | A | 222 | 58.439 | 14.002 | −3.111 | 1.00 | 15.37 | C |
| ATOM | 1534 | CB | SER | A | 222 | 58.036 | 12.510 | −3.066 | 1.00 | 15.54 | C |
| ATOM | 1535 | OG | SER | A | 222 | 59.136 | 11.654 | −3.333 | 1.00 | 15.91 | O |
| ATOM | 1536 | C | SER | A | 222 | 59.396 | 14.347 | −1.971 | 1.00 | 14.74 | C |
| ATOM | 1537 | O | SER | A | 222 | 58.963 | 14.684 | −0.874 | 1.00 | 14.86 | O |
| ATOM | 1538 | N | ALA | A | 223 | 60.694 | 14.270 | −2.225 | 1.00 | 14.48 | N |
| ATOM | 1539 | CA | ALA | A | 223 | 61.686 | 14.738 | −1.253 | 1.00 | 14.36 | C |
| ATOM | 1540 | CB | ALA | A | 223 | 63.081 | 14.313 | −1.677 | 1.00 | 14.45 | C |
| ATOM | 1541 | C | ALA | A | 223 | 61.619 | 16.266 | −1.091 | 1.00 | 14.53 | C |
| ATOM | 1542 | O | ALA | A | 223 | 61.718 | 16.779 | 0.030 | 1.00 | 14.59 | O |
| ATOM | 1543 | N | ALA | A | 224 | 61.441 | 16.982 | −2.205 | 1.00 | 13.88 | N |
| ATOM | 1544 | CA | ALA | A | 224 | 61.310 | 18.444 | −2.169 | 1.00 | 13.96 | C |
| ATOM | 1545 | CB | ALA | A | 224 | 61.174 | 19.032 | −3.591 | 1.00 | 13.04 | C |
| ATOM | 1546 | C | ALA | A | 224 | 60.116 | 18.832 | −1.296 | 1.00 | 13.72 | C |
| ATOM | 1547 | O | ALA | A | 224 | 60.220 | 19.713 | −0.462 | 1.00 | 14.14 | O |
| ATOM | 1548 | N | VAL | A | 225 | 59.001 | 18.123 | −1.470 | 1.00 | 14.13 | N |
| ATOM | 1549 | CA | VAL | A | 225 | 57.776 | 18.363 | −0.704 | 1.00 | 13.34 | C |
| ATOM | 1550 | CB | VAL | A | 225 | 56.602 | 17.517 | −1.301 | 1.00 | 13.99 | C |
| ATOM | 1551 | CG1 | VAL | A | 225 | 55.370 | 17.457 | −0.352 | 1.00 | 11.55 | C |
| ATOM | 1552 | CG2 | VAL | A | 225 | 56.236 | 18.063 | −2.701 | 1.00 | 12.97 | C |
| ATOM | 1553 | C | VAL | A | 225 | 57.986 | 18.076 | 0.778 | 1.00 | 14.00 | C |
| ATOM | 1554 | O | VAL | A | 225 | 57.513 | 18.820 | 1.650 | 1.00 | 14.66 | O |
| ATOM | 1555 | N | TRP | A | 226 | 58.695 | 16.996 | 1.087 | 1.00 | 13.82 | N |
| ATOM | 1556 | CA | TRP | A | 226 | 59.037 | 16.751 | 2.481 | 1.00 | 14.22 | C |
| ATOM | 1557 | CB | TRP | A | 226 | 59.908 | 15.501 | 2.626 | 1.00 | 14.10 | C |
| ATOM | 1558 | CG | TRP | A | 226 | 60.362 | 15.305 | 4.045 | 1.00 | 14.21 | C |
| ATOM | 1559 | CD1 | TRP | A | 226 | 61.444 | 15.888 | 4.651 | 1.00 | 13.01 | C |
| ATOM | 1560 | NE1 | TRP | A | 226 | 61.516 | 15.488 | 5.961 | 1.00 | 13.97 | N |
| ATOM | 1561 | CE2 | TRP | A | 226 | 60.473 | 14.643 | 6.231 | 1.00 | 13.33 | C |
| ATOM | 1562 | CD2 | TRP | A | 226 | 59.723 | 14.510 | 5.046 | 1.00 | 14.66 | C |
| ATOM | 1563 | CE3 | TRP | A | 226 | 58.583 | 13.683 | 5.063 | 1.00 | 14.53 | C |
| ATOM | 1564 | CZ3 | TRP | A | 226 | 58.254 | 13.024 | 6.238 | 1.00 | 14.25 | C |
| ATOM | 1565 | CH2 | TRP | A | 226 | 59.020 | 13.173 | 7.395 | 1.00 | 14.22 | C |
| ATOM | 1566 | CZ2 | TRP | A | 226 | 60.132 | 13.981 | 7.415 | 1.00 | 15.55 | C |
| ATOM | 1567 | C | TRP | A | 226 | 59.763 | 17.976 | 3.062 | 1.00 | 13.86 | C |
| ATOM | 1568 | O | TRP | A | 226 | 59.406 | 18.468 | 4.136 | 1.00 | 14.16 | O |
| ATOM | 1569 | N | SER | A | 227 | 60.764 | 18.485 | 2.349 | 1.00 | 13.20 | N |
| ATOM | 1570 | CA | SER | A | 227 | 61.522 | 19.610 | 2.875 | 1.00 | 13.52 | C |
| ATOM | 1571 | CB | SER | A | 227 | 62.793 | 19.886 | 2.043 | 1.00 | 13.28 | C |
| ATOM | 1572 | OG | SER | A | 227 | 62.448 | 20.474 | 0.803 | 1.00 | 12.96 | O |
| ATOM | 1573 | C | SER | A | 227 | 60.634 | 20.845 | 2.967 | 1.00 | 13.72 | C |
| ATOM | 1574 | O | SER | A | 227 | 60.848 | 21.702 | 3.816 | 1.00 | 13.60 | O |
| ATOM | 1575 | N | LEU | A | 228 | 59.614 | 20.917 | 2.110 | 1.00 | 13.17 | N |
| ATOM | 1576 | CA | LEU | A | 228 | 58.673 | 22.028 | 2.171 | 1.00 | 13.14 | C |
| ATOM | 1577 | CB | LEU | A | 228 | 57.807 | 22.105 | 0.891 | 1.00 | 12.32 | C |
| ATOM | 1578 | CG | LEU | A | 228 | 58.606 | 22.646 | −0.297 | 1.00 | 11.69 | C |
| ATOM | 1579 | CD1 | LEU | A | 228 | 57.931 | 22.321 | −1.659 | 1.00 | 13.88 | C |
| ATOM | 1580 | CD2 | LEU | A | 228 | 58.893 | 24.135 | −0.171 | 1.00 | 10.58 | C |
| ATOM | 1581 | C | LEU | A | 228 | 57.801 | 21.968 | 3.427 | 1.00 | 12.15 | C |
| ATOM | 1582 | O | LEU | A | 228 | 57.489 | 22.990 | 4.020 | 1.00 | 12.32 | O |
| ATOM | 1583 | N | GLY | A | 229 | 57.405 | 20.766 | 3.811 | 1.00 | 12.02 | N |
| ATOM | 1584 | CA | GLY | A | 229 | 56.693 | 20.556 | 5.056 | 1.00 | 12.19 | C |
| ATOM | 1585 | C | GLY | A | 229 | 57.512 | 20.973 | 6.281 | 1.00 | 12.64 | C |
| ATOM | 1586 | O | GLY | A | 229 | 56.968 | 21.560 | 7.224 | 1.00 | 12.42 | O |
| ATOM | 1587 | N | ILE | A | 230 | 58.811 | 20.662 | 6.269 | 1.00 | 13.07 | N |
| ATOM | 1588 | CA | ILE | A | 230 | 59.718 | 21.050 | 7.357 | 1.00 | 13.99 | C |
| ATOM | 1589 | CB | ILE | A | 230 | 61.145 | 20.421 | 7.133 | 1.00 | 13.90 | C |
| ATOM | 1590 | CG1 | ILE | A | 230 | 61.067 | 18.894 | 7.053 | 1.00 | 13.25 | C |
| ATOM | 1591 | CD1 | ILE | A | 230 | 60.622 | 18.243 | 8.368 | 1.00 | 11.51 | C |
| ATOM | 1592 | CG2 | ILE | A | 230 | 62.118 | 20.815 | 8.272 | 1.00 | 14.90 | C |
| ATOM | 1593 | C | ILE | A | 230 | 59.785 | 22.587 | 7.409 | 1.00 | 14.39 | C |
| ATOM | 1594 | O | ILE | A | 230 | 59.686 | 23.211 | 8.488 | 1.00 | 14.11 | O |
| ATOM | 1595 | N | LEU | A | 231 | 59.915 | 23.182 | 6.222 | 1.00 | 14.04 | N |
| ATOM | 1596 | CA | LEU | A | 231 | 59.961 | 24.620 | 6.083 | 1.00 | 14.10 | C |
| ATOM | 1597 | CB | LEU | A | 231 | 60.197 | 24.995 | 4.609 | 1.00 | 14.16 | C |
| ATOM | 1598 | CG | LEU | A | 231 | 60.121 | 26.508 | 4.330 | 1.00 | 15.24 | C |
| ATOM | 1599 | CD1 | LEU | A | 231 | 61.292 | 27.224 | 4.967 | 1.00 | 15.13 | C |
| ATOM | 1600 | CD2 | LEU | A | 231 | 60.075 | 26.778 | 2.838 | 1.00 | 15.78 | C |
| ATOM | 1601 | C | LEU | A | 231 | 58.688 | 25.299 | 6.615 | 1.00 | 14.01 | C |
| ATOM | 1602 | O | LEU | A | 231 | 58.775 | 26.270 | 7.382 | 1.00 | 13.50 | O |
| ATOM | 1603 | N | LEU | A | 232 | 57.515 | 24.796 | 6.217 | 1.00 | 13.22 | N |
| ATOM | 1604 | CA | LEU | A | 232 | 56.256 | 25.394 | 6.644 | 1.00 | 13.16 | C |
| ATOM | 1605 | CB | LEU | A | 232 | 55.031 | 24.750 | 5.949 | 1.00 | 13.56 | C |
| ATOM | 1606 | CG | LEU | A | 232 | 53.653 | 25.362 | 6.282 | 1.00 | 12.22 | C |
| ATOM | 1607 | CD1 | LEU | A | 232 | 53.627 | 26.902 | 6.159 | 1.00 | 13.83 | C |
| ATOM | 1608 | CD2 | LEU | A | 232 | 52.521 | 24.721 | 5.419 | 1.00 | 11.85 | C |
| ATOM | 1609 | C | LEU | A | 232 | 56.112 | 25.294 | 8.164 | 1.00 | 13.79 | C |
| ATOM | 1610 | O | LEU | A | 232 | 55.723 | 26.269 | 8.817 | 1.00 | 14.65 | O |
| ATOM | 1611 | N | TYR | A | 233 | 56.445 | 24.133 | 8.723 | 1.00 | 13.34 | N |
| ATOM | 1612 | CA | TYR | A | 233 | 56.407 | 23.940 | 10.175 | 1.00 | 13.68 | C |
| ATOM | 1613 | CB | TYR | A | 233 | 56.870 | 22.524 | 10.551 | 1.00 | 12.73 | C |
| ATOM | 1614 | CG | TYR | A | 233 | 56.786 | 22.263 | 12.044 | 1.00 | 14.51 | C |
| ATOM | 1615 | CD1 | TYR | A | 233 | 57.791 | 22.713 | 12.908 | 1.00 | 14.02 | C |
| ATOM | 1616 | CE1 | TYR | A | 233 | 57.728 | 22.487 | 14.266 | 1.00 | 13.74 | C |
| ATOM | 1617 | CZ | TYR | A | 233 | 56.647 | 21.796 | 14.798 | 1.00 | 15.95 | C |
| ATOM | 1618 | OH | TYR | A | 233 | 56.590 | 21.586 | 16.169 | 1.00 | 17.38 | O |
| ATOM | 1619 | CE2 | TYR | A | 233 | 55.630 | 21.352 | 13.978 | 1.00 | 15.69 | C |
| ATOM | 1620 | CD2 | TYR | A | 233 | 55.705 | 21.588 | 12.594 | 1.00 | 14.19 | C |
| ATOM | 1621 | C | TYR | A | 233 | 57.296 | 24.989 | 10.855 | 1.00 | 14.14 | C |
| ATOM | 1622 | O | TYR | A | 233 | 56.893 | 25.639 | 11.837 | 1.00 | 14.22 | O |
| ATOM | 1623 | N | ASP | A | 234 | 58.497 | 25.162 | 10.304 | 1.00 | 14.64 | N |
| ATOM | 1624 | CA | ASP | A | 234 | 59.462 | 26.128 | 10.820 | 1.00 | 14.70 | C |
| ATOM | 1625 | CB | ASP | A | 234 | 60.741 | 26.074 | 9.986 | 1.00 | 15.23 | C |
| ATOM | 1626 | CG | ASP | A | 234 | 61.806 | 27.031 | 10.482 | 1.00 | 17.95 | C |
| ATOM | 1627 | OD1 | ASP | A | 234 | 62.134 | 27.038 | 11.693 | 1.00 | 17.66 | O |
| ATOM | 1628 | OD2 | ASP | A | 234 | 62.372 | 27.815 | 9.707 | 1.00 | 23.64 | O |
| ATOM | 1629 | C | ASP | A | 234 | 58.902 | 27.550 | 10.842 | 1.00 | 15.14 | C |
| ATOM | 1630 | O | ASP | A | 234 | 59.130 | 28.304 | 11.808 | 1.00 | 14.44 | O |
| ATOM | 1631 | N | MET | A | 235 | 58.177 | 27.921 | 9.782 | 1.00 | 14.52 | N |
| ATOM | 1632 | CA | MET | A | 235 | 57.585 | 29.248 | 9.679 | 1.00 | 15.77 | C |
| ATOM | 1633 | CB | MET | A | 235 | 56.946 | 29.472 | 8.300 | 1.00 | 15.95 | C |
| ATOM | 1634 | CG | MET | A | 235 | 57.955 | 29.567 | 7.157 | 1.00 | 19.29 | C |
| ATOM | 1635 | SD | MET | A | 235 | 57.147 | 30.105 | 5.616 | 1.00 | 23.96 | S |
| ATOM | 1636 | CE | MET | A | 235 | 56.577 | 28.752 | 5.093 | 1.00 | 24.70 | C |
| ATOM | 1637 | C | MET | A | 235 | 56.535 | 29.503 | 10.756 | 1.00 | 15.44 | C |
| ATOM | 1638 | O | MET | A | 235 | 56.551 | 30.545 | 11.395 | 1.00 | 15.28 | O |
| ATOM | 1639 | N | VAL | A | 236 | 55.622 | 28.557 | 10.944 | 1.00 | 15.79 | N |
| ATOM | 1640 | CA | VAL | A | 236 | 54.480 | 28.780 | 11.845 | 1.00 | 16.11 | C |
| ATOM | 1641 | CB | VAL | A | 236 | 53.169 | 28.062 | 11.349 | 1.00 | 16.48 | C |
| ATOM | 1642 | CG1 | VAL | A | 236 | 52.709 | 28.622 | 9.995 | 1.00 | 15.52 | C |
| ATOM | 1643 | CG2 | VAL | A | 236 | 53.327 | 26.521 | 11.277 | 1.00 | 14.68 | C |
| ATOM | 1644 | C | VAL | A | 236 | 54.808 | 28.422 | 13.295 | 1.00 | 17.29 | C |
| ATOM | 1645 | O | VAL | A | 236 | 54.084 | 28.833 | 14.220 | 1.00 | 17.67 | O |
| ATOM | 1646 | N | CYS | A | 237 | 55.901 | 27.673 | 13.503 | 1.00 | 17.50 | N |
| ATOM | 1647 | CA | CYS | A | 237 | 56.276 | 27.261 | 14.863 | 1.00 | 18.98 | C |
| ATOM | 1648 | CB | CYS | A | 237 | 56.400 | 25.735 | 14.986 | 1.00 | 18.51 | C |
| ATOM | 1649 | SG | CYS | A | 237 | 54.825 | 24.891 | 14.842 | 1.00 | 22.03 | S |
| ATOM | 1650 | C | CYS | A | 237 | 57.548 | 27.914 | 15.366 | 1.00 | 18.98 | C |
| ATOM | 1651 | O | CYS | A | 237 | 57.788 | 27.936 | 16.562 | 1.00 | 18.75 | O |
| ATOM | 1652 | N | GLY | A | 238 | 58.359 | 28.443 | 14.452 | 1.00 | 19.38 | N |
| ATOM | 1653 | CA | GLY | A | 238 | 59.584 | 29.125 | 14.835 | 1.00 | 20.34 | C |
| ATOM | 1654 | C | GLY | A | 238 | 60.776 | 28.206 | 14.966 | 1.00 | 21.28 | C |
| ATOM | 1655 | O | GLY | A | 238 | 61.871 | 28.677 | 15.269 | 1.00 | 21.63 | O |
| ATOM | 1656 | N | ASP | A | 239 | 60.572 | 26.906 | 14.743 | 1.00 | 21.89 | N |
| ATOM | 1657 | CA | ASP | A | 239 | 61.662 | 25.904 | 14.741 | 1.00 | 23.14 | C |
| ATOM | 1658 | CB | ASP | A | 239 | 62.038 | 25.466 | 16.161 | 1.00 | 24.31 | C |
| ATOM | 1659 | CG | ASP | A | 239 | 63.557 | 25.367 | 16.363 | 1.00 | 28.93 | C |
| ATOM | 1660 | OD1 | ASP | A | 239 | 64.268 | 24.729 | 15.530 | 1.00 | 31.83 | O |
| ATOM | 1661 | OD2 | ASP | A | 239 | 64.126 | 25.919 | 17.333 | 1.00 | 34.08 | O |
| ATOM | 1662 | C | ASP | A | 239 | 61.271 | 24.671 | 13.918 | 1.00 | 22.10 | C |
| ATOM | 1663 | O | ASP | A | 239 | 60.110 | 24.508 | 13.582 | 1.00 | 22.05 | O |
| ATOM | 1664 | N | ILE | A | 240 | 62.241 | 23.823 | 13.590 | 1.00 | 21.41 | N |
| ATOM | 1665 | CA | ILE | A | 240 | 61.995 | 22.616 | 12.803 | 1.00 | 21.20 | C |
| ATOM | 1666 | CB | ILE | A | 240 | 63.299 | 22.130 | 12.119 | 1.00 | 21.27 | C |
| ATOM | 1667 | CG1 | ILE | A | 240 | 64.418 | 21.934 | 13.162 | 1.00 | 22.95 | C |
| ATOM | 1668 | CD1 | ILE | A | 240 | 65.727 | 21.359 | 12.604 | 1.00 | 24.55 | C |
| ATOM | 1669 | CG2 | ILE | A | 240 | 63.711 | 23.113 | 11.020 | 1.00 | 22.14 | C |
| ATOM | 1670 | C | ILE | A | 240 | 61.390 | 21.516 | 13.687 | 1.00 | 21.05 | C |
| ATOM | 1671 | O | ILE | A | 240 | 61.628 | 21.507 | 14.896 | 1.00 | 20.43 | O |
| ATOM | 1672 | N | PRO | A | 241 | 60.596 | 20.610 | 13.112 | 1.00 | 21.20 | N |
| ATOM | 1673 | CA | PRO | A | 241 | 59.885 | 19.609 | 13.924 | 1.00 | 22.14 | C |
| ATOM | 1674 | CB | PRO | A | 241 | 58.818 | 19.070 | 12.967 | 1.00 | 22.34 | C |
| ATOM | 1675 | CG | PRO | A | 241 | 59.418 | 19.243 | 11.581 | 1.00 | 20.54 | C |
| ATOM | 1676 | CD | PRO | A | 241 | 60.303 | 20.461 | 11.670 | 1.00 | 21.11 | C |
| ATOM | 1677 | C | PRO | A | 241 | 60.762 | 18.466 | 14.413 | 1.00 | 23.21 | C |
| ATOM | 1678 | O | PRO | A | 241 | 60.432 | 17.885 | 15.443 | 1.00 | 23.48 | O |
| ATOM | 1679 | N | PHE | A | 242 | 61.843 | 18.143 | 13.699 | 1.00 | 24.34 | N |
| ATOM | 1680 | CA | PHE | A | 242 | 62.625 | 16.949 | 13.999 | 1.00 | 25.31 | C |
| ATOM | 1681 | CB | PHE | A | 242 | 62.503 | 15.894 | 12.881 | 1.00 | 24.74 | C |
| ATOM | 1682 | CG | PHE | A | 242 | 61.097 | 15.596 | 12.440 | 1.00 | 22.55 | C |
| ATOM | 1683 | CD1 | PHE | A | 242 | 60.115 | 15.212 | 13.354 | 1.00 | 21.74 | C |
| ATOM | 1684 | CE1 | PHE | A | 242 | 58.812 | 14.916 | 12.923 | 1.00 | 21.18 | C |
| ATOM | 1685 | CZ | PHE | A | 242 | 58.489 | 15.011 | 11.556 | 1.00 | 21.06 | C |
| ATOM | 1686 | CE2 | PHE | A | 242 | 59.467 | 15.392 | 10.642 | 1.00 | 20.29 | C |
| ATOM | 1687 | CD2 | PHE | A | 242 | 60.763 | 15.672 | 11.088 | 1.00 | 21.16 | C |
| ATOM | 1688 | C | PHE | A | 242 | 64.099 | 17.286 | 14.169 | 1.00 | 27.27 | C |
| ATOM | 1689 | O | PHE | A | 242 | 64.671 | 18.038 | 13.370 | 1.00 | 27.35 | O |
| ATOM | 1690 | N | GLU | A | 243 | 64.716 | 16.692 | 15.186 | 1.00 | 29.13 | N |
| ATOM | 1691 | CA | GLU | A | 243 | 66.149 | 16.849 | 15.428 | 1.00 | 31.65 | C |
| ATOM | 1692 | CB | GLU | A | 243 | 66.404 | 17.361 | 16.849 | 1.00 | 32.51 | C |
| ATOM | 1693 | CG | GLU | A | 243 | 65.779 | 18.723 | 17.153 | 1.00 | 37.83 | C |
| ATOM | 1694 | CD | GLU | A | 243 | 66.521 | 19.895 | 16.505 | 1.00 | 43.98 | C |
| ATOM | 1695 | OE1 | GLU | A | 243 | 66.675 | 19.903 | 15.260 | 1.00 | 46.61 | O |
| ATOM | 1696 | OE2 | GLU | A | 243 | 66.941 | 20.824 | 17.241 | 1.00 | 46.61 | O |
| ATOM | 1697 | C | GLU | A | 243 | 66.951 | 15.568 | 15.187 | 1.00 | 31.62 | C |
| ATOM | 1698 | O | GLU | A | 243 | 68.096 | 15.630 | 14.759 | 1.00 | 32.81 | O |
| ATOM | 1699 | N | HIS | A | 244 | 66.361 | 14.409 | 15.454 | 1.00 | 31.55 | N |
| ATOM | 1700 | CA | HIS | A | 244 | 67.089 | 13.146 | 15.307 | 1.00 | 31.42 | C |
| ATOM | 1701 | CB | HIS | A | 244 | 67.187 | 12.423 | 16.650 | 1.00 | 32.01 | C |
| ATOM | 1702 | CG | HIS | A | 244 | 67.774 | 13.265 | 17.738 | 1.00 | 34.56 | C |
| ATOM | 1703 | ND1 | HIS | A | 244 | 67.014 | 13.790 | 18.763 | 1.00 | 36.67 | N |
| ATOM | 1704 | CE1 | HIS | A | 244 | 67.791 | 14.502 | 19.561 | 1.00 | 37.86 | C |
| ATOM | 1705 | NE2 | HIS | A | 244 | 69.026 | 14.462 | 19.087 | 1.00 | 37.93 | N |
| ATOM | 1706 | CD2 | HIS | A | 244 | 69.041 | 13.697 | 17.945 | 1.00 | 36.34 | C |
| ATOM | 1707 | C | HIS | A | 244 | 66.482 | 12.235 | 14.243 | 1.00 | 30.37 | C |
| ATOM | 1708 | O | HIS | A | 244 | 65.279 | 12.327 | 13.941 | 1.00 | 29.56 | O |
| ATOM | 1709 | N | ASP | A | 245 | 67.326 | 11.360 | 13.689 | 1.00 | 29.19 | N |
| ATOM | 1710 | CA | ASP | A | 245 | 66.909 | 10.373 | 12.691 | 1.00 | 28.54 | C |
| ATOM | 1711 | CB | ASP | A | 245 | 68.005 | 9.315 | 12.473 | 1.00 | 28.34 | C |
| ATOM | 1712 | CG | ASP | A | 245 | 69.208 | 9.853 | 11.726 | 1.00 | 28.71 | C |
| ATOM | 1713 | OD1 | ASP | A | 245 | 69.183 | 11.016 | 11.252 | 1.00 | 28.60 | O |
| ATOM | 1714 | OD2 | ASP | A | 245 | 70.242 | 9.174 | 11.572 | 1.00 | 30.07 | O |
| ATOM | 1715 | C | ASP | A | 245 | 65.624 | 9.670 | 13.103 | 1.00 | 28.04 | C |
| ATOM | 1716 | O | ASP | A | 245 | 64.724 | 9.485 | 12.284 | 1.00 | 27.65 | O |
| ATOM | 1717 | N | GLU | A | 246 | 65.566 | 9.292 | 14.381 | 1.00 | 27.40 | N |
| ATOM | 1718 | CA | GLU | A | 246 | 64.451 | 8.563 | 14.978 | 1.00 | 27.37 | C |
| ATOM | 1719 | CB | GLU | A | 246 | 64.743 | 8.286 | 16.468 | 1.00 | 28.11 | C |
| ATOM | 1720 | CG | GLU | A | 246 | 65.942 | 7.369 | 16.736 | 1.00 | 32.49 | C |
| ATOM | 1721 | CD | GLU | A | 246 | 67.302 | 8.072 | 16.650 | 1.00 | 37.16 | C |
| ATOM | 1722 | OE1 | GLU | A | 246 | 67.413 | 9.255 | 17.037 | 1.00 | 39.44 | O |
| ATOM | 1723 | OE2 | GLU | A | 246 | 68.276 | 7.434 | 16.191 | 1.00 | 40.05 | O |
| ATOM | 1724 | C | GLU | A | 246 | 63.128 | 9.318 | 14.844 | 1.00 | 26.19 | C |
| ATOM | 1725 | O | GLU | A | 246 | 62.087 | 8.720 | 14.570 | 1.00 | 25.74 | O |
| ATOM | 1726 | N | GLU | A | 247 | 63.178 | 10.630 | 15.054 | 1.00 | 24.84 | N |
| ATOM | 1727 | CA | GLU | A | 247 | 61.997 | 11.473 | 14.925 | 1.00 | 24.64 | C |
| ATOM | 1728 | CB | GLU | A | 247 | 62.228 | 12.861 | 15.550 | 1.00 | 24.76 | C |
| ATOM | 1729 | CG | GLU | A | 247 | 62.600 | 12.823 | 17.029 | 1.00 | 27.07 | C |
| ATOM | 1730 | CD | GLU | A | 247 | 63.106 | 14.164 | 17.539 | 1.00 | 31.82 | C |
| ATOM | 1731 | OE1 | GLU | A | 247 | 63.956 | 14.804 | 16.873 | 1.00 | 31.23 | O |
| ATOM | 1732 | OE2 | GLU | A | 247 | 62.653 | 14.582 | 18.623 | 1.00 | 35.62 | O |
| ATOM | 1733 | C | GLU | A | 247 | 61.573 | 11.592 | 13.458 | 1.00 | 23.43 | C |
| ATOM | 1734 | O | GLU | A | 247 | 60.388 | 11.491 | 13.151 | 1.00 | 22.84 | O |
| ATOM | 1735 | N | ILE | A | 248 | 62.546 | 11.782 | 12.563 | 1.00 | 22.82 | N |
| ATOM | 1736 | CA | ILE | A | 248 | 62.269 | 11.827 | 11.119 | 1.00 | 22.09 | C |
| ATOM | 1737 | CB | ILE | A | 248 | 63.555 | 12.106 | 10.284 | 1.00 | 22.33 | C |
| ATOM | 1738 | CG1 | ILE | A | 248 | 64.103 | 13.506 | 10.594 | 1.00 | 21.24 | C |
| ATOM | 1739 | CD1 | ILE | A | 248 | 65.557 | 13.692 | 10.191 | 1.00 | 23.01 | C |
| ATOM | 1740 | CG2 | ILE | A | 248 | 63.273 | 11.965 | 8.767 | 1.00 | 21.11 | C |
| ATOM | 1741 | C | ILE | A | 248 | 61.567 | 10.547 | 10.665 | 1.00 | 22.18 | C |
| ATOM | 1742 | O | ILE | A | 248 | 60.512 | 10.608 | 10.038 | 1.00 | 21.20 | O |
| ATOM | 1743 | N | ILE | A | 249 | 62.144 | 9.396 | 11.016 | 1.00 | 22.55 | N |
| ATOM | 1744 | CA | ILE | A | 249 | 61.595 | 8.083 | 10.646 | 1.00 | 23.21 | C |
| ATOM | 1745 | CB | ILE | A | 249 | 62.539 | 6.943 | 11.136 | 1.00 | 23.49 | C |
| ATOM | 1746 | CG1 | ILE | A | 249 | 63.856 | 6.947 | 10.348 | 1.00 | 24.43 | C |
| ATOM | 1747 | CD1 | ILE | A | 249 | 64.971 | 6.114 | 11.041 | 1.00 | 26.84 | C |
| ATOM | 1748 | CG2 | ILE | A | 249 | 61.853 | 5.562 | 11.051 | 1.00 | 24.03 | C |
| ATOM | 1749 | C | ILE | A | 249 | 60.175 | 7.875 | 11.200 | 1.00 | 23.18 | C |
| ATOM | 1750 | O | ILE | A | 249 | 59.312 | 7.314 | 10.520 | 1.00 | 22.99 | O |
| ATOM | 1751 | N | ARG | A | 250 | 59.943 | 8.318 | 12.435 | 1.00 | 23.13 | N |
| ATOM | 1752 | CA | ARG | A | 250 | 58.614 | 8.204 | 13.044 | 1.00 | 23.60 | C |
| ATOM | 1753 | CB | ARG | A | 250 | 58.679 | 8.421 | 14.562 | 1.00 | 23.56 | C |
| ATOM | 1754 | CG | ARG | A | 250 | 57.356 | 8.162 | 15.290 | 1.00 | 24.55 | C |
| ATOM | 1755 | CD | ARG | A | 250 | 57.504 | 7.770 | 16.760 | 1.00 | 24.93 | C |
| ATOM | 1756 | NE | ARG | A | 250 | 56.208 | 7.638 | 17.430 | 1.00 | 25.45 | N |
| ATOM | 1757 | CZ | ARG | A | 250 | 55.636 | 8.594 | 18.168 | 1.00 | 25.98 | C |
| ATOM | 1758 | NH1 | ARG | A | 250 | 56.234 | 9.770 | 18.349 | 1.00 | 24.37 | N |
| ATOM | 1759 | NH2 | ARG | A | 250 | 54.459 | 8.373 | 18.733 | 1.00 | 26.41 | N |
| ATOM | 1760 | C | ARG | A | 250 | 57.621 | 9.159 | 12.375 | 1.00 | 23.56 | C |
| ATOM | 1761 | O | ARG | A | 250 | 56.468 | 8.802 | 12.165 | 1.00 | 23.53 | O |
| ATOM | 1762 | N | GLY | A | 251 | 58.089 | 10.357 | 12.022 | 1.00 | 23.63 | N |
| ATOM | 1763 | CA | GLY | A | 251 | 57.282 | 11.334 | 11.314 | 1.00 | 24.56 | C |
| ATOM | 1764 | C | GLY | A | 251 | 56.082 | 11.864 | 12.096 | 1.00 | 25.30 | C |
| ATOM | 1765 | O | GLY | A | 251 | 55.074 | 12.248 | 11.496 | 1.00 | 25.76 | O |
| ATOM | 1766 | N | GLN | A | 252 | 56.177 | 11.877 | 13.423 | 1.00 | 25.26 | N |
| ATOM | 1767 | CA | GLN | A | 252 | 55.082 | 12.373 | 14.263 | 1.00 | 25.84 | C |
| ATOM | 1768 | CB | GLN | A | 252 | 55.005 | 11.603 | 15.593 | 1.00 | 26.06 | C |
| ATOM | 1769 | CG | GLN | A | 252 | 53.796 | 11.937 | 16.488 | 1.00 | 29.12 | C |
| ATOM | 1770 | CD | GLN | A | 252 | 52.439 | 11.649 | 15.837 | 1.00 | 32.13 | C |
| ATOM | 1771 | OE1 | GLN | A | 252 | 51.537 | 12.506 | 15.854 | 1.00 | 31.35 | O |
| ATOM | 1772 | NE2 | GLN | A | 252 | 52.292 | 10.448 | 15.264 | 1.00 | 32.42 | N |
| ATOM | 1773 | C | GLN | A | 252 | 55.271 | 13.863 | 14.504 | 1.00 | 25.11 | C |
| ATOM | 1774 | O | GLN | A | 252 | 56.310 | 14.303 | 15.008 | 1.00 | 24.98 | O |
| ATOM | 1775 | N | VAL | A | 253 | 54.265 | 14.634 | 14.123 | 1.00 | 24.33 | N |
| ATOM | 1776 | CA | VAL | A | 253 | 54.351 | 16.085 | 14.196 | 1.00 | 24.36 | C |
| ATOM | 1777 | CB | VAL | A | 253 | 53.751 | 16.750 | 12.922 | 1.00 | 24.24 | C |
| ATOM | 1778 | CG1 | VAL | A | 253 | 53.971 | 18.240 | 12.948 | 1.00 | 24.73 | C |
| ATOM | 1779 | CG2 | VAL | A | 253 | 54.356 | 16.124 | 11.647 | 1.00 | 24.87 | C |
| ATOM | 1780 | C | VAL | A | 253 | 53.601 | 16.576 | 15.431 | 1.00 | 23.69 | C |
| ATOM | 1781 | O | VAL | A | 253 | 52.427 | 16.275 | 15.602 | 1.00 | 23.22 | O |
| ATOM | 1782 | N | PHE | A | 254 | 54.297 | 17.321 | 16.278 | 1.00 | 23.14 | N |
| ATOM | 1783 | CA | PHE | A | 254 | 53.683 | 17.993 | 17.403 | 1.00 | 23.19 | C |
| ATOM | 1784 | CB | PHE | A | 254 | 54.295 | 17.481 | 18.702 | 1.00 | 22.64 | C |
| ATOM | 1785 | CG | APHE | A | 254 | 53.868 | 18.247 | 19.912 | 0.70 | 21.91 | C |
| ATOM | 1786 | CG | BPHE | A | 254 | 53.915 | 16.067 | 18.997 | 0.30 | 22.47 | C |
| ATOM | 1787 | CD1 | APHE | A | 254 | 52.711 | 17.897 | 20.596 | 0.70 | 20.57 | C |
| ATOM | 1788 | CD1 | BPHE | A | 254 | 54.877 | 15.073 | 19.055 | 0.30 | 21.39 | C |
| ATOM | 1789 | CE1 | APHE | A | 254 | 52.308 | 18.608 | 21.724 | 0.70 | 18.82 | C |
| ATOM | 1790 | CE1 | BPHE | A | 254 | 54.517 | 13.772 | 19.304 | 0.30 | 20.97 | C |
| ATOM | 1791 | CZ | APHE | A | 254 | 53.054 | 19.677 | 22.163 | 0.70 | 20.33 | C |
| ATOM | 1792 | CZ | BPHE | A | 254 | 53.180 | 13.445 | 19.476 | 0.30 | 21.52 | C |
| ATOM | 1793 | CE2 | APHE | A | 254 | 54.214 | 20.045 | 21.486 | 0.70 | 20.58 | C |
| ATOM | 1794 | CE2 | BPHE | A | 254 | 52.207 | 14.421 | 19.400 | 0.30 | 21.67 | C |
| ATOM | 1795 | CD2 | APHE | A | 254 | 54.615 | 19.333 | 20.369 | 0.70 | 21.56 | C |
| ATOM | 1796 | CD2 | BPHE | A | 254 | 52.574 | 15.720 | 19.157 | 0.30 | 21.61 | C |
| ATOM | 1797 | C | PHE | A | 254 | 53.789 | 19.507 | 17.295 | 1.00 | 23.65 | C |
| ATOM | 1798 | O | PHE | A | 254 | 54.876 | 20.055 | 17.059 | 1.00 | 23.03 | O |
| ATOM | 1799 | N | PHE | A | 255 | 52.652 | 20.173 | 17.475 | 1.00 | 24.25 | N |
| ATOM | 1800 | CA | PHE | A | 255 | 52.600 | 21.636 | 17.448 | 1.00 | 24.57 | C |
| ATOM | 1801 | CB | PHE | A | 255 | 51.367 | 22.117 | 16.705 | 1.00 | 24.07 | C |
| ATOM | 1802 | CG | PHE | A | 255 | 51.421 | 21.819 | 15.250 | 1.00 | 22.93 | C |
| ATOM | 1803 | CD1 | PHE | A | 255 | 51.972 | 22.743 | 14.368 | 1.00 | 20.88 | C |
| ATOM | 1804 | CE1 | PHE | A | 255 | 52.048 | 22.466 | 13.018 | 1.00 | 18.91 | C |
| ATOM | 1805 | CZ | PHE | A | 255 | 51.585 | 21.254 | 12.540 | 1.00 | 20.61 | C |
| ATOM | 1806 | CE2 | PHE | A | 255 | 51.047 | 20.307 | 13.426 | 1.00 | 19.19 | C |
| ATOM | 1807 | CD2 | PHE | A | 255 | 50.974 | 20.595 | 14.762 | 1.00 | 19.54 | C |
| ATOM | 1808 | C | PHE | A | 255 | 52.668 | 22.239 | 18.830 | 1.00 | 25.43 | C |
| ATOM | 1809 | O | PHE | A | 255 | 51.839 | 21.958 | 19.691 | 1.00 | 25.30 | O |
| ATOM | 1810 | N | ARG | A | 256 | 53.701 | 23.057 | 19.015 | 1.00 | 26.64 | N |
| ATOM | 1811 | CA | ARG | A | 256 | 54.026 | 23.713 | 20.274 | 1.00 | 27.50 | C |
| ATOM | 1812 | CB | ARG | A | 256 | 55.556 | 23.791 | 20.412 | 1.00 | 28.35 | C |
| ATOM | 1813 | CG | ARG | A | 256 | 56.282 | 24.268 | 19.116 | 1.00 | 31.19 | C |
| ATOM | 1814 | CD | ARG | A | 256 | 57.825 | 24.203 | 19.164 | 1.00 | 35.66 | C |
| ATOM | 1815 | NE | ARG | A | 256 | 58.346 | 23.171 | 18.257 | 1.00 | 38.13 | N |
| ATOM | 1816 | CZ | ARG | A | 256 | 59.580 | 22.660 | 18.298 | 1.00 | 40.12 | C |
| ATOM | 1817 | NH1 | ARG | A | 256 | 60.466 | 23.080 | 19.204 | 1.00 | 40.47 | N |
| ATOM | 1818 | NH2 | ARG | A | 256 | 59.930 | 21.716 | 17.431 | 1.00 | 38.85 | N |
| ATOM | 1819 | C | ARG | A | 256 | 53.444 | 25.122 | 20.255 | 1.00 | 27.08 | C |
| ATOM | 1820 | O | ARG | A | 256 | 53.389 | 25.807 | 21.279 | 1.00 | 27.90 | O |
| ATOM | 1821 | N | GLN | A | 257 | 53.023 | 25.546 | 19.068 | 1.00 | 26.07 | N |
| ATOM | 1822 | CA | GLN | A | 257 | 52.369 | 26.834 | 18.871 | 1.00 | 25.36 | C |
| ATOM | 1823 | CB | GLN | A | 257 | 53.126 | 27.643 | 17.803 | 1.00 | 25.81 | C |
| ATOM | 1824 | CG | GLN | A | 257 | 54.514 | 28.095 | 18.215 | 1.00 | 29.91 | C |
| ATOM | 1825 | CD | GLN | A | 257 | 54.493 | 29.381 | 19.019 | 1.00 | 35.63 | C |
| ATOM | 1826 | OE1 | GLN | A | 257 | 53.596 | 30.222 | 18.846 | 1.00 | 37.93 | O |
| ATOM | 1827 | NE2 | GLN | A | 257 | 55.480 | 29.545 | 19.901 | 1.00 | 37.58 | N |
| ATOM | 1828 | C | GLN | A | 257 | 50.931 | 26.611 | 18.399 | 1.00 | 23.15 | C |
| ATOM | 1829 | O | GLN | A | 257 | 50.618 | 25.574 | 17.821 | 1.00 | 22.57 | O |
| ATOM | 1830 | N | ARG | A | 258 | 50.072 | 27.593 | 18.633 | 1.00 | 21.04 | N |
| ATOM | 1831 | CA | ARG | A | 258 | 48.726 | 27.571 | 18.079 | 1.00 | 19.58 | C |
| ATOM | 1832 | CB | ARG | A | 258 | 47.862 | 28.674 | 18.683 | 1.00 | 19.69 | C |
| ATOM | 1833 | CG | ARG | A | 258 | 46.355 | 28.438 | 18.509 | 1.00 | 21.79 | C |
| ATOM | 1834 | CD | ARG | A | 258 | 45.834 | 28.809 | 17.134 | 1.00 | 24.81 | C |
| ATOM | 1835 | NE | ARG | A | 258 | 44.538 | 28.195 | 16.847 | 1.00 | 26.49 | N |
| ATOM | 1836 | CZ | ARG | A | 258 | 43.844 | 28.395 | 15.725 | 1.00 | 27.04 | C |
| ATOM | 1837 | NH1 | ARG | A | 258 | 44.316 | 29.200 | 14.757 | 1.00 | 27.33 | N |
| ATOM | 1838 | NH2 | ARG | A | 258 | 42.677 | 27.789 | 15.570 | 1.00 | 25.64 | N |
| ATOM | 1839 | C | ARG | A | 258 | 48.811 | 27.738 | 16.564 | 1.00 | 18.78 | C |
| ATOM | 1840 | O | ARG | A | 258 | 49.282 | 28.759 | 16.074 | 1.00 | 18.29 | O |
| ATOM | 1841 | N | VAL | A | 259 | 48.367 | 26.716 | 15.843 | 1.00 | 17.49 | N |
| ATOM | 1842 | CA | VAL | A | 259 | 48.404 | 26.682 | 14.389 | 1.00 | 17.02 | C |
| ATOM | 1843 | CB | VAL | A | 259 | 49.533 | 25.733 | 13.879 | 1.00 | 16.57 | C |
| ATOM | 1844 | CG1 | VAL | A | 259 | 49.472 | 25.544 | 12.361 | 1.00 | 15.19 | C |
| ATOM | 1845 | CG2 | VAL | A | 259 | 50.929 | 26.259 | 14.310 | 1.00 | 17.98 | C |
| ATOM | 1846 | C | VAL | A | 259 | 47.043 | 26.171 | 13.920 | 1.00 | 16.99 | C |
| ATOM | 1847 | O | VAL | A | 259 | 46.541 | 25.190 | 14.460 | 1.00 | 16.43 | O |
| ATOM | 1848 | N | SER | A | 260 | 46.451 | 26.843 | 12.930 | 1.00 | 17.02 | N |
| ATOM | 1849 | CA | SER | A | 260 | 45.141 | 26.451 | 12.398 | 1.00 | 17.31 | C |
| ATOM | 1850 | CB | SER | A | 260 | 44.687 | 27.398 | 11.273 | 1.00 | 17.29 | C |
| ATOM | 1851 | OG | SER | A | 260 | 45.399 | 27.137 | 10.073 | 1.00 | 17.50 | O |
| ATOM | 1852 | C | SER | A | 260 | 45.145 | 25.005 | 11.916 | 1.00 | 17.62 | C |
| ATOM | 1853 | O | SER | A | 260 | 46.182 | 24.484 | 11.518 | 1.00 | 17.40 | O |
| ATOM | 1854 | N | SER | A | 261 | 43.974 | 24.367 | 11.957 | 1.00 | 17.98 | N |
| ATOM | 1855 | CA | SER | A | 261 | 43.820 | 22.972 | 11.559 | 1.00 | 18.43 | C |
| ATOM | 1856 | CB | SER | A | 261 | 42.398 | 22.499 | 11.855 | 1.00 | 18.46 | C |
| ATOM | 1857 | OG | SER | A | 261 | 42.169 | 22.473 | 13.254 | 1.00 | 19.59 | O |
| ATOM | 1858 | C | SER | A | 261 | 44.118 | 22.748 | 10.082 | 1.00 | 18.64 | C |
| ATOM | 1859 | O | SER | A | 261 | 44.630 | 21.694 | 9.701 | 1.00 | 18.31 | O |
| ATOM | 1860 | N | GLU | A | 262 | 43.780 | 23.729 | 9.256 | 1.00 | 19.27 | N |
| ATOM | 1861 | CA | GLU | A | 262 | 44.102 | 23.659 | 7.829 | 1.00 | 20.49 | C |
| ATOM | 1862 | CB | GLU | A | 262 | 43.461 | 24.807 | 7.058 | 1.00 | 21.45 | C |
| ATOM | 1863 | CG | GLU | A | 262 | 42.033 | 24.525 | 6.627 | 1.00 | 27.18 | C |
| ATOM | 1864 | CD | GLU | A | 262 | 41.304 | 25.782 | 6.184 | 1.00 | 35.25 | C |
| ATOM | 1865 | OE1 | GLU | A | 262 | 41.928 | 26.645 | 5.498 | 1.00 | 38.82 | O |
| ATOM | 1866 | OE2 | GLU | A | 262 | 40.101 | 25.915 | 6.522 | 1.00 | 39.29 | O |
| ATOM | 1867 | C | GLU | A | 262 | 45.615 | 23.663 | 7.614 | 1.00 | 19.31 | C |
| ATOM | 1868 | O | GLU | A | 262 | 46.131 | 22.851 | 6.853 | 1.00 | 18.98 | O |
| ATOM | 1869 | N | CYS | A | 263 | 46.318 | 24.561 | 8.297 | 1.00 | 18.97 | N |
| ATOM | 1870 | CA | CYS | A | 263 | 47.785 | 24.596 | 8.196 | 1.00 | 18.66 | C |
| ATOM | 1871 | CB | CYS | A | 263 | 48.359 | 25.805 | 8.937 | 1.00 | 18.56 | C |
| ATOM | 1872 | SG | CYS | A | 263 | 50.133 | 26.031 | 8.731 | 1.00 | 18.69 | S |
| ATOM | 1873 | C | CYS | A | 263 | 48.385 | 23.275 | 8.703 | 1.00 | 18.45 | C |
| ATOM | 1874 | O | CYS | A | 263 | 49.223 | 22.664 | 8.024 | 1.00 | 18.06 | O |
| ATOM | 1875 | N | GLN | A | 264 | 47.932 | 22.827 | 9.873 | 1.00 | 18.01 | N |
| ATOM | 1876 | CA | GLN | A | 264 | 48.389 | 21.553 | 10.434 | 1.00 | 18.32 | C |
| ATOM | 1877 | CB | GLN | A | 264 | 47.650 | 21.210 | 11.748 | 1.00 | 17.97 | C |
| ATOM | 1878 | CG | GLN | A | 264 | 48.085 | 22.034 | 12.955 | 1.00 | 18.25 | C |
| ATOM | 1879 | CD | GLN | A | 264 | 47.598 | 21.447 | 14.282 | 1.00 | 20.77 | C |
| ATOM | 1880 | OE1 | GLN | A | 264 | 47.359 | 20.240 | 14.382 | 1.00 | 19.45 | O |
| ATOM | 1881 | NE2 | GLN | A | 264 | 47.464 | 22.299 | 15.304 | 1.00 | 19.10 | N |
| ATOM | 1882 | C | GLN | A | 264 | 48.191 | 20.419 | 9.424 | 1.00 | 18.18 | C |
| ATOM | 1883 | O | GLN | A | 264 | 49.068 | 19.581 | 9.252 | 1.00 | 18.03 | O |
| ATOM | 1884 | N | HIS | A | 265 | 47.033 | 20.405 | 8.768 | 1.00 | 18.36 | N |
| ATOM | 1885 | CA | HIS | A | 265 | 46.712 | 19.366 | 7.805 | 1.00 | 19.15 | C |
| ATOM | 1886 | CB | HIS | A | 265 | 45.269 | 19.505 | 7.310 | 1.00 | 19.80 | C |
| ATOM | 1887 | CG | HIS | A | 265 | 44.890 | 18.474 | 6.295 | 1.00 | 23.71 | C |
| ATOM | 1888 | ND1 | HIS | A | 265 | 45.147 | 18.627 | 4.948 | 1.00 | 27.52 | N |
| ATOM | 1889 | CE1 | HIS | A | 265 | 44.712 | 17.562 | 4.294 | 1.00 | 28.98 | C |
| ATOM | 1890 | NE2 | HIS | A | 265 | 44.190 | 16.720 | 5.170 | 1.00 | 29.95 | N |
| ATOM | 1891 | CD2 | HIS | A | 265 | 44.294 | 17.264 | 6.430 | 1.00 | 27.50 | C |
| ATOM | 1892 | C | HIS | A | 265 | 47.701 | 19.383 | 6.624 | 1.00 | 18.47 | C |
| ATOM | 1893 | O | HIS | A | 265 | 48.219 | 18.340 | 6.236 | 1.00 | 17.33 | O |
| ATOM | 1894 | N | LEU | A | 266 | 47.973 | 20.577 | 6.089 | 1.00 | 17.86 | N |
| ATOM | 1895 | CA | LEU | A | 266 | 48.891 | 20.717 | 4.968 | 1.00 | 17.70 | C |
| ATOM | 1896 | CB | LEU | A | 266 | 48.925 | 22.167 | 4.440 | 1.00 | 17.79 | C |
| ATOM | 1897 | CG | LEU | A | 266 | 49.889 | 22.490 | 3.277 | 1.00 | 16.98 | C |
| ATOM | 1898 | CD1 | LEU | A | 266 | 49.700 | 21.533 | 2.080 | 1.00 | 16.25 | C |
| ATOM | 1899 | CD2 | LEU | A | 266 | 49.731 | 23.941 | 2.832 | 1.00 | 16.33 | C |
| ATOM | 1900 | C | LEU | A | 266 | 50.282 | 20.225 | 5.372 | 1.00 | 17.61 | C |
| ATOM | 1901 | O | LEU | A | 266 | 50.906 | 19.443 | 4.642 | 1.00 | 17.30 | O |
| ATOM | 1902 | N | ILE | A | 267 | 50.741 | 20.639 | 6.555 | 1.00 | 17.19 | N |
| ATOM | 1903 | CA | ILE | A | 267 | 52.072 | 20.263 | 7.023 | 1.00 | 16.80 | C |
| ATOM | 1904 | CB | ILE | A | 267 | 52.425 | 20.934 | 8.385 | 1.00 | 16.67 | C |
| ATOM | 1905 | CG1 | ILE | A | 267 | 52.702 | 22.433 | 8.196 | 1.00 | 15.36 | C |
| ATOM | 1906 | CD1 | ILE | A | 267 | 52.656 | 23.273 | 9.494 | 1.00 | 14.12 | C |
| ATOM | 1907 | CG2 | ILE | A | 267 | 53.626 | 20.245 | 9.024 | 1.00 | 15.66 | C |
| ATOM | 1908 | C | ILE | A | 267 | 52.173 | 18.753 | 7.137 | 1.00 | 17.49 | C |
| ATOM | 1909 | O | ILE | A | 267 | 53.119 | 18.156 | 6.618 | 1.00 | 16.97 | O |
| ATOM | 1910 | N | ARG | A | 268 | 51.178 | 18.140 | 7.783 | 1.00 | 17.52 | N |
| ATOM | 1911 | CA | ARG | A | 268 | 51.165 | 16.692 | 7.997 | 1.00 | 18.02 | C |
| ATOM | 1912 | CB | ARG | A | 268 | 49.990 | 16.302 | 8.907 | 1.00 | 18.56 | C |
| ATOM | 1913 | CG | ARG | A | 268 | 50.240 | 16.587 | 10.386 | 1.00 | 20.63 | C |
| ATOM | 1914 | CD | ARG | A | 268 | 49.234 | 15.899 | 11.331 | 1.00 | 25.57 | C |
| ATOM | 1915 | NE | ARG | A | 268 | 48.912 | 16.743 | 12.487 | 1.00 | 29.85 | N |
| ATOM | 1916 | CZ | ARG | A | 268 | 49.629 | 16.737 | 13.585 | 1.00 | 31.14 | C |
| ATOM | 1917 | NH1 | ARG | A | 268 | 50.663 | 15.929 | 13.648 | 1.00 | 34.34 | N |
| ATOM | 1918 | NH2 | ARG | A | 268 | 49.331 | 17.507 | 14.615 | 1.00 | 30.34 | N |
| ATOM | 1919 | C | ARG | A | 268 | 51.104 | 15.910 | 6.668 | 1.00 | 17.59 | C |
| ATOM | 1920 | O | ARG | A | 268 | 51.676 | 14.833 | 6.544 | 1.00 | 16.65 | O |
| ATOM | 1921 | N | TRP | A | 269 | 50.397 | 16.470 | 5.693 | 1.00 | 17.56 | N |
| ATOM | 1922 | CA | TRP | A | 269 | 50.336 | 15.913 | 4.341 | 1.00 | 18.04 | C |
| ATOM | 1923 | CB | TRP | A | 269 | 49.340 | 16.717 | 3.490 | 1.00 | 18.77 | C |
| ATOM | 1924 | CG | TRP | A | 269 | 48.810 | 15.979 | 2.265 | 1.00 | 21.08 | C |
| ATOM | 1925 | CD1 | TRP | A | 269 | 49.030 | 14.662 | 1.914 | 1.00 | 22.56 | C |
| ATOM | 1926 | NE1 | TRP | A | 269 | 48.387 | 14.372 | 0.730 | 1.00 | 24.35 | N |
| ATOM | 1927 | CE2 | TRP | A | 269 | 47.716 | 15.491 | 0.301 | 1.00 | 23.34 | C |
| ATOM | 1928 | CD2 | TRP | A | 269 | 47.957 | 16.522 | 1.248 | 1.00 | 22.70 | C |
| ATOM | 1929 | CE3 | TRP | A | 269 | 47.377 | 17.781 | 1.033 | 1.00 | 23.10 | C |
| ATOM | 1930 | CZ3 | TRP | A | 269 | 46.576 | 17.970 | −0.102 | 1.00 | 24.93 | C |
| ATOM | 1931 | CH2 | TRP | A | 269 | 46.351 | 16.922 | −1.014 | 1.00 | 24.84 | C |
| ATOM | 1932 | CZ2 | TRP | A | 269 | 46.911 | 15.679 | −0.829 | 1.00 | 23.75 | C |
| ATOM | 1933 | C | TRP | A | 269 | 51.711 | 15.906 | 3.667 | 1.00 | 17.27 | C |
| ATOM | 1934 | O | TRP | A | 269 | 52.137 | 14.870 | 3.149 | 1.00 | 16.99 | O |
| ATOM | 1935 | N | CYS | A | 270 | 52.400 | 17.056 | 3.687 | 1.00 | 16.34 | N |
| ATOM | 1936 | CA | CYS | A | 270 | 53.759 | 17.173 | 3.134 | 1.00 | 16.21 | C |
| ATOM | 1937 | CB | CYS | A | 270 | 54.294 | 18.607 | 3.275 | 1.00 | 15.97 | C |
| ATOM | 1938 | SG | CYS | A | 270 | 53.427 | 19.842 | 2.287 | 1.00 | 16.89 | S |
| ATOM | 1939 | C | CYS | A | 270 | 54.742 | 16.241 | 3.824 | 1.00 | 16.04 | C |
| ATOM | 1940 | O | CYS | A | 270 | 55.711 | 15.774 | 3.195 | 1.00 | 15.33 | O |
| ATOM | 1941 | N | LEU | A | 271 | 54.488 | 15.978 | 5.112 | 1.00 | 15.59 | N |
| ATOM | 1942 | CA | LEU | A | 271 | 55.357 | 15.124 | 5.907 | 1.00 | 16.36 | C |
| ATOM | 1943 | CB | LEU | A | 271 | 55.574 | 15.727 | 7.304 | 1.00 | 15.90 | C |
| ATOM | 1944 | CG | LEU | A | 271 | 56.248 | 17.116 | 7.361 | 1.00 | 16.31 | C |
| ATOM | 1945 | CD1 | LEU | A | 271 | 56.473 | 17.592 | 8.793 | 1.00 | 13.91 | C |
| ATOM | 1946 | CD2 | LEU | A | 271 | 57.590 | 17.113 | 6.570 | 1.00 | 14.64 | C |
| ATOM | 1947 | C | LEU | A | 271 | 54.861 | 13.667 | 6.010 | 1.00 | 16.92 | C |
| ATOM | 1948 | O | LEU | A | 271 | 55.190 | 12.969 | 6.976 | 1.00 | 17.11 | O |
| ATOM | 1949 | N | ALA | A | 272 | 54.085 | 13.217 | 5.021 | 1.00 | 17.22 | N |
| ATOM | 1950 | CA | ALA | A | 272 | 53.627 | 11.819 | 4.971 | 1.00 | 18.16 | C |
| ATOM | 1951 | CB | ALA | A | 272 | 52.691 | 11.581 | 3.798 | 1.00 | 18.05 | C |
| ATOM | 1952 | C | ALA | A | 272 | 54.839 | 10.921 | 4.852 | 1.00 | 18.59 | C |
| ATOM | 1953 | O | ALA | A | 272 | 55.768 | 11.216 | 4.083 | 1.00 | 18.17 | O |
| ATOM | 1954 | N | LEU | A | 273 | 54.835 | 9.835 | 5.621 | 1.00 | 19.19 | N |
| ATOM | 1955 | CA | LEU | A | 273 | 55.953 | 8.894 | 5.630 | 1.00 | 19.94 | C |
| ATOM | 1956 | CB | LEU | A | 273 | 55.754 | 7.832 | 6.728 | 1.00 | 20.56 | C |
| ATOM | 1957 | CG | LEU | A | 273 | 56.079 | 8.298 | 8.162 | 1.00 | 21.18 | C |
| ATOM | 1958 | CD1 | LEU | A | 273 | 55.894 | 7.176 | 9.193 | 1.00 | 21.79 | C |
| ATOM | 1959 | CD2 | LEU | A | 273 | 57.491 | 8.889 | 8.262 | 1.00 | 20.34 | C |
| ATOM | 1960 | C | LEU | A | 273 | 56.178 | 8.254 | 4.258 | 1.00 | 20.58 | C |
| ATOM | 1961 | O | LEU | A | 273 | 57.322 | 8.138 | 3.800 | 1.00 | 20.82 | O |
| ATOM | 1962 | N | ARG | A | 274 | 55.090 | 7.849 | 3.605 | 1.00 | 20.78 | N |
| ATOM | 1963 | CA | ARG | A | 274 | 55.164 | 7.292 | 2.259 | 1.00 | 21.86 | C |
| ATOM | 1964 | CB | ARG | A | 274 | 53.968 | 6.373 | 1.955 | 1.00 | 22.23 | C |
| ATOM | 1965 | CG | ARG | A | 274 | 53.714 | 5.266 | 2.975 | 1.00 | 27.91 | C |
| ATOM | 1966 | CD | ARG | A | 274 | 52.588 | 4.263 | 2.576 | 1.00 | 35.10 | C |
| ATOM | 1967 | NE | ARG | A | 274 | 52.637 | 3.917 | 1.150 | 1.00 | 40.44 | N |
| ATOM | 1968 | CZ | ARG | A | 274 | 51.914 | 2.962 | 0.564 | 1.00 | 44.04 | C |
| ATOM | 1969 | NH1 | ARG | A | 274 | 51.061 | 2.223 | 1.275 | 1.00 | 45.20 | N |
| ATOM | 1970 | NH2 | ARG | A | 274 | 52.047 | 2.741 | −0.742 | 1.00 | 44.81 | N |
| ATOM | 1971 | C | ARG | A | 274 | 55.226 | 8.418 | 1.227 | 1.00 | 20.85 | C |
| ATOM | 1972 | O | ARG | A | 274 | 54.312 | 9.249 | 1.157 | 1.00 | 20.77 | O |
| ATOM | 1973 | N | PRO | A | 275 | 56.297 | 8.452 | 0.435 | 1.00 | 20.06 | N |
| ATOM | 1974 | CA | PRO | A | 275 | 56.479 | 9.502 | −0.576 | 1.00 | 20.15 | C |
| ATOM | 1975 | CB | PRO | A | 275 | 57.684 | 9.007 | −1.384 | 1.00 | 19.41 | C |
| ATOM | 1976 | CG | PRO | A | 275 | 58.448 | 8.169 | −0.409 | 1.00 | 19.87 | C |
| ATOM | 1977 | CD | PRO | A | 275 | 57.432 | 7.509 | 0.469 | 1.00 | 20.03 | C |
| ATOM | 1978 | C | PRO | A | 275 | 55.245 | 9.709 | −1.474 | 1.00 | 20.50 | C |
| ATOM | 1979 | O | PRO | A | 275 | 54.842 | 10.860 | −1.692 | 1.00 | 20.44 | O |
| ATOM | 1980 | N | SER | A | 276 | 54.650 | 8.618 | −1.966 | 1.00 | 20.58 | N |
| ATOM | 1981 | CA | SER | A | 276 | 53.454 | 8.693 | −2.811 | 1.00 | 20.56 | C |
| ATOM | 1982 | CB | SER | A | 276 | 53.146 | 7.323 | −3.430 | 1.00 | 20.69 | C |
| ATOM | 1983 | OG | SER | A | 276 | 52.516 | 6.487 | −2.479 | 1.00 | 22.25 | O |
| ATOM | 1984 | C | SER | A | 276 | 52.219 | 9.234 | −2.067 | 1.00 | 20.21 | C |
| ATOM | 1985 | O | SER | A | 276 | 51.232 | 9.612 | −2.697 | 1.00 | 20.43 | O |
| ATOM | 1986 | N | ASP | A | 277 | 52.264 | 9.266 | −0.737 | 1.00 | 19.88 | N |
| ATOM | 1987 | CA | ASP | A | 277 | 51.173 | 9.875 | 0.027 | 1.00 | 19.72 | C |
| ATOM | 1988 | CB | ASP | A | 277 | 51.093 | 9.311 | 1.443 | 1.00 | 19.57 | C |
| ATOM | 1989 | CG | ASP | A | 277 | 50.404 | 7.945 | 1.501 | 1.00 | 21.58 | C |
| ATOM | 1990 | OD1 | ASP | A | 277 | 49.751 | 7.540 | 0.504 | 1.00 | 20.40 | O |
| ATOM | 1991 | OD2 | ASP | A | 277 | 50.470 | 7.222 | 2.522 | 1.00 | 21.84 | O |
| ATOM | 1992 | C | ASP | A | 277 | 51.266 | 11.407 | 0.085 | 1.00 | 19.39 | C |
| ATOM | 1993 | O | ASP | A | 277 | 50.295 | 12.068 | 0.483 | 1.00 | 20.39 | O |
| ATOM | 1994 | N | ARG | A | 278 | 52.413 | 11.962 | −0.308 | 1.00 | 18.14 | N |
| ATOM | 1995 | CA | ARG | A | 278 | 52.633 | 13.408 | −0.252 | 1.00 | 17.60 | C |
| ATOM | 1996 | CB | ARG | A | 278 | 54.137 | 13.740 | −0.277 | 1.00 | 17.19 | C |
| ATOM | 1997 | CG | ARG | A | 278 | 54.859 | 13.330 | 1.009 | 1.00 | 16.29 | C |
| ATOM | 1998 | CD | ARG | A | 278 | 56.388 | 13.456 | 0.995 | 1.00 | 15.61 | C |
| ATOM | 1999 | NE | ARG | A | 278 | 56.954 | 12.458 | 1.908 | 1.00 | 15.33 | N |
| ATOM | 2000 | CZ | ARG | A | 278 | 58.152 | 11.885 | 1.769 | 1.00 | 15.76 | C |
| ATOM | 2001 | NH1 | ARG | A | 278 | 58.965 | 12.239 | 0.770 | 1.00 | 13.75 | N |
| ATOM | 2002 | NH2 | ARG | A | 278 | 58.541 | 10.966 | 2.649 | 1.00 | 13.90 | N |
| ATOM | 2003 | C | ARG | A | 278 | 51.908 | 14.104 | −1.391 | 1.00 | 17.58 | C |
| ATOM | 2004 | O | ARG | A | 278 | 51.716 | 13.506 | −2.466 | 1.00 | 17.77 | O |
| ATOM | 2005 | N | PRO | A | 279 | 51.508 | 15.357 | −1.166 | 1.00 | 16.97 | N |
| ATOM | 2006 | CA | PRO | A | 279 | 50.820 | 16.142 | −2.192 | 1.00 | 16.86 | C |
| ATOM | 2007 | CB | PRO | A | 279 | 50.364 | 17.387 | −1.415 | 1.00 | 17.33 | C |
| ATOM | 2008 | CG | PRO | A | 279 | 51.426 | 17.533 | −0.313 | 1.00 | 16.46 | C |
| ATOM | 2009 | CD | PRO | A | 279 | 51.685 | 16.125 | 0.088 | 1.00 | 16.42 | C |
| ATOM | 2010 | C | PRO | A | 279 | 51.768 | 16.573 | −3.290 | 1.00 | 17.64 | C |
| ATOM | 2011 | O | PRO | A | 279 | 52.967 | 16.757 | −3.021 | 1.00 | 17.91 | O |
| ATOM | 2012 | N | THR | A | 280 | 51.245 | 16.735 | −4.507 | 1.00 | 17.35 | N |
| ATOM | 2013 | CA | THR | A | 280 | 51.998 | 17.353 | −5.593 | 1.00 | 17.56 | C |
| ATOM | 2014 | CB | THR | A | 280 | 51.284 | 17.108 | −6.937 | 1.00 | 17.70 | C |
| ATOM | 2015 | OG1 | THR | A | 280 | 49.989 | 17.716 | −6.885 | 1.00 | 18.13 | O |
| ATOM | 2016 | CG2 | THR | A | 280 | 50.976 | 15.600 | −7.152 | 1.00 | 18.27 | C |
| ATOM | 2017 | C | THR | A | 280 | 52.048 | 18.864 | −5.326 | 1.00 | 18.09 | C |
| ATOM | 2018 | O | THR | A | 280 | 51.342 | 19.358 | −4.427 | 1.00 | 18.12 | O |
| ATOM | 2019 | N | PHE | A | 281 | 52.838 | 19.600 | −6.113 | 1.00 | 18.50 | N |
| ATOM | 2020 | CA | PHE | A | 281 | 52.870 | 21.066 | −6.000 | 1.00 | 19.56 | C |
| ATOM | 2021 | CB | PHE | A | 281 | 53.863 | 21.702 | −6.994 | 1.00 | 19.97 | C |
| ATOM | 2022 | CG | PHE | A | 281 | 55.322 | 21.369 | −6.721 | 1.00 | 22.52 | C |
| ATOM | 2023 | CD1 | PHE | A | 281 | 55.834 | 21.383 | −5.433 | 1.00 | 25.27 | C |
| ATOM | 2024 | CE1 | PHE | A | 281 | 57.198 | 21.070 | −5.177 | 1.00 | 26.96 | C |
| ATOM | 2025 | CZ | PHE | A | 281 | 58.040 | 20.748 | −6.235 | 1.00 | 29.27 | C |
| ATOM | 2026 | CE2 | PHE | A | 281 | 57.535 | 20.748 | −7.553 | 1.00 | 28.17 | C |
| ATOM | 2027 | CD2 | PHE | A | 281 | 56.183 | 21.061 | −7.781 | 1.00 | 26.44 | C |
| ATOM | 2028 | C | PHE | A | 281 | 51.474 | 21.656 | −6.211 | 1.00 | 19.44 | C |
| ATOM | 2029 | O | PHE | A | 281 | 51.064 | 22.559 | −5.481 | 1.00 | 19.75 | O |
| ATOM | 2030 | N | GLU | A | 282 | 50.742 | 21.119 | −7.188 | 1.00 | 18.94 | N |
| ATOM | 2031 | CA | GLU | A | 282 | 49.396 | 21.592 | −7.492 | 1.00 | 19.12 | C |
| ATOM | 2032 | CB | GLU | A | 282 | 48.851 | 20.940 | −8.787 | 1.00 | 19.45 | C |
| ATOM | 2033 | CG | GLU | A | 282 | 47.360 | 21.133 | −9.034 | 1.00 | 21.47 | C |
| ATOM | 2034 | CD | GLU | A | 282 | 46.874 | 20.578 | −10.387 | 1.00 | 26.22 | C |
| ATOM | 2035 | OE1 | GLU | A | 282 | 47.449 | 19.584 | −10.886 | 1.00 | 26.51 | O |
| ATOM | 2036 | OE2 | GLU | A | 282 | 45.901 | 21.136 | −10.951 | 1.00 | 25.95 | O |
| ATOM | 2037 | C | GLU | A | 282 | 48.454 | 21.369 | −6.307 | 1.00 | 18.61 | C |
| ATOM | 2038 | O | GLU | A | 282 | 47.648 | 22.248 | −5.986 | 1.00 | 18.93 | O |
| ATOM | 2039 | N | GLU | A | 283 | 48.558 | 20.219 | −5.640 | 1.00 | 17.63 | N |
| ATOM | 2040 | CA | GLU | A | 283 | 47.693 | 19.956 | −4.485 | 1.00 | 17.55 | C |
| ATOM | 2041 | CB | GLU | A | 283 | 47.744 | 18.489 | −4.076 | 1.00 | 17.90 | C |
| ATOM | 2042 | CG | GLU | A | 283 | 46.951 | 17.556 | −4.989 | 1.00 | 18.94 | C |
| ATOM | 2043 | CD | GLU | A | 283 | 47.298 | 16.099 | −4.752 | 1.00 | 21.25 | C |
| ATOM | 2044 | OE1 | GLU | A | 283 | 48.463 | 15.806 | −4.453 | 1.00 | 21.74 | O |
| ATOM | 2045 | OE2 | GLU | A | 283 | 46.399 | 15.240 | −4.852 | 1.00 | 26.34 | O |
| ATOM | 2046 | C | GLU | A | 283 | 47.994 | 20.850 | −3.277 | 1.00 | 16.99 | C |
| ATOM | 2047 | O | GLU | A | 283 | 47.090 | 21.208 | −2.519 | 1.00 | 16.78 | O |
| ATOM | 2048 | N | ILE | A | 284 | 49.260 | 21.208 | −3.109 | 1.00 | 16.34 | N |
| ATOM | 2049 | CA | ILE | A | 284 | 49.645 | 22.147 | −2.057 | 1.00 | 16.23 | C |
| ATOM | 2050 | CB | ILE | A | 284 | 51.182 | 22.296 | −1.977 | 1.00 | 15.68 | C |
| ATOM | 2051 | CG1 | ILE | A | 284 | 51.837 | 20.997 | −1.492 | 1.00 | 15.14 | C |
| ATOM | 2052 | CD1 | ILE | A | 284 | 53.373 | 20.968 | −1.576 | 1.00 | 13.98 | C |
| ATOM | 2053 | CG2 | ILE | A | 284 | 51.552 | 23.488 | −1.074 | 1.00 | 15.67 | C |
| ATOM | 2054 | C | ILE | A | 284 | 49.003 | 23.507 | −2.320 | 1.00 | 16.48 | C |
| ATOM | 2055 | O | ILE | A | 284 | 48.371 | 24.076 | −1.447 | 1.00 | 16.43 | O |
| ATOM | 2056 | N | GLN | A | 285 | 49.162 | 24.009 | −3.539 | 1.00 | 16.57 | N |
| ATOM | 2057 | CA | GLN | A | 285 | 48.677 | 25.335 | −3.872 | 1.00 | 17.32 | C |
| ATOM | 2058 | CB | GLN | A | 285 | 49.376 | 25.867 | −5.124 | 1.00 | 16.61 | C |
| ATOM | 2059 | CG | GLN | A | 285 | 50.848 | 26.173 | −4.858 | 1.00 | 16.82 | C |
| ATOM | 2060 | CD | GLN | A | 285 | 51.485 | 26.941 | −5.984 | 1.00 | 16.61 | C |
| ATOM | 2061 | OE1 | GLN | A | 285 | 51.643 | 26.408 | −7.087 | 1.00 | 16.49 | O |
| ATOM | 2062 | NE2 | GLN | A | 285 | 51.822 | 28.204 | −5.731 | 1.00 | 12.74 | N |
| ATOM | 2063 | C | GLN | A | 285 | 47.153 | 25.426 | −3.998 | 1.00 | 17.59 | C |
| ATOM | 2064 | O | GLN | A | 285 | 46.596 | 26.520 | −3.882 | 1.00 | 17.62 | O |
| ATOM | 2065 | N | ASN | A | 286 | 46.495 | 24.292 | −4.231 | 1.00 | 17.76 | N |
| ATOM | 2066 | CA | ASN | A | 286 | 45.038 | 24.227 | −4.189 | 1.00 | 18.67 | C |
| ATOM | 2067 | CB | ASN | A | 286 | 44.507 | 23.196 | −5.199 | 1.00 | 19.01 | C |
| ATOM | 2068 | CG | ASN | A | 286 | 44.599 | 23.683 | −6.644 | 1.00 | 20.31 | C |
| ATOM | 2069 | OD1 | ASN | A | 286 | 44.581 | 24.890 | −6.923 | 1.00 | 21.12 | O |
| ATOM | 2070 | ND2 | ASN | A | 286 | 44.697 | 22.746 | −7.566 | 1.00 | 21.21 | N |
| ATOM | 2071 | C | ASN | A | 286 | 44.473 | 23.948 | −2.787 | 1.00 | 19.15 | C |
| ATOM | 2072 | O | ASN | A | 286 | 43.253 | 23.959 | −2.585 | 1.00 | 18.66 | O |
| ATOM | 2073 | N | HIS | A | 287 | 45.362 | 23.711 | −1.820 | 1.00 | 19.57 | N |
| ATOM | 2074 | CA | HIS | A | 287 | 44.943 | 23.402 | −0.455 | 1.00 | 19.96 | C |
| ATOM | 2075 | CB | HIS | A | 287 | 46.161 | 23.008 | 0.398 | 1.00 | 20.42 | C |
| ATOM | 2076 | CG | HIS | A | 287 | 45.811 | 22.535 | 1.776 | 1.00 | 20.16 | C |
| ATOM | 2077 | ND1 | HIS | A | 287 | 45.771 | 21.201 | 2.120 | 1.00 | 21.24 | N |
| ATOM | 2078 | CE1 | HIS | A | 287 | 45.417 | 21.085 | 3.388 | 1.00 | 20.50 | C |
| ATOM | 2079 | NE2 | HIS | A | 287 | 45.224 | 22.298 | 3.878 | 1.00 | 21.14 | N |
| ATOM | 2080 | CD2 | HIS | A | 287 | 45.469 | 23.220 | 2.891 | 1.00 | 19.59 | C |
| ATOM | 2081 | C | HIS | A | 287 | 44.212 | 24.613 | 0.140 | 1.00 | 20.17 | C |
| ATOM | 2082 | O | HIS | A | 287 | 44.585 | 25.753 | −0.140 | 1.00 | 20.09 | O |
| ATOM | 2083 | N | PRO | A | 288 | 43.154 | 24.375 | 0.921 | 1.00 | 20.64 | N |
| ATOM | 2084 | CA | PRO | A | 288 | 42.399 | 25.468 | 1.540 | 1.00 | 20.94 | C |
| ATOM | 2085 | CB | PRO | A | 288 | 41.409 | 24.741 | 2.463 | 1.00 | 21.25 | C |
| ATOM | 2086 | CG | PRO | A | 288 | 41.229 | 23.429 | 1.836 | 1.00 | 21.49 | C |
| ATOM | 2087 | CD | PRO | A | 288 | 42.549 | 23.063 | 1.215 | 1.00 | 20.84 | C |
| ATOM | 2088 | C | PRO | A | 288 | 43.263 | 26.449 | 2.323 | 1.00 | 20.62 | C |
| ATOM | 2089 | O | PRO | A | 288 | 42.995 | 27.641 | 2.242 | 1.00 | 21.00 | O |
| ATOM | 2090 | N | TRP | A | 289 | 44.290 | 25.982 | 3.027 | 1.00 | 20.44 | N |
| ATOM | 2091 | CA | TRP | A | 289 | 45.144 | 26.903 | 3.785 | 1.00 | 20.73 | C |
| ATOM | 2092 | CB | TRP | A | 289 | 46.165 | 26.169 | 4.668 | 1.00 | 19.78 | C |
| ATOM | 2093 | CG | TRP | A | 289 | 46.932 | 27.139 | 5.535 | 1.00 | 18.93 | C |
| ATOM | 2094 | CD1 | TRP | A | 289 | 46.469 | 27.795 | 6.646 | 1.00 | 17.79 | C |
| ATOM | 2095 | NE1 | TRP | A | 289 | 47.450 | 28.617 | 7.152 | 1.00 | 17.69 | N |
| ATOM | 2096 | CE2 | TRP | A | 289 | 48.560 | 28.535 | 6.348 | 1.00 | 17.45 | C |
| ATOM | 2097 | CD2 | TRP | A | 289 | 48.265 | 27.614 | 5.316 | 1.00 | 16.35 | C |
| ATOM | 2098 | CE3 | TRP | A | 289 | 49.252 | 27.348 | 4.352 | 1.00 | 16.19 | C |
| ATOM | 2099 | CZ3 | TRP | A | 289 | 50.488 | 27.992 | 4.453 | 1.00 | 14.25 | C |
| ATOM | 2100 | CH2 | TRP | A | 289 | 50.753 | 28.894 | 5.498 | 1.00 | 15.51 | C |
| ATOM | 2101 | CZ2 | TRP | A | 289 | 49.805 | 29.181 | 6.453 | 1.00 | 16.21 | C |
| ATOM | 2102 | C | TRP | A | 289 | 45.868 | 27.924 | 2.897 | 1.00 | 21.43 | C |
| ATOM | 2103 | O | TRP | A | 289 | 46.219 | 29.005 | 3.371 | 1.00 | 21.35 | O |
| ATOM | 2104 | N | MET | A | 290 | 46.081 | 27.575 | 1.627 | 1.00 | 22.16 | N |
| ATOM | 2105 | CA | MET | A | 290 | 46.795 | 28.432 | 0.672 | 1.00 | 23.41 | C |
| ATOM | 2106 | CB | MET | A | 290 | 47.570 | 27.561 | −0.328 | 1.00 | 23.44 | C |
| ATOM | 2107 | CG | MET | A | 290 | 48.631 | 26.687 | 0.341 | 1.00 | 23.18 | C |
| ATOM | 2108 | SD | MET | A | 290 | 50.289 | 27.282 | −0.016 | 1.00 | 24.31 | S |
| ATOM | 2109 | CE | MET | A | 290 | 50.282 | 28.805 | 0.810 | 1.00 | 21.67 | C |
| ATOM | 2110 | C | MET | A | 290 | 45.935 | 29.456 | −0.093 | 1.00 | 24.66 | C |
| ATOM | 2111 | O | MET | A | 290 | 46.465 | 30.229 | −0.901 | 1.00 | 24.82 | O |
| ATOM | 2112 | N | GLN | A | 291 | 44.627 | 29.472 | 0.161 | 1.00 | 25.63 | N |
| ATOM | 2113 | CA | GLN | A | 291 | 43.725 | 30.390 | −0.541 | 1.00 | 27.47 | C |
| ATOM | 2114 | CB | GLN | A | 291 | 42.263 | 29.932 | −0.399 | 1.00 | 28.12 | C |
| ATOM | 2115 | CG | GLN | A | 291 | 41.931 | 28.612 | −1.133 | 1.00 | 30.95 | C |
| ATOM | 2116 | CD | GLN | A | 291 | 42.797 | 28.376 | −2.378 | 1.00 | 35.68 | C |
| ATOM | 2117 | OE1 | GLN | A | 291 | 42.599 | 29.038 | −3.414 | 1.00 | 37.06 | O |
| ATOM | 2118 | NE2 | GLN | A | 291 | 43.766 | 27.441 | −2.277 | 1.00 | 34.40 | N |
| ATOM | 2119 | C | GLN | A | 291 | 43.879 | 31.844 | −0.094 | 1.00 | 27.73 | C |
| ATOM | 2120 | O | GLN | A | 291 | 44.222 | 32.122 | 1.059 | 1.00 | 28.09 | O |
| ATOM | 2121 | N | ASP | A | 292 | 43.651 | 32.765 | −1.026 | 1.00 | 28.22 | N |
| ATOM | 2122 | CA | ASP | A | 292 | 43.678 | 34.207 | −0.750 | 1.00 | 28.34 | C |
| ATOM | 2123 | CB | ASP | A | 292 | 42.553 | 34.596 | 0.224 | 1.00 | 29.02 | C |
| ATOM | 2124 | CG | ASP | A | 292 | 41.176 | 34.236 | −0.308 | 1.00 | 31.45 | C |
| ATOM | 2125 | OD1 | ASP | A | 292 | 40.817 | 34.731 | −1.402 | 1.00 | 33.30 | O |
| ATOM | 2126 | OD2 | ASP | A | 292 | 40.400 | 33.452 | 0.291 | 1.00 | 34.49 | O |
| ATOM | 2127 | C | ASP | A | 292 | 45.027 | 34.718 | −0.245 | 1.00 | 27.65 | C |
| ATOM | 2128 | O | ASP | A | 292 | 45.098 | 35.446 | 0.761 | 1.00 | 27.28 | O |
| ATOM | 2129 | N | VAL | A | 293 | 46.094 | 34.334 | −0.946 | 1.00 | 26.88 | N |
| ATOM | 2130 | CA | VAL | A | 293 | 47.429 | 34.808 | −0.622 | 1.00 | 25.72 | C |
| ATOM | 2131 | CB | VAL | A | 293 | 48.538 | 34.025 | −1.396 | 1.00 | 26.36 | C |
| ATOM | 2132 | CG1 | VAL | A | 293 | 48.546 | 34.374 | −2.881 | 1.00 | 26.33 | C |
| ATOM | 2133 | CG2 | VAL | A | 293 | 49.912 | 34.299 | −0.799 | 1.00 | 24.82 | C |
| ATOM | 2134 | C | VAL | A | 293 | 47.550 | 36.311 | −0.877 | 1.00 | 25.50 | C |
| ATOM | 2135 | O | VAL | A | 293 | 47.007 | 36.824 | −1.848 | 1.00 | 24.70 | O |
| ATOM | 2136 | N | LEU | A | 294 | 48.261 | 37.004 | 0.009 | 1.00 | 25.07 | N |
| ATOM | 2137 | CA | LEU | A | 294 | 48.630 | 38.392 | −0.226 | 1.00 | 25.15 | C |
| ATOM | 2138 | CB | LEU | A | 294 | 49.280 | 38.998 | 1.017 | 1.00 | 24.83 | C |
| ATOM | 2139 | CG | LEU | A | 294 | 48.500 | 39.140 | 2.329 | 1.00 | 24.86 | C |
| ATOM | 2140 | CD1 | LEU | A | 294 | 49.412 | 39.783 | 3.371 | 1.00 | 22.85 | C |
| ATOM | 2141 | CD2 | LEU | A | 294 | 47.199 | 39.941 | 2.148 | 1.00 | 24.42 | C |
| ATOM | 2142 | C | LEU | A | 294 | 49.621 | 38.487 | −1.384 | 1.00 | 25.66 | C |
| ATOM | 2143 | O | LEU | A | 294 | 50.437 | 37.585 | −1.598 | 1.00 | 25.15 | O |
| ATOM | 2144 | N | LEU | A | 295 | 49.539 | 39.585 | −2.128 | 1.00 | 26.06 | N |
| ATOM | 2145 | CA | LEU | A | 295 | 50.587 | 39.950 | −3.071 | 1.00 | 26.73 | C |
| ATOM | 2146 | CB | LEU | A | 295 | 50.122 | 41.106 | −3.981 | 1.00 | 27.23 | C |
| ATOM | 2147 | CG | LEU | A | 295 | 48.775 | 40.944 | −4.717 | 1.00 | 29.21 | C |
| ATOM | 2148 | CD1 | LEU | A | 295 | 48.330 | 42.237 | −5.412 | 1.00 | 31.53 | C |
| ATOM | 2149 | CD2 | LEU | A | 295 | 48.829 | 39.801 | −5.721 | 1.00 | 31.44 | C |
| ATOM | 2150 | C | LEU | A | 295 | 51.841 | 40.337 | −2.265 | 1.00 | 26.44 | C |
| ATOM | 2151 | O | LEU | A | 295 | 51.729 | 40.733 | −1.103 | 1.00 | 25.11 | O |
| ATOM | 2152 | N | PRO | A | 296 | 53.028 | 40.170 | −2.851 | 1.00 | 27.04 | N |
| ATOM | 2153 | CA | PRO | A | 296 | 54.277 | 40.535 | −2.164 | 1.00 | 27.59 | C |
| ATOM | 2154 | CB | PRO | A | 296 | 55.331 | 40.358 | −3.250 | 1.00 | 27.65 | C |
| ATOM | 2155 | CG | PRO | A | 296 | 54.772 | 39.247 | −4.091 | 1.00 | 27.68 | C |
| ATOM | 2156 | CD | PRO | A | 296 | 53.292 | 39.564 | −4.171 | 1.00 | 26.80 | C |
| ATOM | 2157 | C | PRO | A | 296 | 54.265 | 41.964 | −1.623 | 1.00 | 28.49 | C |
| ATOM | 2158 | O | PRO | A | 296 | 54.608 | 42.151 | −0.459 | 1.00 | 28.36 | O |
| ATOM | 2159 | N | GLN | A | 297 | 53.854 | 42.942 | −2.430 | 1.00 | 29.34 | N |
| ATOM | 2160 | CA | GLN | A | 297 | 53.801 | 44.328 | −1.960 | 1.00 | 30.65 | C |
| ATOM | 2161 | CB | GLN | A | 297 | 53.467 | 45.305 | −3.102 | 1.00 | 31.10 | C |
| ATOM | 2162 | CG | GLN | A | 297 | 53.783 | 46.766 | −2.787 | 1.00 | 33.83 | C |
| ATOM | 2163 | CD | GLN | A | 297 | 55.239 | 46.993 | −2.374 | 1.00 | 37.47 | C |
| ATOM | 2164 | OE1 | GLN | A | 297 | 56.158 | 46.742 | −3.153 | 1.00 | 38.85 | O |
| ATOM | 2165 | NE2 | GLN | A | 297 | 55.444 | 47.468 | −1.147 | 1.00 | 39.03 | N |
| ATOM | 2166 | C | GLN | A | 297 | 52.830 | 44.490 | −0.782 | 1.00 | 30.41 | C |
| ATOM | 2167 | O | GLN | A | 297 | 53.174 | 45.125 | 0.210 | 1.00 | 30.58 | O |
| ATOM | 2168 | N | GLU | A | 298 | 51.640 | 43.900 | −0.891 | 1.00 | 30.38 | N |
| ATOM | 2169 | CA | GLU | A | 298 | 50.699 | 43.839 | 0.235 | 1.00 | 30.73 | C |
| ATOM | 2170 | CB | GLU | A | 298 | 49.479 | 42.981 | −0.103 | 1.00 | 31.10 | C |
| ATOM | 2171 | CG | GLU | A | 298 | 48.534 | 43.558 | −1.141 | 1.00 | 33.59 | C |
| ATOM | 2172 | CD | GLU | A | 298 | 47.270 | 42.722 | −1.289 | 1.00 | 37.83 | C |
| ATOM | 2173 | OE1 | GLU | A | 298 | 47.369 | 41.480 | −1.435 | 1.00 | 36.99 | O |
| ATOM | 2174 | OE2 | GLU | A | 298 | 46.166 | 43.313 | −1.272 | 1.00 | 40.82 | O |
| ATOM | 2175 | C | GLU | A | 298 | 51.378 | 43.269 | 1.485 | 1.00 | 30.06 | C |
| ATOM | 2176 | O | GLU | A | 298 | 51.258 | 43.837 | 2.577 | 1.00 | 30.09 | O |
| ATOM | 2177 | N | THR | A | 299 | 52.096 | 42.156 | 1.301 | 1.00 | 28.83 | N |
| ATOM | 2178 | CA | THR | A | 299 | 52.857 | 41.496 | 2.360 | 1.00 | 27.61 | C |
| ATOM | 2179 | CB | THR | A | 299 | 53.619 | 40.265 | 1.782 | 1.00 | 27.45 | C |
| ATOM | 2180 | OG1 | THR | A | 299 | 52.690 | 39.346 | 1.192 | 1.00 | 25.03 | O |
| ATOM | 2181 | CG2 | THR | A | 299 | 54.269 | 39.447 | 2.897 | 1.00 | 26.87 | C |
| ATOM | 2182 | C | THR | A | 299 | 53.840 | 42.442 | 3.062 | 1.00 | 27.70 | C |
| ATOM | 2183 | O | THR | A | 299 | 53.890 | 42.487 | 4.289 | 1.00 | 27.42 | O |
| ATOM | 2184 | N | ALA | A | 300 | 54.626 | 43.177 | 2.278 | 1.00 | 27.78 | N |
| ATOM | 2185 | CA | ALA | A | 300 | 55.622 | 44.093 | 2.819 | 1.00 | 28.26 | C |
| ATOM | 2186 | CB | ALA | A | 300 | 56.517 | 44.627 | 1.706 | 1.00 | 28.09 | C |
| ATOM | 2187 | C | ALA | A | 300 | 54.972 | 45.250 | 3.588 | 1.00 | 28.73 | C |
| ATOM | 2188 | O | ALA | A | 300 | 55.444 | 45.636 | 4.658 | 1.00 | 28.08 | O |
| ATOM | 2189 | N | GLU | A | 301 | 53.884 | 45.785 | 3.040 | 1.00 | 29.58 | N |
| ATOM | 2190 | CA | GLU | A | 301 | 53.169 | 46.894 | 3.668 | 1.00 | 31.18 | C |
| ATOM | 2191 | CB | GLU | A | 301 | 52.063 | 47.433 | 2.738 | 1.00 | 31.45 | C |
| ATOM | 2192 | CG | GLU | A | 301 | 52.592 | 48.250 | 1.555 | 1.00 | 34.28 | C |
| ATOM | 2193 | CD | GLU | A | 301 | 51.553 | 48.517 | 0.460 | 1.00 | 37.15 | C |
| ATOM | 2194 | OE1 | GLU | A | 301 | 50.659 | 47.668 | 0.219 | 1.00 | 37.47 | O |
| ATOM | 2195 | OE2 | GLU | A | 301 | 51.642 | 49.589 | −0.177 | 1.00 | 38.55 | O |
| ATOM | 2196 | C | GLU | A | 301 | 52.610 | 46.488 | 5.042 | 1.00 | 31.10 | C |
| ATOM | 2197 | O | GLU | A | 301 | 52.779 | 47.211 | 6.019 | 1.00 | 31.18 | O |
| ATOM | 2198 | N | ILE | A | 302 | 51.989 | 45.311 | 5.107 | 1.00 | 31.28 | N |
| ATOM | 2199 | CA | ILE | A | 302 | 51.371 | 44.823 | 6.340 | 1.00 | 31.59 | C |
| ATOM | 2200 | CB | ILE | A | 302 | 50.284 | 43.767 | 6.019 | 1.00 | 31.30 | C |
| ATOM | 2201 | CG1 | ILE | A | 302 | 49.201 | 44.378 | 5.115 | 1.00 | 31.02 | C |
| ATOM | 2202 | CD1 | ILE | A | 302 | 48.293 | 43.357 | 4.435 | 1.00 | 29.87 | C |
| ATOM | 2203 | CG2 | ILE | A | 302 | 49.673 | 43.195 | 7.311 | 1.00 | 30.80 | C |
| ATOM | 2204 | C | ILE | A | 302 | 52.384 | 44.286 | 7.373 | 1.00 | 32.02 | C |
| ATOM | 2205 | O | ILE | A | 302 | 52.263 | 44.577 | 8.560 | 1.00 | 31.80 | O |
| ATOM | 2206 | N | HIS | A | 303 | 53.391 | 43.544 | 6.909 | 1.00 | 32.60 | N |
| ATOM | 2207 | CA | HIS | A | 303 | 54.253 | 42.750 | 7.790 | 1.00 | 33.26 | C |
| ATOM | 2208 | CB | HIS | A | 303 | 54.176 | 41.282 | 7.379 | 1.00 | 32.20 | C |
| ATOM | 2209 | CG | HIS | A | 303 | 52.832 | 40.662 | 7.606 | 1.00 | 29.68 | C |
| ATOM | 2210 | ND1 | HIS | A | 303 | 52.385 | 40.293 | 8.857 | 1.00 | 27.26 | N |
| ATOM | 2211 | CE1 | HIS | A | 303 | 51.171 | 39.780 | 8.755 | 1.00 | 27.29 | C |
| ATOM | 2212 | NE2 | HIS | A | 303 | 50.819 | 39.796 | 7.481 | 1.00 | 26.60 | N |
| ATOM | 2213 | CD2 | HIS | A | 303 | 51.839 | 40.346 | 6.743 | 1.00 | 26.43 | C |
| ATOM | 2214 | C | HIS | A | 303 | 55.723 | 43.174 | 7.849 | 1.00 | 35.09 | C |
| ATOM | 2215 | O | HIS | A | 303 | 56.435 | 42.838 | 8.796 | 1.00 | 34.73 | O |
| ATOM | 2216 | N | LEU | A | 304 | 56.181 | 43.889 | 6.827 | 1.00 | 37.42 | N |
| ATOM | 2217 | CA | LEU | A | 304 | 57.588 | 44.256 | 6.724 | 1.00 | 40.17 | C |
| ATOM | 2218 | CB | LEU | A | 304 | 58.187 | 43.707 | 5.421 | 1.00 | 39.43 | C |
| ATOM | 2219 | CG | LEU | A | 304 | 58.574 | 42.224 | 5.234 | 1.00 | 38.74 | C |
| ATOM | 2220 | CD1 | LEU | A | 304 | 57.830 | 41.238 | 6.125 | 1.00 | 34.54 | C |
| ATOM | 2221 | CD2 | LEU | A | 304 | 58.465 | 41.807 | 3.760 | 1.00 | 35.74 | C |
| ATOM | 2222 | C | LEU | A | 304 | 57.751 | 45.774 | 6.796 | 1.00 | 42.83 | C |
| ATOM | 2223 | O | LEU | A | 304 | 58.861 | 46.286 | 6.661 | 1.00 | 43.07 | O |
| ATOM | 2224 | N | HIS | A | 305 | 56.629 | 46.462 | 7.033 | 1.00 | 46.30 | N |
| ATOM | 2225 | CA | HIS | A | 305 | 56.516 | 47.933 | 7.107 | 1.00 | 49.71 | C |
| ATOM | 2226 | CB | HIS | A | 305 | 56.747 | 48.459 | 8.545 | 1.00 | 50.37 | C |
| ATOM | 2227 | CG | HIS | A | 305 | 58.085 | 48.106 | 9.125 | 1.00 | 53.30 | C |
| ATOM | 2228 | ND1 | HIS | A | 305 | 58.344 | 46.886 | 9.716 | 1.00 | 56.26 | N |
| ATOM | 2229 | CE1 | HIS | A | 305 | 59.597 | 46.860 | 10.138 | 1.00 | 57.48 | C |
| ATOM | 2230 | NE2 | HIS | A | 305 | 60.159 | 48.022 | 9.847 | 1.00 | 57.58 | N |
| ATOM | 2231 | CD2 | HIS | A | 305 | 59.234 | 48.820 | 9.216 | 1.00 | 56.22 | C |
| ATOM | 2232 | C | HIS | A | 305 | 57.339 | 48.707 | 6.063 | 1.00 | 50.97 | C |
| ATOM | 2233 | O | HIS | A | 305 | 58.423 | 49.221 | 6.359 | 1.00 | 51.59 | O |
| ATOM | 2234 | N | SER | A | 306 | 56.795 | 48.799 | 4.850 | 1.00 | 52.46 | N |
| ATOM | 2235 | CA | SER | A | 306 | 57.518 | 49.345 | 3.693 | 1.00 | 53.65 | C |
| ATOM | 2236 | CB | SER | A | 306 | 56.768 | 49.009 | 2.401 | 1.00 | 53.75 | C |
| ATOM | 2237 | OG | SER | A | 306 | 57.340 | 47.873 | 1.784 | 1.00 | 54.33 | O |
| ATOM | 2238 | C | SER | A | 306 | 57.820 | 50.851 | 3.763 | 1.00 | 54.12 | C |
| ATOM | 2239 | O | SER | A | 306 | 58.964 | 51.291 | 3.600 | 1.00 | 54.51 | O |
| ATOM | 2240 | OXT | SER | A | 306 | 56.943 | 51.696 | 3.974 | 1.00 | 54.48 | O |
| ATOM | 2241 | O1A | ANP | L | 1 | 74.739 | 30.562 | −0.833 | 1.00 | 18.02 | O |
| ATOM | 2242 | PA | ANP | L | 1 | 74.774 | 30.444 | 0.630 | 1.00 | 19.01 | P |
| ATOM | 2243 | O2A | ANP | L | 1 | 73.576 | 29.828 | 1.256 | 1.00 | 17.67 | O |
| ATOM | 2244 | O3A | ANP | L | 1 | 76.090 | 29.652 | 0.938 | 1.00 | 19.50 | O |
| ATOM | 2245 | PB | ANP | L | 1 | 76.391 | 28.426 | 1.887 | 1.00 | 21.38 | P |
| ATOM | 2246 | O1B | ANP | L | 1 | 77.321 | 27.642 | 1.069 | 1.00 | 18.82 | O |
| ATOM | 2247 | O2B | ANP | L | 1 | 77.348 | 29.007 | 2.991 | 1.00 | 24.75 | O |
| ATOM | 2248 | N3B | ANP | L | 1 | 75.190 | 27.617 | 2.664 | 1.00 | 20.02 | N |
| ATOM | 2249 | PG | ANP | L | 1 | 73.526 | 27.764 | 2.959 | 1.00 | 31.62 | P |
| ATOM | 2250 | O3G | ANP | L | 1 | 73.022 | 29.016 | 3.938 | 1.00 | 19.18 | O |
| ATOM | 2251 | O2G | ANP | L | 1 | 73.054 | 27.935 | 1.600 | 1.00 | 20.72 | O |
| ATOM | 2252 | O1G | ANP | L | 1 | 72.907 | 26.389 | 3.404 | 1.00 | 20.30 | O |
| ATOM | 2253 | O5* | ANP | L | 1 | 74.945 | 31.880 | 1.324 | 1.00 | 18.06 | O |
| ATOM | 2254 | C5* | ANP | L | 1 | 75.248 | 31.923 | 2.714 | 1.00 | 17.09 | C |
| ATOM | 2255 | C4* | ANP | L | 1 | 74.482 | 33.091 | 3.324 | 1.00 | 18.01 | C |
| ATOM | 2256 | O4* | ANP | L | 1 | 74.771 | 34.292 | 2.621 | 1.00 | 19.09 | O |
| ATOM | 2257 | C1* | ANP | L | 1 | 73.660 | 35.124 | 2.442 | 1.00 | 17.31 | C |
| ATOM | 2258 | C2* | ANP | L | 1 | 72.535 | 34.397 | 3.160 | 1.00 | 18.01 | C |
| ATOM | 2259 | O2* | ANP | L | 1 | 72.451 | 34.900 | 4.487 | 1.00 | 19.01 | O |
| ATOM | 2260 | C3* | ANP | L | 1 | 72.983 | 32.937 | 3.178 | 1.00 | 17.74 | C |
| ATOM | 2261 | O3* | ANP | L | 1 | 72.429 | 32.083 | 4.163 | 1.00 | 17.16 | O |
| ATOM | 2262 | N9 | ANP | L | 1 | 73.486 | 35.319 | 0.979 | 1.00 | 17.49 | N |
| ATOM | 2263 | C8 | ANP | L | 1 | 73.739 | 34.403 | −0.019 | 1.00 | 15.85 | C |
| ATOM | 2264 | N7 | ANP | L | 1 | 73.458 | 34.943 | −1.228 | 1.00 | 14.30 | N |
| ATOM | 2265 | C5 | ANP | L | 1 | 73.025 | 36.193 | −1.045 | 1.00 | 15.53 | C |
| ATOM | 2266 | C6 | ANP | L | 1 | 72.607 | 37.177 | −1.929 | 1.00 | 16.13 | C |
| ATOM | 2267 | N6 | ANP | L | 1 | 72.542 | 36.951 | −3.254 | 1.00 | 14.38 | N |
| ATOM | 2268 | C4 | ANP | L | 1 | 73.039 | 36.448 | 0.334 | 1.00 | 15.81 | C |
| ATOM | 2269 | N3 | ANP | L | 1 | 72.632 | 37.646 | 0.795 | 1.00 | 17.04 | N |
| ATOM | 2270 | C2 | ANP | L | 1 | 72.219 | 38.650 | −0.068 | 1.00 | 17.00 | C |
| ATOM | 2271 | N1 | ANP | L | 1 | 72.213 | 38.406 | −1.417 | 1.00 | 16.49 | N |
| ATOM | 2272 | O | HOH | W | 1 | 63.572 | 15.756 | 8.058 | 1.00 | 26.33 | O |
| ATOM | 2273 | O | HOH | W | 2 | 61.017 | 10.214 | −2.362 | 1.00 | 24.27 | O |
| ATOM | 2274 | O | HOH | W | 3 | 54.457 | 23.038 | −11.444 | 1.00 | 30.92 | O |
| ATOM | 2275 | O | HOH | W | 4 | 63.756 | 21.549 | −5.585 | 1.00 | 27.71 | O |
| ATOM | 2276 | O | HOH | W | 5 | 63.196 | 11.516 | −9.068 | 1.00 | 26.46 | O |
| ATOM | 2277 | O | HOH | W | 6 | 58.424 | 12.040 | −6.552 | 1.00 | 34.61 | O |
| ATOM | 2278 | O | HOH | W | 7 | 54.593 | 37.425 | 12.022 | 1.00 | 32.21 | O |
| ATOM | 2279 | O | HOH | W | 8 | 71.368 | 23.298 | −3.142 | 1.00 | 29.05 | O |
| ATOM | 2280 | O | HOH | W | 9 | 64.911 | 20.478 | −0.663 | 1.00 | 26.42 | O |
| ATOM | 2281 | O | HOH | W | 10 | 43.132 | 26.500 | 18.254 | 1.00 | 34.21 | O |
| ATOM | 2282 | O | HOH | W | 11 | 64.667 | 17.153 | −3.465 | 1.00 | 26.68 | O |
| ATOM | 2283 | O | HOH | W | 12 | 75.478 | 24.941 | 1.494 | 1.00 | 29.16 | O |
| ATOM | 2284 | O | HOH | W | 13 | 63.267 | 18.804 | 11.098 | 1.00 | 24.60 | O |
| ATOM | 2285 | O | HOH | W | 14 | 47.333 | 35.497 | 10.172 | 1.00 | 39.07 | O |
| ATOM | 2286 | O | HOH | W | 15 | 41.592 | 25.798 | 13.188 | 1.00 | 29.60 | O |
| ATOM | 2287 | O | HOH | W | 16 | 46.216 | 35.678 | 3.186 | 1.00 | 30.38 | O |
| ATOM | 2288 | O | HOH | W | 17 | 73.656 | 23.760 | −0.205 | 1.00 | 33.83 | O |
| ATOM | 2289 | O | HOH | W | 18 | 54.975 | 14.884 | −3.637 | 1.00 | 27.43 | O |
| ATOM | 2290 | O | HOH | W | 19 | 58.350 | 33.360 | −6.094 | 1.00 | 29.01 | O |
| ATOM | 2291 | O | HOH | W | 20 | 58.458 | 11.832 | 15.075 | 1.00 | 34.06 | O |
| ATOM | 2292 | O | HOH | W | 21 | 48.299 | 24.286 | 17.311 | 1.00 | 29.81 | O |
| ATOM | 2293 | O | HOH | W | 22 | 67.356 | 21.562 | 3.234 | 1.00 | 31.16 | O |
| ATOM | 2294 | O | HOH | W | 23 | 84.186 | 28.990 | 2.689 | 1.00 | 31.81 | O |
| ATOM | 2295 | O | HOH | W | 24 | 43.050 | 31.228 | 3.507 | 1.00 | 47.48 | O |
| ATOM | 2296 | O | HOH | W | 25 | 88.316 | 32.615 | −4.360 | 1.00 | 32.51 | O |
| ATOM | 2297 | O | HOH | W | 26 | 71.447 | 43.185 | −7.672 | 1.00 | 43.19 | O |
| ATOM | 2298 | O | HOH | W | 27 | 64.646 | 19.993 | −3.620 | 1.00 | 28.98 | O |
| ATOM | 2299 | O | HOH | W | 28 | 71.618 | 43.781 | 0.688 | 1.00 | 40.30 | O |
| ATOM | 2300 | O | HOH | W | 29 | 70.325 | 37.710 | 6.677 | 1.00 | 31.97 | O |
| ATOM | 2301 | O | HOH | W | 30 | 71.184 | 18.590 | 9.525 | 1.00 | 35.40 | O |
| ATOM | 2302 | O | HOH | W | 31 | 53.890 | 12.402 | −3.734 | 1.00 | 32.44 | O |
| ATOM | 2303 | O | HOH | W | 32 | 52.246 | 19.524 | −9.419 | 1.00 | 35.84 | O |
| ATOM | 2304 | O | HOH | W | 33 | 40.639 | 26.398 | 16.837 | 1.00 | 35.65 | O |
| ATOM | 2305 | O | HOH | W | 34 | 60.620 | 13.344 | −8.811 | 1.00 | 44.92 | O |
| ATOM | 2306 | O | HOH | W | 35 | 75.110 | 44.117 | 2.424 | 1.00 | 40.04 | O |
| ATOM | 2307 | O | HOH | W | 36 | 74.471 | 37.990 | 7.461 | 1.00 | 40.83 | O |
| ATOM | 2308 | O | HOH | W | 37 | 59.228 | 35.799 | −5.068 | 1.00 | 33.92 | O |
| ATOM | 2309 | O | HOH | W | 38 | 57.123 | 17.309 | 16.065 | 1.00 | 40.82 | O |
| ATOM | 2310 | O | HOH | W | 39 | 73.994 | 32.523 | −2.675 | 1.00 | 33.30 | O |
| ATOM | 2311 | O | HOH | W | 40 | 69.993 | 44.693 | 3.957 | 1.00 | 50.42 | O |
| ATOM | 2312 | O | HOH | W | 41 | 65.864 | 18.120 | 0.377 | 1.00 | 37.77 | O |
| ATOM | 2313 | O | HOH | W | 42 | 48.834 | 35.741 | 2.440 | 1.00 | 31.77 | O |
| ATOM | 2314 | O | HOH | W | 43 | 52.185 | 7.956 | 4.576 | 1.00 | 38.71 | O |
| ATOM | 2315 | O | HOH | W | 44 | 64.765 | 11.133 | −15.777 | 1.00 | 33.65 | O |
| ATOM | 2316 | O | HOH | W | 45 | 48.197 | 17.129 | −9.023 | 1.00 | 35.25 | O |
| ATOM | 2317 | O | HOH | W | 46 | 71.559 | 9.704 | −7.782 | 1.00 | 42.63 | O |
| ATOM | 2318 | O | HOH | W | 47 | 72.838 | 31.092 | −4.833 | 1.00 | 32.07 | O |
| ATOM | 2319 | O | HOH | W | 48 | 54.340 | 33.741 | −9.311 | 1.00 | 38.01 | O |
| ATOM | 2320 | O | HOH | W | 49 | 54.223 | 11.905 | 9.111 | 1.00 | 33.22 | O |
| ATOM | 2321 | O | HOH | W | 50 | 52.887 | 36.641 | −1.776 | 1.00 | 38.05 | O |
| ATOM | 2322 | O | HOH | W | 51 | 58.033 | 32.102 | 18.276 | 1.00 | 41.20 | O |
| ATOM | 2323 | O | HOH | W | 52 | 58.764 | 11.092 | 17.987 | 1.00 | 35.48 | O |
| ATOM | 2324 | O | HOH | W | 53 | 56.210 | 29.247 | −10.737 | 1.00 | 40.52 | O |
| ATOM | 2325 | O | HOH | W | 54 | 75.583 | 29.566 | 5.684 | 1.00 | 42.12 | O |
| ATOM | 2326 | O | HOH | W | 55 | 82.299 | 27.704 | 4.152 | 1.00 | 43.41 | O |
| ATOM | 2327 | O | HOH | W | 56 | 61.670 | 6.087 | 15.210 | 1.00 | 42.46 | O |
| ATOM | 2328 | O | HOH | W | 57 | 41.909 | 26.005 | 9.492 | 1.00 | 38.52 | O |
| ATOM | 2329 | O | HOH | W | 58 | 72.941 | 15.417 | 4.788 | 1.00 | 53.05 | O |
| ATOM | 2330 | O | HOH | W | 59 | 56.478 | 27.573 | −12.787 | 1.00 | 37.09 | O |
| ATOM | 2331 | O | HOH | W | 60 | 83.158 | 40.743 | 6.932 | 1.00 | 41.95 | O |
| ATOM | 2332 | O | HOH | W | 61 | 44.574 | 20.141 | −2.416 | 1.00 | 38.16 | O |
| ATOM | 2333 | O | HOH | W | 62 | 51.818 | 13.854 | 13.409 | 1.00 | 36.87 | O |
| ATOM | 2334 | O | HOH | W | 63 | 56.901 | 22.491 | −11.879 | 1.00 | 46.71 | O |
| ATOM | 2335 | O | HOH | W | 64 | 46.066 | 31.890 | −3.335 | 1.00 | 46.54 | O |
| ATOM | 2336 | O | HOH | W | 65 | 46.390 | 17.471 | 11.120 | 1.00 | 41.50 | O |
| ATOM | 2337 | O | HOH | W | 66 | 73.021 | 18.047 | 7.679 | 1.00 | 45.56 | O |
| ATOM | 2338 | O | HOH | W | 67 | 56.272 | 6.117 | −3.207 | 1.00 | 47.16 | O |
| ATOM | 2339 | O | HOH | W | 68 | 78.807 | 46.222 | 0.194 | 1.00 | 43.86 | O |
| ATOM | 2340 | O | HOH | W | 69 | 70.343 | 14.512 | −11.989 | 1.00 | 41.06 | O |
| ATOM | 2341 | O | HOH | W | 70 | 43.908 | 38.440 | 0.670 | 1.00 | 53.02 | O |
| ATOM | 2342 | O | HOH | W | 71 | 40.352 | 28.430 | 2.051 | 1.00 | 45.97 | O |
| ATOM | 2343 | O | HOH | W | 72 | 44.496 | 19.095 | 11.235 | 1.00 | 54.47 | O |
| ATOM | 2344 | O | HOH | W | 73 | 47.165 | 15.788 | 6.720 | 1.00 | 41.01 | O |
| ATOM | 2345 | O | HOH | W | 74 | 56.445 | 43.287 | −5.157 | 1.00 | 44.15 | O |
| ATOM | 2346 | O | HOH | W | 75 | 73.363 | 25.539 | −17.736 | 1.00 | 50.03 | O |
| ATOM | 2347 | O | HOH | W | 76 | 67.665 | 14.838 | −15.990 | 1.00 | 47.37 | O |
| ATOM | 2348 | O | HOH | W | 77 | 77.512 | 31.796 | 8.477 | 1.00 | 43.71 | O |
| ATOM | 2349 | O | HOH | W | 78 | 64.562 | 45.570 | −0.458 | 1.00 | 42.82 | O |
| ATOM | 2350 | O | HOH | W | 79 | 72.601 | 12.906 | 5.087 | 1.00 | 41.65 | O |
| ATOM | 2351 | O | HOH | W | 80 | 64.569 | 29.017 | 13.834 | 1.00 | 46.57 | O |
| ATOM | 2352 | O | HOH | W | 81 | 58.851 | 5.715 | −3.038 | 1.00 | 36.10 | O |
| ATOM | 2353 | O | HOH | W | 82 | 66.378 | 16.370 | −1.785 | 1.00 | 36.18 | O |
| ATOM | 2354 | O | HOH | W | 83 | 52.161 | 13.315 | 8.870 | 1.00 | 38.72 | O |
| ATOM | 2355 | O | HOH | W | 84 | 84.302 | 27.201 | −0.028 | 1.00 | 44.09 | O |
| ATOM | 2356 | O | HOH | W | 85 | 49.501 | 13.263 | −4.243 | 1.00 | 40.50 | O |
| ATOM | 2357 | O | HOH | W | 86 | 63.118 | 41.154 | −5.640 | 1.00 | 44.61 | O |
| ATOM | 2358 | O | HOH | W | 87 | 75.334 | 10.847 | −0.667 | 1.00 | 41.03 | O |
| ATOM | 2359 | O | HOH | W | 88 | 51.946 | 9.440 | 7.089 | 1.00 | 44.13 | O |
| ATOM | 2360 | O | HOH | W | 89 | 46.051 | 15.476 | 9.731 | 1.00 | 44.74 | O |
| ATOM | 2361 | O | HOH | W | 90 | 60.662 | 7.651 | −3.345 | 1.00 | 33.21 | O |
| ATOM | 2362 | O | HOH | W | 91 | 78.926 | 37.602 | 8.589 | 1.00 | 45.81 | O |
| ATOM | 2363 | O | HOH | W | 92 | 83.687 | 38.788 | 8.645 | 1.00 | 42.46 | O |
| ATOM | 2364 | O | HOH | W | 93 | 65.774 | 37.305 | −9.200 | 1.00 | 42.61 | O |
| ATOM | 2365 | O | HOH | W | 94 | 48.890 | 32.798 | 13.190 | 1.00 | 44.81 | O |
| ATOM | 2366 | O | HOH | W | 95 | 71.057 | 6.982 | 7.124 | 1.00 | 46.01 | O |
| ATOM | 2367 | O | HOH | W | 96 | 73.156 | 42.259 | 2.367 | 1.00 | 41.64 | O |
| ATOM | 2368 | O | HOH | W | 97 | 56.031 | 35.393 | 16.920 | 1.00 | 47.09 | O |
| ATOM | 2369 | O | HOH | W | 98 | 90.130 | 23.863 | −3.527 | 1.00 | 56.29 | O |
| ATOM | 2370 | O | HOH | W | 99 | 64.199 | 16.375 | 1.499 | 1.00 | 32.31 | O |
| ATOM | 2371 | O | HOH | W | 100 | 52.185 | 30.882 | 13.804 | 1.00 | 45.03 | O |
| ATOM | 2372 | O | HOH | W | 101 | 78.245 | 25.957 | −3.060 | 1.00 | 37.82 | O |
| ATOM | 2373 | O | HOH | W | 102 | 70.395 | 32.498 | −12.472 | 1.00 | 40.91 | O |
| ATOM | 2374 | O | HOH | W | 103 | 76.497 | 26.635 | −1.230 | 1.00 | 45.90 | O |
| ATOM | 2375 | O | HOH | W | 104 | 53.869 | 39.933 | 10.992 | 1.00 | 48.40 | O |
| ATOM | 2376 | O | HOH | W | 105 | 52.957 | 42.953 | −5.317 | 1.00 | 43.64 | O |
| ATOM | 2377 | O | HOH | W | 106 | 81.062 | 46.768 | −1.405 | 1.00 | 51.11 | O |
| ATOM | 2378 | O | HOH | W | 107 | 85.023 | 38.607 | −2.261 | 1.00 | 44.62 | O |
| ATOM | 2379 | O | HOH | W | 108 | 55.351 | 15.949 | −6.982 | 1.00 | 56.30 | O |
| ATOM | 2380 | O | HOH | W | 109 | 72.893 | 16.276 | −11.510 | 1.00 | 40.24 | O |
| ATOM | 2381 | O | HOH | W | 110 | 64.150 | 29.039 | 11.361 | 1.00 | 44.87 | O |
| ATOM | 2382 | O | HOH | W | 111 | 70.497 | 11.613 | 14.584 | 1.00 | 42.70 | O |
| ATOM | 2383 | O | HOH | W | 112 | 47.743 | 30.590 | 14.200 | 1.00 | 41.84 | O |
| ATOM | 2384 | O | HOH | W | 113 | 67.986 | 19.239 | 2.487 | 1.00 | 45.28 | O |
| ATOM | 2385 | O | HOH | W | 114 | 66.956 | 9.523 | −15.205 | 1.00 | 44.06 | O |
| ATOM | 2386 | O | HOH | W | 115 | 71.948 | 39.028 | 3.510 | 1.00 | 48.92 | O |
| ATOM | 2387 | O | HOH | W | 116 | 73.384 | 36.583 | 9.395 | 1.00 | 49.46 | O |
| ATOM | 2388 | O | HOH | W | 117 | 69.213 | 13.943 | 11.885 | 1.00 | 43.79 | O |
| ATOM | 2389 | O | HOH | W | 118 | 92.376 | 29.991 | −13.421 | 1.00 | 50.66 | O |
| ATOM | 2390 | O | HOH | W | 119 | 71.748 | 33.616 | 8.364 | 1.00 | 44.60 | O |
| ATOM | 2391 | O | HOH | W | 120 | 72.751 | 30.625 | 9.138 | 1.00 | 54.54 | O |
| ATOM | 2392 | O | HOH | W | 121 | 44.373 | 14.896 | 1.763 | 1.00 | 54.90 | O |
| ATOM | 2393 | O | HOH | W | 122 | 72.331 | 37.663 | 5.252 | 1.00 | 54.91 | O |
| ATOM | 2394 | O | HOH | W | 123 | 85.766 | 37.929 | 7.966 | 1.00 | 45.42 | O |
| ATOM | 2395 | O | HOH | W | 124 | 82.375 | 46.624 | −12.952 | 1.00 | 49.98 | O |
| ATOM | 2396 | O | HOH | W | 125 | 69.185 | 5.514 | −10.117 | 1.00 | 57.15 | O |
| ATOM | 2397 | O | HOH | W | 126 | 72.843 | 16.943 | −2.415 | 1.00 | 48.35 | O |
| ATOM | 2398 | O | HOH | W | 127 | 58.459 | 18.549 | −11.193 | 1.00 | 68.47 | O |
| ATOM | 2399 | O | HOH | W | 128 | 64.272 | 33.293 | −12.839 | 1.00 | 48.86 | O |
| ATOM | 2400 | O | HOH | W | 129 | 59.782 | 37.121 | −16.253 | 1.00 | 59.29 | O |

[image] TABLE 4
REMARK Written by DEALPDB Version 1.13 (06/02)
REMARK Thu Jan 23 14:56:07 2003

| | | |
|---|---|---|
| HEADER | ---- | XX-XXX-XX xxxx |
| COMPND | --- | |

| REMARK | 3 | | |
|---|---|---|---|
| REMARK | 3 | REFINEMENT. | |
| REMARK | 3 | PROGRAM | REFMAC 5.1.25 |
| REMARK | 3 | AUTHORS | MURSHUDOV, VAGIN, DODSON |
| REMARK | 3 | | |

| | | | |
|---|---|---|---|
| REMARK | 3 | REFINEMENT TARGET | MAXIMUM LIKELIHOOD |
| REMARK | 3 | | |

| | | | |
|---|---|---|---|
| REMARK | 3 | DATA USED IN REFINEMENT. | |
| REMARK | 3 | RESOLUTION RANGE HIGH (ANGSTROMS) | 1.80 |
| REMARK | 3 | RESOLUTION RANGE LOW (ANGSTROMS) | 81.65 |
| REMARK | 3 | DATA CUTOFF (SIGMA(F)) | NONE |
| REMARK | 3 | COMPLETENESS FOR RANGE (%) | 99.77 |
| REMARK | 3 | NUMBER OF REFLECTIONS | 24820 |
| REMARK | 3 | | |
| REMARK | 3 | FIT TO DATA USED IN REFINEMENT. | |
| REMARK | 3 | CROSS-VALIDATION METHOD | THROUGHOUT |
| REMARK | 3 | FREE R VALUE TEST SET SELECTION | RANDOM |
| REMARK | 3 | R VALUE (WORKING + TEST SET) | 0.18829 |
| REMARK | 3 | R VALUE (WORKING SET) | 0.18620 |
| REMARK | 3 | FREE R VALUE | 0.22809 |
| REMARK | 3 | FREE R VALUE TEST SET SIZE (%) | 5.1 |
| REMARK | 3 | FREE R VALUE TEST SET COUNT | 1327 |
| REMARK | 3 | | |
| REMARK | 3 | FIT IN THE HIGHEST RESOLUTION BIN. | |
| REMARK | 3 | TOTAL NUMBER OF BINS USED | 20 |
| REMARK | 3 | BIN RESOLUTION RANGE HIGH | 1.800 |
| REMARK | 3 | BIN RESOLUTION RANGE LOW | 1.847 |
| REMARK | 3 | REFLECTION IN BIN (WORKING SET) | 1749 |
| REMARK | 3 | BIN R VALUE (WORKING SET) | 0.242 |
| REMARK | 3 | BIN FREE R VALUE SET COUNT | 90 |
| REMARK | 3 | BIN FREE R VALUE | 0.288 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | 2507 |
| REMARK | 3 | | |
| REMARK | 3 | B VALUES. | |

| | | | |
|---|---|---|---|
| REMARK | 3 | FROM WILSON PLOT (A**2) | NULL |
| REMARK | 3 | MEAN B VALUE (OVERALL, A**2) | 17.218 |

| | | |
|---|---|---|
| REMARK | 3 | OVERALL ANISOTROPIC B VALUE. |

| | | | |
|---|---|---|---|
| REMARK | 3 | B11 (A**2) | −0.09 |
| REMARK | 3 | B22 (A**2) | 0.14 |
| REMARK | 3 | B33 (A**2) | −0.04 |
| REMARK | 3 | B12 (A**2) | 0.00 |
| REMARK | 3 | B13 (A**2) | −0.02 |
| REMARK | 3 | B23 (A**2) | 0.00 |
| REMARK | 3 | | |

| | | | |
|---|---|---|---|
| REMARK | 3 | ESTIMATED OVERALL COORDINATE ERROR. | |
| REMARK | 3 | ESU BASED ON R VALUE (A) | 0.141 |
| REMARK | 3 | ESU BASED ON FREE R VALUE (A) | 0.133 |
| REMARK | 3 | ESU BASED ON MAXIMUM LIKELIHOOD (A) | 0.082 |
| REMARK | 3 | ESU FOR B VALUES BASED ON MAXIMUM LIKELIHOOD (A**2) | 2.620 |
| REMARK | 3 | | |

| | | | |
|---|---|---|---|
| REMARK | 3 | CORRELATION COEFFICIENTS. | |
| REMARK | 3 | CORRELATION COEFFICIENT FO-FC | 0.948 |
| REMARK | 3 | CORRELATION COEFFICIENT FO-FC FREE | 0.929 |
| REMARK | 3 | | |

| | | | | | |
|---|---|---|---|---|---|
| REMARK | 3 | RMS DEVIATIONS FROM IDEAL VALUES | COUNT | RMS | WEIGHT |
| REMARK | 3 | BOND LENGTHS REFINED ATOMS (A) | 2310 | 0.010 | 0.022 |
| REMARK | 3 | BOND LENGTHS OTHERS (A) | 2097 | 0.002 | 0.020 |
| REMARK | 3 | BOND ANGLES REFINED ATOMS (DEGREES) | 3134 | 1.372 | 1.981 |
| REMARK | 3 | BOND ANGLES OTHERS (DEGREES) | 4890 | 0.790 | 3.000 |
| REMARK | 3 | TORSION ANGLES, PERIOD 1 (DEGREES) | 272 | 5.281 | 5.000 |
| REMARK | 3 | CHIRAL-CENTER RESTRAINTS (A**3) | 344 | 0.076 | 0.200 |
| REMARK | 3 | GENERAL PLANES REFINED ATOMS (A) | 2489 | 0.005 | 0.020 |
| REMARK | 3 | GENERAL PLANES OTHERS (A) | 465 | 0.002 | 0.020 |
| REMARK | 3 | NON-BONDED CONTACTS REFINED ATOMS (A) | 475 | 0.205 | 0.200 |
| REMARK | 3 | NON-BONDED CONTACTS OTHERS (A) | 2364 | 0.223 | 0.200 |
| REMARK | 3 | NON-BONDED TORSION OTHERS (A) | 1222 | 0.081 | 0.200 |
| REMARK | 3 | H-BOND (X...Y) REFINED ATOMS (A) | 147 | 0.162 | 0.200 |
| REMARK | 3 | SYMMETRY VDW REFINED ATOMS (A) | 21 | 0.168 | 0.200 |
| REMARK | 3 | SYMMETRY VDW OTHERS (A) | 86 | 0.250 | 0.200 |
| REMARK | 3 | SYMMETRY H-BOND REFINED ATOMS (A) | 14 | 0.111 | 0.200 |
| REMARK | 3 | | | | |
| REMARK | 3 | ISOTROPIC THERMAL FACTOR RESTRAINTS. | COUNT | RMS | WEIGHT |
| REMARK | 3 | MAIN-CHAIN BOND REFINED ATOMS (A**2) | 1365 | 0.818 | 1.500 |
| REMARK | 3 | MAIN-CHAIN ANGLE REFINED ATOMS (A**2) | 2224 | 1.568 | 2.000 |
| REMARK | 3 | SIDE-CHAIN BOND REFINED ATOMS (A**2) | 945 | 2.206 | 3.000 |
| REMARK | 3 | SIDE-CHAIN ANGLE REFINED ATOMS (A**2) | 910 | 3.668 | 4.500 |
| REMARK | 3 | | | | |

| | | | |
|---|---|---|---|
| REMARK | 3 | NCS RESTRAINTS STATISTICS | |
| REMARK | 3 | NUMBER OF NCS GROUPS | NULL |
| REMARK | 3 | | |
| REMARK | 3 | | |
| REMARK | 3 | TLS DETAILS | |
| REMARK | 3 | NUMBER OF TLS GROUPS | 1 |
| REMARK | 3 | | |
| REMARK | 3 | TLS GROUP | 1 |

| | | | |
|---|---|---|---|
| REMARK | 3 | NUMBER OF COMPONENTS GROUP | 1 |

| | | |
|---|---|---|
| REMARK | 3 | COMPONENTS C SSSEQI TO C SSSEQI |
| REMARK | 3 | RESIDUE RANGE A 419 A 691 |
| REMARK | 3 | ORIGIN FOR THE GROUP (A) 6.9620 1.7680 19.1340 |
| REMARK | 3 | T TENSOR |

| | | | | | |
|---|---|---|---|---|---|
| REMARK | 3 | T11 | 0.0048 | T22 | 0.0352 |
| REMARK | 3 | T33 | 0.0580 | T12 | −0.0119 |
| REMARK | 3 | T13 | −0.0081 | T23 | 0.0084 |

| | | |
|---|---|---|
| REMARK | 3 | L TENSOR |

| | | | | | |
|---|---|---|---|---|---|
| REMARK | 3 | L11 | 0.3962 | L22 | 0.3784 |
| REMARK | 3 | L33 | 0.2902 | L12 | −0.1647 |
| REMARK | 3 | L13 | 0.0731 | L23 | 0.0592 |

| | | |
|---|---|---|
| REMARK | 3 | S TENSOR |

| | | | | | | | |
|---|---|---|---|---|---|---|---|
| REMARK | 3 | S11 | −0.0145 | S12 | 0.0246 | S13 | 0.0170 |
| REMARK | 3 | S21 | 0.0077 | S22 | 0.0410 | S23 | 0.0381 |
| REMARK | 3 | S31 | −0.0159 | S32 | 0.0355 | S33 | −0.0265 |
| REMARK | 3 | | | | | | |
| REMARK | 3 | | | | | | |

| REMARK | 3 | BULK SOLVENT MODELLING. | |
|---|---|---|---|
| REMARK | 3 | METHOD USED | BABINET MODEL WITH MASK |

| REMARK | 3 | PARAMETERS FOR MASK CALCULATION | |
|---|---|---|---|
| REMARK | 3 | VDW PROBE RADIUS | 1.40 |
| REMARK | 3 | ION PROBE RADIUS | 0.80 |
| REMARK | 3 | SHRINKAGE RADIUS | 0.80 |
| REMARK | 3 | | |

| | | |
|---|---|---|
| REMARK | 3 | OTHER REFINEMENT REMARKS |
| REMARK | 3 | HYDROGENS HAVE BEEN ADDED IN THE RIDING POSITIONS |
| REMARK | 3 | |

| CRYST1 | 37.316 46.978 81.109 90.00 92.83 90.00 P 1 21 1 | | | | | | | | | | | |
|---|---|---|---|---|---|---|---|---|---|---|---|---|
| SCALE1 | 0.026798 0.000000 0.001323 0.00000 | | | | | | | | | | | |
| SCALE2 | 0.000000 0.021287 0.000000 0.00000 | | | | | | | | | | | |
| SCALE3 | 0.000000 0.000000 0.012344 0.00000 | | | | | | | | | | | |
| ATOM | 1 | N | MET | A | 419 | −17.724 | 15.274 | 26.545 | 1.00 | 41.92 | A | N |
| ATOM | 3 | CA | MET | A | 419 | −16.798 | 15.014 | 25.404 | 1.00 | 41.87 | A | C |
| ATOM | 5 | CB | MET | A | 419 | −17.513 | 15.303 | 24.075 | 1.00 | 42.37 | A | C |
| ATOM | 8 | CG | MET | A | 419 | −18.905 | 14.692 | 23.955 | 1.00 | 44.21 | A | C |
| ATOM | 11 | SD | MET | A | 419 | −18.872 | 12.884 | 23.783 | 1.00 | 48.64 | A | S |
| ATOM | 12 | CE | MET | A | 419 | −19.036 | 12.354 | 25.521 | 1.00 | 47.82 | A | C |
| ATOM | 16 | C | MET | A | 419 | −15.527 | 15.875 | 25.505 | 1.00 | 40.85 | A | C |
| ATOM | 17 | O | MET | A | 419 | −14.857 | 16.115 | 24.495 | 1.00 | 41.39 | A | O |
| ATOM | 20 | N | ILE | A | 420 | −15.200 | 16.322 | 26.719 | 1.00 | 39.21 | A | N |
| ATOM | 22 | CA | ILE | A | 420 | −14.050 | 17.208 | 26.982 | 1.00 | 37.79 | A | C |
| ATOM | 24 | CB | ILE | A | 420 | −12.697 | 16.519 | 26.689 | 1.00 | 37.82 | A | C |
| ATOM | 26 | CG1 | ILE | A | 420 | −12.557 | 15.240 | 27.512 | 1.00 | 38.41 | A | C |
| ATOM | 29 | CD1 | ILE | A | 420 | −11.209 | 14.556 | 27.348 | 1.00 | 38.81 | A | C |
| ATOM | 33 | CG2 | ILE | A | 420 | −11.539 | 17.494 | 26.996 | 1.00 | 37.64 | A | C |
| ATOM | 37 | C | ILE | A | 420 | −14.081 | 18.526 | 26.218 | 1.00 | 36.12 | A | C |
| ATOM | 38 | O | ILE | A | 420 | −13.850 | 18.561 | 25.013 | 1.00 | 36.25 | A | O |
| ATOM | 39 | N | ALA | A | 421 | −14.316 | 19.613 | 26.935 | 1.00 | 34.13 | A | N |
| ATOM | 41 | CA | ALA | A | 421 | −14.207 | 20.936 | 26.356 | 1.00 | 32.57 | A | C |
| ATOM | 43 | CB | ALA | A | 421 | −15.126 | 21.909 | 27.076 | 1.00 | 32.58 | A | C |
| ATOM | 47 | C | ALA | A | 421 | −12.762 | 21.394 | 26.457 | 1.00 | 31.11 | A | C |
| ATOM | 48 | O | ALA | A | 421 | −12.009 | 20.935 | 27.315 | 1.00 | 30.48 | A | O |
| ATOM | 49 | N | ARG | A | 422 | −12.385 | 22.305 | 25.572 | 1.00 | 29.35 | A | N |
| ATOM | 51 | CA | ARG | A | 422 | −11.069 | 22.917 | 25.610 | 1.00 | 28.21 | A | C |
| ATOM | 53 | CB | ARG | A | 422 | −10.957 | 23.974 | 24.506 | 1.00 | 28.08 | A | C |
| ATOM | 56 | CG | ARG | A | 422 | −9.542 | 24.501 | 24.279 | 1.00 | 27.30 | A | C |
| ATOM | 59 | CD | ARG | A | 422 | −9.471 | 25.640 | 23.289 | 1.00 | 25.94 | A | C |
| ATOM | 62 | NE | ARG | A | 422 | −10.069 | 25.288 | 22.005 | 1.00 | 25.59 | A | N |
| ATOM | 64 | CZ | ARG | A | 422 | −9.474 | 24.572 | 21.057 | 1.00 | 24.52 | A | C |
| ATOM | 65 | NH1 | ARG | A | 422 | −8.241 | 24.095 | 21.225 | 1.00 | 22.64 | A | N |
| ATOM | 68 | NH2 | ARG | A | 422 | −10.124 | 24.320 | 19.932 | 1.00 | 24.29 | A | N |
| ATOM | 71 | C | ARG | A | 422 | −10.773 | 23.535 | 26.985 | 1.00 | 27.22 | A | C |
| ATOM | 72 | O | ARG | A | 422 | −9.632 | 23.519 | 27.435 | 1.00 | 26.74 | A | O |
| ATOM | 73 | N | GLU | A | 423 | −11.808 | 24.051 | 27.652 | 1.00 | 26.08 | A | N |
| ATOM | 75 | CA | GLU | A | 423 | −11.674 | 24.666 | 28.979 | 1.00 | 25.78 | A | C |
| ATOM | 77 | CB | GLU | A | 423 | −13.012 | 25.237 | 29.474 | 1.00 | 26.29 | A | C |
| ATOM | 80 | CG | GLU | A | 423 | −13.662 | 26.233 | 28.552 | 1.00 | 28.01 | A | C |
| ATOM | 83 | CD | GLU | A | 423 | −14.629 | 25.584 | 27.583 | 1.00 | 29.62 | A | C |
| ATOM | 84 | OE1 | GLU | A | 423 | −14.183 | 25.287 | 26.450 | 1.00 | 28.40 | A | O |
| ATOM | 85 | OE2 | GLU | A | 423 | −15.823 | 25.382 | 27.960 | 1.00 | 30.82 | A | O |
| ATOM | 86 | C | GLU | A | 423 | −11.224 | 23.675 | 30.040 | 1.00 | 24.55 | A | C |
| ATOM | 87 | O | GLU | A | 423 | −10.636 | 24.070 | 31.034 | 1.00 | 24.25 | A | O |
| ATOM | 88 | N | ASP | A | 424 | −11.550 | 22.401 | 29.843 | 1.00 | 23.65 | A | N |
| ATOM | 90 | CA | ASP | A | 424 | −11.151 | 21.351 | 30.778 | 1.00 | 23.18 | A | C |
| ATOM | 92 | CB | ASP | A | 424 | −11.925 | 20.056 | 30.503 | 1.00 | 23.13 | A | C |
| ATOM | 95 | CG | ASP | A | 424 | −13.436 | 20.219 | 30.670 | 1.00 | 25.17 | A | C |
| ATOM | 96 | OD1 | ASP | A | 424 | −13.848 | 21.127 | 31.427 | 1.00 | 26.20 | A | O |
| ATOM | 97 | OD2 | ASP | A | 424 | −14.276 | 19.481 | 30.095 | 1.00 | 26.16 | A | O |
| ATOM | 98 | C | ASP | A | 424 | −9.639 | 21.067 | 30.742 | 1.00 | 22.42 | A | C |
| ATOM | 99 | O | ASP | A | 424 | −9.148 | 20.360 | 31.606 | 1.00 | 21.99 | A | O |
| ATOM | 100 | N | VAL | A | 425 | −8.920 | 21.606 | 29.752 | 1.00 | 21.44 | A | N |
| ATOM | 102 | CA | VAL | A | 425 | −7.488 | 21.342 | 29.592 | 1.00 | 21.23 | A | C |
| ATOM | 104 | CB | VAL | A | 425 | −7.184 | 20.639 | 28.249 | 1.00 | 21.09 | A | C |
| ATOM | 106 | CG1 | VAL | A | 425 | −5.678 | 20.393 | 28.092 | 1.00 | 21.37 | A | C |
| ATOM | 110 | CG2 | VAL | A | 425 | −7.963 | 19.337 | 28.133 | 1.00 | 20.93 | A | C |
| ATOM | 114 | C | VAL | A | 425 | −6.715 | 22.641 | 29.649 | 1.00 | 21.24 | A | C |
| ATOM | 115 | O | VAL | A | 425 | −6.957 | 23.541 | 28.836 | 1.00 | 21.03 | A | O |
| ATOM | 116 | N | VAL | A | 426 | −5.824 | 22.742 | 30.631 | 1.00 | 20.75 | A | N |
| ATOM | 118 | CA | VAL | A | 426 | −4.965 | 23.894 | 30.830 | 1.00 | 21.32 | A | C |
| ATOM | 120 | CB | VAL | A | 426 | −4.992 | 24.367 | 32.300 | 1.00 | 21.30 | A | C |
| ATOM | 122 | CG1 | VAL | A | 426 | −4.044 | 25.545 | 32.514 | 1.00 | 22.52 | A | C |
| ATOM | 126 | CG2 | VAL | A | 426 | −6.415 | 24.743 | 32.718 | 1.00 | 21.78 | A | C |
| ATOM | 130 | C | VAL | A | 426 | −3.522 | 23.530 | 30.466 | 1.00 | 21.27 | A | C |
| ATOM | 131 | O | VAL | A | 426 | −2.931 | 22.621 | 31.046 | 1.00 | 20.65 | A | O |
| ATOM | 132 | N | LEU | A | 427 | −2.960 | 24.253 | 29.509 | 1.00 | 21.37 | A | N |
| ATOM | 134 | CA | LEU | A | 427 | −1.585 | 24.024 | 29.079 | 1.00 | 21.38 | A | C |
| ATOM | 136 | CB | LEU | A | 427 | −1.413 | 24.387 | 27.593 | 1.00 | 21.39 | A | C |
| ATOM | 139 | CG | LEU | A | 427 | −2.428 | 23.797 | 26.603 | 1.00 | 21.09 | A | C |
| ATOM | 141 | CD1 | LEU | A | 427 | −2.214 | 24.341 | 25.191 | 1.00 | 21.00 | A | C |
| ATOM | 145 | CD2 | LEU | A | 427 | −2.399 | 22.267 | 26.592 | 1.00 | 20.75 | A | C |
| ATOM | 149 | C | LEU | A | 427 | −0.626 | 24.841 | 29.931 | 1.00 | 22.15 | A | C |
| ATOM | 150 | O | LEU | A | 427 | −0.819 | 26.043 | 30.102 | 1.00 | 21.62 | A | O |
| ATOM | 151 | N | ASN | A | 428 | 0.413 | 24.189 | 30.448 | 1.00 | 22.43 | A | N |
| ATOM | 153 | CA | ASN | A | 428 | 1.416 | 24.834 | 31.311 | 1.00 | 23.45 | A | C |
| ATOM | 155 | CB | ASN | A | 428 | 1.710 | 23.923 | 32.508 | 1.00 | 23.76 | A | C |
| ATOM | 158 | CG | ASN | A | 428 | 0.458 | 23.579 | 33.293 | 1.00 | 26.13 | A | C |
| ATOM | 159 | OD1 | ASN | A | 428 | 0.301 | 22.454 | 33.774 | 1.00 | 30.61 | A | O |
| ATOM | 160 | ND2 | ASN | A | 428 | −0.455 | 24.536 | 33.400 | 1.00 | 28.03 | A | N |
| ATOM | 163 | C | ASN | A | 428 | 2.728 | 25.192 | 30.611 | 1.00 | 23.29 | A | C |
| ATOM | 164 | O | ASN | A | 428 | 3.316 | 26.231 | 30.907 | 1.00 | 23.11 | A | O |
| ATOM | 165 | N | ARG | A | 429 | 3.217 | 24.316 | 29.732 | 1.00 | 23.23 | A | N |
| ATOM | 167 | CA | ARG | A | 429 | 4.438 | 24.593 | 28.965 | 1.00 | 23.80 | A | C |
| ATOM | 169 | CB | ARG | A | 429 | 5.678 | 24.419 | 29.846 | 1.00 | 24.47 | A | C |
| ATOM | 172 | CG | ARG | A | 429 | 5.945 | 22.999 | 30.298 | 1.00 | 26.49 | A | C |
| ATOM | 175 | CD | ARG | A | 429 | 7.254 | 22.845 | 31.078 | 1.00 | 30.54 | A | C |
| ATOM | 178 | NE | ARG | A | 429 | 7.866 | 21.544 | 30.824 | 1.00 | 34.08 | A | N |
| ATOM | 180 | CZ | ARG | A | 429 | 8.873 | 21.315 | 29.980 | 1.00 | 35.96 | A | C |
| ATOM | 181 | NH1 | ARG | A | 429 | 9.438 | 22.308 | 29.290 | 1.00 | 36.94 | A | N |
| ATOM | 184 | NH2 | ARG | A | 429 | 9.330 | 20.077 | 29.838 | 1.00 | 36.82 | A | N |
| ATOM | 187 | C | ARG | A | 429 | 4.596 | 23.731 | 27.715 | 1.00 | 23.63 | A | C |
| ATOM | 188 | O | ARG | A | 429 | 3.847 | 22.785 | 27.506 | 1.00 | 22.57 | A | O |
| ATOM | 189 | N | ILE | A | 430 | 5.592 | 24.053 | 26.895 | 1.00 | 23.59 | A | N |
| ATOM | 191 | CA | ILE | A | 430 | 5.901 | 23.258 | 25.711 | 1.00 | 24.18 | A | C |
| ATOM | 193 | CB | ILE | A | 430 | 6.382 | 24.161 | 24.535 | 1.00 | 24.28 | A | C |
| ATOM | 195 | CG1 | ILE | A | 430 | 5.211 | 25.021 | 24.046 | 1.00 | 24.59 | A | C |
| ATOM | 198 | CD1 | ILE | A | 430 | 5.513 | 25.900 | 22.844 | 1.00 | 23.92 | A | C |
| ATOM | 202 | CG2 | ILE | A | 430 | 6.967 | 23.305 | 23.398 | 1.00 | 24.51 | A | C |
| ATOM | 206 | C | ILE | A | 430 | 6.914 | 22.169 | 26.036 | 1.00 | 25.15 | A | C |
| ATOM | 207 | O | ILE | A | 430 | 7.978 | 22.435 | 26.594 | 1.00 | 25.52 | A | O |
| ATOM | 208 | N | LEU | A | 431 | 6.555 | 20.940 | 25.683 | 1.00 | 26.06 | A | N |
| ATOM | 210 | CA | LEU | A | 431 | 7.351 | 19.745 | 25.935 | 1.00 | 27.29 | A | C |
| ATOM | 212 | CB | LEU | A | 431 | 6.411 | 18.533 | 25.929 | 1.00 | 27.83 | A | C |
| ATOM | 215 | CG | LEU | A | 431 | 6.627 | 17.314 | 26.814 | 1.00 | 29.05 | A | C |
| ATOM | 217 | CD1 | LEU | A | 431 | 7.002 | 17.675 | 28.242 | 1.00 | 29.90 | A | C |
| ATOM | 221 | CD2 | LEU | A | 431 | 5.346 | 16.505 | 26.789 | 1.00 | 29.38 | A | C |
| ATOM | 225 | C | LEU | A | 431 | 8.423 | 19.572 | 24.863 | 1.00 | 28.18 | A | C |
| ATOM | 226 | O | LEU | A | 431 | 9.582 | 19.243 | 25.149 | 1.00 | 28.38 | A | O |
| ATOM | 227 | N | GLY | A | 432 | 8.024 | 19.793 | 23.620 | 1.00 | 28.92 | A | N |
| ATOM | 229 | CA | GLY | A | 432 | 8.932 | 19.691 | 22.500 | 1.00 | 29.66 | A | C |
| ATOM | 232 | C | GLY | A | 432 | 8.267 | 19.963 | 21.166 | 1.00 | 30.32 | A | C |
| ATOM | 233 | O | GLY | A | 432 | 7.040 | 20.023 | 21.065 | 1.00 | 29.86 | A | O |
| ATOM | 234 | N | GLU | A | 433 | 9.096 | 20.144 | 20.144 | 1.00 | 31.32 | A | N |
| ATOM | 236 | CA | GLU | A | 433 | 8.636 | 20.260 | 18.767 | 1.00 | 32.42 | A | C |
| ATOM | 238 | CB | GLU | A | 433 | 9.600 | 21.120 | 17.946 | 1.00 | 33.03 | A | C |
| ATOM | 241 | CG | GLU | A | 433 | 9.426 | 21.056 | 16.433 | 1.00 | 35.97 | A | C |
| ATOM | 244 | CD | GLU | A | 433 | 8.136 | 21.696 | 15.966 | 1.00 | 39.52 | A | C |
| ATOM | 245 | OE1 | GLU | A | 433 | 7.078 | 21.062 | 16.142 | 1.00 | 41.45 | A | O |
| ATOM | 246 | OE2 | GLU | A | 433 | 8.178 | 22.828 | 15.417 | 1.00 | 43.45 | A | O |
| ATOM | 247 | C | GLU | A | 433 | 8.559 | 18.853 | 18.200 | 1.00 | 32.62 | A | C |
| ATOM | 248 | O | GLU | A | 433 | 9.584 | 18.218 | 17.959 | 1.00 | 33.07 | A | O |
| ATOM | 249 | N | GLY | A | 434 | 7.341 | 18.364 | 18.027 | 1.00 | 32.46 | A | N |
| ATOM | 251 | CA | GLY | A | 434 | 7.110 | 17.080 | 17.411 | 1.00 | 32.53 | A | C |
| ATOM | 254 | C | GLY | A | 434 | 6.926 | 17.141 | 15.904 | 1.00 | 32.66 | A | C |
| ATOM | 255 | O | GLY | A | 434 | 7.033 | 18.193 | 15.266 | 1.00 | 32.19 | A | O |
| ATOM | 256 | N | PHE | A | 435 | 6.619 | 15.974 | 15.353 | 1.00 | 32.83 | A | N |
| ATOM | 258 | CA | PHE | A | 435 | 6.413 | 15.765 | 13.926 | 1.00 | 32.68 | A | C |
| ATOM | 260 | CB | PHE | A | 435 | 6.032 | 14.294 | 13.703 | 1.00 | 33.30 | A | C |
| ATOM | 263 | CG | PHE | A | 435 | 6.016 | 13.890 | 12.267 | 1.00 | 35.29 | A | C |
| ATOM | 264 | CD1 | PHE | A | 435 | 7.202 | 13.628 | 11.601 | 1.00 | 37.64 | A | C |
| ATOM | 266 | CE1 | PHE | A | 435 | 7.197 | 13.260 | 10.263 | 1.00 | 38.62 | A | C |
| ATOM | 268 | CZ | PHE | A | 435 | 6.000 | 13.162 | 9.582 | 1.00 | 38.93 | A | C |
| ATOM | 270 | CE2 | PHE | A | 435 | 4.807 | 13.434 | 10.236 | 1.00 | 38.98 | A | C |
| ATOM | 272 | CD2 | PHE | A | 435 | 4.821 | 13.794 | 11.575 | 1.00 | 36.99 | A | C |
| ATOM | 274 | C | PHE | A | 435 | 5.330 | 16.662 | 13.319 | 1.00 | 31.77 | A | C |
| ATOM | 275 | O | PHE | A | 435 | 5.533 | 17.269 | 12.262 | 1.00 | 31.44 | A | O |
| ATOM | 276 | N | PHE | A | 436 | 4.178 | 16.734 | 13.982 | 1.00 | 30.64 | A | N |
| ATOM | 278 | CA | PHE | A | 436 | 3.030 | 17.476 | 13.465 | 1.00 | 29.83 | A | C |
| ATOM | 280 | CB | PHE | A | 436 | 1.716 | 16.792 | 13.880 | 1.00 | 30.65 | A | C |
| ATOM | 283 | CG | PHE | A | 436 | 1.425 | 15.511 | 13.114 | 1.00 | 33.59 | A | C |
| ATOM | 284 | CD1 | PHE | A | 436 | 0.993 | 15.554 | 11.791 | 1.00 | 36.89 | A | C |
| ATOM | 286 | CE1 | PHE | A | 436 | 0.731 | 14.368 | 11.076 | 1.00 | 38.35 | A | C |
| ATOM | 288 | CZ | PHE | A | 436 | 0.917 | 13.136 | 11.692 | 1.00 | 38.06 | A | C |
| ATOM | 290 | CE2 | PHE | A | 436 | 1.354 | 13.086 | 13.006 | 1.00 | 37.74 | A | C |
| ATOM | 292 | CD2 | PHE | A | 436 | 1.608 | 14.268 | 13.709 | 1.00 | 36.81 | A | C |
| ATOM | 294 | C | PHE | A | 436 | 3.036 | 18.949 | 13.904 | 1.00 | 27.92 | A | C |
| ATOM | 295 | O | PHE | A | 436 | 2.435 | 19.783 | 13.242 | 1.00 | 26.90 | A | O |
| ATOM | 296 | N | GLY | A | 437 | 3.713 | 19.242 | 15.021 | 1.00 | 25.85 | A | N |
| ATOM | 298 | CA | GLY | A | 437 | 3.773 | 20.582 | 15.598 | 1.00 | 24.35 | A | C |
| ATOM | 301 | C | GLY | A | 437 | 4.152 | 20.555 | 17.076 | 1.00 | 23.03 | A | C |
| ATOM | 302 | O | GLY | A | 437 | 4.635 | 19.549 | 17.596 | 1.00 | 22.52 | A | O |
| ATOM | 303 | N | GLU | A | 438 | 3.912 | 21.660 | 17.774 | 1.00 | 21.56 | A | N |
| ATOM | 305 | CA | GLU | A | 438 | 4.295 | 21.761 | 19.176 | 1.00 | 20.35 | A | C |
| ATOM | 307 | CB | GLU | A | 438 | 4.114 | 23.191 | 19.694 | 1.00 | 20.82 | A | C |
| ATOM | 310 | CG | GLU | A | 438 | 4.921 | 24.235 | 18.922 | 1.00 | 23.31 | A | C |
| ATOM | 313 | CD | GLU | A | 438 | 6.325 | 24.443 | 19.459 | 1.00 | 26.75 | A | C |
| ATOM | 314 | OE1 | GLU | A | 438 | 6.960 | 23.454 | 19.896 | 1.00 | 24.49 | A | O |
| ATOM | 315 | OE2 | GLU | A | 438 | 6.792 | 25.611 | 19.446 | 1.00 | 29.56 | A | O |
| ATOM | 316 | C | GLU | A | 438 | 3.473 | 20.781 | 20.016 | 1.00 | 18.49 | A | C |
| ATOM | 317 | O | GLU | A | 438 | 2.306 | 20.552 | 19.744 | 1.00 | 17.45 | A | O |
| ATOM | 318 | N | VAL | A | 439 | 4.124 | 20.199 | 21.017 | 1.00 | 17.29 | A | N |
| ATOM | 320 | CA | VAL | A | 439 | 3.498 | 19.315 | 21.986 | 1.00 | 16.28 | A | C |
| ATOM | 322 | CB | VAL | A | 439 | 4.183 | 17.933 | 22.024 | 1.00 | 16.57 | A | C |
| ATOM | 324 | CG1 | VAL | A | 439 | 3.516 | 17.038 | 23.065 | 1.00 | 17.25 | A | C |
| ATOM | 328 | CG2 | VAL | A | 439 | 4.141 | 17.301 | 20.643 | 1.00 | 16.55 | A | C |
| ATOM | 332 | C | VAL | A | 439 | 3.623 | 19.974 | 23.350 | 1.00 | 15.68 | A | C |
| ATOM | 333 | O | VAL | A | 439 | 4.705 | 20.380 | 23.732 | 1.00 | 14.39 | A | O |
| ATOM | 334 | N | TYR | A | 440 | 2.508 | 20.084 | 24.068 | 1.00 | 14.79 | A | N |
| ATOM | 336 | CA | TYR | A | 440 | 2.447 | 20.789 | 25.345 | 1.00 | 14.71 | A | C |
| ATOM | 338 | CB | TYR | A | 440 | 1.269 | 21.749 | 25.334 | 1.00 | 14.65 | A | C |
| ATOM | 341 | CG | TYR | A | 440 | 1.348 | 22.842 | 24.301 | 1.00 | 15.25 | A | C |
| ATOM | 342 | CD1 | TYR | A | 440 | 1.809 | 24.110 | 24.639 | 1.00 | 16.36 | A | C |
| ATOM | 344 | CE1 | TYR | A | 440 | 1.860 | 25.135 | 23.691 | 1.00 | 16.17 | A | C |
| ATOM | 346 | CZ | TYR | A | 440 | 1.436 | 24.895 | 22.395 | 1.00 | 18.28 | A | C |
| ATOM | 347 | OH | TYR | A | 440 | 1.490 | 25.905 | 21.453 | 1.00 | 20.25 | A | O |
| ATOM | 349 | CE2 | TYR | A | 440 | 0.967 | 23.644 | 22.038 | 1.00 | 16.17 | A | C |
| ATOM | 351 | CD2 | TYR | A | 440 | 0.916 | 22.630 | 22.994 | 1.00 | 14.61 | A | C |
| ATOM | 353 | C | TYR | A | 440 | 2.228 | 19.817 | 26.479 | 1.00 | 15.34 | A | C |
| ATOM | 354 | O | TYR | A | 440 | 1.608 | 18.765 | 26.270 | 1.00 | 15.00 | A | O |
| ATOM | 355 | N | GLU | A | 441 | 2.723 | 20.168 | 27.669 | 1.00 | 15.04 | A | N |
| ATOM | 357 | CA | GLU | A | 441 | 2.340 | 19.514 | 28.921 | 1.00 | 16.43 | A | C |
| ATOM | 359 | CB | GLU | A | 441 | 3.513 | 19.470 | 29.916 | 1.00 | 17.43 | A | C |
| ATOM | 362 | CG | GLU | A | 441 | 3.244 | 18.575 | 31.117 | 1.00 | 21.96 | A | C |
| ATOM | 365 | CD | GLU | A | 441 | 4.331 | 18.631 | 32.173 | 1.00 | 28.06 | A | C |
| ATOM | 366 | OE1 | GLU | A | 441 | 5.533 | 18.478 | 31.828 | 1.00 | 31.85 | A | O |
| ATOM | 367 | OE2 | GLU | A | 441 | 3.975 | 18.829 | 33.361 | 1.00 | 32.28 | A | O |
| ATOM | 368 | C | GLU | A | 441 | 1.196 | 20.317 | 29.514 | 1.00 | 15.91 | A | C |
| ATOM | 369 | O | GLU | A | 441 | 1.205 | 21.549 | 29.457 | 1.00 | 15.73 | A | O |
| ATOM | 370 | N | GLY | A | 442 | 0.214 | 19.627 | 30.074 | 1.00 | 15.38 | A | N |
| ATOM | 372 | CA | GLY | A | 442 | −0.938 | 20.279 | 30.679 | 1.00 | 15.56 | A | C |
| ATOM | 375 | C | GLY | A | 442 | −1.686 | 19.408 | 31.671 | 1.00 | 15.46 | A | C |
| ATOM | 376 | O | GLY | A | 442 | −1.266 | 18.295 | 31.998 | 1.00 | 14.91 | A | O |
| ATOM | 377 | N | VAL | A | 443 | −2.799 | 19.937 | 32.159 | 1.00 | 15.90 | A | N |
| ATOM | 379 | CA | VAL | A | 443 | −3.655 | 19.250 | 33.120 | 1.00 | 15.95 | A | C |
| ATOM | 381 | CB | VAL | A | 443 | −3.585 | 19.926 | 34.515 | 1.00 | 15.99 | A | C |
| ATOM | 383 | CG1 | VAL | A | 443 | −4.535 | 19.256 | 35.486 | 1.00 | 16.92 | A | C |
| ATOM | 387 | CG2 | VAL | A | 443 | −2.159 | 19.901 | 35.054 | 1.00 | 16.66 | A | C |
| ATOM | 391 | C | VAL | A | 443 | −5.088 | 19.235 | 32.606 | 1.00 | 16.11 | A | C |
| ATOM | 392 | O | VAL | A | 443 | −5.677 | 20.280 | 32.328 | 1.00 | 16.39 | A | O |
| ATOM | 393 | N | TYR | A | 444 | −5.645 | 18.039 | 32.474 | 1.00 | 16.07 | A | N |
| ATOM | 395 | CA | TYR | A | 444 | −7.053 | 17.833 | 32.189 | 1.00 | 16.30 | A | C |
| ATOM | 397 | CB | TYR | A | 444 | −7.210 | 16.696 | 31.179 | 1.00 | 16.55 | A | C |
| ATOM | 400 | CG | TYR | A | 444 | −8.608 | 16.106 | 31.085 | 1.00 | 16.49 | A | C |
| ATOM | 401 | CD1 | TYR | A | 444 | −9.735 | 16.925 | 30.992 | 1.00 | 17.40 | A | C |
| ATOM | 403 | CE1 | TYR | A | 444 | −11.002 | 16.376 | 30.907 | 1.00 | 18.98 | A | C |
| ATOM | 405 | CZ | TYR | A | 444 | −11.159 | 15.013 | 30.900 | 1.00 | 19.67 | A | C |
| ATOM | 406 | OH | TYR | A | 444 | −12.417 | 14.454 | 30.818 | 1.00 | 22.12 | A | O |
| ATOM | 408 | CE2 | TYR | A | 444 | −10.066 | 14.189 | 30.992 | 1.00 | 18.71 | A | C |
| ATOM | 410 | CD2 | TYR | A | 444 | −8.801 | 14.737 | 31.070 | 1.00 | 18.30 | A | C |
| ATOM | 412 | C | TYR | A | 444 | −7.828 | 17.530 | 33.495 | 1.00 | 16.91 | A | C |
| ATOM | 413 | O | TYR | A | 444 | −7.500 | 16.593 | 34.223 | 1.00 | 16.06 | A | O |
| ATOM | 414 | N | THR | A | 445 | −8.845 | 18.338 | 33.779 | 1.00 | 17.53 | A | N |
| ATOM | 416 | CA | THR | A | 445 | −9.703 | 18.146 | 34.944 | 1.00 | 18.31 | A | C |
| ATOM | 418 | CB | THR | A | 445 | −9.911 | 19.475 | 35.684 | 1.00 | 18.17 | A | C |
| ATOM | 420 | OG1 | THR | A | 445 | −8.651 | 20.045 | 36.030 | 1.00 | 19.23 | A | O |
| ATOM | 422 | CG2 | THR | A | 445 | −10.578 | 19.262 | 37.044 | 1.00 | 18.29 | A | C |
| ATOM | 426 | C | THR | A | 445 | −11.032 | 17.632 | 34.430 | 1.00 | 19.18 | A | C |
| ATOM | 427 | O | THR | A | 445 | −11.702 | 18.345 | 33.694 | 1.00 | 19.17 | A | O |
| ATOM | 428 | N | ASN | A | 446 | −11.402 | 16.403 | 34.791 | 1.00 | 19.93 | A | N |
| ATOM | 430 | CA | ASN | A | 446 | −12.665 | 15.825 | 34.351 | 1.00 | 21.09 | A | C |
| ATOM | 432 | CB | ASN | A | 446 | −12.617 | 14.266 | 34.318 | 1.00 | 21.05 | A | C |
| ATOM | 435 | CG | ASN | A | 446 | −12.502 | 13.602 | 35.704 | 1.00 | 21.28 | A | C |
| ATOM | 436 | OD1 | ASN | A | 446 | −12.158 | 12.407 | 35.796 | 1.00 | 22.39 | A | O |
| ATOM | 437 | ND2 | ASN | A | 446 | −12.778 | 14.345 | 36.766 | 1.00 | 18.07 | A | N |
| ATOM | 440 | C | ASN | A | 446 | −13.831 | 16.414 | 35.163 | 1.00 | 22.07 | A | C |
| ATOM | 441 | O | ASN | A | 446 | −13.646 | 17.396 | 35.907 | 1.00 | 22.06 | A | O |
| ATOM | 442 | N | HIS | A | 447 | −15.025 | 15.861 | 34.999 | 1.00 | 23.22 | A | N |
| ATOM | 444 | CA | HIS | A | 447 | −16.215 | 16.446 | 35.621 | 1.00 | 24.56 | A | C |
| ATOM | 446 | CB | HIS | A | 447 | −17.479 | 16.009 | 34.878 | 1.00 | 25.52 | A | C |
| ATOM | 449 | CG | HIS | A | 447 | −17.560 | 16.535 | 33.474 | 1.00 | 28.90 | A | C |
| ATOM | 450 | ND1 | HIS | A | 447 | −17.485 | 17.879 | 33.177 | 1.00 | 32.01 | A | N |
| ATOM | 452 | CE1 | HIS | A | 447 | −17.581 | 18.047 | 31.869 | 1.00 | 32.90 | A | C |
| ATOM | 454 | NE2 | HIS | A | 447 | −17.712 | 16.859 | 31.306 | 1.00 | 33.63 | A | N |
| ATOM | 456 | CD2 | HIS | A | 447 | −17.703 | 15.896 | 32.289 | 1.00 | 32.36 | A | C |
| ATOM | 458 | C | HIS | A | 447 | −16.323 | 16.121 | 37.113 | 1.00 | 24.17 | A | C |
| ATOM | 459 | O | HIS | A | 447 | −17.141 | 16.715 | 37.819 | 1.00 | 24.60 | A | O |
| ATOM | 460 | N | LYS | A | 448 | −15.513 | 15.168 | 37.575 | 1.00 | 23.56 | A | N |
| ATOM | 462 | CA | LYS | A | 448 | −15.440 | 14.802 | 38.994 | 1.00 | 23.13 | A | C |
| ATOM | 464 | CB | LYS | A | 448 | −15.188 | 13.302 | 39.137 | 1.00 | 23.02 | A | C |
| ATOM | 467 | CG | LYS | A | 448 | −16.354 | 12.461 | 38.679 | 1.00 | 23.56 | A | C |
| ATOM | 470 | CD | LYS | A | 448 | −15.916 | 11.043 | 38.364 | 1.00 | 24.68 | A | C |
| ATOM | 473 | CE | LYS | A | 448 | −17.090 | 10.180 | 37.907 | 1.00 | 25.53 | A | C |
| ATOM | 476 | NZ | LYS | A | 448 | −16.689 | 8.742 | 37.931 | 1.00 | 23.92 | A | N |
| ATOM | 480 | C | LYS | A | 448 | −14.353 | 15.561 | 39.758 | 1.00 | 22.72 | A | C |
| ATOM | 481 | O | LYS | A | 448 | −14.197 | 15.369 | 40.974 | 1.00 | 23.18 | A | O |
| ATOM | 482 | N | GLY | A | 449 | −13.606 | 16.401 | 39.048 | 1.00 | 21.47 | A | N |
| ATOM | 484 | CA | GLY | A | 449 | −12.580 | 17.234 | 39.648 | 1.00 | 21.53 | A | C |
| ATOM | 487 | C | GLY | A | 449 | −11.237 | 16.542 | 39.729 | 1.00 | 21.19 | A | C |
| ATOM | 488 | O | GLY | A | 449 | −10.319 | 17.047 | 40.366 | 1.00 | 20.74 | A | O |
| ATOM | 489 | N | GLU | A | 450 | −11.127 | 15.386 | 39.080 | 1.00 | 20.98 | A | N |
| ATOM | 491 | CA | GLU | A | 450 | −9.879 | 14.630 | 39.054 | 1.00 | 21.35 | A | C |
| ATOM | 493 | CB | GLU | A | 450 | −10.150 | 13.172 | 38.691 | 1.00 | 21.63 | A | C |
| ATOM | 496 | CG | GLU | A | 450 | −11.186 | 12.486 | 39.565 | 1.00 | 23.33 | A | C |
| ATOM | 499 | CD | GLU | A | 450 | −11.347 | 11.024 | 39.214 | 1.00 | 26.08 | A | C |
| ATOM | 500 | OE1 | GLU | A | 450 | −10.821 | 10.178 | 39.963 | 1.00 | 26.94 | A | O |
| ATOM | 501 | OE2 | GLU | A | 450 | −12.024 | 10.726 | 38.201 | 1.00 | 28.51 | A | O |
| ATOM | 502 | C | GLU | A | 450 | −8.955 | 15.232 | 38.019 | 1.00 | 21.24 | A | C |
| ATOM | 503 | O | GLU | A | 450 | −9.376 | 15.478 | 36.902 | 1.00 | 20.28 | A | O |
| ATOM | 504 | N | LYS | A | 451 | −7.691 | 15.436 | 38.388 | 1.00 | 21.76 | A | N |
| ATOM | 506 | CA | LYS | A | 451 | −6.694 | 16.059 | 37.507 | 1.00 | 21.92 | A | C |
| ATOM | 508 | CB | LYS | A | 451 | −5.963 | 17.166 | 38.254 | 1.00 | 22.51 | A | C |
| ATOM | 511 | CG | LYS | A | 451 | −6.919 | 18.230 | 38.784 | 1.00 | 23.99 | A | C |
| ATOM | 514 | CD | LYS | A | 451 | −6.239 | 19.546 | 39.068 | 1.00 | 26.30 | A | C |
| ATOM | 517 | CE | LYS | A | 451 | −7.250 | 20.582 | 39.558 | 1.00 | 27.02 | A | C |
| ATOM | 520 | NZ | LYS | A | 451 | −7.947 | 21.286 | 38.459 | 1.00 | 26.84 | A | N |
| ATOM | 524 | C | LYS | A | 451 | −5.707 | 15.026 | 36.956 | 1.00 | 21.70 | A | C |
| ATOM | 525 | O | LYS | A | 451 | −5.111 | 14.249 | 37.707 | 1.00 | 22.12 | A | O |
| ATOM | 526 | N | ILE | A | 452 | −5.586 | 15.005 | 35.633 | 1.00 | 20.88 | A | N |
| ATOM | 528 | CA | ILE | A | 452 | −4.729 | 14.074 | 34.923 | 1.00 | 20.89 | A | C |
| ATOM | 530 | CB | ILE | A | 452 | −5.587 | 13.104 | 34.051 | 1.00 | 21.84 | A | C |
| ATOM | 532 | CG1 | ILE | A | 452 | −4.773 | 11.937 | 33.512 | 1.00 | 24.27 | A | C |
| ATOM | 535 | CD1 | ILE | A | 452 | −4.557 | 10.839 | 34.539 | 1.00 | 27.03 | A | C |
| ATOM | 539 | CG2 | ILE | A | 452 | −6.222 | 13.783 | 32.894 | 1.00 | 23.92 | A | C |
| ATOM | 543 | C | ILE | A | 452 | −3.743 | 14.883 | 34.086 | 1.00 | 19.23 | A | C |
| ATOM | 544 | O | ILE | A | 452 | −4.127 | 15.801 | 33.366 | 1.00 | 17.19 | A | O |
| ATOM | 545 | N | ASN | A | 453 | −2.468 | 14.561 | 34.227 | 1.00 | 18.03 | A | N |
| ATOM | 547 | CA | ASN | A | 453 | −1.427 | 15.173 | 33.421 | 1.00 | 16.96 | A | C |
| ATOM | 549 | CB | ASN | A | 453 | −0.039 | 14.893 | 34.019 | 1.00 | 17.63 | A | C |
| ATOM | 552 | CG | ASN | A | 453 | 0.144 | 15.546 | 35.375 | 1.00 | 19.82 | A | C |
| ATOM | 553 | OD1 | ASN | A | 453 | −0.283 | 16.675 | 35.581 | 1.00 | 20.59 | A | O |
| ATOM | 554 | ND2 | ASN | A | 453 | 0.782 | 14.835 | 36.307 | 1.00 | 22.47 | A | N |
| ATOM | 557 | C | ASN | A | 453 | −1.537 | 14.640 | 32.002 | 1.00 | 15.39 | A | C |
| ATOM | 558 | O | ASN | A | 453 | −1.741 | 13.432 | 31.792 | 1.00 | 13.98 | A | O |
| ATOM | 559 | N | VAL | A | 454 | −1.442 | 15.553 | 31.040 | 1.00 | 13.24 | A | N |
| ATOM | 561 | CA | VAL | A | 454 | −1.609 | 15.230 | 29.630 | 1.00 | 12.28 | A | C |
| ATOM | 563 | CB | VAL | A | 454 | −3.001 | 15.671 | 29.107 | 1.00 | 11.15 | A | C |
| ATOM | 565 | CG1 | VAL | A | 454 | −4.107 | 14.882 | 29.798 | 1.00 | 11.44 | A | C |
| ATOM | 569 | CG2 | VAL | A | 454 | −3.210 | 17.193 | 29.273 | 1.00 | 10.74 | A | C |
| ATOM | 573 | C | VAL | A | 454 | −0.515 | 15.843 | 28.752 | 1.00 | 12.12 | A | C |
| ATOM | 574 | O | VAL | A | 454 | 0.128 | 16.843 | 29.123 | 1.00 | 11.68 | A | O |
| ATOM | 575 | N | ALA | A | 455 | −0.276 | 15.196 | 27.615 | 1.00 | 11.65 | A | N |
| ATOM | 577 | CA | ALA | A | 455 | 0.496 | 15.770 | 26.521 | 1.00 | 11.60 | A | C |
| ATOM | 579 | CB | ALA | A | 455 | 1.493 | 14.785 | 25.979 | 1.00 | 12.08 | A | C |
| ATOM | 583 | C | ALA | A | 455 | −0.511 | 16.142 | 25.435 | 1.00 | 12.46 | A | C |
| ATOM | 584 | O | ALA | A | 455 | −1.331 | 15.327 | 25.055 | 1.00 | 12.88 | A | O |
| ATOM | 585 | N | VAL | A | 456 | −0.459 | 17.384 | 24.968 | 1.00 | 11.82 | A | N |
| ATOM | 587 | CA | VAL | A | 456 | −1.368 | 17.858 | 23.960 | 1.00 | 12.46 | A | C |
| ATOM | 589 | CB | VAL | A | 456 | −2.073 | 19.125 | 24.456 | 1.00 | 12.62 | A | C |
| ATOM | 591 | CG1 | VAL | A | 456 | −2.956 | 19.704 | 23.364 | 1.00 | 13.07 | A | C |
| ATOM | 595 | CG2 | VAL | A | 456 | −2.883 | 18.786 | 25.679 | 1.00 | 12.74 | A | C |
| ATOM | 599 | C | VAL | A | 456 | −0.622 | 18.148 | 22.668 | 1.00 | 13.13 | A | C |
| ATOM | 600 | O | VAL | A | 456 | 0.252 | 19.006 | 22.631 | 1.00 | 12.69 | A | O |
| ATOM | 601 | N | LYS | A | 457 | −0.958 | 17.415 | 21.616 | 1.00 | 13.43 | A | N |
| ATOM | 603 | CA | LYS | A | 457 | −0.324 | 17.577 | 20.322 | 1.00 | 14.66 | A | C |
| ATOM | 605 | CB | LYS | A | 457 | −0.214 | 16.220 | 19.615 | 1.00 | 15.55 | A | C |
| ATOM | 608 | CG | LYS | A | 457 | 0.694 | 15.217 | 20.350 | 1.00 | 18.42 | A | C |
| ATOM | 611 | CD | LYS | A | 457 | 0.840 | 13.874 | 19.626 | 1.00 | 22.96 | A | C |
| ATOM | 614 | CE | LYS | A | 457 | 1.221 | 14.008 | 18.151 | 1.00 | 26.16 | A | C |
| ATOM | 617 | NZ | LYS | A | 457 | 1.738 | 12.702 | 17.608 | 1.00 | 29.52 | A | N |
| ATOM | 621 | C | LYS | A | 457 | −1.123 | 18.569 | 19.480 | 1.00 | 14.79 | A | C |
| ATOM | 622 | O | LYS | A | 457 | −2.350 | 18.553 | 19.483 | 1.00 | 14.94 | A | O |
| ATOM | 623 | N | THR | A | 458 | −0.421 | 19.452 | 18.787 | 1.00 | 16.01 | A | N |
| ATOM | 625 | CA | THR | A | 458 | −1.056 | 20.426 | 17.902 | 1.00 | 17.14 | A | C |
| ATOM | 627 | CB | THR | A | 458 | −0.974 | 21.856 | 18.470 | 1.00 | 17.23 | A | C |
| ATOM | 629 | OG1 | THR | A | 458 | 0.390 | 22.295 | 18.492 | 1.00 | 16.75 | A | O |
| ATOM | 631 | CG2 | THR | A | 458 | −1.437 | 21.907 | 19.927 | 1.00 | 18.01 | A | C |
| ATOM | 635 | C | THR | A | 458 | −0.380 | 20.398 | 16.541 | 1.00 | 18.40 | A | C |
| ATOM | 636 | O | THR | A | 458 | 0.705 | 19.846 | 16.397 | 1.00 | 18.01 | A | O |
| ATOM | 637 | N | CYS | A | 459 | −1.032 | 21.017 | 15.561 | 1.00 | 20.44 | A | N |
| ATOM | 639 | CA | CYS | A | 459 | −0.512 | 21.123 | 14.195 | 1.00 | 22.30 | A | C |
| ATOM | 641 | CB | CYS | A | 459 | −1.643 | 20.944 | 13.189 | 1.00 | 22.75 | A | C |
| ATOM | 644 | SG | CYS | A | 459 | −2.097 | 19.219 | 12.971 | 1.00 | 27.79 | A | S |
| ATOM | 645 | C | CYS | A | 459 | 0.174 | 22.470 | 13.959 | 1.00 | 22.93 | A | C |
| ATOM | 646 | O | CYS | A | 459 | −0.344 | 23.509 | 14.347 | 1.00 | 22.16 | A | O |
| ATOM | 647 | N | LYS | A | 460 | 1.347 | 22.426 | 13.337 | 1.00 | 24.00 | A | N |
| ATOM | 649 | CA | LYS | A | 460 | 2.082 | 23.624 | 12.970 | 1.00 | 25.15 | A | C |
| ATOM | 651 | CB | LYS | A | 460 | 3.487 | 23.276 | 12.461 | 1.00 | 25.42 | A | C |
| ATOM | 654 | CG | LYS | A | 460 | 3.540 | 22.407 | 11.195 | 1.00 | 27.60 | A | C |
| ATOM | 657 | CD | LYS | A | 460 | 4.982 | 22.089 | 10.797 | 1.00 | 31.10 | A | C |
| ATOM | 660 | CE | LYS | A | 460 | 5.641 | 21.091 | 11.756 | 1.00 | 33.59 | A | C |
| ATOM | 663 | NZ | LYS | A | 460 | 6.879 | 20.456 | 11.191 | 1.00 | 35.01 | A | N |
| ATOM | 667 | C | LYS | A | 460 | 1.301 | 24.390 | 11.917 | 1.00 | 25.65 | A | C |
| ATOM | 668 | O | LYS | A | 460 | 0.444 | 23.818 | 11.226 | 1.00 | 25.83 | A | O |
| ATOM | 669 | N | LYS | A | 461 | 1.574 | 25.687 | 11.808 | 1.00 | 25.80 | A | N |
| ATOM | 671 | CA | LYS | A | 461 | 0.826 | 26.538 | 10.882 | 1.00 | 26.62 | A | C |
| ATOM | 673 | CB | LYS | A | 461 | 1.252 | 28.006 | 11.008 | 1.00 | 26.88 | A | C |
| ATOM | 676 | CG | LYS | A | 461 | 2.713 | 28.253 | 10.821 | 1.00 | 27.10 | A | C |
| ATOM | 679 | CD | LYS | A | 461 | 2.998 | 29.749 | 10.885 | 1.00 | 26.75 | A | C |
| ATOM | 682 | CE | LYS | A | 461 | 4.299 | 30.069 | 10.215 | 1.00 | 26.14 | A | C |
| ATOM | 685 | NZ | LYS | A | 461 | 4.520 | 31.513 | 10.213 | 1.00 | 24.09 | A | N |
| ATOM | 689 | C | LYS | A | 461 | 0.920 | 26.065 | 9.424 | 1.00 | 26.93 | A | C |
| ATOM | 690 | O | LYS | A | 461 | −0.036 | 26.221 | 8.675 | 1.00 | 27.53 | A | O |
| ATOM | 691 | N | ASP | A | 462 | 2.046 | 25.462 | 9.044 | 1.00 | 27.62 | A | N |
| ATOM | 693 | CA | ASP | A | 462 | 2.260 | 24.961 | 7.678 | 1.00 | 28.08 | A | C |
| ATOM | 695 | CB | ASP | A | 462 | 3.747 | 25.062 | 7.309 | 1.00 | 28.77 | A | C |
| ATOM | 698 | CG | ASP | A | 462 | 4.025 | 24.701 | 5.853 | 1.00 | 30.67 | A | C |
| ATOM | 699 | OD1 | ASP | A | 462 | 3.273 | 25.156 | 4.964 | 1.00 | 33.53 | A | O |
| ATOM | 700 | OD2 | ASP | A | 462 | 4.973 | 23.959 | 5.506 | 1.00 | 34.82 | A | O |
| ATOM | 701 | C | ASP | A | 462 | 1.772 | 23.509 | 7.560 | 1.00 | 27.68 | A | C |
| ATOM | 702 | O | ASP | A | 462 | 2.542 | 22.601 | 7.211 | 1.00 | 28.33 | A | O |
| ATOM | 703 | N | CYS | A | 463 | 0.500 | 23.302 | 7.891 | 1.00 | 26.54 | A | N |
| ATOM | 705 | CA | CYS | A | 463 | −0.123 | 21.991 | 7.824 | 1.00 | 25.64 | A | C |
| ATOM | 707 | CB | CYS | A | 463 | −0.529 | 21.522 | 9.217 | 1.00 | 25.87 | A | C |
| ATOM | 710 | SG | CYS | A | 463 | −1.141 | 19.833 | 9.252 | 1.00 | 28.94 | A | S |
| ATOM | 711 | C | CYS | A | 463 | −1.350 | 22.104 | 6.918 | 1.00 | 23.92 | A | C |
| ATOM | 712 | O | CYS | A | 463 | −2.319 | 22.772 | 7.251 | 1.00 | 23.05 | A | O |
| ATOM | 713 | N | THR | A | 464 | −1.277 | 21.480 | 5.751 | 1.00 | 22.41 | A | N |
| ATOM | 715 | CA | THR | A | 464 | −2.385 | 21.500 | 4.800 | 1.00 | 21.51 | A | C |
| ATOM | 717 | CB | THR | A | 464 | −1.947 | 20.928 | 3.435 | 1.00 | 21.47 | A | C |
| ATOM | 719 | OG1 | THR | A | 464 | −1.405 | 19.607 | 3.600 | 1.00 | 19.22 | A | O |
| ATOM | 721 | CG2 | THR | A | 464 | −0.804 | 21.743 | 2.823 | 1.00 | 21.55 | A | C |
| ATOM | 725 | C | THR | A | 464 | −3.571 | 20.689 | 5.316 | 1.00 | 21.41 | A | C |
| ATOM | 726 | O | THR | A | 464 | −3.414 | 19.755 | 6.111 | 1.00 | 19.82 | A | O |
| ATOM | 727 | N | LEU | A | 465 | −4.758 | 21.040 | 4.832 | 1.00 | 21.68 | A | N |
| ATOM | 729 | CA | LEU | A | 465 | −5.969 | 20.261 | 5.090 | 1.00 | 22.10 | A | C |
| ATOM | 731 | CB | LEU | A | 465 | −7.165 | 20.906 | 4.378 | 1.00 | 22.26 | A | C |
| ATOM | 734 | CG | LEU | A | 465 | −7.631 | 22.229 | 4.986 | 1.00 | 22.45 | A | C |
| ATOM | 736 | CD1 | LEU | A | 465 | −8.763 | 22.808 | 4.170 | 1.00 | 24.00 | A | C |
| ATOM | 740 | CD2 | LEU | A | 465 | −8.079 | 22.028 | 6.446 | 1.00 | 23.54 | A | C |
| ATOM | 744 | C | LEU | A | 465 | −5.798 | 18.821 | 4.624 | 1.00 | 22.80 | A | C |
| ATOM | 745 | O | LEU | A | 465 | −6.322 | 17.892 | 5.234 | 1.00 | 22.69 | A | O |
| ATOM | 746 | N | ASP | A | 466 | −5.073 | 18.665 | 3.524 | 1.00 | 23.58 | A | N |
| ATOM | 748 | CA | ASP | A | 466 | −4.666 | 17.367 | 3.004 | 1.00 | 25.03 | A | C |
| ATOM | 750 | CB | ASP | A | 466 | −3.709 | 17.593 | 1.820 | 1.00 | 25.18 | A | C |
| ATOM | 753 | CG | ASP | A | 466 | −3.414 | 16.332 | 1.038 | 1.00 | 27.16 | A | C |
| ATOM | 754 | OD1 | ASP | A | 466 | −3.623 | 15.216 | 1.570 | 1.00 | 26.35 | A | O |
| ATOM | 755 | OD2 | ASP | A | 466 | −2.966 | 16.376 | −0.139 | 1.00 | 29.99 | A | O |
| ATOM | 756 | C | ASP | A | 466 | −3.993 | 16.539 | 4.101 | 1.00 | 25.88 | A | C |
| ATOM | 757 | O | ASP | A | 466 | −4.460 | 15.448 | 4.445 | 1.00 | 25.28 | A | O |
| ATOM | 758 | N | ASN | A | 467 | −2.900 | 17.063 | 4.656 | 1.00 | 27.10 | A | N |
| ATOM | 760 | CA | ASN | A | 467 | −2.161 | 16.350 | 5.702 | 1.00 | 28.05 | A | C |
| ATOM | 762 | CB | ASN | A | 467 | −0.772 | 16.985 | 5.916 | 1.00 | 28.48 | A | C |
| ATOM | 765 | CG | ASN | A | 467 | 0.134 | 16.866 | 4.684 | 1.00 | 29.52 | A | C |
| ATOM | 766 | OD1 | ASN | A | 467 | 0.992 | 17.713 | 4.447 | 1.00 | 31.12 | A | O |
| ATOM | 767 | ND2 | ASN | A | 467 | −0.059 | 15.816 | 3.901 | 1.00 | 33.05 | A | N |
| ATOM | 770 | C | ASN | A | 467 | −2.920 | 16.263 | 7.037 | 1.00 | 28.75 | A | C |
| ATOM | 771 | O | ASN | A | 467 | −2.724 | 15.322 | 7.806 | 1.00 | 28.72 | A | O |
| ATOM | 772 | N | LYS | A | 468 | −3.794 | 17.232 | 7.296 | 1.00 | 29.65 | A | N |
| ATOM | 774 | CA | LYS | A | 468 | −4.560 | 17.300 | 8.544 | 1.00 | 30.66 | A | C |
| ATOM | 776 | CB | LYS | A | 468 | −5.273 | 18.642 | 8.639 | 1.00 | 30.86 | A | C |
| ATOM | 779 | CG | LYS | A | 468 | −5.702 | 19.038 | 10.031 | 1.00 | 32.87 | A | C |
| ATOM | 782 | CD | LYS | A | 468 | −4.950 | 20.278 | 10.513 | 1.00 | 35.25 | A | C |
| ATOM | 785 | CE | LYS | A | 468 | −5.304 | 21.521 | 9.671 | 1.00 | 36.51 | A | C |
| ATOM | 788 | NZ | LYS | A | 468 | −4.860 | 22.805 | 10.300 | 1.00 | 37.11 | A | N |
| ATOM | 792 | C | LYS | A | 468 | −5.604 | 16.186 | 8.644 | 1.00 | 31.56 | A | C |
| ATOM | 793 | O | LYS | A | 468 | −5.905 | 15.716 | 9.740 | 1.00 | 31.30 | A | O |
| ATOM | 794 | N | GLU | A | 469 | −6.159 | 15.779 | 7.504 | 1.00 | 32.55 | A | N |
| ATOM | 796 | CA | GLU | A | 469 | −7.144 | 14.694 | 7.473 | 1.00 | 33.52 | A | C |
| ATOM | 798 | CB | GLU | A | 469 | −7.866 | 14.646 | 6.117 | 1.00 | 33.78 | A | C |
| ATOM | 801 | CG | GLU | A | 469 | −8.835 | 13.476 | 5.924 | 1.00 | 34.57 | A | C |
| ATOM | 804 | CD | GLU | A | 469 | −9.939 | 13.398 | 6.975 | 1.00 | 36.41 | A | C |
| ATOM | 805 | OE1 | GLU | A | 469 | −10.241 | 14.421 | 7.638 | 1.00 | 36.96 | A | O |
| ATOM | 806 | OE2 | GLU | A | 469 | −10.527 | 12.301 | 7.131 | 1.00 | 38.63 | A | O |
| ATOM | 807 | C | GLU | A | 469 | −6.471 | 13.355 | 7.789 | 1.00 | 34.36 | A | C |
| ATOM | 808 | O | GLU | A | 469 | −7.067 | 12.508 | 8.436 | 1.00 | 33.99 | A | O |
| ATOM | 809 | N | LYS | A | 470 | −5.226 | 13.185 | 7.344 | 1.00 | 35.36 | A | N |
| ATOM | 811 | CA | LYS | A | 470 | −4.441 | 11.983 | 7.651 | 1.00 | 36.27 | A | C |
| ATOM | 813 | CB | LYS | A | 470 | −3.087 | 12.022 | 6.942 | 1.00 | 36.53 | A | C |
| ATOM | 816 | CG | LYS | A | 470 | −3.155 | 12.204 | 5.444 | 1.00 | 37.48 | A | C |
| ATOM | 819 | CD | LYS | A | 470 | −1.766 | 12.076 | 4.833 | 1.00 | 38.99 | A | C |
| ATOM | 822 | CE | LYS | A | 470 | −1.822 | 11.889 | 3.334 | 1.00 | 39.11 | A | C |
| ATOM | 825 | NZ | LYS | A | 470 | −0.566 | 11.286 | 2.828 | 1.00 | 39.24 | A | N |
| ATOM | 829 | C | LYS | A | 470 | −4.184 | 11.839 | 9.144 | 1.00 | 36.64 | A | C |
| ATOM | 830 | O | LYS | A | 470 | −4.342 | 10.758 | 9.709 | 1.00 | 37.23 | A | O |
| ATOM | 831 | N | PHE | A | 471 | −3.773 | 12.938 | 9.763 | 1.00 | 36.96 | A | N |
| ATOM | 833 | CA | PHE | A | 471 | −3.512 | 13.023 | 11.201 | 1.00 | 37.25 | A | C |
| ATOM | 835 | CB | PHE | A | 471 | −2.955 | 14.420 | 11.517 | 1.00 | 37.75 | A | C |
| ATOM | 838 | CG | PHE | A | 471 | −2.489 | 14.615 | 12.947 | 1.00 | 40.27 | A | C |
| ATOM | 839 | CD1 | PHE | A | 471 | −1.834 | 13.605 | 13.650 | 1.00 | 42.26 | A | C |
| ATOM | 841 | CE1 | PHE | A | 471 | −1.407 | 13.815 | 14.958 | 1.00 | 43.12 | A | C |
| ATOM | 843 | CZ | PHE | A | 471 | −1.611 | 15.056 | 15.572 | 1.00 | 43.43 | A | C |
| ATOM | 845 | CE2 | PHE | A | 471 | −2.243 | 16.066 | 14.883 | 1.00 | 42.92 | A | C |
| ATOM | 847 | CD2 | PHE | A | 471 | −2.680 | 15.847 | 13.578 | 1.00 | 42.50 | A | C |
| ATOM | 849 | C | PHE | A | 471 | −4.770 | 12.764 | 12.031 | 1.00 | 36.80 | A | C |
| ATOM | 850 | O | PHE | A | 471 | −4.725 | 12.035 | 13.017 | 1.00 | 36.85 | A | O |
| ATOM | 851 | N | MET | A | 472 | −5.885 | 13.369 | 11.630 | 1.00 | 36.07 | A | N |
| ATOM | 853 | CA | MET | A | 472 | −7.154 | 13.199 | 12.338 | 1.00 | 36.08 | A | C |
| ATOM | 855 | CB | MET | A | 472 | −8.246 | 14.103 | 11.761 | 1.00 | 36.21 | A | C |
| ATOM | 858 | CG | MET | A | 472 | −8.083 | 15.565 | 12.091 | 1.00 | 38.47 | A | C |
| ATOM | 861 | SD | MET | A | 472 | −7.642 | 15.859 | 13.819 | 1.00 | 42.21 | A | S |
| ATOM | 862 | CE | MET | A | 472 | −5.860 | 15.966 | 13.721 | 1.00 | 42.25 | A | C |
| ATOM | 866 | C | MET | A | 472 | −7.626 | 11.758 | 12.262 | 1.00 | 35.10 | A | C |
| ATOM | 867 | O | MET | A | 472 | −8.011 | 11.176 | 13.278 | 1.00 | 34.68 | A | O |
| ATOM | 868 | N | SER | A | 473 | −7.599 | 11.200 | 11.056 | 1.00 | 34.38 | A | N |
| ATOM | 870 | CA | SER | A | 473 | −8.067 | 9.836 | 10.838 | 1.00 | 34.10 | A | C |
| ATOM | 872 | CB | SER | A | 473 | −8.111 | 9.481 | 9.341 | 1.00 | 34.17 | A | C |
| ATOM | 875 | OG | SER | A | 473 | −6.876 | 9.725 | 8.700 | 1.00 | 35.90 | A | O |
| ATOM | 877 | C | SER | A | 473 | −7.194 | 8.870 | 11.619 | 1.00 | 33.23 | A | C |
| ATOM | 878 | O | SER | A | 473 | −7.681 | 7.877 | 12.146 | 1.00 | 33.23 | A | O |
| ATOM | 879 | N | GLU | A | 474 | −5.909 | 9.196 | 11.728 | 1.00 | 32.29 | A | N |
| ATOM | 881 | CA | GLU | A | 474 | −4.957 | 8.405 | 12.504 | 1.00 | 31.53 | A | C |
| ATOM | 883 | CB | GLU | A | 474 | −3.532 | 8.889 | 12.235 | 1.00 | 32.06 | A | C |
| ATOM | 886 | CG | GLU | A | 474 | −2.462 | 8.083 | 12.940 | 1.00 | 35.27 | A | C |
| ATOM | 889 | CD | GLU | A | 474 | −1.059 | 8.482 | 12.520 | 1.00 | 38.82 | A | C |
| ATOM | 890 | OE1 | GLU | A | 474 | −0.125 | 7.684 | 12.765 | 1.00 | 41.03 | A | O |
| ATOM | 891 | OE2 | GLU | A | 474 | −0.896 | 9.590 | 11.945 | 1.00 | 41.72 | A | O |
| ATOM | 892 | C | GLU | A | 474 | −5.252 | 8.474 | 13.999 | 1.00 | 29.52 | A | C |
| ATOM | 893 | O | GLU | A | 474 | −5.194 | 7.463 | 14.689 | 1.00 | 28.59 | A | O |
| ATOM | 894 | N | ALA | A | 475 | −5.583 | 9.664 | 14.486 | 1.00 | 27.61 | A | N |
| ATOM | 896 | CA | ALA | A | 475 | −5.852 | 9.876 | 15.908 | 1.00 | 26.39 | A | C |
| ATOM | 898 | CB | ALA | A | 475 | −5.991 | 11.361 | 16.196 | 1.00 | 26.28 | A | C |
| ATOM | 902 | C | ALA | A | 475 | −7.117 | 9.144 | 16.379 | 1.00 | 25.41 | A | C |
| ATOM | 903 | O | ALA | A | 475 | −7.186 | 8.717 | 17.521 | 1.00 | 24.68 | A | O |
| ATOM | 904 | N | VAL | A | 476 | −8.111 | 9.024 | 15.501 | 1.00 | 24.57 | A | N |
| ATOM | 906 | CA | VAL | A | 476 | −9.364 | 8.342 | 15.837 | 1.00 | 24.02 | A | C |
| ATOM | 908 | CB | VAL | A | 476 | −10.457 | 8.593 | 14.764 | 1.00 | 24.14 | A | C |
| ATOM | 910 | CG1 | VAL | A | 476 | −11.668 | 7.695 | 14.975 | 1.00 | 24.34 | A | C |
| ATOM | 914 | CG2 | VAL | A | 476 | −10.910 | 10.051 | 14.786 | 1.00 | 24.76 | A | C |
| ATOM | 918 | C | VAL | A | 476 | −9.096 | 6.840 | 16.010 | 1.00 | 23.50 | A | C |
| ATOM | 919 | O | VAL | A | 476 | −9.673 | 6.207 | 16.889 | 1.00 | 22.85 | A | O |
| ATOM | 920 | N | ILE | A | 477 | −8.191 | 6.283 | 15.197 | 1.00 | 23.32 | A | N |
| ATOM | 922 | CA | ILE | A | 477 | −7.794 | 4.880 | 15.366 | 1.00 | 23.29 | A | C |
| ATOM | 924 | CB | ILE | A | 477 | −6.857 | 4.392 | 14.232 | 1.00 | 23.54 | A | C |
| ATOM | 926 | CG1 | ILE | A | 477 | −7.579 | 4.420 | 12.883 | 1.00 | 25.55 | A | C |
| ATOM | 929 | CD1 | ILE | A | 477 | −6.693 | 4.070 | 11.676 | 1.00 | 26.07 | A | C |
| ATOM | 933 | CG2 | ILE | A | 477 | −6.357 | 2.978 | 14.550 | 1.00 | 24.90 | A | C |
| ATOM | 937 | C | ILE | A | 477 | −7.126 | 4.693 | 16.729 | 1.00 | 22.19 | A | C |
| ATOM | 938 | O | ILE | A | 477 | −7.505 | 3.833 | 17.502 | 1.00 | 20.75 | A | O |
| ATOM | 939 | N | MET | A | 478 | −6.126 | 5.518 | 17.025 | 1.00 | 22.05 | A | N |
| ATOM | 941 | CA | MET | A | 478 | −5.479 | 5.507 | 18.341 | 1.00 | 21.67 | A | C |
| ATOM | 943 | CB | MET | A | 478 | −4.401 | 6.603 | 18.421 | 1.00 | 22.21 | A | C |
| ATOM | 946 | CG | MET | A | 478 | −3.172 | 6.370 | 17.537 | 1.00 | 23.35 | A | C |
| ATOM | 949 | SD | MET | A | 478 | −2.324 | 4.761 | 17.784 | 1.00 | 27.04 | A | S |
| ATOM | 950 | CE | MET | A | 478 | −1.687 | 4.917 | 19.390 | 1.00 | 24.74 | A | C |
| ATOM | 954 | C | MET | A | 478 | −6.463 | 5.660 | 19.508 | 1.00 | 21.22 | A | C |
| ATOM | 955 | O | MET | A | 478 | −6.280 | 5.042 | 20.541 | 1.00 | 20.80 | A | O |
| ATOM | 956 | N | LYS | A | 479 | −7.517 | 6.461 | 19.336 | 1.00 | 21.22 | A | N |
| ATOM | 958 | CA | LYS | A | 479 | −8.521 | 6.681 | 20.381 | 1.00 | 21.07 | A | C |
| ATOM | 960 | CB | LYS | A | 479 | −9.550 | 7.727 | 19.910 | 1.00 | 21.83 | A | C |
| ATOM | 963 | CG | LYS | A | 479 | −10.728 | 7.940 | 20.864 | 1.00 | 25.29 | A | C |
| ATOM | 966 | CD | LYS | A | 479 | −11.616 | 9.074 | 20.401 | 1.00 | 28.79 | A | C |
| ATOM | 969 | CE | LYS | A | 479 | −12.585 | 9.512 | 21.494 | 1.00 | 30.50 | A | C |
| ATOM | 972 | NZ | LYS | A | 479 | −13.545 | 8.454 | 21.868 | 1.00 | 31.90 | A | N |
| ATOM | 976 | C | LYS | A | 479 | −9.251 | 5.394 | 20.752 | 1.00 | 20.32 | A | C |
| ATOM | 977 | O | LYS | A | 479 | −9.653 | 5.195 | 21.906 | 1.00 | 20.37 | A | O |
| ATOM | 978 | N | ASN | A | 480 | −9.447 | 4.529 | 19.761 | 1.00 | 18.51 | A | N |
| ATOM | 980 | CA | ASN | A | 480 | −10.168 | 3.284 | 19.980 | 1.00 | 18.01 | A | C |
| ATOM | 982 | CB | ASN | A | 480 | −10.851 | 2.841 | 18.692 | 1.00 | 17.94 | A | C |
| ATOM | 985 | CG | ASN | A | 480 | −12.098 | 3.625 | 18.410 | 1.00 | 16.43 | A | C |
| ATOM | 986 | OD1 | ASN | A | 480 | −13.135 | 3.388 | 19.038 | 1.00 | 14.27 | A | O |
| ATOM | 987 | ND2 | ASN | A | 480 | −12.016 | 4.574 | 17.481 | 1.00 | 13.64 | A | N |
| ATOM | 990 | C | ASN | A | 480 | −9.277 | 2.174 | 20.512 | 1.00 | 17.93 | A | C |
| ATOM | 991 | O | ASN | A | 480 | −9.768 | 1.196 | 21.049 | 1.00 | 17.90 | A | O |
| ATOM | 992 | N | LEU | A | 481 | −7.969 | 2.305 | 20.345 | 1.00 | 18.54 | A | N |
| ATOM | 994 | CA | LEU | A | 481 | −7.052 | 1.344 | 20.933 | 1.00 | 19.58 | A | C |
| ATOM | 996 | CB | LEU | A | 481 | −5.647 | 1.510 | 20.375 | 1.00 | 19.65 | A | C |
| ATOM | 999 | CG | LEU | A | 481 | −5.418 | 0.995 | 18.958 | 1.00 | 19.49 | A | C |
| ATOM | 1001 | CD1 | LEU | A | 481 | −4.028 | 1.362 | 18.492 | 1.00 | 20.75 | A | C |
| ATOM | 1005 | CD2 | LEU | A | 481 | −5.645 | −0.497 | 18.891 | 1.00 | 19.12 | A | C |
| ATOM | 1009 | C | LEU | A | 481 | −7.028 | 1.560 | 22.430 | 1.00 | 20.27 | A | C |
| ATOM | 1010 | O | LEU | A | 481 | −6.658 | 2.630 | 22.899 | 1.00 | 22.42 | A | O |
| ATOM | 1011 | N | ASP | A | 482 | −7.411 | 0.543 | 23.177 | 1.00 | 20.28 | A | N |
| ATOM | 1013 | CA | ASP | A | 482 | −7.360 | 0.583 | 24.626 | 1.00 | 20.20 | A | C |
| ATOM | 1015 | CB | ASP | A | 482 | −8.781 | 0.626 | 25.196 | 1.00 | 21.36 | A | C |
| ATOM | 1018 | CG | ASP | A | 482 | −8.815 | 0.831 | 26.702 | 1.00 | 24.17 | A | C |
| ATOM | 1019 | OD1 | ASP | A | 482 | −9.901 | 0.603 | 27.288 | 1.00 | 31.84 | A | O |
| ATOM | 1020 | OD2 | ASP | A | 482 | −7.843 | 1.228 | 27.382 | 1.00 | 28.34 | A | O |
| ATOM | 1021 | C | ASP | A | 482 | −6.625 | −0.664 | 25.080 | 1.00 | 19.09 | A | C |
| ATOM | 1022 | O | ASP | A | 482 | −7.178 | −1.765 | 25.073 | 1.00 | 19.88 | A | O |
| ATOM | 1023 | N | HIS | A | 483 | −5.349 | −0.490 | 25.413 | 1.00 | 16.36 | A | N |
| ATOM | 1025 | CA | HIS | A | 483 | −4.508 | −1.572 | 25.890 | 1.00 | 15.18 | A | C |
| ATOM | 1027 | CB | HIS | A | 483 | −3.723 | −2.178 | 24.727 | 1.00 | 14.01 | A | C |
| ATOM | 1030 | CG | HIS | A | 483 | −3.068 | −3.474 | 25.069 | 1.00 | 13.88 | A | C |
| ATOM | 1031 | ND1 | HIS | A | 483 | −1.872 | −3.542 | 25.755 | 1.00 | 13.01 | A | N |
| ATOM | 1033 | CE1 | HIS | A | 483 | −1.569 | −4.812 | 25.969 | 1.00 | 13.34 | A | C |
| ATOM | 1035 | NE2 | HIS | A | 483 | −2.515 | −5.566 | 25.429 | 1.00 | 12.59 | A | N |
| ATOM | 1037 | CD2 | HIS | A | 483 | −3.465 | −4.752 | 24.867 | 1.00 | 12.02 | A | C |
| ATOM | 1039 | C | HIS | A | 483 | −3.553 | −1.015 | 26.964 | 1.00 | 14.57 | A | C |
| ATOM | 1040 | O | HIS | A | 483 | −3.092 | 0.118 | 26.840 | 1.00 | 14.09 | A | O |
| ATOM | 1041 | N | PRO | A | 484 | −3.278 | −1.775 | 28.023 | 1.00 | 14.32 | A | N |
| ATOM | 1042 | CA | PRO | A | 484 | −2.360 | −1.310 | 29.080 | 1.00 | 13.52 | A | C |
| ATOM | 1044 | CB | PRO | A | 484 | −2.254 | −2.519 | 30.037 | 1.00 | 14.18 | A | C |
| ATOM | 1047 | CG | PRO | A | 484 | −3.355 | −3.429 | 29.700 | 1.00 | 15.62 | A | C |
| ATOM | 1050 | CD | PRO | A | 484 | −3.866 | −3.091 | 28.339 | 1.00 | 14.94 | A | C |
| ATOM | 1053 | C | PRO | A | 484 | −0.956 | −0.924 | 28.610 | 1.00 | 12.37 | A | C |
| ATOM | 1054 | O | PRO | A | 484 | −0.285 | −0.173 | 29.308 | 1.00 | 12.09 | A | O |
| ATOM | 1055 | N | HIS | A | 485 | −0.532 | −1.423 | 27.448 | 1.00 | 11.71 | A | N |
| ATOM | 1057 | CA | HIS | A | 485 | 0.790 | −1.148 | 26.915 | 1.00 | 10.90 | A | C |
| ATOM | 1059 | CB | HIS | A | 485 | 1.606 | −2.438 | 26.948 | 1.00 | 11.48 | A | C |
| ATOM | 1062 | CG | HIS | A | 485 | 1.687 | −2.999 | 28.326 | 1.00 | 11.67 | A | C |
| ATOM | 1063 | ND1 | HIS | A | 485 | 2.263 | −2.293 | 29.357 | 1.00 | 10.00 | A | N |
| ATOM | 1065 | CE1 | HIS | A | 485 | 2.139 | −2.984 | 30.478 | 1.00 | 13.83 | A | C |
| ATOM | 1067 | NE2 | HIS | A | 485 | 1.496 | −4.105 | 30.211 | 1.00 | 13.41 | A | N |
| ATOM | 1069 | CD2 | HIS | A | 485 | 1.179 | −4.129 | 28.873 | 1.00 | 13.59 | A | C |
| ATOM | 1071 | C | HIS | A | 485 | 0.786 | −0.514 | 25.549 | 1.00 | 10.49 | A | C |
| ATOM | 1072 | O | HIS | A | 485 | 1.722 | −0.706 | 24.795 | 1.00 | 9.78 | A | O |
| ATOM | 1073 | N | ILE | A | 486 | −0.267 | 0.258 | 25.260 | 1.00 | 9.75 | A | N |
| ATOM | 1075 | CA | ILE | A | 486 | −0.313 | 1.165 | 24.107 | 1.00 | 9.72 | A | C |
| ATOM | 1077 | CB | ILE | A | 486 | −1.352 | 0.675 | 23.077 | 1.00 | 9.49 | A | C |
| ATOM | 1079 | CG1 | ILE | A | 486 | −0.941 | −0.698 | 22.509 | 1.00 | 9.88 | A | C |
| ATOM | 1082 | CD1 | ILE | A | 486 | −1.944 | −1.282 | 21.569 | 1.00 | 11.12 | A | C |
| ATOM | 1086 | CG2 | ILE | A | 486 | −1.516 | 1.670 | 21.927 | 1.00 | 10.54 | A | C |
| ATOM | 1090 | C | ILE | A | 486 | −0.683 | 2.561 | 24.617 | 1.00 | 10.03 | A | C |
| ATOM | 1091 | O | ILE | A | 486 | −1.500 | 2.684 | 25.533 | 1.00 | 9.12 | A | O |
| ATOM | 1092 | N | VAL | A | 487 | −0.079 | 3.585 | 24.034 | 1.00 | 10.75 | A | N |
| ATOM | 1094 | CA | VAL | A | 487 | −0.294 | 4.958 | 24.477 | 1.00 | 11.70 | A | C |
| ATOM | 1096 | CB | VAL | A | 487 | 0.535 | 6.007 | 23.674 | 1.00 | 11.46 | A | C |
| ATOM | 1098 | CG1 | VAL | A | 487 | 2.006 | 5.849 | 23.946 | 1.00 | 11.31 | A | C |
| ATOM | 1102 | CG2 | VAL | A | 487 | 0.242 | 5.925 | 22.175 | 1.00 | 12.24 | A | C |
| ATOM | 1106 | C | VAL | A | 487 | −1.782 | 5.278 | 24.394 | 1.00 | 12.42 | A | C |
| ATOM | 1107 | O | VAL | A | 487 | −2.479 | 4.824 | 23.481 | 1.00 | 10.87 | A | O |
| ATOM | 1108 | N | LYS | A | 488 | −2.269 | 6.010 | 25.386 | 1.00 | 14.04 | A | N |
| ATOM | 1110 | CA | LYS | A | 488 | −3.699 | 6.252 | 25.531 | 1.00 | 15.40 | A | C |
| ATOM | 1112 | CB | LYS | A | 488 | −4.147 | 6.081 | 26.979 | 1.00 | 16.29 | A | C |
| ATOM | 1115 | CG | LYS | A | 488 | −5.648 | 6.355 | 27.161 | 1.00 | 18.92 | A | C |
| ATOM | 1118 | CD | LYS | A | 488 | −6.109 | 6.153 | 28.588 | 1.00 | 22.97 | A | C |
| ATOM | 1121 | CE | LYS | A | 488 | −7.638 | 6.269 | 28.693 | 1.00 | 26.06 | A | C |
| ATOM | 1124 | NZ | LYS | A | 488 | −8.179 | 5.734 | 29.994 | 1.00 | 28.52 | A | N |
| ATOM | 1128 | C | LYS | A | 488 | −4.019 | 7.663 | 25.090 | 1.00 | 16.61 | A | C |
| ATOM | 1129 | O | LYS | A | 488 | −3.470 | 8.625 | 25.650 | 1.00 | 15.54 | A | O |
| ATOM | 1130 | N | LEU | A | 489 | −4.879 | 7.760 | 24.079 | 1.00 | 18.15 | A | N |
| ATOM | 1132 | CA | LEU | A | 489 | −5.458 | 9.028 | 23.644 | 1.00 | 20.47 | A | C |
| ATOM | 1134 | CB | LEU | A | 489 | −5.707 | 9.035 | 22.135 | 1.00 | 20.96 | A | C |
| ATOM | 1137 | CG | LEU | A | 489 | −6.318 | 10.312 | 21.519 | 1.00 | 22.08 | A | C |
| ATOM | 1139 | CD1 | LEU | A | 489 | −6.157 | 10.316 | 20.034 | 1.00 | 23.52 | A | C |
| ATOM | 1143 | CD2 | LEU | A | 489 | −7.776 | 10.472 | 21.876 | 1.00 | 24.93 | A | C |
| ATOM | 1147 | C | LEU | A | 489 | −6.734 | 9.229 | 24.426 | 1.00 | 22.11 | A | C |
| ATOM | 1148 | O | LEU | A | 489 | −7.625 | 8.370 | 24.402 | 1.00 | 23.38 | A | O |
| ATOM | 1149 | N | ILE | A | 490 | −6.813 | 10.361 | 25.125 | 1.00 | 22.70 | A | N |
| ATOM | 1151 | CA | ILE | A | 490 | −7.920 | 10.685 | 26.016 | 1.00 | 23.88 | A | C |
| ATOM | 1153 | CB | ILE | A | 490 | −7.400 | 11.539 | 27.185 | 1.00 | 24.15 | A | C |
| ATOM | 1155 | CG1 | ILE | A | 490 | −6.437 | 10.714 | 28.049 | 1.00 | 25.63 | A | C |
| ATOM | 1158 | CD1 | ILE | A | 490 | −5.849 | 11.484 | 29.192 | 1.00 | 25.52 | A | C |
| ATOM | 1162 | CG2 | ILE | A | 490 | −8.558 | 12.096 | 28.022 | 1.00 | 25.12 | A | C |
| ATOM | 1166 | C | ILE | A | 490 | −9.047 | 11.420 | 25.274 | 1.00 | 23.99 | A | C |
| ATOM | 1167 | O | ILE | A | 490 | −10.232 | 11.098 | 25.448 | 1.00 | 24.52 | A | O |
| ATOM | 1168 | N | GLY | A | 491 | −8.685 | 12.413 | 24.468 | 1.00 | 23.22 | A | N |
| ATOM | 1170 | CA | GLY | A | 491 | −9.677 | 13.139 | 23.681 | 1.00 | 23.44 | A | C |
| ATOM | 1173 | C | GLY | A | 491 | −9.145 | 13.889 | 22.481 | 1.00 | 23.06 | A | C |
| ATOM | 1174 | O | GLY | A | 491 | −7.944 | 14.066 | 22.320 | 1.00 | 21.24 | A | O |
| ATOM | 1175 | N | ILE | A | 492 | −10.069 | 14.324 | 21.623 | 1.00 | 23.34 | A | N |
| ATOM | 1177 | CA | ILE | A | 492 | −9.747 | 15.175 | 20.490 | 1.00 | 23.94 | A | C |
| ATOM | 1179 | CB | ILE | A | 492 | −9.914 | 14.394 | 19.167 | 1.00 | 24.29 | A | C |
| ATOM | 1181 | CG1 | ILE | A | 492 | −9.044 | 13.128 | 19.175 | 1.00 | 24.89 | A | C |
| ATOM | 1184 | CD1 | ILE | A | 492 | −9.389 | 12.139 | 18.099 | 1.00 | 26.68 | A | C |
| ATOM | 1188 | CG2 | ILE | A | 492 | −9.539 | 15.252 | 17.986 | 1.00 | 24.59 | A | C |
| ATOM | 1192 | C | ILE | A | 492 | −10.675 | 16.401 | 20.528 | 1.00 | 24.76 | A | C |
| ATOM | 1193 | O | ILE | A | 492 | −11.891 | 16.254 | 20.639 | 1.00 | 23.89 | A | O |
| ATOM | 1194 | N | ILE | A | 493 | −10.086 | 17.598 | 20.508 | 1.00 | 25.52 | A | N |
| ATOM | 1196 | CA | ILE | A | 493 | −10.828 | 18.833 | 20.257 | 1.00 | 26.54 | A | C |
| ATOM | 1198 | CB | ILE | A | 493 | −10.315 | 20.009 | 21.138 | 1.00 | 26.60 | A | C |
| ATOM | 1200 | CG1 | ILE | A | 493 | −10.215 | 19.614 | 22.613 | 1.00 | 26.82 | A | C |
| ATOM | 1203 | CD1 | ILE | A | 493 | −11.446 | 18.990 | 23.180 | 1.00 | 27.58 | A | C |
| ATOM | 1207 | CG2 | ILE | A | 493 | −11.217 | 21.249 | 20.961 | 1.00 | 26.95 | A | C |
| ATOM | 1211 | C | ILE | A | 493 | −10.609 | 19.151 | 18.785 | 1.00 | 27.17 | A | C |
| ATOM | 1212 | O | ILE | A | 493 | −9.531 | 19.576 | 18.404 | 1.00 | 26.34 | A | O |
| ATOM | 1213 | N | GLU | A | 494 | −11.628 | 18.927 | 17.959 | 1.00 | 28.32 | A | N |
| ATOM | 1215 | CA | GLU | A | 494 | −11.502 | 19.096 | 16.510 | 1.00 | 29.45 | A | C |
| ATOM | 1217 | CB | GLU | A | 494 | −12.698 | 18.444 | 15.800 | 1.00 | 30.09 | A | C |
| ATOM | 1220 | CG | GLU | A | 494 | −12.800 | 16.943 | 16.024 | 1.00 | 32.11 | A | C |
| ATOM | 1223 | CD | GLU | A | 494 | −13.958 | 16.292 | 15.280 | 1.00 | 35.78 | A | C |
| ATOM | 1224 | OE1 | GLU | A | 494 | −14.108 | 16.531 | 14.058 | 1.00 | 38.41 | A | O |
| ATOM | 1225 | OE2 | GLU | A | 494 | −14.717 | 15.524 | 15.918 | 1.00 | 38.33 | A | O |
| ATOM | 1226 | C | GLU | A | 494 | −11.390 | 20.567 | 16.098 | 1.00 | 29.72 | A | C |
| ATOM | 1227 | O | GLU | A | 494 | −10.667 | 20.900 | 15.160 | 1.00 | 29.12 | A | O |
| ATOM | 1228 | N | GLU | A | 495 | −12.073 | 21.432 | 16.846 | 1.00 | 30.47 | A | N |
| ATOM | 1230 | CA | GLU | A | 495 | −12.259 | 22.853 | 16.505 | 1.00 | 31.12 | A | C |
| ATOM | 1232 | CB | GLU | A | 495 | −13.324 | 23.465 | 17.435 | 1.00 | 31.66 | A | C |
| ATOM | 1235 | CG | GLU | A | 495 | −14.759 | 23.024 | 17.203 | 1.00 | 34.10 | A | C |
| ATOM | 1238 | CD | GLU | A | 495 | −14.995 | 21.541 | 17.444 | 1.00 | 36.85 | A | C |
| ATOM | 1239 | OE1 | GLU | A | 495 | −14.650 | 21.029 | 18.544 | 1.00 | 38.24 | A | O |
| ATOM | 1240 | OE2 | GLU | A | 495 | −15.521 | 20.885 | 16.517 | 1.00 | 39.02 | A | O |
| ATOM | 1241 | C | GLU | A | 495 | −10.978 | 23.695 | 16.633 | 1.00 | 30.92 | A | C |
| ATOM | 1242 | O | GLU | A | 495 | −9.950 | 23.203 | 17.113 | 1.00 | 30.08 | A | O |
| ATOM | 1243 | N | GLU | A | 496 | −11.084 | 24.966 | 16.207 | 1.00 | 30.74 | A | N |
| ATOM | 1245 | CA | GLU | A | 496 | −10.102 | 26.053 | 16.450 | 1.00 | 30.21 | A | C |
| ATOM | 1247 | CB | GLU | A | 496 | −10.331 | 26.722 | 17.809 | 1.00 | 30.71 | A | C |
| ATOM | 1250 | CG | GLU | A | 496 | −11.767 | 27.153 | 18.080 | 1.00 | 33.56 | A | C |
| ATOM | 1253 | CD | GLU | A | 496 | −12.218 | 28.297 | 17.176 | 1.00 | 37.40 | A | C |
| ATOM | 1254 | OE1 | GLU | A | 496 | −11.734 | 29.442 | 17.368 | 1.00 | 39.71 | A | O |
| ATOM | 1255 | OE2 | GLU | A | 496 | −13.057 | 28.053 | 16.273 | 1.00 | 39.53 | A | O |
| ATOM | 1256 | C | GLU | A | 496 | −8.688 | 25.536 | 16.214 | 1.00 | 28.69 | A | C |
| ATOM | 1257 | O | GLU | A | 496 | −8.518 | 24.890 | 15.181 | 1.00 | 29.82 | A | O |
| ATOM | 1258 | N | PRO | A | 497 | −7.673 | 25.765 | 17.068 | 1.00 | 26.40 | A | N |
| ATOM | 1259 | CA | PRO | A | 497 | −6.463 | 24.955 | 16.914 | 1.00 | 24.97 | A | C |
| ATOM | 1261 | CB | PRO | A | 497 | −5.435 | 25.650 | 17.812 | 1.00 | 25.01 | A | C |
| ATOM | 1264 | CG | PRO | A | 497 | −6.217 | 26.352 | 18.819 | 1.00 | 25.48 | A | C |
| ATOM | 1267 | CD | PRO | A | 497 | −7.508 | 26.726 | 18.176 | 1.00 | 26.20 | A | C |
| ATOM | 1270 | C | PRO | A | 497 | −6.785 | 23.545 | 17.400 | 1.00 | 23.59 | A | C |
| ATOM | 1271 | O | PRO | A | 497 | −7.238 | 23.365 | 18.525 | 1.00 | 21.60 | A | O |
| ATOM | 1272 | N | THR | A | 498 | −6.593 | 22.562 | 16.533 | 1.00 | 22.37 | A | N |
| ATOM | 1274 | CA | THR | A | 498 | −6.969 | 21.193 | 16.855 | 1.00 | 22.15 | A | C |
| ATOM | 1276 | CB | THR | A | 498 | −6.918 | 20.363 | 15.580 | 1.00 | 22.32 | A | C |
| ATOM | 1278 | OG1 | THR | A | 498 | −7.958 | 20.826 | 14.700 | 1.00 | 25.57 | A | O |
| ATOM | 1280 | CG2 | THR | A | 498 | −7.252 | 18.924 | 15.840 | 1.00 | 22.92 | A | C |
| ATOM | 1284 | C | THR | A | 498 | −6.022 | 20.654 | 17.922 | 1.00 | 20.09 | A | C |
| ATOM | 1285 | O | THR | A | 498 | −4.835 | 20.874 | 17.821 | 1.00 | 19.92 | A | O |
| ATOM | 1286 | N | TRP | A | 499 | −6.568 | 20.002 | 18.950 | 1.00 | 19.00 | A | N |
| ATOM | 1288 | CA | TRP | A | 499 | −5.777 | 19.413 | 20.040 | 1.00 | 17.23 | A | C |
| ATOM | 1290 | CB | TRP | A | 499 | −6.188 | 20.010 | 21.393 | 1.00 | 17.26 | A | C |
| ATOM | 1293 | CG | TRP | A | 499 | −5.734 | 21.440 | 21.693 | 1.00 | 16.11 | A | C |
| ATOM | 1294 | CD1 | TRP | A | 499 | −5.098 | 22.301 | 20.851 | 1.00 | 16.58 | A | C |
| ATOM | 1296 | NE1 | TRP | A | 499 | −4.865 | 23.499 | 21.483 | 1.00 | 15.91 | A | N |
| ATOM | 1298 | CE2 | TRP | A | 499 | −5.342 | 23.431 | 22.760 | 1.00 | 13.91 | A | C |
| ATOM | 1299 | CD2 | TRP | A | 499 | −5.906 | 22.149 | 22.927 | 1.00 | 14.32 | A | C |
| ATOM | 1300 | CE3 | TRP | A | 499 | −6.474 | 21.825 | 24.167 | 1.00 | 14.41 | A | C |
| ATOM | 1302 | CZ3 | TRP | A | 499 | −6.466 | 22.789 | 25.187 | 1.00 | 15.48 | A | C |
| ATOM | 1304 | CH2 | TRP | A | 499 | −5.914 | 24.063 | 24.973 | 1.00 | 15.79 | A | C |
| ATOM | 1306 | CZ2 | TRP | A | 499 | −5.345 | 24.401 | 23.770 | 1.00 | 16.05 | A | C |
| ATOM | 1308 | C | TRP | A | 499 | −6.011 | 17.891 | 20.113 | 1.00 | 16.56 | A | C |
| ATOM | 1309 | O | TRP | A | 499 | −7.161 | 17.444 | 20.219 | 1.00 | 16.56 | A | O |
| ATOM | 1310 | N | ILE | A | 500 | −4.933 | 17.111 | 20.082 | 1.00 | 15.45 | A | N |
| ATOM | 1312 | CA | ILE | A | 500 | −4.987 | 15.696 | 20.427 | 1.00 | 15.58 | A | C |
| ATOM | 1314 | CB | ILE | A | 500 | −4.126 | 14.870 | 19.461 | 1.00 | 16.46 | A | C |
| ATOM | 1316 | CG1 | ILE | A | 500 | −4.423 | 15.233 | 17.994 | 1.00 | 17.06 | A | C |
| ATOM | 1319 | CD1 | ILE | A | 500 | −5.887 | 15.183 | 17.626 | 1.00 | 18.75 | A | C |
| ATOM | 1323 | CG2 | ILE | A | 500 | −4.288 | 13.390 | 19.744 | 1.00 | 18.35 | A | C |
| ATOM | 1327 | C | ILE | A | 500 | −4.467 | 15.555 | 21.863 | 1.00 | 14.87 | A | C |
| ATOM | 1328 | O | ILE | A | 500 | −3.323 | 15.879 | 22.141 | 1.00 | 14.70 | A | O |
| ATOM | 1329 | N | ILE | A | 501 | −5.318 | 15.096 | 22.765 | 1.00 | 13.73 | A | N |
| ATOM | 1331 | CA | ILE | A | 501 | −4.998 | 14.985 | 24.178 | 1.00 | 13.36 | A | C |
| ATOM | 1333 | CB | ILE | A | 501 | −6.190 | 15.451 | 25.034 | 1.00 | 12.71 | A | C |
| ATOM | 1335 | CG1 | ILE | A | 501 | −6.719 | 16.788 | 24.496 | 1.00 | 14.26 | A | C |
| ATOM | 1338 | CD1 | ILE | A | 501 | −7.978 | 17.256 | 25.129 | 1.00 | 15.07 | A | C |
| ATOM | 1342 | CG2 | ILE | A | 501 | −5.768 | 15.583 | 26.483 | 1.00 | 12.38 | A | C |
| ATOM | 1346 | C | ILE | A | 501 | −4.629 | 13.546 | 24.539 | 1.00 | 13.36 | A | C |
| ATOM | 1347 | O | ILE | A | 501 | −5.469 | 12.638 | 24.448 | 1.00 | 13.51 | A | O |
| ATOM | 1348 | N | MET | A | 502 | −3.372 | 13.359 | 24.933 | 1.00 | 13.48 | A | N |
| ATOM | 1350 | CA | MET | A | 502 | −2.836 | 12.061 | 25.353 | 1.00 | 13.96 | A | C |
| ATOM | 1352 | CB | MET | A | 502 | −1.550 | 11.754 | 24.585 | 1.00 | 14.71 | A | C |
| ATOM | 1355 | CG | MET | A | 502 | −1.634 | 11.957 | 23.074 | 1.00 | 18.44 | A | C |
| ATOM | 1358 | SD | MET | A | 502 | −2.691 | 10.736 | 22.262 | 1.00 | 25.96 | A | S |
| ATOM | 1359 | CE | MET | A | 502 | −1.705 | 9.251 | 22.464 | 1.00 | 24.95 | A | C |
| ATOM | 1363 | C | MET | A | 502 | −2.498 | 12.057 | 26.845 | 1.00 | 13.14 | A | C |
| ATOM | 1364 | O | MET | A | 502 | −2.200 | 13.092 | 27.436 | 1.00 | 11.75 | A | O |
| ATOM | 1365 | N | GLU | A | 503 | −2.501 | 10.879 | 27.458 | 1.00 | 12.97 | A | N |
| ATOM | 1367 | CA | GLU | A | 503 | −1.947 | 10.721 | 28.784 | 1.00 | 13.82 | A | C |
| ATOM | 1369 | CB | GLU | A | 503 | −2.136 | 9.258 | 29.215 | 1.00 | 15.08 | A | C |
| ATOM | 1372 | CG | GLU | A | 503 | −1.603 | 8.887 | 30.583 | 1.00 | 18.56 | A | C |
| ATOM | 1375 | CD | GLU | A | 503 | −2.097 | 7.516 | 31.024 | 1.00 | 23.89 | A | C |
| ATOM | 1376 | OE1 | GLU | A | 503 | −2.315 | 6.636 | 30.153 | 1.00 | 26.84 | A | O |
| ATOM | 1377 | OE2 | GLU | A | 503 | −2.272 | 7.314 | 32.243 | 1.00 | 29.32 | A | O |
| ATOM | 1378 | C | GLU | A | 503 | −0.457 | 11.112 | 28.747 | 1.00 | 13.19 | A | C |
| ATOM | 1379 | O | GLU | A | 503 | 0.230 | 10.778 | 27.787 | 1.00 | 13.15 | A | O |
| ATOM | 1380 | N | LEU | A | 504 | 0.020 | 11.836 | 29.760 | 1.00 | 12.59 | A | N |
| ATOM | 1382 | CA | LEU | A | 504 | 1.440 | 12.210 | 29.862 | 1.00 | 13.43 | A | C |
| ATOM | 1384 | CB | LEU | A | 504 | 1.635 | 13.449 | 30.746 | 1.00 | 13.57 | A | C |
| ATOM | 1387 | CG | LEU | A | 504 | 3.065 | 13.981 | 30.844 | 1.00 | 14.83 | A | C |
| ATOM | 1389 | CD1 | LEU | A | 504 | 3.440 | 14.719 | 29.551 | 1.00 | 15.70 | A | C |
| ATOM | 1393 | CD2 | LEU | A | 504 | 3.281 | 14.893 | 32.069 | 1.00 | 17.43 | A | C |
| ATOM | 1397 | C | LEU | A | 504 | 2.237 | 11.049 | 30.443 | 1.00 | 13.16 | A | C |
| ATOM | 1398 | O | LEU | A | 504 | 1.831 | 10.448 | 31.436 | 1.00 | 12.13 | A | O |
| ATOM | 1399 | N | TYR | A | 505 | 3.361 | 10.740 | 29.807 | 1.00 | 13.87 | A | N |
| ATOM | 1401 | CA | TYR | A | 505 | 4.261 | 9.682 | 30.247 | 1.00 | 14.37 | A | C |
| ATOM | 1403 | CB | TYR | A | 505 | 4.379 | 8.582 | 29.159 | 1.00 | 15.13 | A | C |
| ATOM | 1406 | CG | TYR | A | 505 | 3.034 | 7.948 | 28.815 | 1.00 | 14.57 | A | C |
| ATOM | 1407 | CD1 | TYR | A | 505 | 2.254 | 7.352 | 29.802 | 1.00 | 16.24 | A | C |
| ATOM | 1409 | CE1 | TYR | A | 505 | 0.996 | 6.804 | 29.506 | 1.00 | 15.83 | A | C |
| ATOM | 1411 | CZ | TYR | A | 505 | 0.517 | 6.853 | 28.221 | 1.00 | 13.11 | A | C |
| ATOM | 1412 | OH | TYR | A | 505 | −0.713 | 6.332 | 27.933 | 1.00 | 15.41 | A | O |
| ATOM | 1414 | CE2 | TYR | A | 505 | 1.255 | 7.455 | 27.229 | 1.00 | 13.32 | A | C |
| ATOM | 1416 | CD2 | TYR | A | 505 | 2.506 | 8.017 | 27.532 | 1.00 | 14.19 | A | C |
| ATOM | 1418 | C | TYR | A | 505 | 5.566 | 10.408 | 30.557 | 1.00 | 14.61 | A | C |
| ATOM | 1419 | O | TYR | A | 505 | 6.379 | 10.672 | 29.675 | 1.00 | 13.90 | A | O |
| ATOM | 1420 | N | PRO | A | 506 | 5.707 | 10.836 | 31.812 | 1.00 | 15.51 | A | N |
| ATOM | 1421 | CA | PRO | A | 506 | 6.699 | 11.840 | 32.188 | 1.00 | 15.79 | A | C |
| ATOM | 1423 | CB | PRO | A | 506 | 6.291 | 12.186 | 33.616 | 1.00 | 16.40 | A | C |
| ATOM | 1426 | CG | PRO | A | 506 | 5.692 | 10.962 | 34.115 | 1.00 | 16.38 | A | C |
| ATOM | 1429 | CD | PRO | A | 506 | 4.903 | 10.414 | 32.974 | 1.00 | 16.03 | A | C |
| ATOM | 1432 | C | PRO | A | 506 | 8.168 | 11.364 | 32.119 | 1.00 | 15.41 | A | C |
| ATOM | 1433 | O | PRO | A | 506 | 9.055 | 12.186 | 32.029 | 1.00 | 15.80 | A | O |
| ATOM | 1434 | N | TYR | A | 507 | 8.406 | 10.062 | 32.106 | 1.00 | 14.60 | A | N |
| ATOM | 1436 | CA | TYR | A | 507 | 9.767 | 9.548 | 31.946 | 1.00 | 13.73 | A | C |
| ATOM | 1438 | CB | TYR | A | 507 | 9.872 | 8.143 | 32.516 | 1.00 | 13.74 | A | C |
| ATOM | 1441 | CG | TYR | A | 507 | 9.659 | 8.071 | 33.997 | 1.00 | 14.65 | A | C |
| ATOM | 1442 | CD1 | TYR | A | 507 | 10.704 | 8.269 | 34.879 | 1.00 | 15.68 | A | C |
| ATOM | 1444 | CE1 | TYR | A | 507 | 10.505 | 8.191 | 36.268 | 1.00 | 17.32 | A | C |
| ATOM | 1446 | CZ | TYR | A | 507 | 9.241 | 7.931 | 36.754 | 1.00 | 16.75 | A | C |
| ATOM | 1447 | OH | TYR | A | 507 | 9.002 | 7.873 | 38.099 | 1.00 | 19.60 | A | O |
| ATOM | 1449 | CE2 | TYR | A | 507 | 8.198 | 7.737 | 35.895 | 1.00 | 15.81 | A | C |
| ATOM | 1451 | CD2 | TYR | A | 507 | 8.408 | 7.807 | 34.521 | 1.00 | 14.55 | A | C |
| ATOM | 1453 | C | TYR | A | 507 | 10.276 | 9.550 | 30.491 | 1.00 | 13.31 | A | C |
| ATOM | 1454 | O | TYR | A | 507 | 11.451 | 9.268 | 30.268 | 1.00 | 13.27 | A | O |
| ATOM | 1455 | N | GLY | A | 508 | 9.411 | 9.838 | 29.516 | 1.00 | 12.24 | A | N |
| ATOM | 1457 | CA | GLY | A | 508 | 9.834 | 10.058 | 28.142 | 1.00 | 12.21 | A | C |
| ATOM | 1460 | C | GLY | A | 508 | 10.112 | 8.784 | 27.362 | 1.00 | 11.49 | A | C |
| ATOM | 1461 | O | GLY | A | 508 | 9.628 | 7.729 | 27.727 | 1.00 | 10.44 | A | O |
| ATOM | 1462 | N | GLU | A | 509 | 10.885 | 8.896 | 26.281 | 1.00 | 11.21 | A | N |
| ATOM | 1464 | CA | GLU | A | 509 | 11.183 | 7.744 | 25.416 | 1.00 | 11.54 | A | C |
| ATOM | 1466 | CB | GLU | A | 509 | 11.913 | 8.194 | 24.156 | 1.00 | 12.66 | A | C |
| ATOM | 1469 | CG | GLU | A | 509 | 11.143 | 9.164 | 23.279 | 1.00 | 15.29 | A | C |
| ATOM | 1472 | CD | GLU | A | 509 | 11.973 | 9.695 | 22.119 | 1.00 | 18.15 | A | C |
| ATOM | 1473 | OE1 | GLU | A | 509 | 13.122 | 9.251 | 21.947 | 1.00 | 19.31 | A | O |
| ATOM | 1474 | OE2 | GLU | A | 509 | 11.472 | 10.588 | 21.393 | 1.00 | 21.32 | A | O |
| ATOM | 1475 | C | GLU | A | 509 | 12.038 | 6.666 | 26.095 | 1.00 | 10.37 | A | C |
| ATOM | 1476 | O | GLU | A | 509 | 12.953 | 6.974 | 26.847 | 1.00 | 10.56 | A | O |
| ATOM | 1477 | N | LEU | A | 510 | 11.742 | 5.399 | 25.791 | 1.00 | 9.52 | A | N |
| ATOM | 1479 | CA | LEU | A | 510 | 12.447 | 4.275 | 26.392 | 1.00 | 9.25 | A | C |
| ATOM | 1481 | CB | LEU | A | 510 | 11.768 | 2.956 | 25.990 | 1.00 | 9.16 | A | C |
| ATOM | 1484 | CG | LEU | A | 510 | 12.395 | 1.682 | 26.566 | 1.00 | 8.82 | A | C |
| ATOM | 1486 | CD1 | LEU | A | 510 | 12.453 | 1.737 | 28.081 | 1.00 | 9.86 | A | C |
| ATOM | 1490 | CD2 | LEU | A | 510 | 11.596 | 0.465 | 26.104 | 1.00 | 9.45 | A | C |
| ATOM | 1494 | C | LEU | A | 510 | 13.948 | 4.249 | 26.057 | 1.00 | 9.51 | A | C |
| ATOM | 1495 | O | LEU | A | 510 | 14.772 | 3.937 | 26.925 | 1.00 | 9.34 | A | O |
| ATOM | 1496 | N | GLY | A | 511 | 14.322 | 4.610 | 24.824 | 1.00 | 9.82 | A | N |
| ATOM | 1498 | CA | GLY | A | 511 | 15.729 | 4.549 | 24.433 | 1.00 | 10.57 | A | C |
| ATOM | 1501 | C | GLY | A | 511 | 16.585 | 5.425 | 25.340 | 1.00 | 10.99 | A | C |
| ATOM | 1502 | O | GLY | A | 511 | 17.566 | 4.964 | 25.934 | 1.00 | 11.11 | A | O |
| ATOM | 1503 | N | HIS | A | 512 | 16.178 | 6.672 | 25.492 | 1.00 | 11.55 | A | N |
| ATOM | 1505 | CA | HIS | A | 512 | 16.875 | 7.608 | 26.374 | 1.00 | 12.69 | A | C |
| ATOM | 1507 | CB | HIS | A | 512 | 16.319 | 9.011 | 26.174 | 1.00 | 13.88 | A | C |
| ATOM | 1510 | CG | HIS | A | 512 | 16.559 | 9.541 | 24.795 | 1.00 | 17.28 | A | C |
| ATOM | 1511 | ND1 | HIS | A | 512 | 17.678 | 9.214 | 24.061 | 1.00 | 22.03 | A | N |
| ATOM | 1513 | CE1 | HIS | A | 512 | 17.618 | 9.808 | 22.882 | 1.00 | 22.98 | A | C |
| ATOM | 1515 | NE2 | HIS | A | 512 | 16.508 | 10.518 | 22.831 | 1.00 | 21.48 | A | N |
| ATOM | 1517 | CD2 | HIS | A | 512 | 15.821 | 10.360 | 24.011 | 1.00 | 20.94 | A | C |
| ATOM | 1519 | C | HIS | A | 512 | 16.810 | 7.196 | 27.840 | 1.00 | 11.73 | A | C |
| ATOM | 1520 | O | HIS | A | 512 | 17.795 | 7.308 | 28.560 | 1.00 | 11.30 | A | O |
| ATOM | 1521 | N | TYR | A | 513 | 15.664 | 6.682 | 28.269 | 1.00 | 10.95 | A | N |
| ATOM | 1523 | CA | TYR | A | 513 | 15.472 | 6.184 | 29.630 | 1.00 | 10.24 | A | C |
| ATOM | 1525 | CB | TYR | A | 513 | 14.034 | 5.677 | 29.797 | 1.00 | 10.11 | A | C |
| ATOM | 1528 | CG | TYR | A | 513 | 13.693 | 5.094 | 31.169 | 1.00 | 9.46 | A | C |
| ATOM | 1529 | CD1 | TYR | A | 513 | 13.157 | 5.901 | 32.160 | 1.00 | 9.43 | A | C |
| ATOM | 1531 | CE1 | TYR | A | 513 | 12.800 | 5.391 | 33.393 | 1.00 | 9.59 | A | C |
| ATOM | 1533 | CZ | TYR | A | 513 | 12.988 | 4.068 | 33.671 | 1.00 | 9.14 | A | C |
| ATOM | 1534 | OH | TYR | A | 513 | 12.628 | 3.617 | 34.912 | 1.00 | 12.42 | A | O |
| ATOM | 1536 | CE2 | TYR | A | 513 | 13.533 | 3.214 | 32.708 | 1.00 | 8.34 | A | C |
| ATOM | 1538 | CD2 | TYR | A | 513 | 13.854 | 3.735 | 31.452 | 1.00 | 8.09 | A | C |
| ATOM | 1540 | C | TYR | A | 513 | 16.469 | 5.068 | 29.965 | 1.00 | 10.47 | A | C |
| ATOM | 1541 | O | TYR | A | 513 | 17.110 | 5.099 | 31.009 | 1.00 | 10.13 | A | O |
| ATOM | 1542 | N | LEU | A | 514 | 16.623 | 4.102 | 29.058 | 1.00 | 10.69 | A | N |
| ATOM | 1544 | CA | LEU | A | 514 | 17.596 | 3.021 | 29.239 | 1.00 | 11.38 | A | C |
| ATOM | 1546 | CB | LEU | A | 514 | 17.478 | 1.997 | 28.115 | 1.00 | 11.46 | A | C |
| ATOM | 1549 | CG | LEU | A | 514 | 16.163 | 1.198 | 28.035 | 1.00 | 12.98 | A | C |
| ATOM | 1551 | CD1 | LEU | A | 514 | 16.082 | 0.442 | 26.729 | 1.00 | 14.53 | A | C |
| ATOM | 1555 | CD2 | LEU | A | 514 | 16.010 | 0.260 | 29.218 | 1.00 | 12.92 | A | C |
| ATOM | 1559 | C | LEU | A | 514 | 19.027 | 3.556 | 29.300 | 1.00 | 12.00 | A | C |
| ATOM | 1560 | O | LEU | A | 514 | 19.839 | 3.080 | 30.090 | 1.00 | 12.23 | A | O |
| ATOM | 1561 | N | GLU | A | 515 | 19.330 | 4.518 | 28.450 | 1.00 | 12.66 | A | N |
| ATOM | 1563 | CA | GLU | A | 515 | 20.650 | 5.160 | 28.447 | 1.00 | 14.53 | A | C |
| ATOM | 1565 | CB | GLU | A | 515 | 20.742 | 6.212 | 27.346 | 1.00 | 15.14 | A | C |
| ATOM | 1568 | CG | GLU | A | 515 | 20.701 | 5.656 | 25.929 | 1.00 | 19.10 | A | C |
| ATOM | 1571 | CD | GLU | A | 515 | 20.531 | 6.744 | 24.868 | 1.00 | 24.40 | A | C |
| ATOM | 1572 | OE1 | GLU | A | 515 | 20.496 | 7.948 | 25.236 | 1.00 | 29.66 | A | O |
| ATOM | 1573 | OE2 | GLU | A | 515 | 20.433 | 6.395 | 23.665 | 1.00 | 28.24 | A | O |
| ATOM | 1574 | C | GLU | A | 515 | 20.954 | 5.812 | 29.795 | 1.00 | 15.17 | A | C |
| ATOM | 1575 | O | GLU | A | 515 | 22.046 | 5.601 | 30.347 | 1.00 | 15.52 | A | O |
| ATOM | 1576 | N | ARG | A | 516 | 19.987 | 6.576 | 30.317 | 1.00 | 15.35 | A | N |
| ATOM | 1578 | CA | ARG | A | 516 | 20.114 | 7.306 | 31.595 | 1.00 | 16.48 | A | C |
| ATOM | 1580 | CB | ARG | A | 516 | 18.842 | 8.134 | 31.882 | 1.00 | 17.00 | A | C |
| ATOM | 1583 | CG | ARG | A | 516 | 18.722 | 9.407 | 31.115 | 1.00 | 19.06 | A | C |
| ATOM | 1586 | CD | ARG | A | 516 | 17.815 | 10.406 | 31.760 | 1.00 | 18.75 | A | C |
| ATOM | 1589 | NE | ARG | A | 516 | 16.482 | 9.894 | 32.079 | 1.00 | 17.60 | A | N |
| ATOM | 1591 | CZ | ARG | A | 516 | 15.500 | 9.702 | 31.197 | 1.00 | 17.67 | A | C |
| ATOM | 1592 | NH1 | ARG | A | 516 | 15.669 | 9.969 | 29.909 | 1.00 | 17.37 | A | N |
| ATOM | 1595 | NH2 | ARG | A | 516 | 14.323 | 9.248 | 31.612 | 1.00 | 20.07 | A | N |
| ATOM | 1598 | C | ARG | A | 516 | 20.298 | 6.382 | 32.790 | 1.00 | 16.29 | A | C |
| ATOM | 1599 | O | ARG | A | 516 | 21.032 | 6.702 | 33.743 | 1.00 | 15.28 | A | O |
| ATOM | 1600 | N | ASN | A | 517 | 19.609 | 5.245 | 32.745 | 1.00 | 16.03 | A | N |
| ATOM | 1602 | CA | ASN | A | 517 | 19.387 | 4.432 | 33.927 | 1.00 | 15.86 | A | C |
| ATOM | 1604 | CB | ASN | A | 517 | 17.876 | 4.249 | 34.155 | 1.00 | 15.83 | A | C |
| ATOM | 1607 | CG | ASN | A | 517 | 17.161 | 5.562 | 34.430 | 1.00 | 16.01 | A | C |
| ATOM | 1608 | OD1 | ASN | A | 517 | 16.182 | 5.918 | 33.758 | 1.00 | 16.60 | A | O |
| ATOM | 1609 | ND2 | ASN | A | 517 | 17.671 | 6.316 | 35.390 | 1.00 | 15.03 | A | N |
| ATOM | 1612 | C | ASN | A | 517 | 20.108 | 3.092 | 33.863 | 1.00 | 16.06 | A | C |
| ATOM | 1613 | O | ASN | A | 517 | 19.900 | 2.238 | 34.715 | 1.00 | 15.77 | A | O |
| ATOM | 1614 | N | LYS | A | 518 | 20.980 | 2.928 | 32.870 | 1.00 | 16.16 | A | N |
| ATOM | 1616 | CA | LYS | A | 518 | 21.613 | 1.641 | 32.606 | 1.00 | 17.63 | A | C |
| ATOM | 1618 | CB | LYS | A | 518 | 22.643 | 1.784 | 31.474 | 1.00 | 17.77 | A | C |
| ATOM | 1621 | CG | LYS | A | 518 | 23.632 | 0.636 | 31.368 | 1.00 | 20.82 | A | C |
| ATOM | 1624 | CD | LYS | A | 518 | 24.421 | 0.686 | 30.063 | 1.00 | 24.15 | A | C |
| ATOM | 1627 | CE | LYS | A | 518 | 25.172 | 1.979 | 29.909 | 1.00 | 26.03 | A | C |
| ATOM | 1630 | NZ | LYS | A | 518 | 24.304 | 3.106 | 29.435 | 1.00 | 28.18 | A | N |
| ATOM | 1634 | C | LYS | A | 518 | 22.254 | 1.017 | 33.849 | 1.00 | 18.11 | A | C |
| ATOM | 1635 | O | LYS | A | 518 | 22.126 | −0.189 | 34.086 | 1.00 | 18.39 | A | O |
| ATOM | 1636 | N | ASN | A | 519 | 22.938 | 1.825 | 34.652 | 1.00 | 18.49 | A | N |
| ATOM | 1638 | CA | ASN | A | 519 | 23.671 | 1.287 | 35.802 | 1.00 | 19.30 | A | C |
| ATOM | 1640 | CB | ASN | A | 519 | 24.611 | 2.346 | 36.375 | 1.00 | 19.86 | A | C |
| ATOM | 1643 | CG | ASN | A | 519 | 25.664 | 2.759 | 35.382 | 1.00 | 21.38 | A | C |
| ATOM | 1644 | OD1 | ASN | A | 519 | 26.149 | 1.930 | 34.612 | 1.00 | 26.70 | A | O |
| ATOM | 1645 | ND2 | ASN | A | 519 | 26.012 | 4.042 | 35.372 | 1.00 | 28.25 | A | N |
| ATOM | 1648 | C | ASN | A | 519 | 22.823 | 0.691 | 36.922 | 1.00 | 19.78 | A | C |
| ATOM | 1649 | O | ASN | A | 519 | 23.344 | −0.079 | 37.710 | 1.00 | 19.38 | A | O |
| ATOM | 1650 | N | SER | A | 520 | 21.531 | 1.029 | 36.985 | 1.00 | 19.92 | A | N |
| ATOM | 1652 | CA | SER | A | 520 | 20.647 | 0.503 | 38.031 | 1.00 | 20.71 | A | C |
| ATOM | 1654 | CB | SER | A | 520 | 20.044 | 1.663 | 38.834 | 1.00 | 21.31 | A | C |
| ATOM | 1657 | OG | SER | A | 520 | 19.120 | 2.400 | 38.069 | 1.00 | 23.80 | A | O |
| ATOM | 1659 | C | SER | A | 520 | 19.536 | −0.435 | 37.536 | 1.00 | 20.17 | A | C |
| ATOM | 1660 | O | SER | A | 520 | 18.775 | −0.953 | 38.335 | 1.00 | 21.79 | A | O |
| ATOM | 1661 | N | LEU | A | 521 | 19.456 | −0.678 | 36.234 | 1.00 | 18.62 | A | N |
| ATOM | 1663 | CA | LEU | A | 521 | 18.417 | −1.545 | 35.676 | 1.00 | 17.54 | A | C |
| ATOM | 1665 | CB | LEU | A | 521 | 18.209 | −1.218 | 34.192 | 1.00 | 17.30 | A | C |
| ATOM | 1668 | CG | LEU | A | 521 | 17.417 | 0.046 | 33.909 | 1.00 | 17.34 | A | C |
| ATOM | 1670 | CD1 | LEU | A | 521 | 17.651 | 0.555 | 32.493 | 1.00 | 17.37 | A | C |
| ATOM | 1674 | CD2 | LEU | A | 521 | 15.950 | −0.230 | 34.136 | 1.00 | 19.13 | A | C |
| ATOM | 1678 | C | LEU | A | 521 | 18.794 | −3.014 | 35.804 | 1.00 | 16.93 | A | C |
| ATOM | 1679 | O | LEU | A | 521 | 19.938 | −3.378 | 35.566 | 1.00 | 17.89 | A | O |
| ATOM | 1680 | N | LYS | A | 522 | 17.841 | −3.863 | 36.164 | 1.00 | 15.75 | A | N |
| ATOM | 1682 | CA | LYS | A | 522 | 18.070 | −5.314 | 36.231 | 1.00 | 15.93 | A | C |
| ATOM | 1684 | CB | LYS | A | 522 | 17.277 | −5.944 | 37.385 | 1.00 | 16.21 | A | C |
| ATOM | 1687 | CG | LYS | A | 522 | 17.444 | −5.237 | 38.719 | 1.00 | 18.79 | A | C |
| ATOM | 1690 | CD | LYS | A | 522 | 16.528 | −5.804 | 39.802 | 1.00 | 21.17 | A | C |
| ATOM | 1693 | CE | LYS | A | 522 | 15.090 | −5.353 | 39.648 | 1.00 | 23.90 | A | C |
| ATOM | 1696 | NZ | LYS | A | 522 | 14.842 | −3.888 | 39.964 | 1.00 | 25.89 | A | N |
| ATOM | 1700 | C | LYS | A | 522 | 17.623 | −5.956 | 34.915 | 1.00 | 14.75 | A | C |
| ATOM | 1701 | O | LYS | A | 522 | 16.727 | −5.432 | 34.257 | 1.00 | 14.52 | A | O |
| ATOM | 1702 | N | VAL | A | 523 | 18.223 | −7.096 | 34.556 | 1.00 | 13.60 | A | N |
| ATOM | 1704 | CA | VAL | A | 523 | 17.821 | −7.845 | 33.349 | 1.00 | 13.60 | A | C |
| ATOM | 1706 | CB | VAL | A | 523 | 18.666 | −9.113 | 33.137 | 1.00 | 13.75 | A | C |
| ATOM | 1708 | CG1 | VAL | A | 523 | 18.182 | −9.891 | 31.939 | 1.00 | 13.84 | A | C |
| ATOM | 1712 | CG2 | VAL | A | 523 | 20.157 | −8.741 | 32.968 | 1.00 | 14.55 | A | C |
| ATOM | 1716 | C | VAL | A | 523 | 16.344 | −8.238 | 33.420 | 1.00 | 13.39 | A | C |
| ATOM | 1717 | O | VAL | A | 523 | 15.631 | −8.222 | 32.427 | 1.00 | 12.33 | A | O |
| ATOM | 1718 | N | LEU | A | 524 | 15.887 | −8.535 | 34.626 | 1.00 | 13.26 | A | N |
| ATOM | 1720 | CA | LEU | A | 524 | 14.512 | −8.886 | 34.875 | 1.00 | 14.13 | A | C |
| ATOM | 1722 | CB | LEU | A | 524 | 14.392 | −9.107 | 36.382 | 1.00 | 15.52 | A | C |
| ATOM | 1725 | CG | LEU | A | 524 | 13.090 | −9.468 | 37.032 | 1.00 | 20.72 | A | C |
| ATOM | 1727 | CD1 | LEU | A | 524 | 12.523 | −10.698 | 36.358 | 1.00 | 22.55 | A | C |
| ATOM | 1731 | CD2 | LEU | A | 524 | 13.407 | −9.706 | 38.518 | 1.00 | 22.42 | A | C |
| ATOM | 1735 | C | LEU | A | 524 | 13.556 | −7.804 | 34.362 | 1.00 | 12.61 | A | C |
| ATOM | 1736 | O | LEU | A | 524 | 12.528 | −8.109 | 33.738 | 1.00 | 11.66 | A | O |
| ATOM | 1737 | N | THR | A | 525 | 13.909 | −6.544 | 34.590 | 1.00 | 11.21 | A | N |
| ATOM | 1739 | CA | THR | A | 525 | 13.113 | −5.402 | 34.123 | 1.00 | 11.25 | A | C |
| ATOM | 1741 | CB | THR | A | 525 | 13.650 | −4.120 | 34.788 | 1.00 | 11.51 | A | C |
| ATOM | 1743 | OG1 | THR | A | 525 | 13.552 | −4.260 | 36.215 | 1.00 | 13.76 | A | O |
| ATOM | 1745 | CG2 | THR | A | 525 | 12.815 | −2.922 | 34.467 | 1.00 | 11.96 | A | C |
| ATOM | 1749 | C | THR | A | 525 | 13.124 | −5.235 | 32.600 | 1.00 | 10.93 | A | C |
| ATOM | 1750 | O | THR | A | 525 | 12.114 | −4.850 | 32.001 | 1.00 | 10.34 | A | O |
| ATOM | 1751 | N | LEU | A | 526 | 14.275 | −5.482 | 31.987 | 1.00 | 9.76 | A | N |
| ATOM | 1753 | CA | LEU | A | 526 | 14.412 | −5.405 | 30.523 | 1.00 | 9.91 | A | C |
| ATOM | 1755 | CB | LEU | A | 526 | 15.877 | −5.597 | 30.105 | 1.00 | 9.71 | A | C |
| ATOM | 1758 | CG | LEU | A | 526 | 16.878 | −4.621 | 30.717 | 1.00 | 10.00 | A | C |
| ATOM | 1760 | CD1 | LEU | A | 526 | 18.268 | −4.904 | 30.219 | 1.00 | 10.63 | A | C |
| ATOM | 1764 | CD2 | LEU | A | 526 | 16.484 | −3.208 | 30.422 | 1.00 | 10.98 | A | C |
| ATOM | 1768 | C | LEU | A | 526 | 13.520 | −6.455 | 29.836 | 1.00 | 9.82 | A | C |
| ATOM | 1769 | O | LEU | A | 526 | 12.876 | −6.176 | 28.822 | 1.00 | 8.11 | A | O |
| ATOM | 1770 | N | VAL | A | 527 | 13.475 | −7.660 | 30.411 | 1.00 | 9.84 | A | N |
| ATOM | 1772 | CA | VAL | A | 527 | 12.588 | −8.709 | 29.924 | 1.00 | 10.01 | A | C |
| ATOM | 1774 | CB | VAL | A | 527 | 12.910 | −10.075 | 30.588 | 1.00 | 10.28 | A | C |
| ATOM | 1776 | CG1 | VAL | A | 527 | 11.950 | −11.157 | 30.103 | 1.00 | 12.12 | A | C |
| ATOM | 1780 | CG2 | VAL | A | 527 | 14.326 | −10.464 | 30.275 | 1.00 | 11.61 | A | C |
| ATOM | 1784 | C | VAL | A | 527 | 11.111 | −8.316 | 30.132 | 1.00 | 9.91 | A | C |
| ATOM | 1785 | O | VAL | A | 527 | 10.286 | −8.533 | 29.247 | 1.00 | 9.20 | A | O |
| ATOM | 1786 | N | LEU | A | 528 | 10.783 | −7.715 | 31.277 | 1.00 | 9.17 | A | N |
| ATOM | 1788 | CA | LEU | A | 528 | 9.419 | −7.239 | 31.548 | 1.00 | 9.55 | A | C |
| ATOM | 1790 | CB | LEU | A | 528 | 9.308 | −6.582 | 32.940 | 1.00 | 10.47 | A | C |
| ATOM | 1793 | CG | LEU | A | 528 | 7.962 | −5.908 | 33.230 | 1.00 | 11.24 | A | C |
| ATOM | 1795 | CD1 | LEU | A | 528 | 6.855 | −6.956 | 33.158 | 1.00 | 11.66 | A | C |
| ATOM | 1799 | CD2 | LEU | A | 528 | 7.961 | −5.179 | 34.544 | 1.00 | 12.72 | A | C |
| ATOM | 1803 | C | LEU | A | 528 | 8.976 | −6.263 | 30.464 | 1.00 | 9.67 | A | C |
| ATOM | 1804 | O | LEU | A | 528 | 7.878 | −6.389 | 29.928 | 1.00 | 8.64 | A | O |
| ATOM | 1805 | N | TYR | A | 529 | 9.825 | −5.290 | 30.120 | 1.00 | 9.08 | A | N |
| ATOM | 1807 | CA | TYR | A | 529 | 9.447 | −4.301 | 29.091 | 1.00 | 8.78 | A | C |
| ATOM | 1809 | CB | TYR | A | 529 | 10.522 | −3.215 | 28.939 | 1.00 | 8.57 | A | C |
| ATOM | 1812 | CG | TYR | A | 529 | 10.744 | −2.321 | 30.143 | 1.00 | 10.12 | A | C |
| ATOM | 1813 | CD1 | TYR | A | 529 | 9.833 | −2.257 | 31.193 | 1.00 | 10.85 | A | C |
| ATOM | 1815 | CE1 | TYR | A | 529 | 10.057 | −1.438 | 32.298 | 1.00 | 12.11 | A | C |
| ATOM | 1817 | CZ | TYR | A | 529 | 11.211 | −0.677 | 32.365 | 1.00 | 13.84 | A | C |
| ATOM | 1818 | OH | TYR | A | 529 | 11.441 | 0.140 | 33.449 | 1.00 | 16.48 | A | O |
| ATOM | 1820 | CE2 | TYR | A | 529 | 12.137 | −0.737 | 31.354 | 1.00 | 11.18 | A | C |
| ATOM | 1822 | CD2 | TYR | A | 529 | 11.903 | −1.565 | 30.248 | 1.00 | 11.83 | A | C |
| ATOM | 1824 | C | TYR | A | 529 | 9.202 | −4.967 | 27.734 | 1.00 | 8.22 | A | C |
| ATOM | 1825 | O | TYR | A | 529 | 8.255 | −4.615 | 27.028 | 1.00 | 9.04 | A | O |
| ATOM | 1826 | N | SER | A | 530 | 10.036 | −5.937 | 27.390 | 1.00 | 8.24 | A | N |
| ATOM | 1828 | CA | SER | A | 530 | 9.889 | −6.722 | 26.154 | 1.00 | 7.83 | A | C |
| ATOM | 1830 | CB | SER | A | 530 | 11.052 | −7.730 | 26.015 | 1.00 | 7.54 | A | C |
| ATOM | 1833 | OG | SER | A | 530 | 12.310 | −7.077 | 25.839 | 1.00 | 10.28 | A | O |
| ATOM | 1835 | C | SER | A | 530 | 8.543 | −7.458 | 26.137 | 1.00 | 7.48 | A | C |
| ATOM | 1836 | O | SER | A | 530 | 7.820 | −7.443 | 25.141 | 1.00 | 7.17 | A | O |
| ATOM | 1837 | N | LEU | A | 531 | 8.199 | −8.074 | 27.258 | 1.00 | 6.73 | A | N |
| ATOM | 1839 | CA | LEU | A | 531 | 6.931 | −8.800 | 27.389 | 1.00 | 7.75 | A | C |
| ATOM | 1841 | CB | LEU | A | 531 | 6.912 | −9.561 | 28.728 | 1.00 | 7.60 | A | C |
| ATOM | 1844 | CG | LEU | A | 531 | 5.618 | −10.232 | 29.178 | 1.00 | 8.41 | A | C |
| ATOM | 1846 | CD1 | LEU | A | 531 | 5.201 | −11.263 | 28.153 | 1.00 | 9.01 | A | C |
| ATOM | 1850 | CD2 | LEU | A | 531 | 5.776 | −10.881 | 30.561 | 1.00 | 11.57 | A | C |
| ATOM | 1854 | C | LEU | A | 531 | 5.731 | −7.876 | 27.280 | 1.00 | 7.08 | A | C |
| ATOM | 1855 | O | LEU | A | 531 | 4.745 | −8.201 | 26.644 | 1.00 | 6.97 | A | O |
| ATOM | 1856 | N | GLN | A | 532 | 5.797 | −6.711 | 27.911 | 1.00 | 7.33 | A | N |
| ATOM | 1858 | CA | GLN | A | 532 | 4.706 | −5.735 | 27.831 | 1.00 | 7.02 | A | C |
| ATOM | 1860 | CB | GLN | A | 532 | 5.050 | −4.514 | 28.702 | 1.00 | 6.93 | A | C |
| ATOM | 1863 | CG | GLN | A | 532 | 4.930 | −4.815 | 30.195 | 1.00 | 7.50 | A | C |
| ATOM | 1866 | CD | GLN | A | 532 | 5.235 | −3.632 | 31.101 | 1.00 | 9.98 | A | C |
| ATOM | 1867 | OE1 | GLN | A | 532 | 5.756 | −2.626 | 30.657 | 1.00 | 9.61 | A | O |
| ATOM | 1868 | NE2 | GLN | A | 532 | 4.903 | −3.772 | 32.393 | 1.00 | 8.99 | A | N |
| ATOM | 1871 | C | GLN | A | 532 | 4.439 | −5.290 | 26.388 | 1.00 | 7.09 | A | C |
| ATOM | 1872 | O | GLN | A | 532 | 3.281 | −5.223 | 25.942 | 1.00 | 6.73 | A | O |
| ATOM | 1873 | N | ILE | A | 533 | 5.505 | −4.991 | 25.645 | 1.00 | 7.80 | A | N |
| ATOM | 1875 | CA | ILE | A | 533 | 5.341 | −4.572 | 24.264 | 1.00 | 8.29 | A | C |
| ATOM | 1877 | CB | ILE | A | 533 | 6.663 | −4.028 | 23.692 | 1.00 | 8.97 | A | C |
| ATOM | 1879 | CG1 | ILE | A | 533 | 7.124 | −2.779 | 24.445 | 1.00 | 8.38 | A | C |
| ATOM | 1882 | CD1 | ILE | A | 533 | 6.158 | −1.630 | 24.424 | 1.00 | 9.69 | A | C |
| ATOM | 1886 | CG2 | ILE | A | 533 | 6.527 | −3.760 | 22.221 | 1.00 | 9.41 | A | C |
| ATOM | 1890 | C | ILE | A | 533 | 4.825 | −5.736 | 23.419 | 1.00 | 8.33 | A | C |
| ATOM | 1891 | O | ILE | A | 533 | 4.010 | −5.548 | 22.512 | 1.00 | 8.38 | A | O |
| ATOM | 1892 | N | CYS | A | 534 | 5.305 | −6.938 | 23.717 | 1.00 | 7.80 | A | N |
| ATOM | 1894 | CA | CYS | A | 534 | 4.829 | −8.141 | 23.044 | 1.00 | 8.73 | A | C |
| ATOM | 1896 | CB | CYS | A | 534 | 5.588 | −9.373 | 23.521 | 1.00 | 9.35 | A | C |
| ATOM | 1899 | SG | CYS | A | 534 | 5.440 | −10.785 | 22.388 | 1.00 | 10.44 | A | S |
| ATOM | 1900 | C | CYS | A | 534 | 3.332 | −8.326 | 23.234 | 1.00 | 8.30 | A | C |
| ATOM | 1901 | O | CYS | A | 534 | 2.638 | −8.690 | 22.279 | 1.00 | 7.97 | A | O |
| ATOM | 1902 | N | LYS | A | 535 | 2.821 | −8.083 | 24.451 | 1.00 | 8.09 | A | N |
| ATOM | 1904 | CA | LYS | A | 535 | 1.381 | −8.179 | 24.685 | 1.00 | 8.26 | A | C |
| ATOM | 1906 | CB | LYS | A | 535 | 1.055 | −8.053 | 26.169 | 1.00 | 8.37 | A | C |
| ATOM | 1909 | CG | LYS | A | 535 | 1.491 | −9.239 | 26.965 | 1.00 | 10.48 | A | C |
| ATOM | 1912 | CD | LYS | A | 535 | 1.166 | −9.072 | 28.438 | 1.00 | 13.12 | A | C |
| ATOM | 1915 | CE | LYS | A | 535 | 1.380 | −10.378 | 29.159 | 1.00 | 15.75 | A | C |
| ATOM | 1918 | NZ | LYS | A | 535 | 1.022 | −10.255 | 30.588 | 1.00 | 19.99 | A | N |
| ATOM | 1922 | C | LYS | A | 535 | 0.569 | −7.160 | 23.880 | 1.00 | 8.96 | A | C |
| ATOM | 1923 | O | LYS | A | 535 | −0.525 | −7.471 | 23.383 | 1.00 | 8.89 | A | O |
| ATOM | 1924 | N | ALA | A | 536 | 1.079 | −5.946 | 23.758 | 1.00 | 8.31 | A | N |
| ATOM | 1926 | CA | ALA | A | 536 | 0.461 | −4.964 | 22.870 | 1.00 | 8.35 | A | C |
| ATOM | 1928 | CB | ALA | A | 536 | 1.208 | −3.652 | 22.932 | 1.00 | 8.92 | A | C |
| ATOM | 1932 | C | ALA | A | 536 | 0.418 | −5.463 | 21.434 | 1.00 | 8.28 | A | C |
| ATOM | 1933 | O | ALA | A | 536 | −0.595 | −5.288 | 20.745 | 1.00 | 8.64 | A | O |
| ATOM | 1934 | N | MET | A | 537 | 1.505 | −6.073 | 20.973 | 1.00 | 7.09 | A | N |
| ATOM | 1936 | CA | MET | A | 537 | 1.578 | −6.552 | 19.596 | 1.00 | 7.42 | A | C |
| ATOM | 1938 | CB | MET | A | 537 | 3.020 | −6.875 | 19.183 | 1.00 | 7.57 | A | C |
| ATOM | 1941 | CG | MET | A | 537 | 3.879 | −5.632 | 19.008 | 1.00 | 7.90 | A | C |
| ATOM | 1944 | SD | MET | A | 537 | 3.169 | −4.342 | 17.962 | 1.00 | 11.76 | A | S |
| ATOM | 1945 | CE | MET | A | 537 | 2.724 | −5.210 | 16.517 | 1.00 | 14.61 | A | C |
| ATOM | 1949 | C | MET | A | 537 | 0.679 | −7.755 | 19.365 | 1.00 | 7.65 | A | C |
| ATOM | 1950 | O | MET | A | 537 | 0.122 | −7.907 | 18.271 | 1.00 | 8.38 | A | O |
| ATOM | 1951 | N | ALA | A | 538 | 0.479 | −8.576 | 20.386 | 1.00 | 7.79 | A | N |
| ATOM | 1953 | CA | ALA | A | 538 | −0.451 | −9.717 | 20.244 | 1.00 | 8.20 | A | C |
| ATOM | 1955 | CB | ALA | A | 538 | −0.415 | −10.622 | 21.450 | 1.00 | 8.47 | A | C |
| ATOM | 1959 | C | ALA | A | 538 | −1.876 | −9.203 | 20.011 | 1.00 | 8.12 | A | C |
| ATOM | 1960 | O | ALA | A | 538 | −2.643 | −9.800 | 19.251 | 1.00 | 8.25 | A | O |
| ATOM | 1961 | N | TYR | A | 539 | −2.230 | −8.116 | 20.670 | 1.00 | 8.11 | A | N |
| ATOM | 1963 | CA | TYR | A | 539 | −3.542 | −7.483 | 20.457 | 1.00 | 9.32 | A | C |
| ATOM | 1965 | CB | TYR | A | 539 | −3.774 | −6.365 | 21.469 | 1.00 | 9.99 | A | C |
| ATOM | 1968 | CG | TYR | A | 539 | −5.068 | −5.630 | 21.207 | 1.00 | 11.93 | A | C |
| ATOM | 1969 | CD1 | TYR | A | 539 | −6.271 | −6.313 | 21.198 | 1.00 | 15.15 | A | C |
| ATOM | 1971 | CE1 | TYR | A | 539 | −7.490 | −5.653 | 20.916 | 1.00 | 19.59 | A | C |
| ATOM | 1973 | CZ | TYR | A | 539 | −7.490 | −4.300 | 20.654 | 1.00 | 21.53 | A | C |
| ATOM | 1974 | OH | TYR | A | 539 | −8.702 | −3.667 | 20.376 | 1.00 | 25.74 | A | O |
| ATOM | 1976 | CE2 | TYR | A | 539 | −6.300 | −3.595 | 20.656 | 1.00 | 19.80 | A | C |
| ATOM | 1978 | CD2 | TYR | A | 539 | −5.082 | −4.267 | 20.928 | 1.00 | 17.80 | A | C |
| ATOM | 1980 | C | TYR | A | 539 | −3.671 | −6.953 | 19.013 | 1.00 | 9.25 | A | C |
| ATOM | 1981 | O | TYR | A | 539 | −4.671 | −7.228 | 18.327 | 1.00 | 10.22 | A | O |
| ATOM | 1982 | N | LEU | A | 540 | −2.662 | −6.226 | 18.534 | 1.00 | 8.68 | A | N |
| ATOM | 1984 | CA | LEU | A | 540 | −2.681 | −5.747 | 17.162 | 1.00 | 9.52 | A | C |
| ATOM | 1986 | CB | LEU | A | 540 | −1.497 | −4.781 | 16.902 | 1.00 | 9.52 | A | C |
| ATOM | 1989 | CG | LEU | A | 540 | −1.503 | −3.503 | 17.757 | 1.00 | 9.17 | A | C |
| ATOM | 1991 | CD1 | LEU | A | 540 | −0.301 | −2.603 | 17.427 | 1.00 | 12.37 | A | C |
| ATOM | 1995 | CD2 | LEU | A | 540 | −2.795 | −2.746 | 17.652 | 1.00 | 11.42 | A | C |
| ATOM | 1999 | C | LEU | A | 540 | −2.765 | −6.896 | 16.132 | 1.00 | 10.02 | A | C |
| ATOM | 2000 | O | LEU | A | 540 | −3.496 | −6.801 | 15.139 | 1.00 | 10.77 | A | O |
| ATOM | 2001 | N | GLU | A | 541 | −2.053 | −7.983 | 16.386 | 1.00 | 9.88 | A | N |
| ATOM | 2003 | CA | GLU | A | 541 | −2.071 | −9.173 | 15.534 | 1.00 | 10.37 | A | C |
| ATOM | 2005 | CB | GLU | A | 541 | −1.081 | −10.216 | 16.087 | 1.00 | 10.24 | A | C |
| ATOM | 2008 | CG | GLU | A | 541 | −1.140 | −11.612 | 15.478 | 1.00 | 11.36 | A | C |
| ATOM | 2011 | CD | GLU | A | 541 | −0.136 | −12.569 | 16.101 | 1.00 | 10.66 | A | C |
| ATOM | 2012 | OE1 | GLU | A | 541 | −0.448 | −13.191 | 17.153 | 1.00 | 11.76 | A | O |
| ATOM | 2013 | OE2 | GLU | A | 541 | 0.968 | −12.704 | 15.543 | 1.00 | 11.66 | A | O |
| ATOM | 2014 | C | GLU | A | 541 | −3.491 | −9.739 | 15.437 | 1.00 | 10.85 | A | C |
| ATOM | 2015 | O | GLU | A | 541 | −3.907 | −10.192 | 14.378 | 1.00 | 11.81 | A | O |
| ATOM | 2016 | N | SER | A | 542 | −4.243 | −9.677 | 16.531 | 1.00 | 11.36 | A | N |
| ATOM | 2018 | CA | SER | A | 542 | −5.608 | −10.206 | 16.550 | 1.00 | 11.65 | A | C |
| ATOM | 2020 | CB | SER | A | 542 | −6.160 | −10.250 | 17.976 | 1.00 | 12.06 | A | C |
| ATOM | 2023 | OG | SER | A | 542 | −6.549 | −8.983 | 18.468 | 1.00 | 11.97 | A | O |
| ATOM | 2025 | C | SER | A | 542 | −6.576 | −9.458 | 15.641 | 1.00 | 12.56 | A | C |
| ATOM | 2026 | O | SER | A | 542 | −7.631 | −10.010 | 15.277 | 1.00 | 11.49 | A | O |
| ATOM | 2027 | N | ILE | A | 543 | −6.260 | −8.210 | 15.323 | 1.00 | 12.89 | A | N |
| ATOM | 2029 | CA | ILE | A | 543 | −7.050 | −7.424 | 14.358 | 1.00 | 13.67 | A | C |
| ATOM | 2031 | CB | ILE | A | 543 | −7.562 | −6.115 | 14.994 | 1.00 | 14.02 | A | C |
| ATOM | 2033 | CG1 | ILE | A | 543 | −6.433 | −5.283 | 15.598 | 1.00 | 14.22 | A | C |
| ATOM | 2036 | CD1 | ILE | A | 543 | −6.826 | −3.900 | 15.926 | 1.00 | 15.54 | A | C |
| ATOM | 2040 | CG2 | ILE | A | 543 | −8.591 | −6.404 | 16.074 | 1.00 | 13.84 | A | C |
| ATOM | 2044 | C | ILE | A | 543 | −6.317 | −7.159 | 13.042 | 1.00 | 14.47 | A | C |
| ATOM | 2045 | O | ILE | A | 543 | −6.732 | −6.316 | 12.252 | 1.00 | 14.15 | A | O |
| ATOM | 2046 | N | ASN | A | 544 | −5.235 | −7.885 | 12.809 | 1.00 | 15.25 | A | N |
| ATOM | 2048 | CA | ASN | A | 544 | −4.457 | −7.780 | 11.575 | 1.00 | 17.01 | A | C |
| ATOM | 2050 | CB | ASN | A | 544 | −5.260 | −8.311 | 10.368 | 1.00 | 18.38 | A | C |
| ATOM | 2053 | CG | ASN | A | 544 | −5.668 | −9.758 | 10.523 | 1.00 | 22.26 | A | C |
| ATOM | 2054 | OD1 | ASN | A | 544 | −4.821 | −10.644 | 10.660 | 1.00 | 28.79 | A | O |
| ATOM | 2055 | ND2 | ASN | A | 544 | −6.970 | −10.012 | 10.491 | 1.00 | 27.53 | A | N |
| ATOM | 2058 | C | ASN | A | 544 | −3.995 | −6.353 | 11.302 | 1.00 | 17.05 | A | C |
| ATOM | 2059 | O | ASN | A | 544 | −4.019 | −5.878 | 10.162 | 1.00 | 18.03 | A | O |
| ATOM | 2060 | N | CYS | A | 545 | −3.591 | −5.672 | 12.365 | 1.00 | 16.00 | A | N |
| ATOM | 2062 | CA | CYS | A | 545 | −3.103 | −4.305 | 12.291 | 1.00 | 16.72 | A | C |
| ATOM | 2064 | CB | CYS | A | 545 | −3.628 | −3.544 | 13.494 | 1.00 | 16.76 | A | C |
| ATOM | 2067 | SG | CYS | A | 545 | −3.019 | −1.887 | 13.701 | 1.00 | 24.49 | A | S |
| ATOM | 2068 | C | CYS | A | 545 | −1.586 | −4.358 | 12.295 | 1.00 | 15.33 | A | C |
| ATOM | 2069 | O | CYS | A | 545 | −0.994 | −4.800 | 13.278 | 1.00 | 15.78 | A | O |
| ATOM | 2070 | N | VAL | A | 546 | −0.969 | −3.933 | 11.194 | 1.00 | 14.28 | A | N |
| ATOM | 2072 | CA | VAL | A | 546 | 0.498 | −3.913 | 11.064 | 1.00 | 13.55 | A | C |
| ATOM | 2074 | CB | VAL | A | 546 | 0.939 | −4.206 | 9.610 | 1.00 | 14.14 | A | C |
| ATOM | 2076 | CG1 | VAL | A | 546 | 2.466 | −4.261 | 9.507 | 1.00 | 15.81 | A | C |
| ATOM | 2080 | CG2 | VAL | A | 546 | 0.296 | −5.520 | 9.111 | 1.00 | 15.91 | A | C |
| ATOM | 2084 | C | VAL | A | 546 | 0.998 | −2.542 | 11.524 | 1.00 | 13.04 | A | C |
| ATOM | 2085 | O | VAL | A | 546 | 0.606 | −1.505 | 10.980 | 1.00 | 12.91 | A | O |
| ATOM | 2086 | N | HIS | A | 547 | 1.871 | −2.540 | 12.533 | 1.00 | 11.11 | A | N |
| ATOM | 2088 | CA | HIS | A | 547 | 2.287 | −1.318 | 13.227 | 1.00 | 10.91 | A | C |
| ATOM | 2090 | CB | HIS | A | 547 | 2.643 | −1.668 | 14.683 | 1.00 | 10.19 | A | C |
| ATOM | 2093 | CG | HIS | A | 547 | 2.902 | −0.475 | 15.544 | 1.00 | 10.34 | A | C |
| ATOM | 2094 | ND1 | HIS | A | 547 | 4.081 | 0.228 | 15.491 | 1.00 | 11.48 | A | N |
| ATOM | 2096 | CE1 | HIS | A | 547 | 4.038 | 1.226 | 16.361 | 1.00 | 10.70 | A | C |
| ATOM | 2098 | NE2 | HIS | A | 547 | 2.864 | 1.199 | 16.967 | 1.00 | 9.34 | A | N |
| ATOM | 2100 | CD2 | HIS | A | 547 | 2.140 | 0.138 | 16.481 | 1.00 | 10.35 | A | C |
| ATOM | 2102 | C | HIS | A | 547 | 3.431 | −0.584 | 12.527 | 1.00 | 10.89 | A | C |
| ATOM | 2103 | O | HIS | A | 547 | 3.342 | 0.628 | 12.322 | 1.00 | 11.28 | A | O |
| ATOM | 2104 | N | ARG | A | 548 | 4.479 | −1.321 | 12.157 | 1.00 | 10.62 | A | N |
| ATOM | 2106 | CA | ARG | A | 548 | 5.652 | −0.819 | 11.406 | 1.00 | 10.76 | A | C |
| ATOM | 2108 | CB | ARG | A | 548 | 5.248 | −0.092 | 10.114 | 1.00 | 11.42 | A | C |
| ATOM | 2111 | CG | ARG | A | 548 | 4.346 | −0.845 | 9.128 | 1.00 | 12.89 | A | C |
| ATOM | 2114 | CD | ARG | A | 548 | 4.246 | −0.093 | 7.781 | 1.00 | 15.96 | A | C |
| ATOM | 2117 | NE | ARG | A | 548 | 3.297 | −0.686 | 6.841 | 1.00 | 16.19 | A | N |
| ATOM | 2119 | CZ | ARG | A | 548 | 3.236 | −0.367 | 5.543 | 1.00 | 18.71 | A | C |
| ATOM | 2120 | NH1 | ARG | A | 548 | 4.045 | 0.549 | 5.039 | 1.00 | 17.01 | A | N |
| ATOM | 2123 | NH2 | ARG | A | 548 | 2.350 | −0.948 | 4.752 | 1.00 | 19.52 | A | N |
| ATOM | 2126 | C | ARG | A | 548 | 6.616 | 0.100 | 12.165 | 1.00 | 10.94 | A | C |
| ATOM | 2127 | O | ARG | A | 548 | 7.610 | 0.540 | 11.585 | 1.00 | 11.17 | A | O |
| ATOM | 2128 | N | ASP | A | 549 | 6.345 | 0.423 | 13.421 | 1.00 | 10.64 | A | N |
| ATOM | 2130 | CA | ASP | A | 549 | 7.255 | 1.294 | 14.181 | 1.00 | 11.41 | A | C |
| ATOM | 2132 | CB | ASP | A | 549 | 6.824 | 2.767 | 14.120 | 1.00 | 11.59 | A | C |
| ATOM | 2135 | CG | ASP | A | 549 | 7.947 | 3.735 | 14.497 | 1.00 | 16.46 | A | C |
| ATOM | 2136 | OD1 | ASP | A | 549 | 9.129 | 3.319 | 14.543 | 1.00 | 19.23 | A | O |
| ATOM | 2137 | OD2 | ASP | A | 549 | 7.726 | 4.944 | 14.778 | 1.00 | 18.02 | A | O |
| ATOM | 2138 | C | ASP | A | 549 | 7.456 | 0.836 | 15.616 | 1.00 | 10.41 | A | C |
| ATOM | 2139 | O | ASP | A | 549 | 7.269 | 1.597 | 16.576 | 1.00 | 10.29 | A | O |
| ATOM | 2140 | N | ILE | A | 550 | 7.926 | −0.400 | 15.743 | 1.00 | 10.29 | A | N |
| ATOM | 2142 | CA | ILE | A | 550 | 8.229 | −0.986 | 17.046 | 1.00 | 10.14 | A | C |
| ATOM | 2144 | CB | ILE | A | 550 | 7.858 | −2.490 | 17.037 | 1.00 | 10.18 | A | C |
| ATOM | 2146 | CG1 | ILE | A | 550 | 6.440 | −2.685 | 16.476 | 1.00 | 9.59 | A | C |
| ATOM | 2149 | CD1 | ILE | A | 550 | 6.199 | −4.070 | 15.917 | 1.00 | 12.93 | A | C |
| ATOM | 2153 | CG2 | ILE | A | 550 | 7.936 | −3.059 | 18.435 | 1.00 | 10.80 | A | C |
| ATOM | 2157 | C | ILE | A | 550 | 9.709 | −0.791 | 17.310 | 1.00 | 10.07 | A | C |
| ATOM | 2158 | O | ILE | A | 550 | 10.517 | −1.517 | 16.793 | 1.00 | 10.23 | A | O |
| ATOM | 2159 | N | ALA | A | 551 | 10.032 | 0.227 | 18.094 | 1.00 | 10.23 | A | N |
| ATOM | 2161 | CA | ALA | A | 551 | 11.403 | 0.671 | 18.312 | 1.00 | 9.79 | A | C |
| ATOM | 2163 | CB | ALA | A | 551 | 11.883 | 1.526 | 17.130 | 1.00 | 9.99 | A | C |
| ATOM | 2167 | C | ALA | A | 551 | 11.404 | 1.495 | 19.593 | 1.00 | 9.51 | A | C |
| ATOM | 2168 | O | ALA | A | 551 | 10.399 | 2.111 | 19.911 | 1.00 | 8.53 | A | O |
| ATOM | 2169 | N | VAL | A | 552 | 12.536 | 1.529 | 20.304 | 1.00 | 8.79 | A | N |
| ATOM | 2171 | CA | VAL | A | 552 | 12.595 | 2.169 | 21.630 | 1.00 | 9.71 | A | C |
| ATOM | 2173 | CB | VAL | A | 552 | 13.917 | 1.852 | 22.434 | 1.00 | 9.32 | A | C |
| ATOM | 2175 | CG1 | VAL | A | 552 | 13.981 | 0.377 | 22.814 | 1.00 | 11.97 | A | C |
| ATOM | 2179 | CG2 | VAL | A | 552 | 15.175 | 2.296 | 21.700 | 1.00 | 10.51 | A | C |
| ATOM | 2183 | C | VAL | A | 552 | 12.373 | 3.671 | 21.605 | 1.00 | 9.86 | A | C |
| ATOM | 2184 | O | VAL | A | 552 | 11.910 | 4.231 | 22.605 | 1.00 | 9.34 | A | O |
| ATOM | 2185 | N | ARG | A | 553 | 12.672 | 4.305 | 20.462 | 1.00 | 9.93 | A | N |
| ATOM | 2187 | CA | ARG | A | 553 | 12.381 | 5.715 | 20.233 | 1.00 | 11.22 | A | C |
| ATOM | 2189 | CB | ARG | A | 553 | 12.914 | 6.152 | 18.849 | 1.00 | 12.32 | A | C |
| ATOM | 2192 | CG | ARG | A | 553 | 12.746 | 7.598 | 18.520 | 1.00 | 18.30 | A | C |
| ATOM | 2195 | CD | ARG | A | 553 | 13.541 | 8.051 | 17.284 | 1.00 | 25.13 | A | C |
| ATOM | 2198 | NE | ARG | A | 553 | 13.202 | 9.422 | 16.907 | 1.00 | 30.79 | A | N |
| ATOM | 2200 | CZ | ARG | A | 553 | 13.974 | 10.246 | 16.190 | 1.00 | 34.09 | A | C |
| ATOM | 2201 | NH1 | ARG | A | 553 | 15.161 | 9.865 | 15.721 | 1.00 | 35.69 | A | N |
| ATOM | 2204 | NH2 | ARG | A | 553 | 13.536 | 11.475 | 15.928 | 1.00 | 35.84 | A | N |
| ATOM | 2207 | C | ARG | A | 553 | 10.873 | 5.962 | 20.295 | 1.00 | 10.85 | A | C |
| ATOM | 2208 | O | ARG | A | 553 | 10.427 | 7.061 | 20.646 | 1.00 | 9.83 | A | O |
| ATOM | 2209 | N | ASN | A | 554 | 10.101 | 4.948 | 19.911 | 1.00 | 8.85 | A | N |
| ATOM | 2211 | CA | ASN | A | 554 | 8.650 | 5.050 | 19.820 | 1.00 | 9.54 | A | C |
| ATOM | 2213 | CB | ASN | A | 554 | 8.173 | 4.595 | 18.434 | 1.00 | 8.92 | A | C |
| ATOM | 2216 | CG | ASN | A | 554 | 6.729 | 4.998 | 18.148 | 1.00 | 10.60 | A | C |
| ATOM | 2217 | OD1 | ASN | A | 554 | 6.321 | 6.131 | 18.457 | 1.00 | 11.60 | A | O |
| ATOM | 2218 | ND2 | ASN | A | 554 | 5.959 | 4.101 | 17.560 | 1.00 | 11.34 | A | N |
| ATOM | 2221 | C | ASN | A | 554 | 7.907 | 4.279 | 20.910 | 1.00 | 9.30 | A | C |
| ATOM | 2222 | O | ASN | A | 554 | 6.788 | 3.841 | 20.707 | 1.00 | 10.60 | A | O |
| ATOM | 2223 | N | ILE | A | 555 | 8.544 | 4.108 | 22.062 | 1.00 | 10.21 | A | N |
| ATOM | 2225 | CA | ILE | A | 555 | 7.935 | 3.514 | 23.235 | 1.00 | 9.86 | A | C |
| ATOM | 2227 | CB | ILE | A | 555 | 8.595 | 2.177 | 23.597 | 1.00 | 9.61 | A | C |
| ATOM | 2229 | CG1 | ILE | A | 555 | 8.358 | 1.140 | 22.488 | 1.00 | 10.41 | A | C |
| ATOM | 2232 | CD1 | ILE | A | 555 | 9.318 | 0.017 | 22.555 | 1.00 | 11.60 | A | C |
| ATOM | 2236 | CG2 | ILE | A | 555 | 8.103 | 1.672 | 24.948 | 1.00 | 9.20 | A | C |
| ATOM | 2240 | C | ILE | A | 555 | 8.114 | 4.536 | 24.350 | 1.00 | 10.75 | A | C |
| ATOM | 2241 | O | ILE | A | 555 | 9.208 | 5.071 | 24.541 | 1.00 | 10.55 | A | O |
| ATOM | 2242 | N | LEU | A | 556 | 7.044 | 4.807 | 25.088 | 1.00 | 10.73 | A | N |
| ATOM | 2244 | CA | LEU | A | 556 | 7.084 | 5.751 | 26.209 | 1.00 | 11.14 | A | C |
| ATOM | 2246 | CB | LEU | A | 556 | 5.932 | 6.765 | 26.100 | 1.00 | 11.83 | A | C |
| ATOM | 2249 | CG | LEU | A | 556 | 6.005 | 7.814 | 24.986 | 1.00 | 14.83 | A | C |
| ATOM | 2251 | CD1 | LEU | A | 556 | 7.308 | 8.567 | 24.986 | 1.00 | 18.18 | A | C |
| ATOM | 2255 | CD2 | LEU | A | 556 | 5.770 | 7.191 | 23.644 | 1.00 | 19.54 | A | C |
| ATOM | 2259 | C | LEU | A | 556 | 7.001 | 5.052 | 27.535 | 1.00 | 10.30 | A | C |
| ATOM | 2260 | O | LEU | A | 556 | 6.353 | 4.017 | 27.670 | 1.00 | 9.91 | A | O |
| ATOM | 2261 | N | VAL | A | 557 | 7.636 | 5.660 | 28.529 | 1.00 | 9.82 | A | N |
| ATOM | 2263 | CA | VAL | A | 557 | 7.790 | 5.088 | 29.856 | 1.00 | 10.20 | A | C |
| ATOM | 2265 | CB | VAL | A | 557 | 9.244 | 5.235 | 30.379 | 1.00 | 9.42 | A | C |
| ATOM | 2267 | CG1 | VAL | A | 557 | 9.382 | 4.662 | 31.776 | 1.00 | 9.30 | A | C |
| ATOM | 2271 | CG2 | VAL | A | 557 | 10.274 | 4.537 | 29.435 | 1.00 | 10.82 | A | C |
| ATOM | 2275 | C | VAL | A | 557 | 6.809 | 5.808 | 30.788 | 1.00 | 10.92 | A | C |
| ATOM | 2276 | O | VAL | A | 557 | 6.990 | 6.979 | 31.126 | 1.00 | 10.29 | A | O |
| ATOM | 2277 | N | ALA | A | 558 | 5.744 | 5.103 | 31.149 | 1.00 | 11.83 | A | N |
| ATOM | 2279 | CA | ALA | A | 558 | 4.753 | 5.595 | 32.116 | 1.00 | 12.73 | A | C |
| ATOM | 2281 | CB | ALA | A | 558 | 3.474 | 4.716 | 32.040 | 1.00 | 12.91 | A | C |
| ATOM | 2285 | C | ALA | A | 558 | 5.291 | 5.615 | 33.542 | 1.00 | 13.27 | A | C |
| ATOM | 2286 | O | ALA | A | 558 | 5.043 | 6.550 | 34.330 | 1.00 | 14.71 | A | O |
| ATOM | 2287 | N | SER | A | 559 | 6.010 | 4.559 | 33.884 | 1.00 | 13.11 | A | N |
| ATOM | 2289 | CA | SER | A | 559 | 6.639 | 4.396 | 35.186 | 1.00 | 13.68 | A | C |
| ATOM | 2291 | CB | SER | A | 559 | 5.618 | 3.934 | 36.220 | 1.00 | 13.21 | A | C |
| ATOM | 2294 | OG | SER | A | 559 | 5.230 | 2.589 | 36.006 | 1.00 | 13.73 | A | O |
| ATOM | 2296 | C | SER | A | 559 | 7.719 | 3.352 | 35.030 | 1.00 | 13.54 | A | C |
| ATOM | 2297 | O | SER | A | 559 | 7.737 | 2.663 | 34.025 | 1.00 | 12.55 | A | O |
| ATOM | 2298 | N | PRO | A | 560 | 8.593 | 3.177 | 36.018 | 1.00 | 14.61 | A | N |
| ATOM | 2299 | CA | PRO | A | 560 | 9.559 | 2.069 | 35.950 | 1.00 | 15.41 | A | C |
| ATOM | 2301 | CB | PRO | A | 560 | 10.361 | 2.217 | 37.251 | 1.00 | 15.46 | A | C |
| ATOM | 2304 | CG | PRO | A | 560 | 10.251 | 3.662 | 37.572 | 1.00 | 15.49 | A | C |
| ATOM | 2307 | CD | PRO | A | 560 | 8.794 | 3.996 | 37.233 | 1.00 | 15.51 | A | C |
| ATOM | 2310 | C | PRO | A | 560 | 8.919 | 0.678 | 35.800 | 1.00 | 15.83 | A | C |
| ATOM | 2311 | O | PRO | A | 560 | 9.589 | −0.279 | 35.390 | 1.00 | 16.34 | A | O |
| ATOM | 2312 | N | GLU | A | 561 | 7.626 | 0.579 | 36.070 | 1.00 | 16.53 | A | N |
| ATOM | 2314 | CA | GLU | A | 561 | 6.918 | −0.689 | 35.993 | 1.00 | 16.63 | A | C |
| ATOM | 2316 | CB | GLU | A | 561 | 6.035 | −0.834 | 37.234 | 1.00 | 18.01 | A | C |
| ATOM | 2319 | CG | GLU | A | 561 | 6.833 | −0.946 | 38.529 | 1.00 | 22.37 | A | C |
| ATOM | 2322 | CD | GLU | A | 561 | 6.517 | 0.168 | 39.506 | 1.00 | 28.24 | A | C |
| ATOM | 2323 | OE1 | GLU | A | 561 | 6.453 | 1.357 | 39.072 | 1.00 | 32.07 | A | O |
| ATOM | 2324 | OE2 | GLU | A | 561 | 6.315 | −0.152 | 40.707 | 1.00 | 33.30 | A | O |
| ATOM | 2325 | C | GLU | A | 561 | 6.059 | −0.871 | 34.733 | 1.00 | 14.93 | A | C |
| ATOM | 2326 | O | GLU | A | 561 | 5.445 | −1.916 | 34.574 | 1.00 | 14.59 | A | O |
| ATOM | 2327 | N | CYS | A | 562 | 6.025 | 0.111 | 33.835 | 1.00 | 13.75 | A | N |
| ATOM | 2329 | CA | CYS | A | 562 | 5.074 | 0.089 | 32.711 | 1.00 | 14.08 | A | C |
| ATOM | 2331 | CB | CYS | A | 562 | 3.692 | 0.541 | 33.159 | 1.00 | 14.16 | A | C |
| ATOM | 2334 | SG | CYS | A | 562 | 2.464 | 0.485 | 31.846 | 1.00 | 17.94 | A | S |
| ATOM | 2335 | C | CYS | A | 562 | 5.534 | 0.942 | 31.520 | 1.00 | 12.60 | A | C |
| ATOM | 2336 | O | CYS | A | 562 | 5.747 | 2.155 | 31.640 | 1.00 | 12.45 | A | O |
| ATOM | 2337 | N | VAL | A | 563 | 5.709 | 0.282 | 30.377 | 1.00 | 11.25 | A | N |
| ATOM | 2339 | CA | VAL | A | 563 | 5.979 | 0.962 | 29.125 | 1.00 | 10.38 | A | C |
| ATOM | 2341 | CB | VAL | A | 563 | 7.259 | 0.421 | 28.421 | 1.00 | 10.60 | A | C |
| ATOM | 2343 | CG1 | VAL | A | 563 | 8.477 | 0.548 | 29.329 | 1.00 | 10.27 | A | C |
| ATOM | 2347 | CG2 | VAL | A | 563 | 7.102 | −1.020 | 27.949 | 1.00 | 10.55 | A | C |
| ATOM | 2351 | C | VAL | A | 563 | 4.748 | 0.912 | 28.184 | 1.00 | 10.67 | A | C |
| ATOM | 2352 | O | VAL | A | 563 | 3.867 | 0.058 | 28.336 | 1.00 | 10.13 | A | O |
| ATOM | 2353 | N | LYS | A | 564 | 4.723 | 1.806 | 27.202 | 1.00 | 9.96 | A | N |
| ATOM | 2355 | CA | LYS | A | 564 | 3.586 | 1.997 | 26.285 | 1.00 | 10.71 | A | C |
| ATOM | 2357 | CB | LYS | A | 564 | 2.835 | 3.290 | 26.642 | 1.00 | 10.90 | A | C |
| ATOM | 2360 | CG | LYS | A | 564 | 2.453 | 3.420 | 28.104 | 1.00 | 13.45 | A | C |
| ATOM | 2363 | CD | LYS | A | 564 | 1.102 | 2.850 | 28.403 | 1.00 | 14.37 | A | C |
| ATOM | 2366 | CE | LYS | A | 564 | 0.676 | 3.158 | 29.838 | 1.00 | 16.32 | A | C |
| ATOM | 2369 | NZ | LYS | A | 564 | −0.589 | 2.458 | 30.221 | 1.00 | 17.26 | A | N |
| ATOM | 2373 | C | LYS | A | 564 | 4.050 | 2.148 | 24.828 | 1.00 | 9.71 | A | C |
| ATOM | 2374 | O | LYS | A | 564 | 4.814 | 3.069 | 24.502 | 1.00 | 9.88 | A | O |
| ATOM | 2375 | N | LEU | A | 565 | 3.615 | 1.238 | 23.964 | 1.00 | 8.95 | A | N |
| ATOM | 2377 | CA | LEU | A | 565 | 3.909 | 1.334 | 22.533 | 1.00 | 9.28 | A | C |
| ATOM | 2379 | CB | LEU | A | 565 | 3.418 | 0.094 | 21.802 | 1.00 | 9.45 | A | C |
| ATOM | 2382 | CG | LEU | A | 565 | 3.702 | 0.004 | 20.289 | 1.00 | 9.53 | A | C |
| ATOM | 2384 | CD1 | LEU | A | 565 | 5.202 | 0.062 | 20.003 | 1.00 | 10.72 | A | C |
| ATOM | 2388 | CD2 | LEU | A | 565 | 3.070 | −1.258 | 19.781 | 1.00 | 12.45 | A | C |
| ATOM | 2392 | C | LEU | A | 565 | 3.245 | 2.565 | 21.928 | 1.00 | 9.46 | A | C |
| ATOM | 2393 | O | LEU | A | 565 | 2.054 | 2.825 | 22.167 | 1.00 | 9.53 | A | O |
| ATOM | 2394 | N | GLY | A | 566 | 4.007 | 3.302 | 21.131 | 1.00 | 10.94 | A | N |
| ATOM | 2396 | CA | GLY | A | 566 | 3.540 | 4.539 | 20.511 | 1.00 | 12.38 | A | C |
| ATOM | 2399 | C | GLY | A | 566 | 2.748 | 4.337 | 19.222 | 1.00 | 14.40 | A | C |
| ATOM | 2400 | O | GLY | A | 566 | 2.312 | 3.235 | 18.882 | 1.00 | 12.68 | A | O |
| ATOM | 2401 | N | ASP | A | 567 | 2.524 | 5.424 | 18.494 | 1.00 | 17.06 | A | N |
| ATOM | 2403 | CA | ASP | A | 567 | 1.644 | 5.322 | 17.327 | 1.00 | 19.73 | A | C |
| ATOM | 2405 | CB | ASP | A | 567 | 0.986 | 6.649 | 16.942 | 1.00 | 21.13 | A | C |
| ATOM | 2408 | CG | ASP | A | 567 | 1.917 | 7.779 | 16.889 | 1.00 | 24.80 | A | C |
| ATOM | 2409 | OD1 | ASP | A | 567 | 2.981 | 7.672 | 16.231 | 1.00 | 32.54 | A | O |
| ATOM | 2410 | OD2 | ASP | A | 567 | 1.623 | 8.856 | 17.439 | 1.00 | 29.95 | A | O |
| ATOM | 2411 | C | ASP | A | 567 | 2.257 | 4.622 | 16.108 | 1.00 | 20.41 | A | C |
| ATOM | 2412 | O | ASP | A | 567 | 3.467 | 4.413 | 16.011 | 1.00 | 17.31 | A | O |
| ATOM | 2413 | N | PHE | A | 568 | 1.368 | 4.262 | 15.196 | 1.00 | 22.78 | A | N |
| ATOM | 2415 | CA | PHE | A | 568 | 1.698 | 3.525 | 13.979 | 1.00 | 25.32 | A | C |
| ATOM | 2417 | CB | PHE | A | 568 | 0.428 | 3.274 | 13.152 | 1.00 | 26.08 | A | C |
| ATOM | 2420 | CG | PHE | A | 568 | −0.619 | 2.555 | 13.894 | 1.00 | 27.54 | A | C |
| ATOM | 2421 | CD1 | PHE | A | 568 | −1.780 | 3.199 | 14.285 | 1.00 | 29.54 | A | C |
| ATOM | 2423 | CE1 | PHE | A | 568 | −2.745 | 2.522 | 14.983 | 1.00 | 29.57 | A | C |
| ATOM | 2425 | CZ | PHE | A | 568 | −2.560 | 1.197 | 15.312 | 1.00 | 30.42 | A | C |
| ATOM | 2427 | CE2 | PHE | A | 568 | −1.412 | 0.548 | 14.935 | 1.00 | 30.12 | A | C |
| ATOM | 2429 | CD2 | PHE | A | 568 | −0.443 | 1.228 | 14.228 | 1.00 | 29.43 | A | C |
| ATOM | 2431 | C | PHE | A | 568 | 2.666 | 4.282 | 13.116 | 1.00 | 27.26 | A | C |
| ATOM | 2432 | O | PHE | A | 568 | 2.610 | 5.502 | 13.050 | 1.00 | 27.24 | A | O |
| ATOM | 2433 | N | GLY | A | 569 | 3.536 | 3.545 | 12.433 | 1.00 | 29.69 | A | N |
| ATOM | 2435 | CA | GLY | A | 569 | 4.511 | 4.124 | 11.531 | 1.00 | 32.08 | A | C |
| ATOM | 2438 | C | GLY | A | 569 | 3.937 | 5.058 | 10.482 | 1.00 | 34.61 | A | C |
| ATOM | 2439 | O | GLY | A | 569 | 4.602 | 6.029 | 10.110 | 1.00 | 34.79 | A | O |
| ATOM | 2440 | N | LEU | A | 570 | 2.724 | 4.754 | 10.010 | 1.00 | 37.79 | A | N |
| ATOM | 2442 | CA | LEU | A | 570 | 1.999 | 5.549 | 8.993 | 1.00 | 40.50 | A | C |
| ATOM | 2444 | CB | LEU | A | 570 | 0.514 | 5.665 | 9.387 | 1.00 | 40.83 | A | C |
| ATOM | 2447 | CG | LEU | A | 570 | −0.463 | 6.442 | 8.489 | 1.00 | 41.93 | A | C |
| ATOM | 2449 | CD1 | LEU | A | 570 | −0.487 | 5.925 | 7.051 | 1.00 | 42.79 | A | C |
| ATOM | 2453 | CD2 | LEU | A | 570 | −1.870 | 6.383 | 9.095 | 1.00 | 42.81 | A | C |
| ATOM | 2457 | C | LEU | A | 570 | 2.572 | 6.949 | 8.737 | 1.00 | 42.14 | A | C |
| ATOM | 2458 | O | LEU | A | 570 | 2.682 | 7.769 | 9.670 | 1.00 | 43.08 | A | O |
| ATOM | 2459 | N | SER | A | 571 | 2.929 | 7.207 | 7.475 | 1.00 | 43.89 | A | N |
| ATOM | 2461 | CA | SER | A | 571 | 3.588 | 8.455 | 7.062 | 1.00 | 45.06 | A | C |
| ATOM | 2463 | CB | SER | A | 571 | 4.686 | 8.163 | 6.017 | 1.00 | 45.02 | A | C |
| ATOM | 2466 | OG | SER | A | 571 | 5.526 | 7.091 | 6.423 | 1.00 | 45.78 | A | O |
| ATOM | 2468 | C | SER | A | 571 | 2.551 | 9.456 | 6.521 | 1.00 | 46.00 | A | C |
| ATOM | 2469 | O | SER | A | 571 | 1.345 | 9.295 | 6.762 | 1.00 | 46.79 | A | O |
| ATOM | 2470 | N | ARG | A | 572 | 3.012 | 10.481 | 5.800 | 1.00 | 46.90 | A | N |
| ATOM | 2472 | CA | ARG | A | 572 | 2.146 | 11.585 | 5.348 | 1.00 | 47.52 | A | C |
| ATOM | 2474 | CB | ARG | A | 572 | 2.414 | 12.856 | 6.181 | 1.00 | 47.67 | A | C |
| ATOM | 2477 | CG | ARG | A | 572 | 3.899 | 13.255 | 6.343 | 1.00 | 48.36 | A | C |
| ATOM | 2480 | CD | ARG | A | 572 | 4.392 | 14.299 | 5.342 | 1.00 | 48.94 | A | C |
| ATOM | 2483 | NE | ARG | A | 572 | 5.846 | 14.476 | 5.371 | 1.00 | 49.60 | A | N |
| ATOM | 2485 | CZ | ARG | A | 572 | 6.548 | 15.158 | 4.461 | 1.00 | 49.93 | A | C |
| ATOM | 2486 | NH1 | ARG | A | 572 | 5.942 | 15.748 | 3.432 | 1.00 | 49.93 | A | N |
| ATOM | 2489 | NH2 | ARG | A | 572 | 7.869 | 15.255 | 4.580 | 1.00 | 50.04 | A | N |
| ATOM | 2492 | C | ARG | A | 572 | 2.299 | 11.870 | 3.842 | 1.00 | 47.78 | A | C |
| ATOM | 2493 | O | ARG | A | 572 | 2.330 | 13.032 | 3.413 | 1.00 | 47.93 | A | O |
| ATOM | 2494 | N | TYR | A | 573 | 2.348 | 10.800 | 3.047 | 1.00 | 47.99 | A | N |
| ATOM | 2496 | CA | TYR | A | 573 | 2.606 | 10.890 | 1.604 | 1.00 | 48.13 | A | C |
| ATOM | 2498 | CB | TYR | A | 573 | 3.839 | 10.038 | 1.264 | 1.00 | 48.32 | A | C |
| ATOM | 2501 | CG | TYR | A | 573 | 5.090 | 10.843 | 0.971 | 1.00 | 49.01 | A | C |
| ATOM | 2502 | CD1 | TYR | A | 573 | 5.840 | 11.408 | 2.003 | 1.00 | 49.66 | A | C |
| ATOM | 2504 | CE1 | TYR | A | 573 | 6.988 | 12.154 | 1.736 | 1.00 | 50.12 | A | C |
| ATOM | 2506 | CZ | TYR | A | 573 | 7.396 | 12.335 | 0.421 | 1.00 | 50.65 | A | C |
| ATOM | 2507 | OH | TYR | A | 573 | 8.530 | 13.070 | 0.141 | 1.00 | 51.55 | A | O |
| ATOM | 2509 | CE2 | TYR | A | 573 | 6.668 | 11.784 | −0.618 | 1.00 | 50.16 | A | C |
| ATOM | 2511 | CD2 | TYR | A | 573 | 5.522 | 11.041 | −0.340 | 1.00 | 49.75 | A | C |
| ATOM | 2513 | C | TYR | A | 573 | 1.396 | 10.437 | 0.749 | 1.00 | 47.99 | A | C |
| ATOM | 2514 | O | TYR | A | 573 | 0.637 | 9.549 | 1.157 | 1.00 | 48.18 | A | O |
| ATOM | 2515 | N | ILE | A | 574 | 1.218 | 11.060 | −0.423 | 1.00 | 47.68 | A | N |
| ATOM | 2517 | CA | ILE | A | 574 | 0.221 | 10.611 | −1.414 | 1.00 | 47.28 | A | C |
| ATOM | 2519 | CB | ILE | A | 574 | −0.335 | 11.823 | −2.263 | 1.00 | 47.36 | A | C |
| ATOM | 2521 | CG1 | ILE | A | 574 | −1.852 | 11.681 | −2.521 | 1.00 | 47.24 | A | C |
| ATOM | 2524 | CD1 | ILE | A | 574 | −2.265 | 10.667 | −3.589 | 1.00 | 47.19 | A | C |
| ATOM | 2528 | CG2 | ILE | A | 574 | 0.468 | 12.038 | −3.572 | 1.00 | 47.44 | A | C |
| ATOM | 2532 | C | ILE | A | 574 | 0.819 | 9.524 | −2.312 | 1.00 | 46.58 | A | C |
| ATOM | 2533 | O | ILE | A | 574 | 0.097 | 8.640 | −2.793 | 1.00 | 47.04 | A | O |
| ATOM | 2534 | N | GLU | A | 575 | 2.133 | 9.598 | −2.539 | 1.00 | 45.52 | A | N |
| ATOM | 2536 | CA | GLU | A | 575 | 2.848 | 8.569 | −3.297 | 1.00 | 44.42 | A | C |
| ATOM | 2538 | CB | GLU | A | 575 | 4.131 | 9.120 | −3.949 | 1.00 | 44.66 | A | C |
| ATOM | 2541 | CG | GLU | A | 575 | 4.611 | 8.287 | −5.139 | 1.00 | 45.56 | A | C |
| ATOM | 2544 | CD | GLU | A | 575 | 5.484 | 9.057 | −6.128 | 1.00 | 47.20 | A | C |
| ATOM | 2545 | OE1 | GLU | A | 575 | 6.216 | 9.984 | −5.710 | 1.00 | 47.38 | A | O |
| ATOM | 2546 | OE2 | GLU | A | 575 | 5.450 | 8.721 | −7.335 | 1.00 | 47.58 | A | O |
| ATOM | 2547 | C | GLU | A | 575 | 3.162 | 7.383 | −2.392 | 1.00 | 42.62 | A | C |
| ATOM | 2548 | O | GLU | A | 575 | 2.802 | 6.252 | −2.726 | 1.00 | 43.25 | A | O |
| ATOM | 2549 | N | ASP | A | 576 | 3.840 | 7.644 | −1.266 | 1.00 | 40.22 | A | N |
| ATOM | 2551 | CA | ASP | A | 576 | 4.075 | 6.649 | −0.202 | 1.00 | 37.98 | A | C |
| ATOM | 2553 | CB | ASP | A | 576 | 2.750 | 5.995 | 0.225 | 1.00 | 38.18 | A | C |
| ATOM | 2556 | CG | ASP | A | 576 | 2.910 | 5.016 | 1.364 | 1.00 | 39.08 | A | C |
| ATOM | 2557 | OD1 | ASP | A | 576 | 2.937 | 3.796 | 1.076 | 1.00 | 38.78 | A | O |
| ATOM | 2558 | OD2 | ASP | A | 576 | 2.985 | 5.364 | 2.570 | 1.00 | 41.48 | A | O |
| ATOM | 2559 | C | ASP | A | 576 | 5.106 | 5.614 | −0.658 | 1.00 | 35.29 | A | C |
| ATOM | 2560 | O | ASP | A | 576 | 5.117 | 5.222 | −1.818 | 1.00 | 35.14 | A | O |
| ATOM | 2561 | N | GLU | A | 577 | 5.977 | 5.182 | 0.251 | 1.00 | 32.19 | A | N |
| ATOM | 2563 | CA | GLU | A | 577 | 7.146 | 4.377 | −0.133 | 1.00 | 29.90 | A | C |
| ATOM | 2565 | CB | GLU | A | 577 | 8.307 | 4.597 | 0.858 | 1.00 | 29.98 | A | C |
| ATOM | 2568 | CG | GLU | A | 577 | 8.924 | 5.992 | 0.745 | 1.00 | 29.86 | A | C |
| ATOM | 2571 | CD | GLU | A | 577 | 10.252 | 6.149 | 1.479 | 1.00 | 30.30 | A | C |
| ATOM | 2572 | OE1 | GLU | A | 577 | 11.230 | 5.443 | 1.141 | 1.00 | 27.60 | A | O |
| ATOM | 2573 | OE2 | GLU | A | 577 | 10.322 | 7.008 | 2.384 | 1.00 | 30.64 | A | O |
| ATOM | 2574 | C | GLU | A | 577 | 6.838 | 2.878 | −0.335 | 1.00 | 27.95 | A | C |
| ATOM | 2575 | O | GLU | A | 577 | 7.741 | 2.092 | −0.617 | 1.00 | 26.38 | A | O |
| ATOM | 2576 | N | ASP | A | 578 | 5.568 | 2.488 | −0.214 | 1.00 | 25.79 | A | N |
| ATOM | 2578 | CA | ASP | A | 578 | 5.152 | 1.130 | −0.565 | 1.00 | 24.73 | A | C |
| ATOM | 2580 | CB | ASP | A | 578 | 3.756 | 0.802 | −0.009 | 1.00 | 24.59 | A | C |
| ATOM | 2583 | CG | ASP | A | 578 | 3.755 | 0.507 | 1.479 | 1.00 | 24.22 | A | C |
| ATOM | 2584 | OD1 | ASP | A | 578 | 4.758 | 0.759 | 2.173 | 1.00 | 22.79 | A | O |
| ATOM | 2585 | OD2 | ASP | A | 578 | 2.761 | 0.021 | 2.040 | 1.00 | 23.67 | A | O |
| ATOM | 2586 | C | ASP | A | 578 | 5.113 | 0.914 | −2.085 | 1.00 | 24.30 | A | C |
| ATOM | 2587 | O | ASP | A | 578 | 5.010 | −0.223 | −2.538 | 1.00 | 22.85 | A | O |
| ATOM | 2588 | N | TYR | A | 579 | 5.172 | 2.001 | −2.859 | 1.00 | 24.45 | A | N |
| ATOM | 2590 | CA | TYR | A | 579 | 5.029 | 1.934 | −4.315 | 1.00 | 25.01 | A | C |
| ATOM | 2592 | CB | TYR | A | 579 | 3.928 | 2.908 | −4.770 | 1.00 | 25.04 | A | C |
| ATOM | 2595 | CG | TYR | A | 579 | 2.556 | 2.582 | −4.218 | 1.00 | 24.47 | A | C |
| ATOM | 2596 | CD1 | TYR | A | 579 | 2.195 | 2.963 | −2.928 | 1.00 | 25.69 | A | C |
| ATOM | 2598 | CE1 | TYR | A | 579 | 0.932 | 2.665 | −2.409 | 1.00 | 25.75 | A | C |
| ATOM | 2600 | CZ | TYR | A | 579 | 0.022 | 1.977 | −3.187 | 1.00 | 25.29 | A | C |
| ATOM | 2601 | OH | TYR | A | 579 | −1.217 | 1.675 | −2.675 | 1.00 | 25.28 | A | O |
| ATOM | 2603 | CE2 | TYR | A | 579 | 0.358 | 1.577 | −4.475 | 1.00 | 25.54 | A | C |
| ATOM | 2605 | CD2 | TYR | A | 579 | 1.620 | 1.886 | −4.984 | 1.00 | 26.01 | A | C |
| ATOM | 2607 | C | TYR | A | 579 | 6.329 | 2.209 | −5.093 | 1.00 | 25.81 | A | C |
| ATOM | 2608 | O | TYR | A | 579 | 6.401 | 1.925 | −6.285 | 1.00 | 26.17 | A | O |
| ATOM | 2609 | N | TYR | A | 580 | 7.349 | 2.751 | −4.435 | 1.00 | 26.41 | A | N |
| ATOM | 2611 | CA | TYR | A | 580 | 8.617 | 3.046 | −5.106 | 1.00 | 26.93 | A | C |
| ATOM | 2613 | CB | TYR | A | 580 | 8.598 | 4.475 | −5.677 | 1.00 | 27.09 | A | C |
| ATOM | 2616 | CG | TYR | A | 580 | 8.584 | 5.554 | −4.622 | 1.00 | 26.95 | A | C |
| ATOM | 2617 | CD1 | TYR | A | 580 | 9.771 | 6.087 | −4.123 | 1.00 | 27.01 | A | C |
| ATOM | 2619 | CE1 | TYR | A | 580 | 9.764 | 7.069 | −3.135 | 1.00 | 27.38 | A | C |
| ATOM | 2621 | CZ | TYR | A | 580 | 8.558 | 7.541 | −2.647 | 1.00 | 28.47 | A | C |
| ATOM | 2622 | OH | TYR | A | 580 | 8.545 | 8.516 | −1.674 | 1.00 | 29.60 | A | O |
| ATOM | 2624 | CE2 | TYR | A | 580 | 7.363 | 7.032 | −3.132 | 1.00 | 28.07 | A | C |
| ATOM | 2626 | CD2 | TYR | A | 580 | 7.381 | 6.043 | −4.113 | 1.00 | 27.45 | A | C |
| ATOM | 2628 | C | TYR | A | 580 | 9.823 | 2.867 | −4.183 | 1.00 | 27.08 | A | C |
| ATOM | 2629 | O | TYR | A | 580 | 9.705 | 2.962 | −2.949 | 1.00 | 27.01 | A | O |
| ATOM | 2630 | N | LYS | A | 581 | 10.981 | 2.607 | −4.787 | 1.00 | 27.09 | A | N |
| ATOM | 2632 | CA | LYS | A | 581 | 12.230 | 2.554 | −4.044 | 1.00 | 27.49 | A | C |
| ATOM | 2634 | CB | LYS | A | 581 | 13.167 | 1.469 | −4.591 | 1.00 | 27.82 | A | C |
| ATOM | 2637 | CG | LYS | A | 581 | 12.624 | 0.066 | −4.493 | 1.00 | 29.08 | A | C |
| ATOM | 2640 | CD | LYS | A | 581 | 12.553 | −0.410 | −3.054 | 1.00 | 29.95 | A | C |
| ATOM | 2643 | CE | LYS | A | 581 | 12.586 | −1.923 | −2.981 | 1.00 | 30.16 | A | C |
| ATOM | 2646 | NZ | LYS | A | 581 | 13.933 | −2.487 | −3.300 | 1.00 | 31.13 | A | N |
| ATOM | 2650 | C | LYS | A | 581 | 12.913 | 3.910 | −4.147 | 1.00 | 27.35 | A | C |
| ATOM | 2651 | O | LYS | A | 581 | 13.313 | 4.327 | −5.244 | 1.00 | 27.59 | A | O |
| ATOM | 2652 | N | ALA | A | 582 | 13.045 | 4.587 | −3.008 | 1.00 | 26.87 | A | N |
| ATOM | 2654 | CA | ALA | A | 582 | 13.721 | 5.877 | −2.940 | 1.00 | 27.05 | A | C |
| ATOM | 2656 | CB | ALA | A | 582 | 13.460 | 6.524 | −1.587 | 1.00 | 27.03 | A | C |
| ATOM | 2660 | C | ALA | A | 582 | 15.219 | 5.686 | −3.147 | 1.00 | 27.17 | A | C |
| ATOM | 2661 | O | ALA | A | 582 | 15.770 | 4.669 | −2.733 | 1.00 | 26.90 | A | O |
| ATOM | 2662 | N | SER | A | 583 | 15.875 | 6.657 | −3.789 | 1.00 | 26.98 | A | N |
| ATOM | 2664 | CA | SER | A | 583 | 17.342 | 6.691 | −3.818 | 1.00 | 27.27 | A | C |
| ATOM | 2666 | CB | SER | A | 583 | 17.864 | 7.934 | −4.568 | 1.00 | 27.02 | A | C |
| ATOM | 2669 | OG | SER | A | 583 | 17.666 | 7.842 | −5.971 | 1.00 | 24.95 | A | O |
| ATOM | 2671 | C | SER | A | 583 | 17.899 | 6.673 | −2.390 | 1.00 | 27.51 | A | C |
| ATOM | 2672 | O | SER | A | 583 | 18.853 | 5.960 | −2.111 | 1.00 | 28.19 | A | O |
| ATOM | 2673 | N | VAL | A | 584 | 17.301 | 7.481 | −1.511 | 1.00 | 27.95 | A | N |
| ATOM | 2675 | CA | VAL | A | 584 | 17.624 | 7.547 | −0.081 | 1.00 | 27.95 | A | C |
| ATOM | 2677 | CB | VAL | A | 584 | 18.359 | 8.860 | 0.279 | 1.00 | 28.18 | A | C |
| ATOM | 2679 | CG1 | VAL | A | 584 | 18.684 | 8.910 | 1.762 | 1.00 | 29.19 | A | C |
| ATOM | 2683 | CG2 | VAL | A | 584 | 19.610 | 8.997 | −0.529 | 1.00 | 28.53 | A | C |
| ATOM | 2687 | C | VAL | A | 584 | 16.314 | 7.499 | 0.716 | 1.00 | 27.93 | A | C |
| ATOM | 2688 | O | VAL | A | 584 | 15.532 | 8.445 | 0.693 | 1.00 | 27.03 | A | O |
| ATOM | 2689 | N | THR | A | 585 | 16.072 | 6.403 | 1.428 | 1.00 | 28.20 | A | N |
| ATOM | 2691 | CA | THR | A | 585 | 14.795 | 6.239 | 2.111 | 1.00 | 28.72 | A | C |
| ATOM | 2693 | CB | THR | A | 585 | 14.394 | 4.744 | 2.181 | 1.00 | 28.79 | A | C |
| ATOM | 2695 | OG1 | THR | A | 585 | 13.033 | 4.623 | 2.621 | 1.00 | 29.29 | A | O |
| ATOM | 2697 | CG2 | THR | A | 585 | 15.193 | 3.992 | 3.216 | 1.00 | 29.13 | A | C |
| ATOM | 2701 | C | THR | A | 585 | 14.779 | 6.882 | 3.494 | 1.00 | 28.58 | A | C |
| ATOM | 2702 | O | THR | A | 585 | 15.809 | 7.081 | 4.129 | 1.00 | 29.24 | A | O |
| ATOM | 2703 | N | ARG | A | 586 | 13.572 | 7.185 | 3.951 | 1.00 | 28.53 | A | N |
| ATOM | 2705 | CA | ARG | A | 586 | 13.331 | 7.764 | 5.266 | 1.00 | 27.88 | A | C |
| ATOM | 2707 | CB | ARG | A | 586 | 12.163 | 8.751 | 5.159 | 1.00 | 28.97 | A | C |
| ATOM | 2710 | CG | ARG | A | 586 | 12.163 | 9.613 | 3.892 | 1.00 | 32.87 | A | C |
| ATOM | 2713 | CD | ARG | A | 586 | 11.091 | 10.706 | 3.880 | 1.00 | 37.35 | A | C |
| ATOM | 2716 | NE | ARG | A | 586 | 11.446 | 11.869 | 4.700 | 1.00 | 40.84 | A | N |
| ATOM | 2718 | CZ | ARG | A | 586 | 12.287 | 12.850 | 4.340 | 1.00 | 42.20 | A | C |
| ATOM | 2719 | NH1 | ARG | A | 586 | 12.897 | 12.839 | 3.157 | 1.00 | 42.91 | A | N |
| ATOM | 2722 | NH2 | ARG | A | 586 | 12.524 | 13.854 | 5.183 | 1.00 | 42.67 | A | N |
| ATOM | 2725 | C | ARG | A | 586 | 12.978 | 6.639 | 6.271 | 1.00 | 25.82 | A | C |
| ATOM | 2726 | O | ARG | A | 586 | 12.956 | 6.850 | 7.485 | 1.00 | 25.52 | A | O |
| ATOM | 2727 | N | LEU | A | 587 | 12.724 | 5.449 | 5.742 | 1.00 | 23.19 | A | N |
| ATOM | 2729 | CA | LEU | A | 587 | 12.216 | 4.320 | 6.520 | 1.00 | 21.02 | A | C |
| ATOM | 2731 | CB | LEU | A | 587 | 11.740 | 3.228 | 5.569 | 1.00 | 21.26 | A | C |
| ATOM | 2734 | CG | LEU | A | 587 | 10.591 | 3.508 | 4.618 | 1.00 | 22.52 | A | C |
| ATOM | 2736 | CD1 | LEU | A | 587 | 10.428 | 2.299 | 3.713 | 1.00 | 23.24 | A | C |
| ATOM | 2740 | CD2 | LEU | A | 587 | 9.314 | 3.812 | 5.391 | 1.00 | 23.89 | A | C |
| ATOM | 2744 | C | LEU | A | 587 | 13.303 | 3.726 | 7.410 | 1.00 | 19.25 | A | C |
| ATOM | 2745 | O | LEU | A | 587 | 14.475 | 3.822 | 7.075 | 1.00 | 18.79 | A | O |
| ATOM | 2746 | N | PRO | A | 588 | 12.911 | 3.068 | 8.506 | 1.00 | 16.35 | A | N |
| ATOM | 2747 | CA | PRO | A | 588 | 13.877 | 2.494 | 9.451 | 1.00 | 14.93 | A | C |
| ATOM | 2749 | CB | PRO | A | 588 | 13.037 | 2.286 | 10.717 | 1.00 | 14.98 | A | C |
| ATOM | 2752 | CG | PRO | A | 588 | 11.683 | 1.973 | 10.177 | 1.00 | 15.18 | A | C |
| ATOM | 2755 | CD | PRO | A | 588 | 11.513 | 2.828 | 8.927 | 1.00 | 16.26 | A | C |
| ATOM | 2758 | C | PRO | A | 588 | 14.465 | 1.175 | 8.947 | 1.00 | 13.29 | A | C |
| ATOM | 2759 | O | PRO | A | 588 | 14.183 | 0.083 | 9.484 | 1.00 | 12.58 | A | O |
| ATOM | 2760 | N | ILE | A | 589 | 15.309 | 1.281 | 7.925 | 1.00 | 11.92 | A | N |
| ATOM | 2762 | CA | ILE | A | 589 | 15.866 | 0.118 | 7.230 | 1.00 | 11.54 | A | C |
| ATOM | 2764 | CB | ILE | A | 589 | 16.889 | 0.600 | 6.152 | 1.00 | 11.43 | A | C |
| ATOM | 2766 | CG1 | ILE | A | 589 | 16.174 | 1.366 | 5.024 | 1.00 | 13.60 | A | C |
| ATOM | 2769 | CD1 | ILE | A | 589 | 15.092 | 0.544 | 4.305 | 1.00 | 14.47 | A | C |
| ATOM | 2773 | CG2 | ILE | A | 589 | 17.700 | −0.581 | 5.601 | 1.00 | 12.99 | A | C |
| ATOM | 2777 | C | ILE | A | 589 | 16.574 | −0.864 | 8.203 | 1.00 | 10.07 | A | C |
| ATOM | 2778 | O | ILE | A | 589 | 16.471 | −2.075 | 8.055 | 1.00 | 9.44 | A | O |
| ATOM | 2779 | N | LYS | A | 590 | 17.300 | −0.330 | 9.186 | 1.00 | 9.76 | A | N |
| ATOM | 2781 | CA | LYS | A | 590 | 18.065 | −1.186 | 10.109 | 1.00 | 9.66 | A | C |
| ATOM | 2783 | CB | LYS | A | 590 | 19.111 | −0.377 | 10.888 | 1.00 | 9.94 | A | C |
| ATOM | 2786 | CG | LYS | A | 590 | 20.252 | 0.122 | 10.016 | 1.00 | 10.30 | A | C |
| ATOM | 2789 | CD | LYS | A | 590 | 21.098 | 1.207 | 10.703 | 1.00 | 10.44 | A | C |
| ATOM | 2792 | CE | LYS | A | 590 | 22.248 | 1.579 | 9.797 | 1.00 | 12.41 | A | C |
| ATOM | 2795 | NZ | LYS | A | 590 | 23.160 | 2.618 | 10.380 | 1.00 | 12.07 | A | N |
| ATOM | 2799 | C | LYS | A | 590 | 17.174 | −1.954 | 11.068 | 1.00 | 9.73 | A | C |
| ATOM | 2800 | O | LYS | A | 590 | 17.660 | −2.830 | 11.790 | 1.00 | 9.50 | A | O |
| ATOM | 2801 | N | TRP | A | 591 | 15.876 | −1.628 | 11.089 | 1.00 | 9.17 | A | N |
| ATOM | 2803 | CA | TRP | A | 591 | 14.905 | −2.339 | 11.912 | 1.00 | 9.87 | A | C |
| ATOM | 2805 | CB | TRP | A | 591 | 14.017 | −1.349 | 12.662 | 1.00 | 9.98 | A | C |
| ATOM | 2808 | CG | TRP | A | 591 | 14.655 | −0.542 | 13.766 | 1.00 | 11.71 | A | C |
| ATOM | 2809 | CD1 | TRP | A | 591 | 14.457 | −0.709 | 15.103 | 1.00 | 12.20 | A | C |
| ATOM | 2811 | NE1 | TRP | A | 591 | 15.141 | 0.252 | 15.803 | 1.00 | 13.37 | A | N |
| ATOM | 2813 | CE2 | TRP | A | 591 | 15.793 | 1.065 | 14.924 | 1.00 | 12.15 | A | C |
| ATOM | 2814 | CD2 | TRP | A | 591 | 15.490 | 0.607 | 13.627 | 1.00 | 13.10 | A | C |
| ATOM | 2815 | CE3 | TRP | A | 591 | 16.031 | 1.299 | 12.533 | 1.00 | 12.17 | A | C |
| ATOM | 2817 | CZ3 | TRP | A | 591 | 16.853 | 2.386 | 12.772 | 1.00 | 15.31 | A | C |
| ATOM | 2819 | CH2 | TRP | A | 591 | 17.127 | 2.813 | 14.082 | 1.00 | 13.32 | A | C |
| ATOM | 2821 | CZ2 | TRP | A | 591 | 16.621 | 2.156 | 15.161 | 1.00 | 12.48 | A | C |
| ATOM | 2823 | C | TRP | A | 591 | 13.997 | −3.282 | 11.108 | 1.00 | 9.92 | A | C |
| ATOM | 2824 | O | TRP | A | 591 | 13.223 | −4.039 | 11.706 | 1.00 | 10.09 | A | O |
| ATOM | 2825 | N | MET | A | 592 | 14.096 | −3.251 | 9.773 | 1.00 | 9.75 | A | N |
| ATOM | 2827 | CA | MET | A | 592 | 13.094 | −3.843 | 8.881 | 1.00 | 9.99 | A | C |
| ATOM | 2829 | CB | MET | A | 592 | 12.876 | −2.908 | 7.667 | 1.00 | 9.83 | A | C |
| ATOM | 2832 | CG | MET | A | 592 | 12.051 | −1.676 | 7.999 | 1.00 | 11.13 | A | C |
| ATOM | 2835 | SD | MET | A | 592 | 12.111 | −0.339 | 6.763 | 1.00 | 14.42 | A | S |
| ATOM | 2836 | CE | MET | A | 592 | 11.786 | −1.271 | 5.270 | 1.00 | 11.86 | A | C |
| ATOM | 2840 | C | MET | A | 592 | 13.432 | −5.244 | 8.373 | 1.00 | 9.53 | A | C |
| ATOM | 2841 | O | MET | A | 592 | 14.602 | −5.589 | 8.192 | 1.00 | 10.45 | A | O |
| ATOM | 2842 | N | SER | A | 593 | 12.395 | −6.034 | 8.118 | 1.00 | 9.01 | A | N |
| ATOM | 2844 | CA | SER | A | 593 | 12.558 | −7.391 | 7.605 | 1.00 | 9.63 | A | C |
| ATOM | 2846 | CB | SER | A | 593 | 11.241 | −8.149 | 7.607 | 1.00 | 10.07 | A | C |
| ATOM | 2849 | OG | SER | A | 593 | 10.406 | −7.605 | 6.617 | 1.00 | 10.36 | A | O |
| ATOM | 2851 | C | SER | A | 593 | 13.073 | −7.313 | 6.164 | 1.00 | 9.56 | A | C |
| ATOM | 2852 | O | SER | A | 593 | 12.921 | −6.287 | 5.511 | 1.00 | 10.05 | A | O |
| ATOM | 2853 | N | PRO | A | 594 | 13.695 | −8.378 | 5.686 | 1.00 | 10.88 | A | N |
| ATOM | 2854 | CA | PRO | A | 594 | 14.197 | −8.420 | 4.301 | 1.00 | 11.51 | A | C |
| ATOM | 2856 | CB | PRO | A | 594 | 14.787 | −9.828 | 4.191 | 1.00 | 12.13 | A | C |
| ATOM | 2859 | CG | PRO | A | 594 | 15.141 | −10.176 | 5.577 | 1.00 | 12.75 | A | C |
| ATOM | 2862 | CD | PRO | A | 594 | 14.022 | −9.610 | 6.420 | 1.00 | 10.32 | A | C |
| ATOM | 2865 | C | PRO | A | 594 | 13.108 | −8.199 | 3.254 | 1.00 | 11.67 | A | C |
| ATOM | 2866 | O | PRO | A | 594 | 13.366 | −7.517 | 2.289 | 1.00 | 12.87 | A | O |
| ATOM | 2867 | N | GLU | A | 595 | 11.916 | −8.754 | 3.455 | 1.00 | 12.18 | A | N |
| ATOM | 2869 | CA | GLU | A | 595 | 10.819 | −8.588 | 2.509 | 1.00 | 11.54 | A | C |
| ATOM | 2871 | CB | GLU | A | 595 | 9.685 | −9.578 | 2.796 | 1.00 | 11.95 | A | C |
| ATOM | 2874 | CG | GLU | A | 595 | 8.905 | −9.349 | 4.080 | 1.00 | 12.54 | A | C |
| ATOM | 2877 | CD | GLU | A | 595 | 9.410 | −10.169 | 5.261 | 1.00 | 12.35 | A | C |
| ATOM | 2878 | OE1 | GLU | A | 595 | 10.587 | −10.628 | 5.259 | 1.00 | 12.67 | A | O |
| ATOM | 2879 | OE2 | GLU | A | 595 | 8.624 | −10.313 | 6.232 | 1.00 | 11.80 | A | O |
| ATOM | 2880 | C | GLU | A | 595 | 10.325 | −7.139 | 2.494 | 1.00 | 11.35 | A | C |
| ATOM | 2881 | O | GLU | A | 595 | 9.870 | −6.621 | 1.462 | 1.00 | 10.36 | A | O |
| ATOM | 2882 | N | SER | A | 596 | 10.453 | −6.468 | 3.639 | 1.00 | 11.09 | A | N |
| ATOM | 2884 | CA | SER | A | 596 | 10.129 | −5.054 | 3.746 | 1.00 | 11.11 | A | C |
| ATOM | 2886 | CB | SER | A | 596 | 10.035 | −4.639 | 5.215 | 1.00 | 11.61 | A | C |
| ATOM | 2889 | OG | SER | A | 596 | 9.071 | −5.412 | 5.916 | 1.00 | 10.96 | A | O |
| ATOM | 2891 | C | SER | A | 596 | 11.154 | −4.165 | 3.009 | 1.00 | 11.46 | A | C |
| ATOM | 2892 | O | SER | A | 596 | 10.780 | −3.187 | 2.349 | 1.00 | 11.08 | A | O |
| ATOM | 2893 | N | ILE | A | 597 | 12.433 | −4.494 | 3.154 | 1.00 | 11.82 | A | N |
| ATOM | 2895 | CA | ILE | A | 597 | 13.498 | −3.793 | 2.447 | 1.00 | 12.20 | A | C |
| ATOM | 2897 | CB | ILE | A | 597 | 14.870 | −4.196 | 3.012 | 1.00 | 12.29 | A | C |
| ATOM | 2899 | CG1 | ILE | A | 597 | 15.044 | −3.734 | 4.479 | 1.00 | 11.10 | A | C |
| ATOM | 2902 | CD1 | ILE | A | 597 | 16.321 | −4.267 | 5.140 | 1.00 | 10.75 | A | C |
| ATOM | 2906 | CG2 | ILE | A | 597 | 15.980 | −3.586 | 2.160 | 1.00 | 13.21 | A | C |
| ATOM | 2910 | C | ILE | A | 597 | 13.433 | −4.051 | 0.923 | 1.00 | 12.61 | A | C |
| ATOM | 2911 | O | ILE | A | 597 | 13.521 | −3.120 | 0.135 | 1.00 | 14.25 | A | O |
| ATOM | 2912 | N | ASN | A | 598 | 13.275 | −5.298 | 0.514 | 1.00 | 12.98 | A | N |
| ATOM | 2914 | CA | ASN | A | 598 | 13.331 | −5.672 | −0.919 | 1.00 | 14.27 | A | C |
| ATOM | 2916 | CB | ASN | A | 598 | 13.618 | −7.179 | −1.052 | 1.00 | 14.04 | A | C |
| ATOM | 2919 | CG | ASN | A | 598 | 15.068 | −7.523 | −0.790 | 1.00 | 16.01 | A | C |
| ATOM | 2920 | OD1 | ASN | A | 598 | 15.971 | −6.727 | −1.066 | 1.00 | 18.13 | A | O |
| ATOM | 2921 | ND2 | ASN | A | 598 | 15.307 | −8.734 | −0.283 | 1.00 | 16.92 | A | N |
| ATOM | 2924 | C | ASN | A | 598 | 12.077 | −5.337 | −1.725 | 1.00 | 15.32 | A | C |
| ATOM | 2925 | O | ASN | A | 598 | 12.162 | −4.886 | −2.877 | 1.00 | 15.17 | A | O |
| ATOM | 2926 | N | PHE | A | 599 | 10.912 | −5.552 | −1.119 | 1.00 | 15.42 | A | N |
| ATOM | 2928 | CA | PHE | A | 599 | 9.636 | −5.496 | −1.839 | 1.00 | 15.79 | A | C |
| ATOM | 2930 | CB | PHE | A | 599 | 9.088 | −6.914 | −2.039 | 1.00 | 15.78 | A | C |
| ATOM | 2933 | CG | PHE | A | 599 | 10.095 | −7.884 | −2.566 | 1.00 | 18.17 | A | C |
| ATOM | 2934 | CD1 | PHE | A | 599 | 10.746 | −7.643 | −3.769 | 1.00 | 20.18 | A | C |
| ATOM | 2936 | CE1 | PHE | A | 599 | 11.694 | −8.537 | −4.245 | 1.00 | 21.90 | A | C |
| ATOM | 2938 | CZ | PHE | A | 599 | 11.975 | −9.693 | −3.532 | 1.00 | 20.74 | A | C |
| ATOM | 2940 | CE2 | PHE | A | 599 | 11.318 | −9.945 | −2.336 | 1.00 | 21.69 | A | C |
| ATOM | 2942 | CD2 | PHE | A | 599 | 10.391 | −9.038 | −1.860 | 1.00 | 19.49 | A | C |
| ATOM | 2944 | C | PHE | A | 599 | 8.574 | −4.641 | −1.156 | 1.00 | 15.14 | A | C |
| ATOM | 2945 | O | PHE | A | 599 | 7.427 | −4.610 | −1.608 | 1.00 | 15.42 | A | O |
| ATOM | 2946 | N | ARG | A | 600 | 8.951 | −3.936 | −0.092 | 1.00 | 15.13 | A | N |
| ATOM | 2948 | CA | ARG | A | 600 | 8.024 | −3.137 | 0.701 | 1.00 | 15.85 | A | C |
| ATOM | 2950 | CB | ARG | A | 600 | 7.614 | −1.880 | −0.076 | 1.00 | 16.31 | A | C |
| ATOM | 2953 | CG | ARG | A | 600 | 8.784 | −0.950 | −0.377 | 1.00 | 17.77 | A | C |
| ATOM | 2956 | CD | ARG | A | 600 | 9.269 | −0.173 | 0.835 | 1.00 | 18.17 | A | C |
| ATOM | 2959 | NE | ARG | A | 600 | 10.355 | 0.750 | 0.508 | 1.00 | 20.72 | A | N |
| ATOM | 2961 | CZ | ARG | A | 600 | 11.657 | 0.490 | 0.633 | 1.00 | 21.90 | A | C |
| ATOM | 2962 | NH1 | ARG | A | 600 | 12.094 | −0.680 | 1.078 | 1.00 | 22.93 | A | N |
| ATOM | 2965 | NH2 | ARG | A | 600 | 12.543 | 1.419 | 0.299 | 1.00 | 22.61 | A | N |
| ATOM | 2968 | C | ARG | A | 600 | 6.793 | −3.956 | 1.149 | 1.00 | 15.58 | A | C |
| ATOM | 2969 | O | ARG | A | 600 | 5.653 | −3.464 | 1.136 | 1.00 | 15.40 | A | O |
| ATOM | 2970 | N | ARG | A | 601 | 7.046 | −5.206 | 1.535 | 1.00 | 15.64 | A | N |
| ATOM | 2972 | CA | ARG | A | 601 | 6.022 | −6.120 | 2.052 | 1.00 | 16.29 | A | C |
| ATOM | 2974 | CB | ARG | A | 601 | 6.363 | −7.569 | 1.724 | 1.00 | 17.23 | A | C |
| ATOM | 2977 | CG | ARG | A | 601 | 6.233 | −7.938 | 0.259 | 1.00 | 21.68 | A | C |
| ATOM | 2980 | CD | ARG | A | 601 | 5.955 | −9.440 | −0.022 | 1.00 | 25.56 | A | C |
| ATOM | 2983 | NE | ARG | A | 601 | 6.621 | −9.837 | −1.270 | 1.00 | 30.36 | A | N |
| ATOM | 2985 | CZ | ARG | A | 601 | 6.043 | −10.295 | −2.379 | 1.00 | 31.71 | A | C |
| ATOM | 2986 | NH1 | ARG | A | 601 | 4.738 | −10.497 | −2.462 | 1.00 | 34.89 | A | N |
| ATOM | 2989 | NH2 | ARG | A | 601 | 6.800 | −10.599 | −3.428 | 1.00 | 34.61 | A | N |
| ATOM | 2992 | C | ARG | A | 601 | 6.002 | −5.963 | 3.571 | 1.00 | 15.73 | A | C |
| ATOM | 2993 | O | ARG | A | 601 | 7.033 | −6.218 | 4.233 | 1.00 | 15.42 | A | O |
| ATOM | 2994 | N | PHE | A | 602 | 4.866 | −5.511 | 4.105 | 1.00 | 14.28 | A | N |
| ATOM | 2996 | CA | PHE | A | 602 | 4.684 | −5.323 | 5.543 | 1.00 | 14.03 | A | C |
| ATOM | 2998 | CB | PHE | A | 602 | 4.463 | −3.844 | 5.876 | 1.00 | 13.94 | A | C |
| ATOM | 3001 | CG | PHE | A | 602 | 5.599 | −2.947 | 5.501 | 1.00 | 15.36 | A | C |
| ATOM | 3002 | CD1 | PHE | A | 602 | 6.596 | −2.632 | 6.430 | 1.00 | 16.03 | A | C |
| ATOM | 3004 | CE1 | PHE | A | 602 | 7.651 | −1.772 | 6.090 | 1.00 | 15.79 | A | C |
| ATOM | 3006 | CZ | PHE | A | 602 | 7.690 | −1.198 | 4.833 | 1.00 | 15.40 | A | C |
| ATOM | 3008 | CE2 | PHE | A | 602 | 6.686 | −1.485 | 3.909 | 1.00 | 15.83 | A | C |
| ATOM | 3010 | CD2 | PHE | A | 602 | 5.643 | −2.348 | 4.245 | 1.00 | 14.53 | A | C |
| ATOM | 3012 | C | PHE | A | 602 | 3.499 | −6.130 | 6.054 | 1.00 | 13.39 | A | C |
| ATOM | 3013 | O | PHE | A | 602 | 2.353 | −5.927 | 5.631 | 1.00 | 13.20 | A | O |
| ATOM | 3014 | N | THR | A | 603 | 3.766 | −7.070 | 6.947 | 1.00 | 13.12 | A | N |
| ATOM | 3016 | CA | THR | A | 603 | 2.739 | −7.941 | 7.518 | 1.00 | 12.82 | A | C |
| ATOM | 3018 | CB | THR | A | 603 | 2.851 | −9.331 | 6.902 | 1.00 | 13.20 | A | C |
| ATOM | 3020 | OG1 | THR | A | 603 | 4.143 | −9.881 | 7.212 | 1.00 | 13.90 | A | O |
| ATOM | 3022 | CG2 | THR | A | 603 | 2.771 | −9.277 | 5.357 | 1.00 | 14.67 | A | C |
| ATOM | 3026 | C | THR | A | 603 | 2.968 | −8.095 | 9.007 | 1.00 | 12.15 | A | C |
| ATOM | 3027 | O | THR | A | 603 | 3.936 | −7.588 | 9.552 | 1.00 | 10.36 | A | O |
| ATOM | 3028 | N | THR | A | 604 | 2.125 | −8.874 | 9.659 | 1.00 | 11.47 | A | N |
| ATOM | 3030 | CA | THR | A | 604 | 2.387 | −9.169 | 11.053 | 1.00 | 11.50 | A | C |
| ATOM | 3032 | CB | THR | A | 604 | 1.228 | −9.955 | 11.676 | 1.00 | 12.26 | A | C |
| ATOM | 3034 | OG1 | THR | A | 604 | 0.043 | −9.138 | 11.648 | 1.00 | 14.28 | A | O |
| ATOM | 3036 | CG2 | THR | A | 604 | 1.493 | −10.182 | 13.155 | 1.00 | 14.14 | A | C |
| ATOM | 3040 | C | THR | A | 604 | 3.728 | −9.884 | 11.203 | 1.00 | 11.13 | A | C |
| ATOM | 3041 | O | THR | A | 604 | 4.391 | −9.699 | 12.211 | 1.00 | 9.85 | A | O |
| ATOM | 3042 | N | ALA | A | 605 | 4.142 | −10.679 | 10.206 | 1.00 | 10.23 | A | N |
| ATOM | 3044 | CA | ALA | A | 605 | 5.450 | −11.338 | 10.258 | 1.00 | 10.32 | A | C |
| ATOM | 3046 | CB | ALA | A | 605 | 5.608 | −12.374 | 9.156 | 1.00 | 10.78 | A | C |
| ATOM | 3050 | C | ALA | A | 605 | 6.611 | −10.363 | 10.197 | 1.00 | 9.56 | A | C |
| ATOM | 3051 | O | ALA | A | 605 | 7.640 | −10.616 | 10.824 | 1.00 | 8.50 | A | O |
| ATOM | 3052 | N | SER | A | 606 | 6.459 | −9.272 | 9.445 | 1.00 | 8.64 | A | N |
| ATOM | 3054 | CA | SER | A | 606 | 7.486 | −8.245 | 9.412 | 1.00 | 9.24 | A | C |
| ATOM | 3056 | CB | SER | A | 606 | 7.352 | −7.296 | 8.190 | 1.00 | 9.76 | A | C |
| ATOM | 3059 | OG | SER | A | 606 | 6.179 | −6.496 | 8.250 | 1.00 | 10.66 | A | O |
| ATOM | 3061 | C | SER | A | 606 | 7.505 | −7.489 | 10.748 | 1.00 | 9.16 | A | C |
| ATOM | 3062 | O | SER | A | 606 | 8.572 | −7.100 | 11.211 | 1.00 | 8.11 | A | O |
| ATOM | 3063 | N | ASP | A | 607 | 6.340 | −7.307 | 11.379 | 1.00 | 8.37 | A | N |
| ATOM | 3065 | CA | ASP | A | 607 | 6.297 | −6.717 | 12.720 | 1.00 | 8.68 | A | C |
| ATOM | 3067 | CB | ASP | A | 607 | 4.876 | −6.507 | 13.225 | 1.00 | 8.94 | A | C |
| ATOM | 3070 | CG | ASP | A | 607 | 4.256 | −5.197 | 12.785 | 1.00 | 11.42 | A | C |
| ATOM | 3071 | OD1 | ASP | A | 607 | 4.925 | −4.280 | 12.218 | 1.00 | 11.99 | A | O |
| ATOM | 3072 | OD2 | ASP | A | 607 | 3.029 | −5.001 | 13.022 | 1.00 | 12.74 | A | O |
| ATOM | 3073 | C | ASP | A | 607 | 7.034 | −7.616 | 13.740 | 1.00 | 8.12 | A | C |
| ATOM | 3074 | O | ASP | A | 607 | 7.628 | −7.111 | 14.678 | 1.00 | 7.11 | A | O |
| ATOM | 3075 | N | VAL | A | 608 | 6.952 | −8.940 | 13.577 | 1.00 | 7.25 | A | N |
| ATOM | 3077 | CA | VAL | A | 608 | 7.677 | −9.870 | 14.461 | 1.00 | 7.14 | A | C |
| ATOM | 3079 | CB | VAL | A | 608 | 7.280 | −11.342 | 14.171 | 1.00 | 6.69 | A | C |
| ATOM | 3081 | CG1 | VAL | A | 608 | 8.277 | −12.331 | 14.822 | 1.00 | 8.13 | A | C |
| ATOM | 3085 | CG2 | VAL | A | 608 | 5.840 | −11.616 | 14.668 | 1.00 | 7.95 | A | C |
| ATOM | 3089 | C | VAL | A | 608 | 9.187 | −9.687 | 14.332 | 1.00 | 7.61 | A | C |
| ATOM | 3090 | O | VAL | A | 608 | 9.902 | −9.615 | 15.345 | 1.00 | 6.65 | A | O |
| ATOM | 3091 | N | TRP | A | 609 | 9.673 | −9.587 | 13.090 | 1.00 | 7.07 | A | N |
| ATOM | 3093 | CA | TRP | A | 609 | 11.085 | −9.274 | 12.835 | 1.00 | 7.28 | A | C |
| ATOM | 3095 | CB | TRP | A | 609 | 11.349 | −9.095 | 11.329 | 1.00 | 7.08 | A | C |
| ATOM | 3098 | CG | TRP | A | 609 | 12.771 | −8.707 | 10.985 | 1.00 | 7.95 | A | C |
| ATOM | 3099 | CD1 | TRP | A | 609 | 13.381 | −7.506 | 11.228 | 1.00 | 7.59 | A | C |
| ATOM | 3101 | NE1 | TRP | A | 609 | 14.692 | −7.555 | 10.821 | 1.00 | 8.83 | A | N |
| ATOM | 3103 | CE2 | TRP | A | 609 | 14.958 | −8.799 | 10.310 | 1.00 | 8.13 | A | C |
| ATOM | 3104 | CD2 | TRP | A | 609 | 13.778 | −9.555 | 10.414 | 1.00 | 6.30 | A | C |
| ATOM | 3105 | CE3 | TRP | A | 609 | 13.779 | −10.881 | 9.934 | 1.00 | 6.84 | A | C |
| ATOM | 3107 | CZ3 | TRP | A | 609 | 14.944 | −11.401 | 9.398 | 1.00 | 8.38 | A | C |
| ATOM | 3109 | CH2 | TRP | A | 609 | 16.108 | −10.624 | 9.313 | 1.00 | 7.22 | A | C |
| ATOM | 3111 | CZ2 | TRP | A | 609 | 16.135 | −9.313 | 9.749 | 1.00 | 8.92 | A | C |
| ATOM | 3113 | C | TRP | A | 609 | 11.484 | −7.994 | 13.602 | 1.00 | 7.00 | A | C |
| ATOM | 3114 | O | TRP | A | 609 | 12.484 | −7.969 | 14.329 | 1.00 | 7.89 | A | O |
| ATOM | 3115 | N | MET | A | 610 | 10.687 | −6.954 | 13.449 | 1.00 | 7.31 | A | N |
| ATOM | 3117 | CA | MET | A | 610 | 11.019 | −5.634 | 13.979 | 1.00 | 7.12 | A | C |
| ATOM | 3119 | CB | MET | A | 610 | 10.083 | −4.569 | 13.380 | 1.00 | 7.25 | A | C |
| ATOM | 3122 | CG | MET | A | 610 | 10.433 | −3.155 | 13.777 | 1.00 | 8.77 | A | C |
| ATOM | 3125 | SD | MET | A | 610 | 9.336 | −1.944 | 13.037 | 1.00 | 11.18 | A | S |
| ATOM | 3126 | CE | MET | A | 610 | 10.448 | −0.489 | 13.042 | 1.00 | 12.69 | A | C |
| ATOM | 3130 | C | MET | A | 610 | 10.945 | −5.628 | 15.504 | 1.00 | 7.16 | A | C |
| ATOM | 3131 | O | MET | A | 610 | 11.740 | −4.979 | 16.171 | 1.00 | 7.31 | A | O |
| ATOM | 3132 | N | PHE | A | 611 | 9.995 | −6.376 | 16.053 | 1.00 | 7.20 | A | N |
| ATOM | 3134 | CA | PHE | A | 611 | 9.859 | −6.506 | 17.503 | 1.00 | 6.98 | A | C |
| ATOM | 3136 | CB | PHE | A | 611 | 8.614 | −7.289 | 17.857 | 1.00 | 7.02 | A | C |
| ATOM | 3139 | CG | PHE | A | 611 | 8.576 | −7.724 | 19.296 | 1.00 | 8.33 | A | C |
| ATOM | 3140 | CD1 | PHE | A | 611 | 8.329 | −6.808 | 20.294 | 1.00 | 9.98 | A | C |
| ATOM | 3142 | CE1 | PHE | A | 611 | 8.322 | −7.188 | 21.610 | 1.00 | 9.82 | A | C |
| ATOM | 3144 | CZ | PHE | A | 611 | 8.587 | −8.482 | 21.958 | 1.00 | 9.72 | A | C |
| ATOM | 3146 | CE2 | PHE | A | 611 | 8.835 | −9.415 | 20.984 | 1.00 | 11.54 | A | C |
| ATOM | 3148 | CD2 | PHE | A | 611 | 8.838 | −9.033 | 19.645 | 1.00 | 9.39 | A | C |
| ATOM | 3150 | C | PHE | A | 611 | 11.103 | −7.142 | 18.153 | 1.00 | 6.73 | A | C |
| ATOM | 3151 | O | PHE | A | 611 | 11.557 | −6.708 | 19.202 | 1.00 | 7.59 | A | O |
| ATOM | 3152 | N | ALA | A | 612 | 11.692 | −8.119 | 17.491 | 1.00 | 7.27 | A | N |
| ATOM | 3154 | CA | ALA | A | 612 | 12.922 | −8.703 | 17.973 | 1.00 | 7.36 | A | C |
| ATOM | 3156 | CB | ALA | A | 612 | 13.212 | −9.987 | 17.272 | 1.00 | 7.52 | A | C |
| ATOM | 3160 | C | ALA | A | 612 | 14.090 | −7.718 | 17.877 | 1.00 | 7.40 | A | C |
| ATOM | 3161 | O | ALA | A | 612 | 14.984 | −7.738 | 18.735 | 1.00 | 7.06 | A | O |
| ATOM | 3162 | N | VAL | A | 613 | 14.094 | −6.842 | 16.868 | 1.00 | 7.17 | A | N |
| ATOM | 3164 | CA | VAL | A | 613 | 15.098 | −5.779 | 16.831 | 1.00 | 7.65 | A | C |
| ATOM | 3166 | CB | VAL | A | 613 | 15.096 | −4.939 | 15.516 | 1.00 | 7.18 | A | C |
| ATOM | 3168 | CG1 | VAL | A | 613 | 16.183 | −3.867 | 15.575 | 1.00 | 8.13 | A | C |
| ATOM | 3172 | CG2 | VAL | A | 613 | 15.284 | −5.822 | 14.301 | 1.00 | 8.64 | A | C |
| ATOM | 3176 | C | VAL | A | 613 | 14.874 | −4.843 | 18.018 | 1.00 | 7.44 | A | C |
| ATOM | 3177 | O | VAL | A | 613 | 15.816 | −4.438 | 18.663 | 1.00 | 7.86 | A | O |
| ATOM | 3178 | N | CYS | A | 614 | 13.628 | −4.521 | 18.312 | 1.00 | 7.31 | A | N |
| ATOM | 3180 | CA | CYS | A | 614 | 13.304 | −3.712 | 19.482 | 1.00 | 7.38 | A | C |
| ATOM | 3182 | CB | CYS | A | 614 | 11.808 | −3.409 | 19.500 | 1.00 | 7.62 | A | C |
| ATOM | 3185 | SG | CYS | A | 614 | 11.279 | −2.363 | 20.856 | 1.00 | 12.24 | A | S |
| ATOM | 3186 | C | CYS | A | 614 | 13.803 | −4.393 | 20.791 | 1.00 | 7.51 | A | C |
| ATOM | 3187 | O | CYS | A | 614 | 14.404 | −3.737 | 21.652 | 1.00 | 8.23 | A | O |
| ATOM | 3188 | N | MET | A | 615 | 13.602 | −5.700 | 20.939 | 1.00 | 6.99 | A | N |
| ATOM | 3190 | CA | MET | A | 615 | 14.136 | −6.415 | 22.104 | 1.00 | 8.46 | A | C |
| ATOM | 3192 | CB | MET | A | 615 | 13.727 | −7.893 | 22.087 | 1.00 | 8.91 | A | C |
| ATOM | 3195 | CG | MET | A | 615 | 12.273 | −8.132 | 22.309 | 1.00 | 11.03 | A | C |
| ATOM | 3198 | SD | MET | A | 615 | 12.003 | −9.900 | 22.802 | 1.00 | 14.28 | A | S |
| ATOM | 3199 | CE | MET | A | 615 | 12.231 | −10.684 | 21.273 | 1.00 | 13.21 | A | C |
| ATOM | 3203 | C | MET | A | 615 | 15.657 | −6.314 | 22.182 | 1.00 | 7.71 | A | C |
| ATOM | 3204 | O | MET | A | 615 | 16.223 | −6.111 | 23.249 | 1.00 | 8.82 | A | O |
| ATOM | 3205 | N | TRP | A | 616 | 16.324 | −6.420 | 21.042 | 1.00 | 7.61 | A | N |
| ATOM | 3207 | CA | TRP | A | 616 | 17.773 | −6.243 | 20.982 | 1.00 | 7.16 | A | C |
| ATOM | 3209 | CB | TRP | A | 616 | 18.277 | −6.483 | 19.555 | 1.00 | 7.70 | A | C |
| ATOM | 3212 | CG | TRP | A | 616 | 19.759 | −6.411 | 19.431 | 1.00 | 6.78 | A | C |
| ATOM | 3213 | CD1 | TRP | A | 616 | 20.623 | −7.465 | 19.493 | 1.00 | 7.50 | A | C |
| ATOM | 3215 | NE1 | TRP | A | 616 | 21.916 | −7.021 | 19.337 | 1.00 | 8.34 | A | N |
| ATOM | 3217 | CE2 | TRP | A | 616 | 21.913 | −5.665 | 19.176 | 1.00 | 8.17 | A | C |
| ATOM | 3218 | CD2 | TRP | A | 616 | 20.559 | −5.247 | 19.199 | 1.00 | 5.69 | A | C |
| ATOM | 3219 | CE3 | TRP | A | 616 | 20.285 | −3.876 | 19.074 | 1.00 | 8.29 | A | C |
| ATOM | 3221 | CZ3 | TRP | A | 616 | 21.327 | −3.003 | 18.884 | 1.00 | 8.52 | A | C |
| ATOM | 3223 | CH2 | TRP | A | 616 | 22.663 | −3.460 | 18.833 | 1.00 | 7.88 | A | C |
| ATOM | 3225 | CZ2 | TRP | A | 616 | 22.964 | −4.786 | 18.940 | 1.00 | 8.12 | A | C |
| ATOM | 3227 | C | TRP | A | 616 | 18.171 | −4.841 | 21.476 | 1.00 | 7.53 | A | C |
| ATOM | 3228 | O | TRP | A | 616 | 19.125 | −4.732 | 22.241 | 1.00 | 8.03 | A | O |
| ATOM | 3229 | N | GLU | A | 617 | 17.425 | −3.794 | 21.081 | 1.00 | 7.69 | A | N |
| ATOM | 3231 | CA | GLU | A | 617 | 17.671 | −2.434 | 21.555 | 1.00 | 8.18 | A | C |
| ATOM | 3233 | CB | GLU | A | 617 | 16.718 | −1.416 | 20.918 | 1.00 | 8.57 | A | C |
| ATOM | 3236 | CG | GLU | A | 617 | 16.871 | −1.110 | 19.441 | 1.00 | 10.70 | A | C |
| ATOM | 3239 | CD | GLU | A | 617 | 15.817 | −0.100 | 19.000 | 1.00 | 13.08 | A | C |
| ATOM | 3240 | OE1 | GLU | A | 617 | 16.176 | 0.998 | 18.510 | 1.00 | 13.59 | A | O |
| ATOM | 3241 | OE2 | GLU | A | 617 | 14.620 | −0.372 | 19.205 | 1.00 | 14.34 | A | O |
| ATOM | 3242 | C | GLU | A | 617 | 17.496 | −2.334 | 23.066 | 1.00 | 8.12 | A | C |
| ATOM | 3243 | O | GLU | A | 617 | 18.304 | −1.700 | 23.749 | 1.00 | 8.06 | A | O |
| ATOM | 3244 | N | ILE | A | 618 | 16.453 | −2.970 | 23.593 | 1.00 | 8.10 | A | N |
| ATOM | 3246 | CA | ILE | A | 618 | 16.172 | −2.899 | 25.027 | 1.00 | 8.31 | A | C |
| ATOM | 3248 | CB | ILE | A | 618 | 14.810 | −3.549 | 25.349 | 1.00 | 8.09 | A | C |
| ATOM | 3250 | CG1 | ILE | A | 618 | 13.676 | −2.696 | 24.783 | 1.00 | 7.50 | A | C |
| ATOM | 3253 | CD1 | ILE | A | 618 | 12.296 | −3.333 | 24.895 | 1.00 | 9.45 | A | C |
| ATOM | 3257 | CG2 | ILE | A | 618 | 14.641 | −3.746 | 26.867 | 1.00 | 9.02 | A | C |
| ATOM | 3261 | C | ILE | A | 618 | 17.314 | −3.569 | 25.805 | 1.00 | 8.52 | A | C |
| ATOM | 3262 | O | ILE | A | 618 | 17.884 | −2.983 | 26.731 | 1.00 | 9.07 | A | O |
| ATOM | 3263 | N | LEU | A | 619 | 17.674 | −4.780 | 25.406 | 1.00 | 9.16 | A | N |
| ATOM | 3265 | CA | LEU | A | 619 | 18.752 | −5.517 | 26.063 | 1.00 | 9.73 | A | C |
| ATOM | 3267 | CB | LEU | A | 619 | 18.742 | −6.989 | 25.626 | 1.00 | 10.15 | A | C |
| ATOM | 3270 | CG | LEU | A | 619 | 17.821 | −7.924 | 26.416 | 1.00 | 11.23 | A | C |
| ATOM | 3272 | CD1 | LEU | A | 619 | 18.274 | −8.131 | 27.852 | 1.00 | 12.73 | A | C |
| ATOM | 3276 | CD2 | LEU | A | 619 | 16.384 | −7.464 | 26.361 | 1.00 | 12.78 | A | C |
| ATOM | 3280 | C | LEU | A | 619 | 20.132 | −4.906 | 25.858 | 1.00 | 9.41 | A | C |
| ATOM | 3281 | O | LEU | A | 619 | 21.071 | −5.239 | 26.600 | 1.00 | 11.30 | A | O |
| ATOM | 3282 | N | SER | A | 620 | 20.249 | −3.996 | 24.890 | 1.00 | 9.34 | A | N |
| ATOM | 3284 | CA | SER | A | 620 | 21.466 | −3.226 | 24.656 | 1.00 | 9.57 | A | C |
| ATOM | 3286 | CB | SER | A | 620 | 21.748 | −3.159 | 23.147 | 1.00 | 9.27 | A | C |
| ATOM | 3289 | OG | SER | A | 620 | 21.720 | −4.438 | 22.539 | 1.00 | 9.62 | A | O |
| ATOM | 3291 | C | SER | A | 620 | 21.412 | −1.788 | 25.217 | 1.00 | 10.03 | A | C |
| ATOM | 3292 | O | SER | A | 620 | 22.253 | −0.958 | 24.870 | 1.00 | 10.13 | A | O |
| ATOM | 3293 | N | PHE | A | 621 | 20.438 | −1.495 | 26.072 | 1.00 | 10.21 | A | N |
| ATOM | 3295 | CA | PHE | A | 621 | 20.281 | −0.168 | 26.658 | 1.00 | 10.99 | A | C |
| ATOM | 3297 | CB | PHE | A | 621 | 21.380 | 0.092 | 27.686 | 1.00 | 11.57 | A | C |
| ATOM | 3300 | CG | PHE | A | 621 | 21.417 | −0.916 | 28.786 | 1.00 | 11.76 | A | C |
| ATOM | 3301 | CD1 | PHE | A | 621 | 20.585 | −0.777 | 29.903 | 1.00 | 14.73 | A | C |
| ATOM | 3303 | CE1 | PHE | A | 621 | 20.616 | −1.720 | 30.939 | 1.00 | 13.38 | A | C |
| ATOM | 3305 | CZ | PHE | A | 621 | 21.455 | −2.806 | 30.845 | 1.00 | 15.10 | A | C |
| ATOM | 3307 | CE2 | PHE | A | 621 | 22.282 | −2.966 | 29.728 | 1.00 | 15.45 | A | C |
| ATOM | 3309 | CD2 | PHE | A | 621 | 22.257 | −2.020 | 28.708 | 1.00 | 14.11 | A | C |
| ATOM | 3311 | C | PHE | A | 621 | 20.204 | 0.974 | 25.633 | 1.00 | 11.51 | A | C |
| ATOM | 3312 | O | PHE | A | 621 | 20.746 | 2.054 | 25.840 | 1.00 | 11.76 | A | O |
| ATOM | 3313 | N | GLY | A | 622 | 19.533 | 0.715 | 24.513 | 1.00 | 12.15 | A | N |
| ATOM | 3315 | CA | GLY | A | 622 | 19.195 | 1.755 | 23.561 | 1.00 | 12.52 | A | C |
| ATOM | 3318 | C | GLY | A | 622 | 20.125 | 1.958 | 22.389 | 1.00 | 13.31 | A | C |
| ATOM | 3319 | O | GLY | A | 622 | 19.957 | 2.916 | 21.631 | 1.00 | 13.27 | A | O |
| ATOM | 3320 | N | LYS | A | 623 | 21.117 | 1.086 | 22.224 | 1.00 | 13.94 | A | N |
| ATOM | 3322 | CA | LYS | A | 623 | 22.003 | 1.199 | 21.072 | 1.00 | 14.20 | A | C |
| ATOM | 3324 | CB | LYS | A | 623 | 23.120 | 0.167 | 21.156 | 1.00 | 15.30 | A | C |
| ATOM | 3327 | CG | LYS | A | 623 | 24.036 | 0.374 | 22.340 | 1.00 | 18.22 | A | C |
| ATOM | 3330 | CD | LYS | A | 623 | 25.359 | −0.349 | 22.137 | 1.00 | 23.13 | A | C |
| ATOM | 3333 | CE | LYS | A | 623 | 25.198 | −1.851 | 22.058 | 1.00 | 23.04 | A | C |
| ATOM | 3336 | NZ | LYS | A | 623 | 26.022 | −2.515 | 23.072 | 1.00 | 22.31 | A | N |
| ATOM | 3340 | C | LYS | A | 623 | 21.223 | 1.016 | 19.765 | 1.00 | 13.35 | A | C |
| ATOM | 3341 | O | LYS | A | 623 | 20.175 | 0.354 | 19.732 | 1.00 | 12.25 | A | O |
| ATOM | 3342 | N | GLN | A | 624 | 21.720 | 1.631 | 18.694 | 1.00 | 12.33 | A | N |
| ATOM | 3344 | CA | GLN | A | 624 | 21.074 | 1.532 | 17.390 | 1.00 | 11.79 | A | C |
| ATOM | 3346 | CB | GLN | A | 624 | 21.471 | 2.693 | 16.471 | 1.00 | 12.79 | A | C |
| ATOM | 3349 | CG | GLN | A | 624 | 20.803 | 2.653 | 15.092 | 1.00 | 16.84 | A | C |
| ATOM | 3352 | CD | GLN | A | 624 | 21.480 | 3.512 | 14.033 | 1.00 | 20.72 | A | C |
| ATOM | 3353 | OE1 | GLN | A | 624 | 22.510 | 3.139 | 13.484 | 1.00 | 22.00 | A | O |
| ATOM | 3354 | NE2 | GLN | A | 624 | 20.863 | 4.642 | 13.706 | 1.00 | 25.56 | A | N |
| ATOM | 3357 | C | GLN | A | 624 | 21.484 | 0.218 | 16.750 | 1.00 | 10.43 | A | C |
| ATOM | 3358 | O | GLN | A | 624 | 22.674 | −0.085 | 16.700 | 1.00 | 9.05 | A | O |
| ATOM | 3359 | N | PRO | A | 625 | 20.527 | −0.543 | 16.226 | 1.00 | 9.21 | A | N |
| ATOM | 3360 | CA | PRO | A | 625 | 20.857 | −1.773 | 15.510 | 1.00 | 8.85 | A | C |
| ATOM | 3362 | CB | PRO | A | 625 | 19.489 | −2.336 | 15.099 | 1.00 | 8.81 | A | C |
| ATOM | 3365 | CG | PRO | A | 625 | 18.589 | −1.182 | 15.135 | 1.00 | 8.12 | A | C |
| ATOM | 3368 | CD | PRO | A | 625 | 19.077 | −0.284 | 16.226 | 1.00 | 8.87 | A | C |
| ATOM | 3371 | C | PRO | A | 625 | 21.690 | −1.483 | 14.260 | 1.00 | 9.18 | A | C |
| ATOM | 3372 | O | PRO | A | 625 | 21.427 | −0.517 | 13.529 | 1.00 | 8.70 | A | O |
| ATOM | 3373 | N | PHE | A | 626 | 22.699 | −2.312 | 14.017 | 1.00 | 8.97 | A | N |
| ATOM | 3375 | CA | PHE | A | 626 | 23.530 | −2.167 | 12.825 | 1.00 | 8.80 | A | C |
| ATOM | 3377 | CB | PHE | A | 626 | 22.755 | −2.516 | 11.552 | 1.00 | 9.35 | A | C |
| ATOM | 3380 | CG | PHE | A | 626 | 22.317 | −3.959 | 11.476 | 1.00 | 7.57 | A | C |
| ATOM | 3381 | CD1 | PHE | A | 626 | 23.263 | −4.982 | 11.428 | 1.00 | 8.74 | A | C |
| ATOM | 3383 | CE1 | PHE | A | 626 | 22.880 | −6.293 | 11.333 | 1.00 | 7.90 | A | C |
| ATOM | 3385 | CZ | PHE | A | 626 | 21.544 | −6.621 | 11.266 | 1.00 | 9.02 | A | C |
| ATOM | 3387 | CE2 | PHE | A | 626 | 20.584 | −5.630 | 11.313 | 1.00 | 7.58 | A | C |
| ATOM | 3389 | CD2 | PHE | A | 626 | 20.967 | −4.303 | 11.402 | 1.00 | 7.59 | A | C |
| ATOM | 3391 | C | PHE | A | 626 | 24.153 | −0.762 | 12.737 | 1.00 | 9.81 | A | C |
| ATOM | 3392 | O | PHE | A | 626 | 24.281 | −0.193 | 11.669 | 1.00 | 9.56 | A | O |
| ATOM | 3393 | N | PHE | A | 627 | 24.554 | −0.231 | 13.879 | 1.00 | 9.82 | A | N |
| ATOM | 3395 | CA | PHE | A | 627 | 25.190 | 1.088 | 13.917 | 1.00 | 11.48 | A | C |
| ATOM | 3397 | CB | PHE | A | 627 | 25.448 | 1.543 | 15.365 | 1.00 | 11.15 | A | C |
| ATOM | 3400 | CG | PHE | A | 627 | 26.395 | 0.662 | 16.150 | 1.00 | 11.35 | A | C |
| ATOM | 3401 | CD1 | PHE | A | 627 | 27.774 | 0.810 | 16.042 | 1.00 | 9.33 | A | C |
| ATOM | 3403 | CE1 | PHE | A | 627 | 28.637 | 0.017 | 16.772 | 1.00 | 10.69 | A | C |
| ATOM | 3405 | CZ | PHE | A | 627 | 28.131 | −0.911 | 17.652 | 1.00 | 12.10 | A | C |
| ATOM | 3407 | CE2 | PHE | A | 627 | 26.746 | −1.042 | 17.787 | 1.00 | 10.84 | A | C |
| ATOM | 3409 | CD2 | PHE | A | 627 | 25.905 | −0.256 | 17.051 | 1.00 | 9.05 | A | C |
| ATOM | 3411 | C | PHE | A | 627 | 26.475 | 1.142 | 13.092 | 1.00 | 12.51 | A | C |
| ATOM | 3412 | O | PHE | A | 627 | 26.881 | 2.214 | 12.636 | 1.00 | 14.55 | A | O |
| ATOM | 3413 | N | TRP | A | 628 | 27.076 | −0.021 | 12.879 | 1.00 | 12.47 | A | N |
| ATOM | 3415 | CA | TRP | A | 628 | 28.355 | −0.158 | 12.189 | 1.00 | 13.92 | A | C |
| ATOM | 3417 | CB | TRP | A | 628 | 29.085 | −1.409 | 12.710 | 1.00 | 13.56 | A | C |
| ATOM | 3420 | CG | TRP | A | 628 | 28.298 | −2.730 | 12.615 | 1.00 | 13.24 | A | C |
| ATOM | 3421 | CD1 | TRP | A | 628 | 28.356 | −3.647 | 11.600 | 1.00 | 13.12 | A | C |
| ATOM | 3423 | NE1 | TRP | A | 628 | 27.528 | −4.710 | 11.871 | 1.00 | 14.92 | A | N |
| ATOM | 3425 | CE2 | TRP | A | 628 | 26.894 | −4.493 | 13.060 | 1.00 | 13.87 | A | C |
| ATOM | 3426 | CD2 | TRP | A | 628 | 27.366 | −3.256 | 13.566 | 1.00 | 12.08 | A | C |
| ATOM | 3427 | CE3 | TRP | A | 628 | 26.861 | −2.807 | 14.784 | 1.00 | 13.25 | A | C |
| ATOM | 3429 | CZ3 | TRP | A | 628 | 25.928 | −3.597 | 15.468 | 1.00 | 13.18 | A | C |
| ATOM | 3431 | CH2 | TRP | A | 628 | 25.490 | −4.812 | 14.935 | 1.00 | 12.66 | A | C |
| ATOM | 3433 | CZ2 | TRP | A | 628 | 25.966 | −5.276 | 13.739 | 1.00 | 12.17 | A | C |
| ATOM | 3435 | C | TRP | A | 628 | 28.204 | −0.203 | 10.658 | 1.00 | 14.82 | A | C |
| ATOM | 3436 | O | TRP | A | 628 | 29.199 | −0.297 | 9.938 | 1.00 | 16.19 | A | O |
| ATOM | 3437 | N | LEU | A | 629 | 26.963 | −0.177 | 10.171 | 1.00 | 14.95 | A | N |
| ATOM | 3439 | CA | LEU | A | 629 | 26.653 | −0.266 | 8.744 | 1.00 | 15.95 | A | C |
| ATOM | 3441 | CB | LEU | A | 629 | 25.711 | −1.447 | 8.475 | 1.00 | 15.63 | A | C |
| ATOM | 3444 | CG | LEU | A | 629 | 26.128 | −2.866 | 8.839 | 1.00 | 15.85 | A | C |
| ATOM | 3446 | CD1 | LEU | A | 629 | 25.034 | −3.791 | 8.377 | 1.00 | 17.66 | A | C |
| ATOM | 3450 | CD2 | LEU | A | 629 | 27.437 | −3.241 | 8.191 | 1.00 | 17.21 | A | C |
| ATOM | 3454 | C | LEU | A | 629 | 25.957 | 0.991 | 8.249 | 1.00 | 16.16 | A | C |
| ATOM | 3455 | O | LEU | A | 629 | 25.422 | 1.767 | 9.042 | 1.00 | 17.19 | A | O |
| ATOM | 3456 | N | GLU | A | 630 | 26.012 | 1.200 | 6.933 | 1.00 | 17.08 | A | N |
| ATOM | 3458 | CA | GLU | A | 630 | 25.159 | 2.176 | 6.244 | 1.00 | 17.66 | A | C |
| ATOM | 3460 | CB | GLU | A | 630 | 25.859 | 2.759 | 4.990 | 1.00 | 18.54 | A | C |
| ATOM | 3463 | CG | GLU | A | 630 | 27.107 | 3.592 | 5.285 | 1.00 | 22.16 | A | C |
| ATOM | 3466 | CD | GLU | A | 630 | 27.834 | 4.104 | 4.035 | 1.00 | 26.66 | A | C |
| ATOM | 3467 | OE1 | GLU | A | 630 | 28.025 | 3.347 | 3.054 | 1.00 | 30.10 | A | O |
| ATOM | 3468 | OE2 | GLU | A | 630 | 28.254 | 5.276 | 4.041 | 1.00 | 31.44 | A | O |
| ATOM | 3469 | C | GLU | A | 630 | 23.877 | 1.444 | 5.840 | 1.00 | 16.80 | A | C |
| ATOM | 3470 | O | GLU | A | 630 | 23.899 | 0.232 | 5.644 | 1.00 | 16.52 | A | O |
| ATOM | 3471 | N | ASN | A | 631 | 22.777 | 2.177 | 5.693 | 1.00 | 16.64 | A | N |
| ATOM | 3473 | CA | ASN | A | 631 | 21.494 | 1.588 | 5.288 | 1.00 | 16.86 | A | C |
| ATOM | 3475 | CB | ASN | A | 631 | 20.444 | 2.692 | 5.027 | 1.00 | 16.92 | A | C |
| ATOM | 3478 | CG | ASN | A | 631 | 19.758 | 3.184 | 6.315 | 1.00 | 18.23 | A | C |
| ATOM | 3479 | OD1 | ASN | A | 631 | 19.981 | 2.645 | 7.395 | 1.00 | 19.71 | A | O |
| ATOM | 3480 | ND2 | ASN | A | 631 | 18.935 | 4.230 | 6.196 | 1.00 | 18.99 | A | N |
| ATOM | 3483 | C | ASN | A | 631 | 21.626 | 0.674 | 4.067 | 1.00 | 16.54 | A | C |
| ATOM | 3484 | O | ASN | A | 631 | 21.011 | −0.395 | 4.008 | 1.00 | 16.91 | A | O |
| ATOM | 3485 | N | LYS | A | 632 | 22.435 | 1.094 | 3.097 | 1.00 | 17.10 | A | N |
| ATOM | 3487 | CA | LYS | A | 632 | 22.531 | 0.396 | 1.817 | 1.00 | 17.56 | A | C |
| ATOM | 3489 | CB | LYS | A | 632 | 23.263 | 1.260 | 0.770 | 1.00 | 17.78 | A | C |
| ATOM | 3492 | CG | LYS | A | 632 | 24.756 | 1.461 | 0.990 | 1.00 | 19.40 | A | C |
| ATOM | 3495 | CD | LYS | A | 632 | 25.297 | 2.438 | −0.081 | 1.00 | 23.92 | A | C |
| ATOM | 3498 | CE | LYS | A | 632 | 26.787 | 2.688 | 0.044 | 1.00 | 24.81 | A | C |
| ATOM | 3501 | NZ | LYS | A | 632 | 27.592 | 1.430 | 0.014 | 1.00 | 27.18 | A | N |
| ATOM | 3505 | C | LYS | A | 632 | 23.194 | −0.965 | 1.940 | 1.00 | 17.38 | A | C |
| ATOM | 3506 | O | LYS | A | 632 | 23.086 | −1.798 | 1.040 | 1.00 | 17.61 | A | O |
| ATOM | 3507 | N | ASP | A | 633 | 23.863 | −1.195 | 3.064 | 1.00 | 17.62 | A | N |
| ATOM | 3509 | CA | ASP | A | 633 | 24.618 | −2.423 | 3.287 | 1.00 | 17.79 | A | C |
| ATOM | 3511 | CB | ASP | A | 633 | 25.892 | −2.111 | 4.070 | 1.00 | 18.50 | A | C |
| ATOM | 3514 | CG | ASP | A | 633 | 26.869 | −1.242 | 3.289 | 1.00 | 22.13 | A | C |
| ATOM | 3515 | OD1 | ASP | A | 633 | 27.019 | −1.425 | 2.062 | 1.00 | 25.82 | A | O |
| ATOM | 3516 | OD2 | ASP | A | 633 | 27.534 | −0.344 | 3.843 | 1.00 | 29.08 | A | O |
| ATOM | 3517 | C | ASP | A | 633 | 23.830 | −3.477 | 4.054 | 1.00 | 16.35 | A | C |
| ATOM | 3518 | O | ASP | A | 633 | 24.244 | −4.631 | 4.106 | 1.00 | 16.00 | A | O |
| ATOM | 3519 | N | VAL | A | 634 | 22.698 | −3.086 | 4.639 | 1.00 | 14.77 | A | N |
| ATOM | 3521 | CA | VAL | A | 634 | 21.941 | −3.962 | 5.535 | 1.00 | 14.11 | A | C |
| ATOM | 3523 | CB | VAL | A | 634 | 20.767 | −3.188 | 6.199 | 1.00 | 14.20 | A | C |
| ATOM | 3525 | CG1 | VAL | A | 634 | 19.834 | −4.110 | 6.957 | 1.00 | 14.96 | A | C |
| ATOM | 3529 | CG2 | VAL | A | 634 | 21.298 | −2.120 | 7.131 | 1.00 | 15.06 | A | C |
| ATOM | 3533 | C | VAL | A | 634 | 21.448 | −5.224 | 4.823 | 1.00 | 13.43 | A | C |
| ATOM | 3534 | O | VAL | A | 634 | 21.685 | −6.339 | 5.289 | 1.00 | 12.23 | A | O |
| ATOM | 3535 | N | ILE | A | 635 | 20.792 | −5.063 | 3.676 | 1.00 | 13.66 | A | N |
| ATOM | 3537 | CA | ILE | A | 635 | 20.175 | −6.214 | 3.010 | 1.00 | 13.87 | A | C |
| ATOM | 3539 | CB | ILE | A | 635 | 19.308 | −5.792 | 1.786 | 1.00 | 14.01 | A | C |
| ATOM | 3541 | CG1 | ILE | A | 635 | 18.407 | −6.942 | 1.344 | 1.00 | 13.90 | A | C |
| ATOM | 3544 | CD1 | ILE | A | 635 | 17.406 | −7.408 | 2.394 | 1.00 | 14.48 | A | C |
| ATOM | 3548 | CG2 | ILE | A | 635 | 20.169 | −5.256 | 0.628 | 1.00 | 14.55 | A | C |
| ATOM | 3552 | C | ILE | A | 635 | 21.205 | −7.287 | 2.666 | 1.00 | 14.30 | A | C |
| ATOM | 3553 | O | ILE | A | 635 | 20.961 | −8.467 | 2.912 | 1.00 | 13.32 | A | O |
| ATOM | 3554 | N | GLY | A | 636 | 22.369 | −6.865 | 2.167 | 1.00 | 14.75 | A | N |
| ATOM | 3556 | CA | GLY | A | 636 | 23.483 | −7.771 | 1.868 | 1.00 | 14.87 | A | C |
| ATOM | 3559 | C | GLY | A | 636 | 23.898 | −8.630 | 3.048 | 1.00 | 14.75 | A | C |
| ATOM | 3560 | O | GLY | A | 636 | 24.068 | −9.846 | 2.937 | 1.00 | 14.77 | A | O |
| ATOM | 3561 | N | VAL | A | 637 | 24.046 | −7.989 | 4.195 | 1.00 | 14.62 | A | N |
| ATOM | 3563 | CA | VAL | A | 637 | 24.396 | −8.669 | 5.431 | 1.00 | 14.71 | A | C |
| ATOM | 3565 | CB | VAL | A | 637 | 24.620 | −7.610 | 6.548 | 1.00 | 15.24 | A | C |
| ATOM | 3567 | CG1 | VAL | A | 637 | 24.587 | −8.215 | 7.928 | 1.00 | 15.93 | A | C |
| ATOM | 3571 | CG2 | VAL | A | 637 | 25.932 | −6.869 | 6.295 | 1.00 | 16.95 | A | C |
| ATOM | 3575 | C | VAL | A | 637 | 23.336 | −9.700 | 5.841 | 1.00 | 14.05 | A | C |
| ATOM | 3576 | O | VAL | A | 637 | 23.644 | −10.829 | 6.188 | 1.00 | 13.96 | A | O |
| ATOM | 3577 | N | LEU | A | 638 | 22.074 | −9.322 | 5.779 | 1.00 | 13.42 | A | N |
| ATOM | 3579 | CA | LEU | A | 638 | 20.995 | −10.231 | 6.146 | 1.00 | 13.52 | A | C |
| ATOM | 3581 | CB | LEU | A | 638 | 19.669 | −9.472 | 6.179 | 1.00 | 13.37 | A | C |
| ATOM | 3584 | CG | LEU | A | 638 | 19.587 | −8.329 | 7.203 | 1.00 | 12.38 | A | C |
| ATOM | 3586 | CD1 | LEU | A | 638 | 18.240 | −7.644 | 7.078 | 1.00 | 12.99 | A | C |
| ATOM | 3590 | CD2 | LEU | A | 638 | 19.783 | −8.855 | 8.587 | 1.00 | 13.05 | A | C |
| ATOM | 3594 | C | LEU | A | 638 | 20.902 | −11.427 | 5.188 | 1.00 | 14.41 | A | C |
| ATOM | 3595 | O | LEU | A | 638 | 20.640 | −12.547 | 5.617 | 1.00 | 13.64 | A | O |
| ATOM | 3596 | N | GLU | A | 639 | 21.119 | −11.170 | 3.897 | 1.00 | 15.99 | A | N |
| ATOM | 3598 | CA | GLU | A | 639 | 21.032 | −12.204 | 2.860 | 1.00 | 17.38 | A | C |
| ATOM | 3600 | CB | GLU | A | 639 | 20.974 | −11.585 | 1.446 | 1.00 | 17.84 | A | C |
| ATOM | 3603 | CG | GLU | A | 639 | 19.567 | −11.053 | 1.155 | 1.00 | 19.95 | A | C |
| ATOM | 3606 | CD | GLU | A | 639 | 19.349 | −10.385 | −0.196 | 1.00 | 24.27 | A | C |
| ATOM | 3607 | OE1 | GLU | A | 639 | 20.314 | −9.998 | −0.890 | 1.00 | 26.43 | A | O |
| ATOM | 3608 | OE2 | GLU | A | 639 | 18.156 | −10.234 | −0.555 | 1.00 | 28.41 | A | O |
| ATOM | 3609 | C | GLU | A | 639 | 22.171 | −13.190 | 3.015 | 1.00 | 18.02 | A | C |
| ATOM | 3610 | O | GLU | A | 639 | 21.998 | −14.362 | 2.755 | 1.00 | 18.32 | A | O |
| ATOM | 3611 | N | LYS | A | 640 | 23.319 | −12.733 | 3.503 | 1.00 | 18.92 | A | N |
| ATOM | 3613 | CA | LYS | A | 640 | 24.431 | −13.645 | 3.751 | 1.00 | 19.98 | A | C |
| ATOM | 3615 | CB | LYS | A | 640 | 25.777 | −12.903 | 3.693 | 1.00 | 20.94 | A | C |
| ATOM | 3618 | CG | LYS | A | 640 | 26.233 | −12.229 | 4.965 | 1.00 | 24.88 | A | C |
| ATOM | 3621 | CD | LYS | A | 640 | 27.432 | −11.309 | 4.704 | 1.00 | 28.51 | A | C |
| ATOM | 3624 | CE | LYS | A | 640 | 28.699 | −12.102 | 4.445 | 1.00 | 30.97 | A | C |
| ATOM | 3627 | NZ | LYS | A | 640 | 29.922 | −11.339 | 4.842 | 1.00 | 32.88 | A | N |
| ATOM | 3631 | C | LYS | A | 640 | 24.244 | −14.456 | 5.038 | 1.00 | 19.37 | A | C |
| ATOM | 3632 | O | LYS | A | 640 | 25.047 | −15.343 | 5.324 | 1.00 | 20.32 | A | O |
| ATOM | 3633 | N | GLY | A | 641 | 23.172 | −14.188 | 5.795 | 1.00 | 18.02 | A | N |
| ATOM | 3635 | CA | GLY | A | 641 | 22.864 | −14.947 | 7.006 | 1.00 | 17.51 | A | C |
| ATOM | 3638 | C | GLY | A | 641 | 23.309 | −14.328 | 8.324 | 1.00 | 16.94 | A | C |
| ATOM | 3639 | O | GLY | A | 641 | 23.063 | −14.876 | 9.398 | 1.00 | 17.26 | A | O |
| ATOM | 3640 | N | ASP | A | 642 | 23.978 | −13.186 | 8.260 | 1.00 | 15.83 | A | N |
| ATOM | 3642 | CA | ASP | A | 642 | 24.426 | −12.516 | 9.476 | 1.00 | 15.01 | A | C |
| ATOM | 3644 | CB | ASP | A | 642 | 25.482 | −11.476 | 9.155 | 1.00 | 15.19 | A | C |
| ATOM | 3647 | CG | ASP | A | 642 | 26.786 | −12.079 | 8.651 | 1.00 | 18.80 | A | C |
| ATOM | 3648 | OD1 | ASP | A | 642 | 27.065 | −13.269 | 8.921 | 1.00 | 19.61 | A | O |
| ATOM | 3649 | OD2 | ASP | A | 642 | 27.586 | −11.400 | 7.975 | 1.00 | 23.88 | A | O |
| ATOM | 3650 | C | ASP | A | 642 | 23.249 | −11.815 | 10.152 | 1.00 | 13.57 | A | C |
| ATOM | 3651 | O | ASP | A | 642 | 22.307 | −11.354 | 9.490 | 1.00 | 12.33 | A | O |
| ATOM | 3652 | N | ARG | A | 643 | 23.362 | −11.697 | 11.464 | 1.00 | 12.50 | A | N |
| ATOM | 3654 | CA | ARG | A | 643 | 22.331 | −11.111 | 12.317 | 1.00 | 12.39 | A | C |
| ATOM | 3656 | CB | ARG | A | 643 | 21.489 | −12.225 | 12.953 | 1.00 | 12.39 | A | C |
| ATOM | 3659 | CG | ARG | A | 643 | 20.694 | −13.090 | 11.983 | 1.00 | 12.03 | A | C |
| ATOM | 3662 | CD | ARG | A | 643 | 19.623 | −12.348 | 11.205 | 1.00 | 11.99 | A | C |
| ATOM | 3665 | NE | ARG | A | 643 | 18.815 | −13.225 | 10.362 | 1.00 | 13.20 | A | N |
| ATOM | 3667 | CZ | ARG | A | 643 | 19.009 | −13.447 | 9.066 | 1.00 | 12.12 | A | C |
| ATOM | 3668 | NH1 | ARG | A | 643 | 20.017 | −12.891 | 8.422 | 1.00 | 12.02 | A | N |
| ATOM | 3671 | NH2 | ARG | A | 643 | 18.192 | −14.246 | 8.407 | 1.00 | 13.42 | A | N |
| ATOM | 3674 | C | ARG | A | 643 | 22.971 | −10.248 | 13.403 | 1.00 | 11.47 | A | C |
| ATOM | 3675 | O | ARG | A | 643 | 24.193 | −10.287 | 13.620 | 1.00 | 12.13 | A | O |
| ATOM | 3676 | N | LEU | A | 644 | 22.153 | −9.467 | 14.099 | 1.00 | 10.94 | A | N |
| ATOM | 3678 | CA | LEU | A | 644 | 22.594 | −8.737 | 15.290 | 1.00 | 10.40 | A | C |
| ATOM | 3680 | CB | LEU | A | 644 | 21.419 | −7.958 | 15.904 | 1.00 | 9.69 | A | C |
| ATOM | 3683 | CG | LEU | A | 644 | 20.852 | −6.832 | 15.018 | 1.00 | 11.08 | A | C |
| ATOM | 3685 | CD1 | LEU | A | 644 | 19.472 | −6.365 | 15.508 | 1.00 | 9.53 | A | C |
| ATOM | 3689 | CD2 | LEU | A | 644 | 21.813 | −5.666 | 14.973 | 1.00 | 10.40 | A | C |
| ATOM | 3693 | C | LEU | A | 644 | 23.159 | −9.720 | 16.313 | 1.00 | 10.18 | A | C |
| ATOM | 3694 | O | LEU | A | 644 | 22.586 | −10.783 | 16.529 | 1.00 | 9.49 | A | O |
| ATOM | 3695 | N | PRO | A | 645 | 24.294 | −9.404 | 16.929 | 1.00 | 11.40 | A | N |
| ATOM | 3696 | CA | PRO | A | 645 | 24.890 | −10.317 | 17.908 | 1.00 | 11.04 | A | C |
| ATOM | 3698 | CB | PRO | A | 645 | 26.306 | −9.773 | 18.072 | 1.00 | 11.53 | A | C |
| ATOM | 3701 | CG | PRO | A | 645 | 26.133 | −8.323 | 17.893 | 1.00 | 12.78 | A | C |
| ATOM | 3704 | CD | PRO | A | 645 | 25.098 | −8.180 | 16.770 | 1.00 | 11.64 | A | C |
| ATOM | 3707 | C | PRO | A | 645 | 24.153 | −10.280 | 19.237 | 1.00 | 10.76 | A | C |
| ATOM | 3708 | O | PRO | A | 645 | 23.433 | −9.317 | 19.545 | 1.00 | 10.19 | A | O |
| ATOM | 3709 | N | LYS | A | 646 | 24.334 | −11.309 | 20.050 | 1.00 | 10.57 | A | N |
| ATOM | 3711 | CA | LYS | A | 646 | 23.713 | −11.298 | 21.372 | 1.00 | 11.04 | A | C |
| ATOM | 3713 | CB | LYS | A | 646 | 23.920 | −12.626 | 22.090 | 1.00 | 11.87 | A | C |
| ATOM | 3716 | CG | LYS | A | 646 | 23.106 | −12.693 | 23.388 | 1.00 | 12.94 | A | C |
| ATOM | 3719 | CD | LYS | A | 646 | 23.208 | −14.023 | 24.094 | 1.00 | 15.55 | A | C |
| ATOM | 3722 | CE | LYS | A | 646 | 24.549 | −14.181 | 24.784 | 1.00 | 17.68 | A | C |
| ATOM | 3725 | NZ | LYS | A | 646 | 24.744 | −13.211 | 25.909 | 1.00 | 17.61 | A | N |
| ATOM | 3729 | C | LYS | A | 646 | 24.245 | −10.171 | 22.262 | 1.00 | 11.04 | A | C |
| ATOM | 3730 | O | LYS | A | 646 | 25.455 | −10.098 | 22.512 | 1.00 | 10.00 | A | O |
| ATOM | 3731 | N | PRO | A | 647 | 23.367 | −9.298 | 22.764 | 1.00 | 11.04 | A | N |
| ATOM | 3732 | CA | PRO | A | 647 | 23.795 | −8.304 | 23.753 | 1.00 | 11.70 | A | C |
| ATOM | 3734 | CB | PRO | A | 647 | 22.493 | −7.562 | 24.101 | 1.00 | 11.68 | A | C |
| ATOM | 3737 | CG | PRO | A | 647 | 21.613 | −7.755 | 22.933 | 1.00 | 10.24 | A | C |
| ATOM | 3740 | CD | PRO | A | 647 | 21.939 | −9.134 | 22.407 | 1.00 | 10.94 | A | C |
| ATOM | 3743 | C | PRO | A | 647 | 24.385 | −9.009 | 24.989 | 1.00 | 12.95 | A | C |
| ATOM | 3744 | O | PRO | A | 647 | 23.903 | −10.073 | 25.362 | 1.00 | 13.55 | A | O |
| ATOM | 3745 | N | ASP | A | 648 | 25.404 | −8.419 | 25.596 | 1.00 | 14.13 | A | N |
| ATOM | 3747 | CA | ASP | A | 648 | 26.079 | −9.025 | 26.746 | 1.00 | 15.83 | A | C |
| ATOM | 3749 | CB | ASP | A | 648 | 27.078 | −8.037 | 27.343 | 1.00 | 16.30 | A | C |
| ATOM | 3752 | CG | ASP | A | 648 | 27.957 | −8.685 | 28.387 | 1.00 | 19.77 | A | C |
| ATOM | 3753 | OD1 | ASP | A | 648 | 28.581 | −9.737 | 28.087 | 1.00 | 23.36 | A | O |
| ATOM | 3754 | OD2 | ASP | A | 648 | 28.045 | −8.239 | 29.538 | 1.00 | 22.83 | A | O |
| ATOM | 3755 | C | ASP | A | 648 | 25.132 | −9.530 | 27.867 | 1.00 | 15.47 | A | C |
| ATOM | 3756 | O | ASP | A | 648 | 25.328 | −10.620 | 28.405 | 1.00 | 15.60 | A | O |
| ATOM | 3757 | N | LEU | A | 649 | 24.107 | −8.758 | 28.208 | 1.00 | 15.04 | A | N |
| ATOM | 3759 | CA | LEU | A | 649 | 23.198 | −9.145 | 29.293 | 1.00 | 15.54 | A | C |
| ATOM | 3761 | CB | LEU | A | 649 | 22.786 | −7.917 | 30.102 | 1.00 | 16.16 | A | C |
| ATOM | 3764 | CG | LEU | A | 649 | 23.972 | −7.268 | 30.829 | 1.00 | 18.63 | A | C |
| ATOM | 3766 | CD1 | LEU | A | 649 | 23.508 | −6.172 | 31.752 | 1.00 | 21.84 | A | C |
| ATOM | 3770 | CD2 | LEU | A | 649 | 24.819 | −8.285 | 31.590 | 1.00 | 20.44 | A | C |
| ATOM | 3774 | C | LEU | A | 649 | 21.958 | −9.928 | 28.864 | 1.00 | 15.26 | A | C |
| ATOM | 3775 | O | LEU | A | 649 | 21.143 | −10.329 | 29.702 | 1.00 | 15.43 | A | O |
| ATOM | 3776 | N | CYS | A | 650 | 21.816 | −10.172 | 27.573 | 1.00 | 14.41 | A | N |
| ATOM | 3778 | CA | CYS | A | 650 | 20.722 | −10.976 | 27.078 | 1.00 | 14.24 | A | C |
| ATOM | 3780 | CB | CYS | A | 650 | 20.630 | −10.821 | 25.562 | 1.00 | 14.58 | A | C |
| ATOM | 3783 | SG | CYS | A | 650 | 19.263 | −11.720 | 24.860 | 1.00 | 14.34 | A | S |
| ATOM | 3784 | C | CYS | A | 650 | 20.886 | −12.462 | 27.463 | 1.00 | 14.97 | A | C |
| ATOM | 3785 | O | CYS | A | 650 | 21.918 | −13.062 | 27.147 | 1.00 | 14.59 | A | O |
| ATOM | 3786 | N | PRO | A | 651 | 19.882 | −13.046 | 28.131 | 1.00 | 15.29 | A | N |
| ATOM | 3787 | CA | PRO | A | 651 | 19.881 | −14.480 | 28.453 | 1.00 | 15.51 | A | C |
| ATOM | 3789 | CB | PRO | A | 651 | 18.505 | −14.690 | 29.096 | 1.00 | 15.33 | A | C |
| ATOM | 3792 | CG | PRO | A | 651 | 18.118 | −13.377 | 29.606 | 1.00 | 16.20 | A | C |
| ATOM | 3795 | CD | PRO | A | 651 | 18.647 | −12.395 | 28.606 | 1.00 | 15.45 | A | C |
| ATOM | 3798 | C | PRO | A | 651 | 19.950 | −15.280 | 27.156 | 1.00 | 15.61 | A | C |
| ATOM | 3799 | O | PRO | A | 651 | 19.232 | −14.936 | 26.226 | 1.00 | 13.93 | A | O |
| ATOM | 3800 | N | PRO | A | 652 | 20.796 | −16.304 | 27.069 | 1.00 | 15.46 | A | N |
| ATOM | 3801 | CA | PRO | A | 652 | 20.857 | −17.144 | 25.865 | 1.00 | 15.22 | A | C |
| ATOM | 3803 | CB | PRO | A | 652 | 21.714 | −18.328 | 26.322 | 1.00 | 16.43 | A | C |
| ATOM | 3806 | CG | PRO | A | 652 | 22.606 | −17.704 | 27.397 | 1.00 | 15.77 | A | C |
| ATOM | 3809 | CD | PRO | A | 652 | 21.777 | −16.707 | 28.093 | 1.00 | 16.56 | A | C |
| ATOM | 3812 | C | PRO | A | 652 | 19.492 | −17.602 | 25.303 | 1.00 | 14.66 | A | C |
| ATOM | 3813 | O | PRO | A | 652 | 19.290 | −17.564 | 24.078 | 1.00 | 13.44 | A | O |
| ATOM | 3814 | N | VAL | A | 653 | 18.583 | −18.024 | 26.175 | 1.00 | 14.07 | A | N |
| ATOM | 3816 | CA | VAL | A | 653 | 17.232 | −18.429 | 25.766 | 1.00 | 14.45 | A | C |
| ATOM | 3818 | CB | VAL | A | 653 | 16.422 | −18.968 | 26.990 | 1.00 | 15.04 | A | C |
| ATOM | 3820 | CG1 | VAL | A | 653 | 16.178 | −17.897 | 28.039 | 1.00 | 16.41 | A | C |
| ATOM | 3824 | CG2 | VAL | A | 653 | 15.103 | −19.588 | 26.565 | 1.00 | 16.73 | A | C |
| ATOM | 3828 | C | VAL | A | 653 | 16.476 | −17.297 | 25.056 | 1.00 | 13.12 | A | C |
| ATOM | 3829 | O | VAL | A | 653 | 15.724 | −17.519 | 24.101 | 1.00 | 13.27 | A | O |
| ATOM | 3830 | N | LEU | A | 654 | 16.671 | −16.075 | 25.525 | 1.00 | 12.25 | A | N |
| ATOM | 3832 | CA | LEU | A | 654 | 16.061 | −14.930 | 24.859 | 1.00 | 11.55 | A | C |
| ATOM | 3834 | CB | LEU | A | 654 | 16.133 | −13.699 | 25.762 | 1.00 | 12.25 | A | C |
| ATOM | 3837 | CG | LEU | A | 654 | 15.424 | −12.465 | 25.211 | 1.00 | 11.73 | A | C |
| ATOM | 3839 | CD1 | LEU | A | 654 | 13.992 | −12.763 | 24.836 | 1.00 | 12.71 | A | C |
| ATOM | 3843 | CD2 | LEU | A | 654 | 15.497 | −11.336 | 26.213 | 1.00 | 15.67 | A | C |
| ATOM | 3847 | C | LEU | A | 654 | 16.705 | −14.659 | 23.499 | 1.00 | 11.43 | A | C |
| ATOM | 3848 | O | LEU | A | 654 | 16.017 | −14.299 | 22.532 | 1.00 | 9.70 | A | O |
| ATOM | 3849 | N | TYR | A | 655 | 18.023 | −14.819 | 23.391 | 1.00 | 10.65 | A | N |
| ATOM | 3851 | CA | TYR | A | 655 | 18.650 | −14.626 | 22.089 | 1.00 | 10.82 | A | C |
| ATOM | 3853 | CB | TYR | A | 655 | 20.170 | −14.605 | 22.197 | 1.00 | 10.18 | A | C |
| ATOM | 3856 | CG | TYR | A | 655 | 20.847 | −14.244 | 20.909 | 1.00 | 10.71 | A | C |
| ATOM | 3857 | CD1 | TYR | A | 655 | 20.691 | −12.978 | 20.344 | 1.00 | 9.23 | A | C |
| ATOM | 3859 | CE1 | TYR | A | 655 | 21.328 | −12.648 | 19.126 | 1.00 | 10.02 | A | C |
| ATOM | 3861 | CZ | TYR | A | 655 | 22.107 | −13.582 | 18.493 | 1.00 | 10.45 | A | C |
| ATOM | 3862 | OH | TYR | A | 655 | 22.732 | −13.275 | 17.313 | 1.00 | 11.48 | A | O |
| ATOM | 3864 | CE2 | TYR | A | 655 | 22.257 | −14.848 | 19.030 | 1.00 | 11.87 | A | C |
| ATOM | 3866 | CD2 | TYR | A | 655 | 21.607 | −15.175 | 20.222 | 1.00 | 11.48 | A | C |
| ATOM | 3868 | C | TYR | A | 655 | 18.159 | −15.669 | 21.083 | 1.00 | 11.69 | A | C |
| ATOM | 3869 | O | TYR | A | 655 | 17.945 | −15.356 | 19.924 | 1.00 | 13.15 | A | O |
| ATOM | 3870 | N | THR | A | 656 | 17.913 | −16.896 | 21.538 | 1.00 | 12.50 | A | N |
| ATOM | 3872 | CA | THR | A | 656 | 17.343 | −17.927 | 20.681 | 1.00 | 12.57 | A | C |
| ATOM | 3874 | CB | THR | A | 656 | 17.203 | −19.233 | 21.454 | 1.00 | 12.96 | A | C |
| ATOM | 3876 | OG1 | THR | A | 656 | 18.524 | −19.752 | 21.709 | 1.00 | 13.90 | A | O |
| ATOM | 3878 | CG2 | THR | A | 656 | 16.497 | −20.292 | 20.634 | 1.00 | 13.68 | A | C |
| ATOM | 3882 | C | THR | A | 656 | 16.002 | −17.478 | 20.123 | 1.00 | 12.60 | A | C |
| ATOM | 3883 | O | THR | A | 656 | 15.725 | −17.642 | 18.942 | 1.00 | 12.69 | A | O |
| ATOM | 3884 | N | LEU | A | 657 | 15.196 | −16.876 | 20.968 | 1.00 | 12.41 | A | N |
| ATOM | 3886 | CA | LEU | A | 657 | 13.896 | −16.387 | 20.550 | 1.00 | 13.31 | A | C |
| ATOM | 3888 | CB | LEU | A | 657 | 13.159 | −15.855 | 21.767 | 1.00 | 13.71 | A | C |
| ATOM | 3891 | CG | LEU | A | 657 | 11.651 | −15.834 | 21.701 | 1.00 | 17.10 | A | C |
| ATOM | 3893 | CD1 | LEU | A | 657 | 11.129 | −17.245 | 21.475 | 1.00 | 17.52 | A | C |
| ATOM | 3897 | CD2 | LEU | A | 657 | 11.093 | −15.233 | 22.999 | 1.00 | 17.71 | A | C |
| ATOM | 3901 | C | LEU | A | 657 | 14.035 | −15.306 | 19.478 | 1.00 | 12.69 | A | C |
| ATOM | 3902 | O | LEU | A | 657 | 13.318 | −15.328 | 18.471 | 1.00 | 11.85 | A | O |
| ATOM | 3903 | N | MET | A | 658 | 14.973 | −14.381 | 19.681 | 1.00 | 12.08 | A | N |
| ATOM | 3905 | CA | MET | A | 658 | 15.256 | −13.329 | 18.704 | 1.00 | 12.19 | A | C |
| ATOM | 3907 | CB | MET | A | 658 | 16.394 | −12.406 | 19.158 | 1.00 | 12.40 | A | C |
| ATOM | 3910 | CG | MET | A | 658 | 16.108 | −11.534 | 20.375 | 1.00 | 14.24 | A | C |
| ATOM | 3913 | SD | MET | A | 658 | 17.644 | −10.690 | 20.859 | 1.00 | 17.59 | A | S |
| ATOM | 3914 | CE | MET | A | 658 | 17.109 | −9.824 | 22.311 | 1.00 | 17.80 | A | C |
| ATOM | 3918 | C | MET | A | 658 | 15.622 | −13.928 | 17.361 | 1.00 | 11.84 | A | C |
| ATOM | 3919 | O | MET | A | 658 | 15.138 | −13.476 | 16.317 | 1.00 | 11.75 | A | O |
| ATOM | 3920 | N | THR | A | 659 | 16.467 | −14.964 | 17.370 | 1.00 | 11.94 | A | N |
| ATOM | 3922 | CA | THR | A | 659 | 16.955 | −15.520 | 16.113 | 1.00 | 12.12 | A | C |
| ATOM | 3924 | CB | THR | A | 659 | 18.149 | −16.515 | 16.292 | 1.00 | 12.54 | A | C |
| ATOM | 3926 | OG1 | THR | A | 659 | 17.762 | −17.627 | 17.096 | 1.00 | 16.72 | A | O |
| ATOM | 3928 | CG2 | THR | A | 659 | 19.287 | −15.907 | 17.045 | 1.00 | 12.86 | A | C |
| ATOM | 3932 | C | THR | A | 659 | 15.821 | −16.169 | 15.321 | 1.00 | 11.44 | A | C |
| ATOM | 3933 | O | THR | A | 659 | 15.857 | −16.155 | 14.085 | 1.00 | 11.07 | A | O |
| ATOM | 3934 | N | ARG | A | 660 | 14.828 | −16.730 | 16.026 | 1.00 | 11.23 | A | N |
| ATOM | 3936 | CA | ARG | A | 660 | 13.648 | −17.313 | 15.390 | 1.00 | 11.85 | A | C |
| ATOM | 3938 | CB | ARG | A | 660 | 12.801 | −18.090 | 16.395 | 1.00 | 12.33 | A | C |
| ATOM | 3941 | CG | ARG | A | 660 | 13.442 | −19.396 | 16.829 | 1.00 | 15.41 | A | C |
| ATOM | 3944 | CD | ARG | A | 660 | 12.668 | −20.082 | 17.920 | 1.00 | 18.66 | A | C |
| ATOM | 3947 | NE | ARG | A | 660 | 11.342 | −20.465 | 17.435 | 1.00 | 21.96 | A | N |
| ATOM | 3949 | CZ | ARG | A | 660 | 10.238 | −20.507 | 18.175 | 1.00 | 24.62 | A | C |
| ATOM | 3950 | NH1 | ARG | A | 660 | 10.262 | −20.224 | 19.484 | 1.00 | 25.28 | A | N |
| ATOM | 3953 | NH2 | ARG | A | 660 | 9.104 | −20.872 | 17.604 | 1.00 | 25.04 | A | N |
| ATOM | 3956 | C | ARG | A | 660 | 12.790 | −16.249 | 14.711 | 1.00 | 11.14 | A | C |
| ATOM | 3957 | O | ARG | A | 660 | 12.256 | −16.473 | 13.614 | 1.00 | 11.17 | A | O |
| ATOM | 3958 | N | CYS | A | 661 | 12.695 | −15.082 | 15.345 | 1.00 | 10.45 | A | N |
| ATOM | 3960 | CA | CYS | A | 661 | 12.009 | −13.926 | 14.756 | 1.00 | 10.32 | A | C |
| ATOM | 3962 | CB | CYS | A | 661 | 11.839 | −12.812 | 15.791 | 1.00 | 10.37 | A | C |
| ATOM | 3965 | SG | CYS | A | 661 | 10.842 | −13.203 | 17.247 | 1.00 | 11.06 | A | S |
| ATOM | 3966 | C | CYS | A | 661 | 12.729 | −13.350 | 13.538 | 1.00 | 10.43 | A | C |
| ATOM | 3967 | O | CYS | A | 661 | 12.100 | −12.678 | 12.705 | 1.00 | 10.50 | A | O |
| ATOM | 3968 | N | TRP | A | 662 | 14.036 | −13.597 | 13.427 | 1.00 | 9.89 | A | N |
| ATOM | 3970 | CA | TRP | A | 662 | 14.812 | −13.171 | 12.269 | 1.00 | 9.87 | A | C |
| ATOM | 3972 | CB | TRP | A | 662 | 16.154 | −12.573 | 12.706 | 1.00 | 9.96 | A | C |
| ATOM | 3975 | CG | TRP | A | 662 | 16.045 | −11.379 | 13.624 | 1.00 | 7.99 | A | C |
| ATOM | 3976 | CD1 | TRP | A | 662 | 15.098 | −10.395 | 13.601 | 1.00 | 9.64 | A | C |
| ATOM | 3978 | NE1 | TRP | A | 662 | 15.341 | −9.479 | 14.599 | 1.00 | 9.65 | A | N |
| ATOM | 3980 | CE2 | TRP | A | 662 | 16.458 | −9.867 | 15.292 | 1.00 | 9.01 | A | C |
| ATOM | 3981 | CD2 | TRP | A | 662 | 16.915 | −11.064 | 14.707 | 1.00 | 8.52 | A | C |
| ATOM | 3982 | CE3 | TRP | A | 662 | 18.060 | −11.668 | 15.241 | 1.00 | 8.33 | A | C |
| ATOM | 3984 | CZ3 | TRP | A | 662 | 18.678 | −11.079 | 16.328 | 1.00 | 9.97 | A | C |
| ATOM | 3986 | CH2 | TRP | A | 662 | 18.197 | −9.903 | 16.882 | 1.00 | 9.90 | A | C |
| ATOM | 3988 | CZ2 | TRP | A | 662 | 17.091 | −9.285 | 16.390 | 1.00 | 8.65 | A | C |
| ATOM | 3990 | C | TRP | A | 662 | 15.035 | −14.293 | 11.250 | 1.00 | 10.88 | A | C |
| ATOM | 3991 | O | TRP | A | 662 | 16.003 | −14.279 | 10.518 | 1.00 | 11.46 | A | O |
| ATOM | 3992 | N | ASP | A | 663 | 14.117 | −15.247 | 11.169 | 1.00 | 12.36 | A | N |
| ATOM | 3994 | CA | ASP | A | 663 | 14.145 | −16.205 | 10.079 | 1.00 | 13.31 | A | C |
| ATOM | 3996 | CB | ASP | A | 663 | 13.079 | −17.269 | 10.250 | 1.00 | 13.47 | A | C |
| ATOM | 3999 | CG | ASP | A | 663 | 13.383 | −18.523 | 9.450 | 1.00 | 17.38 | A | C |
| ATOM | 4000 | OD1 | ASP | A | 663 | 13.368 | −18.472 | 8.199 | 1.00 | 19.87 | A | O |
| ATOM | 4001 | OD2 | ASP | A | 663 | 13.623 | −19.611 | 10.004 | 1.00 | 19.38 | A | O |
| ATOM | 4002 | C | ASP | A | 663 | 13.902 | −15.428 | 8.791 | 1.00 | 13.36 | A | C |
| ATOM | 4003 | O | ASP | A | 663 | 13.021 | −14.564 | 8.740 | 1.00 | 12.27 | A | O |
| ATOM | 4004 | N | TYR | A | 664 | 14.708 | −15.693 | 7.772 | 1.00 | 13.88 | A | N |
| ATOM | 4006 | CA | TYR | A | 664 | 14.563 | −14.999 | 6.493 | 1.00 | 14.75 | A | C |
| ATOM | 4008 | CB | TYR | A | 664 | 15.586 | −15.465 | 5.445 | 1.00 | 15.39 | A | C |
| ATOM | 4011 | CG | TYR | A | 664 | 15.757 | −14.437 | 4.351 | 1.00 | 16.86 | A | C |
| ATOM | 4012 | CD1 | TYR | A | 664 | 16.650 | −13.378 | 4.508 | 1.00 | 19.69 | A | C |
| ATOM | 4014 | CE1 | TYR | A | 664 | 16.807 | −12.418 | 3.526 | 1.00 | 21.35 | A | C |
| ATOM | 4016 | CZ | TYR | A | 664 | 16.055 | −12.492 | 2.381 | 1.00 | 21.53 | A | C |
| ATOM | 4017 | OH | TYR | A | 664 | 16.230 | −11.512 | 1.432 | 1.00 | 26.28 | A | O |
| ATOM | 4019 | CE2 | TYR | A | 664 | 15.146 | −13.520 | 2.189 | 1.00 | 21.81 | A | C |
| ATOM | 4021 | CD2 | TYR | A | 664 | 14.992 | −14.490 | 3.181 | 1.00 | 19.31 | A | C |
| ATOM | 4023 | C | TYR | A | 664 | 13.158 | −15.162 | 5.918 | 1.00 | 15.14 | A | C |
| ATOM | 4024 | O | TYR | A | 664 | 12.615 | −14.215 | 5.389 | 1.00 | 15.70 | A | O |
| ATOM | 4025 | N | ASP | A | 665 | 12.602 | −16.366 | 6.020 | 1.00 | 15.67 | A | N |
| ATOM | 4027 | CA | ASP | A | 665 | 11.266 | −16.674 | 5.507 | 1.00 | 16.37 | A | C |
| ATOM | 4029 | CB | ASP | A | 665 | 11.137 | −18.185 | 5.297 | 1.00 | 16.98 | A | C |
| ATOM | 4032 | CG | ASP | A | 665 | 9.894 | −18.570 | 4.536 | 1.00 | 20.27 | A | C |
| ATOM | 4033 | OD1 | ASP | A | 665 | 8.880 | −17.841 | 4.603 | 1.00 | 21.22 | A | O |
| ATOM | 4034 | OD2 | ASP | A | 665 | 9.847 | −19.600 | 3.828 | 1.00 | 25.29 | A | O |
| ATOM | 4035 | C | ASP | A | 665 | 10.197 | −16.222 | 6.508 | 1.00 | 15.72 | A | C |
| ATOM | 4036 | O | ASP | A | 665 | 10.133 | −16.769 | 7.607 | 1.00 | 14.51 | A | O |
| ATOM | 4037 | N | PRO | A | 666 | 9.382 | −15.230 | 6.167 | 1.00 | 16.24 | A | N |
| ATOM | 4038 | CA | PRO | A | 666 | 8.375 | −14.747 | 7.129 | 1.00 | 16.93 | A | C |
| ATOM | 4040 | CB | PRO | A | 666 | 7.583 | −13.690 | 6.346 | 1.00 | 17.41 | A | C |
| ATOM | 4043 | CG | PRO | A | 666 | 8.026 | −13.812 | 4.904 | 1.00 | 17.55 | A | C |
| ATOM | 4046 | CD | PRO | A | 666 | 9.371 | −14.460 | 4.914 | 1.00 | 16.23 | A | C |
| ATOM | 4049 | C | PRO | A | 666 | 7.471 | −15.869 | 7.673 | 1.00 | 17.49 | A | C |
| ATOM | 4050 | O | PRO | A | 666 | 7.117 | −15.846 | 8.857 | 1.00 | 16.21 | A | O |
| ATOM | 4051 | N | SER | A | 667 | 7.161 | −16.855 | 6.823 | 1.00 | 18.52 | A | N |
| ATOM | 4053 | CA | SER | A | 667 | 6.277 | −17.980 | 7.182 | 1.00 | 19.22 | A | C |
| ATOM | 4055 | CB | SER | A | 667 | 6.089 | −18.936 | 5.988 | 1.00 | 19.21 | A | C |
| ATOM | 4058 | OG | SER | A | 667 | 5.265 | −18.349 | 5.018 | 1.00 | 22.33 | A | O |
| ATOM | 4060 | C | SER | A | 667 | 6.774 | −18.786 | 8.355 | 1.00 | 18.58 | A | C |
| ATOM | 4061 | O | SER | A | 667 | 5.977 | −19.427 | 9.028 | 1.00 | 19.88 | A | O |
| ATOM | 4062 | N | ASP | A | 668 | 8.083 | −18.767 | 8.602 | 1.00 | 18.14 | A | N |
| ATOM | 4064 | CA | ASP | A | 668 | 8.691 | −19.514 | 9.690 | 1.00 | 17.77 | A | C |
| ATOM | 4066 | CB | ASP | A | 668 | 10.041 | −20.074 | 9.239 | 1.00 | 18.75 | A | C |
| ATOM | 4069 | CG | ASP | A | 668 | 9.875 | −21.107 | 8.141 | 1.00 | 22.03 | A | C |
| ATOM | 4070 | OD1 | ASP | A | 668 | 10.883 | −21.598 | 7.597 | 1.00 | 27.40 | A | O |
| ATOM | 4071 | OD2 | ASP | A | 668 | 8.741 | −21.464 | 7.754 | 1.00 | 25.19 | A | O |
| ATOM | 4072 | C | ASP | A | 668 | 8.866 | −18.773 | 10.999 | 1.00 | 16.19 | A | C |
| ATOM | 4073 | O | ASP | A | 668 | 9.285 | −19.368 | 11.975 | 1.00 | 16.42 | A | O |
| ATOM | 4074 | N | ARG | A | 669 | 8.544 | −17.484 | 11.041 | 1.00 | 14.15 | A | N |
| ATOM | 4076 | CA | ARG | A | 669 | 8.698 | −16.742 | 12.294 | 1.00 | 12.17 | A | C |
| ATOM | 4078 | CB | ARG | A | 669 | 8.754 | −15.241 | 12.032 | 1.00 | 11.55 | A | C |
| ATOM | 4081 | CG | ARG | A | 669 | 9.876 | −14.823 | 11.139 | 1.00 | 10.93 | A | C |
| ATOM | 4084 | CD | ARG | A | 669 | 9.765 | −13.404 | 10.667 | 1.00 | 8.98 | A | C |
| ATOM | 4087 | NE | ARG | A | 669 | 10.697 | −13.209 | 9.551 | 1.00 | 9.01 | A | N |
| ATOM | 4089 | CZ | ARG | A | 669 | 10.533 | −12.298 | 8.608 | 1.00 | 8.74 | A | C |
| ATOM | 4090 | NH1 | ARG | A | 669 | 9.534 | −11.433 | 8.687 | 1.00 | 9.38 | A | N |
| ATOM | 4093 | NH2 | ARG | A | 669 | 11.399 | −12.224 | 7.601 | 1.00 | 9.94 | A | N |
| ATOM | 4096 | C | ARG | A | 669 | 7.528 | −17.054 | 13.223 | 1.00 | 11.87 | A | C |
| ATOM | 4097 | O | ARG | A | 669 | 6.428 | −17.374 | 12.748 | 1.00 | 11.94 | A | O |
| ATOM | 4098 | N | PRO | A | 670 | 7.721 | −16.936 | 14.531 | 1.00 | 11.24 | A | N |
| ATOM | 4099 | CA | PRO | A | 670 | 6.624 | −17.164 | 15.492 | 1.00 | 10.97 | A | C |
| ATOM | 4101 | CB | PRO | A | 670 | 7.294 | −16.965 | 16.857 | 1.00 | 12.04 | A | C |
| ATOM | 4104 | CG | PRO | A | 670 | 8.752 | −16.960 | 16.597 | 1.00 | 12.70 | A | C |
| ATOM | 4107 | CD | PRO | A | 670 | 8.967 | −16.549 | 15.200 | 1.00 | 11.74 | A | C |
| ATOM | 4110 | C | PRO | A | 670 | 5.502 | −16.141 | 15.365 | 1.00 | 10.78 | A | C |
| ATOM | 4111 | O | PRO | A | 670 | 5.748 | −15.057 | 14.862 | 1.00 | 10.11 | A | O |
| ATOM | 4112 | N | ARG | A | 671 | 4.306 | −16.486 | 15.841 | 1.00 | 10.15 | A | N |
| ATOM | 4114 | CA | ARG | A | 671 | 3.225 | −15.531 | 16.041 | 1.00 | 10.66 | A | C |
| ATOM | 4116 | CB | ARG | A | 671 | 1.913 | −16.274 | 16.265 | 1.00 | 11.28 | A | C |
| ATOM | 4119 | CG | ARG | A | 671 | 1.425 | −17.188 | 15.162 | 1.00 | 14.40 | A | C |
| ATOM | 4122 | CD | ARG | A | 671 | 0.097 | −17.817 | 15.566 | 1.00 | 18.14 | A | C |
| ATOM | 4125 | NE | ARG | A | 671 | −0.602 | −18.573 | 14.531 | 1.00 | 19.61 | A | N |
| ATOM | 4127 | CZ | ARG | A | 671 | −0.460 | −19.884 | 14.309 | 1.00 | 22.54 | A | C |
| ATOM | 4128 | NH1 | ARG | A | 671 | 0.379 | −20.617 | 15.035 | 1.00 | 21.69 | A | N |
| ATOM | 4131 | NH2 | ARG | A | 671 | −1.180 | −20.473 | 13.358 | 1.00 | 23.16 | A | N |
| ATOM | 4134 | C | ARG | A | 671 | 3.478 | −14.710 | 17.304 | 1.00 | 9.70 | A | C |
| ATOM | 4135 | O | ARG | A | 671 | 4.179 | −15.161 | 18.193 | 1.00 | 9.46 | A | O |
| ATOM | 4136 | N | PHE | A | 672 | 2.854 | −13.549 | 17.425 | 1.00 | 9.68 | A | N |
| ATOM | 4138 | CA | PHE | A | 672 | 2.954 | −12.793 | 18.661 | 1.00 | 9.83 | A | C |
| ATOM | 4140 | CB | PHE | A | 672 | 2.376 | −11.377 | 18.529 | 1.00 | 9.02 | A | C |
| ATOM | 4143 | CG | PHE | A | 672 | 3.314 | −10.400 | 17.874 | 1.00 | 7.29 | A | C |
| ATOM | 4144 | CD1 | PHE | A | 672 | 4.499 | −10.034 | 18.499 | 1.00 | 7.29 | A | C |
| ATOM | 4146 | CE1 | PHE | A | 672 | 5.348 | −9.114 | 17.899 | 1.00 | 6.39 | A | C |
| ATOM | 4148 | CZ | PHE | A | 672 | 5.024 | −8.548 | 16.678 | 1.00 | 6.43 | A | C |
| ATOM | 4150 | CE2 | PHE | A | 672 | 3.873 | −8.906 | 16.040 | 1.00 | 7.92 | A | C |
| ATOM | 4152 | CD2 | PHE | A | 672 | 3.008 | −9.841 | 16.639 | 1.00 | 7.47 | A | C |
| ATOM | 4154 | C | PHE | A | 672 | 2.316 | −13.516 | 19.857 | 1.00 | 10.52 | A | C |
| ATOM | 4155 | O | PHE | A | 672 | 2.809 | −13.376 | 20.963 | 1.00 | 10.42 | A | O |
| ATOM | 4156 | N | THR | A | 673 | 1.245 | −14.285 | 19.652 | 1.00 | 11.02 | A | N |
| ATOM | 4158 | CA | THR | A | 673 | 0.664 | −15.049 | 20.764 | 1.00 | 12.24 | A | C |
| ATOM | 4160 | CB | THR | A | 673 | −0.597 | −15.834 | 20.351 | 1.00 | 12.82 | A | C |
| ATOM | 4162 | OG1 | THR | A | 673 | −0.322 | −16.642 | 19.189 | 1.00 | 13.06 | A | O |
| ATOM | 4164 | CG2 | THR | A | 673 | −1.684 | −14.908 | 19.961 | 1.00 | 14.88 | A | C |
| ATOM | 4168 | C | THR | A | 673 | 1.682 | −16.025 | 21.330 | 1.00 | 12.24 | A | C |
| ATOM | 4169 | O | THR | A | 673 | 1.768 | −16.205 | 22.552 | 1.00 | 13.08 | A | O |
| ATOM | 4170 | N | GLU | A | 674 | 2.448 | −16.653 | 20.435 | 1.00 | 11.03 | A | N |
| ATOM | 4172 | CA | GLU | A | 674 | 3.482 | −17.598 | 20.811 | 1.00 | 11.45 | A | C |
| ATOM | 4174 | CB | GLU | A | 674 | 3.971 | −18.366 | 19.575 | 1.00 | 11.48 | A | C |
| ATOM | 4177 | CG | GLU | A | 674 | 2.851 | −19.201 | 18.944 | 1.00 | 12.48 | A | C |
| ATOM | 4180 | CD | GLU | A | 674 | 3.143 | −19.750 | 17.549 | 1.00 | 17.12 | A | C |
| ATOM | 4181 | OE1 | GLU | A | 674 | 4.096 | −19.296 | 16.881 | 1.00 | 15.46 | A | O |
| ATOM | 4182 | OE2 | GLU | A | 674 | 2.370 | −20.657 | 17.120 | 1.00 | 18.19 | A | O |
| ATOM | 4183 | C | GLU | A | 674 | 4.651 | −16.904 | 21.527 | 1.00 | 11.40 | A | C |
| ATOM | 4184 | O | GLU | A | 674 | 5.194 | −17.429 | 22.504 | 1.00 | 12.34 | A | O |
| ATOM | 4185 | N | LEU | A | 675 | 5.006 | −15.714 | 21.070 | 1.00 | 11.03 | A | N |
| ATOM | 4187 | CA | LEU | A | 675 | 6.064 | −14.951 | 21.712 | 1.00 | 10.62 | A | C |
| ATOM | 4189 | CB | LEU | A | 675 | 6.459 | −13.750 | 20.855 | 1.00 | 10.87 | A | C |
| ATOM | 4192 | CG | LEU | A | 675 | 7.212 | −14.093 | 19.574 | 1.00 | 11.12 | A | C |
| ATOM | 4194 | CD1 | LEU | A | 675 | 7.279 | −12.885 | 18.652 | 1.00 | 11.98 | A | C |
| ATOM | 4198 | CD2 | LEU | A | 675 | 8.608 | −14.669 | 19.883 | 1.00 | 12.08 | A | C |
| ATOM | 4202 | C | LEU | A | 675 | 5.674 | −14.489 | 23.125 | 1.00 | 10.64 | A | C |
| ATOM | 4203 | O | LEU | A | 675 | 6.537 | −14.412 | 23.997 | 1.00 | 10.13 | A | O |
| ATOM | 4204 | N | VAL | A | 676 | 4.401 | −14.147 | 23.340 | 1.00 | 10.75 | A | N |
| ATOM | 4206 | CA | VAL | A | 676 | 3.949 | −13.746 | 24.663 | 1.00 | 10.97 | A | C |
| ATOM | 4208 | CB | VAL | A | 676 | 2.481 | −13.353 | 24.703 | 1.00 | 11.26 | A | C |
| ATOM | 4210 | CG1 | VAL | A | 676 | 1.972 | −13.247 | 26.176 | 1.00 | 11.67 | A | C |
| ATOM | 4214 | CG2 | VAL | A | 676 | 2.265 | −12.044 | 23.975 | 1.00 | 11.71 | A | C |
| ATOM | 4218 | C | VAL | A | 676 | 4.193 | −14.922 | 25.623 | 1.00 | 12.06 | A | C |
| ATOM | 4219 | O | VAL | A | 676 | 4.724 | −14.750 | 26.719 | 1.00 | 11.23 | A | O |
| ATOM | 4220 | N | CYS | A | 677 | 3.827 | −16.118 | 25.189 | 1.00 | 12.77 | A | N |
| ATOM | 4222 | CA | CYS | A | 677 | 4.070 | −17.309 | 26.003 | 1.00 | 13.59 | A | C |
| ATOM | 4224 | CB | CYS | A | 677 | 3.486 | −18.526 | 25.322 | 1.00 | 14.78 | A | C |
| ATOM | 4227 | SG | CYS | A | 677 | 1.727 | −18.420 | 25.306 | 1.00 | 23.73 | A | S |
| ATOM | 4228 | C | CYS | A | 677 | 5.544 | −17.543 | 26.310 | 1.00 | 12.70 | A | C |
| ATOM | 4229 | O | CYS | A | 677 | 5.911 | −17.813 | 27.472 | 1.00 | 12.44 | A | O |
| ATOM | 4230 | N | SER | A | 678 | 6.384 | −17.449 | 25.280 | 1.00 | 10.95 | A | N |
| ATOM | 4232 | CA | SER | A | 678 | 7.809 | −17.673 | 25.412 | 1.00 | 11.22 | A | C |
| ATOM | 4234 | CB | SER | A | 678 | 8.501 | −17.631 | 24.052 | 1.00 | 11.49 | A | C |
| ATOM | 4237 | OG | SER | A | 678 | 8.087 | −18.690 | 23.218 | 1.00 | 12.71 | A | O |
| ATOM | 4239 | C | SER | A | 678 | 8.463 | −16.617 | 26.319 | 1.00 | 10.73 | A | C |
| ATOM | 4240 | O | SER | A | 678 | 9.291 | −16.946 | 27.175 | 1.00 | 10.09 | A | O |
| ATOM | 4241 | N | LEU | A | 679 | 8.075 | −15.359 | 26.131 | 1.00 | 10.12 | A | N |
| ATOM | 4243 | CA | LEU | A | 679 | 8.633 | −14.277 | 26.941 | 1.00 | 10.19 | A | C |
| ATOM | 4245 | CB | LEU | A | 679 | 8.272 | −12.920 | 26.386 | 1.00 | 10.05 | A | C |
| ATOM | 4248 | CG | LEU | A | 679 | 9.120 | −12.519 | 25.180 | 1.00 | 10.35 | A | C |
| ATOM | 4250 | CD1 | LEU | A | 679 | 8.594 | −11.187 | 24.639 | 1.00 | 10.06 | A | C |
| ATOM | 4254 | CD2 | LEU | A | 679 | 10.583 | −12.406 | 25.546 | 1.00 | 13.61 | A | C |
| ATOM | 4258 | C | LEU | A | 679 | 8.185 | −14.379 | 28.400 | 1.00 | 10.85 | A | C |
| ATOM | 4259 | O | LEU | A | 679 | 8.975 | −14.105 | 29.293 | 1.00 | 10.86 | A | O |
| ATOM | 4260 | N | SER | A | 680 | 6.944 | −14.812 | 28.632 | 1.00 | 11.33 | A | N |
| ATOM | 4262 | CA | SER | A | 680 | 6.442 | −15.009 | 29.976 | 1.00 | 11.30 | A | C |
| ATOM | 4264 | CB | SER | A | 680 | 4.975 | −15.458 | 29.955 | 1.00 | 11.82 | A | C |
| ATOM | 4267 | OG | SER | A | 680 | 4.160 | −14.393 | 29.529 | 1.00 | 13.70 | A | O |
| ATOM | 4269 | C | SER | A | 680 | 7.294 | −16.054 | 30.696 | 1.00 | 11.10 | A | C |
| ATOM | 4270 | O | SER | A | 680 | 7.569 | −15.914 | 31.876 | 1.00 | 10.06 | A | O |
| ATOM | 4271 | N | ASP | A | 681 | 7.709 | −17.079 | 29.965 | 1.00 | 11.27 | A | N |
| ATOM | 4273 | CA | ASP | A | 681 | 8.555 | −18.151 | 30.489 | 1.00 | 11.79 | A | C |
| ATOM | 4275 | CB | ASP | A | 681 | 8.582 | −19.302 | 29.480 | 1.00 | 12.73 | A | C |
| ATOM | 4278 | CG | ASP | A | 681 | 9.205 | −20.567 | 30.017 | 1.00 | 16.09 | A | C |
| ATOM | 4279 | OD1 | ASP | A | 681 | 9.063 | −20.869 | 31.226 | 1.00 | 18.24 | A | O |
| ATOM | 4280 | OD2 | ASP | A | 681 | 9.823 | −21.351 | 29.260 | 1.00 | 19.49 | A | O |
| ATOM | 4281 | C | ASP | A | 681 | 9.962 | −17.637 | 30.793 | 1.00 | 11.36 | A | C |
| ATOM | 4282 | O | ASP | A | 681 | 10.524 | −17.962 | 31.832 | 1.00 | 10.57 | A | O |
| ATOM | 4283 | N | VAL | A | 682 | 10.514 | −16.805 | 29.911 | 1.00 | 10.17 | A | N |
| ATOM | 4285 | CA | VAL | A | 682 | 11.831 | −16.189 | 30.137 | 1.00 | 10.71 | A | C |
| ATOM | 4287 | CB | VAL | A | 682 | 12.323 | −15.421 | 28.879 | 1.00 | 11.06 | A | C |
| ATOM | 4289 | CG1 | VAL | A | 682 | 13.585 | −14.610 | 29.186 | 1.00 | 13.37 | A | C |
| ATOM | 4293 | CG2 | VAL | A | 682 | 12.586 | −16.394 | 27.719 | 1.00 | 12.19 | A | C |
| ATOM | 4297 | C | VAL | A | 682 | 11.784 | −15.270 | 31.371 | 1.00 | 10.10 | A | C |
| ATOM | 4298 | O | VAL | A | 682 | 12.698 | −15.271 | 32.194 | 1.00 | 9.87 | A | O |
| ATOM | 4299 | N | TYR | A | 683 | 10.713 | −14.501 | 31.498 | 1.00 | 9.76 | A | N |
| ATOM | 4301 | CA | TYR | A | 683 | 10.535 | −13.588 | 32.615 | 1.00 | 10.41 | A | C |
| ATOM | 4303 | CB | TYR | A | 683 | 9.258 | −12.801 | 32.428 | 1.00 | 10.16 | A | C |
| ATOM | 4306 | CG | TYR | A | 683 | 8.986 | −11.749 | 33.480 | 1.00 | 11.77 | A | C |
| ATOM | 4307 | CD1 | TYR | A | 683 | 9.973 | −10.837 | 33.858 | 1.00 | 12.15 | A | C |
| ATOM | 4309 | CE1 | TYR | A | 683 | 9.719 | −9.862 | 34.816 | 1.00 | 14.98 | A | C |
| ATOM | 4311 | CZ | TYR | A | 683 | 8.480 | −9.787 | 35.386 | 1.00 | 15.15 | A | C |
| ATOM | 4312 | OH | TYR | A | 683 | 8.225 | −8.818 | 36.312 | 1.00 | 16.29 | A | O |
| ATOM | 4314 | CE2 | TYR | A | 683 | 7.477 | −10.669 | 35.022 | 1.00 | 16.42 | A | C |
| ATOM | 4316 | CD2 | TYR | A | 683 | 7.743 | −11.648 | 34.070 | 1.00 | 13.80 | A | C |
| ATOM | 4318 | C | TYR | A | 683 | 10.482 | −14.356 | 33.933 | 1.00 | 10.77 | A | C |
| ATOM | 4319 | O | TYR | A | 683 | 11.155 | −13.994 | 34.896 | 1.00 | 10.66 | A | O |
| ATOM | 4320 | N | GLN | A | 684 | 9.701 | −15.438 | 33.952 | 1.00 | 11.00 | A | N |
| ATOM | 4322 | CA | GLN | A | 684 | 9.618 | −16.300 | 35.132 | 1.00 | 11.22 | A | C |
| ATOM | 4324 | CB | GLN | A | 684 | 8.629 | −17.444 | 34.930 | 1.00 | 11.31 | A | C |
| ATOM | 4327 | CG | GLN | A | 684 | 8.375 | −18.269 | 36.236 | 1.00 | 13.65 | A | C |
| ATOM | 4330 | CD | GLN | A | 684 | 7.820 | −17.402 | 37.374 | 1.00 | 15.94 | A | C |
| ATOM | 4331 | OE1 | GLN | A | 684 | 8.477 | −17.197 | 38.403 | 1.00 | 19.21 | A | O |
| ATOM | 4332 | NE2 | GLN | A | 684 | 6.630 | −16.887 | 37.180 | 1.00 | 16.15 | A | N |
| ATOM | 4335 | C | GLN | A | 684 | 10.971 | −16.890 | 35.468 | 1.00 | 11.59 | A | C |
| ATOM | 4336 | O | GLN | A | 684 | 11.344 | −16.943 | 36.636 | 1.00 | 11.70 | A | O |
| ATOM | 4337 | N | MET | A | 685 | 11.690 | −17.356 | 34.453 | 1.00 | 12.42 | A | N |
| ATOM | 4339 | CA | MET | A | 685 | 13.022 | −17.913 | 34.646 | 1.00 | 14.85 | A | C |
| ATOM | 4341 | CB | MET | A | 685 | 13.616 | −18.457 | 33.334 | 1.00 | 15.51 | A | C |
| ATOM | 4344 | CG | MET | A | 685 | 15.075 | −18.894 | 33.413 | 1.00 | 20.31 | A | C |
| ATOM | 4347 | SD | MET | A | 685 | 16.347 | −17.557 | 33.428 | 1.00 | 30.38 | A | S |
| ATOM | 4348 | CE | MET | A | 685 | 16.971 | −17.643 | 31.702 | 1.00 | 29.94 | A | C |
| ATOM | 4352 | C | MET | A | 685 | 13.952 | −16.870 | 35.251 | 1.00 | 14.79 | A | C |
| ATOM | 4353 | O | MET | A | 685 | 14.707 | −17.201 | 36.149 | 1.00 | 14.67 | A | O |
| ATOM | 4354 | N | GLU | A | 686 | 13.908 | −15.623 | 34.767 | 1.00 | 15.09 | A | N |
| ATOM | 4356 | CA | GLU | A | 686 | 14.782 | −14.564 | 35.321 | 1.00 | 15.60 | A | C |
| ATOM | 4358 | CB | GLU | A | 686 | 14.765 | −13.293 | 34.457 | 1.00 | 15.66 | A | C |
| ATOM | 4361 | CG | GLU | A | 686 | 15.520 | −13.394 | 33.143 | 1.00 | 18.58 | A | C |
| ATOM | 4364 | CD | GLU | A | 686 | 17.018 | −13.650 | 33.305 | 1.00 | 19.75 | A | C |
| ATOM | 4365 | OE1 | GLU | A | 686 | 17.692 | −12.967 | 34.110 | 1.00 | 19.12 | A | O |
| ATOM | 4366 | OE2 | GLU | A | 686 | 17.521 | −14.545 | 32.603 | 1.00 | 25.71 | A | O |
| ATOM | 4367 | C | GLU | A | 686 | 14.431 | −14.212 | 36.776 | 1.00 | 15.84 | A | C |
| ATOM | 4368 | O | GLU | A | 686 | 15.319 | −13.886 | 37.568 | 1.00 | 15.94 | A | O |
| ATOM | 4369 | N | LYS | A | 687 | 13.146 | −14.251 | 37.124 | 1.00 | 15.72 | A | N |
| ATOM | 4371 | CA | LYS | A | 687 | 12.727 | −14.031 | 38.499 | 1.00 | 16.97 | A | C |
| ATOM | 4373 | CB | LYS | A | 687 | 11.206 | −13.895 | 38.594 | 1.00 | 16.88 | A | C |
| ATOM | 4376 | CG | LYS | A | 687 | 10.707 | −12.578 | 38.036 | 1.00 | 17.92 | A | C |
| ATOM | 4379 | CD | LYS | A | 687 | 9.303 | −12.243 | 38.452 | 1.00 | 19.62 | A | C |
| ATOM | 4382 | CE | LYS | A | 687 | 8.270 | −13.060 | 37.692 | 1.00 | 20.81 | A | C |
| ATOM | 4385 | NZ | LYS | A | 687 | 6.878 | −12.646 | 38.085 | 1.00 | 20.18 | A | N |
| ATOM | 4389 | C | LYS | A | 687 | 13.225 | −15.171 | 39.407 | 1.00 | 17.45 | A | C |
| ATOM | 4390 | O | LYS | A | 687 | 13.629 | −14.925 | 40.534 | 1.00 | 18.25 | A | O |
| ATOM | 4391 | N | ASP | A | 688 | 13.214 | −16.399 | 38.900 | 1.00 | 18.00 | A | N |
| ATOM | 4393 | CA | ASP | A | 688 | 13.607 | −17.573 | 39.679 | 1.00 | 19.39 | A | C |
| ATOM | 4395 | CB | ASP | A | 688 | 13.129 | −18.859 | 38.991 | 1.00 | 19.60 | A | C |
| ATOM | 4398 | CG | ASP | A | 688 | 11.616 | −19.005 | 39.009 | 1.00 | 20.07 | A | C |
| ATOM | 4399 | OD1 | ASP | A | 688 | 10.939 | −18.210 | 39.683 | 1.00 | 18.55 | A | O |
| ATOM | 4400 | OD2 | ASP | A | 688 | 11.017 | −19.885 | 38.363 | 1.00 | 21.49 | A | O |
| ATOM | 4401 | C | ASP | A | 688 | 15.114 | −17.677 | 39.938 | 1.00 | 20.75 | A | C |
| ATOM | 4402 | O | ASP | A | 688 | 15.519 | −18.280 | 40.936 | 1.00 | 20.21 | A | O |
| ATOM | 4403 | N | ILE | A | 689 | 15.932 | −17.094 | 39.064 | 1.00 | 22.34 | A | N |
| ATOM | 4405 | CA | ILE | A | 689 | 17.401 | −17.136 | 39.206 | 1.00 | 24.85 | A | C |
| ATOM | 4407 | CB | ILE | A | 689 | 18.096 | −17.337 | 37.831 | 1.00 | 25.22 | A | C |
| ATOM | 4409 | CG1 | ILE | A | 689 | 18.027 | −16.062 | 36.982 | 1.00 | 24.97 | A | C |
| ATOM | 4412 | CD1 | ILE | A | 689 | 18.969 | −16.063 | 35.816 | 1.00 | 26.04 | A | C |
| ATOM | 4416 | CG2 | ILE | A | 689 | 17.485 | −18.515 | 37.100 | 1.00 | 26.30 | A | C |
| ATOM | 4420 | C | ILE | A | 689 | 17.989 | −15.894 | 39.845 | 1.00 | 26.98 | A | C |
| ATOM | 4421 | O | ILE | A | 689 | 19.220 | −15.772 | 39.945 | 1.00 | 27.14 | A | O |
| ATOM | 4422 | N | ALA | A | 690 | 17.122 | −14.964 | 40.247 | 1.00 | 29.52 | A | N |
| ATOM | 4424 | CA | ALA | A | 690 | 17.543 | −13.708 | 40.840 | 1.00 | 31.03 | A | C |
| ATOM | 4426 | CB | ALA | A | 690 | 16.576 | −12.605 | 40.460 | 1.00 | 31.13 | A | C |
| ATOM | 4430 | C | ALA | A | 690 | 17.629 | −13.834 | 42.357 | 1.00 | 32.63 | A | C |
| ATOM | 4431 | O | ALA | A | 690 | 16.599 | −13.900 | 43.042 | 1.00 | 32.75 | A | O |
| ATOM | 4432 | N | MET | A | 691 | 18.863 | −13.859 | 42.864 | 1.00 | 34.71 | A | N |
| ATOM | 4434 | CA | MET | A | 691 | 19.159 | −13.824 | 44.304 | 1.00 | 35.61 | A | C |
| ATOM | 4436 | CB | MET | A | 691 | 18.556 | −12.572 | 44.955 | 1.00 | 36.56 | A | C |
| ATOM | 4439 | CG | MET | A | 691 | 19.081 | −11.260 | 44.382 | 1.00 | 39.02 | A | C |
| ATOM | 4442 | SD | MET | A | 691 | 18.165 | −9.844 | 45.010 | 1.00 | 44.70 | A | S |
| ATOM | 4443 | CE | MET | A | 691 | 16.654 | −10.005 | 44.062 | 1.00 | 44.31 | A | C |
| ATOM | 4447 | C | MET | A | 691 | 18.685 | −15.085 | 45.012 | 1.00 | 35.54 | A | C |
| ATOM | 4448 | O | MET | A | 691 | 18.582 | −16.116 | 44.329 | 1.00 | 35.63 | A | O |
| ATOM | 4449 | OXT | MET | A | 691 | 18.437 | −15.031 | 46.228 | 1.00 | 34.84 | A | O |
| ATOM | 4450 | O1A | ANP | L | 1 | 5.879 | 14.129 | 17.474 | 1.00 | 56.03 | L | O |
| ATOM | 4451 | PA | ANP | L | 1 | 5.828 | 13.237 | 18.779 | 1.00 | 54.99 | L | P |
| ATOM | 4452 | O2A | ANP | L | 1 | 4.359 | 12.664 | 18.974 | 1.00 | 55.47 | L | O |
| ATOM | 4454 | O3A | ANP | L | 1 | 6.928 | 12.057 | 18.803 | 1.00 | 58.70 | L | O |
| ATOM | 4455 | PB | ANP | L | 1 | 7.534 | 11.319 | 17.499 | 1.00 | 61.85 | L | P |
| ATOM | 4456 | O1B | ANP | L | 1 | 6.806 | 11.789 | 16.164 | 1.00 | 60.90 | L | O |
| ATOM | 4457 | O2B | ANP | L | 1 | 7.338 | 9.749 | 17.688 | 1.00 | 61.90 | L | O |
| ATOM | 4459 | N3B | ANP | L | 1 | 9.254 | 11.643 | 17.401 | 1.00 | 63.51 | L | N |
| ATOM | 4461 | PG | ANP | L | 1 | 9.852 | 13.230 | 16.957 | 1.00 | 65.53 | L | P |
| ATOM | 4462 | O3G | ANP | L | 1 | 10.884 | 13.100 | 15.748 | 1.00 | 66.11 | L | O |
| ATOM | 4464 | O2G | ANP | L | 1 | 10.586 | 13.911 | 18.193 | 1.00 | 64.86 | L | O |
| ATOM | 4466 | O1G | ANP | L | 1 | 8.643 | 14.135 | 16.458 | 1.00 | 65.59 | L | O |
| ATOM | 4467 | O5* | ANP | L | 1 | 6.277 | 14.111 | 20.050 | 1.00 | 51.13 | L | O |
| ATOM | 4468 | C5* | ANP | L | 1 | 7.549 | 14.739 | 20.107 | 1.00 | 46.73 | L | C |
| ATOM | 4471 | C4* | ANP | L | 1 | 8.168 | 14.467 | 21.461 | 1.00 | 44.62 | L | C |
| ATOM | 4473 | O4* | ANP | L | 1 | 7.254 | 14.809 | 22.510 | 1.00 | 41.60 | L | O |
| ATOM | 4474 | C1* | ANP | L | 1 | 7.281 | 13.799 | 23.515 | 1.00 | 38.50 | L | C |
| ATOM | 4476 | C2* | ANP | L | 1 | 8.155 | 12.643 | 23.037 | 1.00 | 42.11 | L | C |
| ATOM | 4478 | O2* | ANP | L | 1 | 9.304 | 12.486 | 23.860 | 1.00 | 44.90 | L | O |
| ATOM | 4480 | C3* | ANP | L | 1 | 8.524 | 12.998 | 21.614 | 1.00 | 43.96 | L | C |
| ATOM | 4482 | O3* | ANP | L | 1 | 9.901 | 12.797 | 21.351 | 1.00 | 45.53 | L | O |
| ATOM | 4484 | N9 | ANP | L | 1 | 5.951 | 13.197 | 23.740 | 1.00 | 31.30 | L | N |
| ATOM | 4485 | C8 | ANP | L | 1 | 5.015 | 12.876 | 22.819 | 1.00 | 28.19 | L | C |
| ATOM | 4487 | N7 | ANP | L | 1 | 3.913 | 12.295 | 23.381 | 1.00 | 25.49 | L | N |
| ATOM | 4488 | C5 | ANP | L | 1 | 4.161 | 12.228 | 24.694 | 1.00 | 24.63 | L | C |
| ATOM | 4489 | C6 | ANP | L | 1 | 3.506 | 11.746 | 25.918 | 1.00 | 21.30 | L | C |
| ATOM | 4490 | N6 | ANP | L | 1 | 2.277 | 11.204 | 25.779 | 1.00 | 18.20 | L | N |
| ATOM | 4493 | C4 | ANP | L | 1 | 5.475 | 12.793 | 24.898 | 1.00 | 26.86 | L | C |
| ATOM | 4494 | N3 | ANP | L | 1 | 6.067 | 12.897 | 26.207 | 1.00 | 23.82 | L | N |
| ATOM | 4495 | C2 | ANP | L | 1 | 5.359 | 12.433 | 27.261 | 1.00 | 22.61 | L | C |
| ATOM | 4497 | N1 | ANP | L | 1 | 4.129 | 11.883 | 27.114 | 1.00 | 19.81 | L | N |
| ATOM | 4498 | O | HOH | M | 1 | 11.906 | 10.877 | 19.048 | 1.00 | 54.80 | M | O |
| ATOM | 4501 | O | HOH | W | 1 | 19.443 | −9.552 | 13.290 | 1.00 | 12.78 | W | O |
| ATOM | 4504 | O | HOH | W | 2 | 24.437 | −5.004 | 22.245 | 1.00 | 16.30 | W | O |
| ATOM | 4507 | O | HOH | W | 3 | 16.956 | −5.546 | 11.116 | 1.00 | 14.32 | W | O |
| ATOM | 4510 | O | HOH | W | 4 | 23.654 | −5.882 | 27.256 | 1.00 | 16.76 | W | O |
| ATOM | 4513 | O | HOH | W | 5 | 19.773 | −2.500 | 2.719 | 1.00 | 17.82 | W | O |
| ATOM | 4516 | O | HOH | W | 6 | 14.677 | 3.342 | 18.254 | 1.00 | 12.86 | W | O |
| ATOM | 4519 | O | HOH | W | 7 | 15.460 | −2.754 | 37.319 | 1.00 | 19.09 | W | O |
| ATOM | 4522 | O | HOH | W | 8 | 0.917 | −6.788 | 13.617 | 1.00 | 19.87 | W | O |
| ATOM | 4525 | O | HOH | W | 9 | 6.262 | −4.291 | 9.880 | 1.00 | 17.35 | W | O |
| ATOM | 4528 | O | HOH | W | 10 | −8.084 | −9.594 | 20.676 | 1.00 | 19.55 | W | O |
| ATOM | 4531 | O | HOH | W | 11 | 9.378 | −6.760 | 37.711 | 1.00 | 23.16 | W | O |
| ATOM | 4534 | O | HOH | W | 12 | −4.324 | 2.362 | 25.030 | 1.00 | 17.21 | W | O |
| ATOM | 4537 | O | HOH | W | 13 | 2.631 | −4.978 | 2.213 | 1.00 | 21.67 | W | O |
| ATOM | 4540 | O | HOH | W | 14 | 13.357 | 9.278 | 28.253 | 1.00 | 18.85 | W | O |
| ATOM | 4543 | O | HOH | W | 15 | 18.009 | 2.532 | 9.354 | 1.00 | 17.12 | W | O |
| ATOM | 4546 | O | HOH | W | 16 | 17.534 | −9.551 | 37.009 | 1.00 | 20.96 | W | O |
| ATOM | 4549 | O | HOH | W | 17 | 27.129 | −8.112 | 21.598 | 1.00 | 20.80 | W | O |
| ATOM | 4552 | O | HOH | W | 18 | 18.189 | −15.606 | 12.347 | 1.00 | 21.79 | W | O |
| ATOM | 4555 | O | HOH | W | 19 | 13.177 | 0.863 | 35.291 | 1.00 | 23.40 | W | O |
| ATOM | 4558 | O | HOH | W | 20 | 17.054 | −4.703 | 8.729 | 1.00 | 17.25 | W | O |
| ATOM | 4561 | O | HOH | W | 21 | 7.866 | −18.942 | 20.500 | 1.00 | 24.06 | W | O |
| ATOM | 4564 | O | HOH | W | 22 | 14.700 | 7.035 | 22.806 | 1.00 | 16.93 | W | O |
| ATOM | 4567 | O | HOH | W | 23 | −2.679 | 2.607 | 28.131 | 1.00 | 23.11 | W | O |
| ATOM | 4570 | O | HOH | W | 24 | 6.040 | −9.803 | 37.824 | 1.00 | 33.92 | W | O |
| ATOM | 4573 | O | HOH | W | 25 | 10.004 | −5.316 | 9.507 | 1.00 | 23.61 | W | O |
| ATOM | 4576 | O | HOH | W | 26 | 25.491 | −5.320 | 24.819 | 1.00 | 23.18 | W | O |
| ATOM | 4579 | O | HOH | W | 27 | 4.534 | 32.921 | 12.514 | 1.00 | 20.02 | W | O |
| ATOM | 4582 | O | HOH | W | 28 | 21.903 | −11.752 | 31.783 | 1.00 | 27.60 | W | O |
| ATOM | 4585 | O | HOH | W | 29 | −2.817 | −12.294 | 18.585 | 1.00 | 19.69 | W | O |
| ATOM | 4588 | O | HOH | W | 30 | 0.619 | 25.243 | 18.899 | 1.00 | 22.14 | W | O |
| ATOM | 4591 | O | HOH | W | 31 | 4.508 | −2.772 | −1.220 | 1.00 | 21.13 | W | O |
| ATOM | 4594 | O | HOH | W | 32 | 26.872 | −11.629 | 24.125 | 1.00 | 24.32 | W | O |
| ATOM | 4597 | O | HOH | W | 33 | 26.063 | −13.556 | 19.248 | 1.00 | 23.37 | W | O |
| ATOM | 4600 | O | HOH | W | 34 | −4.463 | 26.591 | 28.285 | 1.00 | 28.20 | W | O |
| ATOM | 4603 | O | HOH | W | 35 | −0.377 | −9.432 | 8.082 | 1.00 | 25.76 | W | O |
| ATOM | 4606 | O | HOH | W | 36 | 11.357 | 4.915 | 15.323 | 1.00 | 31.17 | W | O |
| ATOM | 4609 | O | HOH | W | 37 | 0.444 | −0.589 | 8.490 | 1.00 | 27.25 | W | O |
| ATOM | 4612 | O | HOH | W | 38 | 12.025 | −12.305 | 3.518 | 1.00 | 20.13 | W | O |
| ATOM | 4615 | O | HOH | W | 39 | −7.592 | 21.532 | 34.079 | 1.00 | 24.16 | W | O |
| ATOM | 4618 | O | HOH | W | 40 | −3.034 | 25.675 | 20.623 | 1.00 | 27.86 | W | O |
| ATOM | 4621 | O | HOH | W | 41 | 19.252 | −18.574 | 29.007 | 1.00 | 24.11 | W | O |
| ATOM | 4624 | O | HOH | W | 42 | 18.447 | 5.050 | 22.545 | 1.00 | 26.98 | W | O |
| ATOM | 4627 | O | HOH | W | 43 | 18.236 | 2.548 | 19.150 | 1.00 | 23.34 | W | O |
| ATOM | 4630 | O | HOH | W | 44 | 23.599 | −4.581 | 0.730 | 1.00 | 23.86 | W | O |
| ATOM | 4633 | O | HOH | W | 45 | 5.670 | −14.733 | 33.566 | 1.00 | 22.95 | W | O |
| ATOM | 4636 | O | HOH | W | 46 | 14.080 | −19.737 | 23.406 | 1.00 | 22.24 | W | O |
| ATOM | 4639 | O | HOH | W | 47 | 22.734 | 4.890 | 34.891 | 1.00 | 26.97 | W | O |
| ATOM | 4642 | O | HOH | W | 48 | −3.602 | 22.418 | 16.052 | 1.00 | 29.22 | W | O |
| ATOM | 4645 | O | HOH | W | 49 | 25.830 | −12.392 | 15.030 | 1.00 | 32.70 | W | O |
| ATOM | 4648 | O | HOH | W | 50 | −2.320 | −15.117 | 16.246 | 1.00 | 28.09 | W | O |
| ATOM | 4651 | O | HOH | W | 51 | 2.614 | −23.096 | 16.430 | 1.00 | 31.67 | W | O |
| ATOM | 4654 | O | HOH | W | 52 | 17.362 | −18.998 | 43.191 | 1.00 | 26.88 | W | O |
| ATOM | 4657 | O | HOH | W | 53 | 26.474 | −8.992 | 12.337 | 1.00 | 31.87 | W | O |
| ATOM | 4660 | O | HOH | W | 54 | 16.830 | −4.474 | −1.805 | 1.00 | 36.41 | W | O |
| ATOM | 4663 | O | HOH | W | 55 | 4.947 | −4.071 | 36.311 | 1.00 | 25.83 | W | O |
| ATOM | 4666 | O | HOH | W | 56 | 1.631 | 1.495 | 10.141 | 1.00 | 25.82 | W | O |
| ATOM | 4669 | O | HOH | W | 57 | 21.157 | −1.727 | −0.971 | 1.00 | 32.21 | W | O |
| ATOM | 4672 | O | HOH | W | 58 | 20.249 | −8.173 | 36.530 | 1.00 | 21.67 | W | O |
| ATOM | 4675 | O | HOH | W | 59 | −2.610 | −9.023 | 9.489 | 1.00 | 35.63 | W | O |
| ATOM | 4678 | O | HOH | W | 60 | −9.404 | 23.395 | 33.556 | 1.00 | 27.23 | W | O |
| ATOM | 4681 | O | HOH | W | 61 | 11.153 | 3.802 | −0.793 | 1.00 | 32.48 | W | O |
| ATOM | 4684 | O | HOH | W | 62 | 2.156 | −13.178 | 30.950 | 1.00 | 32.64 | W | O |
| ATOM | 4687 | O | HOH | W | 63 | 15.393 | −13.901 | 45.580 | 1.00 | 34.94 | W | O |
| ATOM | 4690 | O | HOH | W | 64 | 5.734 | −20.060 | 12.705 | 1.00 | 28.43 | W | O |
| ATOM | 4693 | O | HOH | W | 65 | −0.390 | 18.866 | 1.150 | 1.00 | 37.04 | W | O |
| ATOM | 4696 | O | HOH | W | 66 | 9.308 | 23.821 | 20.537 | 1.00 | 33.19 | W | O |
| ATOM | 4699 | O | HOH | W | 67 | −8.657 | −12.592 | 15.980 | 1.00 | 30.95 | W | O |
| ATOM | 4702 | O | HOH | W | 68 | 6.009 | −10.288 | 5.499 | 1.00 | 30.71 | W | O |
| ATOM | 4705 | O | HOH | W | 69 | 5.849 | −14.630 | 36.206 | 1.00 | 36.64 | W | O |
| ATOM | 4708 | O | HOH | W | 70 | −0.379 | −15.926 | 24.303 | 1.00 | 26.34 | W | O |
| ATOM | 4711 | O | HOH | W | 71 | 19.013 | 6.091 | 37.596 | 1.00 | 29.23 | W | O |
| ATOM | 4714 | O | HOH | W | 72 | −2.437 | −8.935 | 24.621 | 1.00 | 26.00 | W | O |
| ATOM | 4717 | O | HOH | W | 73 | 13.274 | 10.054 | 0.171 | 1.00 | 32.22 | W | O |
| ATOM | 4720 | O | HOH | W | 74 | 11.266 | −4.798 | 37.300 | 1.00 | 27.56 | W | O |
| ATOM | 4723 | O | HOH | W | 75 | 18.634 | 5.650 | 3.509 | 1.00 | 30.22 | W | O |
| ATOM | 4726 | O | HOH | W | 76 | 17.088 | −17.411 | 8.048 | 1.00 | 26.93 | W | O |
| ATOM | 4729 | O | HOH | W | 77 | 23.077 | 3.880 | 2.721 | 1.00 | 25.25 | W | O |
| ATOM | 4732 | O | HOH | W | 78 | 24.631 | 5.289 | 28.100 | 1.00 | 53.16 | W | O |
| ATOM | 4735 | O | HOH | W | 79 | 10.896 | −4.046 | −5.174 | 1.00 | 30.72 | W | O |
| ATOM | 4738 | O | HOH | W | 80 | 21.483 | −18.240 | 22.299 | 1.00 | 31.63 | W | O |
| ATOM | 4741 | O | HOH | W | 81 | 17.664 | −12.362 | 36.744 | 1.00 | 27.21 | W | O |
| ATOM | 4744 | O | HOH | W | 82 | −6.800 | −5.096 | 9.419 | 1.00 | 31.32 | W | O |
| ATOM | 4747 | O | HOH | W | 83 | 3.254 | −6.214 | 33.543 | 1.00 | 29.36 | W | O |
| ATOM | 4750 | O | HOH | W | 84 | −6.655 | 5.297 | 23.427 | 1.00 | 38.97 | W | O |
| ATOM | 4753 | O | HOH | W | 85 | 6.119 | 3.003 | 3.176 | 1.00 | 39.55 | W | O |
| ATOM | 4756 | O | HOH | W | 86 | 16.440 | 5.449 | 7.469 | 1.00 | 35.83 | W | O |
| ATOM | 4759 | O | HOH | W | 87 | −6.743 | 15.088 | 41.223 | 1.00 | 33.66 | W | O |
| ATOM | 4762 | O | HOH | W | 88 | −8.714 | −10.654 | 12.475 | 1.00 | 45.30 | W | O |
| ATOM | 4765 | O | HOH | W | 89 | 5.455 | 6.147 | 14.560 | 1.00 | 34.25 | W | O |
| ATOM | 4768 | O | HOH | W | 90 | 7.863 | −2.178 | 41.320 | 1.00 | 41.21 | W | O |
| ATOM | 4771 | O | HOH | W | 91 | 3.037 | −8.511 | 31.787 | 1.00 | 43.98 | W | O |
| ATOM | 4774 | O | HOH | W | 92 | −5.700 | 2.904 | 27.490 | 1.00 | 30.78 | W | O |
| ATOM | 4777 | O | HOH | W | 93 | 10.698 | −16.094 | 41.240 | 1.00 | 40.42 | W | O |
| ATOM | 4780 | O | HOH | W | 94 | 8.297 | −21.654 | 26.890 | 1.00 | 35.50 | W | O |
| ATOM | 4783 | O | HOH | W | 95 | −7.043 | 26.092 | 27.806 | 1.00 | 33.08 | W | O |
| ATOM | 4786 | O | HOH | W | 96 | 12.953 | −20.340 | 21.075 | 1.00 | 26.90 | W | O |
| ATOM | 4789 | O | HOH | W | 97 | −1.020 | 6.390 | 14.791 | 1.00 | 42.20 | W | O |
| ATOM | 4792 | O | HOH | W | 98 | 9.903 | −2.480 | 37.263 | 1.00 | 39.90 | W | O |
| ATOM | 4795 | O | HOH | W | 99 | −14.690 | 5.403 | 20.304 | 1.00 | 43.77 | W | O |
| ATOM | 4798 | O | HOH | W | 100 | 7.497 | −20.614 | 33.258 | 1.00 | 42.00 | W | O |
| ATOM | 4801 | O | HOH | W | 101 | −6.310 | −0.802 | 28.924 | 1.00 | 38.73 | W | O |
| ATOM | 4804 | O | HOH | W | 102 | 23.429 | 2.695 | 26.110 | 1.00 | 30.05 | W | O |
| ATOM | 4807 | O | HOH | W | 103 | 5.939 | 23.666 | 15.058 | 1.00 | 45.24 | W | O |
| ATOM | 4810 | O | HOH | W | 104 | 15.157 | −8.657 | 41.812 | 1.00 | 48.42 | W | O |
| ATOM | 4813 | O | HOH | W | 105 | 22.584 | 5.351 | 6.685 | 1.00 | 32.02 | W | O |
| ATOM | 4816 | O | HOH | W | 106 | 1.947 | −13.531 | 12.995 | 1.00 | 35.70 | W | O |
| ATOM | 4819 | O | HOH | W | 107 | 0.843 | −3.343 | 5.954 | 1.00 | 35.00 | W | O |
| ATOM | 4822 | O | HOH | W | 108 | 10.764 | 8.978 | −0.078 | 1.00 | 47.97 | W | O |
| ATOM | 4825 | O | HOH | W | 109 | 12.609 | 11.593 | 33.799 | 1.00 | 33.88 | W | O |
| ATOM | 4828 | O | HOH | W | 110 | −2.559 | 28.085 | 29.916 | 1.00 | 40.24 | W | O |
| ATOM | 4831 | O | HOH | W | 111 | −8.695 | −8.532 | 8.837 | 1.00 | 37.60 | W | O |
| ATOM | 4834 | O | HOH | W | 112 | 1.265 | 18.134 | 33.506 | 1.00 | 37.44 | W | O |
| ATOM | 4837 | O | HOH | W | 113 | −9.981 | 6.869 | 11.619 | 1.00 | 38.18 | W | O |
| ATOM | 4840 | O | HOH | W | 114 | −2.744 | 14.784 | 38.949 | 1.00 | 50.37 | W | O |
| ATOM | 4843 | O | HOH | W | 115 | 20.301 | −12.723 | 34.212 | 1.00 | 40.49 | W | O |
| ATOM | 4846 | O | HOH | W | 116 | 14.851 | 9.838 | 20.023 | 1.00 | 37.48 | W | O |
| ATOM | 4849 | O | HOH | W | 117 | 23.651 | 3.698 | 39.134 | 1.00 | 37.78 | W | O |
| ATOM | 4852 | O | HOH | W | 118 | −18.557 | 13.257 | 30.128 | 1.00 | 41.89 | W | O |
| ATOM | 4855 | O | HOH | W | 119 | 18.487 | −21.421 | 23.859 | 1.00 | 39.29 | W | O |
| ATOM | 4858 | O | HOH | W | 120 | 21.377 | 4.874 | 37.522 | 1.00 | 28.87 | W | O |
| ATOM | 4861 | O | HOH | W | 121 | 18.415 | −7.751 | −2.248 | 1.00 | 36.44 | W | O |
| ATOM | 4864 | O | HOH | W | 122 | 17.602 | −7.842 | 12.411 | 1.00 | 16.38 | W | O |
| ATOM | 4867 | O | HOH | W | 123 | 16.678 | 4.872 | 18.751 | 1.00 | 22.46 | W | O |
| ATOM | 4870 | O | HOH | W | 124 | 14.066 | −0.309 | 37.545 | 1.00 | 30.70 | W | O |
| ATOM | 4873 | O | HOH | W | 125 | 7.740 | −4.673 | 37.771 | 1.00 | 27.15 | W | O |
| ATOM | 4876 | O | HOH | W | 126 | 13.728 | 4.405 | 15.630 | 1.00 | 25.76 | W | O |
| ATOM | 4879 | O | HOH | W | 127 | 27.821 | −6.501 | 23.920 | 1.00 | 31.77 | W | O |
| ATOM | 4882 | O | HOH | W | 128 | −3.928 | 27.852 | 25.640 | 1.00 | 29.97 | W | O |
| ATOM | 4885 | O | HOH | W | 129 | 16.702 | 6.462 | 21.069 | 1.00 | 30.52 | W | O |
| ATOM | 4888 | O | HOH | W | 130 | 26.185 | −5.646 | 20.509 | 1.00 | 24.01 | W | O |
| ATOM | 4891 | O | HOH | W | 131 | 1.974 | −3.227 | 0.396 | 1.00 | 29.45 | W | O |
| ATOM | 4894 | O | HOH | W | 132 | 18.503 | −17.071 | 10.260 | 1.00 | 34.39 | W | O |
| ATOM | 4897 | O | HOH | W | 133 | 10.397 | −13.616 | 1.651 | 1.00 | 39.32 | W | O |
| ATOM | 4900 | O | HOH | W | 134 | −2.831 | 28.209 | 21.200 | 1.00 | 30.33 | W | O |
| ATOM | 4903 | O | HOH | W | 135 | 15.320 | −22.101 | 24.023 | 1.00 | 25.19 | W | O |
| ATOM | 4906 | O | HOH | W | 136 | 4.007 | −9.460 | 34.087 | 1.00 | 38.19 | W | O |
| ATOM | 4909 | O | HOH | W | 137 | −1.239 | −11.368 | 25.339 | 1.00 | 28.01 | W | O |
| ATOM | 4912 | O | HOH | W | 138 | 25.810 | −13.282 | 12.651 | 1.00 | 29.26 | W | O |
| ATOM | 4915 | O | HOH | W | 139 | −11.471 | 3.486 | 23.719 | 1.00 | 43.71 | W | O |
| ATOM | 4918 | O | HOH | W | 140 | −14.572 | 6.189 | 16.547 | 1.00 | 32.73 | W | O |
| ATOM | 4921 | O | HOH | W | 141 | 25.455 | −4.517 | 28.979 | 1.00 | 27.27 | W | O |
| ATOM | 4924 | O | HOH | W | 142 | −2.265 | −18.449 | 18.806 | 1.00 | 31.12 | W | O |
| ATOM | 4927 | O | HOH | W | 143 | 24.881 | −12.339 | 32.109 | 1.00 | 47.48 | W | O |
| ATOM | 4930 | O | HOH | W | 144 | 3.783 | −12.994 | 33.207 | 1.00 | 40.92 | W | O |
| ATOM | 4933 | O | HOH | W | 145 | 16.661 | 4.952 | 10.161 | 1.00 | 41.86 | W | O |
| ATOM | 4936 | O | HOH | W | 146 | −9.286 | −1.522 | 21.390 | 1.00 | 35.03 | W | O |
| ATOM | 4939 | O | HOH | W | 147 | 14.410 | −11.209 | 45.987 | 1.00 | 39.95 | W | O |
| ATOM | 4942 | O | HOH | W | 148 | −1.544 | −13.594 | 23.703 | 1.00 | 33.58 | W | O |
| ATOM | 4945 | O | HOH | W | 149 | 4.387 | 7.952 | 19.690 | 1.00 | 35.86 | W | O |
| ATOM | 4948 | O | HOH | W | 150 | −8.699 | 18.279 | 6.531 | 1.00 | 32.48 | W | O |
| ATOM | 4951 | O | HOH | W | 151 | 3.719 | −3.684 | 38.401 | 1.00 | 30.50 | W | O |
| ATOM | 4954 | O | HOH | W | 152 | 3.775 | −20.312 | 14.291 | 1.00 | 42.65 | W | O |
| ATOM | 4957 | O | HOH | W | 153 | 27.957 | −13.614 | 22.778 | 1.00 | 37.15 | W | O |
| ATOM | 4960 | O | HOH | W | 154 | −6.363 | −9.621 | 22.860 | 1.00 | 31.35 | W | O |
| ATOM | 4963 | O | HOH | W | 155 | 17.426 | −9.113 | 39.788 | 1.00 | 41.82 | W | O |
| ATOM | 4966 | O | HOH | W | 156 | 11.719 | −0.755 | 38.894 | 1.00 | 44.37 | W | O |
| ATOM | 4969 | O | HOH | W | 157 | −1.823 | −18.279 | 23.964 | 1.00 | 31.03 | W | O |
| ATOM | 4972 | O | HOH | W | 158 | 1.236 | 19.857 | 5.620 | 1.00 | 33.82 | W | O |
| ATOM | 4975 | O | HOH | W | 159 | −5.447 | −2.279 | 8.958 | 1.00 | 43.06 | W | O |
| ATOM | 4978 | O | HOH | W | 160 | 6.495 | 1.870 | 6.024 | 1.00 | 32.59 | W | O |
| ATOM | 4981 | O | HOH | W | 161 | −1.902 | −10.079 | 30.428 | 1.00 | 40.56 | W | O |
| ATOM | 4984 | O | HOH | W | 162 | 12.781 | −10.832 | 0.891 | 1.00 | 28.70 | W | O |
| ATOM | 4987 | O | HOH | W | 163 | 15.705 | −18.496 | 12.332 | 1.00 | 42.03 | W | O |
| ATOM | 4990 | O | HOH | W | 164 | 11.477 | 23.027 | 19.258 | 1.00 | 43.18 | W | O |
| ATOM | 4993 | O | HOH | W | 165 | 11.794 | −19.441 | 25.110 | 1.00 | 34.30 | W | O |
| ATOM | 4996 | O | HOH | W | 166 | −9.142 | 6.492 | 24.586 | 1.00 | 37.55 | W | O |
| ATOM | 4999 | O | HOH | W | 167 | −3.791 | −17.095 | 17.004 | 1.00 | 40.54 | W | O |
| ATOM | 5002 | O | HOH | W | 168 | 18.861 | −5.381 | −3.135 | 1.00 | 38.29 | W | O |
| ATOM | 5005 | O | HOH | W | 169 | 1.506 | 21.043 | 36.589 | 1.00 | 44.96 | W | O |
| ATOM | 5008 | O | HOH | W | 170 | 12.593 | −2.711 | −7.043 | 1.00 | 44.98 | W | O |
| ATOM | 5011 | O | HOH | W | 171 | 15.127 | −0.179 | 0.329 | 1.00 | 35.60 | W | O |
| ATOM | 5014 | O | HOH | W | 172 | 15.056 | 2.474 | −0.998 | 1.00 | 37.93 | W | O |
| ATOM | 5017 | O | HOH | W | 173 | −7.283 | −6.499 | 7.178 | 1.00 | 49.30 | W | O |
| ATOM | 5020 | O | HOH | W | 174 | 21.316 | 0.314 | −2.427 | 1.00 | 41.75 | W | O |
| ATOM | 5023 | O | HOH | W | 175 | 26.651 | −5.640 | 2.963 | 1.00 | 36.35 | W | O |
| ATOM | 5026 | O | HOH | W | 176 | 29.087 | −10.247 | 25.287 | 1.00 | 39.54 | W | O |
| ATOM | 5029 | O | HOH | W | 177 | 23.713 | 5.560 | 13.541 | 1.00 | 39.25 | W | O |
| ATOM | 5032 | O | HOH | W | 178 | −1.768 | 14.984 | −1.759 | 1.00 | 40.11 | W | O |
| ATOM | 5035 | O | HOH | W | 179 | 13.927 | −22.767 | 20.107 | 1.00 | 44.51 | W | O |
| ATOM | 5038 | O | HOH | W | 180 | −5.833 | 17.092 | 42.432 | 1.00 | 39.14 | W | O |
| ATOM | 5041 | O | HOH | W | 181 | 4.780 | 28.120 | 30.190 | 1.00 | 35.76 | W | O |
| ATOM | 5044 | O | HOH | W | 182 | 5.232 | −21.397 | 10.130 | 1.00 | 32.74 | W | O |
| ATOM | 5047 | O | HOH | W | 183 | −0.609 | −13.905 | 29.169 | 1.00 | 42.77 | W | O |
| ATOM | 5050 | O | HOH | W | 184 | −8.615 | −12.423 | 20.365 | 1.00 | 38.88 | W | O |
| ATOM | 5053 | O | HOH | W | 185 | −2.742 | 24.869 | 15.943 | 1.00 | 35.44 | W | O |
| ATOM | 5056 | O | HOH | W | 186 | −6.914 | 23.870 | 13.421 | 1.00 | 38.92 | W | O |
| ATOM | 5059 | O | HOH | W | 187 | −9.844 | 5.148 | 9.425 | 1.00 | 47.06 | W | O |
| ATOM | 5062 | O | HOH | W | 188 | −3.377 | 1.908 | 31.500 | 1.00 | 47.62 | W | O |
| ATOM | 5065 | O | HOH | W | 189 | 8.535 | −2.717 | 9.847 | 1.00 | 20.99 | W | O |
| ATOM | 5068 | O | HOH | W | 190 | −2.120 | 23.920 | 11.635 | 1.00 | 33.73 | W | O |
| ATOM | 5071 | O | HOH | W | 191 | −1.821 | 12.406 | 36.174 | 1.00 | 33.31 | W | O |
| ATOM | 5074 | O | HOH | W | 192 | 15.430 | −22.456 | 17.980 | 1.00 | 32.67 | W | O |
| ATOM | 5077 | O | HOH | W | 193 | 26.766 | −7.447 | 10.363 | 1.00 | 37.80 | W | O |
| ATOM | 5080 | O | HOH | W | 194 | 7.871 | −12.672 | 1.551 | 1.00 | 33.24 | W | O |
| ATOM | 5083 | O | HOH | W | 195 | 11.658 | −13.419 | −1.004 | 1.00 | 38.16 | W | O |
| ATOM | 5086 | O | HOH | W | 196 | 16.826 | 1.526 | 0.614 | 1.00 | 50.30 | W | O |
| ATOM | 5089 | O | HOH | W | 197 | 15.595 | 6.152 | 14.916 | 1.00 | 40.35 | W | O |
| ATOM | 5092 | O | HOH | W | 198 | −1.881 | 25.715 | 18.176 | 1.00 | 37.84 | W | O |
| ATOM | 5095 | O | HOH | W | 199 | −11.651 | 11.014 | 33.297 | 1.00 | 32.46 | W | O |
| ATOM | 5098 | O | HOH | W | 200 | 18.893 | −2.239 | 0.202 | 1.00 | 36.54 | W | O |
| ATOM | 5101 | O | HOH | W | 201 | 8.083 | −15.775 | 41.296 | 1.00 | 35.79 | W | O |
| ATOM | 5104 | O | HOH | W | 202 | 27.247 | −8.234 | 2.554 | 1.00 | 45.23 | W | O |
| ATOM | 5107 | O | HOH | W | 203 | −1.222 | −15.328 | 27.055 | 1.00 | 46.27 | W | O |
| ATOM | 5110 | O | HOH | W | 204 | 13.756 | −2.002 | 41.227 | 1.00 | 44.19 | W | O |
| ATOM | 5113 | O | HOH | W | 205 | 18.212 | 11.234 | 28.397 | 1.00 | 46.27 | W | O |
| ATOM | 5116 | O | HOH | W | 206 | 10.446 | 24.528 | 22.495 | 1.00 | 29.18 | W | O |
| ATOM | 5119 | O | HOH | W | 207 | 9.812 | 7.702 | 16.580 | 1.00 | 47.51 | W | O |
| ATOM | 5122 | O | HOH | W | 208 | 8.614 | 13.189 | 26.746 | 1.00 | 42.96 | W | O |
| ATOM | 5125 | O | HOH | W | 209 | 26.242 | 4.872 | 13.201 | 1.00 | 34.36 | W | O |
| ATOM | 5128 | O | HOH | W | 210 | 3.417 | 32.021 | 7.569 | 1.00 | 43.78 | W | O |
| ATOM | 5131 | O | HOH | W | 211 | 13.082 | 12.607 | −0.030 | 1.00 | 38.29 | W | O |
| ATOM | 5134 | O | HOH | W | 212 | 11.113 | −16.047 | 1.534 | 1.00 | 48.53 | W | O |
| ATOM | 5137 | O | HOH | W | 213 | 16.799 | −19.980 | 17.140 | 1.00 | 42.01 | W | O |
| ATOM | 5140 | O | HOH | W | 214 | 8.100 | 2.699 | 9.516 | 1.00 | 38.54 | W | O |
| ATOM | 5143 | O | HOH | W | 215 | 21.192 | −10.596 | 35.721 | 1.00 | 42.09 | W | O |
| ATOM | 5146 | O | HOH | W | 216 | −12.311 | 27.355 | 25.513 | 1.00 | 46.88 | W | O |
| ATOM | 5149 | O | HOH | W | 217 | 2.196 | 18.838 | 8.312 | 1.00 | 40.97 | W | O |
| ATOM | 5152 | O | HOH | W | 218 | 2.760 | 7.802 | 34.907 | 1.00 | 40.65 | W | O |
| ATOM | 5155 | O | HOH | W | 219 | 2.052 | 34.572 | 8.653 | 1.00 | 28.96 | W | O |
| ATOM | 5158 | O | HOH | W | 220 | 20.199 | 10.058 | 26.993 | 1.00 | 39.00 | W | O |
| ATOM | 5161 | O | HOH | W | 221 | 0.666 | 6.917 | 33.693 | 1.00 | 41.05 | W | O |
| ATOM | 5164 | O | HOH | W | 222 | 19.656 | −20.516 | 17.448 | 1.00 | 49.29 | W | O |
| ATOM | 5167 | O | HOH | W | 223 | 24.529 | −13.997 | 28.898 | 1.00 | 44.80 | W | O |
| ATOM | 5170 | O | HOH | W | 224 | −15.502 | 8.924 | 35.463 | 1.00 | 46.22 | W | O |
| ATOM | 5173 | O | HOH | W | 225 | 6.321 | 6.726 | 39.047 | 1.00 | 40.39 | W | O |
| ATOM | 5176 | O | HOH | W | 226 | 13.346 | −19.882 | 30.564 | 1.00 | 27.35 | W | O |
| ATOM | 5179 | O | HOH | W | 227 | 2.475 | 8.509 | 20.962 | 1.00 | 37.71 | W | O |
| ATOM | 5182 | O | HOH | W | 228 | 25.697 | −17.409 | 7.650 | 1.00 | 40.93 | W | O |
| ATOM | 5185 | O | HOH | W | 229 | −5.326 | 22.902 | 36.076 | 1.00 | 34.85 | W | O |
| ATOM | 5188 | O | HOH | W | 230 | 18.689 | 1.894 | 1.969 | 1.00 | 40.05 | W | O |
| ATOM | 5191 | O | HOH | W | 231 | 22.256 | 9.449 | 29.382 | 1.00 | 38.70 | W | O |
| ATOM | 5194 | O | HOH | W | 232 | 6.671 | 16.029 | 32.045 | 1.00 | 45.79 | W | O |
| ATOM | 5197 | O | HOH | W | 233 | −12.901 | 11.354 | 24.852 | 1.00 | 42.77 | W | O |
| ATOM | 5200 | O | HOH | W | 234 | 11.146 | −21.564 | 4.017 | 1.00 | 44.94 | W | O |
| ATOM | 5203 | O | HOH | W | 235 | 25.034 | −1.313 | 25.290 | 1.00 | 35.63 | W | O |
| ATOM | 5206 | O | HOH | W | 236 | −14.460 | 18.497 | 19.730 | 1.00 | 43.85 | W | O |
| ATOM | 5209 | O | HOH | W | 237 | −12.580 | 11.671 | 30.829 | 1.00 | 49.50 | W | O |
| ATOM | 5212 | O | HOH | W | 238 | −15.352 | 9.953 | 23.232 | 1.00 | 41.57 | W | O |
| ATOM | 5215 | O | HOH | W | 239 | −1.668 | 11.121 | 33.395 | 1.00 | 56.23 | W | O |
| ATOM | 5218 | O | HOH | W | 240 | 8.754 | −13.126 | −3.263 | 1.00 | 52.79 | W | O |
| ATOM | 5221 | O | HOH | W | 241 | −1.876 | −3.651 | 6.368 | 1.00 | 34.87 | W | O |
| ATOM | 5224 | O | HOH | W | 242 | 19.512 | −18.187 | 6.893 | 1.00 | 42.20 | W | O |
| ATOM | 5227 | O | HOH | W | 243 | −8.077 | 30.761 | 18.011 | 1.00 | 33.22 | W | O |
| ATOM | 5230 | O | HOH | W | 244 | 11.861 | 14.174 | 19.992 | 1.00 | 49.09 | W | O |
| ATOM | 5233 | O | HOH | W | 245 | 0.301 | 11.850 | −6.763 | 1.00 | 46.36 | W | O |
| ATOM | 5236 | O | HOH | W | 246 | −2.640 | 12.555 | 0.451 | 1.00 | 48.51 | W | O |
| ATOM | 5239 | O | HOH | W | 247 | 12.588 | −19.822 | 12.376 | 1.00 | 48.93 | W | O |
| ATOM | 5242 | O | HOH | W | 248 | 11.738 | 20.944 | 27.362 | 1.00 | 51.67 | W | O |
| ATOM | 5245 | O | HOH | W | 249 | 17.071 | 8.568 | 19.617 | 1.00 | 43.62 | W | O |
| ATOM | 5248 | O | HOH | W | 250 | −2.350 | −17.778 | 11.732 | 1.00 | 51.64 | W | O |
| ATOM | 5251 | O | HOH | W | 251 | 12.306 | −4.700 | 40.223 | 1.00 | 48.28 | W | O |
| END | | | | | | | | | | | | |

[image] TABLE 6

PYK2 in pET15S

U33284; Human protein tyrosine kinase PYK2 mRNA, complete cds
Full-length protein in pET15S: 293 aa (SEQ ID NO:2) Mass: 33872.2 pI: 6.07
PYK2 kinase domain I420-M691 (not including first 21 aa in following sequence) SEQ ID
NO:1

| | | | | | | |
|---|---|---|---|---|---|---|
| 1 | MGSSHHHHHH | SSGLVPRGSH | MIAREDVVLN | RILGEGFFGE | VYEGVYTNHK | GEKINVAVKT |
| 61 | CKKDCTLDNK | EKFMSEAVIM | KNLDHPHIVK | LIGIIEEEPT | WIIMELYPYG | ELGHYLERNK |
| 121 | NSLKVLTLVL | YSLQICKAMA | YLESINCVHR | DIAVRNILVA | SPECVKLGDF | GLSRYIEDED |
| 181 | YYKASVTRLP | IKWMSPESIN | FRRFTTASDV | WMFAVCMWEI | LSFGKQPFFW | LENKDVIGVL |
| 241 | EKGDRLPKPD | LCPPVLYTLM | TRCWDYDPSD | RPRFTELVCS | LSDVYQMEKD | IAM |

| | | |
|---|---|---|
| SEQ ID NO:5 | | |
| PYK2-C1; | 5′-TCCACAG CATATG ATTGCCCGTGAAGATGTGGT-3′ | 33 mer |
| SEQ ID NO:6 | | |
| PYK2-N2; | TGGAGAAGGACATTGCCATG TAG GTCGAC GAGAG (Origin) | |
| | 5′-CTCTC GTCGAC CTA CATGGCAATGTCCTTCTCCA-3′ | 34 mer |

pET15S sequence PCR product; 843 bp (SEQ ID NO: 4)
Sequence encoding PYK2 kinase domain (in small letters below) (SEQ ID NO: 3)
TCTAGAAATAATTTTGTTTAACTTTAAGAAGGAGA
TATACCATGGGCAGCAGCCATCATCATCATCATCACAGCAGCGGCCTGGTGCCGCGCGGCAGCCATATG

| | | | | | | |
|---|---|---|---|---|---|---|
| | attgcc | cgtgaagatg | | | | |
| 1381 | tggtcctgaa | tcgtattctt | ggggaaggct | tttttgggga | ggtctatgaa | ggtgtctaca |
| 1441 | caaatcacaa | aggggagaaa | atcaatgtag | ctgtcaagac | ctgcaagaaa | gactgcactc |
| 1501 | tggacaacaa | ggagaagttc | atgagcgagg | cagtgatcat | gaagaacctc | gaccacccgc |
| 1561 | acatcgtgaa | gctgatcggc | atcattgaag | aggagcccac | ctggatcatc | atggaattgt |
| 1621 | atccctatgg | ggagctgggc | cactacctgg | agcggaacaa | gaactccctg | aaggtgctca |
| 1681 | ccctcgtgct | gtactcactg | cagatatgca | aagccatggc | ctacctggag | agcatcaact |
| 1741 | gcgtgcacag | ggacattgct | gtccggaaca | tcctggtggc | ctcccctgag | tgtgtgaagc |
| 1801 | tgggggactt | tggtctttcc | cggtacattg | aggacgagga | ctattacaaa | gcctctgtga |
| 1861 | ctcgtctccc | catcaaatgg | atgtccccag | agtccattaa | cttccgacgc | ttcacgacag |
| 1921 | ccagtgacgt | ctggatgttc | gccgtgtgca | tgtgggagat | cctgagcttt | gggaagcagc |
| 1981 | ccttcttctg | gctggagaac | aaggatgtca | tcggggtgct | ggagaaagga | gaccggctgc |
| 2041 | ccaagcctga | tctctgtcca | ccggtccttt | ataccctcat | gacccgctgc | tgggactacg |
| 2101 | accccagtga | ccggccccgc | ttcaccgagc | tggtgtgcag | cctcagtgac | gtttatcaga |
| 2161 | tggagaagga | cattgccatg | | | | |

TAGGTCGACTAGAGCCTGCAGTCTCGACCATCATCATCATCATCATTAATAAAAGGGCG
AATTCCAGCACACTGGCGGCCGTTACTAGTGGATCCGGCTGCTAACAAAGCCCGAAAGGAAGCTGAGTTGG

TABLE 7
Pyk2 Activity and the Inhibition by ATP Analogs

| Pyk2 | Vmax | Vmax (SE) | K | K (SE) | K (Lo 95%) | K (Up 95%) | Equation |
|---|---|---|---|---|---|---|---|
| 8 ng/well | 1.25e+4 | 9.11e+2 | 7.37e+0 | 2.79e+0 | 3.27e+0 | 1.66e+1 | y = (Vmax * x)/(K + x) |

| Compounds | Vmax | K | K (SE) | K (Lo 95%) | K (Up 95%) | Y2 | n | Equation |
|---|---|---|---|---|---|---|---|---|
| Adenosine | 1.82e+4 | 2.54e+2 | 2.65e+2 | 2.47e+1 | 2.60e+3 | 7.33e+2 | −5.14e−1 | y = ((Vmax * x\hat{ }n)/(K\hat{ }n + x\hat{ }n)) + Y2 |
| AMP | 1.82e+4 | 8.02e+1 | 3.76e+1 | 2.82e+1 | 2.28e+2 | 7.33e+2 | −5.05e−1 | y = ((Vmax * x\hat{ }n)/(K\hat{ }n + x\hat{ }n)) + Y2 |
| ADT | 1.82e+4 | 1.49e+1 | 2.69e+0 | 9.93e+0 | 2.22e+1 | 7.33e+2 | −7.69e−1 | y = ((Vmax * x\hat{ }n)/(K\hat{ }n + x\hat{ }n)) + Y2 |
| AMPPCP | 1.82e+4 | 7.69e+3 | 1.99e+4 | 2.43e+1 | 2.44e+6 | 7.33e+2 | −2.03e−1 | y = ((Vmax * x\hat{ }n)/(K\hat{ }n + x\hat{ }n)) + Y2 |
| AMPPNP | 1.82e+4 | 1.81e+1 | 2.82e+0 | 1.28e+1 | 2.56e+1 | 7.33e+2 | −7.18e−1 | y = ((Vmax * x\hat{ }n)/(K\hat{ }n + x\hat{ }n)) + Y2 |
| ATP-g-S | 1.82e+4 | 1.36e+1 | 1.49e+0 | 1.06e+1 | 1.73e+1 | 7.33e+2 | −9.66e−1 | y = ((Vmax * x\hat{ }n)/(K\hat{ }n + x\hat{ }n)) + Y2 |

What is claimed is:

1. A method of designing a ligand that binds to at least one target molecule that is a member of a protein family, comprising
identifying as molecular scaffolds one or more compounds that bind to a binding site of the target molecule with low affinity;
determining the orientation of the one or more molecular scaffolds at the binding site of the target molecule by obtaining co-crystal structures of the molecular scaffolds in the binding site;
identifying one or more structures of at least one scaffold molecule that, when modified, provide a ligand having altered binding affinity or binding specificity or both for binding to the target molecule as compared to the binding of the scaffold molecule;
providing at least one said ligand.

2. The method of claim 1, wherein said one or more molecular scaffolds bind with low affinity to a plurality of target molecules in said protein family.

3. The method of claim 1, wherein said one more molecular scaffolds bind with very low affinity to a plurality of target molecules in said protein family.

4. The method of claim 1, wherein providing the ligand comprises chemical synthesis.

5. The method of claim 1, further comprising that a plurality of distinct compounds are assayed for binding to the binding site of the target molecules.

6. The method of claim 1, further comprising that co-crystals of the molecular scaffolds bound to the target molecule are isolated, and the orientation of the molecular scaffold is determined by performing X-ray crystallography on the co-crystals.

7. The method of claim 1, further comprising identifying common chemical structures of the molecular scaffolds and placing the molecular scaffolds into groups based on having at least one common chemical structure; and
determining the orientation of the one or more molecular scaffolds at the binding site of the target molecule for at least one representative compound from a plurality of groups.

8. The method of claim 1, wherein the ligand binds to the target molecule with greater binding affinity or greater binding specificity or both than the molecular scaffold.

9. The method of claim 1, wherein the orientation of the molecular scaffold is determined by nuclear magnetic resonance in co-crystal structure determination.

10. The method of claim 5, wherein the plurality of distinct compounds are each assayed for binding to a plurality of members of the molecular family.

11. The method of claim 5, wherein the distinct compounds have a molecular weight of from about 100 to about 350 daltons.

12. The method of claim 1, wherein the target molecule is a protein and the molecular family is a protein family.

13. The method of claim 12, wherein the protein is an enzyme.

14. The method of claim 5, wherein the distinct compounds have a molecular weight of from about 150 to about 350 daltons.

15. The method of claim 5, wherein the distinct compounds comprise a ring structure.

16. The method of claim 12, wherein the protein family is selected from the group consisting of: protein kinases, proteases, and phosphatases.

17. The method of claim 7, further comprising that after the identification of common chemical structures of the distinct compounds that bind, the compounds are grouped into classes based on common chemical structures and a representative compound from a plurality of the classes is selected for performing X-ray crystallography on co-crystals of the compound and target molecule.

18. The method of claim 14, wherein the distinct compounds are selected based on criteria selected from the group consisting of: molecular weight, clogP, and the number of hydrogen bond donors and acceptors.

19. The method of claim 18, wherein the clog P is less than 2, and the number of hydrogen bond donors and acceptors is less than 5.

20. The method of claim 5, wherein the assay is an enzymatic assay.

21. The method of claim 17, wherein the number of classes is at least 200.

22. The method of claim 1, wherein the binding of the ligand to the target molecule causes a specific biochemical effect due to the inhibition of an enzyme.

23. The method of claim 1, wherein the modification is the addition, subtraction, or substitution of a chemical group.

24. The method of claim 1, wherein the modification causes the ligand to be actively transported to particular cells and/or a particular organ.

25. The method of claim 1, wherein the modification of the compound comprises the addition or subtraction of a chemical group selected from the group consisting of: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (forming a ketone or N-oxide) and a sulphur atom (forming a thione).

26. The method of claim 6, further comprising that information provided by performing X-ray crystallography on the co-crystals is provided to a computer program, wherein the computer program provides a prediction of changes in the interaction between the molecular scaffold and the protein that result from specific modifications to the molecular scaffold, and the molecular scaffold is chemically modified based on the prediction of the biochemical result.

27. The method of claim 26, wherein the computer program provides the prediction based on a virtual assay selected from the group consisting of: virtual docking of the compound to the protein, shape-based matching, free energy perturbations, and three-dimensional pharmacophore.

28. The method of claim 1, wherein when a chemically tractable structure of the compound is modified, a ligand is provided that fills a void volume in the protein-ligand complex.

29. The method of claim 1, wherein when a chemically tractable structure of the compound is modified, an attractive ionic charge is produced in the protein-ligand complex.

30. The method of claim 1, further providing that the modification results in a sub-structure of the ligand being present in a binding pocket of the protein binding site when the protein-ligand complex is formed.

31. The method of claim 6, further providing that after identifying the common chemical structures of the compounds that bind, the compounds are grouped based on comprising a common chemical sub-structure and a representative compound from a plurality of groups is selected for co-crystallization with the protein and performance of the X-ray crystallography.

32. The method of claim 6, wherein the X-ray crystallography and the modification of a chemically tractable structure of the compound are each performed a plurality of times.

33. The method of claim 1, wherein said molecular scaffold binds to said target molecule with very low affinity.

34. The method of claim 1, wherein said molecular scaffold binds to said target molecule with extremely low affinity.

35. The method of claim 1, further comprising identifying conserved residues in said binding sites that interact with said molecular scaffold.

36. The method of claim 35, wherein identifying conserved residues comprises identifying binding site residues that are identical for a plurality of members of said molecular family in sequence alignments of said plurality of members.

37. The method of claim 36, wherein identifying conserved residues that interact with said molecular scaffold comprises identifying conserved residues within 5 angstroms of said molecular scaffold in a co-crystal of said molecular scaffold and target molecule.

38. A method of designing a ligand that binds to at least one target molecule that is a member of a molecular family, comprising, identifying as molecular scaffolds one or more compounds that bind to binding sites of a plurality of members of a molecular family;
determining the orientation of the one or more molecular scaffolds at the binding site of the target molecule to identify chemically tractable structures of the scaffolds that, when modified, alter the binding affinity or binding specificity between the scaffold and the target molecule;
synthesizing a ligand wherein one or more of the chemically tractable structures of the molecular scaffold is modified to provide a ligand that binds to the target molecule with altered binding affinity or binding specificity.

39. The method of claim 38, wherein said molecular scaffold binds to said target molecule with low affinity.

40. The method of claim 38, wherein said molecular scaffold binds to said target molecule with very low affinity.

41. The method of claim 38, wherein said molecular scaffold binds to said target molecule with extremely low affinity.

42. The method of claim 38, wherein said molecular scaffold binds to said target molecule with moderate affinity.

43. The method of claim 38, further comprising that co-crystals of the molecular scaffolds bound to the target molecule are isolated, and the orientation of a molecular scaffolds at the binding site of the protein is determined by performing X-ray crystallography on the co-crystals.

44. The method of claim 38, further comprising that common chemical structures of the molecular scaffolds are identified and that the molecular scaffolds are placed into groups based on having at least one common chemical structure; and
the step of determining the orientation of the one or more molecular scaffolds at the binding site of the target molecule is determined for at least one representative compound from each group.

45. The method of claim 38, wherein the ligand binds to the target molecule with greater binding affinity or binding specificity than the molecular scaffold.

46. The method of claim 38, wherein the orientation of the molecular scaffold is determined by nuclear magnetic resonance.

47. The method of claim 38, wherein a plurality of distinct compounds are each assayed for binding to a plurality of members of the molecular family.

48. The method of claim 47, wherein the distinct compounds have a molecular weight of from about 100 to about 350 daltons.

49. The method of claim 38, wherein the target molecule is a protein and the molecular family is a protein family.

50. The method of claim 49, wherein the protein is an enzyme.

51. The method of claim 48, wherein the distinct compounds have a molecular weight of from about 150 to about 350 daltons.

52. The method of claim 48, wherein the distinct compounds comprise a ring structure.

53. The method of claim 49, wherein the protein family is selected from the group consisting of: protein kinases, proteases, and phosphatases.

54. The method of claim 47, wherein at least about 5% of the compounds bind with low affinity.

55. The method of claim 47, wherein at least about 10% of the compounds bind with low affinity.

56. The method of claim 44, further comprising that after the identification of common chemical structures of the distinct compounds that bind, the compounds are grouped into classes based on common chemical structures and a representative compound from a plurality of the classes is selected for performing the X-ray crystallography.

57. The method of claim 47, wherein the distinct compounds are selected based on criteria selected from the group consisting of: molecular weight, clogP, and the number of hydrogen bond donors and acceptors.

58. The method of claim 57, wherein the molecular weight is from about 150 to about 350 daltons, the clog P is less than 2, and the number of hydrogen bond donors and acceptors is less than 5.

59. The method of claim 38, wherein the assay is an enzymatic assay.

60. The method of claim 56, wherein the number of groups is at least 200.

61. The method of claim 38, wherein the binding of the ligand to the target molecule causes a specific biochemical effect due to the inhibition of an enzyme.

62. The method of claim 38, wherein the modification is the addition, subtraction, or substitution of a chemical group.

63. The method of claim 38, wherein the modification causes the scaffold to be actively transported to particular cells and/or a particular organ.

64. The method of claim 38, wherein the modification of the compound comprises the addition or subtraction of a chemical group selected from the group consisting of: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (forming a ketone or N-oxide) and a sulphur atom (forming a thione).

65. The method of claim 43, further comprising that information provided by performing X-ray crystallography on the co-crystals is provided to a computer program, wherein the computer program provides a prediction of changes in the interaction between the molecular scaffold and the protein that result from specific modifications to the molecular scaffold, and the molecular scaffold is chemically modified based on the prediction of the biochemical result.

66. The method of claim 65, wherein the computer program provides the prediction based on a virtual assay selected from the group consisting of: virtual docking of the compound to the protein, shape-based matching, free energy perturbations, and three-dimensional pharmacophore.

67. The method of claim 38, wherein when a chemically tractable structure of the compound is modified, a ligand is provided that fills a void volume in the protein-ligand complex.

68. The method of claim 38, wherein when a chemically tractable structure of the compound is modified, an attractive ionic charge is produced in the protein-ligand complex.

69. The method of claim 38, further providing that the modification results in a sub-structure of the ligand being present in a binding pocket of the protein binding site when the protein-ligand complex is formed.

70. The method of claim 44, further providing that after identifying the common chemical structures of the compounds that bind, the compounds are grouped based on comprising a common chemical sub-structure and a representative compound from each group is selected for co-crystallization with the protein and performance of the X-ray crystallography.

71. The method of claim 44, wherein the X-ray crystallography and the modification of a chemically tractable structure of the compound are each performed a plurality of times.

72. The method of claim 38, further comprising identifying conserved residues in said binding sites that interact with said molecular scaffold.

73. The method of claim 38, wherein identifying conserved residues comprises identifying binding site residues that are identical for a plurality of members of said molecular family in sequence alignments of said plurality of members.

74. The method of claim 72, wherein identifying conserved residues that interact with said molecular scaffold comprises identifying conserved residues within 5 angstroms of said molecular scaffold in a co-crystal of said molecular scaffold and target molecule.

75. A method for identifying binding characteristics of a ligand of a target protein, comprising
identifying at least one conserved interacting residue in said target protein that interacts with at least two binding molecules; and
identifying at least one common interaction property of said at least two binding molecules with said conserved residue, thereby identifying at least one said characteristic.

76. The method of claim 75, wherein identifying at least one conserved interacting residue comprises comparing a plurality of amino acid sequences in a protein family to which said target protein belongs and identifying binding site residues conserved in said protein family.

77. The method of claim 75, wherein identifying at least one conserved interacting residue comprises identifying conserved residues within 5 angstroms of said at least two binding molecules in co-crystals of said binding molecules and target protein.

78. The method of claim 75, wherein said interaction property is selected from the group consisting of hydrophobic interaction, charge-charge interaction, hydrogen bonding, charge-polar interaction, polar-polar interaction, and combinations thereof.

79. A method for identifying energetically allowed sites on a binding compound for attachment of an additional component, comprising analyzing the orientation of said binding compound in a target binding site, thereby identifying accessible sites on said compound of said separate component.

80. The method of claim 79, further comprising calculating the free energy cost of attachment of said separate component at one or more of said accessible sites.

81. The method of claim 79, wherein said orientation is determined by co-crystallography.

82. The method of claim 79, wherein said separate component comprises a linker.

83. The method of claim 79, wherein said separate component comprises a label.

84. The method of claim 79, wherein said separate component comprises a solid phase material.

85. A method for attaching a binding compound to an attachment component, comprising
identifying energetically allowed sites for attachment of a said attachment component on a binding compound; and
attaching said compound or derivative thereof to said attachment component at said energetically allowed site.

86. The method of claim 85, wherein said attachment component is a linker for attachment to a solid phase medium, and said method further comprises attaching said compound or derivative to a solid phase medium through said linker attached at a said energetically allowed site.

87. The method of claim 86, wherein said linker is a traceless linker.

88. The method of claim 86, wherein said binding compound or derivative thereof is synthesized on a said linker attached to said solid phase medium.

89. The method of claim 88, wherein a plurality of said compounds or derivatives are synthesized in combinatorial synthesis.

90. The method of claim 86, wherein attachment of said compound to said solid phase medium provides an affinity medium.

91. The method of claim 85, wherein said attachment component comprises a label.

92. The method of claim 91, wherein said label comprises a fluorophore.

93. A method for making an affinity matrix, comprising
identifying energetically allowed sites on a target binding compound for attachment to a solid phase matrix; and
attaching said target binding compound to said solid phase matrix through a said energetically allowed site.

94. The method of claim 93, further comprising determining the orientation of said target binding compound in a binding site in said target.

95. The method of claim 93, wherein identifying energetically allowed sites comprises calculating the change in free energy for attachment of said target binding compound to said solid phase matrix.

96. The method of claim 93, wherein said target binding compound is attached to said solid phase matrix through a linker.

97. The method of claim 93, wherein said solid phase matrix is selected from the group consisting of gel, bead, plate, chip, and well.

98. The method of claim 93, wherein identifying energetically allowed sites for attachment to a solid phase matrix is performed for at least 10 different compounds.


SEQUENCE LISTING (38 sequences)

SEQ ID NO: 1 - protein, 272 aa, Homo sapiens
IAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAM

SEQ ID NO: 2 - protein, 293 aa, Artificial Sequence
  Description of Artificial Sequence Synthetic pET15S protein containing PYK2
MGSSHHHHHHSSGLVPRGSHMIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAM

SEQ ID NO: 3 - DNA, 816 bp, Homo sapiens
attgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatg

SEQ ID NO: 4 - DNA, 1050 bp, Artificial Sequence
  Description of Artificial Sequence Synthetic pET15S nucleotide sequence
tctagaaataattttgtttaactttaagaaggagatataccatgggcagcagccatcatcatcatcatcacagcagcggcctggtgccgcgcggcagccatatgattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatgtaggtcgactagagcctgcagtctcgaccatcatcatcatcatcattaataaaagggcgaattccagcacactggcggccgttactagtggatccggctgctaacaaagcccgaaaggaagctgagttgg

SEQ ID NO: 5 - DNA, 33 bp, Artificial Sequence
  Description of Artificial Sequence Primer
tccacagcatatgattgcccgtgaagatgtggt

SEQ ID NO: 6 - DNA, 34 bp, Artificial Sequence
  Description of Artificial Sequence Primer
ctctcgtcgacctacatggcaatgtccttctcca

SEQ ID NO: 7 - DNA, 391 bp, Artificial Sequence
  CDS (108)..(170)
  Description of Artificial Sequence Synthetic pET15S multi-cloning site nucleotide sequence
agatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgggatccggaattcaaaggcctacgtcgactagagcctgcagtctcgaccatcatcatcatcatcattaataaaagggcgaattccagcacactggcggccgttactagtggatccggctgctaacaaagcccgaaaggaagctgagttggctgctgccaccgctgagcaataactagcataaccccttggggcctctaaacgggtcttgaggggttttttg

SEQ ID NO: 8 - protein, 21 aa, Artificial Sequence
  Description of Artificial Sequence Synthetic pET15S multi-cloning site peptide sequence
MGSSHHHHHHSSGLVPRGSHM

SEQ ID NO: 9 - protein, 313 aa, Homo sapiens
MLLSKINSLAHLRAAPCNDLHATKLAPGKEKEPLESQYQVGPLLGSGGFGSVYSGIRVSDNLPVAIKHVEKDRISDWGELPNGTRVPMEVVLLKKVSSGFSGVIRLLDWFERPDSFVLILERPEPVQDLFDFITERGALQEELARSFFWQVLEAVRHCHNCGVLHRDIKDENILIDLNRGELKLIDFGSGALLKDTVYTDFDGTRVYSPPEWIRYHRYHGRSAAVWSLGILLYDMVCGDIPFEHDEEIIRGQVFFRQRVSSECQHLIRWCLALRPSDRPTFEEIQNHPWMQDVLLPQETAEIHLHSLSPGPSK

SEQ ID NO: 10 - protein, 313 aa, Mus musculus
MLLSKINSLAHLRARPCNDLHATKLAPGKEKEPLESQYQVGPLLGSGGFGSVYSGIRVADNLPVAIKHVEKDRISDWGELPNGTRVPMEVVLLKKVSSDFSGVIRLLDWFERPDSFVLILERPEPVQDLFDFITERGALQEDLARGFFWQVLEAVRHCHNCGVLHRDIKDENILIDLSRGEIKLIDFGSGALLKDTVYTDFDGTRVYSPPEWIRYHRYHGRSAAVWSLGILLYDMVCGDIPFEHDEEIIKGQVFFRQTVSSECQHLIKWCLSLRPSDRPSFEEIRNHPWMQGDLLPQAASEIHLHSLSPGSSK

SEQ ID NO: 11 - protein, 323 aa, Homo sapiens
MLTKPLQGPPAPPGTPTPPPGGKDREAFEAEYRLGPLLGKGGFGTVFAGHRLTDRLQVAIKVIPRNRVLGWSPLSDSVTCPLEVALLWKVGAGGGHPGVIRLLDWFETQEGFMLVLERPLPAQDLFDYITEKGPLGEGPSRCFFGQVVAAIQHCHSRGVVHRDIKDENILIDLRRGCAKLIDFGSGALLHDEPYTDFDGTRVYSPPEWISRHQYHALPATVWSLGILLYDMVCGDIPFERDQEILEAELHFPAHVSPDCCALIRRCLAPKPSSRPSLEEILLDPWMQTPAEDVTPQPLQRRPCPFGLVLATLSLAWPGLAPNG

SEQ ID NO: 12 - protein, 311 aa, Mus musculus
MLTKPLQGHPSPPVTPTQPPGGKDRAAFEAEYRLGPLLGKGGFGTVFAGHRVTDRRQVAIKVISRNRVLGWSTVSDSVTCPLEVALLWKVGEGNGHPGVIRLLDWFETPEGFMLVLERPMPAQDLFDYITEKGPLGESCSRSFFTQVVAAVQHCHARGVVHRDIKDENILIDLCRGSIKLIDFGSGALLHDEPYTDFDGTRVYSPPEWISRHQYHALPATVWSLGVLLYDMVCGDIPFERDQEILEAELHFPAHVSPDCCALIRRCLAPKPCSRPSLEEILLDPWMQSPAEEKPINSSKGSPTPLPWSLLP

SEQ ID NO: 13 - protein, 297 aa, Homo sapiens
MADKESFEKAYQVGAVLGSGGFGTVYAGSRIADGLPVAVKHVVKERVTEWGSLGGATVPLEVVLLRKVGAAGGARGVIRLLDWFERPDGFLLVLERPEPAQDLFDFITERGALDEPLARRFFAQVLAAVRHCHSCGVVHRDIKDENLLVDLRSGELKLIDFGSGALLKDTVYTDFDGTRVYSPPEWIRYHRYHGRSATVWSLGVLLYDMVCGDIPFEQDEEILRGRLLFRRRVSPECQQLIRWCLSLRPSERPSLDQIAAHPWMLGADGGAPESCDLRLCTLEPDDVASTTSSSESL

SEQ ID NO: 14 - protein, 326 aa, Mus musculus
MLLSKFGSLAHLCGPGGVDHLPVKILQPAKADKESFEKVYQVGAVLGSGGFGTVYAGSRIADGLPVAVKHVVKERVTEWGSLGGVAVPLEVVLLRKVGAAGGARGVIRLLDWFERPDGFLLVLERPEPAQDLFDFITERGALDEPLARRFFAQVLAAVRHCHNCGVVHRDIKDENLLVDLRSGELKLIDFGSGAVLKDTVYTDFDGTRVYSPPEWIRYHRYHGRSATVWSLGVLLYDMVCGDIPFEQDEEILRGRLFFRRRVSPECQQLIEWCLSLRPSERPSLDQIAAHPWMLGTEGSVPENCDLRLCALDTDDGASTTSSSESL

SEQ ID NO: 15 - protein, 323 aa, Colurnix colurnix
MLLSKFGSLAHICSPASMDHLPVKILPPVKVEKEPFDKVYQVGSVLGSGGFGTVYAGSRTADGLPVAVKHVVKERVTEWGTIGGVMVPLEIVLLKKVGSGFRGVIKLLDWYERPDGFLIVMERPELVKDLFDFITEKGALDEDTARGFFRQVLEAVRHCYGCGVVHRDIKDENLLVDLRTGELKLIDFGSGALLKDTVYTDFDGTRVYSPPEWIRYHRYHGRSATVWSLGVLLYDMVCGDIPFEQDEEILRGRLYFRRRISPECQQLIKWCLSLRPSDRPTLEQIFDHQWMHKSEVVKSEDCDIRLRTLDTDVSSTSSSNESL

SEQ ID NO: 16 - protein, 323 aa, Xenopus laevis
MLLSKFGSLAHICNPSNMEHLPVKILQPVKVDKEPFEKVYQVGSVVASGGFGTVYSDSRIADGQPVAVKHVAKERVTEWGTLNGVMVPLEIVLLKKVPTAFRGVINLLDWYERPDAFLIVMERPEPVKDLFDYITEKGPLDEDTARGFFRQVLEAVRHCYNCGVVHRDIKDENLLVDTRNGELKLIDFGSGALLKDTVYTDFDGTRVYSPPEWVRYHRYHGRSATVWSLGVLLYDMVYGDIPFEQDEEIVRVRLCFRRRISTECQQLIKWCLSLRPSDRPTLEQIFDHPWMCKCDLVKSEDCDLRLRTIDNDSSSTSSSNESL

SEQ ID NO: 17 - protein, 310 aa, Danlo rerio
MLDKRIVDVRLDQLEILKAKNGKEHFEKQYTMGNLLGSGGFGSVYSGHRISDGQKVAIKQISRDRIQQWSKMPGEVNPVPNEIALLQSLGGGSGSVPGHRGIIRMLDWFEIPGQEYLIVFEKPQHCQDLFDFITERGRLDESLARRFLKQVIEAVQFCHSKGIVHRDIKDENILVDTRTGDIKVIDFGSGATLKDSMYTDFEGTRVYSPPEWILYHKYHALPLTVWSLGVLLYDMVCGDIPFEQDTDIVKAKPSFNKRISNDCRSLICSCLSYNPGDRPSLEQILQHPWMMESSVDNGDLQEESKIKPSL

SEQ ID NO: 18 - protein, 322 aa, Caenorhabditis elegans
MKKLASLQFFNLKLLLNGESSRGFSKFKKNYKLKAELGRGGFGVVYRAVRTCDNALVAVKFIERSNVKEWARINGEQVPMEICMLAKCSKVRGVIRLLDWYSIPEGFLIVMERPYPCIDMFDFIKGQGKISEDMARFLFRQIAVTVHECVQNRVLHRDLKDENIVIDLVTGSTKLIDFGAATVLRRSQYSDFQGTRLYCPPEWFLHSLYLGREAAVWSLGVLLYNSLNGRLPFRNEKDICTAHLLGPLPFFVPVSAEVKDLISKCLTFDPFQRCSLEAILNHPWVKQQTLSWDALTKNKVQKKTSESSDDHHSETLGDHSET

SEQ ID NO: 19 - protein, 323 aa, Caenorhabditis elegans
MIKRKLQDLAVCCSYQVDFLHEKKHSVKEFKRKYEVLDEIGRGGFGIVYEATTRQDGQQPVAVKFVQHKHVRSWTMTCRQLIPSEVCHLETCEDIPGVIKILDWFANSKGFLIVMERPANCMDLFDMVSVHGPLNEDMGKFIFKQVITTVFNMYSKHGLLHRDIKDENLIVNMNTGEVKLVDFGATAYAEKATKKEFQGTRSYCPPEWFRDQLYLPLEATSWSLGVLLFILLTGKLPFRNEIQICLGNVKFPPDLSKEVCQLVKSCLTTSTSARASLAQIAAHPWMETDKPFFGGDLTFEEALMEIKNPPVVIEDKEAEEEDN

SEQ ID NO: 20 - protein, 273 aa, Homo sapiens
MIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAM

SEQ ID NO: 21 - protein, 310 aa, Unknown Organism
  Description of Unknown Organism FAK tyrosine kinase sequence
EIQRERIELGRCIGEGQFGDVHQGIYMSPENPALAVAIKTCKNCTSDSVREKFLQEACHYTSLHWNWCRYISDPNVDACPDPRNAELTMRQFDHPHIVKLIGVITENPVWIIMELCTLGELRSFLQVRKYSLDLASLILYAYQLSTALAYLESKRFVHRDIAARNVLVSSNDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKAQQEERMRMESR

SEQ ID NO: 22 - protein, 273 aa, Unknown Organism
  Description of Unknown Organism SRC tyrosine kinase sequence
EIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 23 - protein, 271 aa, Unknown Organism
  Description of Unknown Organism HCK tyrosine kinase sequence
EIPRESLKLEKKLGAGQFGEVWMATYNKHTKVAVKTMKPGSMSVEAFLAEANVMKTLQHDKLVKLHAVVTKEPIYIITEFMAKGSLLDFLKSDEGSKQPLPKLIDFSAQIAEGMAFIEQRNYIHRDLRAANILVSASLVCKIADFGLARVIEDNEYTAREGAKFPIKWTAPEAINFGSFTIKSDVWSFGILLMEIVTYGRIPYPGMSNPEVIRALERGYRMPRPENCPEELYNIMMRCWKNRPEERPTFEYIQSVLDDFYTATESQYEEIP

SEQ ID NO: 24 - protein, 263 aa, Unknown Organism
  Description of Unknown Organism LCK tyrosine kinase sequence
EVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTAT

SEQ ID NO: 25 - protein, 263 aa, Unknown Organism
  Description of Unknown Organism ABL1 tyrosine kinase sequence
EMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQ

SEQ ID NO: 26 - protein, 260 aa, Unknown Organism
  Description of Unknown Organism CSK tyrosine kinase sequence
ALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHEL

SEQ ID NO: 27 - protein, 295 aa, Unknown Organism
  Description of Unknown Organism TEK tyrosine kinase sequence
VLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFT

SEQ ID NO: 28 - protein, 291 aa, Unknown Organism
  Description of Unknown Organism KDR tyrosine kinase sequence
EFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKVAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANA

SEQ ID NO: 29 - protein, 290 aa, Unknown Organism
  Description of Unknown Organism FGFR1 tyrosine kinase sequence
ELPRDRLVLGKPLGEGAFGQVVLAEAIGLDKDKPNRVTKVAVKMLKSDATEKDLSDLISEMEMMKMIGKHKNIINLLGACTQDGPLYVIVEYASKGNLREYLQARRPPGLEYSYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALT

SEQ ID NO: 30 - protein, 294 aa, Unknown Organism
  Description of Unknown Organism INSR tyrosine kinase sequence
EVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 31 - protein, 294 aa, Homo sapiens
EVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLLELMRMCWQYNPKMRPSFLEIISSIKEEMEPGFREVSFYYSEENK

SEQ ID NO: 32 - protein, 278 aa, Unknown Organism
  Description of Unknown Organism EPHB2 tyrosine kinase sequence
EIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKA

SEQ ID NO: 33 - protein, 283 aa, Unknown Organism
  Description of Unknown Organism EGFR tyrosine kinase sequence
ILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMH

SEQ ID NO: 34 - protein, 6 aa, Artificial Sequence
  Description of Artificial Sequence 6-His tag
HHHHHH

SEQ ID NO: 35 - protein, 8 aa, Artificial Sequence
  Description of Artificial Sequence Synthetic N-terminal peptide
EVHHHHHH

SEQ ID NO: 36 - protein, 8 aa, Homo sapiens
DIYAEIPD

SEQ ID NO: 37 - protein, 4 aa, Artificial Sequence
  Description of Artificial Sequence Synthetic pET15S peptide fragment
GSHM

SEQ ID NO: 38 - protein, 7 aa, Homo sapiens
IYAEIPD